(19) United States
(12) Patent Application Publication
(10) Pub. No.: US 2025/0346597 A1
(43) Pub. Date: Nov. 13, 2025

(54) CONDENSED HETEROCYCLIC COMPOUNDS AS INHIBITOR OF DIACYLGLYCEROL KINASES

(71) Applicant: BEONE MEDICINES I GMBH, Basel (CH)

(72) Inventors: Zhikun Ni, Beijing (CN); Guoliang Zhang, Beijing (CN); Jianzhuang Miao, Beijing (CN); Zhi Zhang, Beijing (CN); Ce Wang, Beijing (CN)

(73) Assignee: BEONE MEDICINES I GMBH, Basel (CH)

(21) Appl. No.: 19/277,577

(22) Filed: Jul. 23, 2025

Related U.S. Application Data
(63) Continuation of application No. PCT/CN2024/075490, filed on Feb. 2, 2024.

(30) Foreign Application Priority Data
Feb. 2, 2023 (WO) PCT/CN2023/074240

(51) Int. Cl. C07D 471/04; A61K 31/496; A61K 31/498; A61K 31/502; A61K 31/5025; A61K 31/506; A61K 31/519; A61K 31/541; C07B 59/00; C07D 487/04; C07D 519/00

(52) U.S. Cl. CPC C07D 471/04; A61K 31/496; A61K 31/498; A61K 31/502; A61K 31/5025; A61K 31/506; A61K 31/519; A61K 31/541; C07B 59/002; C07D 487/04; C07D 519/00

(57) ABSTRACT

Disclosed herein is a compound of Formula (I), which is a DGKs inhibitor, for activating T cells, promoting T cell proliferation, and/or exhibiting antitumor activity, a method of using the compounds disclosed herein for treating cancer, and a pharmaceutical composition comprising the same.

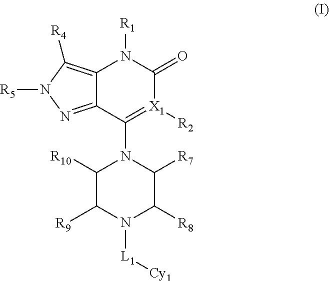

### CROSS-REFERENCE TO RELATED APPLICATIONS

This application is a continuation of International Application No. PCT/CN2024/075490, filed Feb. 2, 2024, entitled “Condensed Heterocyclic Compounds as Inhibitor of Diacylglycerol Kinases,” which claims the benefit of priority of PCT Application No. PCT/CN2023/074240, filed Feb. 2, 2023, entitled “Pyrazolopyridinone Compounds,” each of which is hereby incorporated by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on May 8, 2024, is named “01368-0104-00PCT.xml” and is 6,544 bytes in size. The sequence listing contained in this .XML file is part of the specification and is hereby incorporated by reference herein in its entirety.

### FIELD OF THE DISCLOSURE

Disclosed herein is a compound of Formula (I) for activating T cells, promoting T cell proliferation, and/or exhibiting antitumor activity, a method of using the compounds disclosed herein for treating cancer, and a pharmaceutical composition comprising the same.

### BACKGROUND OF THE DISCLOSURE

Diacylglycerol kinases (DGKs) are a family of lipid kinases that phosphorylates and converts diacylglycerol (DAG) into phosphatidic acid (PA). As the substrate of DGKs, DAG is generated from inositol phospholipids and other phospholipids at the plasma membrane by phospholipase C (PLC) hydrolysis in response to the activation of various cell-surface receptors, including G-protein coupled receptors (GPCR) and immunoreceptor tyrosine-based activation motif (ITAM)-bearing receptors (Rhee, Sue Goo. Annual review of biochemistry. 2001, 70.1: 281-312). DAG is one of the key intracellular second messengers that recruits and activates many downstream effectors including protein kinase C (PKC), protein kinase D (PKD) families, and Ras guanyl nucleotide releasing proteins (RasGRPs), which in turn activates NF-κB and extracellular regulated kinase (ERK) pathways (Merida, Isabel, et al. Biochemical Journal. 2008, 409.1: 1-18, Joshi, Rohan P., et al. International Journal of Molecular Sciences. 2013,14.4: 6649-6673). By consuming DAG, DGK controls and tunes the threshold and duration of DAG mediated signaling. Mammalian DGK family comprises 10 different members, in which DGKα, DGK ζ and DGKδ are the three major isoforms that abundantly expressed in lymphoid tissues (Joshi, Rohan P., et al. International Journal of Molecular Sciences. 2013,14.4: 6649-6673).

Cancer immunotherapy is a type of cancer treatment to manipulate and boost host immune system to recognize and attack cancer cells. A vast majority of studies have focused on targeting immune checkpoint inhibitors, such as CTLA-4 and PD-1/PD-L1, to reinvigorate exhausted CD8⁺ T cells within tumor sites. It was emerged that peripheral T cell tolerance, which under normal circumstances prevents detrimental autoimmune disease, can be hijacked by tumors to prevent anti-tumor immune response during carcinogenesis (Nüssing, Simone, et al. Frontiers in Immunology. 2020, 11: 2461). T cell anergy is a one of the most important mechanisms of T cell tolerance and has been reported to occur in tumor infiltrated T cells, which contributes to the immunosuppressive nature of tumor microenvironment (Abe, Brian T., and Fernando Macian. Oncoimmunology. 2013, 2.2: e22679). Anergy-associated transcription factor early growth response gene2 (Egr2) directly binds to Dgka and Dgkz promoter and increases their expression (Zheng, Yan, et al. Journal of Experimental Medicine 2012, 209.12: 2157-2163; Zheng, Yan, et al. Molecular Immunology. 2013, 55.3-4: 283-291). In anergic T cells, both DGKα and DGKζ play critical roles to negatively regulate DAG-signaling downstream of TCR and reduce the strength of TCR activation (Chen, Shelley S., et al. Frontiers in Cell and Developmental Biology. 2016, 4: 130). Thus, immune cell expressed DGKα and DGKζ were investigated as potential targets to reverse the hyporesponsiveness of the tumor infiltrated T cells. It was demonstrated that genetic deletion of DGKα or DGKζ enhanced cytokine production and proliferation of T cells (Olenchock, Benjamin A., et al. Nature immunology. 2006, 7.11: 1174-1181; Zhong, Xiao-Ping, et al. Nature immunology. 2003, 4.9: 882-890). DGKα or DGKζ single knockout in both mouse or human chimeric antigen receptor (CAR)-T cells showed superior effector function as determined by enhanced in vitro cytotoxicity and cytokine secretion when cocultured with antigen expressing titled cells (Riese, Matthew J., et al. Cancer Research. 2013, 73.12: 3566-3577; Jung, In-Young, et al. Cancer Research. 2018, 78.16: 4692-4703). MesoCAR-transduced DGKα or DGK deficient T cells also showed significantly elevated in vivo activity against mesotheliomas (Riese, Matthew J., et al. Cancer Research. 2013, 73.12: 3566-3577). DGKζ^{-/-} mice showed enhanced tumor suppressive efficacy with both orthotopic and subcutaneously implanted models (Wesley, Erin M., et al. Immunohorizons. 2018, 2,4: 107-118; Wee, Susan, et al. AACR; Cancer Res 2019; 79 (13 Suppl):Abstract nr 936). Besides the T cell regulatory function, both DGKα and DGKζ also involve in tuning NK cell activation at tumor site (Prinz, Petra U., et al. International Journal of Cancer. 2014. 135.8: 1832-1841; Yang, Enjun, et al. The Journal of Immunology. 2016, 197.3: 934-941). In addition, DGKζ were found to play a critical role to control the activation threshold of mature B cells (Wheeler, Matthew L., et al. Science Signaling. 2013, 6.297: ra91-ra91). In summary, all these preclinical data suggest titled inhibition of DGKα and DGKζ could be therapeutic beneficial to promote immunity against cancer.

Although the existing anti-CTLA-4 and anti-PD-1 therapies have shown clear clinical benefits in a subset of patients with various tumor types, there are still unmet medical needs to develop novel immunotherapies to achieve robust and durable clinical anti-tumor efficacy. Pre-clinical data strongly suggests there is great potential of developing DGKα and DGKζ targeted therapies to improve antitumor immunity.

### SUMMARY OF THE DISCLOSURE

The above needs have been met by providing the compounds disclosed herein which have a novel core structure and show the desired inhibition of DGKα and DGKζ. In some embodiments, the compounds disclosed herein show the selective inhibitory activity of DGKα over DGKζ. In some embodiments, the compounds disclosed herein show the selective inhibitory activity of DGKζ over DGKα. In some embodiments, the compounds disclosed herein show the dual inhibitory activity of both DGKα and DGKζ. In some embodiments, the compounds disclosed herein show the closely inhibitory activity on DGKα and DGKζ.

Disclosed herein provides a compound of formula (I),

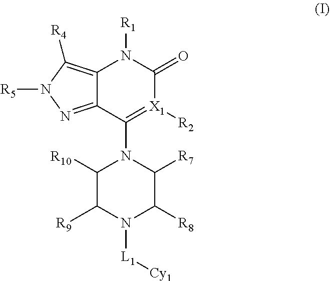

or a stereoisomer or pharmaceutically acceptable salt thereof
wherein
X₁ is C or N,
R₁ is hydrogen, or alkyl optionally substituted with deuterium, halogen, hydroxy, alkoxy or cycloalkyl;
R₂ is hydrogen, halogen, alkyl or cyano, provided that R₂ is absent when X₁ is N;
R₄ is hydrogen, halogen, or alkyl, wherein the alkyl is optionally substituted with deuterium or halogen;
R₅ is hydrogen, alkyl, alkenyl, alkynyl, cycloalkyl, cyano, or heterocyclyl, wherein said alkyl or alkenyl is unsubstituted or substituted with halogen, cyano, heterocyclyl, alkoxy, hydroxy, cycloalkyl;
each of R₇, R₉, R₈, and R₁₀ is independently hydrogen, alkyl, alkoxy, wherein said alkyl is unsubstituted or substituted with halogen, provided that at least one of R₇ and R₉ is not hydrogen;
L₁ is a direct bond, or —C(R_{L1})(R_{L2}), wherein said each of R_{L1}, R_{L2} is independently hydrogen or C₁₋₄alkyl optionally substituted with deuterium, halogen, alkyl, alkylene, alkynyl, cyano;
Cy₁ is aryl, heterocyclyl, heteroaryl, or cycloalkyl, each of which is unsubstituted or substituted with one, two or three substituents R₃ₐ, wherein each Ria is independently selected from hydroxy, alkoxy, alkyl, halogen, aminoalkyl, R_{3b}-SO2-, cycloalkyl, cyano, R_{3b}—C(O)—N(R_{3c})—, N(R_{3b}R_{3c})—C(O)—, N(R_{3b}R_{3c})—, R_{3b}—O—C(O)—, cycloalkyl, heterocyclyl or heterocyclyloxy, each alkyl moiety of which is unsubstituted or substituted with deuterium, halogen, alkoxy, hydroxy, cyano or heterocyclyl; wherein said cycloalkyl or heterocyclyl is unsubstituted or substituted with alkoxy, alkyl, halogen, or hydroxy, wherein R_{3b} and R_{3c} are each independently hydrogen or alkyl, or R_{3b} and R_{3c} together form a bridge containing at least one —CH₂— moiety;
provided that the compound is not 7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 7-((2S,5R)-2,5-dimethyl-4-(1-(quinoxalin-6-yl)ethyl)piperazin-1-yl)-2,4-dimethyl-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-4-methyl-2-(2,2,2-trifluoroethyl)-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-2-(2-methoxyethyl)-4-methyl-2,4-dihydro-5H-pyrazolo[4,3-b]pyridin-5-one, 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)acetamide, methyl 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)acetate, 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)propanenitrile, 7-((2S,5R)-2,5-dimethyl-4-(1-(quinoxalin-6-yl)ethyl)piperazin-1-yl)-2-(2-hydroxyethyl)-4-methyl-2,4-dihydro-5H-pyrazolo[4,3-b]pyridin-5-one, 2-(cyclopropylmethyl)-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-2-(2-(dimethylamino)ethyl)-4-methyl-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-4-methyl-2-(oxiran-2-ylmethyl)-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-4-methyl-2-(methylsulfonyl)-2H-pyrazolo[4,3-b]pyridin-5 (4H)-one, 2-cyclobutyl-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 2-(cyclopropanecarbonyl)-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 1-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)cyclopropane-1-carbonitrile, 2-cyclopropyl-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-2-isopropyl-4-methyl-2,4-dihydro-5H-pyrazolo[4,3-b]pyridin-5-one, 2-(3-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)-1-(ethylsulfonyl)azetidin-3-yl)acetonitrile, 3-cyclopentyl-3-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)propanenitrile, 7-((2S,5R)-2,5-dimethyl-4-(1-(quinoxalin-6-yl)ethyl)piperazin-1-yl)-4-methyl-2-(prop-2-yn-1-yl)-2,4-dihydro-5H-pyrazolo[4,3-b]pyridin-5-one, 2-(but-2-yn-1-yl)-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 2-(but-2-yn-1-yl)-7-((2S,5R)-2,5-diethyl-4-(1-(quinoxalin-6-yl)ethyl)piperazin-1-yl)-4-methyl-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-4-methyl-2-(prop-1-en-2-yl)-2,4-dihydro-5H-pyrazolo[4,3-b]pyridin-5-one, 7-((2S,5R)-2,5-diethyl-4-(1-(quinoxalin-6-yl)ethyl)piperazin-1-yl)-4-methyl-2-(prop-1-en-2-yl)-2,4-dihydro-5H-pyrazolo[4,3-b]pyridin-5-one, 7-((2S,5R)-2,5-dimethyl-4-(4-(trifluoromethoxy)benzyl)piperazin-1-yl)-4-methyl-2-(prop-1-en-2-yl)-2,4-dihydro-5H-pyrazolo[4,3-b]pyridin-5-one, 7-((2S,5R)-2,5-diethyl-4-(4-(trifluoromethoxy)benzyl)piperazin-1-yl)-4-methyl-2-(prop-1-en-2-yl)-2,4-dihydro-5H-pyrazolo[4,3-b]pyridin-5-one, 2-(7-((2S,5R)-2,5-diethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(methoxymethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-3-(methoxymethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-(methoxymethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,6-difluoro-4-methoxyphenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(5-fluoropyridin-2-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-([1,2,4]triazolo[1,5-a]pyridin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,5-difluoro-4-methoxyphenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-cyclopropylphenyl)propyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-phenylethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-methoxyphenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(isoquinolin-7-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(quinolin-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-methylbenzo[d]oxazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-methylbenzo[d]oxazol-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(quinoxalin-6-ylmethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-methylbenzo[d]oxazol-4-yl)ethyl)piperazin-1-yl)-4-methyl-5-oxo-4,5-dihydro-2H-pyrazolo[4,3-b]pyridin-2-yl)acetonitrile, mixture of 2-(7-((2S,5R)-4-(1-(3,3-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile, and 2-(7-((2S,5R)-4-(1-(2,2-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-4-ethyl-5-oxo-4,5-dihydro-2H-pyrazolo[4,3-b]pyridin-2-yl)acetonitrile, 2-(7-((2S,5R)-2,5-diethyl-4-(1-(isoquinolin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-([1,2,4]triazolo[1,5-a]pyridin-7-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(naphthalen-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-4-(methyl-d3)-5-oxo-4,5-dihydro-2H-pyrazolo[4,3-b]pyridin-2-yl)acetonitrile, 2-(7-((2S,5R)-4-(1-(benzo[d]thiazol-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-((4-fluoro-2-methoxyphenyl)(4-fluorophenyl)methyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(quinoxalin-6-ylmethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinolin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinolin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(isoquinolin-3-yl)ethyl)piperazin-1-yl)-4-methyl-5-oxo-4,5-dihydro-2H-pyrazolo[4,3-b]pyridine-2-yl)acetonitrile, 2-(7-((2S,5R)-2,5-diethyl-4-(1-(1-methyl-1H-benzo[d]imidazol-2-yl)ethyl)piperazin-1-yl)-4-methyl-5-oxo-4,5-dihydro-2H-pyrazolo[4,3-b]pyridine-2-yl)acetonitrile, 2-(7-((2S,5R)-2,5-diethyl-4-(1-(quinolin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinolin-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(isoquinolin-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(1,8-naphthyridin-2-yl)ethyl)-2,5-diethylpiperazin-1-yl)-⁴-methyl-5-oxo-4,5-dihydro-2H-pyrazolo[4,3-b]pyridin-2-yl)acetonitrile, 2-(7-((2S,5R)-2,5-diethyl-4-(1-(1-methyl-1H-benzo[d]imidazol-6-yl)ethyl)piperazin-1-yl)-4-methyl-5-oxo-4,5-dihydro-2H-pyrazolo[4,3-b]pyridin-2-yl)acetonitrile, mixture of 2-(7-((2S,5R)-2,5-diethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-diethyl-4-(1-(2-methylquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,2-difluorobenzo[d][1,3]dioxol-4-yl)ethyl)-2,5-diethylpiperazin-¹-yl)-⁴-methyl-5-oxo-4,5-dihydro-2H-pyrazolo[4,3-b]pyridin-2-yl)acetonitrile, 2-(7-((2S,5R)-2,5-diethyl-4-(1-(4-fluorophenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyridine-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-fluoro-4-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(4-fluorobenzyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-methoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-cyclopropyl-2-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-fluoro-4-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-fluoropyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(2,4-difluorobenzyl)-2,5-diethylpiperazin-1-yl)-4-methyl-5-oxo-4,5-dihydro-2H-pyrazolo[4,3-b]pyridine-2-yl)acetonitrile, 2-(7-((2S,5R)-4-(1-(4-cyclopropyl-2-fluorophenyl)propyl)-2,5-diethylpiperazin-1-yl)-4-methyl-5-oxo-4,5-dihydro-2H-pyrazolo[4,3-b]pyridine-2-yl)acetonitrile, 2-(7-((2S,5R)-2,5-diethyl-4-(4-(trifluoromethoxy)benzyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methoxy-4-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(methoxymethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-(trifluoromethoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-isopropoxypyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-3-methoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,6-difluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-methylphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(2-methoxyethoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyridin-4-yl)ethyl)piperazin-¹-yl)-⁴-methyl-5-oxo-4,5-dihydro-2H-pyrazolo[4,3-b]pyridin-2-yl)acetonitrile, 2-(7-((2S,5R)-4-(1-(3,5-difluoropyridin-4-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyrazin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-phenylethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methoxypyridin-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methoxypyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-(methoxymethyl)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2,6-dimethoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-4-(2-hydroxyethyl)-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-4-(2-methoxyethyl)-5-oxo-4,5-dihydro-2H-pyrazolo[4,3-b]pyridin-2-yl)acetonitrile, 2-(7-((2S,5R)-5-ethyl-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2-ethyl-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5S)-5-(hydroxymethyl)-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5S)-5-(methoxymethyl)-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2R,5R)-2-(hydroxymethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(quinoxalin-6-yl)ethyl)piperazin-1-yl)-5-oxo-4,5-dihydro-2H-pyrazolo[4,3-b]pyridin-2-yl)acetonitrile, 2-(7-((2S,5R)-2,5-diethyl-4-(1-(4-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-methyl-2-oxo-1,2-dihydropyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methoxypyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-(methoxymethyl)phenyl)ethyl)piperazin-1-yl)-⁴-methyl-5-oxo-4,5-dihydro-2H-pyrazolo[4,3-b]pyridin-2-yl)acetonitrile, 3-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)benzamide, N-(3-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)phenyl)acetamide, 2-(7-((2S,5R)-2,5-diethyl-4-(1-(3-methyl-2-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-bromo-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(methylsulfonyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-fluoro-4-(trifluoromethyl)pyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-methyl-2-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-methyl-2-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-chloro-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methyl-6-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-(trifluoromethyl)pyridin-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-5-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile, 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)benzamide, 2-(7-((2S,5R)-2,5-diethyl-4-(1-(3-(hydroxymethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(trifluoromethoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-5-oxo-4-(2,2,2-trifluoroethyl)-4,5-dihydro-2H-pyrazolo[4,3-b]pyridin-2-yl)acetonitrile, 2-(4-methyl-5-oxo-7-(5-(1-(quinoxalin-6-yl)ethyl)-2,5-diazabicyclo[2.2.2]octan-2-yl)-4,5-dihydro-2H-pyrazolo[4,3-b]pyridin-2-yl)acetonitrile, 2-(7-((2S,5R)-4-(1-(2,3-dihydrobenzo[b][1,4]dioxin-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(p-tolyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-(difluoromethyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-((S)-1-(quinoxalin-6-yl)ethyl)piperazin-1-yl)-5-oxo-4,5-dihydro-2H-pyrazolo[4,3-b]pyridin-2-yl)acetonitrile, 2-(7-((2S,5R)-2,5-diethyl-4-(1-(pyrimidin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyrimidin-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,4-difluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(6-chloro-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile, 2-(cyanomethyl)-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridine-6-carbonitrile, 2-(7-((2S,5R)-2,5-diethyl-4-(1-(6-(trifluoromethyl)pyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-fluoro-3-(trifluoromethyl)pyridin-2-yl)ethyl)piperazin-1-yl) 4-methyl-5-oxo-4,5-dihydro-2H-pyrazolo[4,3-b]pyridin-2-yl)acetonitrile, 2-(7-((2S,5R)-5-ethyl-4-(1-(4-fluoro-2-methoxyphenyl)ethyl)-2-methylpiperazin-1-yl)-4-methyl-5-oxo-4,5-dihydro-2H-pyrazolo[4,3-b]pyridin-2-yl)acetonitrile, 2-(7-((2S,5R)-4-(1-(3,4-dihydro-2H-benzo[b][1,4]oxazin-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-4-(2,2-difluoroethyl)-5-oxo-4,5-dihydro-2H-pyrazolo[4,3-b]pyridin-2-yl)acetonitrile, 2-(7-((2S,5R)-2,5-diethyl-4-(1-(5-isopropoxypyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-ethoxy-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-isopropoxyphenyl)ethyl)piperazin-1-yl)-4-methyl-5-oxo-4,5-dihydro-2H-pyrazolo[4,3-b]pyridin-2-yl)acetonitrile, 2-(7-((2R,5R)-2-(methoxymethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2-(2-hydroxyethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrilecetonitrile, 2-(7-((2S,5R)-2,5-diethyl-4-(1,2,3,4-tetrahydronaphthalen-1-yl)piperazin-1-yl)-4-methyl-5-oxo-4,5-dihydro-2H-pyrazolo[4,3-b]pyridin-2-yl)acetonitrile, 2-(7-((2S,5R)-4-(1-(3,5-difluoropyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(2,3-dihydro-1H-inden-1-yl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(chroman-4-yl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-((dimethylamino)methyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(hydroxymethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-methoxyphenyl)ethyl)piperazin-1-yl)-4-(2-hydroxyethyl)-5-oxo-4,5-dihydro-2H-pyrazolo[4,3-b]pyridin-2-yl)acetonitrile, 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(quinoxalin-2-yl)ethyl)piperazin-1-yl)-4-methyl-5-oxo-4,5-dihydro-2H-pyrazolo[4,3-b]pyridin-2-yl)acetonitrile, 2-(cyanomethyl)-7-((2S,5R)-5-ethyl-2-methyl-4-(1-(quinoxalin-6-yl)ethyl)piperazin-1-yl)-4-methyl-5-oxo-4,5-dihydro-2H-pyrazolo[4,3-b]pyridine-6-carbonitrile, 2-(7-((2S,5R)-2,5-diethyl-4-(1-(2-methoxy-4-(trifluoromethyl)phenyl)ethyl)piperazin-1-yl)-4-methyl-5-oxo-4,5-dihydro-2H-pyrazolo[4,3-b]pyridin-2-yl)acetonitrile, 2-((2S,5R)-1-(2-(cyanomethyl)-4-methyl-5-oxo-4,5-dihydro-2H-pyrazolo[4,3-b]pyridin-7-yl)-5-methyl-4-(1-(quinoxalin-6-yl)ethyl)piperazin-2-yl)acetonitrile, 2-(7-((2S,5R)-4-(1-(2,3-dihydro-[1,4]dioxino[2,3-b]pyridin-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-isopropoxyphenyl)ethyl)piperazin-1-yl)-4-methyl-5-oxo-4,5-dihydro-2H-pyrazolo[4,3-b]pyridin-2-yl)acetonitrile, 2-(7-((3R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(6-chloro-7-((3R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(methylamino)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2-(2-methoxyethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, N-(2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)phenyl)acetamide, 2-(7-((2S,5R)-2,5-diethyl-4-(1-(1-methyl-2,3-dihydro-1H-pyrido[2,3-b][1,4]oxazin-6-yl)ethyl)piperazin-1-yl)-4-methyl-5-oxo-4,5-dihydro-2H-pyrazolo[4,3-b]pyridin-2-yl)acetonitrile, 2-(4-(cyclopropylmethyl)-7-((2S,5R)-2,5-diethyl-4-(1-(quinoxalin-6-yl)ethyl)piperazin-1-yl)-5-oxo-4,5-dihydro-2H-pyrazolo[4,3-b]pyridin-2-yl)acetonitrile, 2-(7-((2S,5R)-2,5-dimethyl-4-(1-(quinoxalin-6-yl)ethyl)piperazin-1-yl)-6-fluoro-4-methyl-5-oxo-4,5-dihydro-2H-pyrazolo[4,3-b]pyridin-2-yl)acetonitrile, 2-(cyanomethyl)-7-((2S,5R)-3-ethyl-4-(1-(quinoxalin-6-yl)ethyl)piperazin-1-yl)-4-methyl-5-oxo-4,5-dihydro-2H-pyrazolo[4,3-b]pyridine-6-carbonitrile, 2-(7-((2S,5R)-2,5-diethyl-4-(1-(4-fluoro-2-(trifluoromethoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-(difluoromethoxy)-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(difluoromethyl)-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, methyl 4-(2-(cyanomethyl)-4-methyl-5-oxo-4,5-dihydro-2H-pyrazolo[4,3-b]pyridin-7-yl)-1-(1-(quinoxalin-6-yl)ethyl)piperazine-2-carboxylate, 2-(7-((2S,5R)-2,5-diethyl-4-(1-(1-ethyl-1H-imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-cyclopropyl-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-1H-indol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(quinoxalin-6-yl)ethyl)piperazin-1-yl)-5-oxo-4,5-dihydro-2H-pyrazolo[4,3-b]pyridin-2-yl)acetonitrile, 2-(4-methyl-7-((3S)-3-methyl-4-(1-(quinoxalin-6-yl)ethyl)piperazin-1-yl)-5-oxo-4,5-dihydro-2H-pyrazolo[4,3-b]pyridin-2-yl)acetonitrile, 2-(6-bromo-7-((2S,5R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-(difluoromethoxy)pyridin-3-yl)ethyl)-2,5-diethylpiperazin-1-yl)-4-methyl-5-oxo-4,5-dihydro-2H-pyrazolo[4,3-b]pyridin-2-yl)acetonitrile, mixture of 2-(7-((2S,5R)-2,5-diethyl-4-(1-(1-ethyl-5-methyl-1H-imidazol-2-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-diethyl-4-(1-(1-ethyl-4-methyl-1H-imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-4,5,6,7-tetrahydro-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(quinoxalin-6-yl)ethyl)piperazin-1-yl)-5-oxo-4,5-dihydro-2H-pyrazolo[4,3-d]pyrimidin-2-yl)acetonitrile, 2-(7-(3-(hydroxymethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-ethyl-3H-imidazo[4,5-b]pyridin-2-yl)ethyl)piperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-2,5-diethyl-4-(1-(1-ethyl-1H-benzo[d]imidazol-2-yl)ethyl)piperazin-1-yl)-4-methyl-2,4-dihydro-5H-pyrazolo[4,3-b]pyridin-5-one, 2-(7-((2S,5R)-4-(1-(5-chloro-1-ethyl-1H-imidazol-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(1-hydroxyethyl)phenyl)ethyl)piperazin-1-yl)-4-methyl-5-oxo-4,5-dihydro-2H-pyrazolo[4,3-b]pyridin-2-yl)acetonitrile, 2,2′-(6-cyano-7-((2S,5R)-5-ethyl-2-methyl-4-(1-(quinoxalin-6-yl)ethyl)piperazin-1-yl)-5-oxo-2H-pyrazolo[4,3-b]pyridine-2,4 (5H)-diyl)diacetonitrile, 2-(7-((2S,5R)-2,5-diethyl-4-(1-(quinoxalin-6-yl)ethyl)piperazin-1-yl)-4-isopropyl-5-oxo-4,5-dihydro-2H-pyrazolo[4,3-b]pyridin-2-yl)acetonitrile, 2-(7-((2S,5R)-4-(1-(4-chloro-1-ethyl-1 H-imidazol-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-propyl-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-isopropyl-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(quinoxalin-6-yl)ethyl)piperazin-1-yl)-6-fluoro-5-oxo-4,5-dihydro-2H-pyrazolo[4,3-b]pyridin-2-yl)acetonitrile, 2-(7-((2S,5R)-4-(1-(2-fluoro-4-(trifluoromethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-(trifluoromethyl)phenyl)ethyl)piperazin-1-yl)-4-methyl-5-oxo-4,5-dihydro-2H-pyrazolo[4,3-b]pyridin-2-yl)acetonitrile, 4-(2-(cyanomethyl)-4-methyl-5-oxo-4,5-dihydro-2H-pyrazolo[4,3-b]pyridin-7-yl)-1-(1-(quinoxalin-6-yl)ethyl)piperazine-2-carboxamide, 2-(7-((2S,5R)-4-(1-(4-cyclopropyl-2-methoxyphenyl)ethyl)-2,5-diethylpiperazin-1-yl)-4-methyl-5-oxo-4,5-dihydro-2H-pyrazolo[4,3-b]pyridin-2-yl)acetonitrile, 2-(cyanomethyl)-7-((2S,5R)-5-ethyl-2-methyl-4-(1-(quinoxalin-6-yl)ethyl)piperazin-1-yl)-5-oxo-4,5-dihydro-2H-pyrazolo[4,3-b]pyridine-6-carbonitrile, 2-(7-((2S,5R)-4-(1-(2,4-difluoro-6-methoxyphenyl)ethyl)-2,5-diethylpiperazin-1-yl)-⁴-methyl-5-oxo-4,5-dihydro-2H-pyrazolo[4,3-b]pyridin-2-yl)acetonitrile, 2-(7-((2S,5R)-5-ethyl-4-(1-(2-fluoro-4-(trifluoromethyl)phenyl)ethyl)-2-methylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-5-(trifluoromethyl)-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-6-(trifluoromethyl)-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-ethyl-4-fluorophenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(3-(trifluoromethyl)benzoyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-6-oxo-1,6-dihydropyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-2-methyl-1H-imidazol-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(quinoxalin-6-yl)ethyl)piperazin-1-yl)-3,4-dimethyl-5-oxo-4,5-dihydro-2H-pyrazolo[4,3-b]pyridin-2-yl)acetonitrile, 2-(7-((2S,5R)-2,5-diethyl-4-(1-(1-ethyl-3-(trifluoromethyl)-1H-pyrazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-3-(trifluoromethyl)-1H-pyrazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(3-chloro-1-ethyl-1H-pyrazol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-ethyl-1-methyl-1H-pyrazol-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-(2-hydroxyethyl)-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-1H-imidazo[4,5-b]pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-4-methyl-1H-pyrazol-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-(trifluoromethyl)pyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-4-(1-(4-fluoro-2-(trifluoromethyl)phenyl)ethyl)-2-methylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-2-methyl-1H-imidazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-d]pyrimidin-2-yl)acetonitrile, or 2-(7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-d]pyrimidin-2-yl)acetonitrile, 2-ethyl-7-((2S,5R)-5-ethyl-2-methyl-4-(1-(quinoxalin-6-yl)ethyl)piperazin-1-yl)-4-methyl-2,4-dihydro-5H-pyrazolo[4,3-b]pyridin-5-one, 2-(2,2-difluoroethyl)-7-((2S,5R)-5-ethyl-2-methyl-4-(1-(quinoxalin-6-yl)ethyl)piperazin-1-yl)-4-methyl-2,4-dihydro-5H-pyrazolo[4,3-b]pyridin-5-one, 2-(7-((2S,5R)-4-(1-cyclohexylethyl)-2,5-dimethylpiperazin-1-yl)-4-methyl-5-oxo-4,5-dihydro-2H-pyrazolo[4,3-b]pyridin-2-yl)acetonitrile, 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluorobenzonitrile, 2-(7-((2R,5R)-5-ethyl-2-(methoxymethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-chloro-4-(trifluoromethyl)pyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-4-methyl-1H-pyrazol-5-yl)ethyl)piperazin-1-yl)-4-methyl-5-oxo-4,5-dihydro-2H-pyrazolo[4,3-b]pyridin-2-yl)acetonitrile, 7-((2S,5R)-4-(1-(1-(2,2-difluoroethyl)-1H-pyrazol-5-yl)ethyl)-2,5-diethylpiperazin-1-yl)-4-methyl-2,4-dihydro-5H-pyrazolo[4,3-b]pyridin-5-one, 2-(7-((2R,5R)-5-ethyl-2-(hydroxymethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-7-(trifluoromethyl)-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 7-((2S,5R)-2,5-diethyl-4-(1-(2-fluoro-4-(trifluoromethyl)phenyl)ethyl)piperazin-1-yl)-4-methyl-2,4-dihydro-5H-pyrazolo[4,3-b]pyridin-5-one, 2-(7-((2S,5R)-2,5-diethyl-4-(1-(1-methyl-3-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-methyl-5-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5-(difluoromethyl)-1-methyl-1H-pyrazol-4-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-methyl-4-(trifluoromethyl)pyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(trifluoromethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(1-(2,2-difluoroethyl)-1H-imidazol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-2,5-diethyl-4-(1-(4-fluoro-2-(trifluoromethyl)phenyl)ethyl)piperazin-1-yl)-4-methyl-2,4-dihydro-5H-pyrazolo[4,3-b]pyridin-5-one, 2-(7-((2S,5R)-2,5-diethyl-4-(1-(1-ethyl-2-(trifluoromethyl)-1H-imidazol-5-yl)ethyl)piperazin-1-yl)-4-methyl-5-oxo-4,5-dihydro-2H-pyrazolo[4,3-b]pyridin-2-yl)acetonitrile, 3-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-1-ethyl-1H-pyrazole-4-carbonitrile, 2-(7-((2S,5R)-2,5-diethyl-4-(4-fluoro-2-(trifluoromethyl)benzyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(tetrahydro-2H-pyran-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(quinoxalin-6-yl)ethyl)piperazin-1-yl)-4-methyl-5-oxo-3-(trifluoromethyl)-4,5-dihydro-2H-pyrazolo[4,3-b]pyridin-2-yl)acetonitrile or 2-(7-((2S,5R)-2,5-dimethyl-4-((R)-1-(quinoxalin-6-yl)ethyl)piperazin-1-yl)-4-methyl-5-oxo-3-(trifluoromethyl)-4,5-dihydro-2H-pyrazolo[4,3-b]pyridin-2-yl)acetonitrile, 2-(7-((2S,5R)-2,5-dimethyl-4-((S)-1-(quinoxalin-6-yl)ethyl)piperazin-1-yl)-4-methyl-5-oxo-3-(trifluoromethyl)-4,5-dihydro-1H-pyrazolo[4,3-b]pyridin-1-yl)acetonitrile or 2-(7-((2S,5R)-2,5-dimethyl-4-((R)-1-(quinoxalin-6-yl)ethyl)piperazin-1-yl)-4-methyl-5-oxo-3-(trifluoromethyl)-4,5-dihydro-1H-pyrazolo[4,3-b]pyridin-1-yl)acetonitrile, 2-(3-chloro-7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-d]pyrimidin-2-yl)acetonitrile, or 2-(3-chloro-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-4-(4-fluoro-2-(trifluoromethyl)benzyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(difluoromethoxy)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-omethoxy-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(trifluoromethoxy)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,3-dihydro-[1,4]dioxino[2,3-b]pyridin-7-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-chloro-2-(trifluoromethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(difluoromethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(1,1-difluoroethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(quinoxalin-6-yl)propyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(trifluoromethyl)phenyl)propyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(trifluoromethyl)phenyl)ethyl)-2,5-dimethylpiperazin-1-yl)-4-(methyl-d3)-5-oxo-4,5-dihydro-2H-pyrazolo[4,3-b]pyridin-2-yl)acetonitrile, 2-(7-((2S,5R)-2,5-dimethyl-4-((R)-1-(quinoxalin-6-yl)ethyl)piperazin-1-yl)-3-fluoro-4-methyl-5-oxo-4,5-dihydro-1H-pyrazolo[4,3-b]pyridin-1-yl)acetonitrile, or 2-(7-((2S,5R)-2,5-dimethyl-4-((S)-1-(quinoxalin-6-yl)ethyl)piperazin-1-yl)-3-fluoro-4-methyl-5-oxo-4,5-dihydro-1H-pyrazolo[4,3-b]pyridin-1-yl)acetonitrile, 2-(7-((2R,5R)-5-ethyl-2-((R)-1-hydroxyethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((R)-1-(quinoxalin-6-yl)ethyl)piperazin-1-yl)-5-oxo-4,5-dihydro-2H-pyrazolo[4,3-b]pyridin-2-yl)acetonitrile, or 2-(7-((2S,5R)-2,5-dimethyl-4-((S)-1-(quinoxalin-6-yl)ethyl)piperazin-1-yl)-5-oxo-4,5-dihydro-2H-pyrazolo[4,3-b]pyridin-2-yl)acetonitrile, 2-(7-((2R,5R)-2-(difluoromethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(2,2-difluoro-1-(quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(3-chloro-7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-b]pyridin-2-yl)acetonitrile, or 2-(3-chloro-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(quinoxalin-6-yl)ethyl)piperazin-1-yl)-3-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(trifluoromethyl)phenyl)ethyl)piperazin-1-yl)-5-oxo-4,5-dihydro-2H-pyrazolo[4,3-b]pyridin-2-yl)acetonitrile, 2-(7-((2S,5R)-4-(1-(4-fluoro-2-(trifluoromethyl)phenyl)ethyl)-2,5-dimethylpiperazin-1-yl)-4-methyl-5-oxo-4,5-dihydro-2H-pyrazolo[4,3-d]pyrimidin-2-yl)acetonitrile, 2-(7-((2S,5R)-4-(1-(4-fluoro-2-(trifluoromethyl)phenyl)ethyl)-2,5-dimethylpiperazin-1-yl)-3,4-dimethyl-5-oxo-4,5-dihydro-2H-pyrazolo[4,3-d]pyrimidin-2-yl)acetonitrile, 2-(7-((2S,5R)-2,5-dimethyl-4-(1-(quinoxalin-6-yl-2,3-d2)ethyl)piperazin-1-yl)-4-methyl-5-oxo-4,5-dihydro-2H-pyrazolo[4,3-b]pyridin-2-yl)acetonitrile, mixture of 2-(7-((2S,5R)-2,5-dimethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-dimethyl-4-(1-(2-methylquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(2-methylquinoxalin-6-yl)ethyl)piperazin-1-yl)-4-methyl-5-oxo-4,5-dihydro-2H-pyrazolo[4,3-b]17yridine-2-yl)acetonitrile, 2-(7-((2S,5R)-2,5-dimethyl-4-((R)-1-(2-methylquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(3-methoxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(3-hydroxyoxetan-3-yl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,6-dichloro-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3,4-difluoro-2-methoxyphenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(quinoxalin-6-yl)ethyl)piperazin-1-yl)-6-methoxy-4-methyl-5-oxo-4,5-dihydro-2H-pyrazolo[4,3-b]pyridin-2-yl)acetonitrile, 7-((2S,5R)-2,5-diethyl-4-(1-(4-fluoro-2-(trifluoromethyl)phenyl)ethyl)piperazin-1-yl)-4-methyl-2,4-dihydro-5H-pyrazolo[4,3-b]pyridin-5-one, 2-(7-((2S,5R)-2,5-diethyl-4-(1-(4-fluoro-2-(trifluoromethyl)phenyl)ethyl)piperazin-1-yl)-4-(methyl-d3)-5-oxo-4,5-dihydro-2H-pyrazolo[4,3-b]pyridin-2-yl)acetonitrile, 2-(7-((2S,5R)-4-(1-(4-chloro-2-(trifluoromethyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-ethylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-hydroxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-chloroquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methoxyquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-hydroxyquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-methoxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-hydroxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(dimethylphosphoryl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(1-methoxyethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(cyanomethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3,4-difluoro-2-(trifluoromethyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluorobenzamide, methyl 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluorobenzoate, 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluoro-N-methylbenzamide, 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluoro-N,N-dimethylbenzamide, 2-(7-((2S,5R)-2,5-diethyl-4-(1-(4-fluoro-2-(trifluoromethyl)phenyl)propyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,3-dimethylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((3R,4R)-3-ethoxy-4-(3-(trifluoromethyl)phenoxy)piperidin-1-yl)-4-methyl-5-oxo-4,5-dihydro-2H-pyrazolo[4,3-b]pyridin-2-yl)acetonitrile, 2-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)-N-(2-methoxyethyl)-2-(4-(trifluoromethyl)phenyl)acetamide, 2-(7-((2S,5R)-4-(2-(4-fluorophenyl)propan-2-yl)-2,5-dimethylpiperazin-1-yl)-4-methyl-5-oxo-4,5-dihydro-2H-pyrazolo[4,3-b]pyridin-2-yl)acetonitrile, 2-(7-(2,5-dimethyl-4-(methyl(3-methylquinoxalin-6-yl)amino)piperidin-1-yl)-4-methyl-5-oxo-4,5-dihydro-2H-pyrazolo[4,3-b]pyridin-2-yl)acetonitrile, 2-allyl-7-((2S,5R)-2,5-dimethyl-4-((S)-1-(quinoxalin-6-yl)ethyl)piperazin-1-yl)-4-methyl-2,4-dihydro-5H-pyrazolo[4,3-b]pyridin-5-one, or 2-allyl-7-((2S,5R)-2,5-dimethyl-4-((R)-1-(quinoxalin-6-yl)ethyl)piperazin-1-yl)-4-methyl-2,4-dihydro-5H-pyrazolo[4,3-b]pyridin-5-one, 2-(but-3-en-1-yl)-7-((2S,5R)-2,5-dimethyl-4-((S)-1-(quinoxalin-6-yl)ethyl)piperazin-1-yl)-4-methyl-2,4-dihydro-5H-pyrazolo[4,3-b]pyridin-5-one, or 2-(but-3-en-1-yl)-7-((2S,5R)-2,5-dimethyl-4-((R)-1-(quinoxalin-6-yl)ethyl)piperazin-1-yl)-4-methyl-2,4-dihydro-5H-pyrazolo[4,3-b]pyridin-5-one, 2-allyl-7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one, 2-(but-2-en-1-yl)-7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one, 2-(3,3-difluoroallyl)-7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one, 2-(7-((2S,5R)-2,5-dimethyl-4-(1-(3-methylisoquinolin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(isoquinolin-3-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-methylquinolin-7-yl)ethyl)piperazin-1-yl)-4-methyl-5-oxo-4,5-dihydro-2H-pyrazolo[4,3-b]pyridin-2-yl)acetonitrile, 7-(1-((2R,5S)-4-(2-(cyanomethyl)-4-methyl-5-oxo-4,5-dihydro-2H-pyrazolo[4,3-b]pyridin-7-yl)-2,5-dimethylpiperazin-1-yl)ethyl)-2-naphthonitrile, 2-(7-((2S,5R)-2,5-dimethyl-4-((S)-1-(3-methylquinoxalin-6-yl)ethyl)piperazin-1-yl)-5-oxo-4,5-dihydro-2H-pyrazolo[4,3-b]pyridin-2-yl)acetonitrile, 2-(7-((2S,5R)-4-(1-(3-(hydroxymethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-cyclopropylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-(difluoromethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-(1,1-difluoroethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(3-(methyl-d3)quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(3-(trifluoromethyl)quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-(trifluoromethyl)quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-hydroxy-3-methylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(3-methylquinoxalin-6-yl)ethyl)piperazin-1-yl)-6-fluoro-4-methyl-5-oxo-4,5-dihydro-2H-pyrazolo[4,3-b]pyridin-2-yl)acetonitrile, 2-(cyanomethyl)-7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridine-6-carbonitrile, 2-(7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-dimethyl-4-(1-(2-methylbenzo[d]thiazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-methyl-[1,2,4]triazolo[1,5-a]pyridin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(benzo[d]thiazol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(3H-spiro[benzo[b][1,4]dioxine-2,1′-cyclopropan]-7-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,3-dihydro-[1,4]dioxino[2,3-b]pyridin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3,3-dimethyl-2,3-dihydro-[1,4]dioxino[2,3-b]pyridin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,2-dimethyl-2,3-dihydro-[1,4]dioxino[2,3-b]pyridin-7-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(1-methoxyethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-(1-(difluoromethoxy)ethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(1-methoxycyclopropyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(3-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4,4-difluorochroman-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-5-ethyl-4-((4-fluorophenyl)(5-(trifluoromethyl)pyridin-2-yl)methyl)-2-methylpiperazin-1-yl)-4-methyl-5-oxo-4,5-dihydro-2H-pyrazolo[4,3-b]pyridin-2-yl)acetonitrile, 2-(7-((2S,5R)-4-(di-p-tolylmethyl)-2,5-dimethylpiperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-2,5-diethyl-4-(1-(2-fluoro-4-(trifluoromethyl)phenyl)ethyl)piperazin-1-yl)-4-methyl-2,4-dihydro-5H-pyrazolo[4,3-b]pyridin-5-one, 2-(but-2-yn-1-yl)-7-((2S,5R)-4-(1-(3-(difluoromethyl)quinoxalin-6-yl)ethyl)-2,5-dimethylpiperazin-1-yl)-4-methyl-2,4-dihydro-5H-pyrazolo[4,3-b]pyridin-5-one, 2-(but-2-yn-1-yl)-7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one, 2-(7-((2S,5R)-4-(1-(2,3-dihydrofuro[2,3-b]pyridin-6-yl)ethyl)-2,5-diethylpiperazin-1-yl)-4-methyl-5-oxo-4,5-dihydro-2H-pyrazolo[4,3-b]pyridin-2-yl)acetonitrile, 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)phenyl)-2-methylpropanenitrile, 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)propyl)phenyl)-2-methylpropanenitrile, 2-(7-((2S,5R)-2,5-diethyl-4-(1-(2-fluoro-4-methylphenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-((2,2-dimethylmorpholino)methyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-((2,2-dimethylmorpholino)methyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-(((2S,6R)-2,6-dimethylmorpholino)methyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-((4,4-difluoropiperidin-1-yl)methyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-3-fluorophenyl)-2-methylpropanenitrile, 2-(7-((2S,5R)-2,5-diethyl-4-(1-(5-methylpyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5-(difluoromethyl)pyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-methylpyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-cyclopropylpyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(6-(difluoromethyl)pyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(3,3-dimethyl-2,3-dihydrofuro[3,2-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-fluoropyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoro-5-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5,6-dimethylpyrazin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-methylpyrazin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5,6,7,8-tetrahydroquinoxalin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-6-isopropoxypyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoro-5-methylpyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5-chloropyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-(trifluoromethoxy)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoro-5-(trifluoromethoxy)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5,6-dimethylpyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-methoxy-5-methylpyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-methylpyridazin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methylisoquinolin-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-methylisoquinolin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,6-naphthyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(4-cyclopropylbenzoyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(5-methylpicolinoyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(2,6-naphthyridine-3-carbonyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(isoquinoline-3-carbonyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(quinoline-7-carbonyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(quinoxaline-2-carbonyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-((tetrahydrofuran-3-yl)oxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-((tetrahydrofuran-3-yl)methoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-methoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(oxetan-3-yloxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(oxetan-3-ylmethoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-((tetrahydro-2H-pyran-4-yl)oxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-((4,4-difluoropiperidin-1-yl)methyl)pyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-(4,4-difluoropiperidine-1-carbonyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(morpholinomethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(3,4-dimethylphenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(4,5-dimethylpyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(6-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)pyridin-3-yl)-2-methylpropanenitrile, 2-(7-((2S,5R)-2,5-diethyl-4-(1-(3-fluoro-5-isopropoxypyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(imidazo[1,2-a]pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyrazolo[1,5-a]pyridin-2-yl)ethyl)piperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-4-(1-(2,2-dimethylbenzo[d][1,3]dioxol-5-yl)ethyl)-2,5-dimethylpiperazin-1-yl)-4-methyl-2,4-dihydro-5H-pyrazolo[4,3-b]pyridin-5-one, 2-(but-2-yn-1-yl)-7-((2S,5R)-4-(1-(2,2-difluorobenzo[d][1,3]dioxol-5-yl)ethyl)-2,5-dimethylpiperazin-1-yl)-4-methyl-2,4-dihydro-5H-pyrazolo[4,3-b]pyridin-5-one, 2-(but-2-yn-1-yl)-7-((2S,5R)-4-(1-(2,2-difluorobenzo[d][1,3]dioxol-5-yl)ethyl)-5-ethyl-2-methylpiperazin-1-yl)-4-methyl-2,4-dihydro-5H-pyrazolo[4,3-b]pyridin-5-one, 2-(but-2-yn-1-yl)-7-((2S,5R)-5-ethyl-4-(1-(isoquinolin-3-yl)ethyl)-2-methylpiperazin-1-yl)-4-methyl-2,4-dihydro-5H-pyrazolo[4,3-b]pyridin-5-one, 2-(but-2-yn-1-yl)-7-((2S,5R)-5-ethyl-2-methyl-4-(1-(quinoxalin-2-yl)ethyl)piperazin-1-yl)-4-methyl-2,4-dihydro-5H-pyrazolo[4,3-b]pyridin-5-one, or 2-(but-2-yn-1-yl)-7-((2S,5R)-5-ethyl-2-methyl-4-(1-(quinolin-7-yl)ethyl)piperazin-1-yl)-4-methyl-2,4-dihydro-5H-pyrazolo[4,3-b]pyridin-5-one.
wherein the variables are defined as herein.

### The Definitions of R₁

In some embodiments, R₁ is hydrogen, or alkyl optionally substituted with deuterium, halogen, hydroxy, alkoxy or cycloalkyl.

In some embodiments, R₁ is hydrogen, or C₁₋₄ alkyl optionally substituted with deuterium, halogen, hydroxy, alkoxy or cycloalkyl; In some embodiments, R₁ is hydrogen, or C₁₋₃ alkyl optionally substituted with deuterium or halogen.

In some embodiments, R₁ is hydrogen, methyl, ethyl, isopropyl, n-propyl, methyl-d3; In some embodiments, R₁ is hydrogen, methyl, ethyl, methyl-d3; more preferably R₁ is methyl.

### The Definitions of R₂ and X₁

In some embodiments, X₁ is N; In some embodiments, X₁ is C.

In some embodiments, R₂ is hydrogen, halogen, alkyl or cyano, provided that R₂ is absent when X₁ is N.

In some embodiments, R₂ is hydrogen, halogen, C₁₋₄alkyl or cyano, provided that X, is C; preferably R₂ is hydrogen, F, Br, C₁, methyl, ethyl or —CN; more preferably R₂ is F, Br, methyl or —CN.

### The Definitions of R₄

In some embodiments, R₄ is hydrogen, halogen, or alkyl, wherein the alkyl is optionally substituted with halogen or deuterium; In some embodiments, R₄ is hydrogen, deuterium, halogen or alkyl optionally substituted with halogen or deuterium; In some embodiments, R₄ is hydrogen, methyl or methyl-d3; In some embodiments, R₄ is hydrogen.

### The Definitions of R₅

In some embodiments, R₅ is hydrogen, alkyl, alkenyl, alkynyl, cycloalkyl, cyano, or heterocyclyl, wherein said alkyl or alkenyl is unsubstituted or substituted with halogen, cyano, heterocyclyl, alkoxy, hydroxy, cycloalkyl.

In some embodiments, R₅ is hydrogen, alkyl, alkenyl or alkynyl, wherein said alkyl is unsubstituted or substituted with cyano, cycloalkyl or cycloalkoxy; In some embodiments, R₅ is C₁₋₆alkyl, C₂₋₆alkenyl or C₂₋₆alkynyl, wherein said alkyl is substituted with cyano, C₃.cycloalkyl or C₂₋₆cycloalkoxy.

In some embodiments, R₅ is hydrogen, —CH₂—CN, —CH(CH₃)CN, —CH₂—CH₂—CN, —CH₂—CH₂—CH₂—CN, —CH(CH₃)—CH₂—CN, —CH₂—CH(CH₃)—CN, —CH(CH₂CH₃)—CN, oxiran-2-ylmethyl, oxiran-2-yl, oxetane-3-ylmethyl, oxetane-2-methyl, oxetane-3-yl, oxetane-2-yl, prop-2-yn-1-yl, but-2-yn-1-yl, but-3-yn-1-yl, pent-2-yn-1-yl, pent-3-yn-1-yl, pent-4-yn-1-yl, prop-2-en-1-yl, but-2-en-1-yl, but-3-en-1-yl, pent-2-en-1-yl, pent-3-en-1-yl, pent-4-en-1-yl, methyl, ethyl, propyl, isopropyl, cyclopropyl, cyclobutyl, azetidine-2-yl, azetidine-3-yl, azetidine-3-ylmethyl, azetidine-1-yl, azetidine-1-ylmethyl, aziridine-1-yl, aziridine-1-ylmethyl, aziridine-2-yl, aziridine-2-ylmethyl, 1-cyanocyclopropyl, 2-cyanocyclopropyl or 2-cyanocyclobutyl; In some embodiments, R₅ is hydrogen, —CH₂—CN, —CH(CH₃)CN, —CH₂—CH₂—CN, CH₂—CH₂—CH₂—CN, —CH(CH₃)—CH₂—CN, —CH₂—CH(CH₃)—CN, —CH(CH₂CH₃)—CN, oxetane-3-ylmethyl, oxetane-2-methyl, oxetane-3-yl, oxetane-2-yl, prop-2-yn-1-yl, but-2-yn-1-yl, but-3-yn-1-yl or pent-3-yn-1-yl; In some embodiments, R₅ is CN—CH₂—, —CH₂—CH₂—CN, but-2-yn-1-yl, but-2-yn-1-yl, or oxetane-3-ylmethyl, oxetane-3-yl.

### the Definitions of R₇/R₉R₈/R₁₀

In some embodiments, each of R₇, R₉, R₈, and R₁₀ is independently hydrogen, alkyl, alkoxy, wherein said alkyl is unsubstituted or substituted with halogen, provided that at least one of R₇ and R₉ is not hydrogen.

In some embodiments, each of R₇ and R₉ is independently hydrogen, alkyl, wherein said alkyl is unsubstituted or substituted with halogen; In some embodiments, each of R₇ and R₉ is independently C₁₋₄alkyl; In some embodiments, each of R₇ and R₉ is independently C₁₋₂alkyl. In some embodiments, R₇ and R₉ are each independently hydrogen, methyl, ethyl, isopropyl, n-propyl.

In some embodiments, R₇ is methyl, and R₉ is methyl; In some embodiments, R₇ is ethyl, and R₉ is ethyl; In some embodiments, R₇ is methyl, and R₉ is ethyl.

In some embodiments, R₈ and R₁₀ are each hydrogen.

In some embodiments, the 2-position carbon on the piperazine ring is S-configuration, and the 5-position carbon on the piperazine ring is R-configuration.

### The Definitions of L₁

In some embodiments, L₁ is a direct bond, or —C(R_{L1})(R_{L2}), wherein said each of R_{L1}, R_{L2} is independently hydrogen or C₁₄alkyl optionally substituted with deuterium, halogen, alkyl, alkylene, alkynyl, cyano.

In some embodiments, Lᵢ is a direct bond, —C(R_{L1})(R_{L2}), wherein said each of R_{L1}, Ru, is independently hydrogen or C₁₋₄alkyl optionally substituted with deuterium, halogen; In some embodiments, L₁ is a direct bond, —CH₂—, —CH(CH₃)—, —CH(CD₃)-, —CH(CH₂CH₃)—, —CH(C₃H₇)—, —CH(CHF₂)—, or —C(CH₃)₂—; In some embodiments, L₁ is —CH(CH₂CH₃)—, —CH(CH₃)—, or —CH(CD₃)⁺.

In some embodiments, the compound of the formula of (I) is the compound of the formula (II),

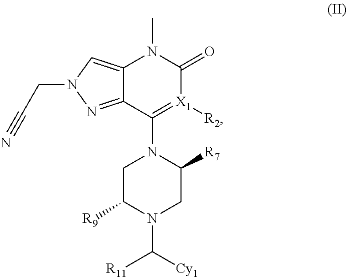

or a stereoisomer or a pharmaceutically acceptable salt thereof,
wherein Rₙ is methyl, methyl-d3, or ethyl, and the definitions of X₁, R₂, R₇, R₉ and Cy₁ are described as above.

In some embodiments, the carbon attached to R₁₁ and Cy₁ is R-configuration.

In some embodiments, the carbon attached to R₁₁ and Cy₁ is S-configuration.

In some embodiments, the compound of the formula of (II) is the compound of the formula (IIa),

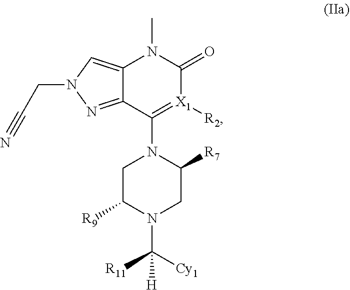

or a stereoisomer or a pharmaceutically acceptable salt thereof,
wherein each of R₁₁ is methyl or ethyl, and the definitions of X₁, R₂, R₇, R₉ and Cy₁ are described as above.

In some embodiments, the compound of the formula of (II) is the compound of the formula (IIb),

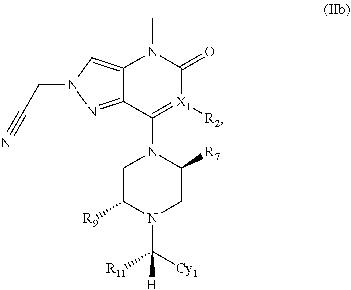

or a stereoisomer or a pharmaceutically acceptable salt thereof,
wherein each of R₁₁ is methyl or ethyl, and the definitions of X₁, R₂, R₇, R₉ and Cy₁ are described as above.

### The Definitions of Cy₁

In some embodiments, Cy₁ is aryl, heterocyclyl, heteroaryl, or cycloalkyl, each of which is unsubstituted or substituted with one, two or three substituents R₃ₐ, wherein each R₃ₐ is independently selected from hydroxy, alkoxy, alkyl, halogen, aminoalkyl, R_{3b}-SO2-, cycloalkyl, cyano, R_{3b}—C(O)—N(R_{3c})—, N(R_{3b}R_{3c})—C(O)—, N(R_{3b}R_{3c})—, R_{3b}—O—C(O)—, cycloalkyl, heterocyclyl or heterocyclyloxy, each alkyl moiety of which is unsubstituted or substituted with deuterium, halogen, alkoxy, hydroxy, cyano or heterocyclyl; wherein said cycloalkyl or heterocyclyl is unsubstituted or substituted with alkoxy, alkyl, halogen, or hydroxy, wherein R_{3b} and R_{3c} are each independently hydrogen or alkyl, or RA and R_{3c} together form a bridge containing at least one —CH₂— moiety

In some embodiments, R₃ₐ is selected from fluoro, bromo, chloro, methyl, ethyl, isopropyl, trifluoromethyl, hydroxyl, 1-methylpyrrolyl, 1-methylazetidinyl, 1-methylpiperidinyl, dimethylaminomethyl, 1,1-difluoroethyl, 3,3-difluoro-1-methylazetidinyl, dimethylaminyl, 1,1-dimethylmethyl, methoxy, cyclopropyl, cyclobutane, 2-fluoro-2-methylethyl, oxetan-3-ylmethyloxy, oxolane-3-ylmethyloxy, oxane-4-ylmethyloxy, oxetan-3-yloxy, oxolane-3-yloxy, oxane-3-yloxy, oxetan-3-methyl-3-yloxy, difluoromethoxy, 2-methoxyethoxy, (2-methoxyethoxy)methyl, 1,1-dioxidethiomorpholine-4-methyl, isopropoxy, dimethylcarbamoyl, (3-hydroxyazetidin-1-yl) methanone, formylamino, acetamido, propionamino, cyclopropoxy or amino, 4,4-difluoropiperidin-1-yl)methyl, oxetan-3-ylmethyl, (4-methoxypiperidin-1-yl)methyl, cyanomethyl, cyano, 1,1-dioxidothiomorpholino)methyl, methylcarbamoyl, 4-methoxypiperidine-1-carbonyl, (3-methoxypropylxmethyl)carbamoyl), (methylsulfonyl)methyl, 5-cyclopropoxy, 3-methoxypyrrolidine-1-carbonyl, 4-(3-methoxypyrrolidine-1-carbonyl)phenyl, 3-methoxypyrrolidine-1-carbonyl, or 4-(methoxymethyl.

In some embodiments, R₃ₐ is selected from fluoro, bromo, chloro, methyl, ethyl, isopropyl, trifluoromethyl, hydroxyl, 1-methylpyrrolyl, 1-methylazetidinyl, 1-methylpiperidinyl, dimethylaminomethyl, 1,1-difluoroethyl, 3,3-difluoro-1-methylazetidinyl, dimethylaminyl, 1,1-dimethylmethyl, methoxy, cyclopropyl, cyclobutane, 2-fluoro-2-methylethyl, oxetan-3-ylmethyloxy, oxolane-3-ylmethyloxy, oxane-4-ylmethyloxy, oxetan-3-yloxy, oxolane-3-yloxy, oxane-3-yloxy, oxetan-3-methyl-3-yloxy, difluoromethoxy, 2-methoxyethoxy, (2-methoxyethoxy)methyl, 1,1-dioxidethiomorpholine-4-methyl, isopropoxy, dimethylcarbamoyl, (3-hydroxyazetidin-1-yl) methanone, formylamino, acetamido, propionamino, cyclopropoxy or amino.

In some embodiments, Cy₁ is phenyl. In some embodiments, Cy₁ is phenyl, which is substituted with one Ria as disclosed herein at position 4 and optionally substituted with R₃ₐ on the other positions.

In some embodiments, Cy₁ is a monocyclic 5- to 9-membered heterocyclyl or a bicyclic 7- to 10-membered heterocyclyl which is unsubstituted or substituted with one, two or three R₃ₐ.

In some embodiments, monocyclic 5- to 9-membered heterocyclyl is

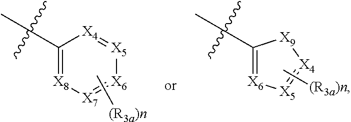

which is unsubstituted or substituted with one, two or three R₃ₐ, wherein each of X₄, X₅, X₆, X₇ and X₈ is independently selected from N or C, X₉ is C, N, S or O, provided that X₄, X₅, X₆, X₇ and X₈ are each independently unsubstituted when X₄, X₅, X₆, X₇ and X₈ are N.

In some embodiments, monocyclic 5- to 9-membered heterocyclyl is thiazole, isothiazole, triazole, pyridine, pyrazine, pyrimidine, each of which is unsubstituted or substituted with one, two or three R₃ₐ.

In some embodiments, monocyclic 5- to 9-membered heterocyclyl is thiazole, isothiazole, triazole, pyridine, pyrazine, pyrimidine, each of which is unsubstituted or substituted with one, two or three R₃ₐ.

In some embodiments, monocyclic 5- to 9-membered heterocyclyl is 4-(3-methoxyazetidine-1-carbonyl)phenyl, 4-(N,N-dimethylcarbamoyl)phenyl, 4-(N,N-dimethylcarbamoyl)-2-fluoro-phenyl, 4-((1,1-dioxidothiomorpholino)methyl)-3,5-difluorophenyl, 2,4-dimethylphenyl, 4-ethylphenyl, 3-methyl-4-(trifluoromethyl)phenyl, 4-methoxy-3-methylphenyl, 4-(1,1-difluoroethyl)phenyl, 4-(1,1-difluoroethyl)phenyl, 4-(2-methoxyethoxy)phenyl, 2-fluoro-4-methoxyphenyl, 2-fluoro-4-((tetrahydrofuran-3-yl)oxy)phenyl, 4-methoxy-2-methylphenyl, 4-(((tetrahydro-2H-pyran-4-yl)oxy)methyl)phenyl, 4-((oxetan-3-yloxy)methyl)phenyl, 4-(((tetrahydrofuran-3-yl)oxy)methyl)phenyl, 4-((2-methoxyethoxy)methyl)phenyl, 4-(difluoromethoxy)phenyl, 3-fluoro-4-methoxyphenyl, 3-fluoro-4-methoxyphenyl, 2-fluoro-4-(oxetan-3-yloxy)phenyl, 4-((3-methyloxetan-3-yl)oxy)phenyl, 4-((dimethylamino)methyl)-3,5-difluorophenyl, 4-(pyrrolidin-1-ylmethyl)phenyl, 4-((3,3-difluoroazetidin-1-yl)methyl)phenyl, 4-((dimethylamino)methyl)phenyl, 4-((methylamino)methyl)phenyl, 1-methyl-1H-1,2,3-triazol-4-yl)phenyl, 4-isopropylphenyl, 4-cyclopropoxyphenyl, 4-isopropoxyphenyl, 4-methoxyphenyl, 4,5-dimethylthiazol-2-yl, 6-cyclopropoxypyridin-3-yl, 6-cyclopropylpyridin-3-yl, 6-cyclopropyl-5-fluoropyridin-3-yl, 6-methoxypyridin-3-yl), 6-(methoxymethyl)pyridin-3-yl, 6-isopropylpyridin-3-yl, 6-ethylpyridin-3-yl, 6-cyclopropyl-2-fluoropyridin-3-yl, 2-fluoropropan-2-yl)pyridin-3-yl, 6-cyclopropyl-4-fluoropyridin-3-yl, 2-cyclopropylpyrimidin-5-yl, 6-methyl-5-(trifluoromethyl)pyridin-2-yl, 5-isopropoxypyridin-2-yl, 3,5-dimethylpyridin-2-yl, 5,6-dimethylpyridin-2-yl, 5-methoxy-6-methylpyridin-2-yl, 6-(methoxymethyl)-5-methylpyridin-2-yl, 5-cyclopropyl-3-methylpyridin-2-yl, 5-cyclopropyl-6-methylpyridin-2-yl, 5-methoxypyridin-2-yl, 5-methoxy-3-methylpyridin-2-yl, 1-(5-(methoxymethyl)pyridin-2-yl, 5-cyclopropyl-6-fluoropyridin-2-yl, 5-cyclopropyl-4-fluoropyridin-2-yl, ((4,4-difluoropiperidin-1-yl)methyl)pyridin-2-yl, 4-(oxetan-3-ylmethyl)phenyl, 6-aminopyridin-3-yl, 4-((4-methoxypiperidin-1-yl)methyl)phenyl, 4-(cyanomethyl)-phenyl, 4-cyano-phenyl, 4-((1,1-dioxidothiomorpholino)methyl)phenyl, 4-(methylcarbamoyl)phenyl, (4-methoxypiperidine-1-carbonyl)phenyl, 4-((3-methoxypropyl)(methyl)carbamoyl)phenyl, 4-((methylsulfonyl)methyl)phenyl, 6-(1,1-difluoroethyl)pyridin-3-yl, 5-cyclopropoxypyridin-2-yl, 4-(3-methoxypyrrolidine-1-carbonyl)phenyl, or 4-(methoxymethyl)phenyl.

In some embodiments, monocyclic 5- to 9-membered heterocyclyl is 4-(3-methoxyazetidine-1-carbonyl)phenyl, 4-(N,N-dimethylcarbamoyl)phenyl, 4-(N,N-dimethylcarbamoyl)-2-fluoro-phenyl, 4-((1,1-dioxidothiomorpholino)methyl)-3,5-difluorophenyl, 2,4-dimethylphenyl, 4-ethylphenyl, 3-methyl-4-(trifluoromethyl)phenyl, 4-methoxy-3-methylphenyl, 4-(1,1-difluoroethyl)phenyl, 4-(1,1-difluoroethyl)phenyl, 4-(2-methoxyethoxy)phenyl, 2-fluoro-4-methoxyphenyl, 2-fluoro-4-((tetrahydrofuran-3-yl)oxy)phenyl, 4-methoxy-2-methylphenyl, 4-(((tetrahydro-2H-pyran-4-yl)oxy)methyl)phenyl, 4-((oxetan-3-yloxy)methyl)phenyl, 4-(((tetrahydrofuran-3-yl)oxy)methyl)phenyl, 4-((2-methoxyethoxy)methyl)phenyl, 4-(difluoromethoxy)phenyl, 3-fluoro-4-methoxyphenyl, 3-fluoro-4-methoxyphenyl, 2-fluoro-4-(oxetan-3-yloxy)phenyl, 4-((3-methyloxetan-3-yl)oxy)phenyl, 4-((dimethylamino)methyl)-3,5-difluorophenyl, 4-(pyrrolidin-1-ylmethyl)phenyl, 4-((3,3-difluoroazetidin-1-yl)methyl)phenyl, 4-((dimethylamino)methyl)phenyl, 4-((methylamino)methyl)phenyl, 1-methyl-1H-1,2,3-triazol-4-yl)phenyl, 4-isopropylphenyl, 4-cyclopropoxyphenyl, 4-isopropoxyphenyl, 4-methoxyphenyl, 4,5-dimethylthiazol-2-yl, 6-cyclopropoxypyridin-3-yl, 6-cyclopropylpyridin-3-yl, 6-cyclopropyl-5-fluoropyridin-3-yl, 6-methoxypyridin-3-yl), 6-(methoxymethyl)pyridin-3-yl, 6-isopropylpyridin-3-yl, 6-ethylpyridin-3-yl, 6-cyclopropyl-2-fluoropyridin-3-yl, 2-fluoropropan-2-yl)pyridin-3-yl, 6-cyclopropyl-4-fluoropyridin-3-yl, 2-cyclopropylpyrimidin-5-yl, 6-methyl-5-(trifluoromethyl)pyridin-2-yl, 5-isopropoxypyridin-2-yl, 3,5-dimethylpyridin-2-yl, 5,6-dimethylpyridin-2-yl, 5-methoxy-6-methylpyridin-2-yl, 6-(methoxymethyl)-5-methylpyridin-2-yl, 5-cyclopropyl-3-methylpyridin-2-yl, 5-cyclopropyl-6-methylpyridin-2-yl, 5-methoxypyridin-2-yl, 5-methoxy-3-methylpyridin-2-yl, 1-(5-(methoxymethyl)pyridin-2-yl, 5-cyclopropyl-6-fluoropyridin-2-yl, or 5-cyclopropyl-4-fluoropyridin-2-yl.

In some embodiments, wherein Cy¹ is

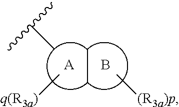

wherein each of ring A and ring B is, independently, a fused aryl, fused 5- to 6-membered heteroaryl, or fused 5- to 6-membered heterocyclyl;
and each of p and q is, independently, 0, 1, 2, 3, provided the sum of p and q is less than or equal to 3.

In some embodiments, ring A is fused 6-membered heterocyclyl, fused 6-membered aryl or fused 6-membered cycloalkyl; preferably pyridinyl, pyrazinyl, pyrimidinyl, pyridazinyl, or phenyl; more preferably pyridinyl pyrimidinyl, or pyridazinyl.

In some embodiments, ring B is fused 5-membered heterocyclyl, or fused 5-membered heteroaryl; preferably imidazol, thiazolyl, [1,2,4]triazolyl, tetrahydroimidazolyl, thiazolyl, pyrazolyl, thiazolyl, thienyl, oxazolyl, thiadiazolyl, thienyl, 1,2-dihydrothiazolyl, oxadiazolyl, [1,2,3]triazolyl, dioxolyl, or furanyl; more preferably thiazolyl, pyrazolyl, thiophenyl, or imidazol.

In some embodiments,

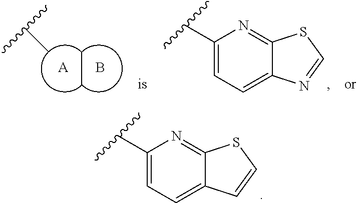

In some embodiments,

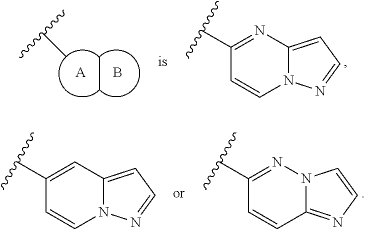

In some embodiments, bicyclic 7- to 10-membered heteroaryl is 6,7-dihydro-5H-cyclopenta[b]pyridine, 6,7-dihydro-5H-cyclopenta[c]pyridine, 2,3-dihydro-1H-indene, 2,3-dihydrobenzofuran, benzo[d][1,3]dioxole, 2,3-dihydrobenzo[b][1,4]dioxine, 4,5,6,7-tetrahydrobenzo[d]thiazole, 4,5,6,7-tetrahydrobenzo[d]isothiazole, quinoxalin, benzo[d]oxazole, benzo[d]thiazole, thiazolo[5,4-b]pyridine, thiazolo[4,5-b]pyridine, benzo[b]thiophene, benzofuran, benzo[c][1,2,5]thiadiazole, 1H-imidazo[4,5-b]pyridine, [1,2,4]triazolo[1,5-a]pyridine, pyrazolo[1,5-a]pyridine, imidazo[1,2-a]pyridine, pyrazolo[1,5-a]pyridine, imidazo[1,5-a]pyridine, benzo[c]isothiazole, thiazolo[4,5-c]pyridine, thiazolo[4,5-b]pyridine, thieno[2,3-b]pyridin, imidazo [1,2-b]pyridazine, pyrazolo[1,5-a]pyrimidine, thieno[3,2-b]pyridine, pyrazolo[1,5-a]pyridine, [1,2,4]triazolo[4,3-a]pyridine, phthalazine, isoquinoline, 2,3-dihydrofuro[2,3-c]pyridine, or 2,3-dihydrofuro[2,3-b]pyridine, each of which is unsubstituted or substituted with one, two or three R₃ₐ. In some embodiments, bicyclic 7- to 10-membered heteroaryl is 6,7-dihydro-5H-cyclopenta[b]pyridine, 6,7-dihydro-5H-cyclopenta[c]pyridine, 2,3-dihydro-1H-indene, 2,3-dihydrobenzofuran, benzo[d][1,3]dioxole, 2,3-dihydrobenzo[b][1,4]dioxine, 4,5,6,7-tetrahydrobenzo[d]thiazole, 4,5,6,7-tetrahydrobenzo[d]isothiazole, quinoxalin, benzo[d]oxazole, benzo[d]thiazole, thiazolo[5,4-b]pyridine, thiazolo[4,5-b]pyridine, benzo[b]thiophene, benzofuran, benzo[c][1,2,5]thiadiazole, 1H-imidazo[4,5-b]pyridine, [1,2,4]triazolo[1,5-a]pyridine, pyrazolo[1,5-a]pyridine, imidazo[1,2-a]pyridine, pyrazolo[1,5-a]pyridine, imidazo[1,5-a]pyridine, benzo[c]isothiazole, thiazolo[4,5-c]pyridine, thiazolo[4,5-b]pyridine, or thieno[2,3-b]pyridin, each of which is unsubstituted or substituted with one, two or three R₃ₐ.

In some embodiments, bicyclic 7- to 10-membered heteroaryl is 6,7-dihydro-5H-cyclopenta[b]pyridine-2-yl, 6,7-dihydro-5H-cyclopenta[b]pyridine-2-yl, 6,7-dihydro-5H-cyclopenta[c]pyridine-3-yl, 6,7-dihydro-5H-cyclopenta[c]pyridine-3-yl, 2,3-dihydro-1H-indene-5-yl, 2,3-dihydrobenzofuran-5-yl, 2,3-dihydrobenzofuran-6-yl, benzo[d][1,3]dioxole-5-yl, 2,3-dihydrobenzo[b][1,4]dioxine-6-yl, 4,5,6,7-tetrahydrobenzo[d]thiazole-2-yl, 4,5,6,7-tetrahydrobenzo[d]isothiazole-3-yl, quinolin-2-yl, quinolin-6-yl, benzo[d]oxazole-5-yl, benzo[d]oxazole-6-yl, benzo[d]thiazole-5-yl, benzo[d]thiazole-6-yl, thiazolo[5,4-b]pyridine-5-yl, thiazolo[5,4-b]pyridine-6-yl, thiazolo[4,5-b]pyridine-5-yl, thiazolo[4,5-b]pyridine-6-yl, benzo[b]thiophene-6-yl, benzofuran-6-yl, benzo[c][1,2,5]thiadiazole-5-yl, 1H-imidazo[4,5-b]pyridine-5-yl, 1H-imidazo[4,5-b]pyridine-6-yl, [1,2,4]triazolo[1,5-a]pyridine-5-yl, [1,2,4]triazolo[1,5-a]pyridine-6-yl, pyrazolo[1,5-a]pyridine, 5-yl, pyrazolo[1,5-a]pyridine-6-yl, imidazo[1,2-a]pyridine-5-yl, imidazo[1,2-a]pyridine-6-yl, pyrazolo[1,5-a]pyridine-5-yl, pyrazolo[1,5-a]pyridine-6-yl, imidazo[1,5-a]pyridine-5-yl, imidazo[1,5-a]pyridine-6-yl, benzo[c]isothiazole-5-yl, benzo[c]isothiazole-6-yl, thiazolo[4,5-c]pyridine-5-yl, thiazolo[4,5-c]pyridine-6-yl, thiazolo[4,5-b]pyridine-5-yl, thiazolo[4,5-b]pyridine-6-yl, thieno[2,3-b]pyridin-6-yl, thieno[2,3-b]pyridin-5-yl, 4-fluoro-2-methylbenzo[d]thiazol-5-yl, 2-(methoxymethyl)thiazolo[5,4-b]pyridin-5-yl, 2-(difluoromethyl)thieno[2,3-b]pyridine-6-yl, 2-(methoxymethyl)benzo[d]thiazol-6-yl, 2-cyclopropoxythiazolo[5,4-b]pyridin-5-yl, 2-(2-fluoroethoxy)thiazolo[5,4-b]pyridin-5-yl, 2-(difluoromethyl)thieno[2,3-b]pyridine-6-yl, 2-methylimidazo [1,2-b]pyridazin-6-yl, 3-methylpyrazolo[1,5-a]pyrimidin-5-yl, 2-methylpyrazolo[1,5-a]pyrimidin-5-yl, 2-ethylimidazo [1,2-b]pyridazin-6-yl, pyrazolo[1,5-a]pyrimidin-5-yl, 2-methylthieno[3,2-b]pyridin-5-yl, 2,3-dimethylpyrazolo[1,5-a]pyrimidin-5-yl, 2-fluoropyrazolo[1,5-a]pyrimidin-5-yl, pyrazolo[1,5-a]pyridin-5-yl, 3-fluoro-2-methylpyrazolo[1,5-a]pyrimidin-5-yl, 3-fluoropyrazolo[1,5-a]pyrimidin-5-yl, 2-methylpyrazolo[1,5-a]pyridin-5-yl, pyrazolo[1,5-a]pyrimidin-5-yl, 3-fluoropyrazolo[1,5-a]pyridin-5-yl, 3-chloroimidazo[1,2-b]pyridazin-6-yl, 3-fluoroimidazo[1,2-b]pyridazin-6-yl, 3-fluoro-2-methylpyrazolo[1,5-a]pyridin-5-yl, 2-chloropyrazolo[1,5-a]pyrimidin-5-yl, 2-chloro-3-fluoropyrazolo[1,5-a]pyrimidin-5-yl, 7-fluoro-3,3-dimethyl-2,3-dihydrobenzo[b][1,4]dioxin-6-yl, imidazo[1,2-b]pyridazin-2-yl, thiazolo[5,4-b]pyridin-2-yl, [1,2,4]triazolo[4,3-a]pyridin-7-yl, phthalazin-6-yl, phthalazin-5-yl, 1-methylisoquinolin-6-yl, 2,3-dihydrofuro[2,3-c]pyridin-5-yl, 2,2-difluorobenzo[d][1,3]dioxol-5-yl, 2,3-dihydrofuro[2,3-b]pyridin-6-yl, 4-((3-methoxypyrrolidin-1-yl)methyl)phenyl, 3-methylpyrazolo[1,5-a]pyridine-6-yl, or quinoxalin-2-yl, each of which is unsubstituted or substituted with one, two or three R₃ₐ. In some embodiments, bicyclic 7- to 10-membered heteroaryl is 6,7-dihydro-5H-cyclopenta[b]pyridine-2-yl, 6,7-dihydro-5H-cyclopenta[b]pyridine-2-yl, 6,7-dihydro-5H-cyclopenta[c]pyridine-3-yl, 6,7-dihydro-5H-cyclopenta[c]pyridine-3-yl, 2,3-dihydro-1H-indene-5-yl, 2,3-dihydrobenzofuran-5-yl, 2,3-dihydrobenzofuran-6-yl, benzo[d][1,3]dioxole-5-yl, 2,3-dihydrobenzo[b][1,4]dioxine-6-yl, 4,5,6,7-tetrahydrobenzo[d]thiazole-2-yl, 4,5,6,7-tetrahydrobenzo[d]isothiazole-3-yl, quinolin-2-yl, quinolin-6-yl, benzo[d]oxazole-5-yl, benzo[d]oxazole-6-yl, benzo[d]thiazole-5-yl, benzo[d]thiazole-6-yl, thiazolo[5,4-b]pyridine-5-yl, thiazolo[5,4-b]pyridine-6-yl, thiazolo[4,5-b]pyridine-5-yl, thiazolo[4,5-b]pyridine-6-yl, benzo[b]thiophene-6-yl, benzofuran-6-yl, benzo[c][1,2,5]thiadiazole-5-yl, 1H-imidazo[4,5-b]pyridine-5-yl, 1H-imidazo[4,5-b]pyridine-6-yl, [1,2,4]triazolo[1,5-a]pyridine-5-yl, [1,2,4]triazolo[1,5-a]pyridine-6-yl, pyrazolo[1,5-a]pyridine, 5-yl, pyrazolo[1,5-a]pyridine-6-yl, imidazo[1,2-a]pyridine-5-yl, imidazo[1,2-a]pyridine-6-yl, pyrazolo[1,5-a]pyridine-5-yl, pyrazolo[1,5-a]pyridine-6-yl, imidazo[1,5-a]pyridine-5-yl, imidazo[1,5-a]pyridine-6-yl, benzo[c]isothiazole-5-yl, benzo[c]isothiazole-6-yl, thiazolo[4,5-c]pyridine-5-yl, thiazolo[4,5-c]pyridine-6-yl, thiazolo[4,5-b]pyridine-5-yl, thiazolo[4,5-b]pyridine-6-yl, thieno[2,3-b]pyridin-6-yl, or thieno[2,3-b]pyridin-5-yl, each of which is unsubstituted or substituted with one, two or three R₃ₐ.

In some embodiments, bicyclic 7- to 10-membered heteroaryl is thiazolo[5,4-b]pyridine-5-yl, thieno[2,3-b]pyridin-6-yl, pyrazolo[1,5-a]pyrimidin-5-yl, pyrazolo[1,5-a]pyridine-5-yl, or imidazo[1,2-b]pyridazine-6-yl.

In some embodiments, Cy₁ is 4-(3-methoxyazetidine-1-carbonyl)phenyl, N,N-dimethylbenzamide-4-yl, 2,6-difluoro-N,N-dimethylbenzamide-4-yl, 2-fluoro-N,N-dimethylbenzamide-4-yl, 4-((1,1-dioxidothiomorpholino)methyl)-2,6-difluorophenyl, 2,4-dimethylphenyl, 4-ethylphenyl, 3-methyl-4-(trifluoromethyl)phenyl, 4-(1,1-difluoroethyl)phenyl, 4-(2-methoxyethoxy)phenyl, 2-fluoro-4-methoxyphenyl, 2-fluoro-4-((tetrahydrofuran-3-yl)oxy)phenyl, 4-methoxy-2-methylphenyl, 4-(((tetrahydro-2H-pyran-4-yl)oxy)methyl)phenyl, 4-((oxetan-3-yloxy)methyl)phenyl, 4-(((tetrahydrofuran-3-yl)oxy)methyl)phenyl, 4-((2-methoxyethoxy)methyl)phenyl, 4-(difluoromethoxy)phenyl, 3-fluoro-4-methoxyphenyl, 3-methyl-4-(oxetan-3-yloxy)phenyl, 2-fluoro-4-(oxetan-3-yloxy)phenyl, 3-fluoro-4-(oxetan-3-yloxy)phenyl, 4-((3-methyloxetan-3-yl)oxy)phenyl, 4-((dimethylamino)methyl)-3,5-difluorophenyl, 4-(pyrrolidin-1-ylmethyl)phenyl, 4-((3,3-difluoroazetidin-1-yl)methyl)phenyl, 4-((dimethylamino)methyl)phenyl, 4-((methylamino)methyl)phenyl, 4-(1-methyl-1H-1,2,3-triazol-4-yl)phenyl, 4-isopropylphenyl, 4-cyclopropoxyphenyl, 4-isopropoxyphenyl, 4-methoxyphenyl, 4,5-dimethylthiazol-2-yl, 4,5,6,7-tetrahydrobenzo[d]thiazol-2-yl, 6-cyclopropoxypyridin-3-yl, 6-cyclopropylpyridin-3-yl, 6-cyclopropyl-5-fluoropyridin-3-yl, 6-methoxypyridin-3-yl, 6-(methoxymethyl)pyridin-3-yl, 6-isopropylpyridin-3-yl, 6-ethylpyridin-3-yl, 6-cyclopropyl-2-fluoropyridin-3-yl, 6-(2-fluoropropan-2-yl)pyridin-3-yl, 6-cyclopropyl-4-fluoropyridin-3-yl, 2-cyclopropylpyrimidin-5-yl, 6-methyl-5-(trifluoromethyl)pyridin-2-yl, 5-isopropoxypyridin-2-yl, 5-cyclopropylpyridin-2-yl, 3,5-dimethylpyridin-2-yl, 5,6-dimethylpyridin-2-yl, 5-methoxy-6-methylpyridin-2-yl, 6-(methoxymethyl)-5-methylpyridin-2-yl, 5-cyclopropyl-3-methylpyridin-2-yl, 5-cyclopropyl-6-methylpyridin-2-yl, 5-methoxypyridin-2-yl, 5-methoxy-3-methylpyridin-2-yl, 1-(5-(methoxymethyl)pyridin-2-yl, 5-cyclopropyl-6-fluoropyridin-2-yl, 5-cyclopropyl-4-fluoropyridin-2-yl, 2,3-dihydro-1H-inden-5-yl, 1,1-dimethyl-2,3-dihydro-1H-inden-5-yl, 2,3-dihydrobenzofuran-5-yl, benzo[d][1,3]dioxol-5-yl, 4-fluoro-2,2-dimethylbenzo[d][1,3]dioxol-5-yl, 2,2-dimethylbenzo[d][1,3]dioxol-5-yl, 6,7-dihydro-5H-cyclopenta[b]pyridin-2-yl, 2,3-dihydrofuro[3,2-b]pyridin-5-yl, 2,3-dihydrofuro[2,3-b]pyridin-5-yl, [1,3]dioxolo[4,5-b]pyridin-5-yl, 3-fluoropyrazolo[1,5-a]pyridin-6-yl, pyrazolo[1,5-a]pyridin-6-yl, 3-methyl-3H-imidazo[4,5-b]pyridin-6-yl, 3-methylimidazo[1,2-a]pyridin-7-yl, imidazo[1,2-a]pyridin-7-yl, [1,2,4]triazolo[1,5-a]pyridin-6-yl, imidazo[1,2-a]pyridin-6-yl, imidazo[1,5-a]pyridin-6-yl, benzo[d]oxazol-6-yl, 7-fluoro-2-methylbenzo[d]oxazol-6-yl, 4-acetamido-2-fluoro-3-hydroxyphenyl, benzo[c]isothiazol-5-yl, benzo[b]thiophen-6-yl, benzo[d]thiazol-⁶-yl, 2-methylbenzo[d]thiazol-6-yl, 2-ethylbenzo[d]thiazol-6-yl, thiazolo[5,4-b]pyridin-5-yl, thiazolo[4,5-b]pyridin-6-yl, 4-fluorobenzo[d]thiazol-6-yl, 7-fluorobenzo[d]thiazol-6-yl, 5-fluorobenzo[d]thiazol-6-yl, 7-fluoro-2-methylbenzo[d]thiazol-6-yl, benzo[d]oxazol-5-yl, benzo[c][1,2,5]thiadiazol-5-yl, benzo[d]thiazol-5-yl, 2-methylbenzo[d]thiazol-5-yl, 2-ethylbenzo[d]thiazol-5-yl, 4-fluorobenzo[d]thiazol-5-yl, 3-methylquinoxalin-2-yl, 5-fluoro-2-methylbenzo[d]thiazol-6-yl, 2-methylthiazolo[5,4-b]pyridin-5-yl, 2-(difluoromethyl)thieno[2,3-b]pyridin-6-yl, thieno[2,3-b]pyridin-6-yl, 2-methylthieno[2,3-b]pyridin-6-yl, 2-fluorothieno[2,3-b]pyridin-6-yl, 6-(1,1-difluoroethyl)pyridin-3-yl, 4-trifluoromethyl-phenyl, 3-methylquinoxalin-6-yl, 3-(methyl-d3)quinoxalin-6-yl, 4-fluoro-2-methylbenzo[d]thiazol-5-yl, 2-(methoxymethyl)thiazolo[5,4-b]pyridin-5-yl, 2-(difluoromethyl)thieno[2,3-b]pyridine-6-yl, 2-(methoxymethyl)benzo[d]thiazol-6-yl, 2-cyclopropoxythiazolo[5,4-b]pyridin-5-yl, 2-(2-fluoroethoxy)thiazolo[5,4-b]pyridin-5-yl, 2-(difluoromethyl)thieno[2,3-b]pyridine-6-yl, 2-methylimidazo [1,2-b]pyridazin-6-yl, 3-methylpyrazolo[1,5-a]pyrimidin-5-yl, 2-methylpyrazolo[1,5-a]pyrimidin-5-yl, 2-ethylimidazo [1,2-b]pyridazin-6-yl, pyrazolo[1,5-a]pyrimidin-5-yl, 2-methylthieno[3,2-b]pyridin-5-yl, 2,3-dimethylpyrazolo[1,5-a]pyrimidin-5-yl, 2-fluoropyrazolo[1,5-a]pyrimidin-5-yl, pyrazolo[1,5-a]pyridin-5-yl, 3-fluoro-2-methylpyrazolo[1,5-a]pyrimidin-5-yl, 3-fluoropyrazolo[1,5-a]pyrimidin-5-yl, 2-methylpyrazolo[1,5-a]pyridin-5-yl, pyrazolo[1,5-a]pyrimidin-5-yl, 3-fluoropyrazolo[1,5-a]pyridin-5-yl, 3-chloroimidazo[1,2-b]pyridazin-6-yl, 3-fluoroimidazo[1,2-b]pyridazin-6-yl, 3-fluoro-2-methylpyrazolo[1,5-a]pyridin-5-yl, 2-chloropyrazolo[1,5-a]pyrimidin-5-yl, 2-chloro-3-fluoropyrazolo[1,5-a]pyrimidin-5-yl, 7-fluoro-3,3-dimethyl-2,3-dihydrobenzo[b][1,4]dioxin-6-yl, imidazo[1,2-b]pyridazin-2-yl, thiazolo[5,4-b]pyridin-2-yl, [1,2,4]triazolo[4,3-a]pyridin-7-yl, phthalazin-6-yl, phthalazin-5-yl, 1-methylisoquinolin-6-yl, 2,3-dihydrofuro[2,3-c]pyridin-5-yl, 2,2-difluorobenzo[d][1,3]dioxol-5-yl, 2,3-dihydrofuro[2,3-b]pyridin-6-yl, 4-((3-methoxypyrrolidin-1-yl)methyl)phenyl, 3-methylpyrazolo[1,5-a]pyridine-6-yl, quinoxalin-2-yl.

In some embodiments, Cy₁ is 4-(3-methoxyazetidine-1-carbonyl)phenyl, N,N-dimethylbenzamide-4-yl, 2,6-difluoro-N,N-dimethylbenzamide-4-yl, 2-fluoro-N,N-dimethylbenzamide-4-yl, 4-((1,1-dioxidothiomorpholino)methyl)-2,6-difluorophenyl, 2,4-dimethylphenyl, 4-ethylphenyl, 3-methyl-4-(trifluoromethyl)phenyl, 4-(1,1-difluoroethyl)phenyl, 4-(2-methoxyethoxy)phenyl, 2-fluoro-4-methoxyphenyl, 2-fluoro-4-((tetrahydrofuran-3-yl)oxy)phenyl, 4-methoxy-2-methylphenyl, 4-(((tetrahydro-2H-pyran-4-yl)oxy)methyl)phenyl, 4-((oxetan-3-yloxy)methyl)phenyl, 4-(((tetrahydrofuran-3-yl)oxy)methyl)phenyl, 4-((2-methoxyethoxy)methyl)phenyl, 4-(difluoromethoxy)phenyl, 3-fluoro-4-methoxyphenyl, 3-methyl-4-(oxetan-3-yloxy)phenyl, 2-fluoro-4-(oxetan-3-yloxy)phenyl, 3-fluoro-4-(oxetan-3-yloxy)phenyl, 4-((3-methyloxetan-3-yl)oxy)phenyl, 4-((dimethylamino)methyl)-3,5-difluorophenyl, 4-(pyrrolidin-1-ylmethyl)phenyl, 4-((3,3-difluoroazetidin-1-yl)methyl)phenyl, 4-((dimethylamino)methyl)phenyl, 4-((methylamino)methyl)phenyl, 4-(1-methyl-1H-1,2,3-triazol-4-yl)phenyl, 4-isopropylphenyl, 4-cyclopropoxyphenyl, 4-isopropoxyphenyl, 4-methoxyphenyl, 4,5-dimethylthiazol-2-yl, 4,5,6,7-tetrahydrobenzo[d]thiazol-2-yl, 6-cyclopropoxypyridin-3-yl, 6-cyclopropylpyridin-3-yl, 6-cyclopropyl-5-fluoropyridin-3-yl, 6-methoxypyridin-3-yl, 6-(methoxymethyl)pyridin-3-yl, 6-isopropylpyridin-3-yl, 6-ethylpyridin-3-yl, 6-cyclopropyl-2-fluoropyridin-3-yl, 6-(2-fluoropropan-2-yl)pyridin-3-yl, 6-cyclopropyl-4-fluoropyridin-3-yl, 2-cyclopropylpyrimidin-5-yl, 6-methyl-5-(trifluoromethyl)pyridin-2-yl, 5-isopropoxypyridin-2-yl, 5-cyclopropylpyridin-2-yl, 3,5-dimethylpyridin-2-yl, 5,6-dimethylpyridin-2-yl, 5-methoxy-6-methylpyridin-2-yl, 6-(methoxymethyl)-5-methylpyridin-2-yl, 5-cyclopropyl-3-methylpyridin-2-yl, 5-cyclopropyl-6-methylpyridin-2-yl, 5-methoxypyridin-2-yl, 5-methoxy-3-methylpyridin-2-yl, 1-(5-(methoxymethyl)pyridin-2-yl, 5-cyclopropyl-6-fluoropyridin-2-yl, 5-cyclopropyl-4-fluoropyridin-2-yl, 2,3-dihydro-1H-inden-5-yl, 1,1-dimethyl-2,3-dihydro-1H-inden-5-yl, 2,3-dihydrobenzofuran-5-yl, benzo[d][1,3]dioxol-5-yl, 4-fluoro-2,2-dimethylbenzo[d][1,3]dioxol-5-yl, 2,2-dimethylbenzo[d][1,3]dioxol-5-yl, 6,7-dihydro-5H-cyclopenta[b]pyridin-2-yl, 2,3-dihydrofuro[3,2-b]pyridin-5-yl, 2,3-dihydrofuro[2,3-b]pyridin-5-yl, [1,3]dioxolo[4,5-b]pyridin-5-yl, 3-fluoropyrazolo[1,5-a]pyridin-6-yl, pyrazolo[1,5-a]pyridin-6-yl, 3-methyl-3H-imidazo[4,5-b]pyridin-6-yl, 3-methylimidazo[1,2-a]pyridin-7-yl, imidazo[1,2-a]pyridin-7-yl, [1,2,4]triazolo[1,5-a]pyridin-6-yl, imidazo[1,2-a]pyridin-6-yl, imidazo[1,5-a]pyridin-6-yl, benzo[d]oxazol-6-yl, 7-fluoro-2-methylbenzo[d]oxazol-6-yl, 4-acetamido-2-fluoro-3-hydroxyphenyl, benzo[c]isothiazol-5-yl, benzo[b]thiophen-6-yl, benzo[d]thiazol-6-yl, 2-methylbenzo[d]thiazol-6-yl, 2-ethylbenzo[d]thiazol-6-yl, thiazolo[5,4-b]pyridin-5-yl, thiazolo[4,5-b]pyridin-6-yl, 4-fluorobenzo[d]thiazol-6-yl, 7-fluorobenzo[d]thiazol-6-yl, 5-fluorobenzo[d]thiazol-6-yl, 7-fluoro-2-methylbenzo[d]thiazol-6-yl, benzo[d]oxazol-5-yl, benzo[c][1,2,5]thiadiazol-5-yl, benzo[d]thiazol-5-yl, 2-methylbenzo[d]thiazol-5-yl, 2-ethylbenzo[d]thiazol-5-yl, 4-fluorobenzo[d]thiazol-5-yl, 3-methylquinoxalin-2-yl, 5-fluoro-2-methylbenzo[d]thiazol-6-yl, 2-methylthiazolo[5,4-b]pyridin-5-yl, thieno[2,3-b]pyridin-6-yl, 2-methylthieno[2,3-b]pyridin-6-yl, 2-fluorothieno[2,3-b]pyridin-6-yl, 6-(1,1-difluoroethyl)pyridin-3-yl, 4-trifluoromethyl-phenyl, 3-methylquinoxalin-6-yl, or 3-(methyl-d3)quinoxalin-6-yl.

In some embodiments, Cy₁ is 2-methylthiazolo[5,4-b]pyridin-5-yl, 2-(difluoromethyl)thieno[2,3-b]pyridin-6-yl, 2-methylimidazo [1,2-b]pyridazin-6-yl, 2-methylpyrazolo[1,5-a]pyrimidin-5-yl, pyrazolo[1,5-a]pyridine-5-yl, 3-fluoro-2-methylpyrazolo[1,5-a]pyrimidin-5-yl, 3-fluoropyrazolo[1,5-a]pyrimidin-5-yl, 3-fluoropyrazolo[1,5-a]pyridin-5-yl, 3-chloroimidazo[1,2-b]pyridazin-6-yl, or 3-fluoroimidazo[1,2-b]pyridazin-6-yl.

Disclosed herein provides a compound, or a stereoisomer, or a pharmaceutically acceptable salt thereof, wherein said compounds is any one of the exemplified compounds.

Disclosed herein provides a pharmaceutical composition comprising one or more compounds herein, or a stereoisomer or a pharmaceutically acceptable salt thereof, and a pharmaceutically acceptable excipient.

Disclosed herein provides a method of treating cancer, comprising administering to a patient in need of such treatment a therapeutically effective amount of a compound herein or a pharmaceutical composition herein.

### DETAILED DESCRIPTION OF THE DISCLOSURE

### Definitions

The following terms have the indicated meanings throughout the specification:

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups derived from an alkane by removal of one hydrogen atom from the same carbon atom, which comprises from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups. The alkyl group can be optionally enriched in deuterium, e.g., -CD₃, -CD₂CD₃ and the like. The term “alkylene” refers to a hydrocarbon group selected from linear and branched saturated hydrocarbon groups derived from an alkane by removal of two hydrogen atoms from the same carbon atom, which comprises from 1 to 6, such as from 1 to 4, carbon atoms, further such as from 1 to 3, more further such as 1, 2 or 3 carbon atoms, include, but not limited to, methylene (—CH₂—), ethylene (—CH₂CH₂—), 1-methymethylene (—CH(CH₃)—), or trimethylene (—CH₂CH₂CH₂—).

The term “halogen” refers to fluoro (F), chloro (C₁), bromo (Br) and iodo (I).

The term “haloalkyl” refers to an alkyl group in which one or more hydrogen is/are replaced by one or more halogen atoms such as fluoro, chloro, bromo, and iodo. Examples of the haloalkyl include haloC₁₋₈alkyl, haloC₁₋₆alkyl or halo C₁₋₄alkyl, but not limited to —CF₃, —CH₂Cl, —CH₂CF₃, —CCl₂, CF₃, and the like.

The term “alkyloxy” or “alkoxy” refers to an alkyl group as defined above attached to the parent molecular moiety through an oxygen atom. Examples of an alkyloxy, e.g., C₁₋₆alkyloxy or C₁₋₄ alkyloxy include, but not limited to, methoxy, ethoxy, isopropoxy, propoxy, n-butoxy, tert-butoxy, pentoxy and hexoxy and the like.

The term “amino” refers to —NH₂.

The term “alkenyl” herein refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkynyl” herein refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. In particular, Examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “deuterated” is used herein to modify a chemical structure or an organic group or radical, wherein one or more carbon-bound hydrogen(s) are replaced by one or more deuterium(s), e.g., “deuterated-alkyl”, “deuterated-cycloalkyl”, “deuterated-heterocycloalkyl”, “deuterated-aryl”, “deuterated-morpholinyl”, and the like. For example, the term “deuterated-alkyl” defined above refers to an alkyl group as defined herein, wherein at least one hydrogen atom bound to carbon is replaced by a deuterium. In a deuterated alkyl group, at least one carbon atom is bound to a deuterium; and it is possible for a carbon atom to be bound to more than one deuterium; it is also possible that more than one carbon atom in the alkyl group is bound to a deuterium.

The term “aryl” used alone or in combination with other terms refers to a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “heteroaryl” herein refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

The term “optionally oxidized sulfur” used herein refers to —S—, SO or SO₂.

The terms “aromatic heterocyclic ring” and “heteroaryl” are used interchangeably throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is a 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) 1H-pyrazolyl (such as 1H-pyrazol-3-yl, 1H-pyrazol-4-yl or 1H-pyrazol-5-yl), pyridyl or pyridinyl (such as 2-pyridyl, 3-pyridyl, or 4-pyridyl), cinnolinyl, pyrazinyl, pyrimidinyl (such as pyrimidin-2-yl, pyrimidin-4-yl, pyrimidin-5-yl or 2,4-pyrimidinyl, 3,5-pyrimidinyl), imidazolyl (such as 1H-imidazol-2-yl, 1H-imidazol-4-yl, 1H-imidazol-5-yl, or 2,4-imidazolyl), imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl (such as 1H-indol-2-yl, 1H-indol-3-yl, 1H-indol-4-yl, 1H-indol-5-yl, 1H-indol-6-yl or 1H-indol-7-yl), isoindolyl, indolinyl, oxadiazolyl (such as 1,2,3-oxadiazolyl, 1,2,4-oxadiazolyl, or 1,3,4-oxadiazolyl), phthalazinyl, pyrazinyl (such as pyrazin-2-yl), pyridazinyl, pyrrolyl, triazolyl (such as 1,2,3-triazolyl, 1,2,4-triazolyl, or 1,3,4-triazolyl), quinolinyl (such as quinolin-2-yl, quinolin-3-yl, quinolin-4-yl, quinolin-5-yl, quinolin-6-yl, or quinolin-7-yl), isoquinolinyl (such as isoquinolin-1-yl, isoquinolin-3-yl, isoquinolin-4-yl, isoquinolin-5-yl, isoquinolin-6-yl, isoquinolin-7-yl, or isoquinolin-8-yl), pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl (such as furazan-2-yl, furazan-3-yl), benzofurazanyl, benzothiophenyl, benzothiazolyl, benzoxazolyl (such as benzo[d]oxazol-2-yl, benzo[d]oxazol-4-yl, benzo[d]oxazol-5-yl, benzo[d]oxazol-6-yl or benzo[d]oxazol-7-yl), quinazolinyl, quinoxalinyl (such as quinoxalin-2-yl, quinoxalin-3-yl, quinoxalin-4-yl, quinoxalin-5-yl, quinoxalin-6-yl, quinoxalin-7-yl or quinoxalin-8-yl), naphthyridinyl (such as 1,8-naphthyridin-2-yl, 1,8-naphthyridin-3-yl, or 1,8-naphthyridin-4-yl), 2,3-dihydro-[1,4]dioxino[2,3-b]pyridinyl (such as 2,3-dihydro-[1,4]dioxino[2,3-b]pyridin-6-yl, 2,3-dihydro-[1,4]dioxino[2,3-b]pyridin-7-yl, or 2,3-dihydro-[1,4]dioxino[2,3-b]pyridin-8-yl), furopyridinyl, benzothiazolyl (such as benzo[d]thiazol-2-yl, benzo[d]thiazol-4-yl, benzo[d]thiazol-5-yl, benzo[d]thiazol-6-yl or benzo[d]thiazol-7-yl), benzo[d]imidazolyl (such as 1H-benzo[d]imidazol-2-yl, 1H-benzo[d]imidazol-4-yl, 1H-benzo[d]imidazol-5-yl, 1H-benzo[d]imidazol-6-yl or 1H-benzo[d]imidazol-7-yl), [1,2,4]triazolo[1,5-a]pyridinyl (such as [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, or [1,2,4]triazolo[1,5-a]pyridin-8-yl), 3H-imidazo[4,5-b]pyridinyl (such as 3H-imidazo[4,5-b]pyridin-2-yl, 3H-imidazo[4,5-b]pyridin-5-yl, 3H-imidazo[4,5-b]pyridin-6-yl or 3H-imidazo[4,5-b]pyridin-7-yl), 1H-imidazo[4,5-b]pyridinyl (such as 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl), [1,2,4]triazolo[1,5-a]pyridinyl (such as [1,2,4]triazolo[1,5-a]pyridin-2-yl, 1,2,4]triazolo[1,5-a]pyridin-5-yl, [1,2,4]triazolo[1,5-a]pyridin-6-yl, [1,2,4]triazolo[1,5-a]pyridin-7-yl or [1,2,4]triazolo[1,5-a]pyridin-8-yl), indazolyl (such as 1H-indazol-5-yl) and 5,6,7,8-tetrahydroisoquinoline.

Also, a “heteroaryl” fused with a “Heterocyclyl” is defined as “heteroaryl”.

“Heterocyclyl,” “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “monocyclic heterocyclyl” refers to monocyclic groups in which at least one ring member is a heteroatom selected from nitrogen, oxygen or optionally oxidized sulfur. A heterocycle may be saturated or partially saturated.

Exemplary monocyclic 4 to 9-membered heterocyclyl groups include, but not limited to, (as numbered from the linkage position assigned priority 1) pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2,5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, or 1,1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/4-membered, 3-membered/5-membered, 4-membered/5-membered, 4-membered/6-membered, 5-membered/5-membered, or 5-membered/6-membered mono-spiro heterocyclyl.

The term “fused heterocyclic group” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but none of the rings has a completely conjugated pi-electron system. Preferably, a fused heterocyclyl is 6 to 14-membered, and more preferably 7 to 10-membered. According to the number of membered rings, a fused heterocyclyl is divided into bicyclic, tricyclic, tetracyclic, or polycyclic fused heterocyclyl, preferably refers to bicyclic or tricyclic fused heterocyclyl, and more preferably 5-membered/5-membered, or 5-membered/6-membered bicyclic fused heterocyclyl. Representative examples of fused heterocycles include, but not limited to, the following groups octahydrocyclopenta[c]pyrrole (e.g., octahydrocyclopenta[c]pyrrol-2-yl), octahydropyrrolo[3,4-c]pyrrolyl, octahydroisoindolyl, isoindolinyl (e.g., isoindoline-2-yl), octahydro-benzo[b][1,4]dioxin.

The term “bridged heterocyclyl” refers to a 5- to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl is divided into bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include, but not limited to, the following groups: 2-azabicyclo[2.2.1]heptyl, azabicyclo[3.1.0]hexyl, 2-azabicyclo[2.2.2]octyl and 2-azabicyclo[3.3.2]decyl.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the titled stereoisomer contains no more than 35%, such as no more than 30%, further such as no more than 25%, even further such as no more than 20%, by weight of any other stereoisomer(s). In some embodiments, the term “substantially pure” means that the titled stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or flash column chromatography. Flash column chromatography can involve any number of methods including, for example: reverse-phase and normal phase; size exclusion; ion exchange; high, medium and low pressure liquid flash column chromatography methods and apparatus; small scale analytical; simulated moving bed (“SMB”) and preparative thin or thick layer flash column chromatography, as well as techniques of small scale thin layer and flash column flash column chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by flash column chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents [Eliel, E. and Wilen, S. Stereochemistry of Organic Compounds. New York: John Wiley & Sons, Inc., 1994; Lochmuller, C. H., et al. “Flash column chromatographyic resolution of enantiomers: Selective review.” J. Chromatogr., 113 (3) (1975): pp. 283-302]. Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993. The absolute configuration of the chiral centers in a compound can be determined using methods known to one skilled in the art, e.g., single crystal X-ray crystallography or co-crystal formation of a compound of interest with the targeted proteins, sometime coupled with a spectroscopic technique, e.g., NMR spectroscopy. In some embodiments, the absolute configuration of chiral centers in a compound can be elucidated from the X-ray single-crystal structure of the compound. In some embodiments, the absolute configuration of chiral centers elucidated by the X-ray crystal structure of a compound can be used to infer the absolute configuration of the corresponding chiral centers in another compound or an intermediate obtained from the same or similar synthetic methodologies.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

“Selective inhibitory activity” or “selectivity” refers to the difference in the degree of inhibition against DGKα and DGKζ; the greater the degree of inhibition effected for a particular isoform relative to another isoform, the greater the selectivity the inhibitor exhibits for that particular isoform. In some embodiments, “a compound showing selective inhibitory activity of DGKα over DGKζ” refers a compound which shows an IC50 against DGKα is not larger than about 2000 nM with the ratio of IC50 against DGK ζ and IC50 against DGKα larger than or equal to about 20; “a compound showing selective inhibitory activity of DGKζ over DGKα” refers a compound which shows an IC50 against DGKζ is not larger than about 2000 nM with the ratio of IC50 against DGKα and IC50 against DGKζ larger than or equal to about 20; and “a compound showing dual inhibitory activity” refers to a compound which shows inhibitory activities against both DGKα and DGKζ with IC50 no larger than 500 nM and the ratio of the two IC50 values no more than 20.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” include salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid Formulation such as tablets, powder, granule, capsules and the like, a liquid Formulation such as water or oil suspension or other liquid Formulation such as syrup, solution, suspension or the like; for parenteral administration, the pharmaceutical composition may be solution, water solution, oil suspension concentrate, lyophilized powder or the like. Preferably, the Formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All Formulations of the pharmaceutical composition disclosed herein can be produced by the conventional methods in the pharmaceutical field. For example, the active ingredient can be mixed with one or more excipients, then to make the desired Formulation. The “pharmaceutically acceptable excipient” refers to conventional pharmaceutical carriers suitable for the desired pharmaceutical Formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the claims which follow, unless the context requires otherwise, the term “comprise,” and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cn-m” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### Abbreviations

| | |
|---|---|
| Ac | Acetyl |
| AcOH | Acetic acid |
| Aq | Aqueous |
| Brine | Saturated aqueous sodium chloride solution |
| Bn | Benzyl |
| Boc | Tert-butyloxycarbonyl |
| Cbz | Benzyloxycarbonyl |
| CDI | N,N′-Carbonyldiimidazole |
| DMF | N,N-Dimethylformamide |
| Dppf | 1,1″-bis(diphenylphosphino)ferrocene |
| DBU | 1,8-diazabicyclo[5.4.0]undec-7-ene |
| DCE | 1,2-dichloroethane |
| DCM | Dichloromethane |
| DHP | 3,4-dihydro-2H-pyran |
| DIPEA | N,N-diisopropylethylamine |
| DMAP | 4-N,N-dimethylaminopyridine |
| DMB | 2,4-dimethoxybenzyl |
| DMB-Cl | 1-(chloromethyl)-2,4-dimethoxybenzene |
| DMF | N,N-dimethylformamide |
| DMSO | Dimethyl sulfoxide |
| EA | Ethyl acetate |
| eq | Equivalent |
| g | Grams |
| HATU | O-(7-Azabenzotriazol-1-yl)-N,N,N′,N′- |
| | tetramethyluronium hexafluorophosphate |
| HCl | Hydrochloric acid |
| HPLC | High-performance liquid flash column chromatography |
| IPA | Isopropyl alcohol |
| i-PrOH | Isopropyl alcohol |
| KHMDS | Potassium Bis(trimethylsilyl)amide |
| LDA | Lithium diisopropylamide |
| LiHMDS | Lithium bis(trimethylsilyl)amide |
| m-CPBA | 3-Chloroperoxybenzoic acid |
| mg | Milligrams |
| mL | Milliliters |
| mmol | Millimole |
| MeCN | Acetonitrile |
| MeOH | Methanol |
| Min | Minutes |
| MS | Mass spectrum |
| n-BuLi | n-Butyllithium |
| NMR | Nuclear magnetic resonance |
| NCS | N-Chlorosuccinimide |
| NBS | N-Bromosuccinimide |
| NaHMDS | Sodium bis(trimethylsilyl)amide |
| OTf | Trifluoromethanesulfonate |
| Pd/C | Palladium on carbon |
| Pd(dppf)Cl₂ | [1,1′-Bis(diphenylphosphino)ferrocene]dichloro- |
| | palladium(II) |
| PE | Petroleum ether |
| PMB | 4-Methoxybenzyl |
| PMB-Cl | 1-(chloromethyl)-4-methoxybenzene |
| RT | room temperature |
| SEM | 2-trimethylsilylethoxymethoxy(2- |
| | (chloromethoxy)ethyl)trimethylsilane |
| TBSCl | tert-Butyldimethylsilyl chloride |
| TBAB | Tetrabutylammonium bromide |
| TBAF | Tetrabutylammonium fluoride |
| TBDMSCl | tert-Butyldimethylsilyl chloride |
| tBuXPhos | Bis(1,1-dimethylethyl)[2′,4′,6′-tris(1- |
| | methylethyl)[1,1′-biphenyl]-2-yl]phosphine |
| tBuXPhos Pd G3 | Methanesulfonato(2-di-t-butylphosphino-2′,4′,6′-tri-i- |
| | propyl-1,1′-biphenyl)(2′-amino-1,1′-biphenyl-2- |
| | yl)palladium(II) |
| t-BuOK | Potassium tert-butoxide |
| TEA | Triethanolamine |
| TFA | Trifluoroacetic acid |
| THF | Tetrahydrofuran |
| THP | tetrahydropyran3,4-dihydro-2H-pyran |
| TLC | thin layer flash column chromatography |
| TMSCN | Trimethylsilyl cyanide |
| TMSOTf | Trimethylsilyl trifluoromethanesulfonate |
| XPhos | 2-Dicyclohexylphosphino-2′,4′,6′-triisopropylbiphenyl |

### General Synthetic Schemes

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from room temperature to the solvent's boiling temperature. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica flash column chromatography.

Chiral analytic HPLC was used for the retention time analysis of different chiral examples, the conditions were divided into the methods as below according to the column, mobile phase, solvent ration used. Preparation of homochiral examples may be carried out by techniques known to one skilled in the art. The absolute stereochemistry was not assigned at the newly formed carbon-nitrogen bond.

The compounds disclosed herein can be prepared by following Scheme I to III.

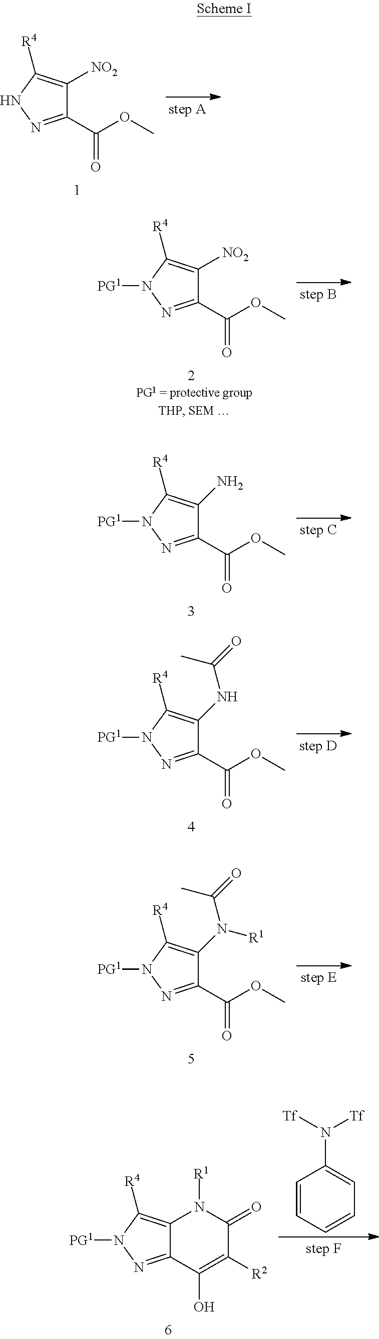

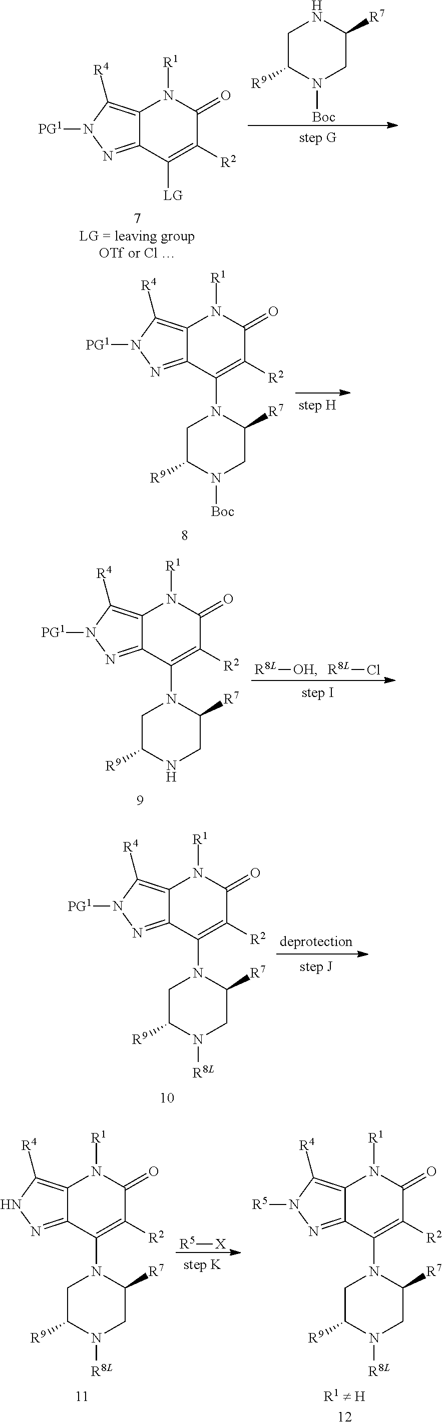

wherein the substitutions from R¹ to R⁹ and R^{8L} (corresponding to L¹-Cy¹) are as defined as mentioned in Formula (I).

In scheme I, a commercially available Compound 1 is protected with THP or SEM to give Compound 2. Compound 2 is reduced under reducing conditions (such as Pd—C/H₂, or Fe powder/NH₄Cl) to form Compound 3 as an amine. Compound 3 is acetylated by acetyl chloride or acetic anhydride to give Compound 4, and then reacted with appropriate R¹—X/R¹-OTf under basic condition (such as Cs₂CO₃, NaH, or t-BuOK) to give Compound 5. Compound 5 is further cyclized under basic condition (such as LiHMDS, NaHMDS, or KHMDS) to give Compound 6. Compound 6 is reacted with trifluoromethulfonate reagent (such as Tf₂O, or PhNTf₂) or phosphoryl chloride to give Compound 7. Compound 7 is reacted with the appropriate chiral secondary amine by nucleophilic aromatic substitution reaction to give Compound 8. The protected groups on the amine of Compound 8 are removed to give Compound 9 under acid condition (such as TFA or 4M solution of HCl in 1,4-dioxane). Tertiary amines Compound 10 is prepared by N-alkylation of secondary amines compound 9 by treatment with alkylating agents (such as alkylhalides or sulfonates) or reductive alkylation with aldehydes or ketones, the most frequently used procedures via a phosphonium salt mediated alkylation of amines with corresponding alcohols (Florencio Zaragoza and Henrik Stephensen, J. Org. Chem. 2001, 66, 2518-2521). Compound 10 is deprotected using acid condition (such as TFA or 4M solution of HCl in 1,4-dioxane) to give Compound 11. Routine reaction methods, such as alkylation, alkenylation reaction, acylation reaction or sulfonylation reaction of Compound 11 with corresponding alkenyl borate using copper-catalyzed Chan-Lam reaction or corresponding alkyl halides under basic condition (such as Cs₂CO₃, NaH, or t-BuOK), corresponding alkyl sulfonyl chloride/aliphatic acyl chloride using condensation reaction condition (such as Et₃N/HATU) to afford a compound of Formula 12 (wherein R² is H), and the halogenation of the compound of Formula 12 (wherein R² is H) with electrophilic halogenating reagents (such as NBS, NCS, or selectfluor) to afford a compound of Formula 12 (wherein R² is F, Cl, or Br), then the compound of Formula I (wherein R² is Br) also can be used to produce a compound of Formula 12 (wherein R² is CN, or Me) by normally Pd-catalyst coupling reaction with Zn(CN)₂ and 2,4,6-Trimethylboroxin.

Compound 10d disclosed herein can also be prepared by the following Scheme II.

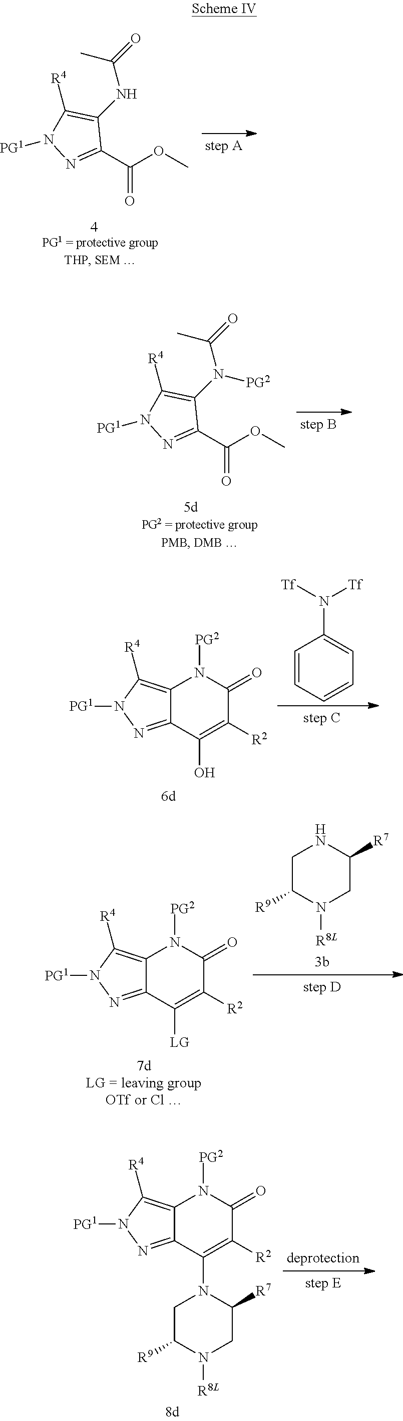

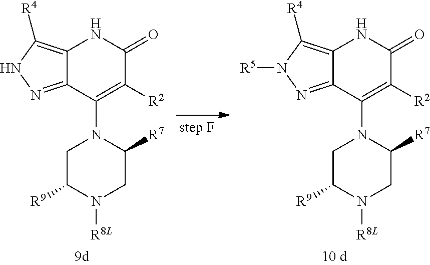

In Scheme II, installation of PG² proceeded through alkylation reaction of compound 4 with appropriate R¹X (such as PMB-Cl, or DMB-Cl) under basic condition (such as Cs₂CO₃, or NaH) to give Compound 5d; which is cyclized under basic condition (such as lithium bis(trimethylsilyl)amide, sodium bis(trimethylsilyl)amide, potassium bis(trimethylsilyl)amide . . . ) to give Compound 6d, which subsequently react with trifluoromethulfonate reagent (such as trifluoromethanesulfonic anhydride, N-phenylbis(trifluoromethanesulfon)imide) or phosphoryl chloride to give Compound 7d, which is reacted with the corresponding secondary amine 3b by nucleophilic aromatic substitution reaction to give Compound 8d. The protective groups of Compound 8d are removed under strong acid condition (such as TfOH, or TFA) to give Compound 9d, which undergoes selective N-alkylation on pyrazole of Compound 9d by treatment with corresponding alkylhalides providing the compound of Formula 10d.

The compound of Formula 10A disclosed herein can also be prepared by following Scheme III.

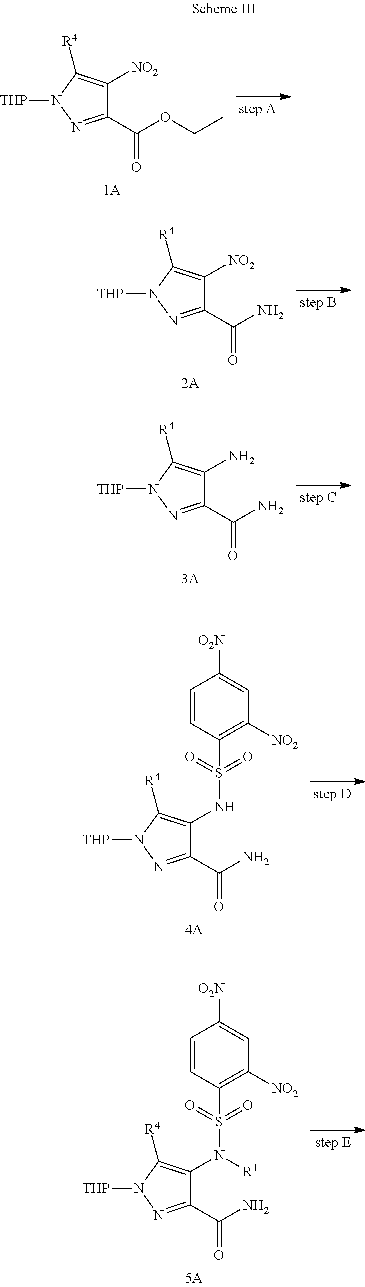

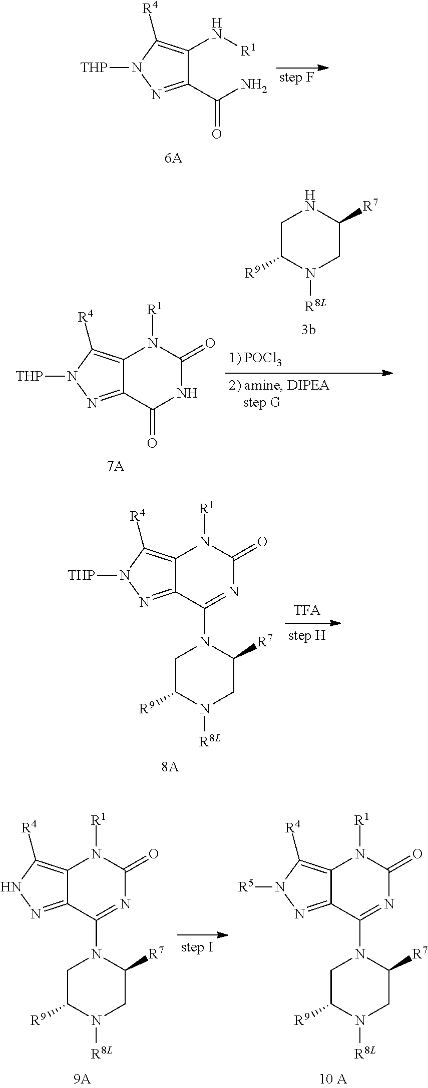

In Scheme Ill, commercially available Compound 1A via aminolysis reaction to afford Compound 2A which undergoes reduction providing Compound 3A with an appropriate reducing condition (such as Pd—C/H₂, or Fe powder/NH₄Cl). Compound 3A subsequently react corresponding sulfonyl chloride under basic condition (such as Et₃N, or pyridine) to afford Compound 4A, which react with appropriate R¹X/R¹OTf under basic condition (such as K₂CO₃, Cs₂CO₃, or NaH) to give Compound 5A. The protected group on the amine of Compound 5A is removed by 2-mercaptoacetic acid to give Compound 6A. Treatment of Compound 6A with 1,1′-carbonyldiimidazole under basic condition (such as NaH) afford cyclization Compound 7A, installation of the amine moiety 3b proceed through chlorination with phosphoryl chloride to give the chlorinated intermediate which is reacted with 3b to prevent hydrolysis giving Compound 8A, which is deprotected using acid condition (such as TFA or 4M solution of HCl in 1,4-dioxane) to give Compound 9A, and alkylation reaction of Compound 9A with corresponding alkyl halides under basic condition (such as Cs₂CO₃, or NaH) to afford the compound of Formula 10A.

### EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for flash column chromatography and silica gel (GF254) for thin-layer flash column chromatography (TLC) are commercially available from Tsingdao Haiyang Chemical Co., Ltd. or Yantai Chemical Co., Ltd. of China; all are eluted with petroleum ether (60-90° C.)/ethyl acetate (v/v), and visualized by iodine or the solution of molybdphosphoric acid in ethanol unless otherwise specified. All extraction solvents, unless otherwise specified, are dried over anhydrous Na₂SO₄.

¹H NMR spectra are recorded on Bruck-400 nuclear magnetic resonance spectrometer with TMS (tetramethylsilane) as the internal standard. LC/MS data are recorded by using Agilent1100 High Performance Liquid Flash column chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw®.

### Synthesis

### Preparative HPLC Conditions (Method A)

| | |
|---|---|
| Column | Phenomenex Gemini NX-C18, 150 × 21.2 mm, 5 μm |
| Column Temp. | R.T. |
| Detection | DAD, UV λ = 214/254 nm |
| Wavelength | |
| Run Time | 17.0 min |
| Flow Rate | 20.0 mL/min |
| Mobile Phase A | 0.1% FA-H₂O (v/v) |
| Mobile Phase B | 0.1% FA-CH₃CN (v/v) |

### Preparative HPLC Conditions (Method B)

| | |
|---|---|
| Column | Waters XSelect CSH C18, 150 × 19 mm, 5 μm |
| Column Temp. | R.T. |
| Detection Wavelength | DAD, UV λ = 214/254 nm |
| Run Time | 17.0 min |
| Flow Rate | 17 mL/min |
| Mobile Phase A | 0.03% NH₃•H₂O—H₂O (v/v) |
| Mobile Phase B | CH₃CN |

### Intermediate 1: 4-methyl-5-oxo-2-(tetrahydro-2H-pyran-2-yl)-4,5-dihydro-2H-pyrazolo[4,3-b]pyridin-7-yl trifluoromethanesulfonate

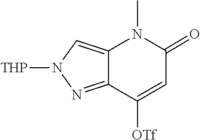

### Step A: methyl 4-nitro-1-(tetrahydro-2H-pyran-2-yl)-1H-pyrazole-3-carboxylate

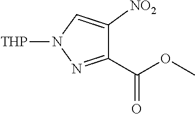

To a solution of methyl 4-nitro-1H-pyrazole-3-carboxylate (85.5 g, 0.5 mol) and TsOH·H₂O (9.5 g, 0.05 mol) in THF (0.6 L) was added DHP (84 g, 1 mol) in some portions. The reaction was stirred at 80° C. for 16 hours. Then the mixture was cooled to room temperature and concentrated to dryness. The resulting residue was purified by flash column chromatography to give the titled compound (121 g, 94%). ¹H NMR (400 MHz, CDCl₃): δ 8.38 (s, 1H), 5.43 (dd, J=2,4, 8.8 Hz, 1H), 4.15-4.05 (m, 1H), 3.99 (s, 3H), 3.78-3.68 (m, 1H), 2.26-2.17 (m, 1H), 2.01-1.85 (m, 2H), 1.76-1.60 (m, 3H) ppm. MS: M/e 278 (M+23)⁺.

### Step B: methyl 4-amino-1-(tetrahydro-2H-pyran-2-yl)-1H-pyrazole-3-carboxylate

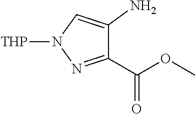

To a solution of methyl 4-nitro-1-(tetrahydro-2H-pyran-2-yl)-1H-pyrazole-3-carboxylate (64 g & 57 g, two batches) in MeOH (300 mL) was added (6 g). The mixture was stirred at room temperature under H₂ atmosphere, filtered and washed with MeOH. The combined filtrates were concentrated to give the titled compound (96 g, 90%). ¹H NMR (400 MHz, CDCl₃): δ 7.21 (s, 1H), 5.33 (dd, .=2,4, 9.6 Hz, 1H), 4.11-4.02 (m, 1H), 3.92 (s, 3H), 3.71-3.62 (m, 1H), 2.12-1.90 (m, 3H), 1.76-1.52 (m, 3H) ppm. MS: M/e 226 (M+1)⁺.

### Step C: methyl 4-acetamido-1-(tetrahydro-2H-pyran-2-yl)-1H-pyrazole-3-carboxylate

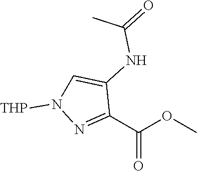

To a solution of methyl 4-amino-1-(tetrahydro-2H-pyran-2-yl)-1H-pyrazole-3-carboxylate (96 g, 426 mmol) in pyridine (0.4 L) was added Ac₂O (60 mL) dropwise to keep the temperature below 20° C. The resulting mixture was stirred at RT for 3 hours and concentrated to dryness. The resulting residue was recrystallized from PE/EA to give the titled compound (84 g, 74%). ¹H NMR (400 MHz, CDCl₃): δ 8.99 (s, 1H), 8.43 (s, 1H), 5.46-5.36 (m, 1H), 4.12-4.03 (m, 1H), 3.96 (s, 3H), 3.74-3.60 (m, 1H), 2.20 (s, 3H), 2.14-1.96 (m, 3H), 1.75-1.54 (m, 3H) ppm. MS: M/e 268 (M+1)⁺.

### Step D: methyl 4-(N-methylacetamido)-1-(tetrahydro-2H-pyran-2-yl)-H-pyrazole-3-carboxylate

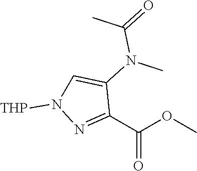

To a solution of methyl 4-acetamido-1-(tetrahydro-2H-pyran-2-yl)-1H-pyrazole-3-carboxylate (84 g, 314.6 mmol) in DMF (0.5 L) was added Cs₂CO₃ (306 g, 943 mmol), followed by CH₃I (134 g, 943 mmol). The reaction was stirred at room temperature (RT) for 16 hours. The reaction mixture was filtered through Celite, washed with DCM. The filtrate was concentrated, and then the obtained residue was diluted with water, extracted with DCM (400 mL×2), washed with brine, dried over Na₂SO₄, filtered, and concentrated to give the titled compound (92 g, crude). ¹H NMR (400 MHz, CDCl₃): δ 7.67 (s, 1H), 5.47-5.36 (m, 1H), 4.16-4.03 (m, 1H), 3.92 (s, 3H), 3.77-3.65 (m, 1H), 3.18 (s, 3H), 2.23-2.12 (m, 1H), 2.09-1.90 (m, 2H), 1.87 (s, 3H), 1.80-1.56 (m, 3H) ppm MS: M/e 282 (M+1)⁺.

### Step E: 7-hydroxy-4-methyl-2-(tetrahydro-2H-pyran-2-yl)-2,4-dihydro-5H-pyrazolo[4,3-b]pyridin-5-one

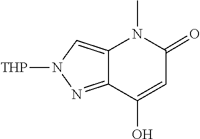

To a solution of methyl 4-(N-methylacetamido)-1-(tetrahydro-2H-pyran-2-yl)-1H-pyrazole-3-carboxylate (88 g, 313 mmol) in THF (0.8 L) was added dropwise a solution of LiHMDS (−500 mL, 1 mol/L, -1.6 eq) at −70° C. (A suspension was formed). The reaction was stirred for 0.5 hour, quenched with water, and warmed to RT slowly. The resulting mixture was extracted with EA. The water phase was collected, treated with citric acid to pH 3-4, and stirred at RT for 0.5 hours to give a suspension. The suspension was filtered, dried to give the titled compound (45.5 g). ¹H NMR (400 MHz, DMSO-d₆) δ 11.24 (s, 1H), 8.07 (s, 1H), 5.65 (s, 1H), 5.51 (dd, J=2,4 Hz, 10.0 Hz, 1H), 4.00-3.88 (m, 1H), 3.72-3.61 (m, 1H), 3.31 (s, 3H), 2.19-2.05 (m, 1H), 2.03-1.89 (m, 2H), 1.80-1.64 (m, 1H), 1.61-1.50 (m, 2H) ppm. MS: M/e 250 (M+1)⁺.

### Step F: 4-methyl-5-oxo-2-(tetrahydro-2H-pyran-2-yl)-4,5-dihydro-2H-pyrazolo[4,3-b]pyridin-7-yl trifluoromethanesulfonate (Intermediate 1)

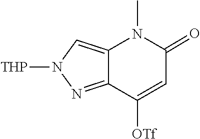

To a solution of 7-hydroxy-4-methyl-2-(tetrahydro-2H-pyran-2-yl)-2,4-dihydro-5H-pyrazolo[4,3-b]pyridin-5-one (49.8 g, 200 mmol) in THF (0.5 L) was added K₂CO₃ (55 g, 400 mmol), followed by 1,1,1-trifluoro-N-phenyl-N-((trifluoromethyl)sulfonyl)methanesulfonamide (100 g, 943 mmol). The reaction was stirred at room temperature for 16 hours. The reaction mixture was filtered through Celite, and washed with EA. The filtrate was concentrated under reduced pressure. The resulting residue was diluted with water, washed with brine, dried by Na₂SO₄, filtered and concentrated to dryness. The resulting residue was purified by flash column chromatography and furtherly subjected to slurry with EA/PE to give the titled compound (70 g, 90%). ¹H NMR (400 MHz, CDCl₃) δ 7.65 (s, 1H), 6.58 (s, 1H), 5.58 (dd, J=2.8 Hz, 8.0 Hz, 1H), 4.11-4.00 (m, 1H), 3.82-3.70 (m, 1H), 3.52 (s, 3H), 2.26-1.96 (m, 3H), 1.81-1.61 (m, 3H) ppm. MS: M/e 382 (M+1)⁺.

### Intermediate 2: 7-((2S,5R)-2,5-diethylpiperazin-1-yl)-4-methyl-2-(tetrahydro-2H-pyran-2-yl)-2,4-dihydro-5H-pyrazolo[4,3-b]pyridin-5-one

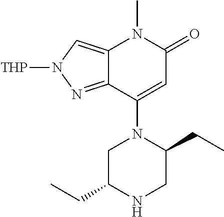

### Step A: methyl (R)-2-(benzylamino)butanoate

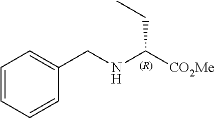

To a solution of methyl (R)-2-aminobutanoate (100.0 g, 0.85 mol) in CH₃CN (1000 mL) was added benzyl bromide (146.1 g, 0.85 mol) at 0° C. under N₂ atmosphere. The reaction was stirred at room temperature overnight and concentrated under reduced pressure. The resulting residue was dissolved in EA (1000 mL) and washed with water (1000 mL×3). The organic layers were concentrated under reduced pressure. The resulting residue was purified by flash column chromatography (PE:EA=10:1) to give the titled compound (106 g, 60%). MS: M/e 208 (M+1)⁺.

### Step B: methyl (R)-2-((S)—N-benzyl-2-((tert-butoxycarbonyl)amino)butanamido)butanoate

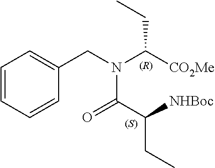

To a solution of methyl (R)-2-(benzylamino)butanoate (117.0 g, 0.56 mol), (S)-2-((tert-butoxycarbonyl)amino)butanoic acid (170.5 g, 0.84 mmol) and 4-Methylmorpholine (113.1 g, 1.12 mmol) in DCM (2000 mL) was added HATU (319.0 g, 0.84 mmol) at 0° C. The reaction was stirred at room temperature overnight and quenched by water and washed with water (1500 mL×2). The organic layers were concentrated under reduced pressure. The resulting residue was purified by flash column chromatography (PE:EA=10:1) to give the titled compound product (178 g, 80%). MS: M/e 393 (M+1)⁺.

### Step C: methyl (R)-2-((S)-2-amino-N-benzylbutanamido)butanoate

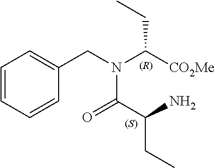

To a solution of methyl (R)-2-((S)—N-benzyl-2-((tert-butoxycarbonyl)amino)butanamido)butanoate (178 g, 0.45 mol) in 1,4-dioxane (100 mL) was added HCl (400 mL, 4 M in 1,4-dioxane) at room temperature. The resulting mixture was stirred at room temperature for another 2 hours, and concentrated under vacuum to give the crude product (200 g, crude). MS: M/e 293 (M+1)⁺.

### Step D: (3S,6R)-1-benzyl-3.6-diethylpiperazine-2,5-dione

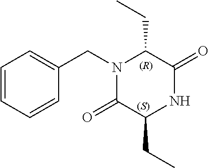

To a solution of methyl (R)-2-((S)-2-amino-N-benzylbutanamido)butanoate (200 g, crude) in EA (1000 mL) was added aq. NaHCO₃ (300 mL) at room temperature. The reaction mixture was stirred at room temperature for another 2 hours. The organic layers were concentrated under reduced pressure. The resulting residue was triturated with MTBE to give the titled compound (61 g, 52% for 2 steps, ee: 97%).MS: M/e 261 (M+1)⁺.

### Step E: (2R,5S)-1-benzyl-2,5-diethylpiperazine

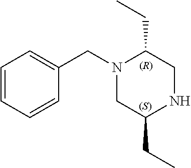

To a solution of LiAlH4 (26.5 g, 0.69 mol) in THF (1000 mL) was added slowly (3S,6R)-1-benzyl-3,6-diethylpiperazine-2,5-dione (61.0 g, 0.23 mol) in THF (500 mL) at 0° C. The resulting mixture was stirred at room temperature for 2 hours, then stirred at 80° C. for overnight. The reaction was quenched by water (27 mL) slowly at 0° C. Then, 1N aqueous NaOH solution (54 mL) and water (81 mL) was added sequentially. The resulting mixture was stirred for 2 hours. The white precipitates that formed was removed by filtration. The filter cake was washed with EA (500 mL). The combined filtrates were evaporated. The resulting residue was dissolved in toluene. The solvent was removed under vacuum to dryness to afford the titled compound (51 g, 95%). MS: M/e 233 (M+1)⁺.

### Step F: 7-((2S,5R) 4-benzyl-2,5-diethylpiperazin-1-yl-4-methyl-2-(tetrahydro-2H-pyran-2-yl)-2,4-dihydro-5H-pyrazolo[4,3-b]pyridin-5-one

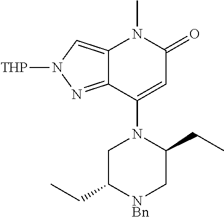

A mixture of 4-methyl-5-oxo-2-(tetrahydro-2H-pyran-2-yl)-4,5-dihydro-2H-pyrazolo[4,3-b]pyridin-7-yl trifluoromethanesulfonate (3.8 g, 10 mmol), (2R,5S)-1-benzyl-2,5-diethylpiperazine (3.6 g, 15 mmol) and DIPEA (6.4 g, 50 mmol) in CH₃CN (50 mL) was heated to 105° C. for overnight under N₂ atmosphere. The solvent was removed under vacuum. The crude product was purified by flash column chromatography (DCM:MeOH=15:1) to give the titled compound (4.2 g, 90%). MS: We 464 (M+1)⁺.

### Step G: 7-((2S,5R)-2,5-diethylpiperazin-1-yl)-4-methyl-2-(tetrahydro-2H-pyran-2-yl)-2,4-dihydro-5H-pyrazolo[4,3-b]pyridin-5-one (Intermediate 2)

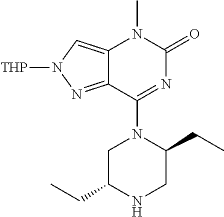

A mixture of 7-((2S,5R)-4-benzyl-2,5-diethylpiperazin-1-yl)-4-methyl-2-(tetrahydro-2H-pyran-2-yl)-2,4-dihydro-5H-pyrazolo[4,3-b]pyridin-5-one (4.2 g, 9.1 mmol) and Pd/C (420 mg, 10% in water) in MeOH (50 mL) was shaken under N₂ atmosphere (50 psi) at room temperature for overnight. The reaction mixture was filtered through Celite. The filter cake was washed with EA (50 mL). The combined filtrates were concentrated to dryness to give the titled compound (3.2 g, 94%). MS: M/e 374 (M+1)⁺.

### Intermediate 3: 2-(7-((2S,5R)-2,5-diethylpiperazin-1-yl)-4-methyl-5-oxo-4,5-dihydro-2H-pyrazolo[4,3-b]pyridin-2-yl)acetonitrile

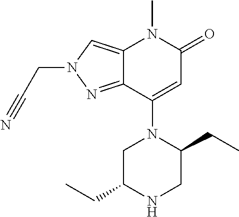 [SMILES: CC[C@@H]1CN(c2cc(=O)n(C)c3cn(CC#N)nc23)[C@@H](CC)CN1]

### Step A: tert-butyl (2R,5S)-2,5-diethyl-4-(4-methyl-5-oxo-4,5-dihydro-2H-pyrazolo[4,3-b]pyridin-7-yl)piperazine-1-carboxylate

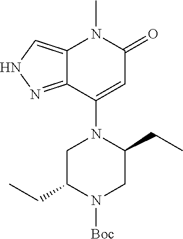 [SMILES: CC[C@@H]1CN(c2cc(=O)n(C)c3c[nH]nc23)[C@@H](CC)CN1C(=O)OC(C)(C)C]

To a stirred solution of 7-((2S,5R)-2,5-diethylpiperazin-1-yl)-4-methyl-2-(tetrahydro-2H-pyran-2-yl)-2,4-dihydro-5H-pyrazolo[4,3-b]pyridin-5-one (3 g, 8.04 mmol) in MeOH (20 mL) was added HCl (5 mL, 4.0 M in 1,4-dioxane). The reaction mixture was stirred over weekend, concentrated to give a residue, which was treated with THF/H₂O (50 mL/20 mL, v/v), K₂CO₃ (3.3 g, 24.1 mmol) was added, followed by Boc₂O (1.75 g, 8.04 mmol). Then the mixture was stirred for 1 hour, acidified to pH=4-5 with citric acid, extracted with EA (30 mL×3). The combined organic layers were washed with brine, dried over Na₂SO₄ and concentrated to dryness. The resulting residue was purified by flash column chromatography to give the titled compound (1.4 g, 45%). MS: M/e 390 (M+1)⁺.

### Step B: tert-butyl (2R,5S)-4-(2-(cyanomethyl)-4-methyl-5-oxo-4.5-dihydro-2H-pyrazolo[4,3-b]pyridin-7-yl)-2,5-diethylpiperazine-1-carboxylate

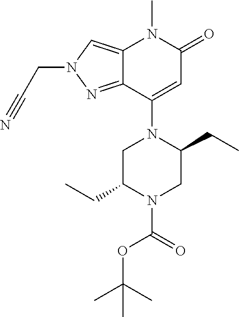 [SMILES: CC[C@@H]1CN(c2cc(=O)n(C)c3cn(CC#N)nc23)[C@@H](CC)CN1C(=O)OC(C)(C)C]

To a stirred solution of tert-butyl (2R,5S)-2,5-diethyl-4-(4-methyl-5-oxo-4,5-dihydro-2H-pyrazolo[4,3-b]pyridin-7-yl)piperazine-1-carboxylate (1.4 g, 3.6 mmol) in DMF/H₂O (10 mL/2 mL) was added K₂CO₃ (1.49 g, 10.8 mmol), followed by 2-iodoacetonitrile (482.4 mg, 7.2 mmol). The reaction mixture was stirred for overnight, poured into H₂O (30 mL), and extracted with EA (30 mL×3). The combined organic layers were washed with brine, dried over Na₂SO₄ and concentrated to dryness. The resulting residue was purified by flash column chromatography to give the titled compound (1 g, 65%).MS: M/e 429 (M+1)⁺.

### Step C: 2-(7-((2S,5R)-2,5-diethylpiperazin-1-yl)-4-methyl-5-oxo-4.5-dihydro-2H-pyrazolo[4,3-b]pyridin-2-yl)acetonitrile (Intermediate 3)

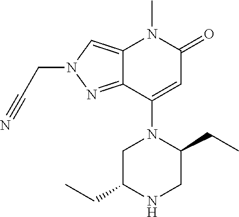 [SMILES: CC[C@@H]1CN(c2cc(=O)n(C)c3cn(CC#N)nc23)[C@@H](CC)CN1]

To a stirred solution of tert-butyl (2R,5S)-4-(2-(cyanomethyl)-4-methyl-5-oxo-4,5-dihydro-2H-pyrazolo[4,3-b]pyridin-7-yl)-2,5-diethylpiperazine-1-carboxylate (1 g, 2.34 mmol) in CH₂Cl₂ (15 mL) was added TFA (3 mL). Then reaction mixture was stirred for 5 hours, concentrated to give a residue, basified to pH=10-11 with saturated NaHCO₃ aq., and extracted with CH₂Cl₂/ IPA (3/1, 30 mL×3). The combined organic layers were washed with brine, dried over Na₂SO₄, and concentrated to give the titled compound (752 mg, 98%). ¹H NMR (400 MHz, DMSO-d6) δ 7.94 (s, 1H), 5.59 (s, 2H), 5.35 (s, 1H), 4.31 (s, 1H), 4.08 (d, J=13.2 Hz, 1H), 3.23 (s, 3H), 3.04 (dd, J=12.8, 4.4 Hz, 1H), 2.80-2.72 (m, 1H), 2.65-2,59 (m, 1H), 1.83-1.72 (m, 1H), 1.64-1.41 (m, 3H), 0.87-0.75 (m, 8H) ppm. MS: We 329 (M+1)⁺.

### Intermediate 4: 4-methyl-2-(tetrahydro-2H-pyran-2-yl)-2,4-dihydro-5H-pyrazolo[4,3-d]pyrimidine-5,7 (6H)-dione

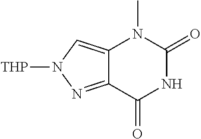

### Step A: 4-nitro-1-(tetrahydro-2H-pyran-2-yl)-1H-pyrazole-3-carboxamide

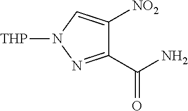

To ethyl-4-nitro-1-(tetrahydro-2H-pyran-2-yl)-1H-pyrazole-3-carboxylate (8 g, 29.7 mmol) was added a solution of NH₃ (7M) in MeOH (80 mL). The reaction mixture was stirred at 80° C. for 16 hours, cooled to room temperature and concentrated to dryness. The resulting residue was treated to slurry with PE/EA to give the titled compound (6.8 g, 95%). MS: M/e 241 (M+1)⁺.

### Step B: 4-amino-1-(tetrahydro-2H-pyran-2-yl)-1H-pyrazole-3-carboxamide

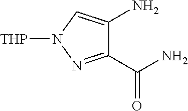

To a solution of 4-nitro-1-(tetrahydro-2H-pyran-2-yl)-1H-pyrazole-3-carboxamide (6.2 g, 25.8 mmol) in MeOH (100 mL) was added Pd/C (0.6 g, 10% in water). The resulting mixture was stirred at room temperature under H₂ atmosphere. The reaction mixture was filtered and washed with MeOH. The combined filtrate was concentrated to give the titled compound (5.1 g, 94%). MS: M/e 211 (M+1)⁺.

### Step C: 4-((2,4-dinitrophenyl)sulfonamido)-1-(tetrahydro-2H-pyran-2-yl)-1H-pyrazole-3-carboxamide

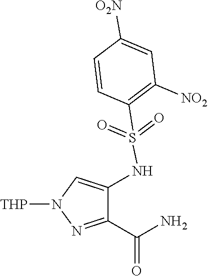

To a solution of 4-amino-1-(tetrahydro-2H-pyran-2-yl)-1H-pyrazole-3-carboxamide (4.6 g, 21.9 mmol) and TEA (3.33 g, 33 mmol) in THF (0.2 L) was added 2,4-dinitrobenzenesulfonyl chloride (6.11 g 23 mmol) dropwise. The reaction mixture was stirred at room temperature for 6 hours and concentrated to dryness. The resulting residue was diluted with water and extracted with DCM (100 mL×3). The combined organic layers were washed with brine, dried over Na₂SO₄, filtered and concentrated to dryness. The resulting residue was purified by column flash column chromatography to give the titled compound (5.3 g, 55%). MS: M/e 441 (M+1)⁺.

### Step D: 4-((N-methyl-2,4-dinitrophenyl)sulfonamido)-1-(tetrahydro-2H-pyran-2-yl)-1H-pyrazole-3-carboxamide

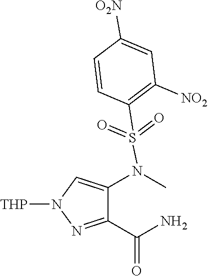

To a solution of 4-((2,4-dinitrophenyl)sulfonamido)-1-(tetrahydro-2H-pyran-2-yl)-1H-pyrazole-3-carboxamide (5.3 g, 12 mmol) in DMF (60 mL) was added K₂CO₃ (3.3 g, 24 mmol), followed by CH₃I (3.4 g, 24 mmol). The reaction mixture was stirred at RT for 16 hours. The reaction solvent was concentrated to dryness. The resulting residue was diluted with water and extracted with DCM (100 mL×2). The combined organic layers were washed with brine, dried over Na₂SO₄, filtered, and concentrated to dryness. The resulting residue was purified by flash column chromatography to give the titled compound (4.8 g, 88%). MS: M/e 455 (M+1)⁺.

### Step E: 4-(methylamino)-1-(tetrahydro-2H-pyran-2-v-1H-pyrazole-3-carboxamide

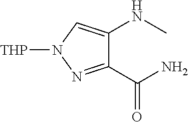

To a solution of 4-((N-methyl-2,4-dinitrophenyl)sulfonamido)-1-(tetrahydro-2H-pyran-2-yl)-1H-pyrazole-3-carboxamide (4.8 g, 10.57 mmol) and TEA (2.13 g, 21.14 mmol) in DCM (40 mL) was added 2-mercaptoacetic acid (1.36 g, 14.8 mmol). The reaction mixture was stirred for 6 hours. The reaction was quenched with water. The resulting mixture was extracted with DCM:IPA (3:1, 100 mL×5). The combined organic layers were washed with brine, dried over Na₂SO₄, filtered and concentrated to dryness. The resulting residue was purified by flash column chromatography (DCM:MeOH=10:1) to give the titled compound (1.8 g, 75%). MS: M/e 225 (M+1)⁺.

### Step F: 4-methyl-2-(tetrahydro-2H-pyran-2-yl)-2,4-dihydro-5H-pyrazolo[4,3-d]pyrimidine-5.7 (6H)-dione (Intermediate 4)

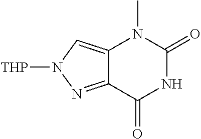

To a solution of 4-(methylamino)-1-(tetrahydro-2H-pyran-2-yl)-1H-pyrazole-3-carboxamide (680 mg, 3 mmol) in DMF (15 mL) was added NaH (240 mg, 6 mmol) at 0° C. After 1 hour, CDI (972 mg, 6 mmol) was added to the reaction and the resulting mixture was heated to 80° C. for overnight. The reaction mixture was cooled to room temperature, quenched with H₂O and concentrated to dryness. The resulting residue was purified by flash column chromatography (DCM:MeOH=10:1) to give the titled compound (0.6 g, 80%). ¹H NMR (400 MHz, DMSO-d₆) δ 11.19 (s, 1H), 8.15 (s, 1H), 5.55 (dd, J=2,4, 9.2 Hz, 1H), 3.98-3.89 (m, 1H), 3.74-3.63 (m, 1H), 3.27 (s, 3H), 2.15-1.85 (m, 3H), 1.78-1.63 (m, 1H), 1.62-1.50 (m, 2H) ppm. MS: M/e 251 (M+1)⁺.

### Intermediate 5: 2-(7-((2S,5R)-2,5-dimethylpiperazin-1-yl)-4-methyl-5-oxo-4,5-dihydro-2H-pyrazolo[4,3-b]pyridin-2-yl)acetonitrile

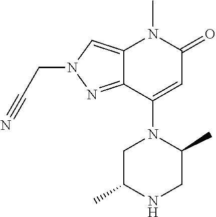 [SMILES: C[C@@H]1CN(c2cc(=O)n(C)c3cn(CC#N)nc23)[C@@H](C)CN1]

### Step A: tert-butyl (2R,5S)-2,5-dimethyl-4-(4-methyl-5-oxo-2-(tetrahydro-2H-pyran-2-yl)-4,5-dihydro-2H-pyrazolo[4,3-b]pyridin-7-yl)piperazine-1-carboxylate (Intermediate 5A)

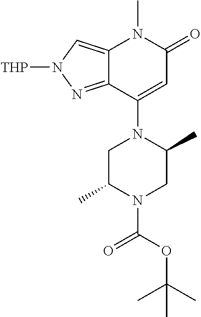

To a solution of tert-butyl (2R,5S)-2,5-dimethylpiperazine-1-carboxylate (2,56 g, 12 mmol) and Intermediate 2 (3.81 mg, 10 mmol) in CH₃CN (15 mL) was added DIPEA (3.87 g, 30 mmol). Then the mixture was heated at 90° C. under N₂ for 16 hours. The mixture was cooled to room temperature, diluted with water (150 mL), extracted with EA (80 mL×3). The combined organic layers were washed with brine, dried over Na₂SO₄, filtered and concentrated to dryness. The resulting residue was The resulting residue was purified by flash column chromatography (DCM:MeOH=15:1) to give titled compound (4.9 g, crude). MS: M/e 446 (M+1)⁺.

### Step B: 7-((2S,5R)-2,5-dimethylpiperazin-1-yl)-4-methyl-2-(tetrahydro-2H-pyran-2-yl)-2,4-dihydro-5H-pyrazolo[4,3-b]pyridin-5-one (Intermediate 5B)

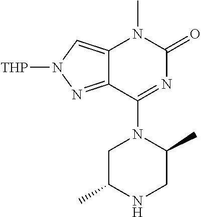

To a solution of Intermediate 5A (4.9 g, crude) in DCM (20 mL) was added TFA (5 mL). The resulting mixture was stirred at room temperature overnight. The mixture was diluted with water, basified with sat NaHCO₃ solution, extracted with DCM:IPA (3:1, 80 mL×3), dried over Na₂SO₄, filtered, and concentrated to dryness. The resulting residue was purified by flash column chromatography (DCM:MeOH=5:1, 1 mol/L NH₃ in MeOH) to give titled compound (2.9 g, 84% for 2 steps). 1H NMR (400 MHz, CD₃OD) S 8.03 (d, J=2.8 Hz, 1H), 5.65 (s, 1H), 5.60-5.32 (m, 1H), 4.85-4.76 (m, 1H), 4.53-4.42 (m, 1H), 4.11-3.96 (m, 1H), 3.82-3.70 (m, 1H), 3.69-3.57 (m, 2H), 3.56-3.49 (m, 1H), 3.46 (s, 3H), 3.04-2.92 (m, 1H), 2.30-2.00 (m, 3H), 1.85-1.60 (m, 3H), 1.42-1.30 (m, 6H) ppm. MS: M/e 346 (M+1)⁺.

### Step C: tert-butyl (2R,5S)-2,5-dimethyl-4-(4-methyl-5-oxo-4,5-dihydro-2H-pyrazolo[4,3-b]pyridin-7-yl)piperazine-1-carboxylate

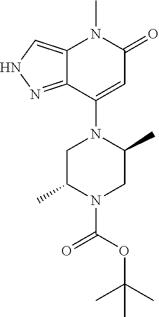 [SMILES: C[C@@H]1CN(c2cc(=O)n(C)c3c[nH]nc23)[C@@H](C)CN1C(=O)OC(C)(C)C]

To a stirred solution of Intermediate 5B (3 g, 8.70 mmol) in MeOH (20 mL) was added dioxane/HCl(g) (4.0 M, 5 mL). After the addition, the reaction was stirred over a weekend. The reaction mixture was concentrated to give The resulting residue, which was treated with THF/H₂O (50 mL/20 mL), K₂CO₃ (3.60 g, 26.08 mmol) was added, followed by Boc₂O(1.90 g, 8.70 mmol). Then the mixture was stirred for 1 hour. The mixture was acidified to pH=4-5 with citric acid, extracted with EtOAc (30 mL×3). The combined organic layers were washed with brine, dried over Na₂SO₄, concentrated to dryness. The resulting residue was purified by flash column chromatography to give the titled compound (1.7 g, 54%). MS: M/e 362 (M+1)⁺.

### Step D: tert-butyl (2R,5S)-4-(2-(cyanomethyl)-4-methyl-5-oxo-45-dihydro-2H-pyrazolo[4,3-b]pyridin-7-yl)-2,5-dimethylpiperazine-1-carboxylate

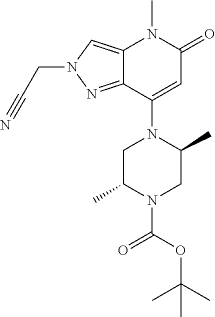 [SMILES: C[C@@H]1CN(c2cc(=O)n(C)c3cn(CC#N)nc23)[C@@H](C)CN1C(=O)OC(C)(C)C]

To a stirred solution of tert-butyl (2R,5S)-2,5-dimethyl-4-(4-methyl-5-oxo-4,5-dihydro-2H-pyrazolo[4,3-b]pyridin-7-yl)piperazine-1-carboxylate (1.7 g, 4.71 mmol) in DMF/H₂O (10 mL/2 mL) was added K₂CO₃ (1.95 g, 14.1 mmol), followed by 2-iodoacetonitrile (1.18 g, 7.06 mmol). After the addition, the reaction mixture was stirred at RT overnight. The reaction mixture was poured into H₂O (30 mL), extracted with EtOAc (30 mL×3). The combined organic layers were washed with brine, dried over Na₂SO₄, concentrated to dryness. The resulting residue was purified by flash column chromatography to give the titled compound (1.3 g, 69%). MS: M/e 401 (M+1)⁺.

### Step E: 2474 (2S,5R)-2,5-dimethylpiperazin-1-yl)-4-methyl-5-oxo-4.5-dihydro-2H-pyrazolo[4,3-b]pyridin-2-yl)acetonitrile

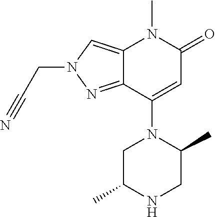 [SMILES: C[C@@H]1CN(c2cc(=O)n(C)c3cn(CC#N)nc23)[C@@H](C)CN1]

To a stirred solution of tert-butyl (2R,5S)-4-(2-(cyanomethyl)-4-methyl-5-oxo-4,5-dihydro-2H-pyrazolo[4,3-b]pyridin-7-yl)-2,5-dimethylpiperazine-1-carboxylate (1.3 g, 3.25 mmol) in CH₂Cl₂ (15 mL) was added TFA (3 mL). Then the mixture was stirred for 5 hours. The reaction mixture was concentrated to give The resulting residue, basified to pH=10-11 with aq. NaHCO₃, extracted with CH₂Cl₂/IPA (3/1, 30 mL×3). The combined organic layers were washed with brine, dried over Na₂SO₄ and concentrated to give the titled compound (870 mg, 89%). MS: M/e 301 (M+1)⁺.

### Intermediate 6: 2-(but-2-yn-1-yl)-7-((2S,5R)-5-ethyl-2-methylpiperazin-1-yl)-4-methyl-2,4-dihydro-5H-pyrazolo[4,3-b]pyridin-5-one

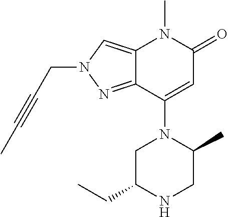 [SMILES: CC#CCn1cc2c(n1)c(N1C[C@@H](CC)NC[C@@H]1C)cc(=O)n2C]

### Step A: tert-butyl(2R,5S)-2-ethyl-5-methyl-4-(4-methyl-5-oxo-24tetrahydro-2H-pyran-2-yl)-4.5-dihydro-2H-pyrazolo[4,3-b]pyridin-7-yl)piperazine-1-carboxylate

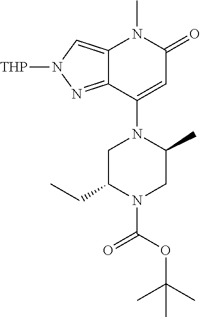

A mixture of 4-methyl-5-oxo-2-(tetrahydro-2H-pyran-2-yl)-4,5-dihydro-2H-pyrazolo[4,3-b]pyridin-7-yl trifluoromethanesulfonate (19 g, 50 mmol), tert-butyl (2R,5S)-2-ethyl-5-methylpiperazine-1-carboxylate (12,54 g, 55 mmol) and DIPEA(12.9 g, 0.1 mol) in CH₃CN (200 mL) was stirred at 100° C. overnight in a sealed tube. The reaction mixture was concentrated to dryness. The resulting residue was dissolved in CH₂Cl₂ (200 mL) and washed with H₂O, brine, dried over Na₂SO₄ and concentrated to dryness. The resulting residue was purified by flash column chromatography to give the titled compound (20 g, 83.5%). MS: We 480 (M+1)⁺.

### Step B:7-((2S,5R)-5-ethyl-2-methylpiperazin-1-yl)-4-methyl-2,4-dihydro-5H-pyrazolo[4,3-b]pyridin-5-one

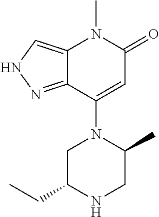 [SMILES: CC[C@@H]1CN(c2cc(=O)n(C)c3c[nH]nc23)[C@@H](C)CN1]

To a stirred solution of tert-butyl (2R,5S)-2-ethyl-5-methyl-4-(4-methyl-5-oxo-2-(tetrahydro-2H-pyran-2-yl)-4,5-dihydro-2H-pyrazolo[4,3-b]pyridin-7-yl)piperazine-1-carboxylate (6 g, 12,5 mmol) in MeOH (50 mL) was added dioxane/HCl(g) (4.0 M, 50 mL). Then the mixture was stirred for 3 days at 50° C. The reaction mixture was concentrated to give the titled compound (crude), which was used to the next step directly. MS: M/e 276 (M+1)⁺.

### Step C: tert-butyl (2R,5S)-2-ethyl-5-methyl-4-(4-methyl-5-oxo-4,5-dihydro-2H-pyrazolo[4,3-b]pyridin-7-yl)piperazine-1-carboxylate

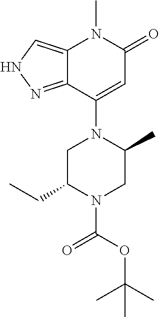 [SMILES: CC[C@@H]1CN(c2cc(=O)n(C)c3c[nH]nc23)[C@@H](C)CN1C(=O)OC(C)(C)C]

7-((2S,5R)-5-ethyl-2-methylpiperazin-1-yl)-4-methyl-2,4-dihydro-5H-pyrazolo[4,3-b]pyridin-5-one (12,5 mmol) was dissolved in THF/H₂O (80 mL/80 mL), then basified to pH=7-8 with NaHCO₃. To the reaction mixture was added NaHCO₃ (2.1 g, 25 mmol), followed by Boc₂₀ (3.28 g, 15 mmol). After the addition, the reaction mixture was stirred for 4 hours. The reaction mixture was extracted with EtOAc (50 mL×2). The combined organic layers were washed with brine, dried over Na₂SO₄ and concentrated to dryness. The resulting residue purified by flash column chromatography to give the titled compound (3.8 g, 81%). MS: M/e 376 (M+1)⁺.

### Step D: tert-butyl (2R,5S)-4-(2-(but-2-yn-1-yl-4-methyl-5-oxo-4,5-dihydro-2H-pyrazolo[4,3-b]pyridin-7-yl)-2-ethyl-5-methylpiperazine-1-carboxylate

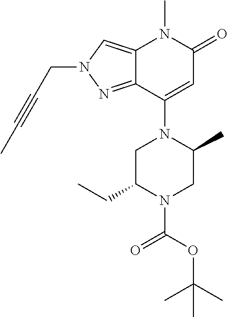 [SMILES: CC#CCn1cc2c(n1)c(N1C[C@@H](CC)N(C(=O)OC(C)(C)C)C[C@@H]1C)cc(=O)n2C]

To a stirred solution of tert-butyl (2R,5S)-2-ethyl-5-methyl-4-(4-methyl-5-oxo-4,5-dihydro-2H-pyrazolo[4,3-b]pyridin-7-yl)piperazine-1-carboxylate (3.8 g, 10.1 mmol) in DMF/H₂O (30 mL/10 mL) was added K₂CO₃ (2.78 g, 20.2 mmol), followed by 1-bromobut-2-yne (2.02 g, 15.2 mmol). After the addition, the reaction was stirred at RT overnight. The reaction mixture was poured into H₂O (50 mL), then extracted with EtOAc (40 mL×3). The combined organic layers were washed with brine, dried over Na₂SO₄, concentrated to dryness. The resulting residue was purified by flash column chromatography to give the titled compound (2.2 g, 51%). MS: M/e 428 (M+1)⁺.

### Step E: 2-(but-2-yn-1-yl)₇-((2S,5R_{1˜5}-ethyl-2-methylpiperazin-1-yl)-4-methyl-2,4-dihydro-5H-pyrazolo[4,3-b]pyridin-5-one

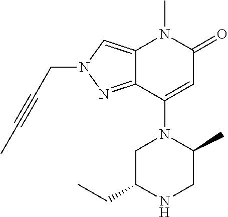 [SMILES: CC#CCn1cc2c(n1)c(N1C[C@@H](CC)NC[C@@H]1C)cc(=O)n2C]

To a stirred solution of tert-butyl (2R,5S)-4-(2-(but-2-yn-1-yl)-4-methyl-5-oxo-4,5-dihydro-2H-pyrazolo[4,3-b]pyridin-7-yl)-2-ethyl-5-methylpiperazine-1-carboxylate (2.2 g, 5.15 mmol) in CH₂Cl₂ (50 mL) was added TMSOTf (2.3 g, 10.3 mmol). After then, the mixture was stirred for 2 hours. The mixture was quenched with aq. Na₂CO₃, extracted with CH₂Cl₂/IPA (3/1, 50 mL×4). The combined organic layers were washed with brine, dried over Na₂SO₄ and concentrated to give the titled compound (1.8 g, 99%). MS: M/e 328 (M+1)⁺.

### Intermediate 7: 2-(but-2-yn-1-yl)-7-((2S,5R)-2,5-diethylpiperazin-1-yl)-4-methyl-2,4-dihydro-5H-pyrazolo[4,3-b]pyridin-5-one

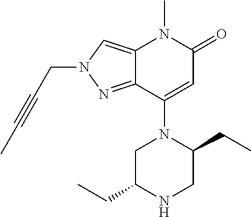 [SMILES: CC#CCn1cc2c(n1)c(N1C[C@@H](CC)NC[C@@H]1CC)cc(=O)n2C]

### Step A: tert-butyl (2R,5S)-2,5-diethyl-4-(4-methyl-5-oxo-2-(tetrahydro-2H-pyran-2-yl)-4,5-dihydro-2H-pyrazolo 4-b]pyridin-7-yl)piperazine-2-carboxylate

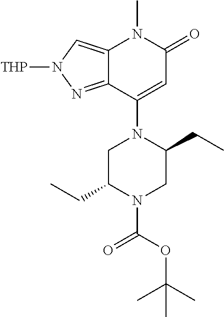

A mixture 4-methyl-5-oxo-2-(tetrahydro-2H-pyran-2-yl)-4,5-dihydro-2H-pyrazolo[4,3-b]pyridin-7-yl trifluoromethanesulfonate (5 g, 13.1 mmol), t tert-butyl (2R,5S)-2,5-diethylpiperazine-1-carboxylate (3.8 g, 15.7 mmol) and DIPEA (4.2 g, 32.8 mmol) in DMAc (50 mL) was stirred at 100° C. overnight. The reaction mixture was diluted with CH₂Cl₂ (300 mL) and extracted with H₂O (500 mL×3). The combined organic layers were washed with brine, dried over Na₂SO₄ and concentrated to dryness. The resulting residue was purified by flash column chromatography to give the titled compound (7.5 g).MS: M/e 474 (M+1)⁺.

### Step B: 7-((2S,5R-2,5-diethylpiperazin-1-yl)-4-methyl-2,4-dihydro-5H-pyrazolo[4,3-b]pyridin-5-one

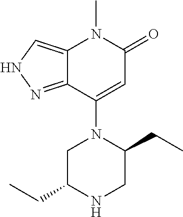 [SMILES: CC[C@@H]1CN(c2cc(=O)n(C)c3c[nH]nc23)[C@@H](CC)CN1]

To a stirred solution of tert-butyl (2R,5S)-2,5-diethyl-4-(4-methyl-5-oxo-2-(tetrahydro-2H-pyran-2-yl)-4,5-dihydro-2H-pyrazolo[4,3-b]pyridin-7-yl)piperazine-1-carboxylate (7.5 g, 15.8 mmol) in MeOH (50 mL) was added 1,4-dioxane/HCl(g) (4 M, 20 mL). After the addition, the reaction mixture was stirred at room temperature overnight. The reaction mixture was concentrated to give the titled compound (crude), which was used to the next step directly. MS: M/e 290 (M+1)⁺.

### Step C: tert-butyl (2R,5S)_{2,5}-diethyl-4-(4-methyl-5-oxo-4,5-dihydro-2H-pyrazolo[4,3-b]pyridin-7-yl)piperazine-1-carboxylate

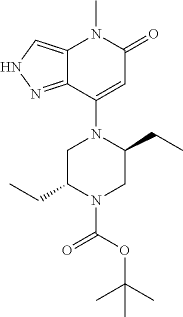 [SMILES: CC[C@@H]1CN(c2cc(=O)n(C)c3c[nH]nc23)[C@@H](CC)CN1C(=O)OC(C)(C)C]

To a stirred solution of 7-((2S,5R)-2,5-diethylpiperazin-1-yl)-4-methyl-2,4-dihydro-5H-pyrazolo[4,3-b]pyridin-5-one (crude, 15.8 mmol) in THF (50 mL)/H₂O (20 mL) was added NaHCO₃ (4 g, 47.4 mmol), followed by (Boc)₂₀ (6.8 g, 31.6 mmol). The reaction mixture was stirred at room temperature overnight. The reaction mixture was extracted with CH₂Cl₂ (200 mL×2), dried over Na₂SO₄ and concentrated to dryness. The resulting residue was purified by flash column chromatography to give the titled compound (4.4 g, three steps 86%).MS: M/e 390 (M+1)⁺.

### Step D: tert-butyl (2R,5S)-4-(2-(but-2-yn-1-yl)-4-methyl-5-oxo-4,5-dihydro-2H-pyrazolo[4,3-b]pyridin-7-yl)-2,5-diethylpiperazine-1-carboxylate

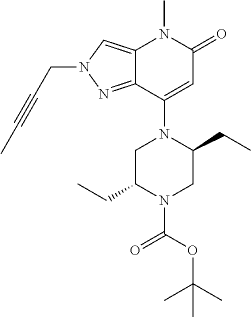 [SMILES: CC#CCn1cc2c(n1)c(N1C[C@@H](CC)N(C(=O)OC(C)(C)C)C[C@@H]1CC)cc(=O)n2C]

To a stirred solution of tert-butyl (2R,5S)-2,5-diethyl-4-(4-methyl-5-oxo-4,5-dihydro-2H-pyrazolo[4,3-b]pyridin-7-yl)piperazine-1-carboxylate (4.4 g, 11.3 mmol) in DMF (50 mL)/H₂O (10 mL) was added K₂CO₃ (4.7 g, 33.9 mmol), followed by 1-bromobut-2-yne (3 g, 22.6 mmol). The reaction mixture was stirred at room temperature overnight. The reaction mixture was extracted with CH₂Cl₂ (200 mL×2), washed with brine, dried over Na₂SO₄ and concentrated to dryness. The resulting residue was purified by flash column chromatography to give the titled compound (2,5 g, 50%).MS: M/e 442 (M+1)⁺.

### Step E: 2-(but-2-yn-1-yl)-74 (2S,5R)-2,5-diethylpiperazin-1-yl)-4-methyl-2,4-dihydro-5H-pyrazolo[4,3-b]pyridin-5-one

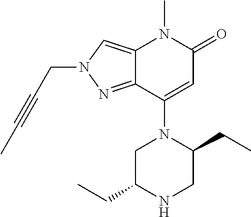 [SMILES: CC#CCn1cc2c(n1)c(N1C[C@@H](CC)NC[C@@H]1CC)cc(=O)n2C]

To a stirred solution of tert-butyl (2R,5S)-4-(2-(but-2-yn-1-yl)-4-methyl-5-oxo-4,5-dihydro-2H-pyrazolo[4,3-b]pyridin-7-yl)-2,5-diethylpiperazine-1-carboxylate (2,5 g, 5.7 mmol) in CH₂Cl₂ (30 mL) was added trimethylsilyl trifluoromethanesulfonate (1.9 g, 8.5 mmol). The reaction mixture was stirred at room temperature overnight. The mixture was quenched with aq. Na₂CO₃, extracted with CH₂Cl₂/IPA (3/1, 100 mL×3). The combined organic layers were washed with brine, dried over Na₂SO₄ and concentrated to dryness. The resulting residue was purified by flash column chromatography to give the titled compound (2 g, 100%). MS: M/e 342 (M+1)⁺.

### Intermediate 8: 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile

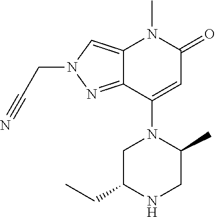 [SMILES: CC[C@@H]1CN(c2cc(=O)n(C)c3cn(CC#N)nc23)[C@@H](C)CN1]

### Step A: tert-butyl (2R,5S)-4-(2-(cyanomethyl)-4-methyl-5-oxo-4,5-dihydro-2H-pyrazolo[4,3-b]pyridin-7-yl)-2-ethyl-5-methylpiperazine-1-carboxylate

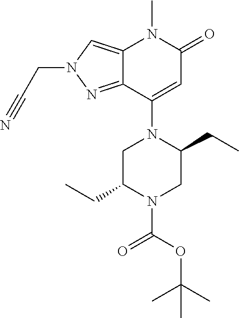 [SMILES: CC[C@@H]1CN(c2cc(=O)n(C)c3cn(CC#N)nc23)[C@@H](CC)CN1C(=O)OC(C)(C)C]

To a stirred solution of tert-butyl (2R,5S)-2-ethyl-5-methyl-4-(4-methyl-5-oxo-4,5-dihydro-2H-pyrazolo[4,3-b]pyridin-7-yl)piperazine-1-carboxylate (14 g, 37.3 mmol) in DMF (100 mL) was added K₂CO₃ (10.3 g, 74.7 mmol), followed by 2-chloroacetonitrile (4.2 g, 56 mmol). After the addition, the reaction was stirred overnight. The reaction mixture was poured into H₂O (300 mL), then extracted with EtOAc (150 mL×3). The combined organic layers were washed with brine, dried over Na₂SO₄, concentrated and purified by flash column chromatography to give the titled compound (14 g, 90.7%). MS: M/e 415 (M+1)⁺.

### Step B: 2-(74 (2S,5R)-5-ethyl-2-methylpiperazin-1-yl-4-methyl-5-oxo-4,5-dihydro-2H-pyrazolo[4,3-b]pyridin-2-yl)acetonitrile

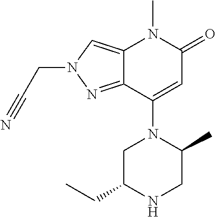 [SMILES: CC[C@@H]1CN(c2cc(=O)n(C)c3cn(CC#N)nc23)[C@@H](C)CN1]

To a stirred solution of tert-butyl (2R,5S)-4-(2-(cyanomethyl)-4-methyl-5-oxo-4,5-dihydro-2H-pyrazolo[4,3-b]pyridin-7-yl)-2-ethyl-5-methylpiperazine-1-carboxylate (14 g, 33.8 mmol) in CH₂Cl₂ (200 mL) was added TMSOTf (15 g, 67.6 mmol). After then, the mixture was stirred for 2 hours. The mixture was quenched with aq. Na₂CO₃, extracted with CH₂Cl₂/IPA (3/1, 50 mL×4). The combined organic layers were washed with brine, dried over Na₂SO₄, concentrated to give the target compound (10 g, 94.2%). MS: M/e 315 (M+1)⁺.

### Compound A1: 2-(7-((2S,5R)-2,5-diethyl-4-(1-(4-(3-methoxyazetidine-1-carbonyl)phenyl)ethyl)piperazin-1-yl-4-methyl-5-oxo-4,5-dihydro-2H-pyrazolo[4,3-b]pyridin-2-yl)acetonitrile

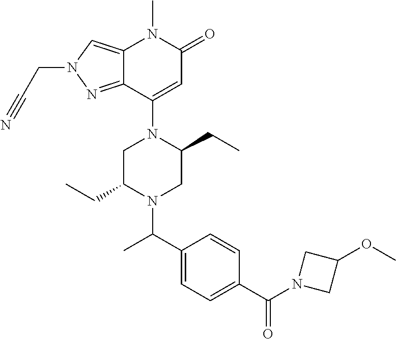 [SMILES: CC[C@H]1CN(C(C)c2ccc(C(=O)N3CC(OC)C3)cc2)[C@H](CC)CN1c1cc(=O)n(C)c2cn(CC#N)nc12]

### Step A: 1-(4-(3-methoxyazetidine-1-carbonyl)phenyl)ethan-1-one

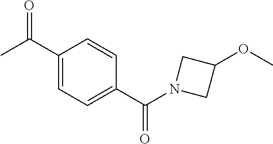 [SMILES: COC1CN(C(=O)c2ccc(C(C)=O)cc2)C1]

To a solution of 4-acetylbenzoic acid (164 mg, 1 mmol), 3-methoxyazetidine hydrochloride (150 mg, 1,2 mmol) and HATU (456 mg, 1,2 mmol) in DCM (10 mL) was added DIPEA (258 mg, 2 mmol). The reaction mixture was stirred at 25° C. overnight. The mixture was diluted with water, extracted with DCM (80 mL), washed with brine, dried over Na₂SO₄, filtered, and concentrated to dryness (300 mg, crude). MS: M/e 234 (M+1)⁺.

### Step B: (4-(1-hydroxyethyl)phenyl)(3-methoxyazetidin-1-yl)methanone

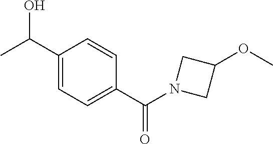 [SMILES: COC1CN(C(=O)c2ccc(C(C)O)cc2)C1]

To a solution of 1-(4-(3-methoxyazetidine-1-carbonyl)phenyl)ethan-1-one (300 mg, crude) in MeOH (5 mL) was added NaBH₄ (76 mg, 2 mmol) slowly at 0° C. The resulting mixture was stirred at room temperature for 2 hours. The reaction mixture was quenched with H₂O (0.5 mL) and concentrated to dryness. The resulting residue was purified by flash column chromatography to give the desired product (160 mg, 68% for two steps). ¹H NMR (400 MHz, DMSO-d₆) δ 7.58 (d, J=8.0 Hz, 2H), 7.41 (d, J=8.0 Hz, 2H), 5.26 (d, J=4.4 Hz, 1H), 4.80-4.69 (m, 1H), 4.47-4.36 (m, 1H), 4.28-4.06 (m, 3H), 3.89-3.75 (m, 1H), 3.21 (s, 3H), 1.32 (d, J=6.4 Hz, 3H) ppm.

### Step C: 2-(7-((2S,5R)-2,5-diethyl-4-(1-(4-(3-methoxyazetidine-1-carbonyl)phenyl)ethyl)piperazin-1-yl)-4-methyl-5-oxo-4,5-dihydro-2H-pyrazolo[4,3-b]pyridin-2-yl)acetonitrile

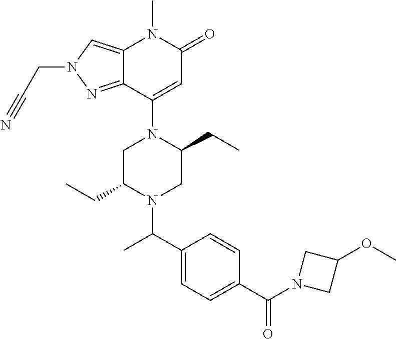 [SMILES: CC[C@H]1CN(C(C)c2ccc(C(=O)N3CC(OC)C3)cc2)[C@H](CC)CN1c1cc(=O)n(C)c2cn(CC#N)nc12]

To a solution of (4-(1-hydroxyethyl)phenyl)(3-methoxyazetidin-1-yl)methanone (46 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-HPLC (Method A) and Prep-HPLC (Method B) to give the titled Compound A1a (6 mg) and Compound A1b (10 mg).

Compound A1a (the earlier peak): ¹H NMR (500 MHz, CD₃OD) S 7.91 (s, 1H), 7.64 (d, J=8.0 Hz, 2H), 7.49 (d, J=8.0 Hz, 2H), 5.55 (s, 1H), 5.46 (s, 2H), 4.60-4.48 (m, 1H), 4.40-4.19 (m, 3H), 4.02-3.96 (m, 1H), 3.85-3.76 (m, 1H), 3.43 (s, 3H), 3.32 (s, 3H), 3.35-3.31 (m, 2H), 3.30-3.24 (m, 1H), 3.05-2.98 (m, 1H), 2.91-2.84 (m, 1H), 2,44-2.34 (m, 1H), 2.18-2.05 (m, 1H), 1.89-1.77 (m, 1H), 1.60-1.49 (m, 2H), 1.35 (d, J=7.5 Hz, 3H), 0.96 (t, J=7.5 Hz, 3H), 0.69 (t, J=7.5 Hz, 3H) ppm. MS: M/e 546 (M+1)

Compound A1b (the later peak): ¹H NMR (500 MHz, CD₃OD) S 7.92 (s, 1H), 7.61 (d, J=8.0 Hz, 2H), 7.51 (d, J=8.5 Hz, 2H), 5.55 (s, 1H), 5.46 (s, 2H), 4.57-4.44 (m, 1H), 4.40-4.14 (m, 3H), 4.02-3.94 (m, 1H), 3.72-3.63 (m, 1H), 3.53-3.47 (m, 1H), 3.43 (s, 3H), 3.37-3.31 (m, 5H), 3.19-3.12 (m, 1H), 2.71-2.61 (m, 1H), 2.36-2.28 (m, 1H), 2.02-1.89 (m, 1H), 1.73-1.50 (m, 3H), 1.32 (d, J=6.5 Hz, 3H), 1.02 (t, J=7.5 Hz, 3H), 0.62 (t, J=7.5 Hz, 3H) ppm. MS: M/e 546 (M+1)⁺.

### Compound A2: 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-N,N-dimethylbenzamide

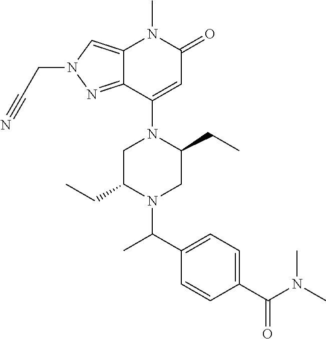 [SMILES: CC[C@H]1CN(C(C)c2ccc(C(=O)N(C)C)cc2)[C@H](CC)CN1c1cc(=O)n(C)c2cn(CC#N)nc12]

### Step A: 4-acetyl-N,N-dimethylbenzamide

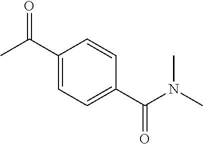

To a solution of 4-acetylbenzoic acid (164 mg, 1 mmol), dimethylamine hydrochloride (97 mg, 1,2 mmol) and HATU (456 mg, 1,2 mmol) in DCM (10 mL) was added DIPEA (258 mg, 2 mmol). The reaction mixture was stirred at 25° C. overnight. The mixture was diluted with water, extracted with DCM (80 mL), washed with brine, dried over Na₂SO₄, filtered, and concentrated to dryness (290 mg, crude). MS: M/e 192 (M+1)⁺.

### Step B: 4-(1-hydroxyethyl)-N,N-dimethylbenzamide

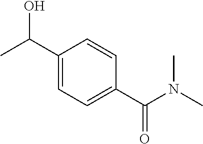

To a solution of 4-acetyl-N,N-dimethylbenzamide (290 mg, crude) in MeOH (5 mL) was added NaBH₄ (76 mg, 2 mmol) slowly at 0° C. The resulting mixture was stirred at room temperature for 2 hours. The reaction mixture was quenched with H₂O (0.5 mL) and concentrated to dryness. The resulting residue was purified by flash column chromatography to give the desired product (140 mg, 72% for two steps). ¹H NMR (400 MHz, DMSO-d₆) δ 7.39 (d, J=8.4 Hz, 2H), 7.36 (d, J=8.4 Hz, 2H), 5.22 (d, J=3.6 Hz, 1H), 4.80-4.68 (m, 1H), 2.97 (s, 3H), 2.91 (s, 3H), 1.32 (d, J=6.4 Hz, 3H) ppm.

### Step C: 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-N,N-dimethylbenzamide

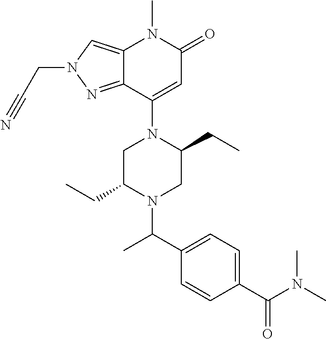 [SMILES: CC[C@H]1CN(C(C)c2ccc(C(=O)N(C)C)cc2)[C@H](CC)CN1c1cc(=O)n(C)c2cn(CC#N)nc12]

To a solution of 4-(1-hydroxyethyl)-N,N-dimethylbenzamide (39 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-HPLC (Method A) and Prep-HPLC (Method B) to give the titled Compound A2a (6 mg) and Compound A2b (9 mg).

Compound A2a (the earlier peak): ¹H NMR (500 MHz, CD₃OD) δ 7.91 (s, 1H), 7.48 (d, J=8.0 Hz, 2H), 7.42 (d, J=8.5 Hz, 2H), 5.55 (s, 1H), 5.46 (s, 2H), 3.83-3.76 (m, 1H), 3.43 (s, 3H), 3.35-3.31 (m, 2H), 3.28-3.24 (m, 1H), 3.11 (s, 3H), 3.02 (s, 3H), 3.02-2.98 (m, 1H), 2.91-2.84 (m, 1H), 2,46-2.35 (m, 1H), 2.16-2.05 (m, 1H), 1.87-1.76 (m, 1H), 1.59-1.48 (m, 2H), 1.36 (d, J=6.5 Hz, 3H), 0.96 (t, J=7.5 Hz, 3H), 0.69 (t, J=7.5 Hz, 3H) ppm. MS: M/e 504 (M+1)⁺.

Compound A2b (the later peak): ¹H NMR (500 MHz, CD₃OD) S 7.92 (s, 1H), 7.51 (d, J=8.0 Hz, 2H), 7.39 (d, J=8.0 Hz, 2H), 5.55 (s, 1H), 5.46 (s, 2H), 3.69-3.61 (m, 1H), 3.54-3.46 (m, 1H), 3.43 (s, 3H), 3.35-3.31 (m, 2H), 3.19-3.12 (m, 1H), 3.10 (s, 3H), 3.00 (s, 3H), 2.69-2.63 (m, 1H), 2.39-2.32 (m, 1H), 2.02-1.89 (m, 1H), 1.71-1.50 (m, 3H), 1.33 (d, J=7.0 Hz, 3H), 1.03 (t, J=7.5 Hz, 3H), 0.62 (t, J=7.5 Hz, 3H) ppm. MS: M/e 504 (M+1)⁺.

### Compound A3: 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-2,6-difluoro-N,N-dimethylbenzamide

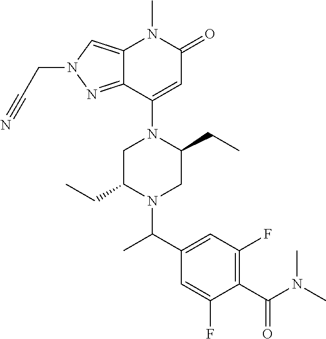 [SMILES: CC[C@H]1CN(C(C)c2cc(F)c(C(=O)N(C)C)c(F)c2)[C@H](CC)CN1c1cc(=O)n(C)c2cn(CC#N)nc12]

### Step A: 4-bromo-2,6-difluoro-N,N-dimethylbenzamide

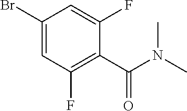

A solution of 4-bromo-2,6-difluorobenzoic acid (1 g, 4.22 mmol), dimethylamine hydrochloride (0.42 g, 5.12 mmol), HATU (2,41 g, 6.34 mmol) and DIPEA (1.63 g, 12.64 mmol) in DMF (10 ml) was stirred at RT overnight. The solution was poured into water (20 ml) and extracted with EA (15 ml×2). The organic layer was dried over Na₂SO₄ and concentrated to dryness. The resulting residue was purified by flash column chromatography with 0-30% EtOAc in PE to give the titled compound (1.1 g, 98%). MS: M/e 264 (M+1)⁺.

### Step B: 4-acetyl-2,6-difluoro-N,N-dimethylbenzamide

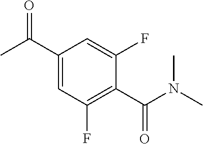 [SMILES: CC(=O)c1cc(F)c(C(=O)N(C)C)c(F)c1]

A solution of 4-bromo-2,6-difluoro-N,N-dimethylbenzamide (500 mg, 1.89 mmol), ethyl tributylstannanecarboxylate (850 mg, 2.35 mmol) and Pd(PPh₃)₂Cl₂ (66 mg, 0.094 mmol) in toluene(10 ml) was stirred at 100° C. overnight. The solution was cooled to RT. HCl/1,4-dioxane (4M, 2 ml) was added and stirred at RT for 10 min. The solution was diluted with EtOAc (20 ml), washed with aq. Na₂CO₃ (10 ml×2), dried over Na₂SO₄ and concentrated to dryness. The resulting residue was purified by flash column chromatography with 20-50% EA in PE to give the titled compound (430 mg, 100%). MS: M/e 228 (M+1)⁺.

### Step C: 2.6-difluoro-4-(1-hydroxyethyl)-N,N-dimethylbenzamide

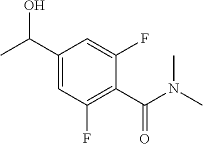 [SMILES: CC(O)c1cc(F)c(C(=O)N(C)C)c(F)c1]

A solution of 4-acetyl-2,6-difluoro-N,N-dimethylbenzamide (430 mg, 1.89 mmol) and NaBH₄ (72 mg, 1.89 mmol) in MeOH (6 ml) was stirred at RT for 15 min. The solution was diluted with EtOAc(15 ml), washed with brine (10 ml×2), dried over Na₂SO₄ and concentrated to dryness to give the titled compound (430 mg, 99%), which was used directly for the next step without further purification. MS: M/e 230 (M+1)⁺.

### Step D: 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-2.6-difluoro-N,N-dimethylbenzamide

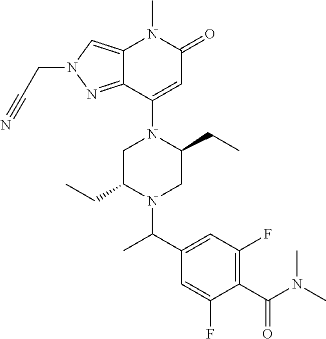 [SMILES: CC[C@H]1CN(C(C)c2cc(F)c(C(=O)N(C)C)c(F)c2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol), 2,6-difluoro-4-(1-hydroxyethyl)-N,N-dimethylbenzamide (52,4 mg, 0.23 mmol), (cyanomethyl)trimethylphosphonium iodide (111 mg, 0.46 mmol) and DIPEA (197 mg, 1.53 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A3, which was further separated into Compound A3a (4 mg) and Compound A3b (5 mg) by Prep-HPLC (Method A).

Compound A3a (the earlier peak): ¹H NMR (500 MHz, DMSO-d₆) δ 7.98 (s, 1H), 7.22 (d, J=9.2 Hz, 2H), 5.61 (s, 2H), 5.39 (s, 1H), 3.83 (q, J=6.5 Hz, 1H), 3.32-3.29 (m, 2H), 3.27 (s, 3H), 3.19 (d, J=11.2 Hz, 1H), 3.02 (s, 3H), 2.88 (s, 3H), 2.87-2.83 (m, 1H), 2.78 (dd, .=11.8, 3.5 Hz, 1H), 2.36 (s, 1H), 2.05-1.95 (m, 1H), 1.69-1.58 (m, 1H), 1.52-1.42 (m, 2H), 1,28 (d, J=6.5 Hz, 3H), 0.85 (t, J=7.3 Hz, 3H), 0.69 (t, J=7.3 Hz, 3H) ppm. MS: M/e 540 (M+1)⁺.

Compound A3b (the later peak): ¹H NMR (500 MHz, DMSO-d₆) δ 7.98 (s, 1H), 7.23 (t, J=10.1 Hz, 2H), 5.61 (s, 2H), 5.40 (s, 1H), 3.66 (q, J=6.2 Hz, 1H), 3.35 (d, J=22.3 Hz, 2H), 3.31-3.29 (m, 1H), 3.27 (s, 3H), 3.02 (s, 1H), 3.02 (s, 3H), 2.85 (s, 3H), 2,59 (d, J=9.9 Hz, 1H), 2.22 (d, J=11.8 Hz, 1H), 1.95-1.85 (m, 1H), 1.64-1.35 (m, 3H), 1,25 (d, J=6.5 Hz, 3H), 0.94 (t, .=7.3 Hz, 3H), 0.58 (t, J=7.3 Hz, 3H) ppm. MS: M/e 540 (M+1)⁺.

### Compound A4: 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-2-fluoro-N,N-dimethylbenzamide

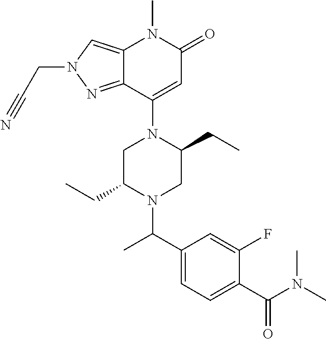 [SMILES: CC[C@H]1CN(C(C)c2ccc(C(=O)N(C)C)c(F)c2)[C@H](CC)CN1c1cc(=O)n(C)c2cn(CC#N)nc12]

### Step A: 4-acetyl-2-fluoro-N,N-dimethylbenzamide

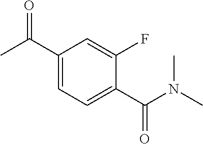

A mixture of 4-bromo-2-fluoro-N,N-dimethylbenzamide (500 mg, 2.04 mmol), tributyl(1-ethoxyvinyl)stannane (737 mg, 2.04 mmol) and Pd(PPh₃)₂Cl₂ (143 mg, 0.20 mmol) in toluene (10 mL) was stirred at 100° C. under N₂ overnight. The solution was added HCl (0.3 mL, 4 M in 1,4-dioxane) and the mixture was stirred at room temperature for 0.5 hour. The reaction mixture was diluted with EA and concentrated to dryness. The resulting residue was purified by flash column chromatography to give the titled compound (300 mg, 70%). MS: M/e 210 (M+1)⁺.

### Step B: 2-fluoro-4-(1-hydroxyethyl)-N,N-dimethylbenzamide

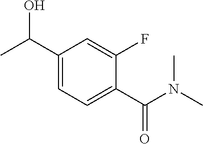

To a solution of 4-acetyl-2-fluoro-N,N-dimethylbenzamide (300 mg, 1.43 mmol) in MeOH (15 mL) was added NaBH₄ (109 mg, 2.87 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (220 mg, 73%). MS: M/e 212 (M+1)⁺.

### Step C: 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-2-fluoro-N,N-dimethylbenzamide

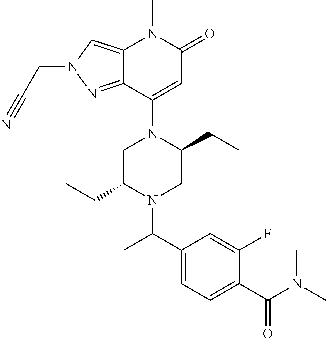 [SMILES: CC[C@H]1CN(C(C)c2ccc(C(=O)N(C)C)c(F)c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 2-fluoro-4-(1-hydroxyethyl)-N,N-dimethylbenzamide (32 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled Compound A4a (10 mg) and Compound A4b (12 mg, 28%).

Compound A4a (the earlier peak): ¹H NMR (400 MHz, CD₃OD) δ 7.90 (s, 1H), 7.34 (t, J=7.2 Hz, 1H), 7.29 (d, J=7.7 Hz, 1H), 7.23 (d, J=10.4 Hz, 1H), 5.55 (s, 1H), 5.44 (s, 2H), 4.81-4.11 (m, 2H), 3.81 (d, J=6.5 Hz, 1H), 3.46 (s, 1H), 3.41 (s, 3H), 3.10 (s, 3H), 2.99 (d, J=11.7 Hz, 1H), 2.95 (s, 3H), 2.86 (d, J=10.8 Hz, 1H), 2,42 (s, 1H), 2.16-2.05 (m, 1H), 1.87-1.77 (m, 1H), 1.58-1.48 (m, 2H), 1.33 (d, J=6.4 Hz, 3H), 0.94 (t, J=7.4 Hz, 3H), 0.71 (t, J=7.1 Hz, 3H). MS: M/e 522 (M+1)⁺.

Compound A4b (the later peak): ¹H NMR (400 MHz, CD₃OD) δ 7.90 (s, 1H), 7.33-7.26 (m, 3H), 5.54 (s, 1H), 5.45 (s, 2H), 4.78-4.00 (m, 2H), 3.66 (d, J=6.4 Hz, 1H), 3.49 (d, J=13.2 Hz, 1H), 3.41 (s, 3H), 3.14 (s, 1H), 3.10 (s, 3H), 2.93 (s, 3H), 2.67 (d, J=10.3 Hz, 1H), 2.33 (d, J=12.3 Hz, 1H), 2.01-1.92 (m, 1H), 1.71-1.61 (m, 2H), 1.56-1.49 (m, 1H), 1.31 (d, J=6.4 Hz, 3H), 1.00 (t, J=7.2 Hz, 3H), 0.64 (t, .=7.0 Hz, 3H). MS: M/e 522 (M+1)⁺.

### Compound A5: 2-(7-((2S,5R)-4-(1-(4-((1,1-dioxidothiomorpholino)methyl)-3,5-difluorophenyl)ethyl)-2,5-diethylpiperazin-1-yl)-4-methyl-5-oxo-4,5-dihydro-2H-pyrazolo[4,3-b]pyridin-2-yl)acetonitrile

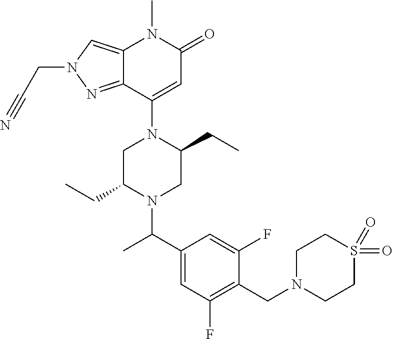 [SMILES: CC[C@H]1CN(C(C)c2cc(F)c(CN3CCS(=O)(=O)CC3)c(F)c2)[C@H](CC)CN1c1cc(=O)n(C)c2cn(CC#N)nc12]

### Step A: 5-bromo-2-(bromomethyl)-1,3-difluorobenzene

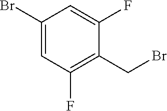 [SMILES: Fc1cc(Br)cc(F)c1CBr]

To a solution of 5-bromo-1,3-difluoro-2-methylbenzene (2.07 g, 10 mmol) and NBS (1.96 g, 11 mmol) in CH₃CN (20 mL) was added AIBN (50 mg). The reaction mixture was stirred at 95° C. under N₂ overnight. The mixture was cooled to room temperature, diluted with MTBE (40 mL), filtered, and the filtrate was concentrated to dryness. The resulting residue was purified by flash column chromatography to give the titled compound (2.63 g, 92%). ¹H NMR (500 MHz, CDCl₃) δ 7.15-7.10 (m, 2H), 4.64 (s, 2H) ppm.

### Step B: 4-(4-bromo-2,6-difluorobenzyl)thiomorpholine 1.1-dioxide

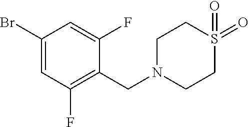 [SMILES: O=S1(=O)CCN(Cc2c(F)cc(Br)cc2F)CC1]

To a solution of 5-bromo-2-(bromomethyl)-1,3-difluorobenzene (855 mg, 3 mmol) and thiomorpholine 1,1-dioxide (405 mg, 3 mmol) in CH₃CN (6 mL) was added K₂CO₃ (822 mg, 6 mmol). The reaction was stirred at 25° C. overnight. The mixture was diluted with H₂O, extracted with EA (80 mL), washed with brine, dried over Na₂SO₄, filtered, and concentrated to dryness. The resulting residue was purified by flash column chromatography to give the titled compound (685 mg, 67%). MS: M/e 340 (M+1)⁺.

### Step C: 1-(4-((1.1-dioxidothiomorpholino)methyl)-3.5-difluorophenyl)ethan-1-one

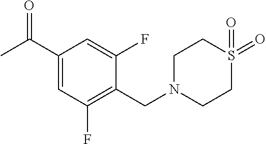 [SMILES: CC(=O)c1cc(F)c(CN2CCS(=O)(=O)CC2)c(F)c1]

To a solution of 4-(4-bromo-2,6-difluorobenzyl)thiomorpholine 1,1-dioxide (680 mg, 2 mmol) in toluene (8 mL) was added tributyl(1-ethoxyvinyl)stannane (948 mg, 3 mmol) and Pd(PPh₂)₂Cl₂ (70 mg, 0.1 mmol). The reaction mixture was protected by N₂ atmosphere and stirred at 100° C. overnight. The mixture was cooled down to RT, a solution of 4M HCl in dioxane (2 mL) was added. The resulting mixture was stirred at RT for 3 hours. The reaction was quenched with H₂O and saturated NaHCO₃ solution, extracted with EA (60 mL×2), washed with brine, dried over Na₂SO₄, filtered, and concentrated to dryness. The resulting residue was purified by flash column chromatography to give the titled compound (230 mg, 38%). MS: M/e 340 (M+1)⁺.

### Step D: 4-(2.6-difluoro-4-(1-hydroxyethyl)benzyl)thiomorpholine 1.1-dioxide

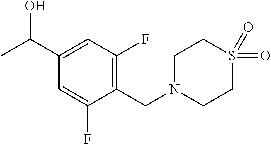 [SMILES: CC(O)c1cc(F)c(CN2CCS(=O)(=O)CC2)c(F)c1]

To a solution of 1-(4-((1,1-dioxidothiomorpholino)methyl)-3,5-difluorophenyl)ethan-1-one (230 mg, 0.76 mmol) in MeOH/DCM (8 mL/4 mL) was added NaBH₄ (32 mg, 0.85 mmol) slowly at 0° C. The resulting mixture was stirred at room temperature for 1 hour. The reaction mixture was quenched with H₂O (10 mL) and extracted with DCM (60 mL), washed with brine, dried over Na₂SO₄, filtered, and concentrated to dryness. The resulting residue was purified by flash column chromatography to give the desired product (160 mg, 69%). ¹H NMR (500 MHz, DMSO-d,) 6 7.11-7.04 (m, 2H), 5.39 (d, J=4.5 Hz, 1H), 4.76-4.69 (m, 1H), 3.74 (s, 2H), 3.12-3.06 (m, 4H), 2.90-2.83 (m, 4H), 1.31 (d, J=6.5 Hz, 3H) ppm.

### Step E: 2-(7-((2S,5R)-4-(1-(4-((1.1-dioxidothiomorpholino)methyl)-3.5-difluorophenyl)ethyl)-2,5-diethylpiperazin-1-yl-4-methyl-5-oxo-4,5-dihydro-2H-pyrazolo[4,3-b]pyridin-2-yl)acetonitrile

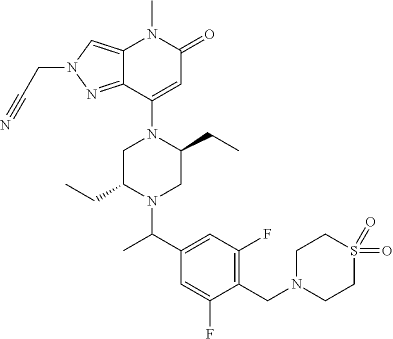 [SMILES: CC[C@H]1CN(C(C)c2cc(F)c(CN3CCS(=O)(=O)CC3)c(F)c2)[C@H](CC)CN1c1cc(=O)n(C)c2cn(CC#N)nc12]

To a solution of 4-(2,6-difluoro-4-(1-hydroxyethyl)benzyl)thiomorpholine 1,1-dioxide (60 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.6 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-TLC (DCM/MeOH=20/1) to give the titled compound (20 mg, 32%). ¹H NMR (500 MHz, CD₃OD) δ 7.94-7.91 (m, 1H), 7.12-7.02 (m, 2H), 5.58-5.54 (m, 1H), 5.49-5.44 (m, 2H), 3.83 (s, 2H), 3.81-3.77 (m, 1H), 3.68-3.58 (m, 0.5 H), 3.54-3.45 (m, 0.5H), 3.43 (s, 3H), 3.35-3.31 (m, 2H), 3.21-2.80 (m, 9.5H), 2.74-2.62 (m, 0.5H), 2,47-2.25 (m, 1H), 2.17-1.45 (m, 4H), 1.36-1,26 (m, 3H), 1.06-0.90 (m, 3H), 0.81-0.60 (m, 3H) ppm. MS: M/e 616 (M+1)⁺.

### Compound A6: 2-(7-((2S,5R)-4-(1-(2,4-dimethylphenyl)ethyl)-2,5-diethylpiperazin-1-yl)-4-methyl-5-oxo-4,5-dihydro-2H-pyrazolo[4,3-b]pyridin-2-yl)acetonitrile

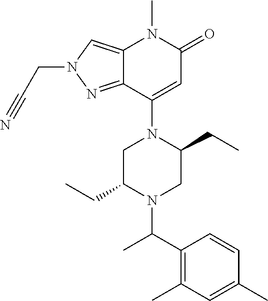 [SMILES: CC[C@H]1CN(C(C)c2ccc(C)cc2C)[C@H](CC)CN1c1cc(=O)n(C)c2cn(CC#N)nc12]

### Step A: 1-(2,4-dimethylphenyl)ethan-1-ol

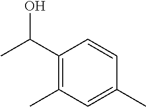

A solution of 1-(2,4-dimethylphenyl)ethan-1-one (200 mg, 1.35 mmol) and NaBH₄ (51 mg, 1.34 mmol) in MeOH (2 ml) was stirred at RT for 20 min. The reaction was quenched with brine (10 ml) and then extracted with EA (10 ml×2). The organic layer was dried over Na₂SO₄ and concentrated to dryness to give the titled compound (200 mg, 99%), which was used directly for the next step without further purification. ¹H NMR (400 MHz, CDCl₃) δ 7.40 (d, J=7.9 Hz, 1H), 7.05 (d, J=8.2 Hz, 1H), 6.96 (s, 1H), 5.11 (q, J=6.4 Hz, 1H), 2.32 (s, 3H), 2.30 (s, 3H), 1.46 (d, J=6.4 Hz, 3H) ppm.

### Step B: 2-(7-((2S,5R)-4-(1-(2,4-dimethylphenyl)ethyl)-2,5-diethylpiperazin-1-yl)-4-methyl-5-oxo-4,5-dihydro-2H-pyrazolo[4,3-b]pyridin-2-yl)acetonitrile

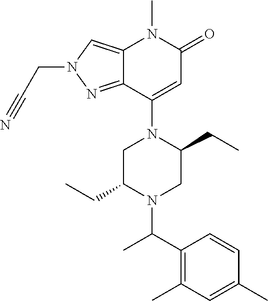 [SMILES: CC[C@H]1CN(C(C)c2ccc(C)cc2C)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (60 mg, 0.18 mmol), 1-(2,4-dimethylphenyl)ethan-1-ol (41,2 mg, 0.27 mmol), (cyanomethyl)trimethylphosphonium iodide (133 mg, 0.55 mmol) and DIPEA (236 mg, 1.83 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography with 0-5% MeOH in DCM and then further purified by Prep-HPLC (Method A) to give the titled compound (23 mg). ¹H NMR (500 MHz, DMSO-d₆) δ 8.32 (s, 0.18H), 7.97 (s, 1H), 7.38 (d, J=7.8 Hz, 0.5H), 7.28 (d, J=7.8 Hz, 0.5H), 6.96 (dd, 1=17.5, 9.0 Hz, 2H), 5.61 (s, 2H), 5.40 (s, 1H), 3.86 (q, J=6.4 Hz, 0.5H), 3.78 (q, J=6.4 Hz, 0.5H), 3.34 (s, 3.5H), 3.27 (s, 3H), 3.12 (s, 0.5H), 3.03 (d, J=10.0 Hz, 0.5H), 2.92 (d, J=11.9 Hz, 0.5H), 2.72 (d, J=11.0 Hz, 0.5H), 2.32 (s, 1.5H), 2.28 (s, 1.5H), 2.24 (d, J=3.6 Hz, 3H), 2.21 (s, 0.5H), 2.06-1.82 (m, 1H), 1.67-1.32 (m, 3H), 1,20 (dd, J=26.6, 6.4 Hz, 3H), 0.92 (dt, J=55, 5 Hz, 3H), 0.58 (dt, J=17.3, 5 Hz, 3H) ppm. MS: M/e 461 (M+1)⁺.

### Compound A7: 2-(7-((2S,5R)-2,5-diethyl-4-(1-(4-ethylphenyl)ethyl)piperazin-1-yl)-4-methyl-5-oxo-4,5-dihydro-2H-pyrazolo[4,3-b]pyridin-2-yl)acetonitrile

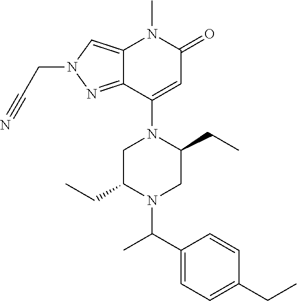 [SMILES: CCc1ccc(C(C)N2C[C@H](CC)N(c3cc(=O)n(C)c4cn(CC#N)nc34)C[C@H]2CC)cc1]

### Step A: 1-(4-ethylphenyl)ethan-1-ol

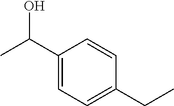

A solution of 1-(4-ethylphenyl)ethan-1-one (200 mg, 1.35 mmol) and NaBH₄ (51 mg, 1.34 mmol) in MeOH (2 ml) was stirred at RT for 60 min. The reaction was quenched with brine (10 ml) and then extracted with EA (10 ml×2). The organic layer was dried over Na₂SO₄ and concentrated to dryness to give the titled compound (200 mg, ⁹⁹⁰/), which was used directly for the next step without further purification. ¹H NMR (500 MHz, CDCl₃) δ 7.30 (d, J=8.0 Hz, 2H), 7.19 (d, J=8.0 Hz, 2H), 4.93-4.82 (m, 1H), 2.65 (q, J=7.6 Hz, 2H), 1.50 (d, J=6.5 Hz, 3H), 1,24 (t, J=7.6 Hz, 3H) ppm.

### Step B: 2-(7-((2S,5R)-2,5-diethyl-4-(1-(4-ethylphenyl)ethyl)piperazin-1-yl)-4-methyl-5-oxo-4,5-dihydro-2H-pyrazolo[4,3-b]pyridin-2-yl)acetonitrile

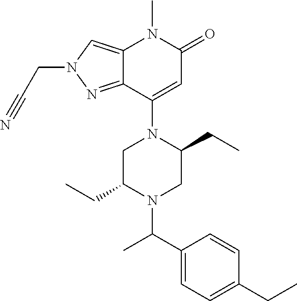 [SMILES: CCc1ccc(C(C)N2C[C@H](CC)N(c3cc(=O)n(C)c4cn(CC#N)nc34)C[C@H]2CC)cc1]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (60 mg, 0.18 mmol), 1-(2,4-dimethylphenyl)ethan-1-ol (41,2 mg, 0.27 mmol), (cyanomethyl)trimethylphosphonium iodide (133 mg, 0.55 mmol) and DIPEA (236 mg, 1.83 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography with 0-5% MeOH in DCM and then Prep-HPLC (Method A) to give the titled compound (31 mg). ¹H NMR (500 MHz, DMSO-d₆) δ 8.21 (s, 0.13H), 7.97 (d, J=1.9 Hz, 1H), 7.26 (dd, J=10.2, 8.1 Hz, 2H), 7.16 (dd, J=11.8, 8.0 Hz, 2H), 5.61 (s, 2H), 5.38 (s, 1H), 3.65 (q, J=6.5 Hz, 0.5H), 3.52 (q, J=6.4 Hz, 0.5H), 3.37 (s, 2H), 3.27 (s, 3H), 3.03 (dd, J=30, 9.6 Hz, 1H), 2.89 (d, J=9.6 Hz, 0.5H), 2.75 (dd, J=30, 9.6 Hz, 0.5H), 2.62-2,54 (m, 2H), 2,47 (s, 1H), 2.31 (s, 0.5H), 2.25 (d, J=12.1 Hz, 0.5H), 2.07-1.96 (m, 0.5H), 1.90-1.79 (m, 0.5H), 1.68-1.36 (m, 3H), 1,21 (ddd, J=31.9, 17.1, 7.0 Hz, 6H), 0.90 (dt, J=39.3, 7.3 Hz, 3H), 0.58 (dt, J=22,5, 15.1 Hz, 3H) ppm. MS: M/e 461 (M+1)⁺.

### Compound A8: 2-(7-((2S,5R)-2,5-diethyl-4-(1-(3-methyl-4-(trifluoromethyl)phenyl)ethyl)piperazin-1-yl)-4-methyl-5-oxo-4,5-dihydro-2H-pyrazolo[4,3-b]pyridin-2-yl)acetonitrile

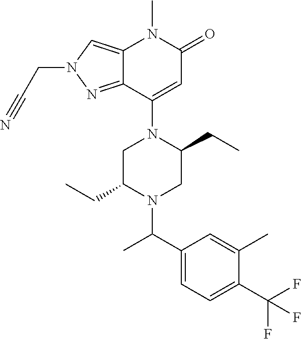 [SMILES: CC[C@H]1CN(C(C)c2ccc(C(F)(F)F)c(C)c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), 1-(3-methyl-4-(trifluoromethyl)phenyl)ethan-1-ol (41 mg, 0.2 mmol) and (cyanomethyl)trimethylphosphonium iodide (85 mg, 0.35 mmol) in CH₃CN (1 mL) was added DIPEA (85 mg, 0.66 mmol). The mixture was stirred at 100° C. in a sealed tube for 16 hours. The mixture was cooled and diluted with EtOAc (10 mL), washed with brine (5 mL×3), dried over Na₂SO₄, filtered and concentrated under reduced pressure. The resulting residue was purified by Prep-TLC to give the titled compound (25 mg, 49%). ¹H NMR (500 MHz, CD₃OD) δ 7.97-7.86 (m, 1H), 7.63-7.52 (m, 1H), 7.46-7.32 (m, 2H), 5.60-5.51 (m, 1H), 5.50-5.41 (m, 2H), 4.83-3.92 (m, 1H), 3.86-3.61 (m, 1H), 3.56-3.32 (m, 4H), 3.30-3.25 (m, 1H), 3.21-2.61 (m, 2H), 2,54-2.27 (m, 4H), 2.20-1.92 (m, 1H), 1.87-1.47 (m, 3H), 1.38-1,29 (m, 3H), 1.10-0.88 (m, 3H), 0.77-0.55 (m, 3H). MS: M/e 515 (M+1)⁺.

### Compound A9: 2-(7-((2S,5R)-2,5-diethyl-4-(1-(4-methoxy-3-methylphenyl)ethyl)piperazin-1-yl)-4-methyl-5-oxo-4,5-dihydro-2H-pyrazolo[4,3-b]pyridin-2-yl)acetonitrile

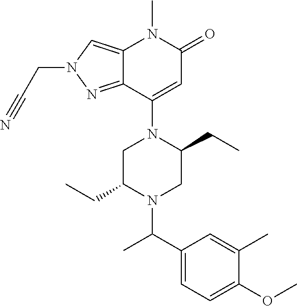 [SMILES: CC[C@H]1CN(C(C)c2ccc(OC)c(C)c2)[C@H](CC)CN1c1cc(=O)n(C)c2cn(CC#N)nc12]

### Step A: 1-(4-methoxy-3-methylphenyl)ethan-1-ol

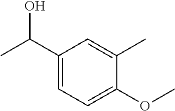

A solution of 1-(4-methoxy-3-methylphenyl)ethan-1-one (200 mg, 1,22 mmol) and NaBH₄ (46 mg, 1,21 mmol) in MeOH (2 ml) was stirred at RT for 60 min. The reaction was quenched with brine (10 ml) and then extracted with EA (10 ml×2). The organic layer was dried over Na₂SO₄ and concentrated to dryness to give the titled compound (200 mg, 99%), which was used directly for the next step without further purification. ¹H NMR (500 MHz, CDCl₃) δ 7.19-7.14 (m, 2H), 6.80 (d, J=9.0 Hz, 1H), 4.83 (q, J=6.4 Hz, 1H), 3.83 (s, 3H), 2.23 (s, 3H), 1.48 (d, J=6.4 Hz, 3H) ppm.

### Step B: 2-(7-((2S,5R)-2,5-diethyl-4-(1-(4-methoxy-3-methylphenyl)ethyl)piperazin-1-yl)-4-methyl-5-oxo-4,5-dihydro-2H-pyrazolo[4,3-b]pyridin-2-yl)acetonitrile

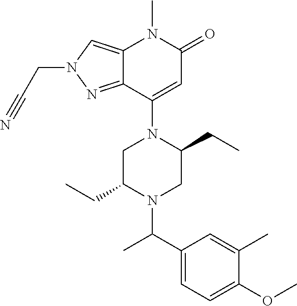 [SMILES: CC[C@H]1CN(C(C)c2ccc(OC)c(C)c2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (60 mg, 0.18 mmol), 1-(4-methoxy-3-methylphenyl)ethan-1-ol (45.6 mg, 0.27 mmol), (cyanomethyl)trimethylphosphonium iodide (133 mg, 0.55 mmol) and DIPEA (236 mg, 1.83 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography with 0-5% MeOH in DCM and then Prep-HPLC (Method A) to give the titled compound (16 mg). ¹H NMR (500 MHz, DMSO-d₆) δ 8.23 (s, 0.2H), 7.97 (d, J=2.8 Hz, 1H), 7.17-7.08 (m, 2H), 6.87 (dd, J=13 1, 8.2 Hz, 1H), 5.61 (s, 2H), 5.38 (d, J=5.7 Hz, 1H), 3.76 (d, J=6.3 Hz, 3H), 3.58 (q, J=6.5 Hz, 0.5H), 3.47 (q, J=6.4 Hz, 0.5H), 3.34 (s, 2H), 3.27 (s, 3H), 3.05 (dd, J=30.3, 10.5 Hz, 1H), 2.89 (d, J=11.4 Hz, 0.5H), 2.76-2.69 (m, 0.5H), 2,45 (d, J=10.6 Hz, 1H), 2.35-2.26 (m, 1H), 2.14 (d, J=8.9 Hz, 3H), 2.06-1.86 (m, 1H), 1.67-1.36 (m, 3H), 1,22 (dd, J=20.2, 6.4 Hz, 3H), 0.90 (dt, J=40.5, 7.2 Hz, 3H), 0.59 (dt, J=20.5, 13.1 Hz, 3H) ppm. MS: M/e 477 (M+1)⁺.

### Compound A10: 2-(7-((2S,5R)-4-(1-(4-(1,1-difluoroethyl)phenyl)ethyl)-2,5-diethylpiperazin-1-yl)-4-methyl-5-oxo-4,5-dihydro-2H-pyrazolo[4,3-b]pyridin-2-yl)acetonitrile

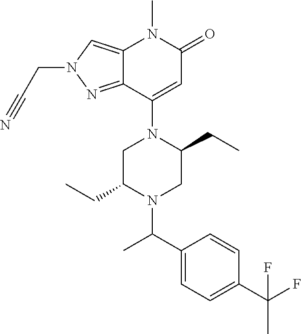 [SMILES: CC[C@H]1CN(C(C)c2ccc(C(C)(F)F)cc2)[C@H](CC)CN1c1cc(=O)n(C)c2cn(CC#N)nc12]

### Step A: 1-(4-(1.1-difluoroethyl)phenyl)ethan-1-one

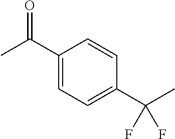

A mixture of 1-bromo-4-(1,1-difluoroethyl)benzene (442 mg, 2 mmol), tributyl(1-ethoxyvinyl)stannane (1.08 g, 3 mmol) and Pd(PPh₃)₂Cl₂ (140 mg, 0.2 mmol) in toluene (5 mL) was stirred at 100° C. under N₂ overnight. To the resulting solution was added HCl (0.5 mL, con.) in drops and the mixture was stirred at room temperature for 30 minutes. The mixture was treated with saturated NaHCO₃ aq. to pH˜8, extracted with EtOAc(10 ml×3), washed with brine (20 mL×3), dried and concentrated to dryness. The resulting residue was purified by flash column chromatography (EtOAc: PE=0-50% in 25 minutes) to give the titled compound (300 mg, 82%).

¹H NMR (400 MHz, CDCl₃) δ 8.01 (d, J=8.2 Hz, 2H), 7.61 (d, J=8.2 Hz, 2H), 2.63 (s, 3H), 1.93 (t, J=18.2 Hz, 3H) ppm. MS: M/e 185 (M+1)⁺.

### Step B: 1-(4-(1.1-difluoroethyl)phenyl)ethan-1-ol

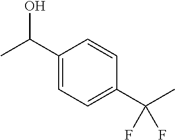

To a solution of 1-(4-(1,1-difluoroethyl)phenyl)ethan-1-one (300 mg, 1.63 mmol) in MeOH (5 mL) was added NaBH₄ (49 mg, 1.3 mmol) at room temperature and the mixture was stirred at room temperature for 30 minutes. The resulting mixture was treated with water, extracted with DCM (10 mL×3). The combined organic layers were washed with brine (20 mL×1), dried over Na₂SO₄ and concentrated to dryness. The resulting residue (250 mg, crude) was used in the next step. MS: M/e 187 (M+1)⁺.

### Step C: 2-(7-((2S,5R)-4-(1-(4441.1-difluoroethyl)phenyl)ethyl)-2,5-diethylpiperazin-1-yl-4-methyl-5-oxo-4,5-dihydro-2H-pyrazolo[4,3-b]pyridin-2-yl)acetonitrile

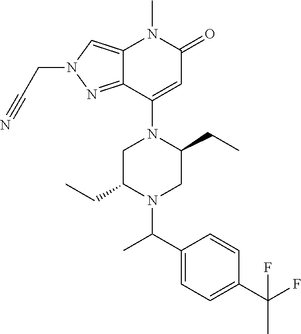 [SMILES: CC[C@H]1CN(C(C)c2ccc(C(C)(F)F)cc2)[C@H](CC)CN1c1cc(=O)n(C)c2cn(CC#N)nc12]

A mixture of 1-(4-(1,1-difluoroethyl)phenyl)ethan-1-ol (28 mg, 0.15 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (48 mg, 0.2 mmol) and DIPEA (51 mg, 0.4 mmol) in MeCN (2 mL) was stirred at 100° C. overnight. The reaction mixture was diluted with EtOAc (10 mL), washed with brine (10 mL×2), dried over Na₂SO₄ and concentrated to dryness. The resulting residue was purified by column chromatography (MeOH: DCM=0-10% in 20 minutes) to give the titled compound (18 mg, 36%). ¹H NMR (500 MHz, DMSO-d₆) δ 7.98 (d, J=1.4 Hz, 1H), 7.50 (dt, J=24.9, 8.1 Hz, 4H), 5.61 (d, J=2.8 Hz, 2H), 5.39 (s, 1H), 3.81-3.61 (m, 1H), 3.41-3.32 (m, 1H), 3.28 (d, J=13.2 Hz, 4H), 3.15-3.02 (m, 1H), 2.95-2.74 (m, 1H), 2,54 (s, 1H), 2.32-2.17 (m, 1H), 2.08-1.79 (m, 4H), 1.69-1.41 (m, 3H), 1,26 (dd, J=15.7, 6.5 Hz, 3H), 0.98-0.82 (m, 3H), 0.65-0.49 (m, 3H) ppm. MS: M/e 497 (M+1)⁺.

Compound A1l: 2-(7-((2S,5R)-2,5-diethyl-4-(1-(4-(2-methoxyethoxy)phenyl)ethyl)piperazin-1-yl)-4-methyl-5-obo-4,5-dihydro-2H-pyrazolo[4,3-b]pyridin-2-yl)acetonitrile

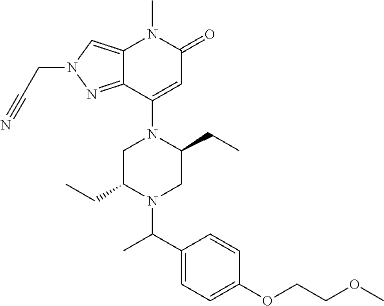 [SMILES: CC[C@H]1CN(C(C)c2ccc(OCCOC)cc2)[C@H](CC)CN1c1cc(=O)n(C)c2cn(CC#N)nc12]

### Step A: 1-(4-(2-methoxyethoxy)phenyl)ethan-1-one

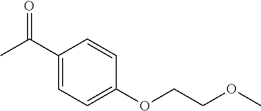

To a solution of 1-(4-hydroxyphenyl)ethan-1-one (270 mg, 2 mmol) in CH₃CN (10 mL) was added 1-bromo-2-methoxyethane (417 mg, 3 mmol) and K₂CO₃ (552 mg, 4 mmol). The reaction mixture was stirred at 70° C. in a sealed bottle overnight. Another portion of 1-bromo-2-methoxyethane (417 mg, 3 mmol) was added and the resulting mixture was heated at 70° C. for 5 hours. The mixture was cooled down to RT, diluted with H₂O, extracted with EA (80 mL×2), washed with brine, dried over Na₂SO₄, filtered, and concentrated to dryness. The resulting residue was purified by flash column chromatography to give the titled compound (330 mg, 85%). MS: M/e 195 (M+1)⁺.

### Step B: 1-(4-(2-methoxyethoxy)phenyl)ethan-1-ol

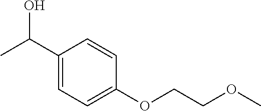

To a solution of 1-(4-(2-methoxyethoxy)phenyl)ethan-1-one (330 mg, 1.7 mmol) in MeOH (5 mL) was added NaBH₄ (65 mg, 1.7 mmol) slowly at 0° C. The resulting mixture was stirred at room temperature for 2 hours. The reaction mixture was quenched with H₂O (10 mL) and extracted with EA (80 mL), washed with brine, dried over Na₂SO₄, filtered, and concentrated to dryness. The resulting residue was purified by flash column chromatography to give the desired product (160 mg, 48%). ¹H NMR (400 MHz, DMSO-d₆) δ 7.23 (d, J=8.4 Hz, 2H), 6.86 (d, J=8.0 Hz, 2H), 5.01 (d, J=4.0 Hz, 1H), 4.70-4.58 (m, 1H), 4.05 (t, J=4.8 Hz, 2H), 3.64 (t, J=4.8 Hz, 2H), 3.32 (s, 3H), 1,28 (d, J=6.4 Hz, 3H) ppm.

### Step C: 2-(7-((2S,5R)-2,5-diethyl-4-(1-(4-(2-methoxyethoxy)phenyl)ethyl)piperazin-1-yl-4-methyl-5-oxo-4,5-dihydro-2H-pyrazolo[4,3-b]pyridin-2-yl)acetonitrile

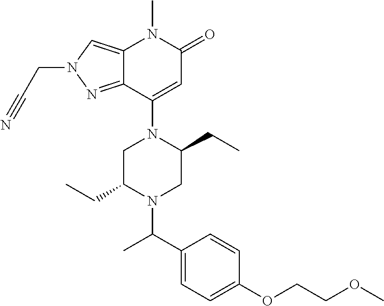 [SMILES: CC[C@H]1CN(C(C)c2ccc(OCCOC)cc2)[C@H](CC)CN1c1cc(=O)n(C)c2cn(CC#N)nc12]

To a solution of 1-(4-(2-methoxyethoxy)phenyl)ethan-1-ol (39 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-TLC(DCM/MeOH=25/1) and then Prep-HPLC (Method A) to give the titled compound (5 mg, 10%). ¹H NMR (400 MHz, CD₃OD) δ 7.93-7.90 (m, 1H), 7.33-7.24 (m, 2H), 6.94-6.86 (m, 2H), 5.54 (s, 1H), 5.48-5.44 (m, 2H), 4.14-4.06 (m, 2H), 3.78-3.72 (m, 2H), 3.71-3.65 (m, 0.5H), 3.60-3.40 (m, 7.5H), 3.35-3.31 (m, 2H), 3.18-3.07 (m, 0.5H), 3.02-2.95 (m, 0.5H), 2.89-2.80 (m, 0.5H), 2.64-2,56 (m, 0.5H), 2,50-2.38 (m, 1H), 2.15-1.45 (m, 4H), 1.36-1,26 (m, 3H), 1.05-0.91 (m, 3H), 0.75-0.55 (m, 3H) ppm. MS: M/e 507 (M+1)⁺.

### Compound A12: 2-(7-((2S,5R)-2,5-diethyl-4-(1-(2-fluoro-4-methoxyphenyl)ethyl)piperazin-1-yl)-4-methyl-5-oxo-4,5-dihydro-2H-pyrazolo[4,3-b]pyridin-2-yl)acetonitrile

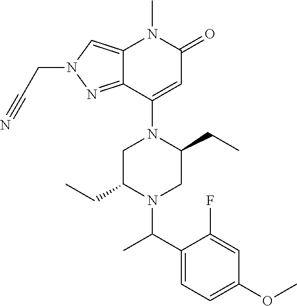 [SMILES: CC[C@H]1CN(C(C)c2ccc(OC)cc2F)[C@H](CC)CN1c1cc(=O)n(C)c2cn(CC#N)nc12]

### Step A: 1-(2-fluoro-4-methoxyphenyl)ethan-1-ol

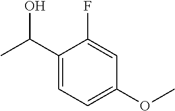

To a solution of 1-(2-fluoro-4-methoxyphenyl)ethan-1-one (336 mg, 2 mmol) in MeOH (10 mL) was added NaBH₄ (76 mg, 2 mmol) slowly at 0° C. The resulting mixture was stirred at room temperature for 2 hours. The reaction mixture was quenched with H₂O (20 mL) and extracted with EA (80 mL), washed with brine, dried over Na₂SO₄, filtered, and concentrated to dryness. The resulting residue was purified by flash column chromatography to give the desired product (300 mg, 87%). ¹H NMR (500 MHz, DMSO-d₆) δ 7.39 (t, J=8.5 Hz, 1H), 6.79-6.70 (m, 2H), 5.15 (d, J=4.5 Hz, 1H), 4.95-4.86 (m, 1H), 3.74 (s, 3H), 1.30 (d, J=6.5 Hz, 3H) ppm.

### Step B: 2-(7-((2S,5R)-2,5-diethyl-4-(1-(2-fluoro-4-methoxyphenyl)ethyl)piperazin-1-yl)-4-methyl-5-oxo-4,5-dihydro-2H-pyrazolo[4,3-b]pyridin-2-yl)acetonitrile

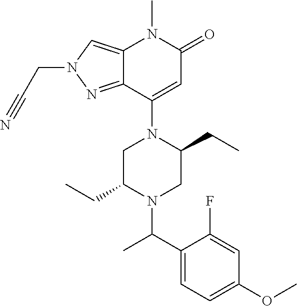 [SMILES: CC[C@H]1CN(C(C)c2ccc(OC)cc2F)[C@H](CC)CN1c1cc(=O)n(C)c2cn(CC#N)nc12]

To a solution of 1-(2-fluoro-4-methoxyphenyl)ethan-1-ol (33 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (75 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-TLC (DCM/MeOH=20/1) and then Prep-HPLC (Method A) to give the titled compound (2 mg, 4%). ¹H NMR (500 MHz, CD₃OD) δ 7.91-7.89 (m, 1H), 7.51-7.37 (m, 1H), 6.82-6.60 (m, 2H), 5.54 (s, 1H), 5.49-5.44 (m, 2H), 4.12-4.04 (m, 0.5H), 3.98-3.90 (m, 0.5H), 3.80 (s, 1.5H), 3.78 (s, 1.5H), 3.51-3.40 (m, 4H), 3.35-3.31 (m, 2H), 3.18-3.08 (m, 0.5H), 2.97-2.84 (m, 1 H), 2.70-2.60 (m, 0.5H), 2,49-2.36 (m, 1H), 2.21-1.40 (m, 4H), 1.38-1,26 (m, 3H), 1.05-0.91 (m, 3H), 0.78-0.61 (m, 3H) ppm. MS: M/e 481 (M+1)⁺.

### Compound A13: 2-(7-((2S,5R)-2,5-diethyl-4-(1-(2-fluoro-4-((tetrahydrofuran-3-yl)oxy)phenyl)ethyl)piperazin-1-yl)-4-methyl-5-oxo-4,5-dihydro-2H-pyrazolo[4,3-b]pyridin-2-yl)acetonitrile

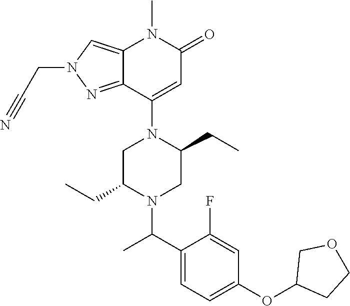 [SMILES: CC[C@H]1CN(C(C)c2ccc(OC3CCOC3)cc2F)[C@H](CC)CN1c1cc(=O)n(C)c2cn(CC#N)nc12]

### Step A: 1-(2-fluoro-4-((tetrahydrofuran-3-yl)oxy)phenyl)ethan-1-one

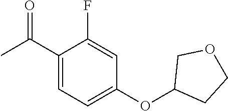 [SMILES: CC(=O)c1ccc(OC2CCOC2)cc1F]

To a solution of 1-(2-fluoro-4-hydroxyphenyl)ethan-1-one (462 mg, 3 mmol) in CH₃CN (10 mL) was added tetrahydrofuran-3-yl 4-methylbenzenesulfonate (363 mg, 1.5 mmol) and K₂CO₃ (820 mg, 6 mmol). The reaction mixture was stirred at 90° C. in a sealed bottle overnight. The mixture was cooled down to RT, diluted with H₂O, extracted with EA (60 mL×2), washed with brine, dried over Na₂SO₄, filtered, and concentrated to dryness. The resulting residue was purified by flash column chromatography to give the titled compound (130 mg). MS: M/e 225 (M+1)⁺.

### Step B: 1-(2-fluoro-4-((tetrahydrofuran-3-yl)oxy)phenyl)ethan-1-ol

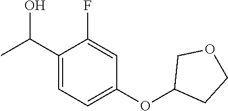 [SMILES: CC(O)c1ccc(OC2CCOC2)cc1F]

To a solution of 1-(2-fluoro-4-((tetrahydrofuran-3-yl)oxy)phenyl)ethan-1-one (130 mg, 0.58 mmol) in MeOH (5 mL) was added NaBH₄ (44 mg, 1.16 mmol) slowly at 0° C. The resulting mixture was stirred at room temperature for 1 hour. The reaction mixture was quenched with H₂O (10 mL) and extracted with EA (80 mL), washed with brine, dried over Na₂SO₄, filtered, and concentrated to give the desired product (110 mg, 84%). ¹H NMR (400 MHz, DMSO-d₆) δ 7.39 (t, J=8.5 Hz, 1H), 6.78-6.69 (m, 2H), 5.15 (d, J=4.5 Hz, 1H), 5.04-4.98 (m, 1H), 4.94-4.85 (m, 1H), 3.90-3.70 (m, 4H), 2.26-2.17 (m, 1H), 1.98-1.89 (m, 1H), 1.30 (d, 0.1=6.5 Hz, 3H) ppm.

### Step C: 2-(7-((2S,5R)-2,5-diethyl-4-(1-(2-fluoro-4-((tetrahydrofuran-3-yl)oxy)phenyl))ethyl)piperazin-1-yl)-4-methyl-5-oxo-4,5-dihydro-2H-pyrazolo[4,3-b]pyridin-2-yl)acetonitrile

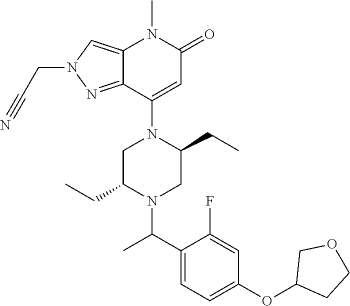 [SMILES: CC[C@H]1CN(C(C)c2ccc(OC3CCOC3)cc2F)[C@H](CC)CN1c1cc(=O)n(C)c2cn(CC#N)nc12]

To a solution of 1-(2-fluoro-4-((tetrahydrofuran-3-yl)oxy)phenyl)ethan-1-ol (45 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-HPLC (Method A) to give the titled compound (8 mg, 14%). ¹H NMR (500 MHz, CD₃OD) δ 7.93-7.91 (m, 1H), 7.52-7.37 (m, 1H), 6.80-6.60 (m, 2H), 5.54 (s, 1H), 5.48-5.44 (m, 2H), 5.05-4.95 (m, 1H), 4.12-4.04 (m, 0.5H), 4.00-3.83 (m, 4.5H), 3.51-3.44 (m, 0.5H), 3.43 (s, 3H), 3.35-3.31 (m, 2H), 3.29-3.24 (m, 0.5H), 3.15-3.06 (m, 0.5H), 2.96-2.84 (m, 1H), 2.69-2.61 (m, 0.5H), 2,48-2.36 (m, 1H), 2.31-2.20 (m, 1H), 2.15-1.90 (m, 2H), 1.85-1.45 (m, 3H), 1.35 (d, J=6.5 Hz, 1.5H), 1.30 (d, J=6.5 Hz, 1.5H), 1.02 (t, J=7.5 Hz, 1.5H), 0.95 (t, J=7.5 Hz, 1.5H), 0.74 (t, J=7.5 Hz, 1.5H), 0.65(t, J=7.5 Hz, 1.5H) ppm. MS: M/e 537 (M+1)⁺.

### Compound A14: 2-(7-((2S,5R)-2,5-diethyl-4-(1-(4-methoxy-2-methylphenyl)ethyl)piperazin-1-yl)-4-methyl-5-oxo-4,5-dihydro-2H-pyrazolo[4,3-b]pyridin-2-yl)acetonitrile

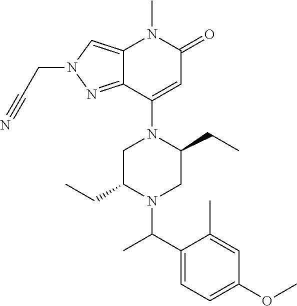 [SMILES: CC[C@H]1CN(C(C)c2ccc(OC)cc2C)[C@H](CC)CN1c1cc(=O)n(C)c2cn(CC#N)nc12]

### Step A: 1-(4-methoxy-2-methylphenyl)ethan-1-one

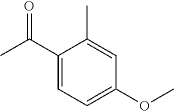

To a solution of 1-(4-hydroxy-2-methylphenyl)ethan-1-one (450 mg, 3 mmol) and CH₃I (511 mg, 3.6 mmol) in acetone (5 mL) was added K₂CO₃ (828 mg, 6 mmol). The reaction mixture was stirred at 90° C. under N₂ overnight. The mixture was cooled to room temperature, diluted with water, extracted with EA (80 mL), washed with brine, dried over Na₂SO₄, filtered, and concentrated to give the titled compound (420 mg, 84%). MS: M/e 165 (M+1)⁺.

### Step B: 1-(4-methoxy-2-methylphenyl)ethan-1-ol

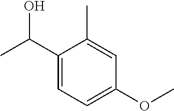

To a solution of 1-(4-methoxy-2-methylphenyl)ethan-1-one (420 mg, 2,56 mmol) in MeOH (10 mL) was added NaBH₄ (200 mg, 5.26 mmol) slowly at 0° C. The resulting mixture was stirred at room temperature for 3 hours. The reaction mixture was quenched with H₂O (10 mL) and extracted with EA (80 mL), washed with brine, dried over Na₂SO₄, filtered, and concentrated to dryness. The resulting residue was purified by flash column chromatography to give the desired product (360 mg, 84%).

### Step C: 2-(7-((2S,5R)-2,5-diethyl-4-(1-(4-methoxy-2-methylphenyl)ethyl)piperazin-1-yl-4-methyl-5-oxo-4,5-dihydro-2H-pyrazolo[4,3-b]pyridin-2-yl)acetonitrile

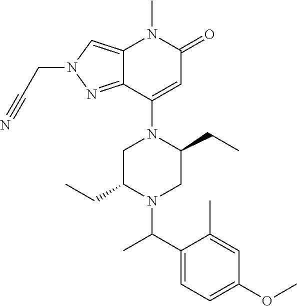 [SMILES: CC[C@H]1CN(C(C)c2ccc(OC)cc2C)[C@H](CC)CN1c1cc(=O)n(C)c2cn(CC#N)nc12]

To a solution of 1-(4-methoxy-2-methylphenyl)ethan-1-ol (34 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (75 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-HPLC (Method A) to give the titled compound (14 mg, 29%). ¹H NMR (500 MHz, CD₃OD) δ 7.91-7.89 (m, 1H), 7.48-7.32 (m, 1H), 6.77-6.66 (m, 2H), 5.57-5.52 (m, 1H), 5.49-5.44 (m, 2H), 3.95-3.80 (m, 1H), 3.76 (s, 3H), 3.52-3.44 (m, 0.5H), 3.43 (s, 3H), 3.35-3.31 (m, 2H), 3.18-3.10 (m, 1H), 3.05-3.00 (m, 0.5H), 2.88-2.78 (m, 0.5H), 2.66-2,56 (m, 0.5H), 2,52-2,44 (m, 0.5H), 2,42-2.31 (m, 3.5H), 2.21-1.40 (m, 4H), 1.32-1,21 (m, 3H), 1.05-0.87 (m, 3H), 0.72-0.58 (m, 3H) ppm. MS: M/e 477 (M+1)⁺.

### Compound A15: 2-(7-((2S,5R)-2,5-diethyl-4-(1-(4-(((tetrahydro-2H-pyran-4-yl)oxy)methyl)phenyl)ethyl)piperazin-1-yl)-4-methyl-5-oxo-4,5-dihydro-2H-pyrazolo[4,3-b]pyridin-2-yl)acetonitrile

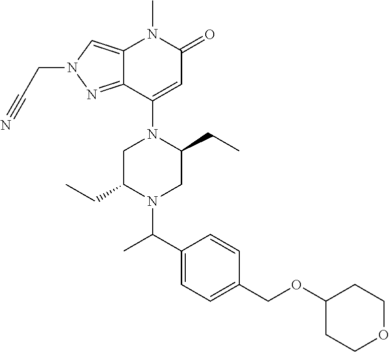 [SMILES: CC[C@H]1CN(C(C)c2ccc(COC3CCOCC3)cc2)[C@H](CC)CN1c1cc(=O)n(C)c2cn(CC#N)nc12]

### Step A: 1-(4-(((tetrahydro-2H-pyran-4-yl)oxy)methyl)phenyl)ethan-1-one

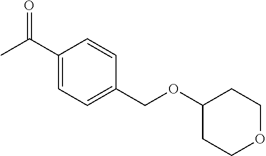 [SMILES: CC(=O)c1ccc(COC2CCOCC2)cc1]

To a solution of 1-(4-(bromomethyl)phenyl)ethan-1-one (300 mg, 1.42 mmol) in DMF (3 mL) was added tetrahydro-2H-pyran-4-ol (173 mg, 1.70 mmol), TBAB (46 mg, 0.14 mmol) and Cs₂CO₃ (923 mg, 2.83 mmol). The resulting mixture was stirred at 80° C. overnight. The reaction mixture was diluted with DCM (200 mL). The organic layer was washed with water, dried over Na₂SO₄ and concentrated to give the titled compound (290 mg, 88%). MS: M/e 235 (M+1)⁺.

### Step B: 1-(4-(((tetrahydro-2H-pyran-4-yl)oxy)methyl)phenyl)ethan-1-ol

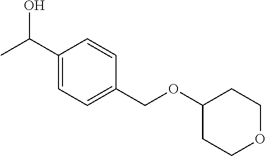 [SMILES: CC(O)c1ccc(COC2CCOCC2)cc1]

To a solution of 1-(4-(((tetrahydro-2H-pyran-4-yl)oxy)methyl)phenyl)ethan-1-one (290 mg, 1,24 mmol) in MeOH (15 mL) was added NaBH₄ (94 mg, 2,48 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (270 mg, 92%). MS: M/e 237 (M+1)⁺.

### Step C: 2-(7-((2S,5R)-2,5-diethyl-4-(1-(4-(((tetrahydro-2H-pyran-4-yl)oxy)methyl)phenyl)ethyl)piperazin-1-yl)-4-methyl-5-oxo-4,5-dihydro-2H-pyrazolo[4,3-b]pyridin-2-yl)acetonitrile

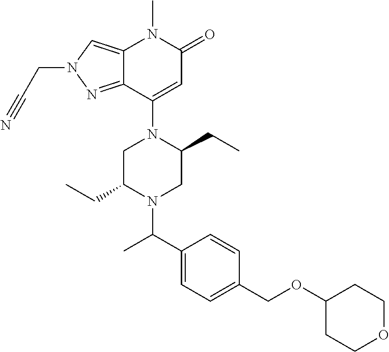 [SMILES: CC[C@H]1CN(C(C)c2ccc(COC3CCOCC3)cc2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(4-(((tetrahydro-2H-pyran-4-yl)oxy)methyl)phenyl)ethan-1-ol (36 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled compound(19 mg, 23%). ¹H NMR (400 MHz, CD₃OD) δ 7.91 (d, J=3.6 Hz, 1H), 7.38 (d, J=8.0 Hz, 1H), 7.36-7.29 (m, 3H), 5.55 (s, 1H), 5.46 (d, J=5.0 Hz, 2H), 4.57 (d, J=4.3 Hz, 2H), 3.95-3.89 (m, 2H), 3.77-3.44 (m, 5H), 3.43 (s, 3H), 3.30-3.09 (m, 3H), 3.02-2.37 (m, 2H), 2.11-1.83 (m, 3H), 1.58 (ddd, J=40.3, 23.4, 7.2 Hz, 5H), 1.32 (dd, J=16.0, 6.5 Hz, 3H), 0.99 (dt, J=29.9, 7.3 Hz, 3H), 0.64 (dt, J=29.9, 7.4 Hz, 3H) ppm. MS: M/e 547 (M+1)⁺.

### Compound A16: 2-(7-((2S,5R)-2,5-diethyl-4-(1-(4-((oxetan-3-yloxy)methyl)phenyl)ethyl)piperazin-1-yl)-4-methyl-5-oxo-4,5-dihydro-2H-pyrazolo[4,3-b]pyridin-2-yl)acetonitrile

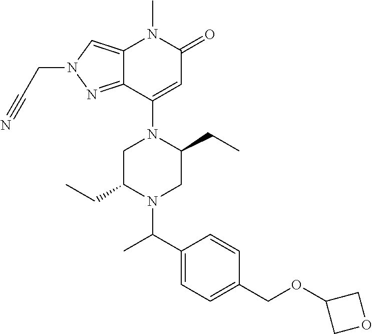 [SMILES: CC[C@H]1CN(C(C)c2ccc(COC3COC3)cc2)[C@H](CC)CN1c1cc(=O)n(C)c2cn(CC#N)nc12]

### Step A: 1-(4-((oxetan-3-yloxy)methyl)phenyl)ethan-1-one

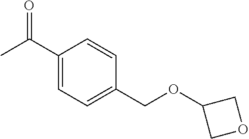

To a solution of 1-(4-(bromomethyl)phenyl)ethan-1-one (300 mg, 1.42 mmol) in DMF (3 mL) was added oxetan-3-ol (173 mg, 1.70 mmol), TBAB (46 mg, 0.14 mmol) and Cs₂CO₃ (923 mg, 2.83 mmol). The resulting mixture was stirred at 80° C. overnight. The reaction mixture was diluted with DCM (200 mL). The organic layer was washed with water, dried over Na₂SO₄ and concentrated to give the titled compound (230 mg, 79%). MS: M/e 207 (M+1)⁺.

### Step B: 1-(4-((oxetan-3-yloxy)methyl)phenyl)ethan-1-ol

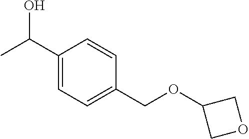

To a solution of 1-(4-((oxetan-3-yloxy)methyl)phenyl)ethan-1-one (230 mg, 1.12 mmol) in MeOH (15 mL) was added NaBH₄ (85 mg, 2.23 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (200 mg, 86%). MS: M/e 209 (M+1)⁺.

### Step C: 2-(7-((2S,5R)-2,5-diethyl-4-(1-(4-((oxetan-3-yloxy)methyl)phenyl)ethyl)piperazin-1-yl)-4-methyl-5-oxo-4,5-dihydro-2H-pyrazolo[4,3-b]pyridin-2-yl)acetonitrile

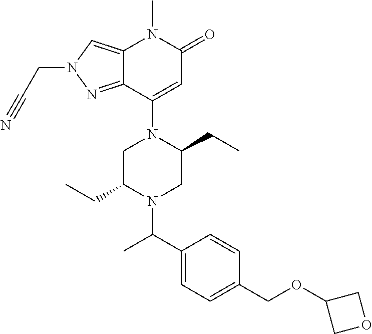 [SMILES: CC[C@H]1CN(C(C)c2ccc(COC3COC3)cc2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(4-((oxetan-3-yloxy)methyl)phenyl)ethan-1-ol (32 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled compound (20 mg, 25%). ¹H NMR (400 MHz, CD₃OD) δ 7.91 (d, J=3.4 Hz, 1H), 7.39 (d, J=7.9 Hz, 1H), 7.38-7.30 (m, 3H), 5.55 (s, 1H), 5.46 (d, J=4.9 Hz, 2H), 4.83-4.65 (m, 4H), 4.55-4.51 (m, 2H), 4.47 (d, J=4.5 Hz, 2H), 3.76-3.58 (m, 1H), 3.54-3.44 (m, 1H), 3.43 (s, 3H), 3.27-2.96 (m, 2H), 2.90-2,57 (m, 1H), 2,48-2.32 (m, 1H), 2.17-1.41 (m, 4H), 1.32 (dd, J=17.1, 6.5 Hz, 3H), 0.99 (dt, J=29.4, 7.4 Hz, 3H), 0.65 (dt, J=25.5, 7.2 Hz, 3H) ppm. MS: M/e 519 (M+1)⁺.

### Compound A17: 2-(7-((2S,5R)-2,5-diethyl-4-(1-(4-(((tetrahydrofuran-3-yl)oxy)methyl)phenyl)ethyl)piperazin-1-yl)-4-methyl-5-oxo-4,5-dihydro-2H-pyrazolo[4,3-b]pyridin-2-yl)acetonitrile

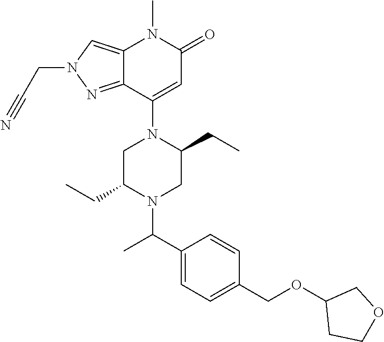 [SMILES: CC[C@H]1CN(C(C)c2ccc(COC3CCOC3)cc2)[C@H](CC)CN1c1cc(=O)n(C)c2cn(CC#N)nc12]

### Step A: 1-(4-(((tetrahydrofuran-3-yl)oxy)methyl)phenyl)ethan-1-one

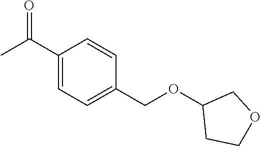 [SMILES: CC(=O)c1ccc(COC2CCOC2)cc1]

To a solution of 1-(4-(bromomethyl)phenyl)ethan-1-one (300 mg, 1.42 mmol) in DMF (3 mL) was added tetrahydrofuran-3-ol (149 mg, 1.70 mmol), TBAB (46 mg, 0.14 mmol) and Cs₂CO₃ (923 mg, 2.83 mmol). The resulting mixture was stirred at 80° C. overnight. The reaction mixture was diluted with DCM (200 mL). The organic layer was washed with water, dried over Na₂SO₄ and concentrated to give the titled compound (270 mg, 87%). MS: We 221 (M+1)⁺.

### Step B: 1-(4-(((tetrahydrofuran-3-yl)oxy)methyl)phenyl)ethan-1-ol

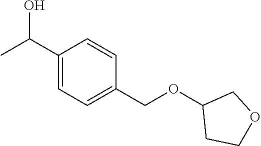 [SMILES: CC(O)c1ccc(COC2CCOC2)cc1]

To a solution of 1-(4-(((tetrahydrofuran-3-yl)oxy)methyl)phenyl)ethan-1-one (270 mg, 1,23 mmol) in MeOH (15 mL) was added NaBH₄ (94 mg, 2,45 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (250 mg, 92%). MS: M/e 223 (M+1)⁺.

### Step C: 2-(7-((2S,5R)-2,5-diethyl-4-(1-(4-(((tetrahydrofuran-3-yl)oxy)methyl)phenyl)ethyl)piperazin-1-yl)-4-methyl-5-oxo-4,5-dihydro-2H-pyrazolo[4,3-b]pyridin-2-yl)acetonitrile

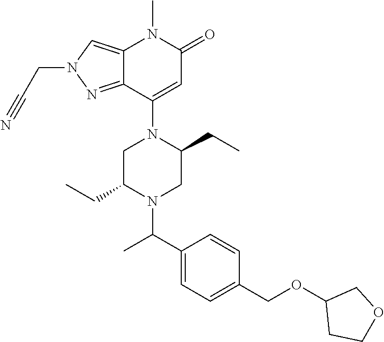 [SMILES: CC[C@H]1CN(C(C)c2ccc(COC3CCOC3)cc2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(4-(((tetrahydrofuran-3-yl)oxy)methyl)phenyl)ethan-1-ol (34 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled compound(28 mg, 34%). ¹H NMR (400 MHz, CD₃OD) δ 7.91 (d, J=3.5 Hz, 1H), 7.38 (d, J=8.0 Hz, 1H), 7.36-7.28 (m, 3H), 5.55 (s, 1H), 5.46 (d, J=4.8 Hz, 2H), 4.54-4.46 (m, 2H), 4.29-4.24 (m, 1H), 3.90-3.74 (m, 4H), 3.62-3.50 (m, 1H), 3.49-3.43 (m, 1H), 3.43 (d, J=1,2 Hz, 3H), 3.29-3.24 (m, 2H), 3.19-2,59 (m, 2H), 2,48-2.34 (m, 1H), 2.17-1.92 (m, 3H), 1.85-1.46 (m, 3H), 1.32 (dd, J=16.2, 6.5 Hz, 3H), 0.99 (dt, J=29.5, 7.2 Hz, 3H), 0.65 (dt, J=29.2, 7.3 Hz, 3H) ppm. MS: We 533 (M+1)⁺.

### Compound A18: 2-(7-((2S,5R)-2,5-diethyl-4-(1-(4-((2-methoxyethoxy)methyl)phenyl)ethyl)piperazin-1-yl)-4-methyl-5-oxo-4,5-dihydro-2H-pyrazolo[4,3-b]pyridin-2-yl)acetonitrile

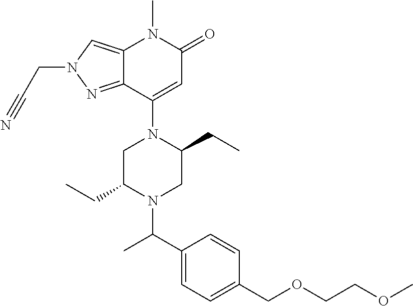 [SMILES: CC[C@H]1CN(C(C)c2ccc(COCCOC)cc2)[C@H](CC)CN1c1cc(=O)n(C)c2cn(CC#N)nc12]

### Step A: methyl 4-(1-((tert-butyldimethylsilyl)oxy)ethyl)benzoate

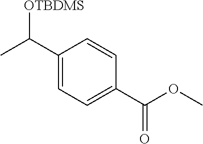 [SMILES: COC(=O)c1ccc(C(C)O[Si](C)(C)C(C)(C)C)cc1]

To a stirred solution of methyl 4-(1-hydroxyethyl)benzoate (5 g, 27.78 mmol) and imidazole (2.27 g, 33.33 mmol) in DCM (200 mL) was added TBDMSCl (4.17 g, 27.78 mmol). After then, the mixture was stirred for 3 hrs. The mixture was diluted with CH₂Cl₂ (20 mL), then washed with H₂O, brine, dried over Na₂SO₄, concentrated to dryness. The resulting residue was purified by flash column chromatography to give the titled compound (8 g, 98%). MS: M/e 295 (M+1)⁺.

### Step B: (4-(1-((tert-butyldimethylsilyl)oxyethyl)phenyl)methanol

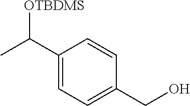 [SMILES: CC(O[Si](C)(C)C(C)(C)C)c1ccc(CO)cc1]

To a stirred solution of methyl 4-(1-((tert-butyldimethylsilyl)oxy)ethyl)benzoate (8 g, 27.21 mmol) in THF (200 mL) was added aluminum(III) lithium hydride (1.0 M, 32.65 mmol, 32.65 mL) at 0° C. After then, the mixture was stirred at RT for 5 hours. The mixture was quenched with a.q NaOH, filtered and extracted with CH₂Cl₂. The combined organic layers were washed with brine, dried over Na₂SO₄, concentrated to give the titled compound (6.7 g, 92%). MS: M/e 267 (M+1)⁺.

### Step C: tert-butyl(1-(4-((2-methoxyethoxy)methyl)phenyl)ethoxy)dimethylsilane

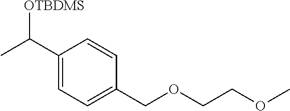 [SMILES: COCCOCc1ccc(C(C)O[Si](C)(C)C(C)(C)C)cc1]

To a mixture of (4-(1-((tert-butyldimethylsilyl)oxy)ethyl)phenyl)methanol (200 mg, 0.75 mmol) in THF (20 mL) was added NaH (36 mg, 0.90 mmol) at 0° C. and stirred for 1 h, Then 1-bromo-2-methoxyethane (156 mg, 1.13 mmol) was added and stirred at RT for 2 hrs. The reaction mixture was extracted with EA and washed with brine, dried over Na₂SO₄, concentrated to dryness. The resulting residue was purified by flash column chromatography to give the titled compound (170 mg, 70%). MS: M/e 325 (M+1)⁺.

### Step D: 1-(4-((2-methoxyethoxy)methyl)phenyl)ethan-1-ol

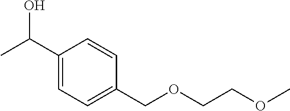

To a solution of tert-butyl(1-(4-(methoxymethyl)phenyl)ethoxy)dimethylsilane (170 mg, 0.52 mmol) in THF (5 mL) was added TBAF (1 mL, 1 mmol). The reaction mixture was stirred at room for 3 hours. The reaction mixture was diluted with water and extracted with EA (80 mL×2). The combined organic layers were washed with brine, dried over Na₂SO₄, filtered and concentrated to dryness. The resulting residue was purified by flash column chromatography to give the titled compound (80 mg, 72%). MS: M/e 211 (M+1)⁺.

### Step E: 2-(7-((2S,5R)-2,5-diethyl-4-(1-(4-((2-methoxyethoxy)methyl)phenyl)ethyl)piperazin-1-yl)-4-methyl-5-oxo-4,5-dihydro-2H-pyrazolo[4,3-b]pyridin-2-yl)acetonitrile

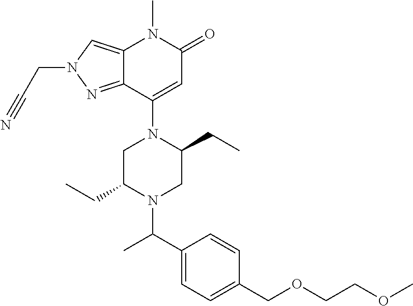 [SMILES: CC[C@H]1CN(C(C)c2ccc(COCCOC)cc2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(4-((2-methoxyethoxy)methyl)phenyl)ethan-1-ol (32 mg, 0.18 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled compound(32 mg, 40%). ¹H NMR (400 MHz, CD₃OD) δ 7.92 (d, 0.1=2.3 Hz, 1H), 7.39 (d, J=8.1 Hz, 1H), 7.37-7.30 (m, 3H), 5.55 (s, 1H), 5.46 (d, J=4.3 Hz, 2H), 4.54 (d, J=6.1 Hz, 2H), 3.88-3.64 (m, 1H), 3.64-3.59 (m, 2H), 3.59-3.56 (m, 2H), 3.54-3.44 (m, 1H), 3.43 (s, 3H), 3.36 (d, J=2.8 Hz, 3H), 3.28-3.01 (m, 3H), 2.93-2.39 (m, 2H), 2.18-1.87 (m, 1H), 1.84-1.49 (m, 3H), 1.35 (dd, J=22.7, 6.4 Hz, 3H), 0.99 (dt, J=28.1, 7.4 Hz, 3H), 0.65 (dt, J=32.0, 7.4 Hz, 3H) ppm. MS: M/e 521 (M+1)⁺.

### Compound A19: 2-(7-((2S,5R)-4-(1-(4-(difluoromethoxy)phenyl)ethyl)-2,5-diethylpiperazin-1-yl)-4-methyl-5-oxo-4,5-dihydro-2H-pyrazolo[4,3-b]pyridin-2-yl)acetonitrile

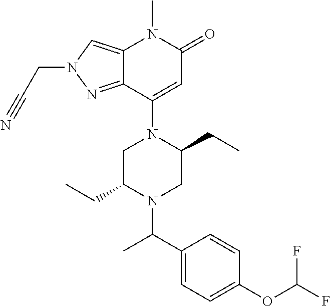 [SMILES: CC[C@H]1CN(C(C)c2ccc(OC(F)F)cc2)[C@H](CC)CN1c1cc(=O)n(C)c2cn(CC#N)nc12]

### Step A: 1-(4-(difluoromethoxy)phenyl)ethan-1-ol

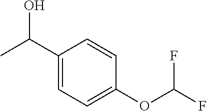

To a solution of 1-(4-(difluoromethoxy)phenyl)ethan-1-one (558 mg, 3 mmol) in MeOH (10 mL) was added NaBH₄ (114 mg, 3 mmol) slowly at 0° C. The resulting mixture was stirred at room temperature for 2 hours. The reaction mixture was quenched with H₂O (20 mL), extracted with EA (80 mL), washed with brine, dried over Na₂SO₄, filtered, and concentrated to dryness. The resulting residue was used directly for next step (500 mg, 88%). ¹H NMR (500 MHz, DMSO-d₆) δ 7.41-7.35 (m, 2H), 7.11 (d, J=8.5 Hz, 2H), 7.34-7.02 (m, 1H), 5.18 (d, J=4.5 Hz, 1H), 4.76-4.68 (m, 1H), 1.30 (d, J=6.5 Hz, 3H) ppm.

### Step B: 2-(7-((2S,5R)-4-(1-(4-(difluoromethoxy)phenyl)ethyl)-2,5-diethylpiperazin-1-yl-4-methyl-5-oxo-4,5-dihydro-2H-pyrazolo[4,3-b]pyridin-2-yl)acetonitrile

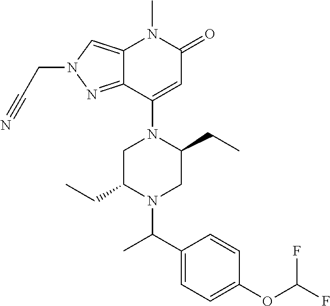 [SMILES: CC[C@H]1CN(C(C)c2ccc(OC(F)F)cc2)[C@H](CC)CN1c1cc(=O)n(C)c2cn(CC#N)nc12]

To a solution of 1-(4-(difluoromethoxy)phenyl)ethan-1-ol (39 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-TLC (DCM/MeOH=20/1) and Prep-HPLC (Method A) to give the titled Compound A19a (7 mg) and Compound A19b (6 mg).

Compound A19a (the earlier peak): ¹H NMR (500 MHz, CD₃OD) δ 7.91 (s, 1H), 7.40 (d, J=8.5 Hz, 2H), 7.12 (d, J=8.5 Hz, 2H), 6.96-6.64 (m, 1H), 5.55 (s, 1H), 5.46 (s, 2H), 3.79-3.73 (m, 1H), 3.46-3.43 (m, 1H), 3.43 (s, 3H), 3.35-3.31 (m, 1H), 3.28-3.24 (m, 1H), 3.03-2.97 (m, 1H), 2.91-2.82 (m, 1H), 2,46-2.35 (m, 1H), 2.16-2.05 (m, 1H), 1.87-1.76 (m, 1H), 1.59-1.46 (m, 2H), 1.33 (d, J=6.5 Hz, 3H), 0.96 (t, J=7.5 Hz, 3H), 0.69 (t, J=7.5 Hz, 3H) ppm. MS: We 499 (M+1)⁺.

Compound A19b (the later peak): ¹H NMR (500 MHz, CD₃OD) δ 7.92 (s, 1H), 7.43 (d, J=8.5 Hz, 2H), 7.09 (d, J=8.5 Hz, 2H), 6.96-6.64 (m, 1H), 5.55 (s, 1H), 5.46 (s, 2H), 3.65-3.57 (m, 1H), 3.54-3.45 (m, 1H), 3.43 (s, 3H), 3.35-3.31 (m, 2H), 3.20-3.10 (m, 1H), 2.66-2.60 (m, 1H), 2.39-2.32 (m, 1H), 2.02-1.89 (m, 1H), 1.71-1.50 (m, 3H), 1.30 (d, 0.1=6.5 Hz, 3H), 1.02 (t, J=7.5 Hz, 3H), 0.63 (t, J=7.5 Hz, 3H) ppm. MS: M/e 499 (M+1)”.

### Compound A20: 2-(7-((2S,5R)-2,5-diethyl-4-(1-(3-fluoro-4-methoxyphenyl)ethyl)piperazin-1-yl)-4-methyl-5-oxo-4,5-dihydro-2H-pyrazolo[4,3-b]pyridin-2-yl)acetonitrile

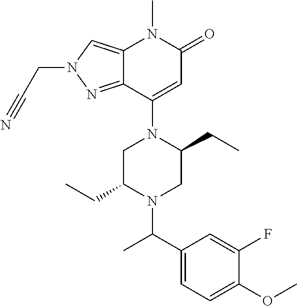 [SMILES: CC[C@H]1CN(C(C)c2ccc(OC)c(F)c2)[C@H](CC)CN1c1cc(=O)n(C)c2cn(CC#N)nc12]

### Step A: 1-(3-fluoro-4-methoxyphenyl)ethan-1-one

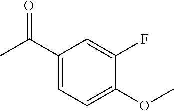

To a solution of 1-(3-fluoro-4-hydroxyphenyl)ethan-1-one (616 mg, 4 mmol) and CH₃I (705 mg, 5 mmol) in CH₃CN (15 mL) was added K₂CO₃ (1.1 g, 8 mmol). The reaction mixture was stirred at 90° C. overnight. The mixture was cooled to room temperature, diluted with water, extracted with EA (80 mL×2), washed with brine, dried over Na₂SO₄, filtered, and concentrated to dryness. The resulting residue was purified by combi flash to give the titled compound (560 mg, 82%). MS: M/e 169 (M+1)⁺.

### Step B: 1-(3-fluoro-4-methoxyphenyl)ethan-1-ol

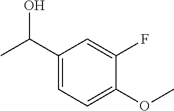

To a solution of 1-(3-fluoro-4-methoxyphenyl)ethan-1-one (560 mg, 3.33 mmol) in MeOH (10 mL) was added NaBH₄ (127 mg, 3.33 mmol) slowly at 0° C. The resulting mixture was stirred at room temperature for 2 hours. The reaction mixture was quenched with H₂O (10 mL) and extracted with EA (80 mL), washed with brine, dried over Na₂SO₄, filtered, and concentrated to dryness to give the desired product (500 mg, 89%). MS: M/e 153 (M-H₂O+1)⁺.

### Step C: 2-(7-((2S,5R)-2,5-diethyl-4-(1-(3-fluoro-4-methoxyphenyl)ethyl)piperazin-1-yl)-4-methyl-5-oxo-4,5-dihydro-2H-pyrazolo[4,3-b]pyridin-2-yl)acetonitrile

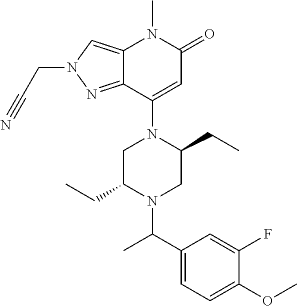 [SMILES: CC[C@H]1CN(C(C)c2ccc(OC)c(F)c2)[C@H](CC)CN1c1cc(=O)n(C)c2cn(CC#N)nc12]

To a solution of 1-(3-fluoro-4-methoxyphenyl)ethan-1-ol (34 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (75 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-TLC (DCM/MeOH=20/1) to give the titled compound (8 mg, 16%). ¹H NMR (500 MHz, CD₃OD) δ 7.93-7.91 (m, 1H), 7.21-6.95 (m, 3H), 5.55 (s, 1H), 5.49-5.44 (m, 2H), 3.94-3.77 (m, 3.5H), 3.75-3.66 (m, 0.5H),3.59-3.41 (m, 1.5H), 3.43 (s, 3H), 3.35-3.31 (m, 1H), 3.28-3.25 (m, 0.5H), 3.18-3.08 (m, 0.5H), 3.02-2.96 (m, 0.5H), 2.89-2.81 (m, 0.5H), 2.66-2,58 (m, 0.5H), 2,49-2.35 (m, 1H), 2.16-1.46 (m, 4H), 1.36-1,24 (m, 3H), 1.05-0.91 (m, 3H), 0.76-0.59 (m, 3H) ppm. MS: M/e 481 (M+1)⁺.

### Compound A21: 2-(7-((2S,5R)-2,5-diethyl-4-(1-(3-methyl-4-(oxetan-3-yloxy)phenyl)ethyl)piperazin-1-yl)-4-methyl-5-oxo-4,5-dihydro-2H-pyrazolo[4,3-b]pyridin-2-yl)acetonitrile

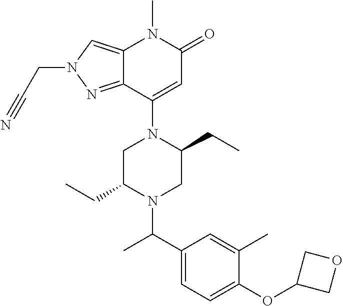 [SMILES: CC[C@H]1CN(C(C)c2ccc(OC3COC3)c(C)c2)[C@H](CC)CN1c1cc(=O)n(C)c2cn(CC#N)nc12]

### Step A: 1-(3-methyl-4-(oxetan-3-yloxy)phenyl)ethan-1-one

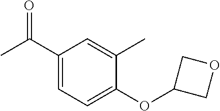

To a solution of 1-(4-hydroxy-3-methylphenyl)ethan-1-one (500 mg, 3.33 mmol) in DMF (3 mL) was added oxetan-3-yl 4-methylbenzenesulfonate (760 mg, 3.33 mmol), TBAB (107 mg, 0.33 mmol) and Cs₂CO₃ (2.17 g, 6.67 mmol). The resulting mixture was stirred at 80° C. overnight. The reaction mixture was diluted with DCM (200 mL). The organic layer was washed with water, dried over Na₂SO₄ and concentrated to give the titled compound (470 mg, 68%). MS: M/e 207 (M+1)⁺.

### Step B: 1-(3-methyl-4-(oxetan-3-yloxy)phenyl)ethan-1-ol

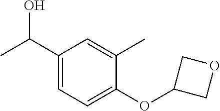

To a solution of 1-(3-methyl-4-(oxetan-3-yloxy)phenyl)ethan-1-one (470 mg, 2.28 mmol) in MeOH (15 mL) was added NaBH₄ (173 mg, 4.56 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (400 mg, 84%). MS: M/e 209 (M+1)⁺.

### Step C: 2-(7-((2S,5R)-2,5-diethyl-4-(1-(3-methyl-4-(oxetan-3-yloxy)phenyl)ethyl)piperazin-1-yl)-4-methyl-5-oxo-4,5-dihydro-2H-pyrazolo[4,3-b]pyridin-2-yl)acetonitrile

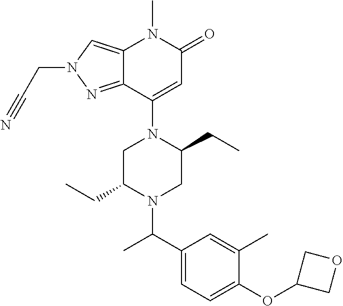 [SMILES: CC[C@H]1CN(C(C)c2ccc(OC3COC3)c(C)c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(3-methyl-4-(oxetan-3-yloxy)phenyl)ethan-1-ol (32 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled Compound A21a (20 mg) and Compound A21b (15 mg).

Compound A21a (the earlier peak): ¹H NMR (400 MHz, CD₃OD) δ 7.92 (s, 1H), 7.22 (d, J=11.5 Hz, 1H), 7.16-7.07 (m, 1H), 6.46 (dd, J=18.8, 8.3 Hz, 1H), 5.55 (d, J=3.1 Hz, 1H), 5.47 (d, J=1.6 Hz, 2H), 5.31-5.24 (m, 1H), 5.03 (dd, J=12.6, 6.3 Hz, 2H), 4.72-4.67 (m, 2H), 3.91-3.60 (m, J=113.1 Hz, 1H), 3.59-3.43 (m, 1H), 3.43 (s, 3H), 3.33-3.31 (m, J=1.6 Hz, 1H), 3.05 (d, J=3.3 Hz, 2H), 2.65 (s, 1H), 2.65-2,42 (m, 1H), 2.26 (d, J=11.0 Hz, 3H), 2.06-1.49 (m, 4H), 1.37 (dd, J=39.9, 6.5 Hz, 3H), 0.99 (dt, J=25.0, 7.4 Hz, 3H), 0.68 (dt, J=36.6, 7.3 Hz, 3H) ppm. MS: M/e 519 (M+1)⁺.

Compound A21b (the later peak): ¹H NMR (400 MHz, CD₃OD) δ 7.92 (s, 1H), 7.14-6.99 (m, 2H), 6.35 (dd, J=8.4, 4.2 Hz, 1H), 5.48-5.43 (m, 1H), 5.24-5.17 (m, 1H), 5.10-4.96 (m, 3H), 4.71-4.61 (m, 2H), 4.22-3.91 (m, 1H), 3.48 (s, 3H), 3.44-3.30 (m, 3H), 3.29-3.13 (m, 1H), 3.06-2.63 (m, 3H), 2.19 (s, 3H), 1.67 (dd, J=16.8, 7.3 Hz, 3H), 1.62-1.07 (m, 4H), 0.90-0.76 (m, 6H) ppm. MS: M/e 519 (M+1)⁺.

### Compound A22: 2-(7-((2S,5R)-2,5-diethyl-4-(1-(2-fluoro-4-(oxetan-3-yloxy)phenyl)ethyl)piperazin-1-yl)-4-methyl-5-oxo-4,5-dihydro-2H-pyrazolo[4,3-b]pyridin-2-yl)acetonitrile

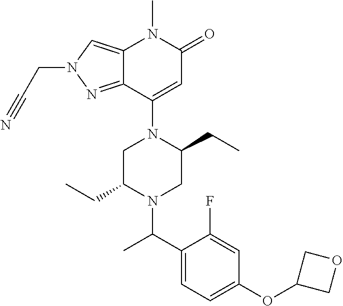 [SMILES: CC[C@H]1CN(C(C)c2ccc(OC3COC3)cc2F)[C@H](CC)CN1c1cc(=O)n(C)c2cn(CC#N)nc12]

### Step A: 1-(2-fluoro-4-(oxetan-3-yloxy)phenyl)ethan-1-one

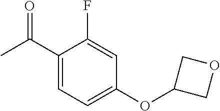

To a solution of 1-(2-fluoro-4-hydroxyphenyl)ethan-1-one (500 mg, 3.25 mmol) in DMF (3 mL) was added oxetan-3-yl 4-methylbenzenesulfonate (740 mg, 3.24 mmol), TBAB (107 mg, 0.32 mmol) and Cs₂CO₃ (2.12 g, 6.49 mmol). The resulting mixture was stirred at 80° C. overnight. The reaction mixture was diluted with DCM (200 mL). The organic layer was washed with water, dried over Na₂SO₄ and concentrated to give the titled compound (500 mg, 73%). MS: M/e 211 (M+1)⁺.

### Step B: 1-(2-fluoro-4-(oxetan-3-yloxy)phenyl)ethan-1-ol

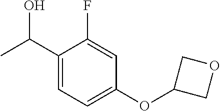

To a solution of 1-(2-fluoro-4-(oxetan-3-yloxy)phenyl)ethan-1-one (500 mg, 2.38 mmol) in MeOH (15 mL) was added NaBH₄ (181 mg, 4.76 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (460 mg, 91%). MS: M/e 213 (M+1)⁺.

### Step C: 2-(7-((2S,5R)-2,5-diethyl-4-(1-(2-fluoro-4-(oxetan-3-yloxy)phenyl)ethyl)piperazin-1-yl)-4-methyl-5-oxo-4,5-dihydro-2H-pyrazolo[4,3-b]pyridin-2-yl)acetonitrile

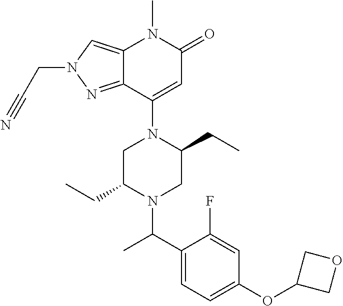 [SMILES: CC[C@H]1CN(C(C)c2ccc(OC3COC3)cc2F)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(2-fluoro-4-(oxetan-3-yloxy)phenyl)ethan-1-ol (32 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled compound(30 mg, 38%). ¹H NMR (400 MHz, CD₃OD) δ 7.92 (d, J=2.9 Hz, 1H), 7.46 (dt, J=37.5, 8.5 Hz, 1H), 6.66-6.58 (m, 1H), 6.52 (ddd, J=14.5, 11.9, 2,5 Hz, 1H), 5.54 (s, 1H), 5.46 (d, J=3.9 Hz, 2H), 5.27 (dt, J=10.9, 5.4 Hz, 1H), 5.01 (dd, J=11.5, 6.4 Hz, 2H), 4.69-4.64 (m, 2H), 4.11-3.93 (m, 1H), 3.58-3.44 (m, 1H), 3.43 (s, 3H), 3.30-3.21 (m, 2H), 3.18-2.63 (m, 2H), 2,49-2.30 (m, 1H), 2.10-1.88 (m, 1H), 1.86-1.65 (m, 1H), 1.65-1.52 (m, 1H), 1.52-1.41 (m, 1H), 1.32 (dd, J=21.8, 6.6 Hz, 3H), 0.98 (dt, J=35.0, 7.4 Hz, 3H), 0.69 (dt, J=46.0, 7.4 Hz, 3H). MS: M/e 523 (M+1)⁺.

### Compound A23: 2-(7-((2S,5R)-2,5-diethyl-4-(1-(3-fluoro-4-(oxetan-3-yloxy)phenyl)ethyl)piperazin-1-yl)-4-methyl-5-oxo-4,5-dihydro-2H-pyrazolo[4,3-b]pyridin-2-yl)acetonitrile

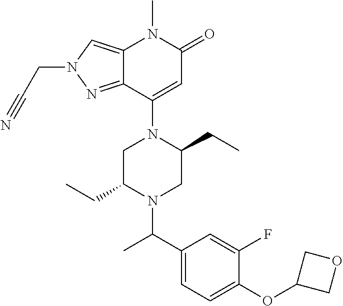 [SMILES: CC[C@H]1CN(C(C)c2ccc(OC3COC3)c(F)c2)[C@H](CC)CN1c1cc(=O)n(C)c2cn(CC#N)nc12]

### Step A: 1-(3-fluoro-4-(oxetan-3-yloxy)phenyl)ethan-1-one

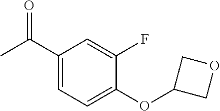

To a solution of 1-(3-fluoro-4-hydroxyphenyl)ethan-1-one (500 mg, 3.25 mmol) in DMF (3 mL) was added oxetan-3-yl 4-methylbenzenesulfonate (740 mg, 3.24 mmol), TBAB (107 mg, 0.32 mmol) and Cs₂CO₃ (2.12 g, 6.49 mmol). The resulting mixture was stirred at 80° C. overnight. The reaction mixture was diluted with DCM (200 mL). The organic layer was washed with water, dried over Na₂SO₄ and concentrated to give the titled compound (500 mg, 73%). MS: M/e 211 (M+1)⁺.

### Step B: 1-(3-fluoro-4-(oxetan-3-yloxy)phenyl)ethan-1-ol

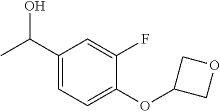

To a solution of 1-(3-fluoro-4-(oxetan-3-yloxy)phenyl)ethan-1-one (500 mg, 2.38 mmol) in MeOH (15 mL) was added NaBH₄ (181 mg, 4.76 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (460 mg, 91%). MS: M/e 213 (M+1)⁺.

### Step C: 2-(7-((2S,5R)-2,5-diethyl-4-(1-(3-fluoro-4-(oxetan-3-yloxy)phenyl)ethyl)piperazin-1-yl)-4-methyl-5-oxo-4,5-dihydro-2H-pyrazolo[4,3-b]pyridin-2-yl)acetonitrile

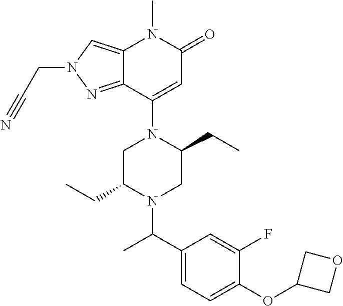 [SMILES: CC[C@H]1CN(C(C)c2ccc(OC3COC3)c(F)c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(3-fluoro-4-(oxetan-3-yloxy)phenyl)ethan-1-ol (32 mg, 0 15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled Compound A23a (17 mg) and Compound A23b (15 mg).

Compound A23a (the earlier peak): ¹H NMR (400 MHz, CD₃OD) δ 7.92 (s, 1H), 7.21 (dd, J=12,5, 1.9 Hz, 1H), 7.07 (d, J=8.4 Hz, 1H), 6.71 (t, J=8.4 Hz, 1H), 5.55 (s, 1H), 5.47 (s, 2H), 5.31-5.25 (m, 1H), 4.99 (t, J=6.7 Hz, 2H), 4.84-4.78 (m, 2H), 4.74-4.70 (m, 2H), 3.55 (q, J=6.6 Hz, 1H), 3.48 (d, J=11.8 Hz, 1H), 3.43 (s, 3H), 3.11 (d, J=10.0 Hz, 1H), 2.62 (dd, J=12,4, 3.7 Hz, 1H), 2.36 (d, J=12,4 Hz, 1H), 2.03-1.86 (m, 1H), 1.69-1.59 (m, 2H), 1.56-1.48 (m, 1H), 1,27 (d, J=6.5 Hz, 3H), 1.01 (t, J=7.4 Hz, 3H), 0.65 (t, J=7.4 Hz, 3H) ppm. MS: M/e 523 (M+1)”.

Compound A23b (the later peak): ¹H NMR (400 MHz, CD₃OD) δ 7.91 (s, 1H), 7.17 (dd, J=12,4, 1.9 Hz, 1H), 7.06 (d, J=8.1 Hz, 1H), 6.73 (t, J=8.5 Hz, 1H), 5.55 (s, 1H), 5.46 (s, 2H), 5.32-5.27 (m, 1H), 5.00 (t, J=6.8 Hz, 2H), 4.74 (t, J=5.6 Hz, 2H), 3.70 (q, J=6.3 Hz, 1H), 3.43 (s, 3H), 3.30-3.25 (m, 3H), 2.97 (d, J=10.2 Hz, 1H), 2.85 (dd, J=12.0, 3.9 Hz, 1H), 2,42 (s, 1H), 2.15-2.03 (m, 1H), 1.86-1.76 (m, 1H), 1.55-1.46 (m, 2H), 1.30 (d, J=6.5 Hz, 3H), 0.95 (t, J=7.4 Hz, 3H), 0.71 (t, J=7.4 Hz, 3H) ppm. MS: M/e 523 (M+1)⁺.

### Compound A24: 2-(7-((2S,5R)-2,5-diethyl-4-(1-(4-((3-methyloxetan-3-yl)oxy)phenyl)ethyl)piperazin-1-yl)-4-methyl-5-oxo-4,5-dihydro-2H-pyrazolo[4,3-b]pyridin-2-yl)acetonitrile

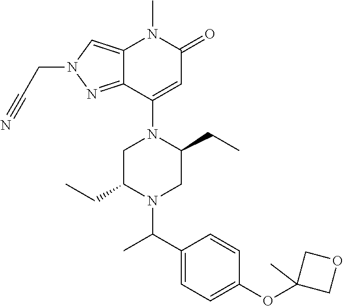 [SMILES: CC[C@H]1CN(C(C)c2ccc(OC3(C)COC3)cc2)[C@H](CC)CN1c1cc(=O)n(C)c2cn(CC#N)nc12]

### Step A: 4-((3-methyloxetan-3-yl)oxy)benzonitrile

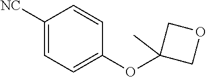

To a solution of 4-fluorobenzonitrile (200 mg, 1.65 mmol) in DMF (3 mL) was added 3-methyloxetan-3-ol (291 mg, 3.31 mmol), TBAB (53 mg, 0.16 mmol) and potassium t-butoxide (370 g, 3.31 mmol). The resulting mixture was stirred at RT for 5 hrs. The reaction mixture was diluted with DCM (200 mL). The organic layer was washed with water, dried over Na₂SO₄ and concentrated to give the titled compound (275 mg, 88%). MS: M/e 190 (M+1)⁺.

### Step B: 1-(4-((3-methyloxetan-3-yl)oxy)phenyl)ethan-1-one

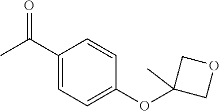

To a solution of 4-((3-methyloxetan-3-yl)oxy)benzonitrile (275 mg, 1.45 mmol) in THF (15 mL) was added methylmagnesium bromide (2.2 mL, 2.18 mmol) at −78° C. and the resulting mixture was stirred at RT for 2 mins. The mixture was added con·HCl (1 mL) and stirred for 1 h. The reaction mixture was diluted with DCM (200 mL). The organic layer was washed with water, dried over Na₂SO₄ and concentrated to give the titled compound (200 mg, 67%). MS: M/e 207 (M+1)⁺.

### Step C: 1-(4-((3-methyloxetan-3-yl)oxy)phenyl)ethan-1-ol

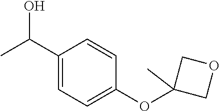

To a solution of 1-(4-((3-methyloxetan-3-yl)oxy)phenyl)ethan-1-one (200 mg, 0.97 mmol) in MeOH (15 mL) was added NaBH₄ (55 mg, 1.45 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (180 mg, 89%). MS: M/e 209 (M+1)⁺.

### Step D: 2-(7-((2S,5R₁-2,5-diethyl-4-(1-(4-((3-methyloxetan-3-yl)oxy)phenyl)ethyl)piperazin-1-yl)-4-methyl-5-oxo-4,5-dihydro-2H-pyrazolo[4,3-b]pyridin-2-yl)acetonitrile

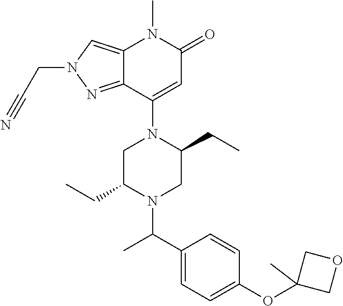 [SMILES: CC[C@H]1CN(C(C)c2ccc(OC3(C)COC3)cc2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(4-((3-methyloxetan-3-yl)oxy)phenyl)ethan-1-ol (32 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled compound(30 mg, 38%). ¹H NMR (400 MHz, CD₃OD) δ 7.91 (d, J=2.6 Hz, 1H), 7.28 (dd, J=14.5, 8.6 Hz, 2H), 6.65 (dd, J=10.2, 8.7 Hz, 2H), 5.54 (d, J=3.3 Hz, 1H), 5.46 (d, J=3.7 Hz, 2H), 4.89 (d, J=7.0 Hz, 2H), 4.87 (s, 1H), 4.60 (dd, J=8.7, 4.8 Hz, 2H), 3.73-3.50 (m, 1H), 3.48 (s, 1H), 3.43 (d, J=0.7 Hz, 3H), 3.17 (dd, J=42.3, 27.6 Hz, 1H), 3.03-2.80 (m, 1H), 2.62-2,42 (m, 1H), 2.39-2.03 (m, 1H), 2.00-1.76 (m, 1H), 1.70 (d, J=8.9 Hz, 3H), 1.68-1.41 (m, 3H), 1.30 (dd, J=14.8, 6.5 Hz, 3H), 0.98 (dt, J=31.5, 7.4 Hz, 3H), 0.65 (dt, J=35.5, 7.3 Hz, 3H) ppm. MS: M/e 519 (M+1)⁺.

### Compound A25: 2-(7-((2S,5R)-4-(1-(4-((dimethylamino)methyl)-3,5-difluorophenyl)ethyl)-2,5-diethylpiperazin-1-yl)-4-methyl-5-oxo-4,5-dihydro-2H-pyrazolo[4,3-bipyridin-2-yl)acetonitrile

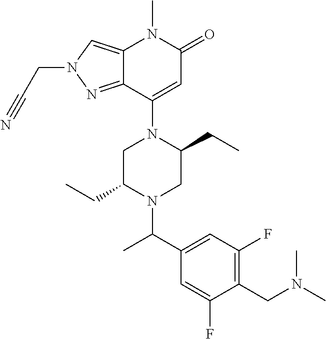 [SMILES: CC[C@H]1CN(C(C)c2cc(F)c(CN(C)C)c(F)c2)[C@H](CC)CN1c1cc(=O)n(C)c2cn(CC#N)nc12]

### Step A: 1-(4-bromo-2,6-difluorophenyl)-N,N-dimethylmethanamine

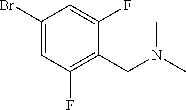

A solution of 4-bromo-2,6-difluorobenzaldehyde (500 mg, 2.26 mmol), dimethylamine hydrochloride (223 mg, 2.72 mmol) and STAB (719 mg, 3.39 mmol) in DCM (10 ml) was stirred at RT overnight. The solution was diluted with DCM (10 ml), washed with brine (10 ml), dried over Na₂SO₄ and concentrated to dryness. The resulting residue was purified by flash column chromatography with 0-20% EtOAc in PE to give the titled compound (510 mg, 90%). MS: M/e 250 (M+1)⁺.

### Step B: 1-(4-((dimethylamino)methyl)-3.5-difluorophenyl)ethan-1-one

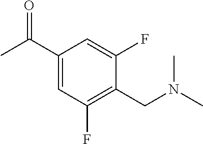

A solution of 1-(4-bromo-2,6-difluorophenyl)-N,N-dimethylmethanamine (510 mg, 2.04 mmol), ethyl tributylstannanecarboxylate (921 mg, 2,55 mmol) and Pd(PPh₃)₂Cl₂ (71.6 mg, 0.10 mmol) in toluene(10 ml) was stirred at 100° C. overnight. The solution was cooled to RT. HCl/1,4-dioxane (4M, 2 ml) was added and stirred at RT for 10 min. The solution was diluted with EtOAc (20 ml), washed with aq. Na₂CO₃ (10 ml×2), dried over Na₂SO₄ and concentrated to dryness. The resulting residue was purified by flash column chromatography with 15-40% EA in PE to give the titled compound (283 mg, 65%). MS: M/e 214 (M+1)⁺.

### Step C: 1-(4-((dimethylamino)methyl)-3.5-difluorophenyl)ethan-1-ol

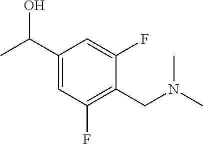

A solution of 1-(4-((dimethylamino)methyl)-3,5-difluorophenyl)ethan-1-one (283 mg, 1.33 mmol) and NaBH₄ (50.5 mg, 1.33 mmol) in MeOH (4 ml) was stirred at RT for 15 min. The solution was diluted with EtOAc (15 ml), washed with brine (10 ml×2), dried over Na₂SO₄ and concentrated to dryness to give the titled compound (268 mg, 93%), which was used directly for the next step without further purification. MS: M/e 216 (M+1)⁺.

### Step D: 2-(7-((2S,5R)-4-(1-(4-((dimethylamino)methyl)-3.5-difluorophenyl)ethyl-2,5-diethylpiperazin-1-yl)-4-methyl-5-oxo-4,5-dihydro-2H-pyrazolo[4,3-b]pyridin-2-yl)acetonitrile

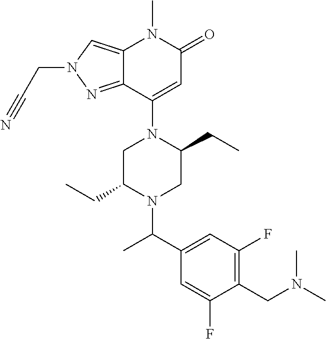 [SMILES: CC[C@H]1CN(C(C)c2cc(F)c(CN(C)C)c(F)c2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol), 1-(4-((dimethylamino)methyl)-3,5-difluorophenyl)ethan-1-ol (49.2 mg, 0.23 mmol), (cyanomethyl)trimethylphosphonium iodide (111 mg, 0.46 mmol) and DIPEA (197 mg, 1.53 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A25, which was further separated into Compound A25a (8.5 mg) and Compound A25b (9.3 mg) by Prep-HPLC (Method A).

Compound A25a (the earlier peak): ¹H NMR (500 MHz, DMSO-d₆) δ 8.22 (s, 1H), 7.98 (s, 1H), 7.09 (d, J=8.3 Hz, 2H), 5.61 (s, 2H), 5.40 (s, 1H), 3.77 (q, J=6.5 Hz, 1H), 3.46 (s, 2H), 3.35 (s, 2H), 3.27 (s, 3H), 3.18 (d, J=6.5 Hz, 1H), 2.85 (d, J=6.5 Hz, 1H), 2.76 (d, J=6.5 Hz, 1H), 2.33 (s, 1H), 2.14 (s, 6H), 2.05-1.94 (m, 1H), 1.68-1.57 (m, 1H), 1.50-1.41 (m, 2H), 1,27 (d, J=6.4 Hz, 3H), 0.85 (t, J=7.1 Hz, 3H), 0.66 (t, J=7.3 Hz, 3H) ppm. MS: M/e 526 (M+1)⁺.

Compound A25b (the later peak): ¹H NMR (500 MHz, DMSO-d₆) δ 8.27 (s, 1H), 7.98 (s, 1H), 7.10 (d, J=8.4 Hz, 2H), 5.61 (s, 2H), 5.39 (s, 1H), 3.61 (q, J=6.2 Hz, 1H), 3.47 (q, J=8.9 Hz, 2H), 3.35 (d, J=11.0 Hz, 2H), 3.27 (s, 3H), 3.02 (d, J=9.9 Hz, 1H), 2,54 (d, J=9.9 Hz, 2H), 2.21 (d, J=12,4 Hz, 1H), 2.11 (s, 6H), 1.96-1.84 (m, 1H), 1.64-1.53 (m, 1H), 1.50-1.35 (m, 2H), 1,24 (d, J=6.4 Hz, 3H), 0.94 (t, J=7.1 Hz, 3H), 0.56 (t, J=7.0 Hz, 3H) ppm. MS: M/e 526 (M+1)⁺.

### Compound A26: 2-(7-((2S,5R)-2,5-diethyl-4-(1-(4-(pyrrolidin-1-ylmethyl)phenyl)ethyl)piperazin-1-yl)-4-methyl-5-oxo-4,5-dihydro-2H-pyrazolo[4,3-b]pyridin-2-yl)acetonitrile

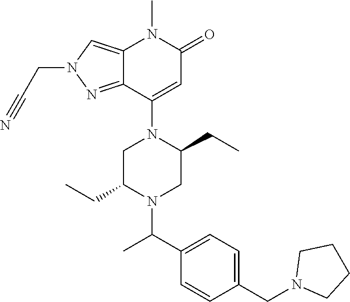 [SMILES: CC[C@H]1CN(C(C)c2ccc(CN3CCCC3)cc2)[C@H](CC)CN1c1cc(=O)n(C)c2cn(CC#N)nc12]

### Step A: 1-(4-bromobenzyl)pyrrolidine

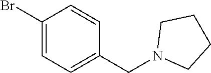 [SMILES: Brc1ccc(CN2CCCC2)cc1]

A solution of 4-bromobenzaldehyde (500 mg, 2.70 mmol), pyrrolidine (230 mg, 3.24 mmol) and STAB (859 mg, 4.05 mmol) in DCM (10 ml) was stirred at RT for 5 hrs. The solution was diluted with DCM (10 ml), washed with brine (10 ml), dried over Na₂SO₄ and concentrated to dryness. The resulting residue was purified by flash column chromatography with 0-10/0 MeOH in DCM to give the titled compound (630 mg, 97%). MS: M/e 240,242 (M+1)⁺.

### Step B: 1-(4-(pyrrolidin-1-ylmethyl)phenyl)ethan-1-one

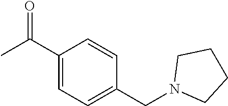

A solution of 1-(4-bromobenzyl)pyrrolidine (630 mg, 2.63 mmol), ethyl tributylstannanecarboxylate (1.42 g, 3.93 mmol) and Pd(PPh₃)₂Cl₂ (92 mg, 0.13 mmol) in toluene(10 ml) was stirred at 100° C. overnight. The solution was cooled to RT. HCl/1,4-dioxane (4M, 2 ml) was added and stirred at RT for 10 min. The solution was diluted with EtOAc (20 ml), washed with aq. Na₂CO₃ (10 ml×2), dried over Na₂SO₄ and concentrated to dryness. The resulting residue was purified by flash column chromatography with 0-10% MeOH in DCM to give the titled compound (316 mg, 59%). MS: M/e 204 (M+1)⁺.

### Step C: 1-(4-(pyrrolidin-1-ylmethyl)phenyl)ethan-1-ol

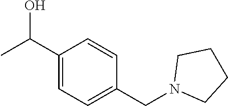

A solution of 1-(4-(pyrrolidin-1-ylmethyl)phenyl)ethan-1-one (316 mg, 1.56 mmol) and NaBH₄ (59.2 mg, 1.56 mmol) in MeOH (5 ml) was stirred at RT for 15 min. The solution was diluted with EtOAc (15 ml), washed with brine (10 ml×2), dried over Na₂SO₄ and concentrated to dryness to give the titled compound (300 mg, 94%), which was used directly for the next step without further purification. MS: M/e 206 (M+1)⁺.

### Step D: 2-(7-((2S,5R)-2,5-diethyl-4-(1-(4-(pyrrolidin-1-ylmethyl)phenyl)ethyl)piperazin-1-yl)-4-methyl-5-oxo-4,5-dihydro-2H-pyrazolo[4,3-b]pyridin-2-yl)acetonitrile

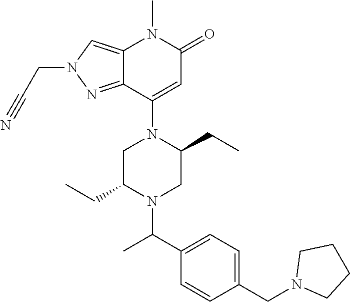 [SMILES: CC[C@H]1CN(C(C)c2ccc(CN3CCCC3)cc2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol), 1-(4-(pyrrolidin-1-ylmethyl)phenyl)ethan-1-ol (62,5 mg, 0.30 mmol), (cyanomethyl)trimethylphosphonium iodide (111 mg, 0.46 mmol) and DIPEA (197 mg, 1.53 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A26, which was further separated into Compound A26a (11 mg) and Compound A26b (1 mg) by Prep-HPLC (Method A).

Compound A26a (the earlier peak): ¹H NMR (500 MHz, DMSO-4) δ 8.22 (s, 1.4H), 7.97 (s, 1H), 7.27 (d, J=1.5 Hz, 4H), 5.60 (s, 2H), 5.38 (s, 1H), 3.67 (q, J=6.5 Hz, 1H), 3.57 (s, 2H), 3.33 (s, 1H), 3.27 (s, 4H), 3.10 (d, J=12.0 Hz, 1H), 2.90 (d, J=11.5 Hz, 1H), 2.74 (d, J=8.4 Hz, 1H), 2,43 (s, 4H), 2.31 (s, 1H), 2.07-1.96 (m, 1H), 1.73-1.67 (m, 4H), 1.65-1.57 (m, 1H), 1.48-1.38 (m, 2H), 1,27 (d, J=6.4 Hz, 3H), 0.86 (t, J=7.1 Hz, 3H), 0.58 (t, J=7.3 Hz, 3H) ppm. MS: M/e 516 (M+1)⁺.

Compound A26b (the later peak): ¹H NMR (500 MHz, DMSO-4) 6 8.27 (s, 1H), 7.97 (s, 1H), 7.30 (d, J=8.0 Hz, 2H), 7.24 (d, J=8.0 Hz, 2H), 5.61 (s, 2H), 5.38 (s, 1H), 3.54 (s, 3H), 3.27 (s, 7H), 3.04 (d, J=10.4 Hz, 1H), 2,40 (s, 4H), 2.24 (d, J=12.1 Hz, 1H), 1.91-1.81 (m, 1H), 1.70-1.65 (m, 4H), 1.60-1.40 (m, 3H), 1,24 (d, J=6.4 Hz, 3H), 0.94 (t, J=7.4 Hz, 3H), 0.50 (t, J=7.1 Hz, 3H) ppm. MS: M/e 516 (M+1)⁺.

### Compound A27: 2-(7-((2S,5R)-4-(1-(4-((3,3-difluoroazetidin-1-yl)methyl)phenyl)ethyl)-2,5-diethylpiperazin-1-yl)-4-methyl-5-oxo-4,5-dihydro-2H-pyrazolo[4,3-b]pyridin-2-yl)acetonitrile

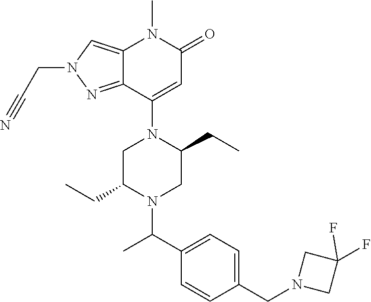 [SMILES: CC[C@H]1CN(C(C)c2ccc(CN3CC(F)(F)C3)cc2)[C@H](CC)CN1c1cc(=O)n(C)c2cn(CC#N)nc12]

### Step A: 1-(4-((3.3-difluoroazetidin-1-yl)methyl)phenyl)ethan-1-one

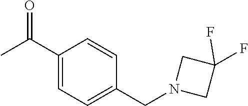 [SMILES: CC(=O)c1ccc(CN2CC(F)(F)C2)cc1]

A solution of 1-(4-(bromomethyl)phenyl)ethan-1-one (500 mg, 2.36 mmol), 3,3-difluoroazetidine hydrochloride (307 mg, 2.36 mmol) and DIPEA (913 mg, 7.08 mmol) in THF (6 ml) was stirred at RT for 6 hrs. The solution was diluted with EA (10 ml), washed with brine (10 ml), dried over Na₂SO₄ and concentrated to dryness. The resulting residue was purified by flash column chromatography with 0-20% EA in PE to give the titled compound (380 mg, 72%). MS: M/e 226 (M+1)⁺.

### Step B: 144-((33-difluoroazetidin-1-yl)methyl)phenylethan-1-ol

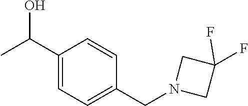 [SMILES: CC(O)c1ccc(CN2CC(F)(F)C2)cc1]

A solution of 1-(4-((3,3-difluoroazetidin-1-yl)methyl)phenyl)ethan-1-one (380 mg, 1.69 mmol) and NaBH₄ (64 mg, 1.68 mmol) in MeOH (5 ml) was stirred at RT for 15 min. The solution was diluted with EtOAc (15 ml), washed with brine (10 ml×2), dried over Na₂SO₄ and concentrated to dryness to give the titled compound (380 mg, 99%), which was used directly for the next step without further purification. MS: We 228 (M+1)⁺.

### Step C: 2-(7-((2S,5R)-4-(-(4-((3.3-difluoroazetidin-1-yl)methyl)phenyl)ethyl)-2,5-diethylpiperazin-1-yl-4-methyl-5-oxo-4,5-dihydro-2H-pyrazolo[4,3-b]pyridin-2-yl)acetonitrile

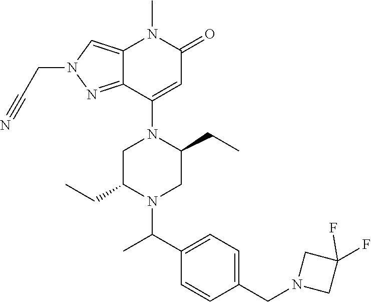 [SMILES: CC[C@H]1CN(C(C)c2ccc(CN3CC(F)(F)C3)cc2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol), 1-(4-((3,3-difluoroazetidin-1-yl)methyl)phenyl)ethan-1-ol (104 mg, 0.46 mmol), (cyanomethyl)trimethylphosphonium iodide (111 mg, 0.46 mmol) and DIPEA (197 mg, 1.53 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography and then Prep-HPLC (Method A) to give the titled compound (19 mg). ¹H NMR (500 MHz, DMSO-d₆) δ 8.36 (s, 0.15H), 7.97 (d, J=2.1 Hz, 1H), 7.34-7.22 (m, 4H), 5.61 (s, 2H), 5.38 (s, 1H), 3.72-3.66 (m, 2,5H), 3.57 (q, .=12.6 Hz, 4.5H), 3.34 (s, 3H), 3.27 (s, 3H), 3.07 (dd, J=30.2, 10.0 Hz, 1H), 2.90 (d, J=11.4 Hz, 0.5H), 2.74 (d, J=8.4 Hz, 0.5H), 2.30 (s, 0.5H), 2.22 (d, J=11.7 Hz, 0.5H), 2.06-1.81 (m, 1H), 1.66-1.51 (m, 1H), 1.50-1.39 (m, 2H), 1,25 (dd, J=16.7, 6.4 Hz, 3H), 0.90 (dt, J=41.8, 7.3 Hz, 3H), 0.57 (dt, J=41.8, 7.5 Hz, 3H) ppm. MS: M/e 538 (M+1)⁺.

### Compound A28: 2-(7-((2S,5R)-4-(1-(4-((dimethylamino)methyl)phenyl)ethyl)-2,5-diethylpiperazin-1-yl)-4-methyl-5-oxo-4,5-dihydro-2H-pyrazolo[4,3-b]pyridin-2-yl)acetonitrile

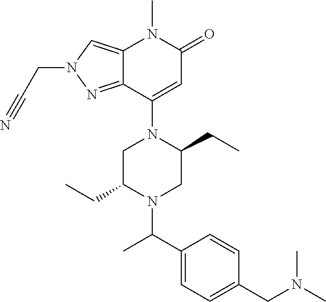 [SMILES: CC[C@H]1CN(C(C)c2ccc(CN(C)C)cc2)[C@H](CC)CN1c1cc(=O)n(C)c2cn(CC#N)nc12]

### Step A: 1-(4-((dimethylamino)methyl)phenyl)ethan-1-one

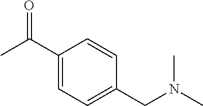

To a stirred solution of 1-(4-(bromomethyl)phenyl)ethan-1-one (213 mg, 1 mmol), dimethylamine hydrochloride (81.5 mg, 1 mmol) in EtOH/CH₂Cl₂ (5 mL/3 mL) was added DIPEA (258 mg, 2 mmol). After the addition, the reaction was stirred for 3 hours. The reaction was poured into H₂O (10 mL) and extracted with CH₂Cl₂ (10 mL×3). The combined organic layers were washed with brine, dried over Na₂SO₄, concentrated to dryness. The resulting residue was purified by flash column chromatography to give the titled compound (80 mg, 45%). MS: M/e 178 (M+1)”.

### Step B: 1-(4-((dimethylamino)methyl)phenyl)ethan-1-ol

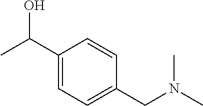

To a stirred solution of 1-(4-((dimethylamino)methyl)phenyl)ethan-1-one (80 mg, 0.45 mmol) in MeOH (5 mL) was added NaBH₄ (17 mg, 0.45 mmol). After then, the mixture was stirred for 20 min. The reaction was quenched with H₂O, concentrated to give the aqueous layer, which was extracted with EtOAc (15 mL×3). The combined organic layers were washed with brine, dried over Na₂SO₄, concentrated to give the titled compound (80 mg, 100%). MS: M/e 180 (M+1)⁺.

### Step C: 2-(7-((2S,5R)-4-(1-(4-((dimethylamino)methyl)phenyl)ethyl)-2,5-diethylpiperazin-1-yl-4-methyl-5-oxo-4,5-dihydro-2H-pyrazolo[4,3-b]pyridin-2-yl)acetonitrile

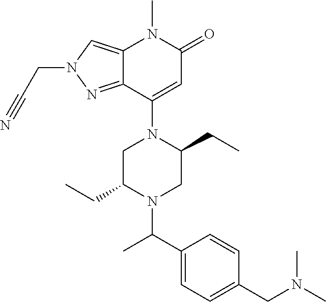 [SMILES: CC[C@H]1CN(C(C)c2ccc(CN(C)C)cc2)[C@H](CC)CN1c1cc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), 1-(4-((dimethylamino)methyl)phenyl)ethan-1-ol (28 mg, 0.15 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄, concentrated to dryness. The resulting residue was purified by Prep-HPLC (Method A) to give the titled compound (2 mg).

¹H NMR (500 MHz, CD₃OD) δ 7.94-7.90 (m, 1H), 7.53-7.37 (m, 4H), 5.57-5.52 (m, 1H), 5.49-5.43 (m, 2H), 3.99 (s, 2H), 3.83-3.61 (m, 1H), 3.53-3.45 (m, 1H), 3.43 (s, 3H), 3.18-2.99 (m, 1H), 2.90-2.73 (m, 1H), 2.69-2,56 (m, 7H), 2,44-2.32 (m, 1H), 2.16-1.77 (m, 2H), 1.73-1.48 (m, 3H), 1.42-1,29 (m, 3H), 1.07-0.93 (m, 3H), 0.70-0.57 (m, 3H) ppm. MS: M/e 490 (M+1)⁺.

### Compound A29: 2-(7-((2S,5R)-2,5-diethyl-4-(1-(4-((methylamino)methyl)phenyl)ethyl)piperazin-1-yl)-4-methyl-5-oxo-4,5-dihydro-2H-pyrazolo[4,3-b]pyridin-2-yl)acetonitrile

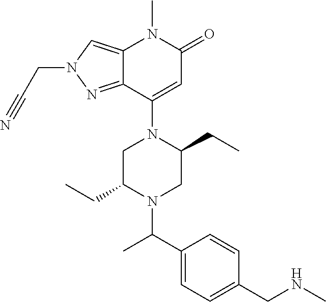 [SMILES: CC[C@H]1CN(C(C)c2ccc(CNC)cc2)[C@H](CC)CN1c1cc(=O)n(C)c2cn(CC#N)nc12]

### Step A: tert-butyl (4-cyanobenzyl)(methyl)carbamate

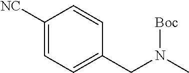 [SMILES: CN(Cc1ccc(C#N)cc1)C(=O)OC(C)(C)C]

To a stirred solution of 4-formylbenzonitrile (1.31 g, 10 mmol) in CH₂Cl₂ (20 mL) was added NH₂Me (2.0 M, 10 mL, 20 mmol), followed by AcOH (0.5 mL). After stirred for an hour, NaBH(OAc)₃ (3.18 g, 15 mmol) was added and the mixture was stirred overnight. H₂O (20 mL) was added and extracted with CH₂Cl₂ (20 mL×2). The combined organic layers were combined with H₂O (10 mL), K₂CO₃ (2.76 g, 20 mmol) was added followed by Boc₂O (3.27 g, 1.5 mmol). Then the mixture was stirred for 2 hours. The organic layer was separated, washed with brine, dried over Na₂SO₄, concentrated to dryness. The resulting residue was purified by flash column chromatography to give the titled compound (1.5 g, 61%). MS: M/e 247 (M+1)⁺.

### Step B: tert-butyl (4-acetylbenzyl(methyl)carbamate

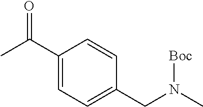 [SMILES: CC(=O)c1ccc(CN(C)C(=O)OC(C)(C)C)cc1]

To a stirred solution of tert-butyl (4-cyanobenzyl)(methyl)carbamate (492 mg, 2 mmol) in THF (10 mL) was added dropwise MeMgBr (3.0 M, 0.8 mL, 2,4 mmol) at 0° C. After then, the mixture was stirred overnight. The reaction was quenched with aq·NH₄Cl, extracted with EtOAc (15 mL×3). The combined organic layers were washed with brine, dried over Na₂SO₄, concentrated to dryness. The resulting residue was purified by flash column chromatography to give the titled compound (213 mg, 40%). MS: M/e 264 (M+1)⁺.

### Step C: tert-butyl (4-(1-hydroxyethyl)benzyl)methyl)carbamate

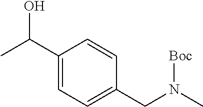 [SMILES: CC(O)c1ccc(CN(C)C(=O)OC(C)(C)C)cc1]

To a stirred solution of tert-butyl (4-acetylbenzyl)(methyl)carbamate (213 mg, 0.81 mmol) in MeOH (5 mL) was added NaBH₄ (30.8 mg, 0.81 mmol). After then, the reaction mixture was stirred for 10 min. The reaction mixture was quenched with H₂O, then most MeOH was removed to give the aqueous layer, then extracted with EtOAc (10 mL×3). The combined organic layers were washed with brine, dried over Na₂SO₄, concentrated to give the titled compound (200 mg, 93.1%). MS: M/e 266 (M+1)⁺.

### Step D: tert-butyl (4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)benzyl)methylcarbamate

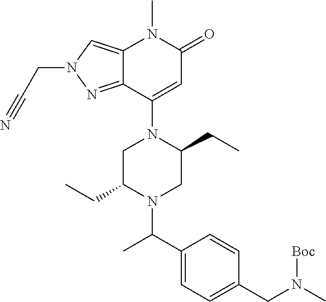 [SMILES: CC[C@H]1CN(C(C)c2ccc(CN(C)C(=O)OC(C)(C)C)cc2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), tert-butyl (4-(1-hydroxyethyl)benzyl)(methyl)carbamate (53.4 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred overnight at 100° C. in a sealed tube. The reaction mixture was diluted with EtOAc (10 mL), then washed with brine, dried over Na₂SO₄, concentrated to dryness. The resulting residue was purified by Pre-TLC (CH₂Cl₂/MeOH=10:1) to give the titled compound (15 mg). MS: M/e 576 (M+1)⁺.

### Step E: 2-(7-((2S,5R)-2,5-diethyl-4-(1-(4-((methylamino)methyl)phenyl))ethyl)piperazin-1-yl)-4-methyl-5-oxo-4,5-dihydro-2H-pyrazolo[4,3-b]pyridin-2-yl)acetonitrile

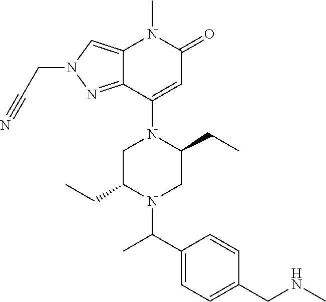 [SMILES: CC[C@H]1CN(C(C)c2ccc(CNC)cc2)[C@H](CC)CN1c1cc(=O)n(C)c2cn(CC#N)nc12]

To a stirred solution of tert-butyl (4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)benzyl)(methyl)carbamate (15 mg, 0.026 mmol) in CH₂Cl₂ (5 mL) was added TFA (2 mL). After then, the mixture was stirred for 2 hours. The reaction mixture was concentrated to give The resulting residue, which was purified by Prep-HPLC (Method A) to give the titled compound (3 mg). ¹H NMR (400 MHz, CD₃OD) δ 8.42 (s, 1H), 7.96-7.90 (m, 1H), 7.57-7.40 (m, 4H), 5.55 (s, 1H), 5.50-5.42 (m, 2H), 4.20-4.14 (m, 2H), 3.85-3.62 (m, 1H), 3.54-3.46 (m, 0.5H), 3.43 (s, 3H), 3.32-3.22 (m, 2,5H), 3.19-3.11 (m, 1H), 3.06-2.84 (m, 1H), 2.75-2.69 (m, 3H), 2.68-2.61 (m, 0.5H), 2,45-2.32 (m, 1H), 2.17-1.48 (m, 4H), 1.40-1,27 (m, 3H), 1.07-0.92 (m, 3H), 0.72-0.58 (m, 3H) ppm. MS: M/e 476 (M+1)⁺.

### Compound A30: 2-(7-((2S,5R)-2,5-diethyl-4-(1-(4-(1-methyl-1H-1,2,3-triazol-4-yl)phenyl)ethyl)piperazin-1-yl)-4-methyl-5-oxo-4,5-dihydro-2H-pyrazolo[4,3-b]pyridin-2-yl)acetonitrile

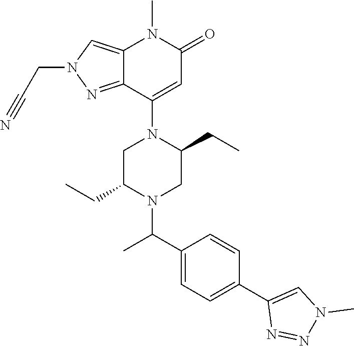 [SMILES: CC[C@H]1CN(C(C)c2ccc(-c3cn(C)nn3)cc2)[C@H](CC)CN1c1cc(=O)n(C)c2cn(CC#N)nc12]

### Step A: 1-(4-(1-methyl-1H-1,23-triazol-4-yl)phenyl)ethan-1-one

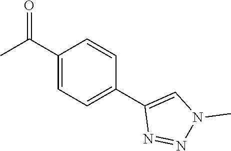

A mixture of (4-acetylphenyl)boronic acid (200 mg, 1,22 mmol), 4-bromo-1-methyl-1H-1,2,3-triazole (196 mg, 1,22 mmol), potassium carbonate (337 mg, 2,44 mmol) and Pd(dppf)Cl₂ (89 mg, 0.12 mmol) in dioxane (3 mL) and water (0.5 mL) was stirred at 100° C. under N₂ overnight. The reaction mixture was diluted with EA and concentrated to dryness. The resulting residue was purified by flash column chromatography to give the titled compound (180 mg, 73%). MS: M/e 202 (M+1)⁺.

### Step B: 1-(4-(1-methyl-1H-1,2.3-triazol-4-yl)phenylethan-1-ol

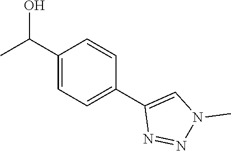

To a solution of 1-(4-(1-methyl-1H-1,2,3-triazol-4-yl)phenyl)ethan-1-one (180 mg, 0.89 mmol) in MeOH (15 mL) was added NaBH₄ (68 mg, 1.79 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (180 mg, 99%). MS: M/e 204 (M+1)⁺.

### Step C: 2-(7-((2S,5R)-2,5-diethyl-4-(1-(4-(1-methyl-1H-1,23-triazol-4-yl)phenyl)ethyl)piperazin-1-yl)-4-methyl-5-oxo-4.-dihydro-2H-pyrazolo[4,3-b]pyridin-2-yl)acetonitrile

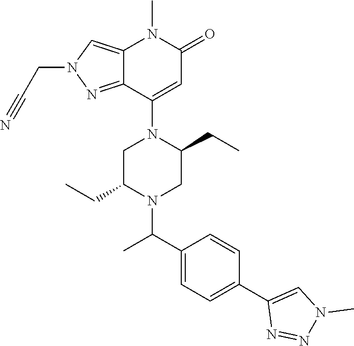 [SMILES: CC[C@H]1CN(C(C)c2ccc(-c3cn(C)nn3)cc2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(4-(1-methyl-1H-1,2,3-triazol-4-yl)phenyl)ethan-1-ol (31 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled compound (21 mg, 26%). ¹H NMR (400 MHz, CD₃OD) δ 8.25 (d, J=4.5 Hz, 1H), 7.92 (d, J=3.4 Hz, 1H), 7.80-7.75 (m, 2H), 7.49-7.44 (m, 2H), 5.55 (s, 1H), 5.46 (d, J=5.2 Hz, 2H), 4.15 (d, J=3.0 Hz, 3H), 3.81-3.62 (m, 1H), 3.51 (d, J=12.2 Hz, 1H), 3.43 (d, J=1.1 Hz, 3H), 3.29-3.17 (m, 2H), 3.16-3.00 (m, J=11.7 Hz, 1H), 2.92-2.63 (m, 1H), 2,52-2.39 (m, 1H), 2.19-1.91 (m, 1H), 1.86-1.52 (m, 3H), 1.36 (dd, J=16.7, 6.5 Hz, 3H), 1.00 (dt, J=30.0, 7.4 Hz, 3H), 0.67 (dt, J=32,4, 7.3 Hz, 3H) ppm. MS: M/e 514 (M+1)⁺.

### Compound A31: 2-(7-((2S,5R)-2,5-diethyl-4-(1-(4-isopropylphenyl)ethyl)piperazin-1-yl)-4-methyl-5-oxo-4,5-dihydro-2H-pyrazolo[4,3-b]pyridin-2-yl)acetonitrile

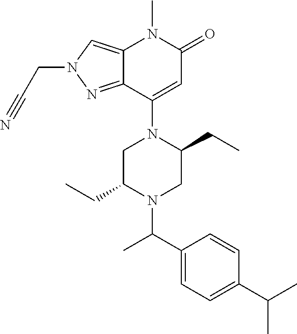 [SMILES: CC[C@H]1CN(C(C)c2ccc(C(C)C)cc2)[C@H](CC)CN1c1cc(=O)n(C)c2cn(CC#N)nc12]

### Step A: 1-(4-isopropylphenyl)ethan-1-ol

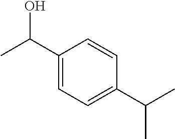

To a solution of 1-(4-isopropylphenyl)ethan-1-one (320 mg, 2 mol) in MeOH was added NaBH₄ (76 mg, 2 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (300 mg, 91%). MS: M/e 165 (M+1)⁺.

### Step B: 2-(7-((2S,5R)-2,5-diethyl-4-(1-(4-isopropylphenyl)ethyl)piperazin-1-yl-4-methyl-5-oxo-4,5-dihydro-2H-pyrazolo[4,3-b]pyridin-2-yl)acetonitrile

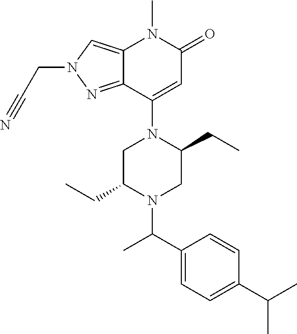 [SMILES: CC[C@H]1CN(C(C)c2ccc(C(C)C)cc2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(4-isopropylphenyl)ethan-1-ol (50 mg, 0.3 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method B) to give the titled compound (8 mg, 17%). ¹H NMR (400 MHz, CD₃OD) δ 7.92 (s, 1H), 7.28 (m, 2H), 7.19 (m, 2H), 5.54 (s, 1H), 5.46 (d, J=3.1 Hz, 2H), 3.69 (s, 0.5H), 3.55 (s, 0.5H), 3.48 (d, J=12.2 Hz, 1H), 3.43 (s, 3H), 3.35 (s, 1H), 3.25 (s, 1H), 3.13 (s, 0.5H), 2.99 (d, J=11.3 Hz, 0.5H), 2.89 (d, J=6.4 Hz, 1.5H), 2,58 (s, 0.5H), 2,41 (m, 1H), 1.88 (m, 1H), 1.57 (m, 3H), 1.33 (m, 3H), 1,24 (t, J=6.6 Hz, 6H), 0.99 (dt, J=24.3, 7.2 Hz, 3H), 0.68 (t, J=7.3 Hz, 1.5H), 0.61 (t, J=7.3 Hz, 1.5H) ppm. MS: M/e 475 (M+1)⁺.

### Compound A32: 2-(7-((2S,5R)-4-(1-(4-cyclopropoxyphenyl)ethyl)-2,5-diethylpiperazin-1-yl)-4-methyl-5-oxo-4,5-dihydro-2H-pyrazolo[4,3-b]pyridin-2-yl)acetonitrile

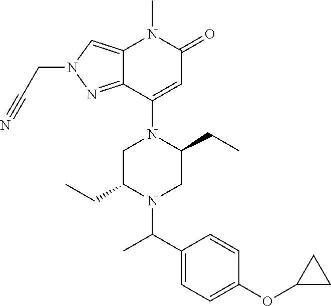 [SMILES: CC[C@H]1CN(C(C)c2ccc(OC3CC3)cc2)[C@H](CC)CN1c1cc(=O)n(C)c2cn(CC#N)nc12]

### Step A: 1-(4-cyclopropoxyphenyl)ethan-1-one

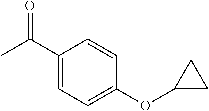

To a solution of 1-(4-hydroxyphenyl)ethan-1-one (1.36 g, 10 mol) in DMF was added bromocyclopropane (1.8 g, 15 mmol) and Cs₂CO₃ (6.5 g, 20 mmol). The reaction mixture was stirred at 100° C. for overnight. The mixture was added H₂O (20 ml) and extracted with EA (20 mL×3). The organic phase was dried with Na₂SO₄ and concentrated by using a rotary evaporator to give a residue. The resulting residue was purified by flash column chromatography to give the titled compound (30 mg, 1.7%). MS: M/e 177 (M+1)

### Step B: 1-(4-cyclopropoxyphenyl)ethan-1-ol

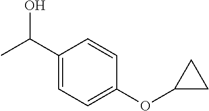

To a solution of 1-(4-cyclopropoxyphenyl)ethan-1-one (30 mg, 0.17 mol) in MeOH was added NaBH₄ (19 mg, 0.5 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (25 mg, 83%). MS: M/e 179 (M+1)⁺.

### Step C: 2-(7-((2,5R-4-(1-(4-cyclopropoxyphenyl)ethyl)-2,5-diethylpiperazin-1-yl)-4-methyl-5-oxo-4,5-dihydro-2H-pyrazolo[4,3-b]pyridazin-2-yl)acetonitrile

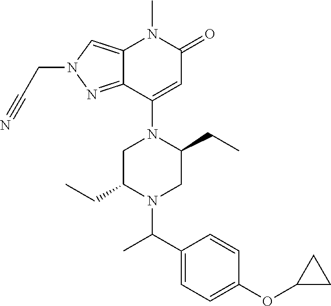 [SMILES: CC[C@H]1CN(C(C)c2ccc(OC3CC3)cc2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(4-cyclopropoxyphenyl)ethan-1-ol (25 mg, 0.14 mmol) and (cyanomethyl)trimethylphosphonium iodide (120 mg, 0.5 mmol) in CH₃CN (10 mL) was added DIPEA (129 mg, 1 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method B) to give the titled compound (2 mg, 5%). ¹H NMR (400 MHz, CD₃OD) δ 7.90 (s, 1H), 7.25 (m, 2H), 6.98 (m, 2H), 5.52 (s, 1H), 5.45 (d, J=3.0 Hz, 2H), 3.74 (d, J=3.1 Hz, 1H), 3.65 (d, J=6.5 Hz, 0.5H), 3.52 (d, J=6.4 Hz, 0.5H), 3.45 (d, J=14.5 Hz, 1H), 3.41 (s, 3H), 3.23 (s, 1H), 3.11 (s, 0.6H), 2.96 (d, J=11.8 Hz, 0.4H), 2.84 (d, J=3.6 Hz, 0.4H), 2,57 (d, J=9.1 Hz, 0.6H), 2.39 (d, J=12,5 Hz, 1H), 2.06 (s, 1H), 1.88 (m, 1H), 1.54 (m, 3H), 1,29 (dd, J=13.9, 6.3 Hz, 3H), 0.98 (dd, J=19.0, 11.7 Hz, 3H), 0.75 (s, 2H), 0.64 (dt, J=24.3, 7.3 Hz, 5H) ppm. MS: M/e 489 (M+1)⁺.

### Compound A33: 2-(7-((2S,5R)-2,5-diethyl-4-(1-(4-isopropoxyphenyl)ethyl)piperazin-1-yl)-4-methyl-5-oxo-4,5-dihydro-2H-pyrazolo[4,3-b]pyridin-2-yl)acetonitrile

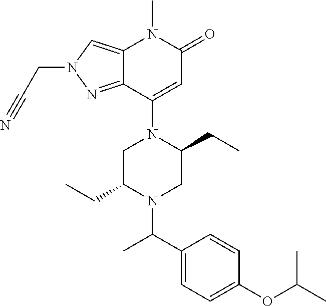 [SMILES: CC[C@H]1CN(C(C)c2ccc(OC(C)C)cc2)[C@H](CC)CN1c1cc(=O)n(C)c2cn(CC#N)nc12]

### Step A: 1-(4-isopropoxyphenyl)ethan-1-one

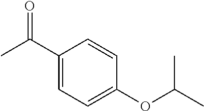

A mixture of 1-(4-hydroxyphenyl)ethan-1-one (680 mg, 5 mmol), 2-iodopropane (1.3 g, 7.5 mmol) and potassium carbonate (1.4 g, 10 mmol) in DMF (10 mL) was stirred at 70° C. overnight. The mixture was treated with water (50 ml), extracted with EtOAc(20 ml*3), washed with brine (50 mL), dried and concentrated to dryness. The resulting residue was purified by flash column chromatography (EtOAc: PE=0-80% in 25 minutes) to give the titled compound (800 mg, 90%). MS: M/e 179 (M+1)⁺.

### Step B: 1-(4-isopropoxyphenyl)ethan-1-ol

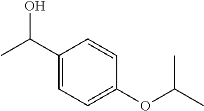

To a solution of 1-(4-isopropoxyphenyl)ethan-1-one (470 mg, 2.6 mmol) in MeOH (5 mL) was added NaBH₄ (79 mg, 2.1 mmol) at room temperature and the mixture was stirred at room temperature for 30 minutes. The resulting mixture was treated with water (50 ml), extracted with DCM (20 mL×2). The combined organic layers were dried over Na₂SO₄ and concentrated to dryness. The resulting residue (450 mg, crude) was used in the next step directly. MS: M/e 181 (M+1)⁺.

### Step C: 2-(7-((2S,5R)-2,5-diethyl-4-(1-(4-isopropoxyphenyl)ethyl)piperazin-1-vi)-4-methyl-5-oxo-4,5-dihydro-2H-pyrazolo[4,3-b]pyridin-2-yl)acetonitrile

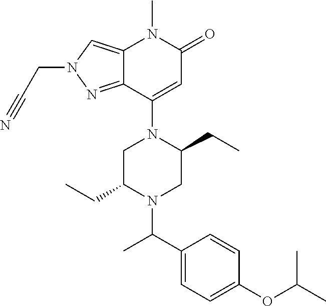 [SMILES: CC[C@H]1CN(C(C)c2ccc(OC(C)C)cc2)[C@H](CC)CN1c1cc(=O)n(C)c2cn(CC#N)nc12]

A mixture of 1-(4-isopropoxyphenyl)ethan-1-ol (270 mg, 1.5 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (164 mg, 0.5 mmol), (cyanomethyl)trimethylphosphonium iodide (363 mg, 1.5 mmol) and DIPEA (322 mg, 2,5 mmol) in MeCN (4 mL) was stirred at 100° C. overnight. The reaction mixture was diluted with EtOAc (20 mL), washed with water (10 mL×2), dried over Na₂SO₄ and concentrated to dryness. The resulting residue was purified by flash column chromatography (MeOH:DCM=0-10% in 30 minutes) to give the compound (−200 mg, crude) which was separated into Compound A33a (49 mg) and Compound A33b (59 mg) by Prep-Chiral-HPLC. The chiral separation conditions are shown below.

| Column | IE |
|---|---|
| Column Size | 20.0 mm × 250 mm, 5 um |
| Mobile Phase A | HEXANE 80% |
| Mobile Phase B | ETOH(0.2% 2M NH₃MEOH) 20% |
| Run Time | 17 min |
| Flow Rate | 18 mL/min |
| Wave Length | UV 200 nm and 310 nm |
| Temperature | RT |
| Prep-HPLC Equipment | Prep-HPLC-Gilson |

Compound A33a (the earlier peak): ¹H NMR (400 MHz, DMSO-d₆) δ 7.94 (s, 1H), 7.19 (d, J=8.5 Hz, 2H), 6.83 (d, J=8.5 Hz, 2H), 5.57 (s, 2H), 5.35 (s, 1H), 4.54 (dt, J=12.1, 6.0 Hz, 1H), 3.65-3.52 (m, 1H), 3.27 (s, 2H), 3.23 (s, 3H), 3.05 (d, J=10.3 Hz, 1H), 2.85 (d, J=11.2 Hz, 1H), 2.69 (d, J=8.3 Hz, 1H), 2.28 (s, 1H), 2.04-1.88 (m, 1H), 1.58 (d, J=6.8 Hz, 1H), 1.39 (d, J=7.3 Hz, 2H), 1,29-1.14 (m, 9H), 0.82 (t, J=7.3 Hz, 3H), 0.57 (t, J=7.2 Hz, 3H) ppm. MS: M/e 491 (M+1)⁺.

Compound A33b (the later peak): ¹H NMR (400 MHz, DMSO-d₆) δ 7.94 (s, 1H), 7.21 (d, J=8.5 Hz, 2H), 6.81 (d, J=8.5 Hz, 2H), 5.58 (s, 2H), 5.34 (s, 1H), 4.53 (dt, J=12.0, 5.9 Hz, 1H), 3.45 (d, J=6.3 Hz, 1H), 3.27 (s, 2H), 3.23 (s, 3H), 2.98 (d, J=9.5 Hz, 1H), 2,42 (s, 2H), 2.23 (d, J=12.2 Hz, 1H), 1.89-1.74 (m, 1H), 1.48 (dd, J=24.2, 16.3 Hz, 3H), 1,26-1.12 (m, 9H), 0.90 (t, J=7.2 Hz, 3H), 0.51 (dd, J=21.3, 14.5 Hz, 3H) ppm. MS: M/e 491 (M+1)⁺.

### Compound A34: 2-(7-((2S,5R)-2,5-diethyl-4-(1-(4-methoxyphenyl)ethyl)piperazin-1-yl)-4-methyl-5-oxo-4,5-dihydro-2H-pyrazolo[4,3-b]pyridin-2-yl)acetonitrile

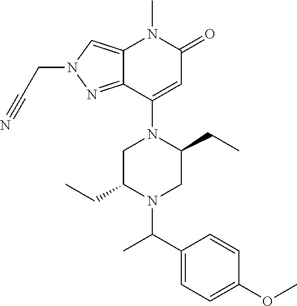 [SMILES: CC[C@H]1CN(C(C)c2ccc(OC)cc2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (300 mg, 0.91 mmol) in CH₃CN (3 mL) and was added 1-(4-methoxyphenyl)ethan-1-ol (139 mg, 0.91 mmol), (cyanomethyl)trimethylphosphonium iodide (670 mg, 2.74 mmol) and DIPEA (590 mg, 4.57 mmol). The resulting mixture was stirred at 105° C. overnight. The reaction solvent was removed under reduced pressure to afford crude product. The resulting residue was purified Prep-HPLC (Method A) to give the titled Compound A34a (60 mg) and Compound A34b (80 mg).

Compound A34a (the earlier peak): ¹H NMR (400 MHz, CD₃OD) δ 7.91 (s, 1H), 7.26 (d, J=8.3 Hz, 2H), 6.89 (d, J=8.3 Hz, 2H), 5.54 (s, 1H), 5.46 (s, 2H), 3.79 (s, 3H), 3.66 (d, J=6.5 Hz, 1H), 3.43 (s, 3H), 3.26 (d, 0.1=13.7 Hz, 2H), 2.98 (d, 0.1=11.2 Hz, 1H), 2.84 (d, 0.1=8.2 Hz, 1H), 2,47-2,40 (m, 1H), 2.13-2.04 (m, 1H), 1.84-1.75 (m, 1H), 1.54-1.45 (m, 2H), 1.34-1,27 (m, 4H), 0.95 (t, J=7.4 Hz, 3H), 0.68 (t, J=7.3 Hz, 3H). MS: M/e 463 (M+1)⁺.

Compound A34b (the later peak): ¹H NMR (400 MHz, CD₃OD) δ 6 7.92 (s, 1H), 7.29 (d, J=8.5 Hz, 2H), 6.86 (d, J=8.5 Hz, 2H), 5.53 (s, 1H), 5.46 (s, 2H), 3.78 (s, 3H), 3.53 (d, J=6.5 Hz, 1H), 3.47 (d, J=12.7 Hz, 1H), 3.43 (s, 3H), 3.29-3.24 (m, 2H), 3.13 (s, 1H), 2,58 (d, J=8.6 Hz, 1H), 2,41 (d, J=12,5 Hz, 1H), 1.94 (d, J=5.9 Hz, 1H), 1.68-1.54 (m, 3H), 1,28 (d, J=6.5 Hz, 3H), 1.01 (t, J=7.4 Hz, 3H), 0.62 (t, J=7.4 Hz, 3H). MS: M/e 463 (M+1)⁺.

### Compound A35: 2-(7-((2S,5R)-4-(1-(4,5-dimethylthiazol-2-yl)ethyl)-2,5-diethylpiperazin-1-yl)-4-methyl-5-oxo-4,5-dihydro-2H-pyrazolo[4,3-b]pyridin-2-yl)acetonitrile

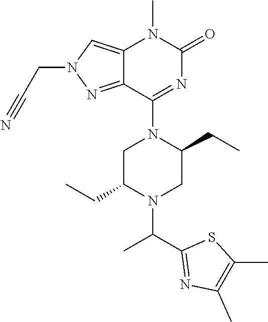 [SMILES: CC[C@H]1CN(C(C)c2nc(C)c(C)s2)[C@H](CC)CN1c1nc(=O)n(C)c2cn(CC#N)nc12]

### Step A: 1-(4.5-dimethylthiazol-2-yl)ethan-1-ol

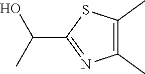

To a −70° C. solution of 2-bromo-4,5-dimethylthiazole (500 mg, 2.6 mmol) in THF (5 mL) was added n-BuLi (1.6 M, 2.0 mL, 3.2 mmol) in drops under N₂. After stirring for 10 minutes at −70° C., a solution of acetaldehyde (350 mg, 8.0 mmol) in THF (1 mL) was added. Then the mixture was allowed warm to room temperature and stirred for 1 hour. Aqueous solution of NH₄C₁ (20 mL) was added and the mixture was extracted with EtOAc (10 mL×3), the organics were combined and washed with brine (10 mL×2), dried over Na₂SO₄, filtered and concentrated to dryness. The resulted residue was purified by column chromatography to give the titled compound (340 mg, 83%). ¹H NMR (500 MHz, DMSO-d₆) δ 5.88 (d, J=5.5 Hz, 1H), 4.85-4.73 (m, 1H), 2.28 (s, 3H), 2.19 (s, 3H), 1.38 (d, J=6.5 Hz, 3H). MS: M/e 158 (M+1)⁺.

### Step B: 247-((2S,5R)-4-(1-44.5-dimethylthiazol-2-yl)ethyl)-2,5-diethylpiperazin-1-yl-4-methyl-5-oxo-4,5-dihydro-2H-pyrazolo[4,3-b]pyridin-2-yl)acetonitrile

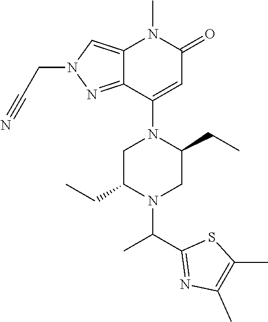 [SMILES: CC[C@H]1CN(C(C)c2nc(C)c(C)s2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), 1-(4,5-dimethylthiazol-2-yl)ethan-1-ol (32 mg, 0.2 mmol) and (cyanomethyl)trimethylphosphonium iodide (85 mg, 0.35 mmol) in CH₃CN (1 mL) was added DIPEA (85 mg, 0.66 mmol). The mixture was stirred at 100° C. in a sealed tube for 20 hours. The mixture was cooled and diluted with EtOAc (10 mL), washed with brine (5 mL×3), dried over Na₂SO₄, filtered and concentrated under reduced pressure. The resulting residue was purified by flash column chromatography and Prep-HPLC (Method A) to give the titled Compound A35a (5 mg) and Compound A35b (5 mg).

Compound A35a (the earlier peak): ¹H NMR (500 MHz, CD₃OD) δ 7.93 (s, 1H), 5.58 (s, 1H), 5.47 (s, 2H), 4.58 (s, 2H), 4.12 (q, J=6.5 Hz, 1H), 3.49-3.35 (m, 4H), 3.00 (dd, J=12.0, 4.0 Hz, 1H), 2.89 (dd, J=12.0, 2.0 Hz, 1H), 2.63 (s, 1H), 2.35 (s, 3H), 2.28 (s, 3H), 2.15-2.03 (m, 1H), 1.90-1.77 (m, 1H), 1.74-1.49 (m, 2H), 1.43 (d, J=7.0 Hz, 3H), 0.94 (t, J=7.5 Hz, 3H), 0.87 (t, J=7.5 Hz, 3H) ppm. MS: M/e 468 (M+1)

Compound A35b (the later peak): ¹H NMR (500 MHz, CD₃OD) δ 7.93 (s, 1H), 5.56 (s, 1H), 5.48 (s, 2H), 4.58 (s, 2H), 3.92 (q, J=6.5 Hz, 1H), 3.50 (d, J=13.5 Hz, 1H), 3.44 (s, 3H), 3.06 (d, J=10.0 Hz, 1H), 2.80 (dd, J=12,5, 4.5 Hz, 1H), 2,56 (dd, J=12,5, 2,5 Hz, 1H), 2.34 (s, 3H), 2.27 (s, 3H), 2.00-1.90 (m, 1H), 1.82-1.69 (m, 1H), 1.69-1.59 (m, 1H), 1.51-1.37 (m, 4H), 0.99 (t, J=7.5 Hz, 3H), 0.79 (t, J=7.5 Hz, 3H) ppm. MS: M/e 468 (M+1)⁺.

### Compound A36: 2-(7-((2S,5R)-2,5-diethyl-4-(1-(4,5,6,7-tetrahydrobenzo[d]thiazol-2-yl)ethyl)piperazin-1-yl)-4-methyl-5-oxo-4,5-dihydro-2H-pyrazolo[4,3-b]pyridin-2-yl)acetonitrile

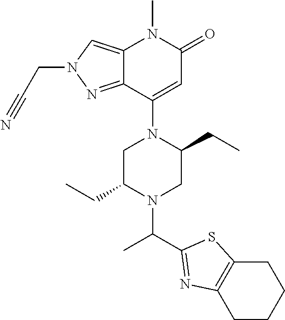 [SMILES: CC[C@H]1CN(C(C)c2nc3c(s2)CCCC3)[C@H](CC)CN1c1cc(=O)n(C)c2cn(CC#N)nc12]

### Step A: 2-bromo-4.5.6.7-tetrahydrobenzo[d]thiazole

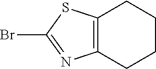

To a 0° C. solution of 4,5,6,7-tetrahydrobenzo[d]thiazol-2-amine (465 mg, 3.0 mmol) in MeCN (6 mL) was added tert-butyl nitrite (620 mg, 6.0 mmol) and followed by CuBr₂ (1.34 g, 6.0 mmol) in portions. The resulted mixture was stirred at room temperature for 2 hours. The mixture was diluted with EA (30 mL) and the suspension was filtered through a celite pad. The filtrate was washed with brine (10 mL×2), dried over Na₂SO₄, and filtered. The filtrate was concentrated under reduced pressure. The resulting residue was purified by flash column chromatography to give the titled compound (300 mg, yield: 46%). ¹H NMR (500 MHz, DMSO-d₆) δ 2.73-2.61 (m, 4H), 1.86-1.69 (m, 4H). MS: M/e 218 (M+1)⁺.

### Step B: 1-(4.5.6.7-tetrahydrobenzo[d]thiazol-2-yl)ethan-1-ol

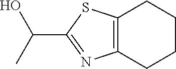

To a −70 “C solution of 2-bromo-4,5,6,7-tetrahydrobenzo[d]thiazole (300 mg, 1.37 mmol) in THF (5 mL) was added n-BuLi (1.6 M, 1.0 mL, 1.6 mmol) in drops under N₂. After stirring for 10 minutes at −70° C., a solution of acetaldehyde (1800 mg, 4.1 mmol) in THF (1 mL) was added. Then the mixture was allowed warm to room temperature and stirred for 1 hour. Aqueous solution of NH₄Cl (20 mL) was added and the mixture was extracted with EtOAc (10 mL×3), the organics were combined and washed with brine (10 mL×2), dried over Na₂SO₄, filtered and concentrated to dryness. The resulted residue was purified by column chromatography to give the titled compound (210 mg, yield: 83%). ¹H NMR (500 MHz, DMSO-d₆) δ 5.91 (d, J=5.0 Hz, 1H), 4.89-4.75 (m, 1H), 2.76-2.66 (m, 2H), 2.65-2,57 (m, 2H), 1.82-1.71 (m, 4H), 1.40 (d, 0.1 =6.5 Hz, 3H). MS: M/e 184 (M+1)”.

### Step C: 2-(7-((2S,5R)-2,5-diethyl-4-(1-(4.5.6.7-tetrahydrobenzo[d]thiazol-2-yl)ethyl)piperazin-1-yl)-4-methyl-5-oxo-4,5-dihydro-2H-pyrazolo[4,3-b]pyridin-2-yl)acetonitrile

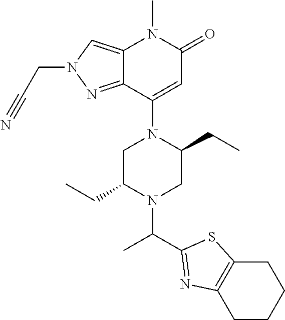 [SMILES: CC[C@H]1CN(C(C)c2nc3c(s2)CCCC3)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), 1-(4,5,6,7-tetrahydrobenzo[d]thiazol-2-yl)ethan-1-ol (37 mg, 0.2 mmol) and (cyanomethyl)trimethylphosphonium iodide (85 mg, 0.35 mmol) in CH₃CN (1 mL) was added DIPEA (85 mg, 0.66 mmol). The mixture was stirred at 100° C. in a sealed tube for 20 hours. The mixture was cooled and diluted with EtOAc (10 mL), washed with brine (5 mL×3), dried over Na₂SO₄, filtered and concentrated under reduced pressure. The resulting residue was purified by flash column chromatography and Prep-HPLC (Method A) to give the titled Compound A36a (5 mg) and Compound A36b (5 mg).

Compound A36a (the earlier peak): ¹H NMR (400 MHz, CD₃OD) δ 7.93 (s, 1H), 5.58 (s, 1H), 5.48 (s, 2H), 4.59 (s, 2H), 4.14 (q, J=6.8 Hz, 1H), 3.49-3.36 (m, 4H), 3.08-2.97 (m, 1H), 2.95-2.84 (m, 1H), 2.83-2.68 (m, 4H), 2.67-2.62 (m, 1H), 2.14-1.96 (m, 1H), 1.94-1.78 (m, 5H), 1.75-1.48 (m, 2H), 1.44 (d, J=6.8 Hz, 3H), 0.94 (t, J=7.6 Hz, 3H), 0.87 (t, J=7.6 Hz, 3H). MS: M/e 494 (M+1)⁺.

Compound A36a (the later peak): ¹H NMR (400 MHz, CD₃OD) δ 7.93 (s, 1H), 5.57 (s, 1H), 5.48 (s, 2H), 4.58 (s, 2H), 4.04-3.88 (m, 1H), 3.58-3.40 (m, 4H), 3.12-3.00 (m, 1H), 2.89-2.64 (m, 5H), 2.62-2,52 (m, 1H), 2.00-1.81 (m, 5H), 1.80-1.54 (m, 2H), 1.51-1.37 (m, 4H), 0.99 (t, J=7.6 Hz, 3H), 0.79 (t, J=7.6 Hz, 3H). MS: M/e 494 (M+1)⁺.

### Compound A37: 2-(7-((2S,5R)-4-(1-(6-cyclopropoxypyridin-3-yl)ethyl)-2,5-diethylpiperazin-1-yl)-4-methyl-5-oxo-4,5-dihydro-2H-pyrazolo[4,3-b]pyridin-2-yl)acetonitrile

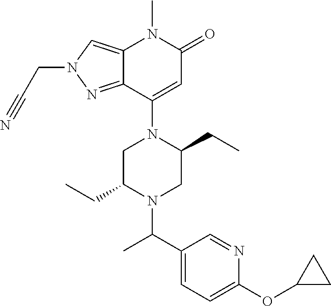 [SMILES: CC[C@H]1CN(C(C)c2ccc(OC3CC3)nc2)[C@H](CC)CN1c1cc(=O)n(C)c2cn(CC#N)nc12]

### Step A: 5-bromo-2-cyclopropoxypyridin

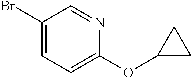 [SMILES: Brc1ccc(OC2CC2)nc1]

To a suspension of Cs₂CO₃ (1.3 g, 4 mmol) in DMF (10 ml) was added 5-bromo-2-fluoropyridine (352 mg, 2 mmol), cyclopropanol (174 mg, 3 mmol) and the resulting reaction mixture stirred at room temperature overnight. The reaction mixture was quenched with water (50 ml) and then extracted with EA(20 ml×3). The combined organic portions were concentrated under reduced pressure and the resulting residue was purified by flash column chromatography (EtOAc: PE=0-50% in 30 minutes) to give the titled compound (350 mg, 82%). MS: M/e 214 (M+1)⁺.

### Step B: 1-(6-cyclopropoxypyridin-3-yl)ethan-1-one

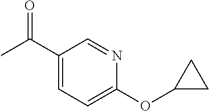

A mixture of 5-bromo-2-cyclopropoxypyridin(350 mg, 1.6 mmol), tributyl(1-ethoxyvinyl)stannane (711 mg, 2 mmol) and Pd(PPh₃)₂Cl₂ (112 mg, 0.16 mmol) in toluene (5 mL) was stirred at 100° C. under N₂ for 16 hours. To the resulting solution was added HCl (0.5 mL, con.) in drops and the mixture was stirred at room temperature for 30 minutes. The mixture was treated with saturated NaHCO₃ aq.(20 ml), extracted with EtOAc(10 ml×2), washed with brine (20 mL), dried and concentrated to dryness. The resulting residue was purified by flash column chromatography (EtOAc: PE=0-50% in 20 minutes) to give the titled compound (90 mg, 32%). MS: M/e 178 (M+1)⁺.

### Step C: 1-(6-cyclopropoxypyridin-3-yl)ethan-1-ol

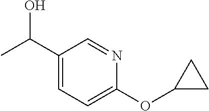

To a solution of 1-(6-cyclopropoxypyridin-3-yl)ethan-1-one (90 mg, 0.51 mmol) in MeOH (3 mL) was added NaBH₄ (15 mg, 0.41 mmol) at room temperature and the mixture was stirred at room temperature for 30 minutes. The resulting mixture was treated with water, extracted with DCM (10 mL×2). The combined organic layers were dried over Na₂SO₄ and concentrated to dryness. The resulting residue (80 mg, crude) was used in the next step. MS: M/e 180 (M+1)⁺.

### Step D: 2-(7-((2S,5R)-4-(1-(6-cyclopropoxypyridin-3-yl)ethyl)-2,5-diethylpiperazin-1-yl)-4-methyl-5-oxo-4,5-dihydro-2H-pyrazolo[4,3-b]pyridin-2-yl)acetonitrile

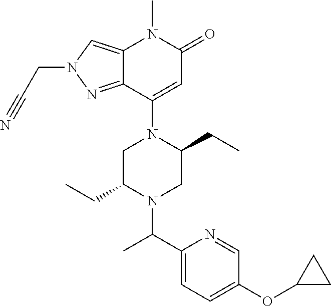 [SMILES: CC[C@H]1CN(C(C)c2ccc(OC3CC3)cn2)[C@H](CC)CN1c1cc(=O)n(C)c2cn(CC#N)nc12]

A mixture of 1-(6-cyclopropoxypyridin-3-yl)ethan-1-ol (27 mg, 0.15 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (48 mg, 0.2 mmol) and DIPEA (51 mg, 0.4 mmol) in MeCN (2 mL) was stirred at 100° C. overnight. The reaction mixture was diluted with EtOAc (20 mL), washed with water (10 mL×2), dried over Na₂SO₄ and concentrated to dryness. The resulting residue was purified by flash column chromatography (MeOH: DCM=0-10% in 20 minutes) to give the titled compound (10 mg, 20%). ¹H NMR (500 MHz, DMSO-d₆) δ 8.12 (s, 1H), 7.98 (d, J=1.7 Hz, 1H), 7.71 (dt, J=8.5, 2,4 Hz, 1H), 6.84 (dd, J=13.5, 8.5 Hz, 1H), 5.62 (d, J=8.4 Hz, 2H), 5.40 (d, J=9.8 Hz, 1H), 4.18 (ddd, J=9.1, 6.0, 3.0 Hz, 1H), 3.75-3.55 (m, 1H), 3.34 (s, 1H), 3.27 (s, 3H), 3.16-3.00 (m, 1H), 2.90-2.74 (m, 1H), 2.33-2.20 (m, 1H), 2.03-1.73 (m, 1H), 1.66-1,20 (m, 7H), 0.97-0.79 (m, 4H), 0.76-0.50 (m, 7H) ppm. MS: M/e 490 (M+1)⁺.

### Compound A38: 2-(7-((2S,5R)-4-(1-(6-cyclopropylpyridin-3-yl)ethyl)-2,5-diethylpiperazin-1-yl)-4-methyl-5-oxo-4,5-dihydro-2H-pyrazolo[4,3-b]pyridin-2-yl)acetonitrile

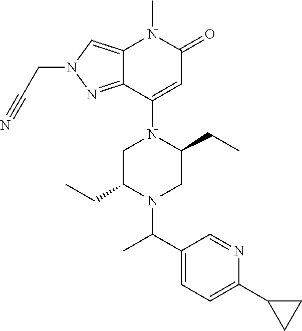 [SMILES: CC[C@H]1CN(C(C)c2ccc(C3CC3)nc2)[C@H](CC)CN1c1cc(=O)n(C)c2cn(CC#N)nc12]

### Step A: 1-(6-cyclopropoxypyridin-3-yl)ethan-1-one

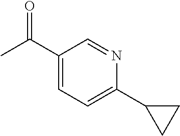

A mixture of 1-(6-bromopyridin-3-yl)ethan-1-one (1 g, 5 mmol), cyclopropylboronic acid (473 mg, 5.5 mmol), tricyclohexylphosphane (140 mg, 0.5 mmol), potassium phosphate (1.6 g, 7.5 mmol) and Pd(OAc)₂ (112 mg, 0.5 mmol) in toluene (15 mL) and water (1.5 ml) was stirred at 100° C. under N₂ overnight. The mixture was treated with water (50 ml), extracted with EtOAc(20 ml×3), washed with brine (50 mL), dried and concentrated to dryness. The resulting residue was purified by flash column chromatography (EtOAc: PE=0-50% in 25 minutes) to give the titled compound (620 mg, 77%). MS: M/e 162 (M+1)⁺.

### Step B: 1-(6-cyclopropylpyridin-3-yl)ethan-1-ol

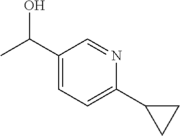

To a solution of 1-(6-cyclopropylpyridin-3-yl)ethan-1-one (620 mg, 3.8 mmol) in MeOH (10 mL) was added NaBH₄ (117 mg, 3.1 mmol) at 0° C. and the mixture was stirred at 0° C. for 30 minutes. The resulting mixture was treated with water(100 ml), extracted with DCM (20 mL×2). The combined organic layers were dried over Na₂SO₄ and concentrated to dryness. The resulting residue was purified by flash column chromatography (EtOAc: PE=0-100% in 25 minutes) to give the titled compound (420 mg, 68%). MS: M/e 164 (M+1)⁺.

### Step C: 2-(7-((2S,5R)-4-(1-(6-cyclopropoxypyridin-3-yl)ethyl-2,5-diethylpiperazin-1-yl)-4-methyl-5-oxo-4,5-dihydro-2H-pyrazolo[4,3-b]pyridin-2-yl)acetonitrile

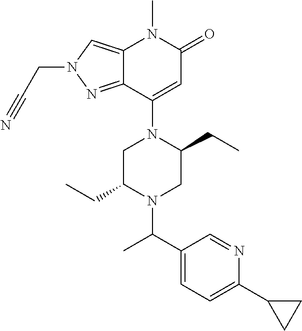 [SMILES: CC[C@H]1CN(C(C)c2ccc(C3CC3)nc2)[C@H](CC)CN1c1cc(=O)n(C)c2cn(CC#N)nc12]

A mixture of 1-(6-cyclopropylpyridin-3-yl)ethan-1-ol (149 mg, 0.91 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (200 mg, 0.6 mmol), (cyanomethyl)trimethylphosphonium iodide (290 mg, 1,2 mmol) and DIPEA (310 mg, 2,4 mmol) in MeCN (5 mL) was stirred at 100° C. overnight. The reaction mixture was diluted with EtOAc (20 mL), washed with water (10 mL×2), dried over Na₂SO₄ and concentrated to dryness. The resulting residue was purified by flash column chromatography (MeOH:DCM=0-10% in 20 minutes) to give the compound (−130 mg, crude) which was separated into Compound A38a (30 mg) and Compound A38b (38 mg) by Prep-Chiral-HPLC. The chiral separation conditions are shown below.

| Column | I-Cellulose-5 |
|---|---|
| Column Size | 20.0 mm × 250 mm, 5 um |
| Mobile Phase A | HEXANE 50% |
| Mobile Phase B | ETOH(0.2% 2M NH₃MEOH) 50% |
| Run Time | 19 min |
| Flow Rate | 18 mL/min |
| Wave Length | UV 200 nm and 310 nm |
| Temperature | RT |
| Prep-HPLC Equipment | Prep-HPLC-Gilson |

Compound A38a (the earlier peak): ¹H NMR (500 MHz, DMSO-d6) δ 8.34 (d, J=1.9 Hz, 1H), 7.97 (s, 1H), 7.60 (dd, J=8.0, 2.2 Hz, 1H), 7.25 (d, J=8.0 Hz, 1H), 5.61 (s, 2H), 5.38 (s, 1H), 3.71 (q, J=6.5 Hz, 1H), 3.32 (s, 1H), 3.28 (d, J=13.6 Hz, 4H), 3.11 (d, 0.1=11.9 Hz, 1H), 2.87 (d, J=11.0 Hz, 1H), 2.76 (dd, J=11.9, 3.8 Hz, 1H), 2.27 (s, 1H), 2.07 (s, 1H), 2.02-1.91 (m, 1H), 1.69-1.56 (m, 1H), 1.51-1.39 (m, 2H), 1,28 (d, J=6.5 Hz, 3H), 0.96-0.80 (m, 7H), 0.62 (t, J=7.3 Hz, 3H) ppm. MS: M/e 474 (M+1)⁺.

Compound A38b (the later peak): ¹H NMR (500 MHz, DMSO-d₆) δ 8.34 (d, J=1.9 Hz, 1H), 7.98 (s, 1H), 7.61 (dd, J=8.0, 2.1 Hz, 1H), 7.24 (d, J=8.0 Hz, 1H), 5.61 (s, 2H), 5.38 (s, 1H), 3.58 (q, J=6.4 Hz, 1H), 3.35 (s, 2H), 3.28 (d, J=13.1 Hz, 4H), 3.03 (d, J=9.9 Hz, 1H), 2,53-2,50 (m, 1H), 2.19 (d, J=12.0 Hz, 1H), 2.11-2.02 (m, 1H), 1.80 (dt, J=16.0, 7.9 Hz, 1H), 1.60-1.39 (m, 3H), 1,24 (d, J=6.5 Hz, 3H), 0.98-0.82 (m, 7H), 0.53 (t, J=7.0 Hz, 3H) ppm. MS: M/e 474 (M+1)⁺.

### Compound A39: 2-(but-2-yn-1-yl)-7-((2S,5R)-4-(1-(6-cyclopropylpyridin-3-yl)ethyl)-2,5-diethylpiperazin-1-yl)-4-methyl-2,4-dihydro-5H-pyrazolo[4,3-b]pyridin-5-one

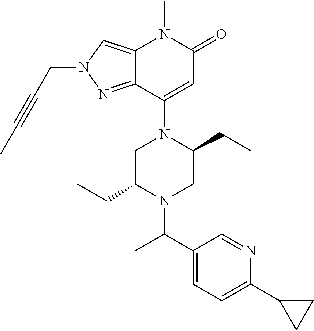 [SMILES: CC#CCn1cc2c(n1)c(N1C[C@@H](CC)N(C(C)c3ccc(C4CC4)nc3)C[C@@H]1CC)cc(=O)n2C]

A mixture of 2-(but-2-yn-1-yl)-7-((2S,5R)-2,5-diethylpiperazin-1-yl)-4-methyl-2,4-dihydro-5H-pyrazolo[4,3-b]pyridin-5-one (102 mg, 0.3 mmol), 1-(6-cyclopropylpyridin-3-yl)ethan-1-ol (99 mg, 0.6 mmol), (cyanomethyl)trimethylphosphonium iodide (219 mg, 0.9 mmol) and DIPEA (192 mg, 1.5 mmol) in CH₃CN (8 mL) was stirred overnight at 100° C. in a sealed tube. The reaction mixture was diluted with EtOAc (15 mL), washed with H₂O, brine, dried over Na₂SO₄, concentrated to dryness. The resulting residue was purified by flash column chromatography to give the titled compound, which was further separated into Compound A39a (24 mg) and Compound A39b (30 mg) by Prep-HPLC (Method B).

Compound A39a (the earlier peak): ¹H NMR (500 MHz, CD₃OD) δ 8.30 (d, J=2.0 Hz, 1H), 7.88 (s, 1H), 7.70 (dd, .=8.0, 2.0 Hz, 1H), 7.20 (d, J=8.0 Hz, 1H), 5.52 (s, 1H), 5.04-5.01 (m, 2H), 3.77 (q, J=6.4 Hz, 1H), 3.43 (s, 3H), 3.33-3.31 (m, 2H), 3.28-3.23 (m, 1H), 2.99-2.83 (m, 2H), 2.38-2.32 (m, 1H), 2.12-2.03 (m, 2H), 1.84 (t, J=2,4 Hz, 3H), 1.83-1.75 (m, 1H), 1.58-1.46 (m, 2H), 1.35 (d, J=6.4 Hz, 3H), 1.06-1.01 (m, 2H), 0.98-0.91 (m, 5H), 0.71 (t, J=7.2 Hz, 3H) ppm. MS: M/e 487 (M+1)⁺.

Compound A39b (the later peak): ¹H NMR (500 MHz, CD₃OD) δ 8.33 (d, J=2.0 Hz, 1H), 7.89 (s, 1H), 7.72 (dd, J=8.0, 2.0 Hz, 1H), 7.17 (d, J=8.0 Hz, 1H), 5.51 (s, 1H), 5.04 (dd, J=4.8, 2,4 Hz, 2H), 3.62 (q, J=6.4 Hz, 1H), 3.49-3.44 (m, 1H), 3.43 (s, 3H), 3.31-3.29 (m, 2H), 3.15-3.09 (m, 1H), 2.67-2.61 (m, 1H), 2.33-2.28 (m, 1H), 2.11-2.05 (m, 1H), 1.95-1.87 (m, 1H), 1.85 (t, J=2,4 Hz, 3H), 1.70-1.48 (m, 3H), 1.31 (d, J=6.4 Hz, 3H), 1.05-0.99 (m, 5H), 0.98-0.89 (m, 2H), 0.62 (t, J=7.2 Hz, 3H) ppm. MS: M/e 487 (M+1)⁺.

### Compound A40: 2-(7-((2S,5R)-4-(1-(6-cyclopropyl-5-fluoropyridin-3-yl)ethyl)-2,5-diethylpiperazin-1-yl)-4-methyl-5-oxo-4,5-dihydro-2H-pyrazolo[4,3-b]pyridin-2-yl)acetonitrile

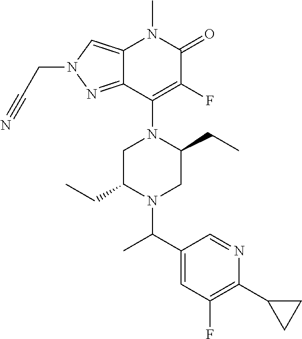 [SMILES: CC[C@H]1CN(C(C)c2cnc(C3CC3)c(F)c2)[C@H](CC)CN1c1c(F)c(=O)n(C)c2cn(CC#N)nc12]

### Step A: methyl 6-cyclopropyl-5-fluoronicotinate

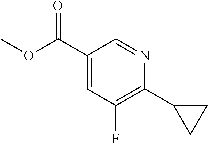

A mixture of methyl 6-bromo-5-fluoronicotinate (1 g, 4.27 mmol), cyclopropylboronic acid (0.55 g, 6.41 mmol) in dioxane (20 mL) was added Pd(dppf)Cl₂ (314 mg, 0.43 mmol), followed by K₂CO₃ (1.18 g, 8.54 mmol). After the addition, the mixture was stirred at 100° C. overnight under N₂. The reaction mixture was treated with H₂O, extracted with EtOAc (15 mL×3). The combined organic layers were washed with brine, dried over Na₂SO₄, concentrated to dryness. The resulting residue was purified by flash column chromatography to give the titled compound (280 mg, 34%). MS: M/e 196 (M+1)⁺.

### Step B: 6-cyclopropyl-5-fluoro-N-methoxy-N-methylnicotinamide

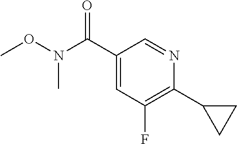 [SMILES: CON(C)C(=O)c1cnc(C2CC2)c(F)c1]

To a stirred solution of methyl 6-cyclopropyl-5-fluoronicotinate (300 mg, 1.54 mmol) in MeOH (8 mL) was added H₂O (2 mL), followed by NaOH (123 mg, 3.07 mmol). After then, the mixture was stirred overnight. The reaction mixture was concentrated to give the aqueous layer, which was acidified to pH=5-6 with aq·HCl, then extracted with EtOAc (10 mL×3). The combined organic layers were washed with brine, dried over Na₂SO₄, concentrated to give the intermediate (245 mg, 87.8%). To a stirred solution of the intermediate in CH₂Cl₂ (10 mL) was added N,O-dimethylhydroxylamine hydrochloride (158 mg, 1.62 mmol), HATU (619 mg, 1.62 mmol) and DIPEA (348.3 mg, 2.7 mmol), then stirred overnight. The reaction mixture was washed with H₂O, extracted with CH₂Cl₂ (15 mL×2). The combined organic layers were washed with brine, dried over Na₂SO₄, concentrated to dryness. The resulting residue was purified by flash column chromatography to give the titled compound (290 mg, 96%). MS: M/e 225 (M+1)⁺.

### Step C: 1-(6-cyclopropyl-5-fluoropyridin-3-yl)ethan-1-one

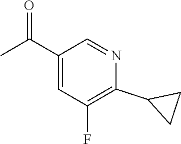

To a stirred solution of 6-cyclopropyl-5-fluoro-N-methoxy-N-methylnicotinamide (290 mg, 1,29 mmol) in THF (10 mL) was added dropwise MeMgBr (3.0 M, 0.56 mL, 1.68 mmol) at 0° C. After then, the mixture was stirred for 30 min. The reaction was quenched with aq·NH₄Cl, extracted with EtOAc (15 mL×3). The combined organic layers were washed with brine, dried over Na₂SO₄, concentrated to dryness. The resulting residue was purified by flash column chromatography to give the titled compound (162 mg, 70%). MS: M/e 180 (M+1)”.

### Step D: 1-(6-cyclopropyl-5-fluoropyridin-3-yl)ethan-1-ol

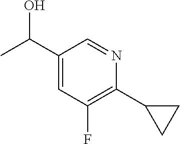

To a stirred solution of 1-(6-cyclopropyl-5-fluoropyridin-3-yl)ethan-1-one (162 mg, 0.9 mmol) in MeOH (5 mL) was added NaBH₄ (34.4 mg, 0.9 mmol). After then, the reaction mixture was stirred for 10 min. The reaction mixture was quenched with H₂O, then most MeOH was removed to give the aqueous layer, then extracted with EtOAc (10 mL×3). The combined organic layers were washed with brine, dried over Na₂SO₄, concentrated to give the titled compound (150 mg, 92%). MS: M/e 182 (M+1)⁺.

### Step E: 2-(7-((2S,5R)-4-(1-(6-cyclopropyl-5-fluoropyridin-3-yl)ethyl)-2,5-diethylpiperazin-1-yl-4-methyl-5-oxo-4,5-dihydro-2H-pyrazolo[4,3-b]pyridin-2-yl)acetonitrile

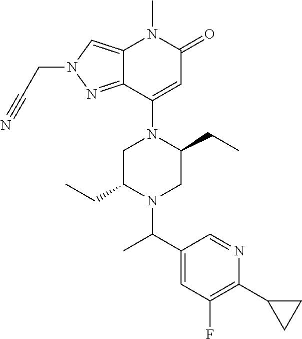 [SMILES: CC[C@H]1CN(C(C)c2cnc(C3CC3)c(F)c2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), 1-(6-cyclopropyl-5-fluoropyridin-3-yl)ethan-1-ol (36.2 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred overnight at 100° C. in a sealed tube. The reaction mixture was diluted with EtOAc (10 mL), then washed with brine, dried over Na₂SO₄, concentrated to dryness. The resulting residue was purified by Pre-TLC(CH₂Cl₂/MeOH=10:1) to give the titled compound (22 mg). ¹H NMR (400 MHz, CD₃OD) δ 8.23-8.15 (m, 1H), 7.93 (s, 1H), 7.58-7.47 (m, 1H), 5.56 (s, 1H), 5.47 (s, 2H), 3.89-3.64 (m, 1H), 3.53-3.46 (m, 0.5H), 3.43 (s, 3H), 3.30-3.26 (m, 2H), 3.18-3.10 (m, 0.5H), 3.00-2.85 (m, 1H), 2.73-2.64 (m, 0.5H), 2,43-2.25 (m, 2H), 2.12-1.76 (m, 1.5H), 1.73-1.46 (m, 3H), 1.42-1.31 (m, 3H), 1.10-0.91 (m, 7H), 0.82-0.61 (m, 3H) ppm. MS: M/e 492 (M+1)⁺.

### Compound A41: 2-(7-((2S,5R)-2,5-diethyl-4-(1-(6-methoxypyridin-3-yl)ethyl)piperazin-1-yl)-4-methyl-5-oxo-4,5-dihydro-2H-pyrazolo[4,3-b]pyridin-2-yl)acetonitrile

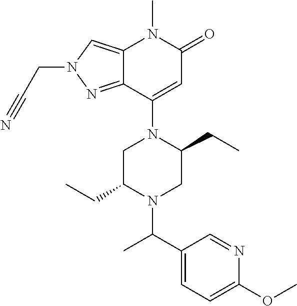 [SMILES: CC[C@H]1CN(C(C)c2ccc(OC)nc2)[C@H](CC)CN1c1cc(=O)n(C)c2cn(CC#N)nc12]

### Step A: 1-(6-methoxypyridin-3-yl)ethan-1-ol

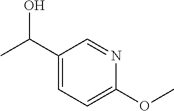

To a solution of 1-(6-methoxypyridin-3-yl)ethan-1-one (302 mg, 2 mmol) in MeOH (5 mL) was added NaBH₄ (61 mg, 1.6 mmol) at room temperature and the mixture was stirred at room temperature for 30 minutes. The resulting mixture was treated with water(50 ml), extracted with DCM (10 mL×2). The combined organic layers were dried over Na₂SO₄ and concentrated to dryness. The resulting residue (250 mg, crude) was used in the next step. MS: M/e 154 (M+1)”.

### Step B: 2-(7-((2S,5R)-2,5-diethyl-4-(1-(6-methoxypyridin-3-yl)ethyl)piperazin-1-yl)-4-methyl-5-oxo-4,5-dihydro-2H-pyrazolo[4,3-b]pyridin-2-yl)acetonitrile

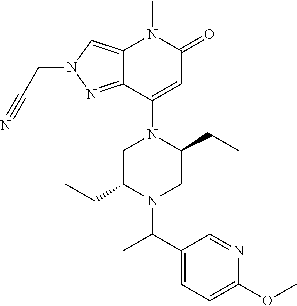 [SMILES: CC[C@H]1CN(C(C)c2ccc(OC)nc2)[C@H](CC)CN1c1cc(=O)n(C)c2cn(CC#N)nc12]

A mixture of 1-(6-methoxypyridin-3-yl)ethan-1-ol (23 mg, 0.15 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (48 mg, 0.2 mmol) and DIPEA (52 mg, 0.4 mmol) in MeCN (2 mL) was stirred at 100° C. overnight. The reaction mixture was diluted with EtOAc (20 mL), washed with water (10 mL×2), dried over Na₂SO₄ and concentrated to dryness. The resulting residue was purified by Prep-HPLC (Method A) to give the titled compound (3.5 mg, 8%). ¹H NMR (500 MHz, CD₃OD) δ 8.05 (dd, J=13.9, 2.2 Hz, 1H), 7.92 (d, J=2.7 Hz, 1H), 7.80-7.70 (m, 1H), 6.81 (dd, J=15.6, 8.6 Hz, 1H), 5.55 (s, 1H), 5.46 (d, J=4.0 Hz, 2H), 3.90 (d, J=5.0 Hz, 3H), 3.80-3.58 (m, 1H), 3.53-3.35 (m, 4H), 3.28-3.09 (m, 2H), 3.01-2.84 (m, 1H), 2.65 (dd, J=8.2, 4.1 Hz, 1H), 2.37 (d, J=12,5 Hz, 1H), 2.12-0.85 (m, 1H), 1.84-1.48 (m, 3H), 1.34 (dd, J=16.4, 6.5 Hz, 3H), 1.05-0.92 (m, 3H), 0.77-0.60 (m, 3H) ppm. MS: M/e 464 (M+1)⁺.

### Compound A42: 2-(7-((2S,5R)-2,5-diethyl-4-(1-(6-(methoxymethyl)pyridin-3-yl)ethyl)piperazin-1-yl)-4-methyl-5-oxo-4,5-dihydro-2H-pyrazolo[4,3-b]pyridin-2-yl)acetonitrile

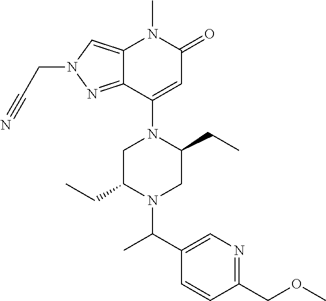 [SMILES: CC[C@H]1CN(C(C)c2ccc(COC)nc2)[C@H](CC)CN1c1cc(=O)n(C)c2cn(CC#N)nc12]

### Step A: 5-bromo-2-(methoxymethyl)pyridine

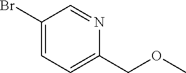

A solution of (5-bromopyridin-2-yl)methanol (1 g, 5.32 mmol) and NaH (60%, 2.66 mg, 6.65 mmol) in THF (10 ml) was stirred at 0° C. for 10 min. Mel (0.94 g, 6.62 mmol) was added to the above solution at 0° C. and then stirred at RT for 1 h. The solution was quenched with water (10 ml) and extracted with EA (10 ml×2). The organic layer was dried over Na₂SO₄ and concentrated to dryness. The resulting residue was purified by flash column chromatography with 0-10% EA in PE to give the titled compound (1 g, 93%). MS: M/e 202 (M+1)⁺.

### Step B: 1-(6-(methoxymethyl)pyridin-3-yl)ethan-1-one

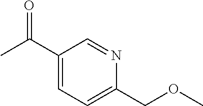

A solution of 5-bromo-2-(methoxymethyl)pyridine (500 mg, 2,49 mmol), ethyl tributylstannanecarboxylate (1.35 g, 3.74 mmol) and Pd(PPh₃)₂Cl₂ (87 mg, 0.12 mmol) in toluene(10 ml) was stirred at 100° C. overnight. The solution was cooled to RT. HCl/1,4-dioxane (4M, 2 ml) was added and stirred at RT for 10 min. The solution was diluted with EtOAc (20 ml), washed with aq. Na₂CO₃ (10 ml×2), dried over Na₂SO₄ and concentrated to dryness. The resulting residue was purified by flash column chromatography with 10/-40/6 EA in PE to give the titled compound (370 mg, 90%). MS: M/e 166 (M+1)⁺.

### Step C: 1-(6-(methoxymethyl)pyridin-3-yl)ethan-1-ol

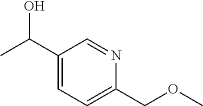

A solution of 1-(6-(methoxymethyl)pyridin-3-yl)ethan-1-one (370 mg, 2.24 mmol) and NaBH₄ (85.2 mg, 2.24 mmol) in MeOH (6 ml) was stirred at RT for 15 min. The solution was diluted with EtOAc (15 ml), washed with brine (10 ml×2), dried over Na₂SO₄ and concentrated to dryness to give the titled compound (330 mg, 89%), which was used directly for the next step without further purification. MS: M/e 168 (M+1)⁺.

### Step D: 2-(7-((2S,5R₁-2,5-diethyl-4-(1-(6-(methoxymethyl)pyridin-3-yl)ethyl)piperazin-1-yl)-4-methyl-5-oxo-4,5-dihydro-2H-pyrazolo[4,3-b]pyridin-2-yl)acetonitrile

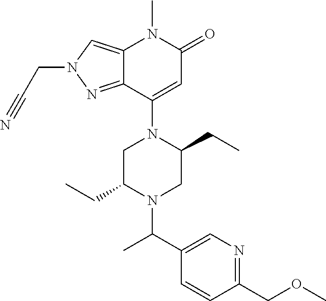 [SMILES: CC[C@H]1CN(C(C)c2ccc(COC)nc2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol), 1-(6-(methoxymethyl)pyridin-3-yl)ethan-1-ol (50.9 mg, 0.30 mmol), (cyanomethyl)trimethylphosphonium iodide (111 mg, 0.46 mmol) and DIPEA (197 mg, 1.53 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A42, which was further separated into Compound A42a (17 mg) and Compound A42b (21 mg) by Prep-HPLC (Method A).

Compound A42a (the earlier peak): ¹H NMR (400 MHz, DMSO-d₆) δ 8.44 (s, 1H), 7.94 (s, 1H), 7.75 (d, J=7.5 Hz, 1H), 7.37 (d, J=7.5 Hz, 1H), 5.57 (s, 2H), 5.35 (s, 1H), 4.45 (s, 2H), 3.75 (s, 1H), 3.32 (s, 5H), 3.23 (s, 3H), 3.09 (d, J=12.6 Hz, 1H), 2.86 (d, J=11.2 Hz, 1H), 2.73 (d, J=12.1 Hz, 1H), 2.23 (s, 1H), 1.95 (s, 1H), 1.58 (s, 1H), 1.42 (s, 2H), 1,26 (s, 3H), 0.81 (s, 3H), 0.58 (s, 3H) ppm. MS: M/e 478 (M+1)⁺.

Compound A42b (the later peak): ¹H NMR (400 MHz, DMSO-d₆) δ 8.45 (s, 1H), 7.94 (s, 1H), 7.76 (d, J=8.3 Hz, 1H), 7.34 (d, J=7.7 Hz, 1H), 5.58 (s, 2H), 5.35 (s, 1H), 4.44 (s, 2H), 3.62 (s, 1H), 3.30 (s, 6H), 3.23 (s, 3H), 3.01 (d, J=8.9 Hz, 1H), 2,51 (d, J=9.1 Hz, 1H), 2.16 (d, J=14.1 Hz, 1H), 1.78 (s, 1H), 1.61-1.34 (m, 3H), 1,22 (d, J=5.6 Hz, 3H), 0.91 (s, 3H), 0.48 (s, 3H) ppm. MS: We 478 (M+1)”.

### Compound A43: 2-(7-((2S,5R)-2,5-diethyl-4-(1-(6-isopropylpyridin-3-yl)ethyl)piperazin-1-yl)-4-methyl-5-oxo-4,5-dihydro-2H-pyrazolo[4,3-b]pyridin-2-yl)acetonitrile

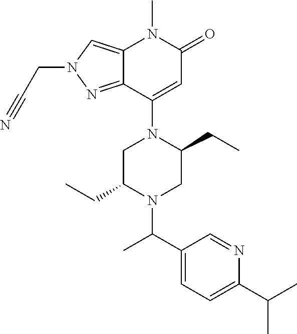 [SMILES: CC[C@H]1CN(C(C)c2ccc(C(C)C)nc2)[C@H](CC)CN1c1cc(=O)n(C)c2cn(CC#N)nc12]

### Step A: 1-(6-isopropylpyridin-3-yl)ethan-1-one

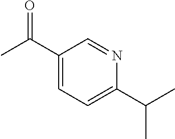

To a stirred solution of 1-(6-bromopyridin-3-yl)ethan-1-one (200 mg, 1 mmol) in dry THF (3 mL) was added XantphosPdG3 (19 mg, 0.02 mmol), followed by isopropyl zinc(II) bromide (0.5 M, 3 mL, 1.5 mmol) under N₂. After the addition, the reaction mixture was stirred at 30° C. overnight in a sealed tube. The reaction mixture was added dropwise to MeOH to quench the reaction. Most solvent was removed to give The resulting residue, then treated with EtOAc (20 mL), and washed with brine, dried over Na₂SO₄, concentrated to dryness. The resulting residue was purified by flash column chromatography to give the titled compound (117 mg, 71.8%). MS: We 164 (M+1)⁺.

### Step B: 146-isopropylpyridin-3-yl)ethan-1-ol

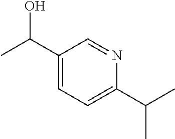

To a stirred solution of 1-(6-isopropylpyridin-3-yl)ethan-1-one (66 mg, 0.4 mmol) in MeOH (5 mL) was added NaBH₄ (15.3 mg, 0.4 mmol). After then, the reaction mixture was stirred for 10 min. The reaction mixture was quenched with H₂O, then most MeOH was removed to give the aqueous layer, then extracted with EtOAc (10 mL×3). The combined organic layers were washed with brine, dried over Na₂SO₄, concentrated to give the titled compound (58 mg, 87.8%). MS: M/e 166 (M+1)⁺.

### Step C: 2-(7-((2S,5R)-2,5-diethyl-4-(1-(6-isopropylpyridin-3-yl)ethyl)piperazin-1-yl)-4-methyl-5-oxo-4,5-dihydro-2H-pyrazolo[4,3-b]pyridin-2-yl)acetonitrile

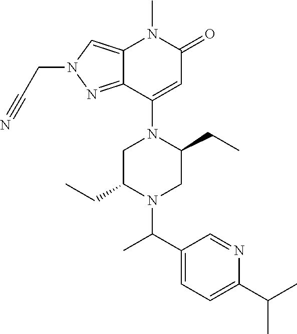 [SMILES: CC[C@H]1CN(C(C)c2ccc(C(C)C)nc2)[C@H](CC)CN1c1cc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), 1-(6-isopropylpyridin-3-yl)ethan-1-ol (33 mg, 0.2 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄, concentrated to dryness. The resulting residue was purified by Pre-TLC (CH₂Cl₂/MeOH=10:1) to give the titled Compound A43, which was further separated into Compound A43a (6 mg) and Compound A43b (5 mg) by Prep-HPLC (Method B).

Compound A43a (the earlier peak): ¹H NMR (500 MHz, CD₃OD) δ 8.40 (d, J=2.0 Hz, 1H), 7.92 (s, 1H), 7.81 (dd, J=8.0, 2.0 Hz, 1H), 7.34 (d, J=8.0 Hz, 1H), 5.56 (s, 1H), 5.46 (s, 2H), 3.87-3.81 (m, 1H), 3.43 (s, 3H), 3.33-3.31 (m, 2H), 3.29-3.26 (m, 1H), 3.12-3.04 (m, 1H), 3.03-2.97 (m, 1H), 2.92-2.86 (m, 1H), 2,41-2.35 (m, 1H), 2.14-2.04 (m, 1H), 1.87-1.77 (m, 1H), 1.61-1.51 (m, 2H), 1.38 (d, J=6.4 Hz, 3H), 1.31 (d, J=6.8 Hz, 6H), 0.95 (t, J=7.2 Hz, 3H), 0.72 (t, J=7.2 Hz, 3H) ppm. MS: M/e 476 (M+1)⁺.

Compound A43b (the later peak): ¹H NMR (500 MHz, CD₃OD) δ 8.44 (d, J=2.0 Hz, 1H), 7.92 (s, 1H), 7.82 (dd, J=8.0, 2.0 Hz, 1H), 7.31 (d, J=8.0 Hz, 1H), 5.55 (s, 1H), 5.47 (s, 2H), 3.71-3.64 (m, 1H), 3.54-3.47 (m, 1H), 3.43 (s, 3H), 3.34-3.31 (m, 2H), 3.20-3.12 (m, 1H), 3.11-3.02 (m, 1H), 2.71-2.65 (m, 1H), 2.35-2.28 (m, 1H), 1.98-1.87 (m, 1H), 1.74-1.51 (m, 3H), 1.35 (d, J=6.4 Hz, 3H), 1,29 (dd, J=6.8, 1,2 Hz, 6H), 1.03 (t, J=7.2 Hz, 3H), 0.61 (t, J=7.2 Hz, 3H) ppm. MS: M/e 476 (M+1)⁺.

### Compound A44: 2-(7-((2S,5R)-2,5-diethyl-4-(1-(6-ethylpyridin-3-yl)ethyl)piperazin-1-yl)-4-methyl-5-oxo-4,5-dihydro-2H-pyrazolo[4,3-b]pyridin-2-yl)acetonitrile

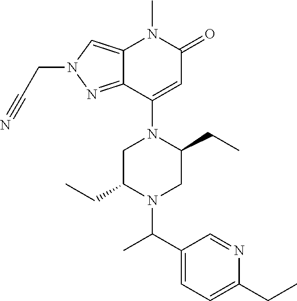 [SMILES: CCc1ccc(C(C)N2C[C@H](CC)N(c3cc(=O)n(C)c4cn(CC#N)nc34)C[C@H]2CC)cn1]

### Step A: 1-(6-vinylpyridin-3-yl)ethan-1-one

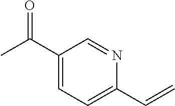

To a solution of 1-(6-bromopyridin-3-yl)ethan-1-one (0.6 g, 3 mmol) and potassium vinyltrifluoroborate (804 mg, 6 mmol) in dioxane/H₂O (15 mL/2 mL) were added X-phosPdG₂ (118 mg, 0.15 mmol) and Cs₂CO₃ (1.96 mg, 6 mmol). The reaction mixture was stirred at 90° C. under N₂ overnight. The mixture was cooled to room temperature, diluted with water (60 mL), extracted with EA (80 mL×2), washed with brine, dried over Na₂SO₄, filtered, and concentrated to dryness. The resulting residue was purified by flash column chromatography to give the titled compound (320 mg, 72%). MS: M/e 148 (M+1)⁺.

### Step B: 1-(6-ethylpyridin-3-yl)ethan-1-one

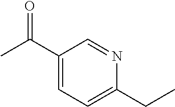

To a solution of 1-(6-vinylpyridin-3-yl)ethan-1-one (320 mg, 2.17 mmol) in MeOH (5 mL) was added 10% Pd/C (33 mg). The reaction mixture was stirred at room temperature under H₂ (balloon) overnight. The mixture was filtered through a pad of Celtic and the filtrate was concentrated to dryness. The resulting residue was used directly for next step (300 mg, crude). MS: M/e 150 (M+1)⁺.

### Step C: 1-(6-ethylpyridin-3-yl)ethan-1-ol

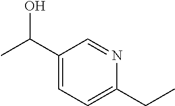

To a solution of 1-(6-ethylpyridin-3-yl)ethan-1-one (300 mg, crude) in MeOH (5 mL) was added NaBH₄ (114 mg, 3 mmol) slowly at 0° C. The resulting mixture was stirred at room temperature for 1 hour. The reaction mixture was quenched with H₂O (10 mL) and extracted with EA (60 mL×2), washed with brine, dried over Na₂SO₄, filtered, and concentrated to dryness. The resulting residue was purified by flash column chromatography to give the desired product (150 mg, 46% for two steps). ¹H NMR (400 MHz, DMSO-d₆) δ 8.38 (d, J=2.0 Hz, 1H), 7.60 (dd, J=2.0 Hz, 8.0 Hz, 1H), 7.16 (d, J=8.0 Hz, 1H), 5.19 (d, J=4.4 Hz, 1H), 4.75-4.66 (m, 1H), 2.72-2.62 (m, 2H), 1.30 (d, J=6.4 Hz, 3H), 1.17 (t, J=8.0 Hz, 3H) ppm.

### Step D: 2-(7-((2S,5R-2,5-diethyl-4-(1-(6-ethylpyridin-3-yl)ethyl)piperazin-1-yl)-4-methyl-5-oxo-4,5-dihydro-2H-pyrazolo[4,3-b]pyridin-2-yl)acetonitrile

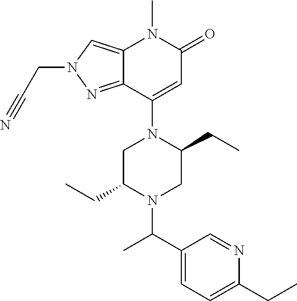 [SMILES: CCc1ccc(C(C)N2C[C@H](CC)N(c3cc(=O)n(C)c4cn(CC#N)nc34)C[C@H]2CC)cn1]

To a solution of 1-(6-ethylpyridin-3-yl)ethan-1-ol (30 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.6 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-HPLC (Method A) to give the titled Compound A44a (6 mg) and Compound A44b (6 mg).

Compound A44a (the earlier peak): ¹H NMR (400 MHz, CD₃OD) δ 8.38 (s, 1H), 7.90 (s, 1H), 7.80 (dd, J=2.0 Hz, 8.4 Hz, 1H), 7.32 (d, J=8.4 Hz, 1H), 5.54 (s, 1H), 5.44 (s, 2H), 3.90-3.76 (m, 1H), 3.41 (s, 3H), 3.35-3.31 (m, 2H), 3.28-3.24 (m, 1H), 3.02-2.95 (m, 1H), 2.93-2.75 (m, 3H), 2,40-2.31 (m, 1H), 2.15-2.00 (m, 1H), 1.86-1.71 (m, 1H), 1.60-1.45 (m, 2H), 1.36 (d, J=6.4 Hz, 3H), 1,28 (t, J=7.6 Hz, 3H), 0.93 (t, J=7.2 Hz, 3H), 0.71 (t, J=7.2 Hz, 3H) ppm. MS: M/e 462 (M+1)⁺.

Compound A44b (the later peak): ¹H NMR (400 MHz, CD₃OD) δ 8.42 (s, 1H), 7.91 (s, 1H), 7.80 (dd, J=2.0 Hz, 8.0 Hz, 1H), 7.29 (d, J=8.4 Hz, 1H), 5.53 (s, 1H), 5.45 (s, 2H), 3.70-3.60 (m, 1H), 3.52-3.44 (m, 1H), 3.41 (s, 3H), 3.35-3.31 (m, 2H), 3.19-3.08 (m, 1H), 2.84-2.72 (m, 2H), 2.71-2.60 (m, 1H), 2.35-2.26 (m, 1H), 1.96-1.79 (m, 1H), 1.71-1.45 (m, 3H), 1.32 (d, J=6.8 Hz, 3H), 1,27 (t, J=7.6 Hz, 3H), 1.01 (t, J=6.8 Hz, 3H), 0.60 (t, J=7.6 Hz, 3H) ppm. MS: M/e 462 (M+1)⁺.

### Compound A45: 2-(7-((2S,5R)-4-(1-(6-cyclopropyl-2-fluoropyridin-3-yl)ethyl)-2,5-diethylpiperazin-1-yl)-4-methyl-5-oxo-4,5-dihydro-2H-pyrazolo[4,3-b]pyridin-2-yl)acetonitrile

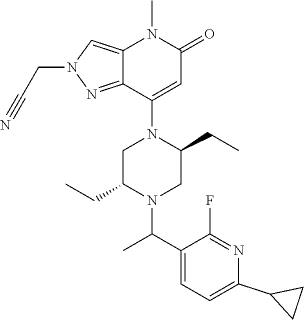 [SMILES: CC[C@H]1CN(C(C)c2ccc(C3CC3)nc2F)[C@H](CC)CN1c1cc(=O)n(C)c2cn(CC#N)nc12]

### Step A: 6-cyclopropyl-2-fluoronicotinaldehyde

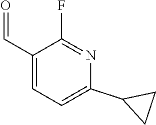 [SMILES: O=Cc1ccc(C2CC2)nc1F]

To a solution of 6-chloro-2-fluoronicotinaldehyde (0.32 g, 2 mmol) and cyclopropylboronic acid (258 mg, 3 mmol) in toluene/H₂O (10 mL/1 mL) were added dichlorobis(tricyclohexylphosphine)palladium(II) (147 mg, 0.2 mmol) and KPO₄ (842 mg, 4 mmol). The reaction mixture was stirred at 100° C. under N₂ overnight. The mixture was cooled to room temperature, diluted with water (40 mL), extracted with EA (60 mL×2), washed with brine, dried over Na₂SO₄, filtered, and concentrated to dryness. The resulting residue was purified by flash column chromatography to give the titled compound (130 mg, 39%). MS: M/e 166 (M+1)⁺.

### Step B: 1-(6-cyclopropyl-2-fluoropyridin-3-yl)ethan-1-ol

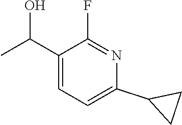

To a solution of 6-cyclopropyl-2-fluoronicotinaldehyde (130 mg, 0.78 mmol) in THF (5 mL) was added a solution of CH—MgBr in ether (0.3 mL, 0.9 mmol) slowly at 0° C. The resulting mixture was stirred at room temperature for 1 hour. The reaction mixture was quenched with saturated NH₄Cl (3 mL) and H₂O (10 mL) and extracted with EA (50 mL×2), washed with brine, dried over Na₂SO₄, filtered, and concentrated to dryness. The resulting residue was purified by flash column chromatography to give the desired product (90 mg, 63%). ¹H NMR (400 MHz, DMSO-d₆) δ 7.84-7.76 (m, 1H), 7.10 (d, J=7.2 Hz, 1H), 5.31 (d, J=4.0 Hz, 1H), 4.85-4.74 (m, 1H), 2.08-1.98 (m, 1H), 1,27 (d, J=6.4 Hz, 3H), 0.96-0.87 (m, 2H), 0.84-0.75 (m, 2H) ppm.

### Step C: 2-(7-((2S,5R)-4-(1-(6-cyclopropyl-2-fluoropyridin-3-yl)ethyl)-2,5-diethylpiperazin-1-yl-4-methyl-5-oxo-4,5-dihydro-2H-pyrazolo[4,3-b]pyridin-2-yl)acetonitrile

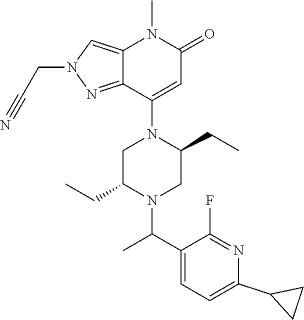 [SMILES: CC[C@H]1CN(C(C)c2ccc(C3CC3)nc2F)[C@H](CC)CN1c1cc(=O)n(C)c2cn(CC#N)nc12]

To a solution of 1-(6-cyclopropyl-2-fluoropyridin-3-yl)ethan-1-ol (36 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.6 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-HPLC (Method A) to give the titled Compound A45a (1 mg) and Compound A45b (1.3 mg).

Compound A45a (The earlier peak): ¹H NMR (400 MHz, CD₃OD) δ 7.91 (s, 1H), 7.89-7.80 (m, 1H), 7.16 (d, J=7.6 Hz, 1H), 5.54 (s, 1H), 5.45 (s, 2H), 4.10-3.95 (m, 1H), 3.49-3.42 (m, 1H), 3.41 (s, 3H), 3.35-3.31 (m, 2H), 2.98-2.80 (m, 2H), 2,49-2.33 (m, 1H), 2.10-1.95 (m, 2H), 1.85-1.70 (m, 1H), 1.58-1.46 (m, 2H), 1.35 (d, J=5.6 Hz, 3H), 1.05-0.85 (m, 7H), 0.76 (t, J=6.8 Hz, 3H) ppm. MS: M/e 492 (M+1)⁺.

Compound A45b (The later peak): ¹H NMR (400 MHz, CD₃OD) δ 7.95-7.85 (m, 2H), 7.14 (d, J=8.0 Hz, 1H), 5.54 (s, 1H), 5.45 (s, 2H), 3.91-3.80 (m, 1H), 3.51-3.42 (m, 1H), 3.41 (s, 3H), 3.35-3.31 (m, 2H), 3.13-3.08 (m, 1H), 2.72-2.62 (m, 1H), 2.35-2.25 (m, 1H), 2.06-1.85 (m, 2H), 1.75-1.40 (m, 3H), 1,29 (d, J=6.0 Hz, 3H), 1.05-0.85 (m, 7H), 0.65 (t, J=7.6 Hz, 3H) ppm. MS: M/e 492 (M+1)⁺.

### Compound A46: 2-(7-((2S,5R)-2,5-diethyl-4-(1-(6-(2-fluoropropan-2-yl)pyridin-3-yl)ethyl)piperazin-1-yl)-4-methyl-5-oxo-4,5-dihydro-2H-pyrazolo[4,3-b]pyridin-2-yl)acetonitrile

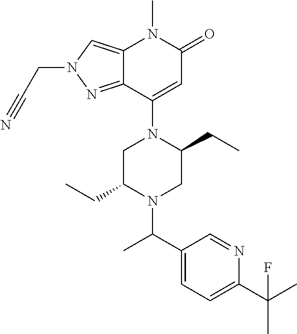 [SMILES: CC[C@H]1CN(C(C)c2ccc(C(C)(C)F)nc2)[C@H](CC)CN1c1cc(=O)n(C)c2cn(CC#N)nc12]

### Step A: methyl 6-acetylnicotinate

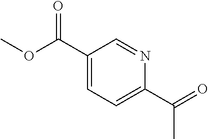

A mixture of methyl 6-chloronicotinate (3.43 g, 20 mmol), tributyl(1-ethoxyvinyl)stannane (10.8 g, 30 mmol) and Pd(PPh₃)Cl₂ (1.4 g, 2 mmol) in toluene (80 mL) was stirred overnight under N₂ at 100° C. The reaction mixture was allowed cool to R.T. and washed with aq·HCl (2.0 M, 20 mL) for 30 min. The organic layer was separated and the aqueous layer was extracted with EtOAc (20 mL×2). The combined organic layers were washed with brine, dried over Na₂SO₄, concentrated to dryness. The resulting residue was purified by flash column chromatography to give the titled compound (1,2 g, 33.5%). MS: M/e 180 (M+1)⁺.

### Step B: methyl 6-(2-hydroxypropan-2-yl)nicotinate

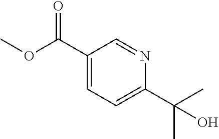

To a stirred solution of methyl 6-acetylnicotinate (358 mg, 2 mmol) in THF (20 mL) was added dropwise MeMgBr (3.0 M, 1.33 mL, 4 mmol) at 0° C. After the addition, the reaction mixture was stirred for 20 min. The reaction was quenched with aq·NH₄Cl, extracted with EtOAc (20 mL×3). The combined organic layers were washed with brine, dried over Na₂SO₄, concentrated to dryness. The resulting residue was purified by flash column chromatography to give the titled compound (280 mg, 57%). MS: M/e 196 (M+1)⁺.

### Step C: methyl 6-(2-fluoropropan-2-yl)nicotinate

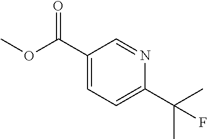

To a stirred solution of methyl 6-(2-hydroxypropan-2-yl)nicotinate (273 mg, 1.4 mmol) in CH₂Cl₂ (10 mL) was added dropwise a solution of DAST (451 mg, 2.8 mmol) in CH₂Cl₂ (3 mL) at 0° C. After the addition, the reaction was stirred for 3 hours. The reaction was quenched with aq. NalHCO₃, then extracted with CH₂Cl₂ (10 mL×2). The combined organic layers were washed with brine, dried over Na₂SO₄, concentrated to dryness. The resulting residue was purified by flash column chromatography to give the titled compound (225 mg, 81.6%). MS: We 198 (M+1)⁺.

### Step D: 6-(2-fluoropropan-2-yl)nicotinic acid

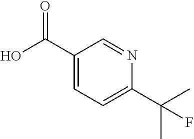

To a stirred solution of methyl 6-(2-fluoropropan-2-yl)nicotinate (225 mg, 1.14 mmol) in MeOH (5 mL) was added aq. NaOH (2.0 M, 2 mL). After then, the mixture was stirred overnight. The reaction mixture was acidified to pH=5-6 with aq·HCl, then concentrated to give the crude product, which was directly used to the next step. MS: M/e 184 (M+1)⁺.

### Step E: 6-(2-fluoropronan-2-yl-N-methoxy-N-methylnicotinamide

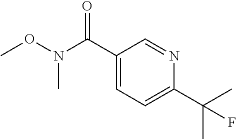 [SMILES: CON(C)C(=O)c1ccc(C(C)(C)F)nc1]

To a stirred solution of 6-(2-fluoropropan-2-yl)nicotinic acid (crude, 1.14 mmol), N,O-dimethylhydroxylamine hydrochloride (133 mg, 1.37 mmol), HATU (523 mg, 1.37 mmol) and DIPEA (294 mg, 2.28 mmol) was stirred overnight. The reaction mixture was washed with H₂O, extracted with CH₂Cl₂ (15 mL×2). The combined organic layers were washed with brine, dried over Na₂SO₄, concentrated to dryness. The resulting residue was purified by flash column chromatography to give the titled compound (223 mg, 86.5%). MS: M/e 227 (M+1)⁺.

### Step F: 1-(6-(2-fluoropropan-2-yl)pyridin-3-yl)ethan-1-one

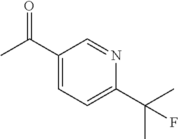

To a stirred solution of 6-(2-fluoropropan-2-yl)-N-methoxy-N-methylnicotinamide (223 mg, 0.986 mmol) in THF (10 mL) was added dropwise MeMgBr (3.0 M, 0.5 mL, 1.45 mmol) at 0° C. After then, the mixture was stirred for 30 min. The reaction was quenched with aq·NH₄Cl, extracted with EtOAc (15 mL×3). The combined organic layers were washed with brine, dried over Na₂SO₄, concentrated to dryness. The resulting residue was purified by flash column chromatography to give the titled compound (120 mg, 67.2%). MS: M/e 182 (M+1)⁺.

### Step G: 1-(6-(2-fluoropropan-2-yl)pyridin-3-yl)ethan-1-ol

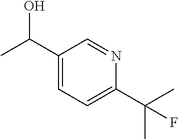

To a stirred solution of 1-(6-(2-fluoropropan-2-yl)pyridin-3-yl)ethan-1-one (120 mg, 0.663 mmol) in MeOH (5 mL) was added NaBH₄ (25.2 mg, 0.663 mmol). After then, the reaction mixture was stirred for 10 min. The reaction mixture was quenched with H₂O, then most MeOH was removed to give the aqueous layer, then extracted with EtOAc (10 mL×2). The combined organic layers were washed with brine, dried over Na₂SO₄, concentrated to give the titled compound (120 mg, 99%). MS: We 184 (M+1)⁺.

### Step H: 2-(7-((2S,5R)-2,5-diethyl-4-(1-(6-(2-fluoropropan-2-yl)pyridin-3-yl)ethyl)piperazin-1-yl)-4-methyl-5-oxo-4,5-dihydro-2H-pyrazolo[4,3-b]pyridin-2-yl)acetonitrile

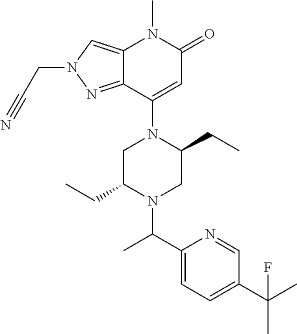 [SMILES: CC[C@H]1CN(C(C)c2ccc(C(C)(C)F)cn2)[C@H](CC)CN1c1cc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), 1-(6-(2-fluoropropan-2-yl)pyridin-3-yl)ethan-1-ol (36.6 mg, 0.2 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄, concentrated to dryness. The resulting residue was purified by Pre-TLC (CH₂Cl₂/MeOH=10:1) to give the titled compound, which was further separated into Compound A46a (4 mg) and Compound A46b (4 mg) by Prep-HPLC (Method B).

Compound A46a (the earlier peak): ¹H NMR (400 MHz, CD₃OD) ¹H NMR (400 MHz, CD₃OD) δ 8.47 (s, 1H), 7.90 (s, 1H), 7.87 (d, J=8.0 Hz, 1H), 7.58 (d, J=8.0 Hz, 1H), 5.54 (s, 1H), 5.44 (s, 2H), 3.90-3.83 (m, 1H), 3.41 (s, 3H), 3.33-3.25 (m, 3H), 3.02-2.86 (m, 2H), 2,41-2.33 (m, 1H), 2.12-2.03 (m, 1H), 1.88-1.76 (m, 1H), 1.70 (s, 3H), 1.65 (s, 3H), 1.60-1.48 (m, 2H), 1.37 (d, J=6.4 Hz, 3H), 0.93 (t, J=7.2 Hz, 3H), 0.71 (t, J=7.2 Hz, 3H) ppm. MS: M/e 494 (M+1)⁺.

Compound A46b (the later peak): ¹H NMR (400 MHz, CD₃OD) δ 8.50 (s, 1H), 7.91 (s, 1H), 7.87 (d, J=8.0 Hz, 1H), 7.55 (d, J=8.0 Hz, 1H), 5.53 (s, 1H), 5.45 (s, 2H), 3.73-3.66 (m, 1H), 3.53-3.45 (m, 1H), 3.41 (s, 3H), 3.33-3.25 (m, 2H), 3.19-3.11 (m, 1H), 2.71-2.64 (m, 1H), 2.33-2.24 (m, 1H), 1.97-1.85 (m, 1H), 1.73-1.60 (m, 8H), 1.59-1.49 (m, 1H), 1.34 (d, J=6.4 Hz, 3H), 1.01 (t, J=7.2 Hz, 3H), 0.59 (t, J=7.2 Hz, 3H) ppm. MS: M/e 494 (M+1)⁺.

### Compound A47: 2-(7-((2S,5R)-4-(1-(6-cyclopropyl-4-fluoropyridin-3-yl)ethyl)-2,5-diethylpiperazin-1-yl)-4-methyl-5-oxo-4,5-dihydro-2H-pyrazolo[4,3-b]pyridin-2-yl)acetonitrile

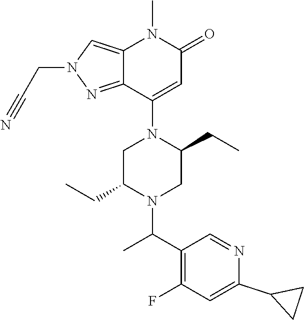 [SMILES: CC[C@H]1CN(C(C)c2cnc(C3CC3)cc2F)[C@H](CC)CN1c1cc(=O)n(C)c2cn(CC#N)nc12]

### Step A: 6-cyclopropyl-4-fluoronicotinaldehyde

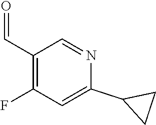 [SMILES: O=Cc1cnc(C2CC2)cc1F]

To a solution of 6-chloro-4-fluoronicotinaldehyde (0.32 g, 2 mmol) and cyclopropylboronic acid (258 mg, 3 mmol) in toluene/H₂O (10 mL/1 mL) were added dichlorobis(tricyclohexylphosphine)palladium(II) (147 mg, 0.2 mmol) and K₃PO₄ (842 mg, 4 mmol). The reaction mixture was stirred at 100° C. under N₂ overnight. The mixture was cooled to room temperature, diluted with water (40 mL), extracted with EA (60 mL×2), washed with brine, dried over Na₂SO₄, filtered, and concentrated to dryness. The resulting residue was purified by flash column chromatography to give the titled compound (200 mg, 60%). MS: M/e 166 (M+1)⁺.

### Step B: 1-(6-cyclopropyl-4-fluoropyridin-3-yl)ethan-1-ol

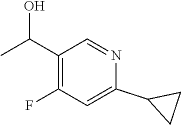

To a solution of 6-cyclopropyl-4-fluoronicotinaldehyde (200 mg, 1,22 mmol) in THF (5 mL) was added a solution of CH₃MgBr in ether (0.4 mL, 1,2 mmol) slowly at 0° C. The resulting mixture was stirred at room temperature for 1 hour. The reaction mixture was quenched with saturated NH₄Cl (3 mL) and H₂O (10 mL) and extracted with EA (50 mL×2), washed with brine, dried over Na₂SO₄, filtered, and concentrated to dryness. The resulting residue was purified by flash column chromatography to give the desired product (150 mg, 68%). ¹H NMR (500 MHz, DMSO-d₆) δ 8.45 (d, J=10.5 Hz, 1H), 7.13 (d, J=11.5 Hz, 1H), 5.35 (d, J=4.0 Hz, 1H), 4.96-4.88 (m, 1H), 2.12-2.04 (m, 1H), 1.36 (d, J=6.5 Hz, 3H), 0.98-0.86 (m, 4H) ppm.

### Step C: 2-(7-((2S,5R)-4-(1-(6-cyclopropyl-4-fluoropyridin-3-yl)ethyl)-2,5-diethylpiperazin-1-yl)-4-methyl-5-oxo-4,5-dihydro-2H-pyrazolo[4,3-b]pyridin-2-yl)acetonitrile

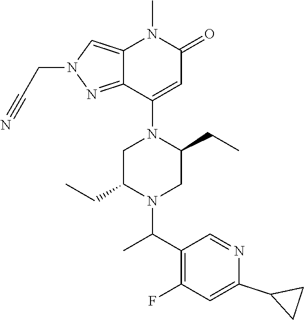 [SMILES: CC[C@H]1CN(C(C)c2cnc(C3CC3)cc2F)[C@H](CC)CN1c1cc(=O)n(C)c2cn(CC#N)nc12]

To a solution of 1-(6-cyclopropyl-4-fluoropyridin-3-yl)ethan-1-ol (36 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.6 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified and separated into Compound A47a (10 mg) and Compound A47b (12 mg) by Prep-HPLC (Method A).

Compound A47a (the earlier peak): ¹H NMR (500 MHz, CD₃OD) δ 8.45 (d, J=10.0 Hz, 1H), 7.92 (s, 1H), 7.00 (d, J=11.5 Hz, 1H), 5.56 (s, 1H), 5.46 (s, 2H), 4.16-4.10 (m, 1H), 3.44 (s, 3H), 3.32-3.27 (m, 3H), 2.96-2.88 (m, 2H), 2,44-2.36 (m, 1H), 2.14-1.98 (m, 2H), 1.85-1.70 (m, 1H), 1.60-1.46 (m, 2H), 1.41 (d, J=7.0 Hz, 3H), 1.09-0.89 (m, 7H), 0.77 (t, J=7.5 Hz, 3H) ppm. MS: M/e 492 (M+1)⁺.

Compound A47b (the later peak): ¹H NMR (500 MHz, CD₃OD) δ 8.53 (d, J=9.5 Hz, 1H), 7.92 (s, 1H), 6.97 (d, J=11.0 Hz, 1H), 5.56 (s, 1H), 5.47 (s, 2H), 4.01-3.93 (m, 1H), 3.53-3.45 (m, 1H), 3.43 (s, 3H), 3.35-3.31 (m, 2H), 3.13-3.10 (m, 1H), 2.75-2.68 (m, 1H), 2.39-2.32 (m, 1H), 2.13-2.06 (m, 1H), 1.98-1.88 (m, 1H), 1.74-1.60 (m, 2H), 1.57-1.48 (m, 1H), 1.37 (d, J=6.5 Hz, 3H), 1.07-0.90 (m, 7H), 0.65 (t, J=7.5 Hz, 3H) ppm. MS: M/e 492 (M+1)⁺.

### Compound A48: 2-(7-((2S,5R)-4-(1-(6-cyclopropylpyridin-3-yl)ethyl)-2,5-diethylpiperazin-1-yl)-6-fluoro-4-methyl-5-oxo-4,5-dihydro-2H-pyrazolo[4,3-b]pyridin-2-yl)acetonitrile

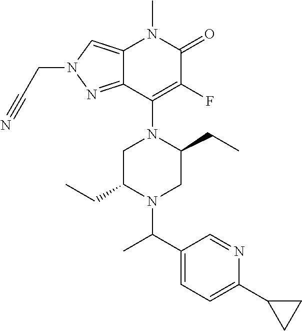 [SMILES: CC[C@H]1CN(C(C)c2ccc(C3CC3)nc2)[C@H](CC)CN1c1c(F)c(=O)n(C)c2cn(CC#N)nc12]

To a solution of 2-(7-((2S,5R)-4-(1-(6-cyclopropylpyridin-3-yl)ethyl)-2,5-diethylpiperazin-1-yl)-4-methyl-5-oxo-4,5-dihydro-2H-pyrazolo[4,3-b]pyridin-2-yl)acetonitrile (250 mg, 0.5 mmol) in CH₃CN (5 mL) was added Selectfluor fluorinating reagent (198 mg, 0.55 mmol). The mixture was stirred at room temperature for 16 hrs. The reaction was quenched with water, extracted with EA (80 mL), washed with brine, dried over Na₂SO₄, filtered, and concentrated to dryness. The resulting residue was purified by Prep-HPLC (Method A) to give the titled Compound A48 (85 mg, 32%), which was further separated into Compound A48a (22 mg) and Compound A48b (35 mg) by chiral-Prep-HPLC. The chiral separation conditions are shown below.

| Column | CHIRALPAK IC |
|---|---|
| Column Size | 2 cm × 25 cm, 5 um |
| Mobile Phase A | MTBE |
| Mobile Phase B | ETOH |
| Flow Rate | 20 mL/min |
| Wave Length | UV 220 nm |
| Temperature | 25° C. |
| Prep-HPLC Equipment | Prep-HPLC-Gilson |

Compound A48a (the earlier peak): ¹H NMR (500 MHz, CD₃OD) b 8.30 (d, J=1.5 Hz, 1H), 7.96 (s, 1H), 7.71 (dd, J=1.5 Hz, 8.0 Hz, 1H), 7.20 (d, J=8.5 Hz, 1H), 5.47 (s, 2H), 4.60-4.52 (m, 1H), 3.92-3.75 (m, 2H), 3.65-3.58 (m, 1H), 3.49 (s, 3H), 3.07-3.00 (m, 1H), 2.96-2.89 (m, 1H), 2.34-2.28 (m, 1H), 2.15-2.05 (m, 2H), 1.98-1.88 (m, 1H), 1.70-1.45 (m, 2H), 1.34 (d, J=6.5 Hz, 3H), 1.06-1.00 (m, 2H), 0.96-0.91 (m, 2H), 0.87 (t, 0.1=7.5 Hz, 3H) 0.59 (t, J=7.0 Hz, 3H) ppm. MS: M/e 492 (M+1)⁺.

Compound A48b (the later peak): ¹H NMR (500 MHz, CD₃OD) δ 8.33 (d, J=1.5 Hz, 1H), 7.97 (s, 1H), 7.72 (dd, J=2.0 Hz, 8.0 Hz, 1H), 7.18 (d, J=8.0 Hz, 1H), 5.46 (s, 2H), 4.38-4.29 (m, 1H), 4.17-4.10 (m, 1H), 3.85-3.78 (m, 1H), 3.68-3.60 (m, 1H), 3.49 (s, 3H), 3.11-3.04 (m, 1H), 2.87-2.80 (m, 1H), 2.27-2.21 (m, 1H), 2.12-2.04 (m, 1H), 1.99-1.88 (m, 1H), 1.80-1.68 (m, 2H), 1.59-1.48 (m, 1H), 1.31 (d, J=6.5 Hz, 3H), 1.04-0.98 (m, 2H), 0.96-0.91 (m, 2H), 0.88 (t, J=7.5 Hz, 3H) 0.62 (t, J=7.5 Hz, 3H) ppm. MS: M/e 492 (M+1)⁺.

### Compound A49: 2-(7-((2S,5R)₄-(1-(2-cyclopropylpyrimidin-5-yl)ethyl)-2,5-diethylpiperazin-1-yl)-4-methyl-5-oxo-4,5-dihydro-2H-pyrazolo[4,3-b]pyridin-2-yl)acetonitrile

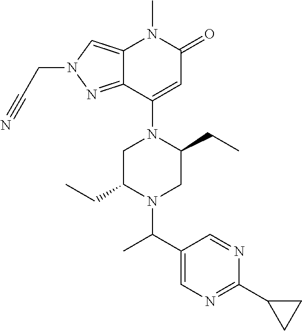 [SMILES: CC[C@H]1CN(C(C)c2cnc(C3CC3)nc2)[C@H](CC)CN1c1cc(=O)n(C)c2cn(CC#N)nc12]

### Step A: 2-cyclopropyl-N-methoxy-N-methylpyrimidine-5-carboxamide

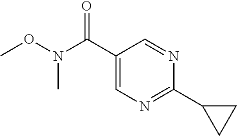

A mixture of 2-cyclopropylpyrimidine-5-carboxylic acid (328 mg, 2 mmol), N,O-dimethylhydroxylamine hydrochloride (234 mg, 2,4 mmol), HATU (917 mg, 2,4 mmol) and DIPEA (516 mg, 4 mmol) in CH₂Cl₂ (15 mL) was stirred overnight. The reaction mixture was washed with H₂O, brine, extracted with CH₂Cl₂ (15 mL×2). The combined organic layers were washed with brine, dried over Na₂SO₄, concentrated to dryness. The resulting residue was purified by flash column chromatography to give the titled compound (427 mg, 100%). MS: M/e 208 (M+1)⁺.

### Step B: 1-(2-cyclopropylpyrimidin-5-yl)ethan-1-one

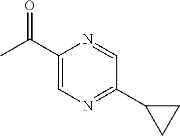

To a stirred solution of 2-cyclopropyl-N-methoxy-N-methylpyrimidine-5-carboxamide (427 mg, 2 mmol) in THF (10 mL) was added dropwise MeMgBr (3.0 M, 1 mL, 3 mmol) at 0° C. After the addition, the reaction mixture was stirred for 30 min. The reaction was quenched with aq·NH₄Cl, extracted with EtOAc (15 mL×3). The combined organic layers were washed with brine, dried over Na₂SO₄, concentrated to dryness. The resulting residue was purified by flash column chromatography to give the titled compound (204 mg, 63%). MS: M/e 163 (M+1)⁺.

### Step C: 1-(2-cyclopropylpyrimidin-5-yl)ethan-1-ol

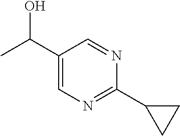

To a stirred solution of 1-(2-cyclopropylpyrimidin-5-yl)ethan-1-one (204 mg, 1,26 mmol) in MeOH (5 mL) was added NaBH₄ (48 mg, 1,26 mmol). After then, the reaction mixture was stirred for 10 min. The reaction mixture was quenched with H₂O, then most MeOH was removed to give the aqueous layer, then extracted with EtOAc (10 mL×2). The combined organic layers were washed with brine, dried over Na₂SO₄, concentrated to give the titled compound (200 mg, 96.7%). MS: M/e 165 (M+1)⁺.

### Step D: 2-(7-((2S,5R)-4-(1-(2-cyclopropylpyrimidin-5-yl)ethyl-2,5-diethylpiperazin-1-yl-4-methyl-5-oxo-4,5-dihydro-2H-pyrazolo[4,3-b]pyridin-2-yl)acetonitrile

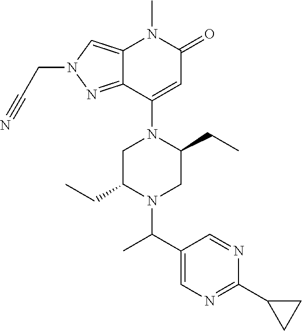 [SMILES: CC[C@H]1CN(C(C)c2cnc(C3CC3)nc2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), 1-(2-cyclopropylpyrimidin-5-yl)ethan-1-ol (32.8 mg, 0.2 mmol), (cyanomethyl)trimethylphosphonium iodide (72.9 mg, 0.3 mmol) and DIPEA (64.5 mg, 0.5 mmol) in CHCN (4 mL) was stirred overnight at 100° C. in a sealed tube. The reaction mixture was diluted with EtOAc (10 mL), then washed with brine, dried over Na₂SO₄, concentrated to dryness. The resulting residue was purified by Prep-HPLC (Method B) to give Compound A49a (5 mg) and Compound A49b (6 mg).

Compound A49a (the earlier peak): ¹H NMR (500 MHz, CD₃OD) δ 8.60 (s, 2H), 7.92 (s, 1H), 5.56 (s, 1H), 5.47 (s, 2H), 3.91-3.79 (m, 1H), 3.43 (s, 3H), 3.35-3.29 (m, 3H), 2.98-2.87 (m, 2H), 2,45-2.35 (m, 1H), 2.28-2.17 (m, 1H), 2.11-1.98 (m, 1H), 1.87-1.73 (m, 1H), 1.66-1.50 (m, 2H), 1.40 (d, J=6.4 Hz, 3H), 1.15-1.06 (m, 4H), 0.93 (t, J=7.2 Hz, 3H), 0.77 (t, J=7.2 Hz, 3H) ppm. MS: M/e 475 (M+1)⁺.

Compound A49b (the later peak): ¹H NMR (500 MHz, CD₃OD) δ 8.62 (s, 2H), 7.93 (s, 1H), 5.56 (s, 1H), 5.47 (s, 2H), 3.72-3.66 (m, 1H), 3.54-3.47 (m, 1H), 3.43 (s, 3H), 3.33-3. 25(m, 2H), 3.21-3.10 (m, 1H), 2.76-2.66 (m, 1H), 2.34-2.15 (m, 2H), 1.96-1.81 (m, 1H), 1.77-1.48 (m, 3H), 1.36 (d, J=6.4 Hz, 3H), 1.12-1.06 (m, 4H), 1.02 (t, J=7.2 Hz, 3H), 0.65 (t, J=7.2 Hz, 3H) ppm. MS: M/e 475 (M+1)⁺.

### Compound A50: 2-(7-((2S,5R)-2,5-diethyl-4-(1-(6-methyl-5-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile

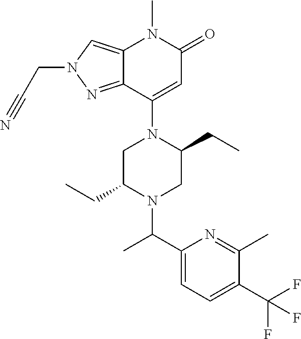 [SMILES: CC[C@H]1CN(C(C)c2ccc(C(F)(F)F)c(C)n2)[C@H](CC)CN1c1cc(=O)n(C)c2cn(CC#N)nc12]

### Step A: 2-methyl-3-(trifluoromethyl)pyridine

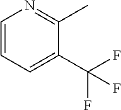

To a mixed solution of 2-chloro-3-(trifluoromethyl)pyridine (6.0 g, 33.1 mmol) and ferric acetylacetonate (1,2 g, 3.3 mmol) in hexane (60 mL) was added MeMgBr (1.4 M, 40 mL, 56 mmol) in drops at −25° C. under N₂. After the addition finished, the mixture was allowed warm to room temperature and stirred for 2 hours. The mixture was quenched with NH₄Cl aqueous solution (100 mL). And the resulted suspension was filtered through a celite pad. The pad was washed with CH₂Cl₂ (100 mL). The filtrates were combined and the layers were separated. The organic layer was washed with brine (100 mL×2), dried over Na₂SO₄, filtered and the filtrate was used for the next step directly without any purification. (150 mL, a crude solution). MS: M/e 162 (M+1)⁺.

### Step B: 2-methyl-3-(trifluoromethyl)pyridine 1-oxide

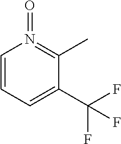

To a stirred solution of 2-methyl-3-(trifluoromethyl)pyridine (150 mL, 33.1 mmol max.) in CH₂Cl₂ (100 mL) was added m-CPBA (15.2 g, 66.2 mmol) in portions at room temperature and the mixture was stirred at room temperature for 24 hours. The resulted suspension was filtered and the filtrate was washed with NaHCO₃(aq., 100 mL×2), dried over Na₂SO₄, filtered and concentrated to dryness. The resulted residue was purified by column chromatography to give the titled compound (4.3 g, 73% for 2 steps). ¹H NMR (400 MHz, CDCl₃) δ 8.44 (d, J=6.4 Hz, 1H), 7.52 (d, J=8.0 Hz, 1H), 7.32-7.23 (m, 1H), 2.66 (s, 3H). MS: M/e 178 (M+1)⁺.

### Step C: 6-methyl-5-(trifluoromethyl)picolinonitrile

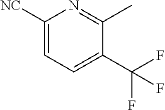

A mixture of 2-methyl-3-(trifluoromethyl)pyridine 1-oxide (1.77 g, 10 mmol), trimethylsilanecarbonitrile (1.50 g, 15 mmol), dimethylcarbamic chloride (1.61 g, 15 mmol) and DIPEA (1.30 g, 10 mmol) in MeCN (20 mL) was stirred at 70° C. for 3 hours. The mixture was cooled and treated with NaHCO₃ (40 mL), extracted with EtOAc (30 mL×2). The extracts were combined and washed with brine (30 mL×2), dried over Na₂SO₄, filtered and concentrated to dryness. The resulted residue was purified by column chromatography to give the titled compound (545 mg, 29%). ¹H NMR (400 MHz, CDCl₃) δ 8.05 (d, J=8.0 Hz, 1H), 7.66 (d, J=8.0 Hz, 1H), 2.77 (s, 3H). MS: M/e 187 (M+1)⁺.

### Step D: 1-(6-methyl-5-(trifluoromethyl)pyridin-2-yl)ethan-1-one

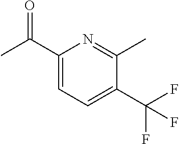

To a 0° C. solution of 6-methyl-5-(trifluoromethyl)picolinonitrile (350 mg, 1.88 mmol) in THF (4 mL) was added MeMgBr (3.0 M, 1,25 mL, 3.75 mmol) in drops under N₂. The mixture was stirred at room temperature for 2 hours. Aqueous solution of NH₄Cl (15 mL) was added and the mixture was extracted with EtOAc (15 mL×2), the organics were combined and washed with brine (15 mL×2), dried over Na₂SO₄, filtered and concentrated to dryness to give the titled compound (165 mg, 43%). ¹H NMR (500 MHz, CDCl₃) δ 8.03 (d, J=8.0 Hz, 1H), 7.94 (d, J=8.0 Hz, 1H), 2.78 (s, 3H), 2.74 (s, 3H). MS: M/e 204 (M+1)⁺.

### Step E: 1-(6-methyl-5-(trifluoromethyl)pyridin-2-yl)ethan-1-ol

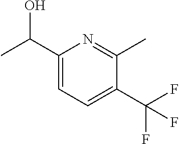

To a solution of 1-(6-methyl-5-(trifluoromethyl)pyridin-2-yl)ethan-1-one (165 mg, 0.8 mmol) in MeOH (4 mL) was added NaBH₄ (31 mg, 0.8 mmol) at room temperature and the mixture was stirred at RT for 2 hours. The mixture was diluted with EA (20 mL), washed with brine (10 mL×2), dried over Na₂SO₄, filtered and concentrated to dryness to give the titled compound (133 mg, 81%). ¹H NMR (500 MHz, DMSO-d₆) δ 8.08 (d, J=8.5 Hz, 1H), 7.53 (d, J=8.5 Hz, 1H), 5.52 (d, J=4.5 Hz, 1H), 4.80-4.67 (m, 1H), 2.61 (s, 3H), 1.37 (d, J=6.5 Hz, 3H). MS: M/e 206 (M+1)⁺.

### Step F: 2-(7-(2S,5R)-2,5-diethyl-4-(1-(6-methyl-5-(trifluoromethyl)pyridin-2-yl)ethyl)piperazin-1-yl-4-methyl-5-oxo-4,5-dihydro-2H-pyrazolo[4,3-b]pyridin-2-yl)acetonitrile

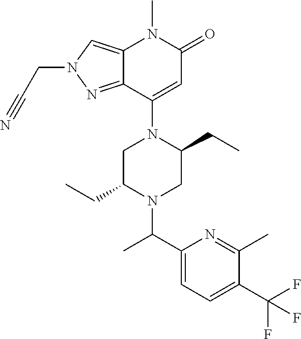 [SMILES: CC[C@H]1CN(C(C)c2ccc(C(F)(F)F)c(C)n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), 1-(6-methyl-5-(trifluoromethyl)pyridin-2-yl)ethan-1-ol (41 mg, 0.2 mmol) and (cyanomethyl)trimethylphosphonium iodide (85 mg, 0.35 mmol) in CH₃CN (1 mL) was added DIPEA (85 mg, 0.66 mmol). The mixture was stirred at 100° C. in a sealed tube for 16 hours. The mixture was cooled and diluted with EtOAc (10 mL), washed with brine (5 mL×3), dried over Na₂SO₄, filtered and concentrated under reduced pressure. The resulting residue was purified by flash column chromatography to give the titled compound (31 mg, 60%). ¹H NMR (500 MHz, CD₃OD) δ 8.12-7.99 (m, 1H), 7.97-7.88 (m, 1H), 7.71-7.52 (m, 1H), 5.57 (s, 1H), 5.49-5.43 (m, 2H), 4.85-4.14 (m, 2H), 4.05-3.75 (m, 1H), 3.58-3.33 (m, 4H), 3.19-2.74 (m, 2H), 2.68 (s, 3H), 2,46-2.20 (m, 1H), 2.15-1.91 (m, 1H), 1.88-1.46 (m, 3H), 1.43-1.31 (m, 3H), 1.08-0.91 (m, 3H), 0.80-0.63 (m, 3H) ppm. MS: M/e 516 (M+1)⁺.

### Compound A51: 2-(7-((2S,5R)-2,5-diethyl-4-(1-(5-isopropoxypyridin-2-yl)ethyl)piperazin-1-yl)-4-methyl-5-oxo-4,5-dihydro-2H-pyrazolo[4,3-b]pyridin-2-yl)acetonitrile

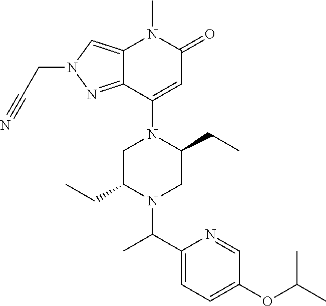 [SMILES: CC[C@H]1CN(C(C)c2ccc(OC(C)C)cn2)[C@H](CC)CN1c1cc(=O)n(C)c2cn(CC#N)nc12]

### Step A: 1-(5-isopropoxypyridin-2-yl)ethan-1-one

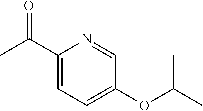

To a solution of 1-(5-hydroxypyridin-2-yl)ethan-1-one (1.37 g, 10 mmol) and K₂CO₃ (2.74 g, 20 mmol) in CH₃CN (10 mL) was added 2-iodopropane (3.4 g, 20 mmol). The mixture was heated at 95° C. for 16 hours under N₂. The reaction mixture was cooled to room temperature, extracted with EA (80 mL×2), washed with brine, dried over Na₂SO₄, filtered, and concentrated to give titled compound (1.62 g, crude). MS: M/e 180 (M+1)⁺.

### Step B: 1-(5-isopropoxypyridin-2-yl)ethan-1-ol

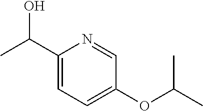

To a solution of 1-(5-isopropoxypyridin-2-yl)ethan-1-one (1.62 g, 9 mmol) in MeOH (20 mL) at ice-cooled bath was added NaBH₄ (380 mg, 10 mmol) in some portions. The mixture was stirred at room temperature for 2 hrs. The reaction was quenched with cold water, extracted with EA (80 mL×2), washed with brine, dried over Na₂SO₄, filtered, and concentrated to dryness. The resulting residue was purified by flash column chromatography to give the titled compound (190 mg, 55% for two steps). MS: M/e 182 (M+1)⁺.

### Step C: 2-(7-((2S,5R)-2,5-diethyl-4-(1-(5-isopropoxypyridin-2-yl)ethyl)piperazin-1-yl)-4-methyl-5-oxo-4,5-dihydro-2H-pyrazolo[4,3-b]pyridin-2-yl)acetonitrile

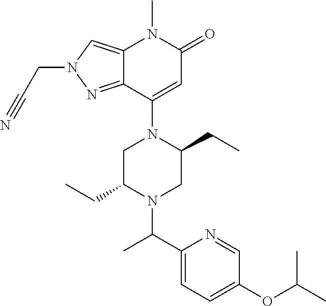 [SMILES: CC[C@H]1CN(C(C)c2ccc(OC(C)C)cn2)[C@H](CC)CN1c1cc(=O)n(C)c2cn(CC#N)nc12]

To a solution of 1-(5-isopropoxypyridin-2-yl)ethan-1-ol (720 mg, 4 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (660 mg, 2 mmol) and (cyanomethyl)trimethylphosphonium iodide (1.46 g, 6 mmol) in CH₃CN (20 mL) was added DIPEA (1.3 g, 10 mmol). The mixture was sealed in a bottle and heated at 100° C. for 16 hours. Then the mixture was cooled to room temperature, diluted with water, extracted with EA (80 mL×2), washed with brine, dried over Na₂SO₄, filtered, and concentrated to dryness. The resulting residue was purified by Prep-TLC (DCM/MeOH=20/1) to give the titled Compound A51, which was further separated into Compound A51a (96 mg) and Compound A51b (133 mg) by twice Prep-HPLC (Method A and Method B).

Compound A51: ¹H NMR (400 MHz, CD₃OD) δ 8.14-8.06 (m, 1H), 7.95-7.91 (m, 1H), 7.58-7.39 (m, 2H), 5.55 (s, 1H), 5.50-5.44 (m, 2H), 4.75-4.58 (m, 1H), 3.94-3.81 (m, 0.5H), 3.75-3.66 (m, 0.5H), 3.55-3.46 (m, 0.5H), 3.43 (s, 3H), 3.31-3.26 (m, 2,5H), 3.20-3.10 (m, 0.5H), 3.06-2.98 (m, 0.5H), 2.92-2.83 (m, 0.5K), 2.72-2.63 (m, 0.5H), 2,41-2.25 (m, 1H), 2.19-1.46 (m, 4H), 1.41-1,27 (m, 9H), 1.02 (t, J=7.6 Hz, 1.5H), 0.95 (t, J=7.6 Hz, 1.5H), 0.71 (t, J=7.6 Hz, 1.5K), 0.65 (t, J=7.6 Hz, 1.5H) ppm. MS: M/e 492 (M+1)⁺.

Compound A51a (the earlier peak): ¹H NMR (500 MHz, CD₃OD) δ 8.09 (d, J=2,5 Hz, 1H), 7.92 (s, 1H), 7.49 (d, J=8.5 Hz, 1H), 7.42 (dd, J=3.0 Hz, 8.5 Hz, 1H), 5.55 (s, 1H), 5.46 (s, 2H), 4.72-4.62 (m, 1H), 3.90-3.83 (m, 1H), 3.43 (s, 3H), 3.36-3.32 (m, 1H), 3.31-3.27 (m, 2H), 3.02-2.85 (m, 2H), 2.39-2.31 (m, 1H), 2.15-2.02 (m, 1H), 1.86-1.76 (m, 1H), 1.60-1.47 (m, 2H), 1.38-1.32 (m, 9H), 0.95 (t, 0.1=7.5 Hz, 3H), 0.71 (t, J=7.6 Hz, 3H) ppm. MS: M/e 492 (M+1)⁺.

Compound A51b (the later peak): ¹H NMR (500 MHz, CD₃OD) δ 8.08 (d, J=3.0 Hz, 1H), 7.92 (s, 1H), 7.54 (d, J=8.5 Hz, 1H), 7.41 (dd, J=3.0 Hz, 8.5 Hz, 1H), 5.55 (s, 1H), 5.47 (s, 2H), 4.71-4.62 (m, 1H), 3.74-3.65 (m, 1H), 3.54-3.46 (m, 1H), 3.43 (s, 3H), 3.31-3.26 (m, 2H), 3.18-3.10 (m, 1H), 2.76-2.65 (m, 1H), 2.29-2.22 (m, 1H), 2.02-1.90 (m, 1H), 1.75-1.45 (m, 3H), 1.38-1.30 (m, 9H), 1.02 (t, J=7.5 Hz, 3H), 0.64 (t, J=7.6 Hz, 3H) ppm. MS: M/e 492 (M+1)⁺.

### Compound A52: 2-(7-((2S,5R)-4-(1-(5-cyclopropylpyridin-2-yl)ethyl)-2,5-diethylpiperazin-1-yl)-4-methyl-5-oxo-4,5-dihydro-2H-pyrazolo[4,3-b]pyridin-2-yl)acetonitrile

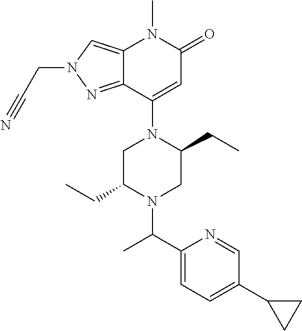 [SMILES: CC[C@H]1CN(C(C)c2ccc(C3CC3)cn2)[C@H](CC)CN1c1cc(=O)n(C)c2cn(CC#N)nc12]

### Step A: 1-(5-cyclopropylpyridin-2-yl)ethan-1-one

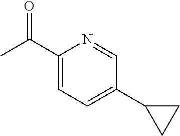

To a solution of 1-(5-bromopyridin-2-yl)ethan-1-one (200 mg, 1 mmol), cyclopropylboronic acid (94.6 mg, 1.1 mmol), potassium phosphate (318 mg, 1.5 mmol) and tricyclohexylphosphine (28 mg, 0.1 mmol) in toluene (5 mL) and water (0.5 ml) under a nitrogen atmosphere was added palladium acetate (22,4 mg, 0.1 mmol). The mixture was heated to 100° C. overnight and then cooled to room temperature. Water (50 mL) was added and the mixture extracted with EtOAc (3×10 mL), the combined organics were washed with brine (50 mL), dried over Na₂SO₄ and concentrated in vacuo. The resulting residue was purified by flash column chromatography (EtOAc: PE=0-50% in 20 minutes) to give the titled compound (100 mg, 62%). MS: M/e 162 (M+1)⁺.

### Step B: 1-(5-cyclopropylpyridin-2-yl)ethan-1-ol

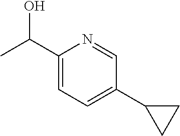

To a solution of 1-(5-cyclopropylpyridin-2-yl)ethan-1-one (100 mg, 0.62 mmol) in MeOH (3 mL) was added NaBH₄ (19 mg, 0.5 mmol) at room temperature and the mixture was stirred at room temperature for 30 minutes. The resulting mixture was treated with water(30 mL), extracted with DCM (10 mL×2). The combined organic layers were washed with brine (20 mL×1), dried over Na₂SO₄ and concentrated to dryness. The resulting residue (100 mg, crude) was used in the next step. MS: M/e 164 (M+1)⁺.

### Step C: 2-(7-((2S,5R)-4-(1-(5-cyclopropylpyridin-2-yl)ethyl-2,5-diethylpiperazin-1-yl)-4-methyl-5-oxo-4,5-dihydro-2H-pyrazolo[4,3-b]pyridin-2-yl)acetonitrile

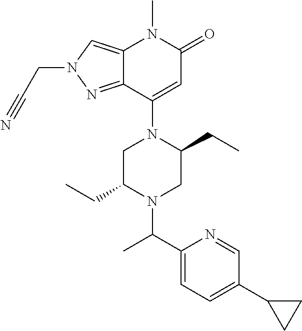 [SMILES: CC[C@H]1CN(C(C)c2ccc(C3CC3)cn2)[C@H](CC)CN1c1cc(=O)n(C)c2cn(CC#N)nc12]

A mixture of 1-(5-cyclopropylpyridin-2-yl)ethan-1-ol (25 mg, 0.15 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (48 mg, 0.2 mmol) and DIPEA (51 mg, 0.4 mmol) in MeCN (2 mL) was stirred at 100° C. overnight. The reaction mixture was diluted with EtOAc (10 mL), washed with brine (10 mL×2), dried over Na₂SO₄ and concentrated to dryness. The resulting residue was purified by flash column chromatography (MeOH: DCM=0-10% in 20 minutes) to give the titled compound (28 mg, 59%). ¹H NMR (500 MHz, DMSO-d₆) δ 8.35-8.23 (m, 1H), 7.97 (d, J=2.7 Hz, 1H), 7.44-7.30 (m, 2H), 5.61 (d, J=3.1 Hz, 2H), 5.38 (s, 1H), 3.73 (dq, J=19.5, 6.3 Hz, 1H), 3.37 (s, 2H), 3.27 (s, 3H), 3.18-2.98 (m, 1H), 2.90-2.77 (m, 1H), 2,58 (d, J=8.9 Hz, 1H), 2.33-2.17 (m, 1H), 1.99-1.77 (m, 2H), 1.63-1.36 (m, 3H), 1.30-1,20 (m, 3H), 1.03-0.98 (m, 4H), 0.86-0.68 m, 4H), 0.66-0.52 (m, 3H) ppm. MS: M/e 474 (M+1)⁺.

### Compound A53: 2-(7-((2S,5R)-4-(1-(3,5-dimethylpyridin-2-yl)ethyl)-2,5-diethylpiperazin-1-yl)-4-methyl-5-oxo-4,5-dihydro-2H-pyrazolo[4,3-b]pyridin-2-yl)acetonitrile

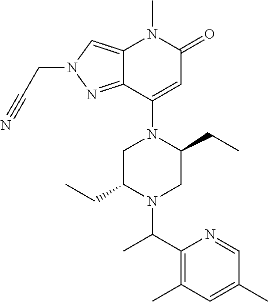 [SMILES: CC[C@H]1CN(C(C)c2ncc(C)cc2C)[C@H](CC)CN1c1cc(=O)n(C)c2cn(CC#N)nc12]

### Step A: 1-(3.5-dimethylpyridin-2-yl)ethan-1-one

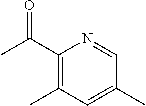

A mixture of 2-bromo-3,5-dimethylpyridine(372 mg, 2 mmol), tributyl(1-ethoxyvinyl)stannane (1.08 g, 3 mmol) and Pd(PPh₃)₂Cl₂ (140 mg, 0.2 mmol) in toluene (5 mL) was stirred at 100° C. under N₂ for 16 hours. To the resulting solution was added HCl (0.5 mL, con.) in drops and the mixture was stirred at room temperature for 30 minutes. The mixture was treated with saturated NaHCO₃ aq. (30 ml), extracted with EtOAc(10 ml×3), washed with brine (20 mL), dried and concentrated to dryness. The resulting residue was purified by flash column chromatography (EtOAc: PE=0-50% in 20 minutes) to give the titled compound (80 mg, 27%). MS: M/e 150 (M+1)⁺.

### Step B: 1-(3.5-dimethylpyridin-2-yl)ethan-1-ol

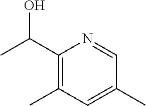

To a solution of 1-(3,5-dimethylpyridin-2-yl)ethan-1-one (80 mg, 0.54 mmol) in MeOH (3 mL) was added NaBH₄ (16 mg, 0.43 mmol) at room temperature and the mixture was stirred at room temperature for 30 minutes. The resulting mixture was treated with water(50 ml), extracted with DCM (20 mL×2). The combined organic layers were dried over Na₂SO₄ and concentrated to dryness. The resulting residue (70 mg, crude) was used in the next step. MS: M/e 152 (M+1)⁺.

### Step C: 2-(7-((2S,5R)-4-(-(3.5-dimethylpyridin-2-yl)ethyl)-2,5-diethylpiperazin-1-yl)-4-methyl-5-oxo-4,5-dihydro-2H-pyrazolo[4,3-b]pyridin-2-yl)acetonitrile

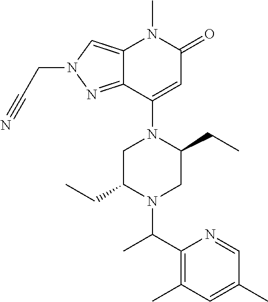 [SMILES: CC[C@H]1CN(C(C)c2ncc(C)cc2C)[C@H](CC)CN1c1cc(=O)n(C)c2cn(CC#N)nc12]

A mixture of 1-(3,5-dimethylpyridin-2-yl)ethan-1-ol (23 mg, 0.15 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (48 mg, 0.2 mmol) and DIPEA (51 mg, 0.4 mmol) in MeCN (2 mL) was stirred at 100° C. overnight. The reaction mixture was diluted with EtOAc (20 mL), washed with water (10 mL×2), dried over Na₂SO₄ and concentrated to dryness. The resulting residue was purified by flash column chromatography (MeOH:DCM=0-10% in 20 minutes) to give the titled compound (16 mg, 35%). ¹H NMR (500 MHz, DMSO-d,) 6 8.17 (d, J=13.0 Hz, 1H), 7.97 (d, J=1.5 Hz, 1H), 7.36 (d, J=7.3 Hz, 1H), 5.62 (dd, .=17.0, 6.6 Hz, 2H), 5.37 (t, J=16.5 Hz, 1H), 4.07 (dt, J=24.2, 6.7 Hz, 1H), 3.32 (s, 1H), 3.31-3.16 (m, 4H), 2.95-2.80 (m, 1H), 2.78-2.66 (m, 1H), 2,40 (s, 3H), 2.37-2.21 (m, 4H), 1.82-1.65 (m, 1H), 1.60-1,22 (m, 7H), 0.92-0.85 (m, 1.5H), 0.68-0.53 (m, 4.5H) ppm. MS: M/e 462 (M+1)⁺.

### Compound A54: 2-(7-((2S,5R)-4-(1-(5,6-dimethylpyridin-2-yl)ethyl)-2,5-diethylpiperazin-1-yl)-4-methyl-5-oxo-4,5-dihydro-2H-pyrazolo[4,3-b]pyridin-2-yl)acetonitrile

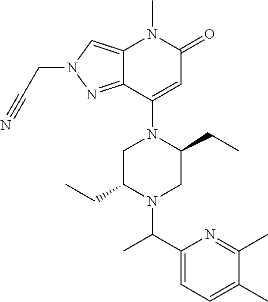 [SMILES: CC[C@H]1CN(C(C)c2ccc(C)c(C)n2)[C@H](CC)CN1c1cc(=O)n(C)c2cn(CC#N)nc12]

### Step A: 1-(5,6-dimethylpyridin-2-yl)ethan-1-one

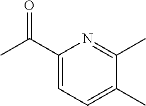

A mixture of 6-bromo-2,3-dimethylpyridine (400 mg, 2.1 mmol), tributyl(1-ethoxyvinyl)stannane (1.5 g, 4.2 mmol) and Pd(PPh₃)₂Cl₂ (151 mg, 0.21 mmol) in toluene (10 mL) was stirred at 100° C. under N₂ for 16 hours. The reaction mixture was diluted with HCl (2 mL, 3 M in ethyl acetate) in drops and the mixture was stirred at room temperature for 0.5 hour. The reaction was concentrated and adjusted to pH=8-9 with saturated NaHCO₃ solution, extracted with DCM (20 mL×2). The combined organic layers were washed with brine, dried over Na₂SO₄ and concentrated to dryness. The resulting residue was purified by flash column chromatography to give the titled compound (120 mg, 37%). MS: M/e 150 (M+1)⁺

### Step B: 1-(5,6-dimethylpyridin-2-yl)ethan-1-ol

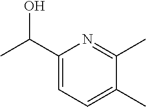

To a solution of 1-(5,6-dimethylpyridin-2-yl)ethan-1-one (120 mg, 0.8 mmol) in MeOH (3 mL) was added NaBH₄ (18 mg, 0.48 mmol) at room temperature and the resulting mixture was stirred at room temperature for 5 min. The reaction mixture was diluted with DCM and washed with water, dried over Na₂SO₄ and concentrated to give the titled compound (120 mg). MS: M/e 152 (M+1)⁺.

### Step C: 2-(7-((2S,5R)-2,5-diethyl-4-(1-(1-methylisoquinolin-6-yl)ethyl)piperazin-1-yl)-4-methyl-5-oxo-4,5-dihydro-2H-pyrazolo[4,3-b]pyridin-2-yl)acetonitrile

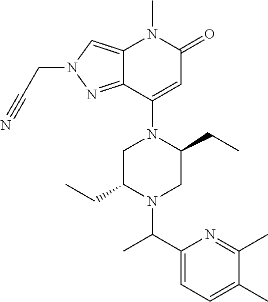 [SMILES: CC[C@H]1CN(C(C)c2ccc(C)c(C)n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (2 mL) and was added 1-(5,6-dimethylpyridin-2-yl)ethan-1-ol (69 mg, 0.45 mmol), (cyanomethyl)trimethylphosphonium iodide (111 mg, 0.45 mmol) and DIPEA(197 mg, 1.5 mmol).The resulting mixture was stirred at 105° C. overnight. The reaction solvent was removed under reduced pressure. The resulting residue was purified by flash column chromatography to give the titled compound (crude), which was further purified to give the titled Compound A54a (14 mg) and Compound A54b (14 mg) by Prep-chiral HPLC. The chiral separation conditions are shown below.

| Column | IE |
|---|---|
| Column Size | 20.0 mm × 250 mm, 5 um |
| Mobile Phase A | HEXANE 60% |
| Mobile Phase B | ETOH(0.2% 2M NH₃MEOH) 40% |
| Run Time | 13 min |
| Flow Rate | 18 mL/min |
| Wave Length | UV 210 nm and 310 nm |
| Temperature | RT |
| Prep-HPLC Equipment | Prep-HPLC-Gilson |

Compound A54a (the earlier peak): ¹H NMR (400 MHz, CD₃OD) δ 7.92 (s, 1H), 7.56 (d, J=7.8 Hz, 1H), 7.34 (d, J=7.8 Hz, 1H), 5.56 (s, 1H), 5.46 (s, 2H), 4.85-4.02 (m, 2H), 3.88 (q, J=6.6 Hz, 1H), 3.44 (d, J=5.6 Hz, 3H), 3.33 (s, 1H), 3.01 (d, J=10.7 Hz, 1H), 2.90 (dd, J=12.1, 3.8 Hz, 1H), 2,49 (s, 3H), 2.38 (d, J=9.6 Hz, 1H), 2.30 (s, 3H), 2.19-2.03 (m, 1H), 1.90-1.73 (m, 1H), 1.66-1.49 (m, 2H), 1.32 (t, J=9.0 Hz, 3H), 0.96 (t, J=7.4 Hz, 3H), 0.70 (t, J=7.4 Hz, 3H) ppm. MS: M/e 462 (M+1)⁺.

Compound A54b (the later peak): ¹H NMR (400 MHz, CD₃OD) δ 7.92 (s, 1H), 7.55 (d, J=7.8 Hz, 1H), 7.39 (d, J=7.8 Hz, 1H), 5.56 (s, 1H), 5.47 (s, 2H), 4.82-3.76 (m, 2H), 3.70 (q, J=6.5 Hz, 1H), 3.51 (d, J=12.8 Hz, 1H), 3.44 (d, J=7.0 Hz, 3H), 3.20-3.06 (m, 1H), 2.72 (d, J=9.3 Hz, 1H), 2,48 (s, 3H), 2.30 (s, 3H), 2.27 (d, J=12,4 Hz, 1H), 1.99 (s, 1H), 1.74-1.44 (m, 3H), 1.31 (d, J=6.7 Hz, 3H), 1.02 (t, J=7.3 Hz, 3H), 0.66 (t, J=7.2 Hz, 3H) ppm. MS: M/e 462 (M+1)⁺.

### Compound A55: 2-(7-((2S,5R)-2,5-diethyl-4-(1-(5-methoxy-6-methylpyridin-2-yl)ethyl)piperazin-1-yl)-4-methyl-5-oxo-4,5-dihydro-2H-pyrazolo[4,3-b]pyridin-2-yl)acetonitrile

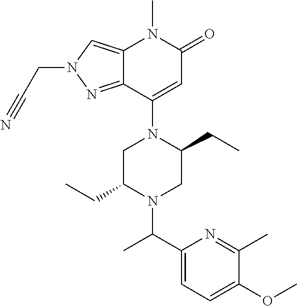 [SMILES: CC[C@H]1CN(C(C)c2ccc(OC)c(C)n2)[C@H](CC)CN1c1cc(=O)n(C)c2cn(CC#N)nc12]

### Step A: 1-(5-methoxy-6-methylpyridin-2-yl)ethan-1-one

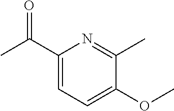

A mixture of 6-bromo-3-methoxy-2-methylpyridine (404 mg, 2 mmol), tributyl(1-ethoxyvinyl)stannane (1.08 g, 3 mmol) and Pd(PPh₃)₂Cl₂ (140 mg, 0.2 mmol) in toluene (5 mL) was stirred at 100° C. under N₂ for 16 hours. To the resulting solution was added HCl (0.5 mL, con.) in drops and the mixture was stirred at room temperature for 30 minutes. The mixture was treated with saturated NaHCO₃ aq. (20 ml), extracted with EtOAc(20 ml×2), washed with brine (20 mL), dried and concentrated to dryness. The resulting residue was purified by flash column chromatography (EtOAc: PE=0-50% in 30 minutes) to give the titled compound (80 mg, 24%). MS: M/e 166 (M+1).

### Step B: 145-methoxy-6-methylpyridin-2-yl)ethan-1-ol

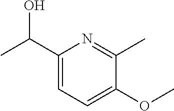

To a solution of 1-(5-methoxy-6-methylpyridin-2-yl)ethan-1-one (80 mg, 0.48 mmol) in MeOH (3 mL) was added NaBH₄ (15 mg, 0.39 mmol) at room temperature and the mixture was stirred at room temperature for 30 minutes. The resulting mixture was treated with water (30 ml), extracted with DCM (10 mL×2). The combined organic layers were dried over Na₂SO₄ and concentrated to dryness. The resulting residue (70 mg, crude) was used in the next step. MS: M/e 168 (M+1)⁺.

### Step C: 2-(7-((2S,5R)-2,5-diethyl-4-(1-(5-methoxy-6-methylpyridin-2-yl)ethyl)piperazin-1-yl-4-methyl-5-oxo-4,5-dihydro-2H-pyrazolo[4,3-b]pyridin-2-yl)acetonitrile

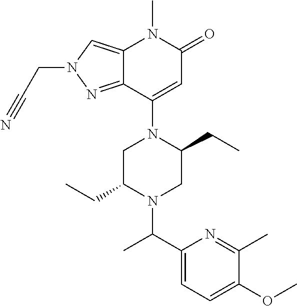 [SMILES: CC[C@H]1CN(C(C)c2ccc(OC)c(C)n2)[C@H](CC)CN1c1cc(=O)n(C)c2cn(CC#N)nc12]

A mixture of 1-(5-methoxy-6-methylpyridin-2-yl)ethan-1-ol (25 mg, 0.15 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (48 mg, 0.2 mmol) and DIPEA (52 mg, 0.4 mmol) in MeCN (2 mL) was stirred at 100° C. overnight. The reaction mixture was diluted with EtOAc (20 mL), washed with water (10 mL×2), dried over Na₂SO₄ and concentrated to dryness. The resulting residue was purified by flash column chromatography (MeOH: DCM=0-10% in 25 minutes) to give the titled compound (13 mg, 27%). ¹H NMR (500 MHz, DMSO-d₆) δ 7.98 (t, J=7.6 Hz, 1H), 7.37-7.20 (m, 2H), 5.62 (t, J=10.2 Hz, 2H), 5.38 (s, 1H), 3.81 (t, J=7.0 Hz, 3H), 3.78-3.58 (m, 1H), 3.36 (d, J=13.1 Hz, 1H), 3.27 (t, J=5.2 Hz, 4H), 3.16-2.97 (m, 1H), 2.88-2,52 (m, 1H), 2,40-2.16 (m, 5H), 2.02-1.80 (m, 1H), 1.64-1.40 (m, 3H), 1,29-1,21 (m, 3H), 0.95-0.83 (m, 3H), 0.61 (dt, J=18.9, 7.1 Hz, 3H) ppm. MS: M/e 478 (M+1)⁺.

### Compound A56: 2-(7-((2S,5R)-2,5-diethyl-4-(1-(6-(methoxymethyl)-5-methylpyridin-2-yl)ethyl)piperazin-1-yl)-4-methyl-5-oxo-4,5-dihydro-2H-pyrazolo[4,3-b]pyridin-2-yl)acetonitrile

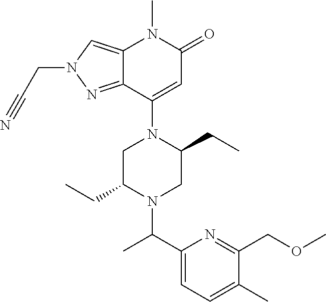 [SMILES: CC[C@H]1CN(C(C)c2ccc(C)c(COC)n2)[C@H](CC)CN1c1cc(=O)n(C)c2cn(CC#N)nc12]

### Step A: 2-(methoxymethyl)-3-methylpyridine

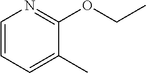

A mixture of 2-(chloromethyl)-3-methylpyridine (1 g, 7 mmol) in MeOH (15 mL) was added NaOMe (1.1 g, 21,2 mol). The reaction was stirred at 50° C. for 16 hours. The solution was dissolved in DCM, washed with water, dried over Na₂SO₄ and concentrated to dryness. The resulting residue purified by flash column chromatography to give the titled compound (600 mg, 61%). MS: M/e 138 (M+1)⁺.

### Step B: 2-(methoxymethyl)-3-methylpyridine 1-oxide

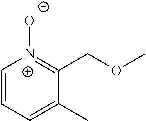

To a solution of 2-(methoxymethyl)-3-methylpyridine (0.6 g, 4.3 mmol) in DCM (20 mL) was added 3-chlorobenzoperoxoic acid (1.5 g, 8.7 mmol). The mixture solution was stirred at room temperature for two days. The reaction was concentrated to dryness. The resulting residue was purified by flash column chromatography to give the titled compound (0.4 g, 60%). MS: M/e 154 (M+1)⁺.

### Step C: 6-methoxymethyl)-5-methylpicolinonitrile

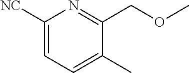

To a solution of 2-(methoxymethyl)-3-methylpyridine 1-oxide (0.4 g, 2.6 mmol) in CH₃CN (5 mL) were added TMSCN (776 mg, 7.8 mmol), Et₃N (792 mg, 7.8 mol). The mixture was stirred at 90° C. for overnight. The reaction was concentrated to dryness. The resulting residue was purified by flash column chromatography to give the titled compound (300 mg, 70%). MS: M/e 163 (M+1)⁺.

### Step D: 1-(6-(methoxymethyl)-5-methylpyridin-2-yl)ethan-1-one

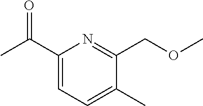

A mixture of 6-(methoxymethyl)-5-methylpicolinonitrile (300 mg, 1.85 mmol) in THF (5 mL) at 0° C. was added methyl magnesium bromide (1,2 mL, 3M, 3.7 mol). The reaction mixture was stirred at room temperature for 1 hour. The reaction was quenched by adding saturated solution of NH₄Cl. The resulting mixture was extracted with EA, and then concentrated by using a rotary evaporator, to give the titled compound (270 mg). MS: M/e 180 (M+1)⁺.

### Step E: 1-(6-(methoxymethyl)-5-methylpyridin-2-yl)ethan-1-ol

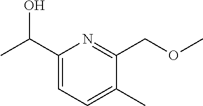

To a solution of 1-(6-(methoxymethyl)-5-methylpyridin-2-yl)ethan-1-one (270 mg, 1.5 mmol) in MeOH (5 mL) was added NaBH₄ (35 mg, 0.9 mmol) at room temperature and the resulting mixture was stirred at room temperature for 5 min. The reaction mixture was diluted with DCM and washed with water, dried over Na₂SO₄ and concentrated to give the titled compound (260 mg). MS: M/e 182 (M+1)⁺.

### Step F: 2-(7-((2S,5R)-2,5-diethyl-4-(1-(6-(methoxymethyl-5-methylpyridin-2-yl)ethyl)piperazin-1-yl)-4-methyl-5-oxo-4,5-dihydro-2H-pyrazolo[4,3-b]pyridin-2-yl)acetonitrile

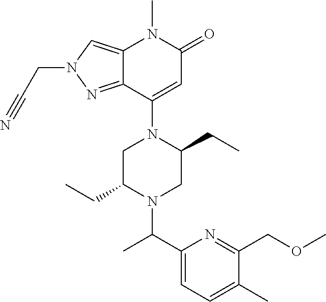 [SMILES: CC[C@H]1CN(C(C)c2ccc(C)c(COC)n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (2 mL) and was added 1-(6-(methoxymethyl)-5-methylpyridin-2-yl)ethan-1-ol (138 mg, 0.76 mmol), (cyanomethyl)trimethylphosphonium iodide (110 mg, 0.45 mmol) and DIPEA(197 mg, 1.5 mmol).The resulting mixture was stirred at 105° C. overnight. The reaction solvent was removed under reduced pressure, The reaction was diluted with DCM and washed with water. The organic layer was separated, dried by Na₂SO₄, filtered, and concentrated to dryness, then purified by flash column chromatography (DCM/MeOH) to give the titled compound(crude), which was further purified to give the titled compound (5 mg) by Prep-HPLC (Method A). ¹H NMR (400 MHz, CD₃OD) δ 7.92 (d, J=3.3 Hz, 1H), 7.68-7.59 (m, 1H), 7.50 (dd, J=27.1, 7.9 Hz, 1H), 5.56 (s, 1H), 5.46 (d, J=4.0 Hz, 2H), 4.60-4.55 (m, 2H), 4.01-3.71 (m, 1H), 3.59-3.48 (m, 1H), 3.42 (dd, J=12.3, 2.8 Hz, 6H), 3.32-3.10 (m, 1H), 3.05-2.71 (m, 2H), 2,47-2.35 (m, 4H), 2.32-2.04 (m, 1H), 2.02-1.49 (m, 4H), 1.34 (dd, J=13.2, 6.7 Hz, 3H), 0.99 (dt, J=34.5, 7.4 Hz, 3H), 0.68 (dt, J=24.2, 7.4 Hz, 3H). ppm. MS: M/e 492 (M+1)⁺.

### Compound A57: 2-(7-((2S,5R)-4-(1-(5-cyclopropyl-3-methylpyridin-2-yl)ethyl)-2,5-diethylpiperazin-1-yl)-4-methyl-5-oxo-4,5-dihydro-2H-pyrazolo[4,3-b]pyridin-2-yl)acetonitrile

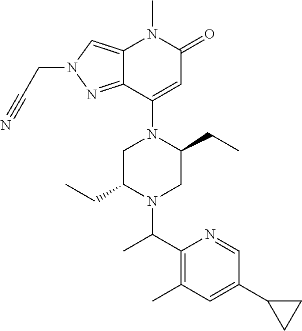 [SMILES: CC[C@H]1CN(C(C)c2ncc(C3CC3)cc2C)[C@H](CC)CN1c1cc(=O)n(C)c2cn(CC#N)nc12]

### Step A: 5-cyclopropyl-3-methylpicolinonitrile

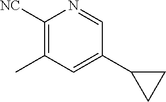

A mixture of 5-bromo-3-methylpicolinonitrile (2.0 g, 10 mmol), cyclopropylboronic acid (1.3 g, 15 mmol), Cs₂CO₃ (6.5 g, 20 mmol), H₂O (2 mL) and Pd(dppf)₂Cl₂ (400 mg, 0.55 mmol) in Dioxane (20 mL) was stirred at 100° C. under N₂ for 16 hours. The mixture was cooled and diluted with EA (30 mL). The resulted mixture was washed with brine (20 mL×3), dried over Na₂SO₄, filtered and concentrated to dryness. The resulting residue was purified by column chromatography to give the titled compound (1.49 g, 94%). ¹H NMR (500 MHz, CDCl₃) δ 8.30 (s, 1H), 7.20 (s, 1H), 2,50 (s, 3H), 1.99-1.87 (m, 1H), 1.19-1.11 (m, 2H), 0.86-0.76 (m, 2H). MS: M/e 159 (M+1)⁺.

### Step B: 1-(5-cyclopropyl-3-methylpyridin-2-yl)ethan-1-one

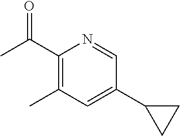

To a 0° C. solution of 5-cyclopropyl-3-methylpicolinonitrile (635 mg, 4.0 mmol) in THF (10 mL) was added MeMgBr (3.0 M, 2.7 mL, 8.0 mmol) in drops under N₂. The mixture was stirred at room temperature for 2 hours. Aqueous solution of NH₄Cl (20 mL) was added and the mixture was extracted with EtOAc (20 mL×2), the organics were combined and dried over Na₂SO₄, filtered and concentrated to dryness. The resulted residue was purified by column chromatography to give the titled compound (580 mg, 83%). ¹H NMR (500 MHz, CDCl₃) δ 8.28 (s, 1H), 7.13 (s, 1H), 2.68 (s, 3H), 2,55 (s, 3H), 1.97-1.87 (m, 1H), 1.16-1.04 (m, 2H), 0.84-0.76 (m, 2H). MS: M/e 176 (M+1)⁺.

### Step C: 1-(5-cyclopropyl-3-methylpyridin-2-yl)ethan-1-ol

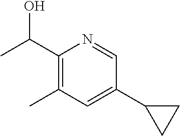

To a solution of 1-(5-cyclopropyl-3-methylpyridin-2-yl)ethan-1-one (580 mg, 3.3 mmol) in MeOH (10 Ml) was added NaBH₄ (75 mg, 2.0 mmol) at room temperature and the mixture was stirred at rt for 2 hours. The mixture was diluted with EA (30 Ml), washed with brine (10 Ml×3), dried over Na₂SO₄, filtered and concentrated to dryness to give the titled compound (530 mg, 90%). ¹H NMR (500 MHz, DMSO-d6) δ 8.17 (s, 1H), 7.17 (s, 1H), 4.95-4.81 (m, 2H), 2.29 (s, 3H), 1.98-1.80 (m, 1H), 1.33 (d, J=6.0 Hz, 3H), 1.01-0.87 (m, 2H), 0.80-0.62 (m, 2H). MS: M/e 178 (M+1)⁺.

### Step D: 2-(7-((2S,5R)-4-(1-(5-cyclopropyl-3-methylpyridin-2-yl)ethyl)-2,5-diethylpiperazin-1-yl-4-methyl-5-oxo-4,5-dihydro-2H-pyrazolo[4,3-b]156yridine-2-yl)acetonitrile

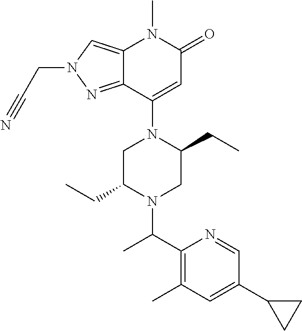 [SMILES: CC[C@H]1CN(C(C)c2ncc(C3CC3)cc2C)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), 1-(5-cyclopropyl-3-methylpyridin-2-yl)ethan-1-ol (36 mg, 0.2 mmol) and (cyanomethyl)trimethylphosphonium iodide (85 mg, 0.35 mmol) in CH₃CN (1 mL) was added DIPEA (85 mg, 0.66 mmol). The mixture was stirred at 100° C. in a sealed tube for 16 hours. The mixture was cooled and diluted with EtOAc (10 mL), washed with brine (5 mL×3), dried over Na₂SO₄, filtered and concentrated under reduced pressure. The resulting residue was purified by prep-TLC (CH₂Cl₂/MeOH=10:1) and Prep-HPLC (Method A) to give the titled compound (16 mg, 33%) as a formic acid salt. ¹H NMR (500 MHz, DMSO-d6) δ 8.20 (s, 1H), 8.18-8.13 (m, 1H), 7.99-7.93 (m, 1H), 7.18-7.12 (m, 1H), 5.66-5.55 (m, 2H), 5.40-5.29 (m, 1H), 5.00-4.30 (m, 1H), 4.12-3.99 (m, 1H), 3.38-3.29 (m, 1H), 3.26 (s, 3H), 3.23-3.15 (m, 1H), 2.97-2.63 (m, 2,5H), 2,42-2.33 (m, 3H), 2.30-2.23 (m, 0.5H), 1.95-1.83 (m, 1H), 1.80-1.59 (m, 1H), 1.58-1.37 (m, 3H), 1.37-1,27 (m, 3H), 1.02-0.92 (m, 2H), 0.89 (t, J=7.5 Hz, 1.5H), 0.74-0.68 (m, 2H), 0.66 (t, J=7.5 Hz, 1.5H), 0.62-0.51 (m, 3H). MS: M/e 488 (M+1)⁺.

### Compound A58: 2-(7-((2S,5R)-4-(1-(5-cyclopropyl-6-methylpyridin-2-yl)ethyl)-2,5-diethylpiperazin-1-yl)-4-methyl-5-oxo-4,5-dihydro-2H-pyrazolo[4,3-b]pyridin-2-yl)acetonitrile

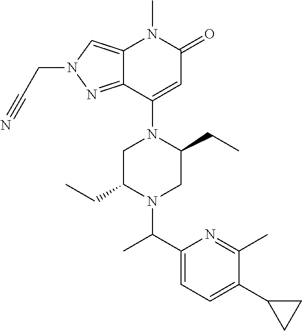 [SMILES: CC[C@H]1CN(C(C)c2ccc(C3CC3)c(C)n2)[C@H](CC)CN1c1cc(=O)n(C)c2cn(CC#N)nc12]

### Step A: 5-cyclopropyl-6-methylnicotinonitrile

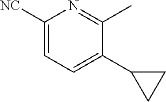

A mixture of 5-bromo-6-methylpicolinonitrile (2.0 g, 10 mmol), cyclopropylboronic acid (1.3 g, 15 mmol), Cs₂CO₃ (6.5 g, 20 mmol), H₂O (2 mL) and Pd(dppf)₂Cl₂ (400 mg, 0.55 mmol) in Dioxane (20 mL) was stirred at 100° C. under N₂ for 16 hours. The mixture was cooled and diluted with EA (30 mL). The resulted mixture was washed with brine (20 mL×3), dried over Na₂SO₄, filtered and concentrated to dryness. The resulted residue was purified by column chromatography to give the titled compound (1.40 g, 88%). ¹H NMR (500 MHz, CDCl₃) δ 7.45 (d, J=7.5 Hz, 1H), 7.29 (d, J=7.5 Hz, 1H), 2.69 (s, 3H), 2.01-1.90 (m, 1H), 1.15-1.07 (m, 2H), 0.74-0.67 (m, 2H). MS: M/e 159 (M+1)⁺.

### Step B: 1-(5-cyclopropyl-6-methylpyridin-2-yl)ethan-1-one

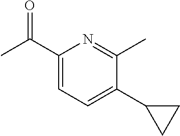

To a 0° C. solution of 5-cyclopropyl-6-methylpicolinonitrile (635 mg, 4.0 mmol) in THF (10 mL) was added MeMgBr (3.0 M, 2.7 mL, 8.0 mmol) in drops under N₂. The mixture was stirred at room temperature for 2 hours. Aqueous solution of NH₄Cl (20 mL) was added and the mixture was extracted with EtOAc (20 mL×2), the organics were combined and dried over Na₂SO₄, filtered and concentrated to dryness. The resulted residue was purified by column chromatography to give the titled compound (530 mg, 76%). ¹H NMR (500 MHz, CDCl₃) δ 7.79 (d, J=8.0 Hz, 1H), 7.30 (d, J=8.0 Hz, 1H), 2.71 (s, 3H), 2.70 (s, 3H), 2.00-1.90 (m, 1H), 1.09-1.03 (m, 2H), 0.74-0.65 (m, 2H). MS: M/e 176 (M+1)⁺.

### Step C: 1-(5-cyclopropyl-6-methylpyridin-2-yl)ethan-1-ol

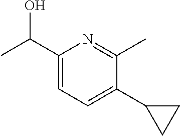

To a solution of 1-(5-cyclopropyl-6-methylpyridin-2-yl)ethan-1-one (530 mg, 3.0 mmol) in MeOH (10 mL) was added NaBH₄ (70 mg, 1.8 mmol) at room temperature and the mixture was stirred at rt for 2 hours. The mixture was diluted with EA (30 mL), washed with brine (10 mL×3), dried over Na₂SO₄, filtered and concentrated to dryness to give the titled compound (510 mg, 96%). ¹H NMR (500 MHz, DMSO-d6) δ 7.29 (d, J=8.0 Hz, 1H), 7.20 (d, J=8.0 Hz, 1H), 5.18 (d, J=5.0 Hz, 1H), 4.68-4.58 (m, 1H), 2,53 (s, 3H), 1.95-1.83 (m, 1H), 1.32 (d, J=6.5 Hz, 3H), 0.98-0.87 (m, 2H), 0.64-0.55 (m, 2H). MS: M/e 178 (M+1)⁺.

### Step D: 2-(7-((2S,5R)-4-(1-(5-cyclopropyl-6-methylpyridin-2-yl)ethyl)-2,5-diethylpiperazin-1-yl-4-methyl-5-oxo-4,5-dihydro-2H-pyrazolo[4,3-b]pyridin-2-yl)acetonitrile

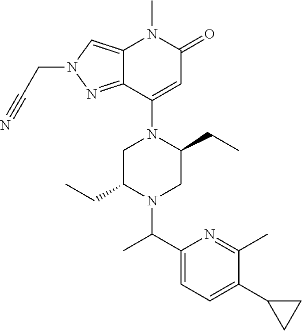 [SMILES: CC[C@H]1CN(C(C)c2ccc(C3CC3)c(C)n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), 1-(5-cyclopropyl-6-methylpyridin-2-yl)ethan-1-ol (36 mg, 0.2 mmol) and (cyanomethyl)trimethylphosphonium iodide (85 mg, 0.35 mmol) in CH₃CN (1 mL) was added DIPEA (85 mg, 0.66 mmol). The mixture was stirred at 100° C. in a sealed tube for 16 hours. The mixture was cooled and diluted with EtOAc (10 mL), washed with brine (5 mL×3), dried over Na₂SO₄, filtered and concentrated under reduced pressure. The resulting residue was purified by prep-TLC (CH₂Cl₂/MeOH=10:1) and Prep-HPLC (Method A) to give the titled compound (19 mg, 39%). ¹H NMR (500 MHz, DMSO-d6) δ 8.03-7.92 (m, 1H), 7.34-7.26 (m, 1H), 7.26-7.14 (m, 1H), 5.66-5.55 (m, 2H), 5.38 (s, 1H), 4.83-3.91 (m, 1H), 3.83-3.58 (m, 1H), 3.39-3.32 (m, 1H), 3.29-3.24 (m, 3H), 3.17-2.96 (m, 1H), 2.90-2.75 (m, 1H), 2.61-2,51 (m, 4H), 2.35-2.14 (m, 1H), 2.02-1.78 (m, 2H), 1.67-1.34 (m, 3H), 1.31-1.16 (m, 3H), 0.99-0.77 (m, 5H), 0.69-0.50 (m, 5H). MS: M/e 488 (M+1)⁺.

### Compound A59: 2-(7-((2S,5R)-2,5-diethyl-4-(1-(5-methoxypyridin-2-yl)ethyl)piperazin-1-yl)-4methyl-5-oxo-4,5-dihydro-2H-pyrazolo[4,3-b]pyridin-2-yl)acetonitrile

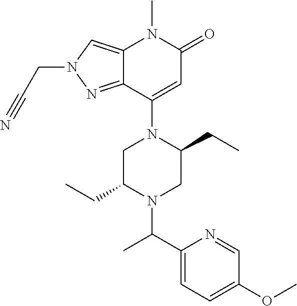 [SMILES: CC[C@H]1CN(C(C)c2ccc(OC)cn2)[C@H](CC)CN1c1cc(=O)n(C)c2cn(CC#N)nc12]

### Step A: 5-methoxypicolinic acid

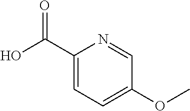

To a stirred solution of methyl 5-methoxypicolinate (500 mg, 3 mmol) in MeOH (8 mL) was added H₂O (2 mL), followed by NaOH (240 mg, 6 mmol). After then, the mixture was stirred for 3 hours. The reaction mixture was concentrated to give the aqueous layer, which was acidified to pH=3˜4 with aq·HCl, then extracted with CH₂Cl₂ (10 mL×6). The combined organic layers were washed with brine, dried over Na₂SO₄, concentrated to give the titled compound (395 mg, 86%). MS: M/e 154 (M+1)⁺.

### Step B: N,5-dimethoxy-N-methylpicolinamide

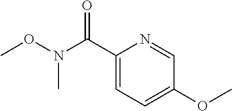

To a stirred solution of 5-methoxypicolinic acid (395 mg, 2,58 mmol), N,O-dimethylhydroxylamine hydrochloride (302 mg, 3.1 mmol), HATU (1.18 g, 3.1 mmol) and DIPEA (665 mg, 5.16 mmol) was stirred overnight. The reaction mixture was washed with H₂O, extracted with CH₂Cl₂ (15 mL×2). The combined organic layers were washed with brine, dried over Na₂SO₄, concentrated to dryness. The resulting residue was purified by flash column chromatography to give the titled compound (crude, 100%). MS: M/e 197 (M+1)⁺.

### Step C: 1-(5-methoxypyridin-2-yl)ethan-1-one

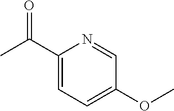

To a stirred solution of N,5-dimethoxy-N-methylpicolinamide (crude, 2,58 mmol) in THF (15 mL) was added dropwise MeMgBr (3.0 M, 1 mL, 3.1 mmol) at 0° C. After then, the mixture was stirred for 30 min. The reaction was quenched with aq·NH₄Cl, extracted with EtOAc (15 mL×3). The combined organic layers were washed with brine, dried over Na₂SO₄, concentrated to dryness. The resulting residue was purified by flash column chromatography to give the titled compound (248 mg, 63.6%). MS: M/e 152 (M+1)⁺.

### Step D: 1-(5-methoxypyridin-2-yl)ethan-1-ol

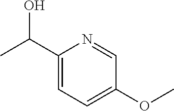

To a stirred solution of 1-(5-methoxypyridin-2-yl)ethan-1-one (248 mg, 1.64 mmol) in MeOH (5 mL) was added NaBH₄ (62.3 mg, 1.64 mmol). After then, the reaction mixture was stirred for 10 min. The reaction mixture was quenched with H₂O, then most MeOH was removed to give the aqueous layer, then extracted with EtOAc (10 mL×3). The combined organic layers were washed with brine, dried over Na₂SO₄, concentrated to give the titled compound (248 mg, 100%). MS: M/e 154 (M+1)⁺.

### Step E: 2-(7-((2S,5R)-2,5-diethyl-4-(1-(5-methoxypyridin-2-yl)ethyl)piperazin-1-vi)-4-methyl-5-oxo-4,5-dihydro-2H-pyrazolo[4,3-b]pyridin-2-yl)acetonitrile

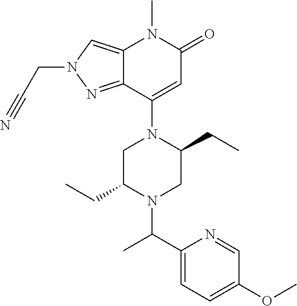 [SMILES: CC[C@H]1CN(C(C)c2ccc(OC)cn2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), 1-(5-methoxypyridin-2-yl)ethan-1-ol (30.6 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred overnight at 100° C. in a sealed tube. The reaction mixture was diluted with EtOAc (10 mL), then washed with brine, dried over Na₂SO₄, concentrated to dryness. The resulting residue was purified by Pre-TLC (CH₂Cl₂/MeOH=10:1) to give the titled compound (15 mg). ¹H NMR (500 MHz, CD₃OD) δ 8.19-8.13 (m, 1H), 7.93 (d, J=4.4 Hz, 1H), 7.54 (dd, J=18.0, 8.8 Hz, 1H), 7.47-7.42 (m, 1H), 5.56 (d, J=2.8 Hz, 1H), 5.47 (d, J=4.8 Hz, 2H), 3.99-3.91 (m, 1H), 3.88 (d, J=4.0 Hz, 3H), 3.59-3.46 (m, 1H), 3.43 (d, J=1,2 Hz, 3H), 3.38-3.34 (m, 1H), 3.23-2.75 (m, 2H), 2,49-2.24 (m, 1H), 2.14-1.77 (m, 2H), 1.76-1.51 (m, 3H), 1.37 (dd, J=11.6, 6.4 Hz, 3H), 1.08-0.92 (m, 3H), 0.77-0.63 (m, 3H) ppm. MS: M/e 464 (M+1)⁺.

### Compound A60: 2-(7-((2S,5R)-2,5-diethyl-4-(1-(5-methoxy-3-methylpyridin-2-yl)ethyl)piperazin-1-yl)-4-methyl-5-oxo-4,5-dihydro-2H-pyrazolo[4,3-b]pyridin-2-y acetonitrile

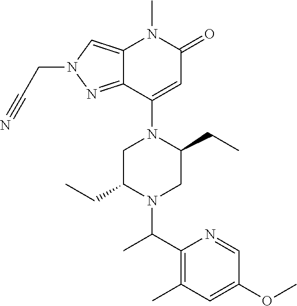 [SMILES: CC[C@H]1CN(C(C)c2ncc(OC)cc2C)[C@H](CC)CN1c1cc(=O)n(C)c2cn(CC#N)nc12]

### Step A: 5-methoxy-3-methylpicolinic acid

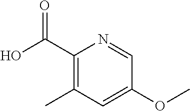

To a stirred solution of methyl 5-methoxy-3-methylpicolinate (500 mg, 2.76 mmol) in MeOH (8 mL) was added H₂O (2 mL), followed by NaOH (221 mg, 5.52 mmol). After then, the mixture was stirred for 3 hours. The reaction mixture was concentrated to give the aqueous layer, which was acidified to pH=3-4 with aq·HCl, then extracted with CH₂Cl₂ (15 mL×6). The combined organic layers were washed with brine, dried over Na₂SO₄, concentrated to give the titled compound (460 mg, 100%). MS: M/e 168 (M+1)⁺.

### Step B: N,5-dimethoxy-N,3-dimethylpicolinamide

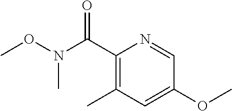

To a stirred solution of 5-methoxy-3-methylpicolinic acid (460 mg, 2.75 mmol), N,O-dimethylhydroxylamine hydrochloride (322 mg, 3.3 mmol), HATU (1,26 g, 3.3 mmol) and DIPEA (710 mg, 5.5 mmol) was stirred overnight. The reaction mixture was washed with H₂O, extracted with CH₂Cl₂ (15 mL×2). The combined organic layers were washed with brine, dried over Na₂SO₄, concentrated to dryness. The resulting residue was purified by flash column chromatography to give the titled compound (520 mg, 90%). MS: M/e 211 (M+1)⁺.

### Step C: 1-(5-methoxy-3-methylpyridin-2-yl)ethan-1-one

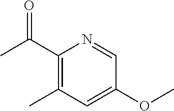

To a stirred solution of N,5-dimethoxy-N,3-dimethylpicolinamide (300 mg, 1.82 mmol) in MeOH (10 mL) was added NaBH₄ (69 mg, 1.82 mmol). After then, the reaction mixture was stirred for 10 min. The reaction mixture was quenched with H₂O, then most MeOH was removed to give the aqueous layer, then extracted with EtOAc (10 mL×2). The combined organic layers were washed with brine, dried over Na₂SO₄, concentrated to give the titled compound (240 mg, 79%). MS: We 166 (M+1)⁺.

### Step D: 1-(5-methoxy-3-methylpyridin-2-yl)ethan-1-ol

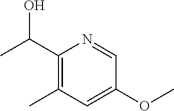

To a stirred solution of 1-(5-methoxy-3-methylpyridin-2-yl)ethan-1-one (248 mg, 1.64 mmol) in MeOH (5 mL) was added NaBH₄ (62.3 mg, 1.64 mmol). After then, the reaction mixture was stirred for 10 min. The reaction mixture was quenched with H₂O, then most MeOH was removed to give the aqueous layer, then extracted with EtOAc (10 mL×3). The combined organic layers were washed with brine, dried over Na₂SO₄, concentrated to give the titled compound (248 mg, 100%). MS: We 168 (M+1)⁺.

### Step E: 2-(7-((2S,5R)-2,5-diethyl-4-(1-(5-methoxy-3-methylpyridin-2-yl)ethyl)piperazin-1-yl)-4-methyl-5-oxo-4,5-dihydro-2H-pyrazolo[4,3-b]pyridin-2-yl)acetonitrile

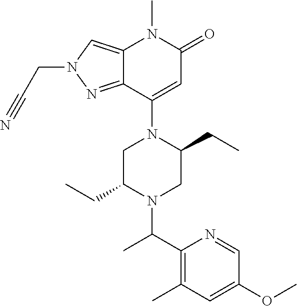 [SMILES: CC[C@H]1CN(C(C)c2ncc(OC)cc2C)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), 1-(5-methoxy-3-methylpyridin-2-yl)ethan-1-ol (33.4 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred overnight at 100° C. in a sealed tube. The reaction mixture was diluted with EtOAc (10 mL), then washed with brine, dried over Na₂SO₄, concentrated to dryness. The resulting residue was purified by Prep-HPLC (Method A) to give the titled compound (8 mg) as a formic acid salt. ¹H NMR (500 MHz, CD₃OD) δ 8.39 (s, 1H), 8.15-8.02 (m, 1H), 7.97-7.91 (m, 1H), 7.29-7.22 (m, 1H), 5.62-5.54 (m, 1H), 5.50-5.44 (m, 2H), 4.22-4.16 (m, 0.5H), 3.91-3.83 (m, 3H), 3.70-3.63 (m, 0.5H), 3.47-3.42 (m, 3H), 3.41-3.34 (m, 2H), 3.17-2.92 (m, 2H), 2.63-2,52 (m, 1H), 2,52-2,42 (m, 3H), 2.00-1.49 (m, 5H), 1.46 (t, J=6.8 Hz, 3H), 1.12-0.83 (m, 3H), 0.76-0.66 (m, 3H) ppm. MS: M/e 478 (M+1)⁺.

### Compound A60: 2-(7-((2S,5R)-2,5-diethyl-4-(1-(5-methoxy-3-methylpyridin-2-yl)ethyl)piperazin-1-yl)-4-methyl-5-oxo-4,5-dihydro-2H-pyrazolo[4,3-b]pyridin-2-yl)acetonitrile

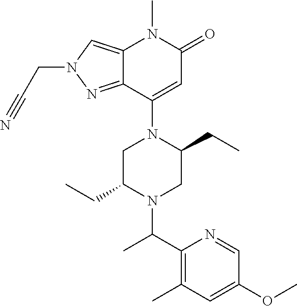 [SMILES: CC[C@H]1CN(C(C)c2ncc(OC)cc2C)[C@H](CC)CN1c1cc(=O)n(C)c2cn(CC#N)nc12]

### Step A: 5-methoxy-3-methylpicolinic acid

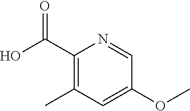

To a stirred solution of methyl 5-methoxy-3-methylpicolinate (500 mg, 2.76 mmol) in MeOH (8 mL) was added H₂O (2 mL), followed by NaOH (221 mg, 5.52 mmol). After then, the mixture was stirred for 3 hours. The reaction mixture was concentrated to give the aqueous layer, which was acidified to pH=3-4 with aq·HCl, then extracted with CH₂Cl₂ (15 mL×6). The combined organic layers were washed with brine, dried over Na₂SO₄, concentrated to give the titled compound (460 mg, 100%). MS: M/e 168 (M+1)⁺.

### Step B: N,5-dimethoxy-N,3-dimethylpicolinamide

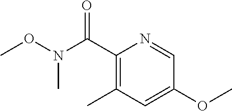

To a stirred solution of 5-methoxy-3-methylpicolinic acid (460 mg, 2.75 mmol), N,O-dimethylhydroxylamine hydrochloride (322 mg, 3.3 mmol), HATU (1,26 g, 3.3 mmol) and DIPEA (710 mg, 5.5 mmol) was stirred overnight. The reaction mixture was washed with H₂O, extracted with CH₂Cl₂ (15 mL×2). The combined organic layers were washed with brine, dried over Na₂SO₄, concentrated to dryness. The resulting residue was purified by flash column chromatography to give the titled compound (520 mg, 90%). MS: M/e 211 (M+1)⁺.

### Step C: 1-(5-methoxy-3-methylpyridin-2-yl)ethan-1-one

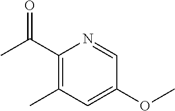

To a stirred solution of N,5-dimethoxy-N,3-dimethylpicolinamide (300 mg, 1.82 mmol) in MeOH (10 mL) was added NaBH₄ (69 mg, 1.82 mmol). After then, the reaction mixture was stirred for 10 min. The reaction mixture was quenched with H₂O, then most MeOH was removed to give the aqueous layer, then extracted with EtOAc (10 mL×2). The combined organic layers were washed with brine, dried over Na₂SO₄, concentrated to give the titled compound (240 mg, 79%). MS: We 166 (M+1)⁺.

### Step D: 1-(5-methoxy-3-methylpyridin-2-yl)ethan-1-ol

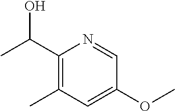

To a stirred solution of 1-(5-methoxy-3-methylpyridin-2-yl)ethan-1-one (248 mg, 1.64 mmol) in MeOH (5 mL) was added NaBH₄ (62.3 mg, 1.64 mmol). After then, the reaction mixture was stirred for 10 min. The reaction mixture was quenched with H₂O, then most MeOH was removed to give the aqueous layer, then extracted with EtOAc (10 mL×3). The combined organic layers were washed with brine, dried over Na₂SO₄, concentrated to give the titled compound (248 mg, 100%). MS: We 168 (M+1)⁺.

### Step E: 2-(7-((2S,5R)-2,5-diethyl-4-(1-(5-methoxy-3-methylpyridin-2-yl)ethyl)piperazin-1-yl-4-methyl-5-oxo-4,5-dihydro-2H-pyrazolo[4,3-b]pyridin-2-yl)acetonitrile

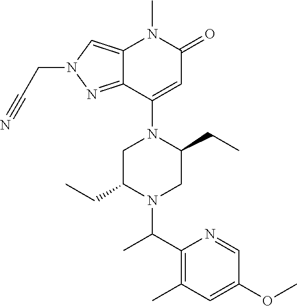 [SMILES: CC[C@H]1CN(C(C)c2ncc(OC)cc2C)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), 1-(5-methoxy-3-methylpyridin-2-yl)ethan-1-ol (33.4 mg, 0.2 mmol), (cyanomethyl)trimethylphosphonium iodide (72.9 mg, 0.3 mmol) and DIPEA (64.5 mg, 0.5 mmol) in CH₃CN (4 mL) was stirred overnight at 100° C. in a sealed tube. The reaction mixture was diluted with EtOAc (10 mL), then washed with brine, dried over Na₂SO₄, concentrated to dryness. The resulting residue was purified by Prep-HPLC (Method A) to give the titled compound (8 mg) as a formic acid salt. ¹H NMR (500 MHz, CD₃OD) δ 8.39 (s, 1H), 8.15-8.02 (m, 1H), 7.97-7.91 (m, 1H), 7.29-7.22 (m, 1H), 5.62-5.54 (m, 1H), 5.50-5.44 (m, 2H), 4.22-4.16 (m, 0.5H), 3.91-3.83 (m, 3H), 3.70-3.63 (m, 0.5H), 3.47-3.42 (m, 3H), 3.41-3.34 (m, 2H), 3.17-2.92 (m, 2H), 2.63-2,52 (m, 1H), 2,52-2,42 (m, 3H), 2.00-1.49 (m, 5H), 1.46 (t, J=6.8 Hz, 3H), 1.12-0.83 (m, 3H), 0.76-0.66 (m, 3H) ppm. MS: M/e 478 (M+1)⁺.

### Compound A61: 2-(7-((2S,5R)-2,5-diethyl-4-(1-(5-(methoxymethyl)pyridin-2-yl)ethyl)piperazin-1-yl)-4-methyl-5-oxo-4,5-dihydro-2H-pyrazolo[4,3-b]pyridin-2-yl)acetonitrile

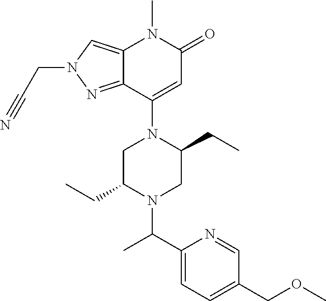 [SMILES: CC[C@H]1CN(C(C)c2ccc(COC)cn2)[C@H](CC)CN1c1cc(=O)n(C)c2cn(CC#N)nc12]

### Step A: methyl 5-4methoxymethyl)picolinate

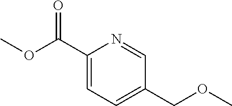

To a stirred solution of methyl 5-(hydroxymethyl)picolinate (250 mg, 1.49 mmol) in toluene (5 mL) was added SOCl₂ (266 mg, 2.2 mmol). After then, the mixture was stirred at 80° C. for 4 hours. The reaction mixture was concentrated to give the intermediate, which was dissolved in MeOH (5 mL), followed by MeONa (2.0 M, 4 mL, 8 mmol). After the addition, the reaction was stirred overnight. The reaction mixture was directly used to the next step. MS: M/e 182 (M+1)⁺.

### Step B: 5-4methoxymethyl)picolinic acid

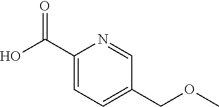

To the previous mixture was added H₂O (2 mL), and then stirred for 2 hours. The mixture was acidified to pH=6-7 with aq·HCl and concentrated to give the crude product, which was directly used to the next step. MS: M/e 168 (M+1)⁺.

### Step C: N-methoxy-5-(methoxymethyl)-N-methylpicolinamide

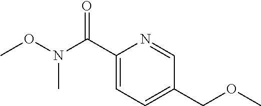

To a stirred mixture of crude 5-(methoxymethyl)picolinic acid (1.49 mmol) in CH₂Cl₂ (10 mL) was added N,O-dimethylhydroxylamine hydrochloride (175 mg, 1.8 mmol), HATU (687.6 mg, 1.8 mmol) and DIPEA (387 mg, 3 mmol). After the addition, the reaction was stirred overnight. The reaction mixture was washed with H₂O, extracted with CH₂Cl₂ (10 mL×3). The combined organic layers were washed with brine, dried over Na₂SO₄, concentrated to dryness. The resulting residue was purified by flash column chromatography to give the titled compound (180 mg, 57.5%). MS: M/e 211 (M+1)⁺.

### Step D: 1-(5-(methoxymethyl)pyridin-2-yl)ethan-1-one

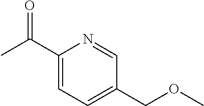

To a stirred solution of N-methoxy-5-(methoxymethyl)-N-methylpicolinamide (180 mg, 0.86 mmol) in THF (5 mL) was added dropwise MeMgBr (3.0 M, 0.34 mL, 1.03 mmol) at 0° C. After the addition, the reaction mixture was stirred for an hour. The reaction was quenched with aq·NH₄Cl, extracted with EtOAc (15 mL×3). The combined organic layers were washed with brine, dried over Na₂SO₄, concentrated to dryness. The resulting residue was purified by flash column chromatography to give the titled compound (80 mg, 56.3%). MS: M/e 166 (M+1)⁺.

### Step E: 1-(5-(methoxymethyl)pyridin-2-yl)ethan-1-ol

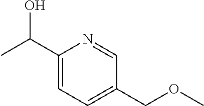

To a stirred solution of 1-(5-(methoxymethyl)pyridin-2-yl)ethan-1-one (80 mg, 0.48 mmol) in MeOH (5 mL) was added NaBH₄ (18 mg, 0.48 mmol). After stirred for 10 min, the reaction mixture was concentrated, then treated with H₂O, extracted with EtOAc (10 mL×2). The combined organic layers were washed with brine, dried over Na₂SO₄, concentrated to give the titled compound (78 mg, 97.3%).

### Step F: 2-(7-((2S,5R)-2,5-diethyl-4-(1-(5-(methoxymethyl)pyridin-2-yl)ethyl)piperazin-1-yl)-4-methyl-5-oxo-4,5-dihydro-2H-pyrazolo[4,3-b]pyridin-2-yl)acetonitrile

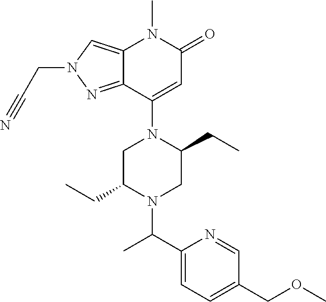 [SMILES: CC[C@H]1CN(C(C)c2ccc(COC)cn2)[C@H](CC)CN1c1cc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), 1-(5-(methoxymethyl)pyridin-2-yl)ethan-1-ol (33.4 mg, 0.2 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄, concentrated to dryness. The resulting residue was purified by Pre-TLC (CH₂Cl₂/MeOH=10:1) to give the titled compound(16 mg). ¹H NMR (500 MHz, CD₃OD) δ 8.45 (s, 1H), 7.94-7.91 (m, 1H), 7.86-7.81 (m, 1H), 7.66-7.59 (m, 1H), 5.56 (s, 1H), 5.49-5.44 (m, 2H), 4.52-4.49 (m, 2H), 4.03-3.94 (m, 0.5H), 3.60-3.51 (m, 0.5H), 3.44-3.43 (m, 3H), 3.42-3.41 (m, 3H), 3.38-3.32 (m, 1H), 3.26-3.16 (m, 1H), 3.06-2.75 (m, 2H), 2,47-2.23 (m, 1H), 2.16-1.79 (m, 2H), 1.79-1.51 (m, 3H), 1.43-1.35 (m, 3H), 1.07-0.92 (m, 3H), 0.76-0.63 (m, 3H) ppm. MS: M/e 478 (M+1)⁺.

### Compound A62: 2-(7-((2S,5R)-4-(1-(5-cyclopropyl-6-fluoropyridin-2-yl)ethyl)-2,5-diethylpiperazin-1-yl)-4-methyl-5-oxo-4,5-dihydro-2H-pyrazolo[4,3-b]pyridin-2-yl)acetonitrile

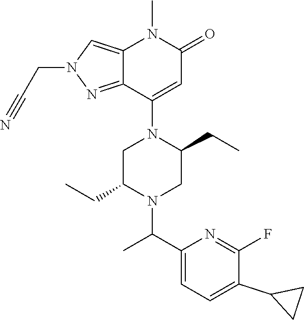 [SMILES: CC[C@H]1CN(C(C)c2ccc(C3CC3)c(F)n2)[C@H](CC)CN1c1cc(=O)n(C)c2cn(CC#N)nc12]

### Step A: 6-chloro-3-cyclopropyl-2-fluoropyridine

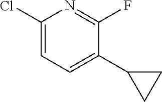 [SMILES: Fc1nc(Cl)ccc1C1CC1]

To a solution of 3-bromo-6-chloro-2-fluoropyridine (0.63 g, 3 mmol) and cyclopropylboronic acid (284 mg, 3.3 mmol) in dioxane/H₂O (10 mL/1 mL) were added Pd(dppf)Cl₂ (220 mg, 0.3 mmol) and K₂CO₃ (828 mg, 6 mmol). The reaction mixture was stirred at 100° C. under N₂ overnight. The mixture was cooled to room temperature, diluted with water (40 mL), extracted with EA (60 mL×2), washed with brine, dried over Na₂SO₄, filtered, and concentrated to dryness. The resulting residue was purified by flash column chromatography to give the titled compound (310 mg, 60%). MS: M/e 172 (M+1)⁺.

### Step B: 1-(5-cyclopropyl-6-fluoropyridin-2-yl)ethan-1-one

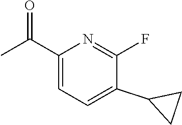

To a solution of 6-chloro-3-cyclopropyl-2-fluoropyridine (310 mg, 1.8 mmol) in toluene (6 mL) was added tributyl(1-ethoxyvinyl)stannane (850 mg, 2.7 mmol) and Pd(PPh₂)₂Cl₂ (63 mg, 0.09 mmol). The reaction mixture was protected by N₂ atmosphere and stirred at 100° C. overnight. The mixture was cooled down to RT, diluted with EA (10 mL), a solution of 4M HCl in dioxane (2 mL) was added. The resulting mixture was stirred at RT for 1 hour. The reaction was quenched with H₂O and saturated NaHCO₃ solution, extracted with EA (40 mL×2), washed with brine, dried over Na₂SO₄, filtered, and concentrated to dryness. The resulting residue was purified by flash column chromatography to give the titled compound (160 mg, 50%). MS: M/e 180 (M+1)⁺.

### Step C: 1-(5-cyclopropyl-6-fluoropyridin-2-yl)ethan-1-ol

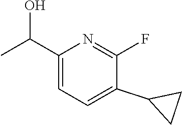

To a solution of 1-(5-cyclopropyl-6-fluoropyridin-2-yl)ethan-1-one (160 mg, 0.9 mmol) in MeOH (5 mL) was added NaBH₄ (34 mg, 0.89 mmol) slowly at 0° C. The resulting mixture was stirred at room temperature for 1 hour. The reaction mixture was quenched with H₂O (10 mL) and extracted with EA (60 mL), washed with brine, dried over Na₂SO₄, filtered, and concentrated to dryness. The resulting residue was purified by flash column chromatography to give the desired product (130 mg, 81%). ¹H NMR (400 MHz, DMSO-d₆) δ 7.58-7.50 (m, 1H), 7.31 (d, J=8.0 Hz, 1H), 5.39 (d, J=4.4 Hz, 1H), 4.65-4.54 (m, 1H), 2.00-1.90 (m, 1H), 1.31 (d, J=6.4 Hz, 3H), 1.02-0.90 (m, 2H), 0.78-0.70 (m, 2H) ppm.

### Step D: 2-(7-((2S,5R)-4-(1-5-cyclopropyl-6-fluoropyridin-2-yl)ethyl)-2,5-diethylpiperazin-1-yl)-4-methyl-5-oxo-4,5-dihydro-2H-pyrazolo[4,3-b]pyridin-2-yl)acetonitrile

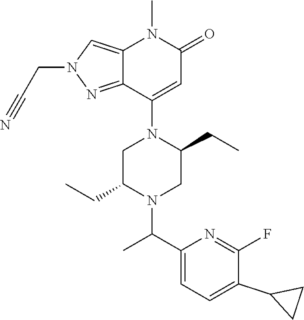 [SMILES: CC[C@H]1CN(C(C)c2ccc(C3CC3)c(F)n2)[C@H](CC)CN1c1cc(=O)n(C)c2cn(CC#N)nc12]

To a solution of 1-(5-cyclopropyl-6-fluoropyridin-2-yl)ethan-1-ol (36 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.6 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-HPLC (Method A) to give the titled compound (30 mg, 61%). ¹H NMR (400 MHz, CD₃OD) δ 7.92 (s, 1H), 7.56-7.45 (m, 1H), 7.42-7.28 (m, 1H), 5.55 (s, 1H), 5.47 (s, 2H), 3.88-3.78 (m, 0.5H), 3.71-3.62 (m, 0.5H), 3.68-3.58 (m, 0.5 H), 3.56-3.47 (m, 0.5H), 3.43 (s, 3H), 3.38-3.33 (m, 0.5H), 3.35-3.31 (m, 1.5H), 3.16-3.06 (m, 0.5H), 2.98-2.93 (m, 1H), 2.82-2.71 (m, 0.5H), 2,51-2.30 (m, 1H), 2.15-1.45 (m, 5H), 1.41-1,25 (m, 3H), 1.07-0.88 (m, 5H), 0.81-0.62 (m, 5H) ppm. MS: M/e 492 (M+1)⁺.

### Compound A63: 2-(7-((2S,5R)-4-(1-(5-cyclopropyl-4-fluoropyridin-2-yl)ethyl)-2,5-diethylpiperazin-1-yl)-4-methyl-5-oxo-4,5-dihydro-2H-pyrazolo[4,3-b]pyridin-2-yl)acetonitrile

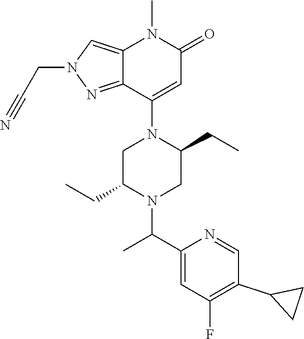 [SMILES: CC[C@H]1CN(C(C)c2cc(F)c(C3CC3)cn2)[C@H](CC)CN1c1cc(=O)n(C)c2cn(CC#N)nc12]

### Step A: 4-fluoro-5-iodopyridin-2-amine

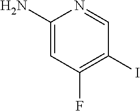

At 0° C., to a solution of 4-fluoropyridin-2-amine (2.24 g, 20 mmol) and TFA (0.91 g, 8 mmol) in CH₃CN (40 mL) was added a suspension of NIS (5.15 g, 23 mmol) in CH₃CN (10 mL). The reaction mixture was stirred at room temperature overnight. The mixture diluted with water (20 mL) and saturated NaHCO₃ solution (20 mL), extracted with EA (80 mL), washed with saturated NaHSO₃ solution, brine, dried over Na₂SO₄, filtered, and concentrated to dryness. The resulting residue was purified by flash column chromatography to give the titled compound (3.3 g, 69%). ¹H NMR (500 MHz, CDCl₃) δ 8.23 (d, J=9.6 Hz, 1H), 6.24 (d, J=9.6 Hz, 1H), 4.62 (br s, 2H) ppm.

### Step B: 5-cyclopropyl-4-fluoropyridin-2-amine

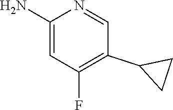 [SMILES: Nc1cc(F)c(C2CC2)cn1]

To a solution of 4-fluoro-5-iodopyridin-2-amine (3.2 g, 13.44 mmol), cyclopropylboronic acid (1.15 g, 20.2 mmol) and K₃PO₄ (6 g, 26.9 mmol) in toluene/H₂O (20 mL/ 2 mL) was added sphos (550 mg, 1.34 mmol) and Pd(OAc)₂ (330 mg, 1.34 mmol). The reaction was stirred at 100° C. overnight under N₂ protect. The mixture was diluted with H₂O (60 mL), extracted with EA (80 mL×2), washed with brine, dried over Na₂SO₄, filtered, and concentrated to dryness. The resulting residue was purified by flash column chromatography to give the titled compound (1.5 g, 70%). MS: M/e 153 (M+1)⁺.

### Step C: 2-bromo-5-cyclopropyl-4-fluoropyridine

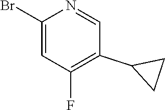 [SMILES: Fc1cc(Br)ncc1C1CC1]

To a solution of 5-cyclopropyl-4-fluoropyridin-2-amine (760 mg, 5 mmol) in CH₂Br₂ (20 mL) at 0° C. was added CuBr₂ (2.23 g, 10 mmol) and tert-Butyl nitrite (1.03 g, 10 mmol) drop wise. The reaction mixture was slowly warmed to room temperature overnight under N₂ protect. The mixture was quenched with H₂O/EA, filtered through a pad of Celite, washed with EA (80 mL). The organic layer was washed with brine, dried over Na₂SO₄, filtered, and concentrated to dryness. The resulting residue was purified by flash column chromatography to give the titled compound (560 mg, 52%). MS: M/e 216 (M+1)⁺, 218 (M+3)⁺.

### Step D: 1-(5-cyclopropyl-4-fluoropyridin-2-yl)ethan-1-one

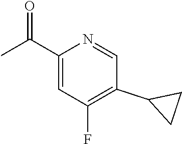

To a solution of 2-bromo-5-cyclopropyl-4-fluoropyridine (560 mg, 2.6 mmol) in toluene (10 mL) was added tributyl(1-ethoxyvinyl)stannane (1,23 g, 3.9 mmol) and Pd(PPh₂)₂Cl₂ (91 mg, 0.13 mmol). The reaction mixture was protected by N₂ atmosphere and stirred at 100° C. overnight. The mixture was cooled down to RT, a solution of 4M HCl in dioxane (2 mL) was added. The resulting mixture was stirred at RT for 2 hours. The reaction was quenched with H₂O and saturated NaHCO₃ solution, extracted with EA (60 mL×2), washed with brine, dried over Na₂SO₄, filtered, and concentrated to dryness. The resulting residue was purified by flash column chromatography to give the titled compound (235 mg, 50%). MS: M/e 180 (M+1)⁺.

### Step E: 1-45-cyclopropyl-4-fluoropyridin-2-yl)ethan-1-ol

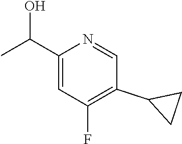

To a solution of 1-(5-cyclopropyl-4-fluoropyridin-2-yl)ethan-1-one (235 mg, 1.31 mmol) in MeOH (5 mL) was added NaBH₄ (50 mg, 1.31 mmol) slowly at 0° C. The resulting mixture was stirred at room temperature for 1 hour. The reaction mixture was quenched with H₂O (10 mL) and extracted with EA (80 mL), washed with brine, dried over Na₂SO₄, filtered, and concentrated to dryness. The resulting residue was purified by flash column chromatography to give the desired product (200 mg, 84%). ¹H NMR (400 MHz, DMSO-d₆) δ 8.19 (d, J=10.8 Hz, 1H), 7.22 (d, J=11.6 Hz, 1H), 5.42 (d, J=4.8 Hz, 1H), 4.75-4.62 (m, 1H), 2.02-1.88 (m, 1H), 1.32 (d, J=6.8 Hz, 3H), 1.02-0.92 (m, 2H), 0.86-0.76 (m, 2H) ppm.

### Step F: 2-(7-((2S,5R)-4-(1-(5-cyclopropyl-4-fluoropyridin-2-yl)ethyl)-2,5-diethylpiperazin-1-yl-4-methyl-5-oxo-4,5-dihydro-2H-pyrazolo[4,3-b]pyridin-2-yl)acetonitrile

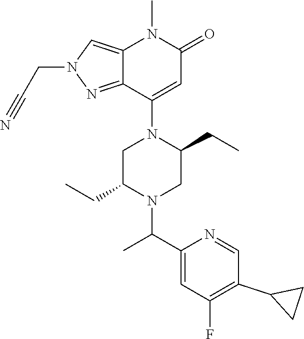 [SMILES: CC[C@H]1CN(C(C)c2cc(F)c(C3CC3)cn2)[C@H](CC)CN1c1cc(=O)n(C)c2cn(CC#N)nc12]

To a solution of 1-(5-cyclopropyl-4-fluoropyridin-2-yl)ethan-1-ol (36 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.6 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-HPLC (Method A) to give the titled compound (28 mg, 57%). ¹H NMR (400 MHz, CD₃OD) δ 8.19-8.07 (m, 1H), 7.91 (s, 1H), 7.39-7.22 (m, 1H), 5.55 (s, 1H), 5.45 (s, 2H), 3.97-3.84 (m, 0.5H), 3.77-3.66 (m, 0.5H), 3.60-3.44 (m, 0.5 H), 3.42 (s, 3H), 3.37-3.33 (m, 0.5H), 3.35-3.31 (m, 2H), 3.16-3.06 (m, 0.5H), 3.02-2.86 (m, 1H), 2.79-2.71 (m, 0.5H), 2,43-2.20 (m, 1H), 2.17-1.45 (m, 5H), 1.37-1,28 (m, 3H), 1.10-0.88 (m, 5H), 0.86-0.62 (m, 5H) ppm. MS: M/e 492 (M+1)⁺.

### Compound A64: 2-(7-((2S,5R)-2,5-diethyl-4-(1-(7-fluoro-2,2-dimethyl-2,3-dihydrobenzo[b][1,4]dioxin-6-yl)ethyl)piperazin-1-yl)-4-methyl-5-oxo-4,5-dihydro-2H-pyrazolo[4,3-b]pyridin-2-yl)acetonitrile

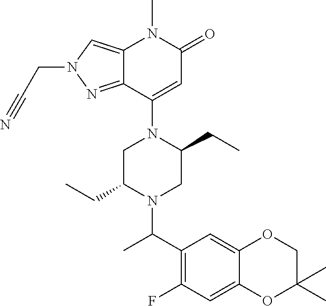 [SMILES: CC[C@H]1CN(C(C)c2cc3c(cc2F)OC(C)(C)CO3)[C@H](CC)CN1c1cc(=O)n(C)c2cn(CC#N)nc12]

### Step A: ethyl 2-(5-bromo-2,4-difluorophenoxy)-2-methylpropanoate

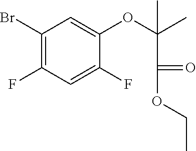 [SMILES: CCOC(=O)C(C)(C)Oc1cc(Br)c(F)cc1F]

A solution of 5-bromo-2,4-difluorophenol (600 mg, 2.87 mmol), ethyl 2-bromo-2-methylpropanoate (672 mg, 3.45 mmol) and K₂CO₃ (792 mg, 5.74 mmol) in DMF (6 ml) was stirred at 70° C. overnight. The solution was diluted with EA (20 ml), washed with brine (10 ml×2), dried over Na₂SO₄ and concentrated to dryness. The resulting residue was purified by flash column chromatography with 0-10% EA in PE to give the titled compound (660 mg, 71%). ¹H NMR (400 MHz, CDCl₃) δ 7.30 (t, J=7.7 Hz, 1H), 6.96-6.89 (m, 1H), 4.26 (q, J=7.2 Hz, 2H), 1.56 (s, 6H), 1.31 (t, J=7.1 Hz, 3H) ppm.

### Step B: 2-(5-bromo-2,4-difluorophenoxy)-2-methylpropan-1-ol

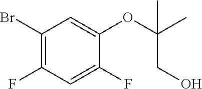

A solution of ethyl 2-(5-bromo-2,4-difluorophenoxy)-2-methylpropanoate (360 mg, 1.11 mmol) and NaBH₄ (126.6 mg, 3.33 mmol) in MeOH (3 ml) was stirred at rt overnight. The solution was diluted with EA (15 ml), washed with brine (10 ml×2), dried over Na₂SO₄ and concentrated to dryness to give the titled compound (300 mg, 96%). ¹H NMR (500 MHz, CDCl₃) δ 7.29 (dd, J=8.3, 7.1 Hz, 1H), 6.96 (dd, J=10.0, 8.1 Hz, 1H), 3.60 (s, 2H), 1,28 (d, J=0.5 Hz, 6H) ppm.

### Step C: 2,4-difluoro-5-((1-hydroxy-2-methylpropan-2-yl)oxy)benzonitrile

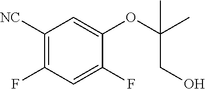 [SMILES: CC(C)(CO)Oc1cc(C#N)c(F)cc1F]

A solution of 2-(5-bromo-2,4-difluorophenoxy)-2-methylpropan-1-ol (300 mg, 1.06 mmol), Zn(CN)₂ (249 mg, 2.13 mmol), tBuXPhosPdG3 (42.2 mg, 0.053 mmol) and tBuXPhos (45.2 mg, 0.11 mmol) in THF (6 ml) was stirred at 70° C. overnight. The solution was diluted with EA (15 ml), washed with brine (10 ml×2), dried over Na₂SO₄ and concentrated to dryness. The resulting residue was purified by flash column chromatography with 0-25% EA in PE to give the titled compound (200 mg, 83%). ¹H NMR (500 MHz, CDCl₃) δ 7.38 (dd, J=8.4, 6.4 Hz, 1H), 7.02 (dd, J=9.6, 8.6 Hz, 1H), 3.63 (s, 2H), 1.30 (s, 6H) ppm. MS: M/e 228 (M+1)⁺.

### Step D: 7-fluoro-2,2-dimethyl-2.3-dihydrobenzo[b][1,4]dioxine-6-carbonitrile

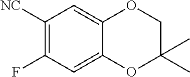

A solution of 2,4-difluoro-5-((1-hydroxy-2-methylpropan-2-yl)oxy)benzonitrile (200 mg, 0.88 mmol) and NaH (106 mg, 2.65 mmol) in THF (4 ml) was stirred at rt for 1 h. The reaction was quenched with brine (10 ml) and then extracted with EA (10 ml×2). The organic layer was dried over Na₂SO₄ and concentrated to dryness. The resulting residue was purified by flash column chromatography with 0-30% EA in PE to give the titled compound (140 mg, 77%). MS: M/e 208 (M+1)⁺.

### Step E: 1-(7-fluoro-2.2-dimethyl-23-dihydrobenzo[b][1,4]dioxin-6-yl)ethan-1-one

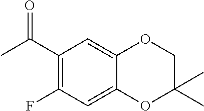 [SMILES: CC(=O)c1cc2c(cc1F)OC(C)(C)CO2]

A solution of 7-fluoro-2,2-dimethyl-2,3-dihydrobenzo[b][1,4]dioxine-6-carbonitrile (140 mg, 0.68 mmol), MeMgBr (3M, 0.68 ml, 2.04 mmol) and CuBr (9.7 mg, 0.068 mmol) in THF (4 ml) was stirred at 60° C. under N₂ overnight. The reaction was quenched with brine (10 ml) and then extracted with EA (10 ml×2). The organic layer was dried over Na₂SO₄ and concentrated to dryness. The resulting residue was purified by flash column chromatography with 0-20% EA in PE to give the titled compound (120 mg, 79%). MS: M/e 225 (M+1)⁺.

### Step F: 147-fluoro-2.2-dimethyl-23-dihydrobenzo[b][1,4]dioxin-6-yl)ethan-1-ol

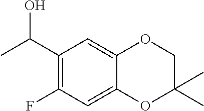 [SMILES: CC(O)c1cc2c(cc1F)OC(C)(C)CO2]

A solution of 1-(7-fluoro-2,2-dimethyl-2,3-dihydrobenzo[b][1,4]dioxin-6-yl)ethan-1-one (120 mg, 0.54 mmol) and NaBH₄ (20 mg, 0.53 mmol) in MeOH (2 ml) was stirred at rt for 15 min. The reaction was quenched with brine (10 ml) and then extracted with EA (10 ml×2). The organic layer was dried over Na₂SO₄ and concentrated to dryness to give the titled compound (120 mg, 99%), which was used directly for the next step without further purification. MS: M/e 227 (M+1)⁺.

### Step G: 2-(7-((2S,5R)-2,5-diethyl-4-(1-(7-fluoro-2.2-dimethyl-23-dihydrobenzo[b][1,4]dioxin-6-yl)ethylpiperazin-1-yl-4-methyl-5-oxo-4,5-dihydro-2H-pyrazolo[4,3-b]pyridin-2-yl)acetonitrile

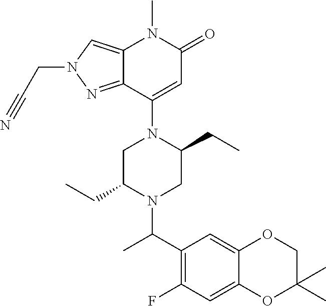 [SMILES: CC[C@H]1CN(C(C)c2cc3c(cc2F)OC(C)(C)CO3)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (58 mg, 0.18 mmol), 1-(7-fluoro-2,2-dimethyl-2,3-dihydrobenzo[b][1,4]dioxin-6-yl)ethan-1-ol (60 mg, 0.27 mmol), (cyanomethyl)trimethylphosphonium iodide (129 mg, 0.53 mmol) and DIPEA (228 mg, 1.77 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography with 0-10% MeOH in DCM and further purified by Prep-HPLC (Method A) to give the titled compound (19 mg). ¹H NMR (500 MHz, DMSO-d6) δ 8.43 (s, 0.18H), 7.98 (d, J=3.1 Hz, 1H), 6.91 (dd, J=38.1, 7.0 Hz, 1H), 6.74 (dd, J=15.2, 10.8 Hz, 1H), 5.61 (s, 2H), 5.39 (s, 1H), 4.00-3.91 (m, 2H), 3.88 (d, J=11.2 Hz, 0.5H), 3.83-3.78 (m, 0.5H), 3.33 (s, 3H), 3.27 (d, J=1,2 Hz, 3H), 3.11 (d, J=12.3 Hz, 0.5H), 2.99 (d, J=8.7 Hz, 0.5H), 2.85-2.71 (m, 1H), 2.36 (s, 0.5H), 2.28 (d, J=12.1 Hz, 0.5H), 2.03-1.84 (m, 1H), 1.64-1.53 (m, 1H), 1.48-1.32 (m, 2H), 1.31-1.19 (m, 9H), 0.92 (dt, J=50, 10 Hz, 3H), 0.65 (dt, J=50, 10 Hz, 3H) ppm. MS: M/e 537 (M+1)⁺.

### Compound A65: 2-(7-((2S,5R)-4-(1-(2,3-dihydro-1H-inden-5-yl)ethyl)-2,5-diethylpiperazin-1-yl)-4-methyl-5-oxo-4,5-dihydro-2H-pyrazolo[4,3-b]pyridin-2-yl)acetonitrile

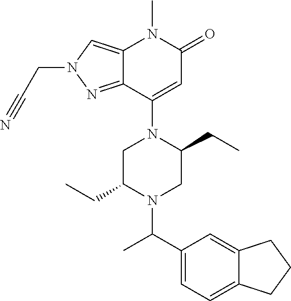 [SMILES: CC[C@H]1CN(C(C)c2ccc3c(c2)CCC3)[C@H](CC)CN1c1cc(=O)n(C)c2cn(CC#N)nc12]

### Step A: 1-(2,3-dihydro-1H-inden-5-yl)ethan-1-ol

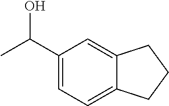

To a solution of 1-(2,3-dihydro-1H-inden-5-yl)ethan-1-one (320 mg, 2 mmol) in MeOH (10 mL) was added NaBH₄ (76 mg, 2 mmol) slowly at 0° C. The resulting mixture was stirred at room temperature for 1 hour. The reaction mixture was quenched with H₂O (20 mL) and extracted with EA (80 mL), washed with brine, dried over Na₂SO₄, filtered, and concentrated to dryness to give the desired product (280 mg, 86%).

### Step B: 2-(7-((2S,5R)-4-(1-(2.3-dihydro-1H-inden-5-yl)ethyl)-2,5-diethylpiperazin-1-yl-4-methyl-5-oxo-4,5-dihydro-2H-pyrazolo[4,3-b]pyridin-2-yl)acetonitrile

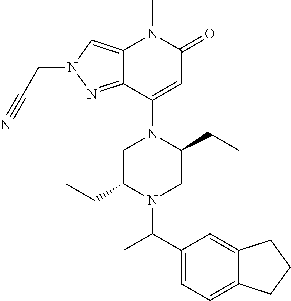 [SMILES: CC[C@H]1CN(C(C)c2ccc3c(c2)CCC3)[C@H](CC)CN1c1cc(=O)n(C)c2cn(CC#N)nc12]

To a solution of 1-(2,3-dihydro-1H-inden-5-yl)ethan-1-ol (34 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (75 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-TLC (DCM/MeOH=20/1) and then Prep-HPLC (Method A) to give the titled compound (7 mg, 14%). ¹H NMR (400 MHz, CD₃OD) δ 7.92 (s, 1H), 7.28-7.04 (m, 3H), 5.54 (s, 1H), 5.46 (s, 2H), 3.75-3.45 (m, 2H), 3.43 (s, 3H), 3.35-3.31 (m, 2H), 3.18-3.09 (m, 1H), 3.04-2.94 (m, 0.5H), 2.95-2.80 (m, 4H), 2.70-2.35 (m, 1.5H), 2.20-1.90 (m, 3H), 1.85-1.43 (m, 3H), 1.36-1,25 (m, 3H), 1.07-0.90 (m, 3H), 0.73-0.58 (m, 3H) ppm. MS: M/e 473 (M+1)”.

### Compound A66: 2-(7-((2S,5R)-4-(1-(1,1-dimethyl-2,3-dihydro-1H-inden-5-yl)ethyl)-2,5-diethylpiperazin-1-yl)-4-methyl-5-oxo-4,5-dihydro-2H-pyrazolo[4,3-b]pyridin-2-yl)acetonitrile

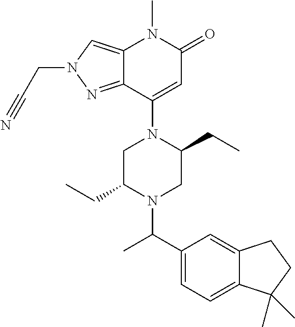 [SMILES: CC[C@H]1CN(C(C)c2ccc3c(c2)CCC3(C)C)[C@H](CC)CN1c1cc(=O)n(C)c2cn(CC#N)nc12]

### Step A: 5-bromo-1.1-dimethyl-2,3-dihydro-1H-indene

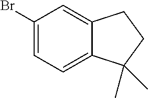

To a stirred-solution of titanium chloride (1 M in DCM, 10 ml, 10 mmol) in anhydrous dichloromethane (30 ml) was added 1.01 M dimethyl zinc toluene solution (10 ml, 10 mmol) at −45° C. under nitrogen and the mixture was stirred for 10 minutes at −45° C. To the mixture was added a solution of 5-bromo-2,3-dihydro-1H-inden-1-one (1.05 g, 5 mmol) in anhydrous dichloromethane (15 ml) dropwise at −45° C. and the mixture was warmed to ambient temperature. After 3 hours at ambient temperature, the mixture was poured into ice-water and the aqueous solution was extracted with ethyl acetate (20 ml×3). The combined solution was washed with brine; dried over sodium sulfate. After filtration to separate solvent and sodium sulfate, the solvent was removed under reduced pressure to give a residue. The resulting residue was purified by flash column chromatography (EtOAc: PE=0-50% in 30 minutes) to give the titled compound (800 mg, 71%). ¹H NMR (400 MHz, CDCl₃) δ 7.27 (dd, J=16.7, 8.6 Hz, 2H), 6.98 (d, J=8.0 Hz, 1H), 2.86 (t, J=7.2 Hz, 2H), 1.91 (t, J=7.2 Hz, 2H), 1,23 (s, 6H) ppm. MS: M/e 225 (M+1)⁺.

### Step B: 1-(1.1-dimethyl-23-dihydro-1H-inden-5-yl)ethan-1-one

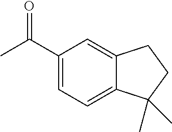

A mixture of 0.5-bromo-1,1-dimethyl-2,3-dihydro-1H-indene (784 mg, 3.5 mmol), tributyl(1-ethoxyvinyl)stannane (1.9 g, 5.25 mmol) and Pd(PPh₃)₂Cl₂ (245 mg, 0.35 mmol) in toluene (5 mL) was stirred at 100° C. under N₂ for 16 hours. To the resulting solution was added HCl (0.5 mL, con.) in drops and the mixture was stirred at room temperature for 1 hour. The mixture was treated with saturated NaHCO₃ aq. (20 ml), extracted with EtOAc(10 ml*3), washed with brine (20 mL), dried and concentrated to dryness. The resulting residue was purified by flash column chromatography (EtOAc: PE=0-50% in 30 minutes) to give the titled compound (500 mg, 76%). MS: M/e 189 (M+1)⁺.

### Step C: 1-(1.1-dimethyl-2.3-dihydro-1H-inden-5-yl)ethan-1-ol

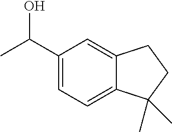

To a solution of 1-(1,1-dimethyl-2,3-dihydro-1H-inden-5-yl)ethan-1-one (490 mg, 2.6 mmol) in MeOH (5 mL) was added NaBH₄ (79 mg, 2.1 mmol) at room temperature and the mixture was stirred at room temperature for 30 minutes. The resulting mixture was treated with water(30 ml), extracted with DCM (10 mL×2). The combined organic layers were dried over Na₂SO₄ and concentrated to dryness. The resulting residue (450 mg, crude) was used in the next step. MS: M/e 191 (M+1)⁺.

### Step D: 2-(7-((2S,5R)-4-(1-(1.1-dimethyl-2.3-dihydro-1H-inden-5-yl)ethyl)-2,5-diethylpiperazin-1-yl-4-methyl-5-oxo-4,5-dihydro-2H-pyrazolo[4,3-b]pyridin-2-yl)acetonitrile

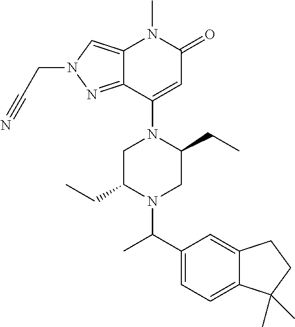 [SMILES: CC[C@H]1CN(C(C)c2ccc3c(c2)CCC3(C)C)[C@H](CC)CN1c1cc(=O)n(C)c2cn(CC#N)nc12]

A mixture of 1-(1,1-dimethyl-2,3-dihydro-1H-inden-5-yl)ethan-1-ol (38 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (72 mg, 0.3 mmol) and DIPEA (65 mg, 0.5 mmol) in MeCN (2 mL) was stirred at 100° C. overnight. The reaction mixture was diluted with EtOAc (20 mL), washed with water (10 mL×2), dried over Na₂SO₄ and concentrated to dryness. The resulting residue was purified by Prep-HPLC (Method A) to give the titled compound (0.75 mg, 1.5%). ¹H NMR (500 MHz, CD₃OD) δ 7.91 (d, J=1.9 Hz, 1H), 7.25-7.02 (m, 3H), 5.54 (d, J=4.0 Hz, 1H), 5.46 (d, J=3.3 Hz, 2H), 3.75-3.55 (m, 1H), 3.53-3.44 (m, 0.5H), 3.43 (s, 3H), 3.30-3.11 (m, 3H), 3.03-2.79 (m, 3H), 2.60 (d, J=9.6 Hz, 0.5H), 2,52-2,40 (m, 1H), 2.14-1.97 (m, 1H), 1.93 (q, J=7.2 Hz, 2H), 1.85-1.45 (m, 3H), 1.32 (dd, J=17.5, 6.5 Hz, 3H), 1,28-1,20 (m, 6H), 1.05-0.91 (m, 3H), 0.72-0.57 (m, 3H) ppm. MS: M/e 501 (M+1)⁺.

### Compound A67: 2-(7-((2S,5R)-4-(1-(2,3-dihydrobenzofuran-5-yl)ethyl)-2,5-diethylpiperazin-1-yl)-4-methyl-5-oxo-4,5-dihydro-2H-pyrazolo[4,3-b]pyridin-2-yl)acetonitrile

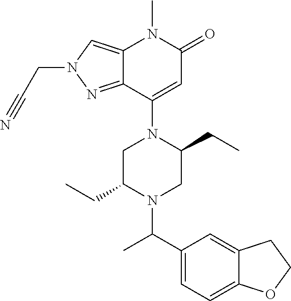 [SMILES: CC[C@H]1CN(C(C)c2ccc3c(c2)CCO3)[C@H](CC)CN1c1cc(=O)n(C)c2cn(CC#N)nc12]

### Step A: 1-(2.3-dihydrobenzofuran-5-yl)ethan-1-ol

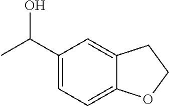

To a solution of 1-(2,3-dihydrobenzofuran-5-yl)ethan-1-one (320 mg, 2 mmol) in MeOH (10 mL) was added NaBH₄ (76 mg, 2 mmol) slowly at 0° C. The resulting mixture was stirred at room temperature for 1 hours. The reaction mixture was quenched with H₂O (20 mL) and extracted with EA (80 mL), washed with brine, dried over Na₂SO₄, filtered, and concentrated to dryness. The resulting residue was purified by flash column chromatography to give the desired product (280 mg, 85%).

### Step B: 2-(7-((2S,5R)-4-(1-(2.3-dihydrobenzofuran-5-yl)ethyl)-2,5-diethylpiperazin-1-yl-4-methyl-5-oxo-4,5-dihydro-2H-pyrazolo[4,3-b]pyridin-2-yl)acetonitrile

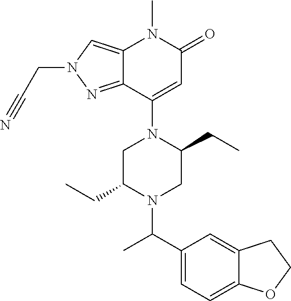 [SMILES: CC[C@H]1CN(C(C)c2ccc3c(c2)CCO3)[C@H](CC)CN1c1cc(=O)n(C)c2cn(CC#N)nc12]

To a solution of 1-(2,3-dihydrobenzofuran-5-yl)ethan-1-ol (33 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (75 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (70 mL), washed with brine, dried over Na₂SO₄, filtered, and concentrated to dryness. The resulting residue was purified by Prep-TLC (DCM/MeOH=20/1) and then Prep-HPLC (Method A) to give the titled compound (4 mg, 7%). ¹H NMR (400 MHz, CD₃OD) δ 7.92 (s, 1H), 7.30-7.18 (m, 1H), 7.14-7.00 (m, 1H), 6.74-6.62 (m, 1H), 5.55 (s, 1H), 5.47 (s, 2H), 4.60-4.45 (m, 2H), 3.80-3.45 (m, 2H), 3.43 (s, 3H), 3.35-3.31 (m, 1H), 3.25-3.08 (m, 3H), 3.06-2.80 (m, 1H), 2.71-2.32 (m, 2H), 2.21-1.42 (m, 4H), 1.40-1,25 (m, 3H), 1.06-0.90 (m, 3H), 0.76-0.60 (m, 3H) ppm. MS: M/e 475 (M+1)⁺.

### Compound A68: 2-(7-((2S,5R)-4-(1-(benzo[d][1,3]dioxol-5-yl)ethyl)-2,5-diethylpiperazin-1-yl)-4-methyl-5-oxo-4,5-dihydro-2H-pyrazolo[4,3-b]pyridin-2-yl)acetonitrile

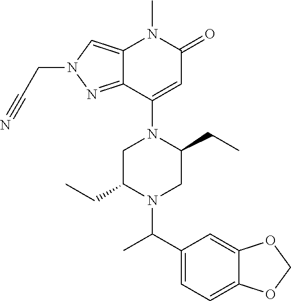 [SMILES: CC[C@H]1CN(C(C)c2ccc3c(c2)OCO3)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (300 mg, 0.91 mmol) in CH₃CN (3 mL) and was added 1-(benzo[d][1,3]dioxol-5-yl)ethan-1-ol (152 mg, 0.91 mmol), (cyanomethyl)trimethylphosphonium iodide (670 mg, 2.74 mmol) and DIPEA (590 mg, 4.57 mmol). The resulting mixture was stirred at 105° C. overnight. The reaction solvent was removed under reduced pressure to afford crude product. The resulting residue was purified Prep-HPLC (Method A) to give the titled Compound A68a (65 mg) and Compound A68b (55 mg).

Compound A68a (the earlier peak): ¹H NMR (400 MHz, CD₃OD) δ 7.91 (s, 1H), 6.88 (s, 1H), 6.77 (q, .=8.0 Hz, 2H), 5.93 (s, 2H), 5.55 (s, 1H), 5.46 (s, 2H), 3.66 (d, J=6.5 Hz, 1H), 3.43 (s, 3H), 3.26 (s, 3H), 2.98 (d, J=11.6 Hz, 1H), 2.84 (d, J=8.7 Hz, 1H), 2,47 (s, 1H), 2.15-2.04 (m, 1H), 1.85-1.76 (m, 1H), 1.54-1.46 (m, 2H), 1.31 (d, J=6.4 Hz, 3H), 0.95 (t, J=7.5 Hz, 3H), 0.71 (t, J=7.3 Hz, 3H). MS: M/e 477 (M+1)⁺.

Compound A68b (the later peak): ¹H NMR (400 MHz, CD₃OD) δ 7.92 (s, 1H), 6.92 (s, 1H), 6.82 (d, J=7.9 Hz, 1H), 6.73 (d, J=7.9 Hz, 1H), 5.91 (s, 2H), 5.54 (s, 1H), 5.47 (s, 2H), 3.54-3.45 (m, 2H), 3.43 (s, 3H), 3.30-3.27 (m, 2H), 3.11 (d, J=11.0 Hz, 1H), 2.63-2,55 (m, 1H), 2,43 (d, J=12.0 Hz, 1H), 2.02-1.92 (m, 1H), 1.68-1.58 (m, 2H), 1.56-1.47 (m, 1H), 1,27 (d, J=6.5 Hz, 3H), 1.01 (t, J=7.3 Hz, 3H), 0.66 (t, J=7.4 Hz, 3H). MS: M/e 477 (M+1)⁺.

### Compound A69: 2-(7-((2S,5R)-4-(1-(4-fluoro-2,2-dimethylbenzo[d][1,3]dioxol-5-yl)ethyl)-2,5-dimethylpiperazin-1-yl)-4-methyl-5-oxo-4,5-dihydro-2H-pyrazolo[4,3-b]pyridin-2-yl)acetonitrile

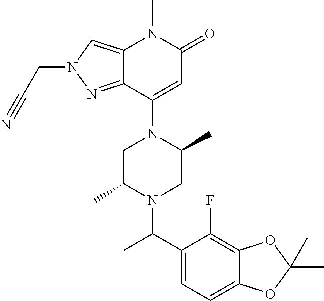 [SMILES: CC(c1ccc2c(c1F)OC(C)(C)O2)N1C[C@H](C)N(c2cc(=O)n(C)c3cn(CC#N)nc23)C[C@H]1C]

### Step A: 5-bromo-4-fluoro-2.2-dimethylbenzo[d][1,3]dioxole and 5-bromo-2.2-dimethylbenzo[d][1,3]dioxole

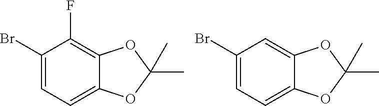

To a solution of 5-bromo-2,2-dimethylbenzo[d][1,3]dioxole (2.3 g, 10 mol) in THF (20 mL), cooled down to −70° C. and under N₂ atmosphere was added LDA (2M, 6.5 ml, 13 mol) by dropwise. The reaction mixture was stirred at −70° C. for 1 h. A solution of N-Fluorobenzenesulfonimide (3.78 g, 12 mmol) in THF (10 ml) was slowly added to the mixture and the reaction was slowly warmed up to RT. The mixture was added H₂O (20 ml) and extracted with EA (20 mL×3), then concentrated by using a rotary evaporator to give a residue. The resulting residue was purified by flash column chromatography to give a mixture of 5-bromo-4-fluoro-2,2-dimethylbenzo[d][1,3]dioxole and starting material 5-bromo-2,2-dimethylbenzo[d][1,3]dioxole (1.9 g). MS: M/e 247 (M+1)⁺ and 229 (M+1)⁺.

### Step B: 144-fluoro-2.2-dimethylbenzo[d][1,3]dioxol-5-yl)ethan-1-one and 142.2-dimethylbenzo[d][1,3]dioxol-5-yl)ethan-1-one

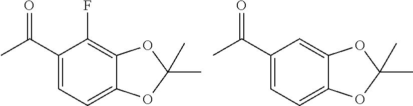

The mixture containing 5-bromo-4-fluoro-2,2-dimethylbenzo[d][1,3]dioxole and 5-bromo-2,2-dimethylbenzo[d][1,3]dioxole from last step (1.9 g, 7.7 mol) in toluene (15 mL) was added Pd(PPh₃)₂Cl₂ (269 mg, 0.38 mol) and tributyl(1-ethoxyvinyl)stannane (3.65 g, 10 mmol). The reaction mixture was protected by N₂ atmosphere and stirred at 90° C. for 16 hrs. The mixture was added H₂O (10 ml) and extracted with EA (20 mL×3). The organic phase was dried with Na₂SO₄ and concentrated by using a rotary evaporator to give a residue. The resulting residue was purified by flash column chromatography to give a mixture of 1-(4-fluoro-2,2-dimethylbenzo[d][1,3]dioxol-5-yl)ethan-1-one and 1-(2,2-dimethylbenzo[d][1,3]dioxol-5-yl)ethan-1-one (1.6 g). MS: M/e 211 (M+1)⁺ and 193 (M+1)⁺.

### Step C: 1-(4-fluoro-2,2-dimethylbenzo[d][1,3]dioxol-5-yl)ethan-1-ol and 142.2-dimethylbenzo[d][1,3]dioxol-5-yl)ethan-1-ol

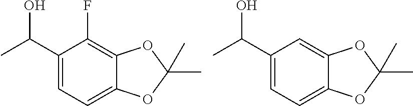

The mixture containing 1-(4-fluoro-2,2-dimethylbenzo[d][1,3]dioxol-5-yl)ethan-1-one and 1-(2,2-dimethylbenzo[d][1,3]dioxol-5-yl)ethan-1-one from last step (1.6 g, 7.6 mol) in MeOH was added NaBH₄ (290 mg, 7.6 mmol). The reaction mixture was stirred at RT for 15 mins. The mixture was added H₂O (30 ml) and extracted with DCM (30 mL×3). The organic phase was dried with Na₂SO₄ and concentrated by using a rotary evaporator to give a residue. The resulting residue was purified by flash column chromatography to give a mixture of 1-(4-fluoro-2,2-dimethylbenzo[d][1,3]dioxol-5-yl)ethan-1-ol and 1-(2,2-dimethylbenzo[d][1,3]dioxol-5-yl)ethan-1-ol (1.1 g). MS: M/e 213 (M+1)⁺ and 195 (M+1)⁺.

### Step D: 7-((2S,5R)-4-(1-(4-fluoro-2.2-dimethylbenzo[d][1,3]dioxol-5-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one and 7-((2S,5R)-4-(1-(2.2-dimethylbenzo[d][1,3]dioxol-5-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one

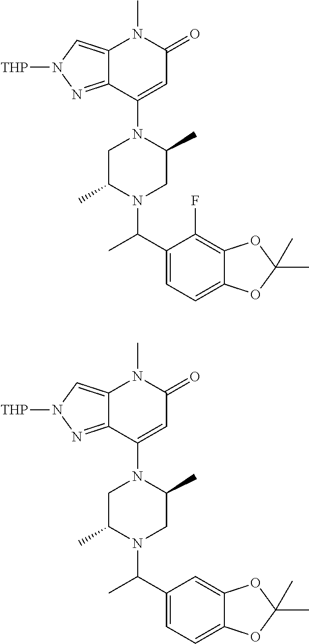

To a solution of 7-((2S,5R)-2,5-dimethylpiperazin-1-yl)-4-methyl-2-(tetrahydro-2H-pyran-2-yl)-2,4-dihydro-5H-pyrazolo[4,3-b]pyridin-5-one (345 mg, 1 mmol), the mixture containing 1-(4-fluoro-2,2-dimethylbenzo[d][1,3]dioxol-5-yl)ethan-1-ol and 1-(2,2-dimethylbenzo[d][1,3]dioxol-5-yl)ethan-1-ol (300 mg, 1.5 mmol) from last step and (cyanomethyl)trimethylphosphonium iodide (482 mg, 2 mmol) in CH₃CN (10 mL) was added DIPEA (516 mg, 4 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by flash column chromatography to give a mixture of 7-((2S,5R)-4-(1-(4-fluoro-2,2-dimethylbenzo[d][1,3]dioxol-5-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one and 7-((2S,5R)-4-(1-(2,2-dimethylbenzo[d][1,3]dioxol-5-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (100 mg, 18.5%). MS: M/e 540 (M+1)⁺ and 522 (M+1)⁺.

### Step E: 7-((2S,5R)-4-(1-(4-fluoro-2.2-dimethylbenzo[d][1,3]dioxol-5-yl)ethyl)-2,5-dimethylpiperazine-1-yl)-4-methyl-2,4-dihydro-5H-pyrazolo[4,3-b]pyridin-5-one and 7-((2S,5R)₄-(1-(2.2-dimethylbenzo[d][1,3]dioxol-5-yl)ethyl)-2,5-dimethylpiperazin-1-yl)-4-methyl-2,4-dihydro-5H-pyrazolo[4,3-b]pyridin-5-one

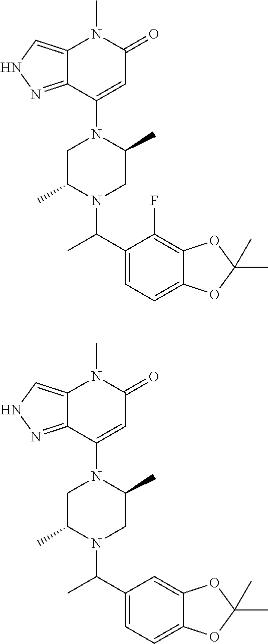 [SMILES: CC(c1ccc2c(c1F)OC(C)(C)O2)N1C[C@H](C)N(c2cc(=O)n(C)c3c[nH]nc23)C[C@H]1C ; CC(c1ccc2c(c1)OC(C)(C)O2)N1C[C@H](C)N(c2cc(=O)n(C)c3c[nH]nc23)C[C@H]1C]

The mixture containing 7-((2S,5R)-4-(1-(4-fluoro-2,2-dimethylbenzo[d][1,3]dioxol-5-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one and 7-((2S,5R)-4-(1-(2,2-dimethylbenzo[d][1,3]dioxol-5-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one from last step (26 mg, 0.048 mol) in MeCN (10 ml) was added TMSOTf (10.7 mg, 0.048 mmol). The reaction mixture was stirred at RT for 1 h. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by Prep-HPLC (Method A) to give 7-((2S,5R)-4-(1-(4-fluoro-2,2-dimethylbenzo[d][1,3]dioxol-5-yl)ethyl)-2,5-dimethylpiperazin-1-yl)-4-methyl-2,4-dihydro-5H-pyrazolo[4,3-b]pyridin-5-one (10 mg), MS: M/e 456 (M+1)⁺ and 7-((2S,5R)-4-(1-(2,2-dimethylbenzo[d][1,3]dioxol-5-yl)ethyl)-2,5-dimethylpiperazin-1-yl)-4-methyl-2,4-dihydro-5H-pyrazolo[4,3-b]pyridin-5-one (7 mg), MS:438 (M+1)⁺.

### Step F: 2-(7-((2S,5R)-4-(1-(4-fluoro-2.2-dimethylbenzo[d][1,3]dioxol-5-yl)ethyl)-2,5-dimethylpiperazin-1-yl)-4-methyl-5-oxo-4,5-dihydro-2H-pyrazolo[4,3-b]pyridin-2-yl)acetonitrile

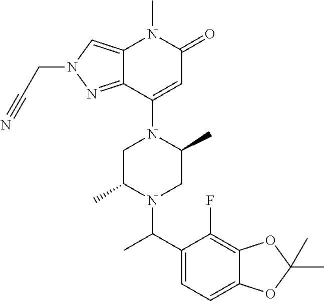 [SMILES: CC(c1ccc2c(c1F)OC(C)(C)O2)N1C[C@H](C)N(c2cc(=O)n(C)c3cn(CC#N)nc23)C[C@H]1C]

To a solution of 7-((2S,5R)-4-(1-(4-fluoro-2,2-dimethylbenzo[d][1,3]dioxol-5-yl)ethyl)-2,5-dimethylpiperazin-1-yl)-4-methyl-2,4-dihydro-5H-pyrazolo[4,3-b]pyridin-5-one (10 mg, 0.02 mol) in DMF (3 ml) was added K₂CO₃ (13.9 mg, 0.1 mmol) and 2-chloroacetonitrile (7.5 mg, 0.1 mmol). The reaction mixture was stirred at RT for overnight. The mixture was added H₂O (10 ml) and extracted with EA (20 mL×3). The organic phase was dried with Na₂SO₄ and concentrated by using a rotary evaporator to give a residue. The resulting residue was purified by Prep-HPLC (Method A) to give the titled compound(0.83 mg, 8.9%). ¹H NMR (400 MHz, CD₃OD) δ 7.92 (d, J=1.9 Hz, 1H), 6.96 (m, 1H), 6.58 (dd, J=12,4, 8.2 Hz, 1H), 5.56 (d, J=3.9 Hz, 1H), 5.47 (d, J=1.5 Hz, 2H), 3.97 (d, J=6.5 Hz, 0.5H), 3.83 (d, J=6.9 Hz, 0.5H), 3.58 (m, 1H), 3.42 (d, J=18.5 Hz, 5H), 3.16 (s, 1H), 3.00 (dd, J=11.9, 4.5 Hz, 1H), 2.78 (d, J=12,4 Hz, 1H), 1.69 (t, J=4.4 Hz, 6H), 1.35 (d, J=6.5 Hz, 3H), 1.32 (s, 1.5H), 1,23 (d, J=6.6 Hz, 1.5H), 1.13 (d, J=6.4 Hz, 1.5H), 1.01 (d, J=6.5 Hz, 1.5H) ppm. MS: M/e 495 (M+1)⁺.

### Compound A70: 2-(7-((2S,5R)-4-(1-(2,2-dimethylbenzo[d][1,3]dioxol-5-yl)ethyl)-2,5-dimethylpiperazin-1-yl)-4-methyl-5-oxo-4,5-dihydro-2H-pyrazolo[4,3-b]pyridin-2-yl)acetonitrile

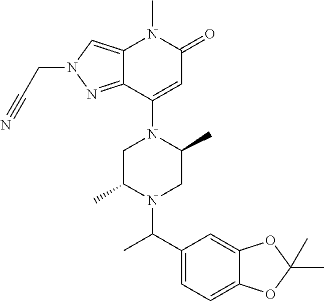 [SMILES: CC(c1ccc2c(c1)OC(C)(C)O2)N1C[C@H](C)N(c2cc(=O)n(C)c3cn(CC#N)nc23)C[C@H]1C]

To a solution of 7-((2S,5R)-4-(1-(2,2-dimethylbenzo[d][1,3]dioxol-5-yl)ethyl)-2,5-dimethylpiperazin-1-yl)-4-methyl-2,4-dihydro-5H-pyrazolo[4,3-b]pyridin-5-one (7 mg, 0.016 mol) in DMF (3 ml) was added K₂CO₃ (13.9 mg, 0.1 mmol) and 2-chloroacetonitrile (7.5 mg, 0.1 mmol). The reaction mixture was stirred at RT for overnight. The mixture was added saturated H₂O (10 ml) and extracted with EA (20 mL×3). The organic phase was dried with Na₂SO₄ and concentrated by using a rotary evaporator to give a residue. The resulting residue was purified by Prep-HPLC (Method A) to give S-1082 (1,2 mg, 15.7%). S 7.92 (d, J=1.3 Hz, 1H), 6.82 (s, 1H), 6.76 (m, 1H), 6.64 (dd, J=12.1, 7.8 Hz, 1H), 5.56 (d, J=2.7 Hz, 1H), 5.47 (d, J=2.1 Hz, 2H), 3.61 (m, 2H), 3.42 (d, J=11.8 Hz, 5H), 3.16 (s, 0.5H), 2.97 (s, 1H), 2.84 (d, J=13.2 Hz, 0.5H), 2.73 (s, 0.5H), 2.32 (s, 0.5H), 1.64 (m, 6H), 1.38 (d, J=6.6 Hz, 1.5H), 1.30 (dd, J=13.0, 6.2 Hz, 3H), 1,22 (d, J=6.6 Hz, 1.5H), 1.13 (d, J=6.3 Hz, 1.5H), 0.99 (d, J=6.4 Hz, 1.5H) ppm. MS: M/e 477 (M+1)⁺.

### Compound A71: 2-(7-((2S,5R)-2,5-diethyl-4-(1-(4-fluoro-2,2-dimethylbenzo[d][1,3]dioxol-5-yl)ethyl)piperazin-1-yl)-4-methyl-5-oxo-4,5-dihydro-2H-pyrazolo[4,3-b]pyridin-2-yl)acetonitrile

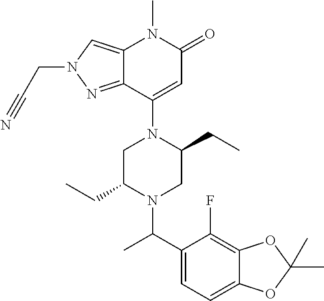 [SMILES: CC[C@H]1CN(C(C)c2ccc3c(c2F)OC(C)(C)O3)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(4-fluoro-2,2-dimethylbenzo[d][1,3]dioxol-5-yl)ethan-1-ol (50 mg, 0.25 mmol) and (cyanomethyl)trimethylphosphonium iodide (120 mg, 0.5 mmol) in CH₃CN (2 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method A) to give the titled compound (3 mg, 6%) ¹H NMR (500 MHz, CD₃OD) δ 7.92 (d, J=2.6 Hz, 1H), 6.93 (m, 1H), 6.58 (dd, J=14.3, 8.2 Hz, 1H), 5.55 (s, 1H), 5.47 (d, J=2.9 Hz, 2H), 4.05 (q, J=6.6 Hz, 0.5H), 3.90 (d, J=6.6 Hz, 0.5H), 3.45 (m, 4H), 3.32 (s, 1H), 3.10 (d, J=10.1 Hz, 0.5H), 2.90 (s, 1H), 2.65 (s, 0.5H), 2,47 (m, 1H), 2.01 (m, 1H), 1.77 (s, 1H), 1.68 (d, J=9.0 Hz, 6H), 1.63 (dd, J=7.0, 4.1 Hz, 1H), 1.51 (m, 2H), 1.36 (d, J=6.6 Hz, 1.5H), 1.32 (d, J=6.6 Hz, 1.5H), 1.01 (t, .=7.3 Hz, 1.5H), 0.93 (t, J=7.4 Hz, 1.5H), 0.77 (t, J=7.3 Hz, 1.5H), 0.68 (t, J=7.3 Hz, 1.5H) ppm. MS: M/e 523 (M+1)⁺.

### Compound A72: 2-(7-((2S,5R)-4-(1-(6,7-dihydro-5H-cyclopenta[b]pyridin-2-yl)ethyl)-2,5-diethylpiperazin-1-yl)-4-methyl-5-oxo-4,5-dihydro-2H-pyrazolo[4,3-b]pyridin-2-yl)acetonitrile

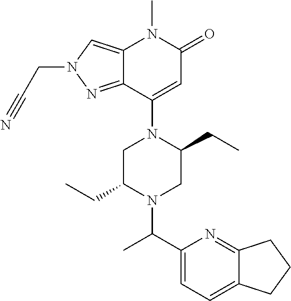 [SMILES: CC[C@H]1CN(C(C)c2ccc3c(n2)CCC3)[C@H](CC)CN1c1cc(=O)n(C)c2cn(CC#N)nc12]

### Step A: 1-(6.7-dihydro-5H-cyclopenta[b]pyridin-2-yl)ethan-1-one

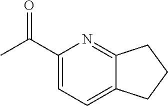

A mixture of 2-chloro-6,7-dihydro-5H-cyclopenta[b]pyridine (460 mg, 3 mmol), tributyl(1-ethoxyvinyl)stannane (1.6 g, 4.5 mmol) and Pd(PPh₃)₂Cl₂ (210 mg, 0.3 mmol) in toluene (5 mL) was stirred at 100° C. under N₂ for 16 hours. To the resulting solution was added HCl (1 mL, con.) in drops and the mixture was stirred at room temperature for 1 hour. The mixture was treated with saturated NaHCO₃ aq. (20 ml), extracted with EtOAc(10 ml×3), washed with brine (20 mL), dried and concentrated to dryness. The resulting residue was purified by flash column chromatography (EtOAc: PE=0-50% in 20 minutes) to give the titled compound (100 mg, 21%). MS: M/e 162 (M+1)⁺.

### Step B: 1-(6.7-dihydro-5H-cyclopenta[b]pyridin-2-yl)ethan-1-ol

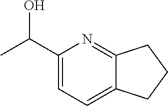

To a solution of 1-(6,7-dihydro-5H-cyclopenta[b]pyridin-2-yl)ethan-1-one (100 mg, 0.62 mmol) in MeOH (4 mL) was added NaBH₄ (19 mg, 0.5 mmol) at room temperature and the mixture was stirred at room temperature for 30 minutes. The resulting mixture was treated with water (30 ml), extracted with EtOAc (10 mL×2). The combined organic layers were dried over Na₂SO₄ and concentrated to dryness. The resulting residue (90 mg, crude) was used in the next step. MS: M/e 164 (M+1)⁺.

### Step C: 2-(7-((2S,5R)-4-(1-(6.7-dihydro-5H-cyclopenta[b]pyridin-2-yl)ethyl)-2,5-diethylpiperazin-1-yl-4-methyl-5-oxo-4,5-dihydro-2H-pyrazolo[4,3-b]pyridin-2-yl)acetonitrile

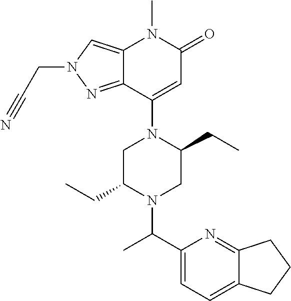 [SMILES: CC[C@H]1CN(C(C)c2ccc3c(n2)CCC3)[C@H](CC)CN1c1cc(=O)n(C)c2cn(CC#N)nc12]

A mixture of 1-(6,7-dihydro-5H-cyclopenta[b]pyridin-2-yl)ethan-1-ol (32 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (97 mg, 0.4 mmol) and DIPEA (52 mg, 0.4 mmol) in MeCN (2 mL) was stirred at 100° C. overnight. The reaction mixture was diluted with EtOAc (20 mL), washed with water (10 mL×2), dried over Na₂SO₄ and concentrated to dryness. The resulting residue was purified by Prep-TLC (MeOH: DCM=1:15) to give the titled compound (19 mg, 40%). ¹H NMR (400 MHz, DMSO-d⁶) δ 7.96 (d, J=2.6 Hz, 1H), 7.56 (d, J=7.9 Hz, 1H), 7.21 (dd, J=19.9, 7.8 Hz, 1H), 5.61 (d, J=11.3 Hz, 2H), 5.36 (s, 1H), 3.82-3.57 (m, 1H), 3.36 (s, 1H), 3.25 (s, 3H), 3.15-2.96 (m, 1H), 2.89-2,51 (m, 6H), 2.34-2.16 (m, 1H), 2.06-1.83 (m, 3H), 1.67-1.35 (m, 4H), 1,28-1.19 (m, 3H), 0.93-0.80 (m, 3H), 0.66-0.53 (m, 3H) ppm. MS: M/e 474 (M+1)⁺.

### Compound A73: 2-(7-((2S,5R)-4-(1-(2,3-dihydrofuro[3,2-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile

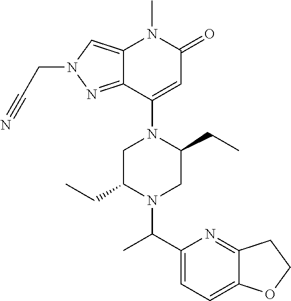 [SMILES: CC[C@H]1CN(C(C)c2ccc3c(n2)CCO3)[C@H](CC)CN1c1cc(=O)n(C)c2cn(CC#N)nc12]

### Step A: 2.3-dihydrofuro[3,2-b]pyridine

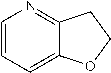

A mixture of furo[3,2-b]pyridine (1 g, 8.4 mmol) and Pd/C(0.3 g) in MeOH(15 mL) at room temperature was added AcOH (0.5 mL). The resulting mixture was degassed 3 times under H₂ atmosphere, and stirred at room temperature under H₂ (0.4 MPa) atmosphere for 16 hours. After filtration, the combined organic layers were concentrated under reduced pressure to give the crude product. The crude product was basified by Na₂CO₃ (4M) to pH ˜10 and extracted with EA. The resulting residue was concentrated to dryness. The resulting residue was purified by flash column chromatography to give the titled compound (500 mg, 49%). MS: M/e 122 (M+1)⁺.

### Step B: 2.3-dihydrofuro[3,2-b]pyridine 4-oxide

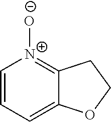

To a solution of 2,3-dihydrofuro[3,2-b]pyridine (0.5 g, 4 mmol) in DCM (20 mL) was added 3-chlorobenzoperoxoic acid (2.1 g, 12.2 mmol). The mixture solution was stirred at room temperature for two days. The reaction was concentrated to dryness. The resulting residue was purified by flash column chromatography to give the titled compound (0.4 g, 70%). MS: M/e 138 (M+1)⁺.

### Step C: 2.3-dihydrofuro[3,2-b]pyridine-5-carbonitrile

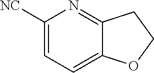 [SMILES: N#Cc1ccc2c(n1)CCO2]

To a solution of 2,3-dihydrofuro[3,2-b]pyridine 4-oxide (0.4 g, 2.8 mmol) in CH₃CN (8 mL) were added TMSCN (860 mg, 8.6 mmol), Et₃N (1.17 g, 11.5 mol). The mixture was stirred at 90° C. for overnight. The reaction was concentrated to dryness. The resulting residue was purified by flash column chromatography to give the titled compound (120 mg, 28%). MS: We 147 (M+1)⁺.

### Step D: 1-(2.3-dihydrofuro[3,2-b]pyridin-5-yl)ethan-1-one

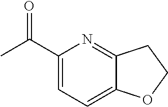

A mixture of 2,3-dihydrofuro[3,2-b]pyridine-5-carbonitrile (120 mg, 0.81 mmol) in THF (5 mL) at 0° C. was added methyl magnesium bromide (0.55 mL, 3M, 1.64 mol). The reaction mixture was stirred at room temperature for 1 hour. The reaction was quenched by adding saturated solution of NH₄Cl. The resulting mixture was extracted with EA, and then concentrated by using a rotary evaporator, to give the titled compound (100 mg). MS: M/e 164 (M+1)⁺.

### Step E: 142.3-dihydrofuro[3,2-bipyridin-5-yl)ethan-1-ol

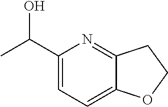

To a solution of 1-(2,3-dihydrofuro[3,2-b]pyridin-5-yl)ethan-1-one (100 mg, 0.6 mmol) in MeOH (5 mL) was added NaBH₄ (23 mg, 0.6 mmol) at room temperature and the resulting mixture was stirred at room temperature for 5 min. The reaction mixture was diluted with DCM and washed with water, dried over Na₂SO₄ and concentrated to give the titled compound (100 mg). MS: M/e 166 (M+1)⁺.

### Step F: 2-(7-((2S,5R)-4-(1-(2.3-dihydrofuro[3,2-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile

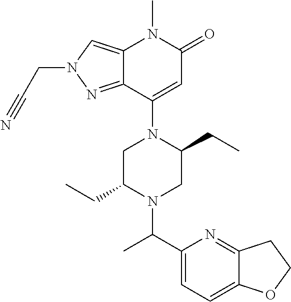 [SMILES: CC[C@H]1CN(C(C)c2ccc3c(n2)CCO3)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (2 mL) was added 1-(2,3-dihydrofuro[3,2-b]pyridin-5-yl)ethan-1-ol (63 mg, 0.38 mmol), (cyanomethyl)trimethylphosphonium iodide (110 mg, 0.45 mmol) and DIPEA(197 mg, 1.5 mmol).The resulting mixture was stirred at 105° C. overnight. The reaction solvent was removed under reduced pressure. The reaction was diluted with DCM and washed with water. The organic layer was separated, dried by Na₂SO₄, filtered, and concentrated to dryness, then purified by flash column chromatography (DCM:MeOH) to give the titled Compound (crude), which was further purified to give the titled compound a (5 mg) by Prep-HPLC (Method A). ¹H NMR (400 MHz, CD₃OD) δ 7.91 (d, J=3.6 Hz, 1H), 7.27 (dd, J=17.2, 8.4 Hz, 1H), 7.11 (dd, J=8.3, 2.3 Hz, 1H), 5.54 (d, J=3.3 Hz, 1H), 5.45 (d, J=3.5 Hz, 2H), 4.87 (s, 1H), 4.66 (t, J=8.9 Hz, 2H), 4.04-3.46 (m, 2H), 3.42 (s, 3H), 3.31 (d, J=6.0 Hz, 2H), 3.26-2.69 (m, 3H), 2,46 (s, 1H), 2.20-1.48 (m, 4H), 1.41-1,28 (m, 3H), 1.09-0.85 (m, 3H), 0.79-0.60 (m, 3H) ppm. MS: M/e 476 (M+1)⁺.

### Compound A74: 2-(7-((2S,5R)-4-(1-(2,3-dihydrofuro[2,3-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile

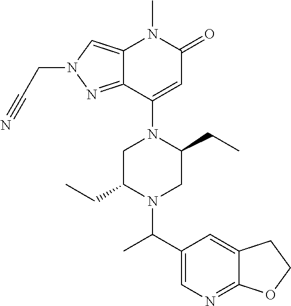 [SMILES: CC[C@H]1CN(C(C)c2cnc3c(c2)CCO3)[C@H](CC)CN1c1cc(=O)n(C)c2cn(CC#N)nc12]

### Step A: 5-bromo-2,3-dihydrofuro[2,3b]pyridine

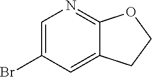

To a solution of 2,3-dihydrofuro[2,3-b]pyridine (300 mg, 2,4 mmol) in CH₃CN (3 mL) was added NBS (662 mg, 3.7 mmol) at room temperature and the resulting mixture was stirred at 60° C. under N₂ for 16 hours. The resulting residue was purified by flash column chromatography to give the titled compound (220 mg, 45%). MS: M/e 200 (M+1)⁺

### Step B: 1423-dihydrofuro[2.3-b]pyridin-5-yl)ethan-1-one

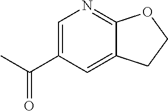

A mixture of 5-bromo-2,3-dihydrofuro[2,3-b]pyridine (220 mg, 1.3 mmol), tributyl(1-ethoxyvinyl)stannane (0.7 g, 2 mmol) and Pd(PPh₃)₂Cl₂ (70 mg, 0.13 mmol) in toluene (10 mL) was stirred at 100° C. under N₂ for 16 hours. The reaction mixture was diluted with HCl (2 mL, 3 M in ethyl acetate) in drops and the mixture was stirred at room temperature for 2 hours. The reaction was concentrated and adjusted to pH=8-9 with saturated NaHCO₃ solution, extracted with DCM (20 mL×2). The combined organic layers were washed with brine, dried over Na₂SO₄ and concentrated to dryness. The resulting residue was purified by flash column chromatography to give the titled compound (70 mg, 37%). MS: We 164 (M+1)⁺

### Step C: 1-(2.3-dihydrofuro[2,3-b]pyridin-5-yl)ethan-1-ol

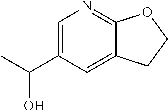

To a solution of 1-(2,3-dihydrofuro[2,3-b]pyridin-5-yl)ethan-1-one (70 mg, 0.42 mmol) in MeOH (3 mL) was added NaBH₄ (16 mg, 0.42 mmol) at room temperature and the resulting mixture was stirred at room temperature for 5 min. The reaction mixture was diluted with DCM and washed with water, dried over Na₂SO₄ and concentrated to give the titled compound (50 mg). MS: M/e 166 (M+1)⁺.

### Step D: 2-(7-((2S,5R_{1.4}-(1 (2.3-dihydrofuro[2,3-b]pyridin-5-yl)ethyl-2,5-diethylpiperazin-1-yl)-4-methyl-5-oxo-4,5-dihydro-2H-pyrazolo[4,3-b]pyridin-2-yl)acetonitrile

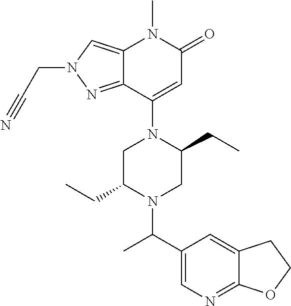 [SMILES: CC[C@H]1CN(C(C)c2cnc3c(c2)CCO3)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.09 mmol) in CH₃CN (2 mL) and was added 1-(2,3-dihydrofuro[2,3-b]pyridin-5-yl)ethan-1-ol (45 mg, 0.18 mmol), (cyanomethyl)trimethylphosphonium iodide (67 mg, 0.27 mmol) and DIPEA(117 mg, 0.9 mmol).The resulting mixture was stirred at 105° C. overnight. The reaction solvent was removed under reduced pressure. The resulting residue was purified by flash column chromatography to give the titled compound(crude), which was further purified to give the titled compound (1 mg) by Prep-HPLC (Method A). ¹H NMR (400 MHz, CD₃OD) δ 7.92 (d, J=2.0 Hz, 1H), 7.84-7.69 (m, 2H), 5.55 (s, 1H), 5.47 (d, J=3.2 Hz, 2H), 4.78-4.05 (m, 3H), 3.83-3.46 (m, 2H), 3.43 (s, 3H), 3.26-3.21 (m, 3H), 3.19-2,58 (m, 2H), 2.39 (d, J=12.6 Hz, 1H), 2.15-1.45 (m, 4H), 1.33 (dd, J=12.0, 6.5 Hz, 3H), 0.99 (dt, J=25.3, 7.4 Hz, 3H), 0.70 (dt, J=30.7, 7.5 Hz, 3H) ppm. MS: M/e 476 (M+1)⁺.

### Compound A75: 2-(7-((2S,5R)-4-(1-([1,3]dioxolo[4,5-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile

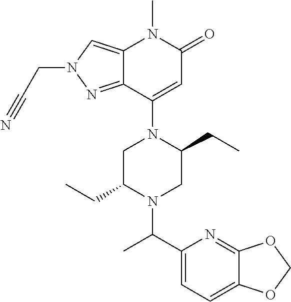 [SMILES: CC[C@H]1CN(C(C)c2ccc3c(n2)OCO3)[C@H](CC)CN1c1cc(=O)n(C)c2cn(CC#N)nc12]

### Step A: [1,3]dioxolo[4.5-b]pyridine

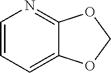

A mixture of pyridine-2,3-diol (5.55 g, 50 mmol), dibromomethane (17.4 g, 100 mmol) and K₂CO₃ (13.8 g, 100 mmol) in DMSO (70 mL) was stirred at 80° C. for 16 hours. The mixture was cooled and treated with EA (100 mL) and H₂O (150 mL) before filtered through a celite pad. Layers were separated and the aqueous layer was extracted with EA (50 mL×3). The combined organics was washed with brine (100 mL×3), dried over Na₂SO₄, and concentrated to dryness. The resulting residue was purified by column chromatography to give the titled compound (1.53 g, 25%). ¹H NMR (500 MHz, CDCl₃) δ 7.65 (dd, J=5.5, 1.0 Hz, 1H), 6.99 (dd, J=7.5, 1.0 Hz, 1H), 6.76 (dd, J=7.5, 5.5 Hz, 1H), 6.05 (s, 2H).

### Step B: 5-bromo-[1,3]dioxolo[4.5-b]pyridine

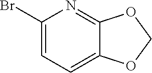

To a mixture of [1,3]dioxolo[4,5-b]pyridine (1.4 g, 11.4 mmol) and AcONa (4.7 g, 57 mmol) in AcOH (20 mL) was added a solution of Br₂/AcOH (250 mg/mL, 7.3 mL×3) at 50° C. with 16 hours. The mixture was cooled and the suspension was filtered and the filtrate was concentrated to remove the excess of AcOH and resulted a 5 mL of oil which was diluted with EA (50 mL), basified with NaHCO₃ to pH-8. The resulted mixture was washed brine (20 mL×3), dried over Na₂SO₄, and concentrated to dryness. The resulting residue was purified by column chromatography to give the titled compound (750 mg, crude). ¹H NMR (400 MHz, CDCl₃) δ 6.94 (d, J=7.6 Hz, 1H), 6.87 (d, J=8.0 Hz, 1H), 6.07 (s, 2H).

### Step C: 1-([1,3]dioxolo[4.5-b]pyridin-5-yl)ethan-1-one

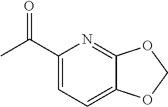

A mixture of 5-bromo-[1,3]dioxolo[4,5-b]pyridine (640 mg, 3.17 mmol), tributyl(1-ethoxyvinyl)stannane (2.28 g, 6.33 mmol) and Pd(PPh₃)₂Cl₂ (222 mg, 0.32 mmol) in toluene (12 mL) was stirred at 100° C. under N₂ for 16 hrs. The mixture was cooled and diluted with EA (20 mL), washed with brine (10 mL×3), dried over Na₂SO₄, filtered and the filtrate was added a solution of HCl/Dioxane (4M, 10 mL) and stirred for 10 minutes. The resulted mixture was washed with brine (10 mL×2), NaHCO₃ (10 mL×2), dried over Na₂SO₄, filtered and concentrated to dryness. The resulting residue was purified by column chromatography to give the titled compound (160 mg, 30%). ¹H NMR (500 MHz, CDCl₃) δ 7.70 (d, J=8.0 Hz, 1H), 7.08 (d, J=8.0 Hz, 1H), 6.16 (s, 2H), 2.62 (s, 3H).

### Step D: 1-([1,3]dioxolo[4.5-b]pyridin-5-yl)ethan-1-ol

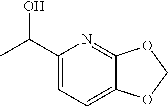

To a solution of 1-([1,3]dioxolo[4,5-b]pyridin-5-yl)ethan-1-one (160 mg, 0.97 mmol) in MeOH (3 mL) was added NaBH₄ (38 g, 1.0 mmol) in portions at RT and the mixture was stirred at RT for 30 minutes. The mixture was diluted with EtOAc (10 mL), washed with brine (5 mL×2), dried over Na₂SO₄ and concentrated to dryness. The resulted residue was purified by column chromatography to give the titled compound (120 mg, 74%). ¹H NMR (500 MHz, DMSO-d₆) δ 7.19 (d, J=8.0 Hz, 1H), 6.92 (d, J=8.0 Hz, 1H), 6.10 (s, 1H), 6.09 (s, 1H), 5.21 (d, J=5.0 Hz, 1H), 4.63-4.45 (m, 1H), 1.30 (d, J=6.5 Hz, 3H).

### Step E: 2-(7-((2S,5R)-4-(1-([1,3]dioxolo[4.5-b]pyridin-5-yl)ethyl)-2,5-diethylpiperazin-1-yl-4-methyl-5-oxo-4,5-dihydro-2H-pyrazolo[4,3-b]pyridin-2-yl)acetonitrile

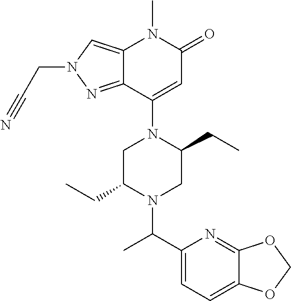 [SMILES: CC[C@H]1CN(C(C)c2ccc3c(n2)OCO3)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (59 mg, 0.18 mmol), 1-([1,3]dioxolo[4,5-b]pyridin-5-yl)ethan-1-ol (60 mg, 0.36 mmol) and (cyanomethyl)trimethylphosphonium iodide (153 mg, 0.63 mmol) in CH₃CN (1 mL) was added DIPEA (140 mg, 1.08 mmol). The mixture was stirred at 100° C. in a sealed tube for 16 hours. The mixture was cooled and diluted with EtOAc (5 mL), washed with brine (2 mL×3), dried over Na₂SO₄, filtered and concentrated under reduced pressure. The resulting residue was purified by Prep-TLC and further separated into Compound A75a (4 mg) and Compound A75b (4 mg) by Prep-Chiral-HPLC. The chiral separation conditions are shown below.

| Column | IE |
|---|---|
| Column Size | 20.0 mm × 250 mm, 5 um |
| Mobile Phase A | HEXANE 60% |
| Mobile Phase B | ETOH(0.2% 2M NH₃MEOH) 40% |
| Run Time | 15 min |
| Flow Rate | 18 mL/min |
| Wave Length | UV 210 nm and 310 nm |
| Temperature | RT |
| Prep-HPLC Equipment | Prep-HPLC-Gilson |

Compound A75a (the earlier peak): ¹H NMR (500 MHz, CD₃OD) δ 7.91 (s, 1H), 7.12 (d, J=7.5 Hz, 1H), 6.95 (d, J=7.5 Hz, 1H), 6.10 (s, 2H), 5.55 (s, 1H), 5.46 (s, 2H), 4.70-4.26 (m, 1H), 3.75 (q, J=6.5 Hz, 1H), 3.43 (s, 3H), 3.39-3.32 (m, 2H), 2.98-2.87 (m, 2H), 2,55-2,42 (m, 1H), 2.13-1.94 (m, 1H), 1.87-1.73 (m, 1H), 1.58-1.48 (m, 2H), 1.34 (d, J=6.5 Hz, 3H), 0.94 (t, J=7.5 Hz, 3H), 0.76 (t, J=7.5 Hz, 3H). MS: M/e 478 (M+1)⁺.

Compound A75b (the later peak): ¹H NMR (500 MHz, CD₃OD) 5 7.92 (s, 1H), 7.12 (d, J=7.8 Hz, 1H), 7.01 (d, J=7.8 Hz, 1H), 6.09 (d, J=3.5 Hz, 2H), 5.55 (s, 1H), 5.47 (s, 2H), 4.58-3.98 (m, 1H), 3.57 (q, J=6.5 Hz, 1H), 3.50 (d, J=13.5 Hz, 1H), 3.43 (s, 3H), 3.39-3.32 (m, 1H), 3.08 (d, J=10.0 Hz, 1H), 2.71 (dd, J=12.0, 3.0 Hz, 1H), 2,40 (d, J=12.0 Hz, 1H), 2.10-1.89 (m, 1H), 1.74-1.55 (m, 2H), 1.55-1.43 (m, 1H), 1.30 (d, J=6.5 Hz, 3H), 1.00 (t, J=7.5 Hz, 3H), 0.70 (t, J=7.5 Hz, 3H). MS: M/e 478 (M+1)⁺.

### Compound A76: 2-(7-((2S,5R)-2,5-diethyl-4-(1-(3-fluoropyrazolo[1,5-a]pyridin-6-yl)ethyl)piperazin-1-yl)-4-methyl-5-oxo-4,5-dihydro-2H-pyrazolo[4,3-b]pyridin-2-yl)acetonitrile

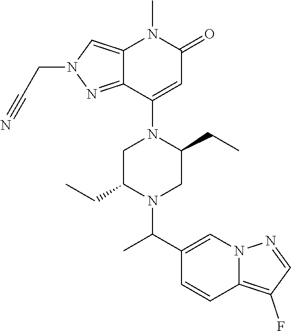 [SMILES: CC[C@H]1CN(C(C)c2ccc3c(F)cnn3c2)[C@H](CC)CN1c1cc(=O)n(C)c2cn(CC#N)nc12]

### Step A: 6-bromo-3-fluoropyrazolo[1.5-a]pyridine

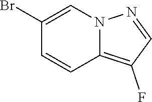 [SMILES: Fc1cnn2cc(Br)ccc12]

To a solution of 6-bromopyrazolo[1,5-a]pyridine (0.6 g, 3 mol) in MeCN (20 mL) was added Select F (2.7 g, 6 mol). The reaction mixture was protected by N₂ atmosphere and stirred at RT for 4 hrs. The mixture was added H₂O (20 ml) and extracted with EA (20 mL×3), then concentrated by using a rotary evaporator to give a residue. The resulting residue was purified by flash column chromatography to give the titled compound (350 mg, 54.5%). MS: M/e 215 (M+1)⁺.

### Step B: 1-(3-fluoropyrazolo[1,5-a]pyridin-6-yl)ethan-1-one

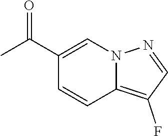

To a solution of 6-bromo-3-fluoropyrazolo[1,5-a]pyridine (350 mg, 1.6 mol) in toluene (10 mL) was added Pd(PPh₃)₂Cl₂ (140 mg, 0.2 mol) and tributyl(1-ethoxyvinyl)stannane (1.1 g, 3.2 mmol). The reaction mixture was protected by N₂ atmosphere and stirred at 90° C. for 16 hrs. The mixture was added 4N HCl in dioxane (0.5 ml) and stirred at RT for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (80 mg, 28.1%). MS: M/e 179 (M+1)⁺.

### Step C: 1-(3-fluoropyrazolo[1,5-a]pyridin-6-yl)ethan-1-ol

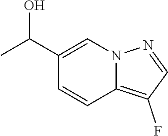

To a solution of 1-(3-fluoropyrazolo[1,5-a]pyridin-6-yl)ethan-1-one (40 mg, 0.2 mol) in MeOH was added NaBH₄ (38 mg, 1 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (15 mg, 37.5%). MS: M/e 181 (M+1)⁺.

### Step D: 2-(7-((2S,5R)-2,5-diethyl-4-(1-(3-fluoropyrazolo[1,5-a]pyridin-6-yl)ethyl)piperazin-1-yl)-4-methyl-5-oxo-4,5-dihydro-2H-pyrazolo[4,3-b]pyridin-2-yl)acetonitrile

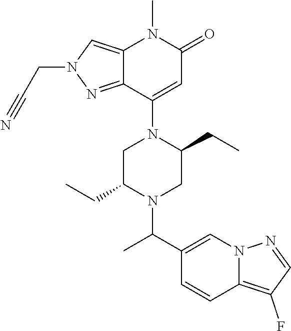 [SMILES: CC[C@H]1CN(C(C)c2ccc3c(F)cnn3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(3-fluoropyrazolo[1,5-a]pyridin-6-yl)ethan-1-ol (15 mg, 0.08 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method A) to give the titled compound (0.3 mg, 6.9%) as a formic acid salt. ¹H NMR (400 MHz, CD₃OD) δ 8.51 (s, 1H), 8.35 (d, J=6.3 Hz, 1H), 7.92 (d, J=2.8 Hz, 1H), 7.85 (m, 1H), 7.60 (dd, J=14.4, 9.3 Hz, 1H), 7.36 (t, J=9.2 Hz, 1H), 5.56 (d, J=4.3 Hz, 1H), 5.46 (d, J=4.8 Hz, 2H), 3.83 (d, J=6.6 Hz, 0.5H), 3.68 (d, J=6.6 Hz, 0.5H), 3.43 (s, 4H), 3.34 (s, 2H), 3.15 (d, J=11.5 Hz, 0.5H), 3.01 (d, J=10.9 Hz, 0.5H), 2.91 (m, 0.5H), 2.72 (m, 0.5H), 2,48 (d, J=12.9 Hz, 1H), 2.04 (m, 1H), 1.88 (m, 1H), 1.69 (m, 1H), 1.59 (m, 1H), 1.40 (dd, J=11.4, 6.5 Hz, 3H), 1.03 (t, .=7.3 Hz, 1.5H), 0.95 (t, J=7.3 Hz, 1.5H), 0.75 (t, J=7.3 Hz, 1.5H), 0.65 (t, J=7.5 Hz, 1.5H) ppm. MS: M/e 491 (M+1)⁺.

### Compound A77: 2-(7-((2S,5R)-2,5-diethyl-4-(1-(pyrazolo[1,5-a]pyridin-6-yl)ethyl)piperazin-1-yl)-4-methyl-5-oxo-4,5-dihydro-2H-pyrazolo[4,3-b]pyridin-2-yl)acetonitrile

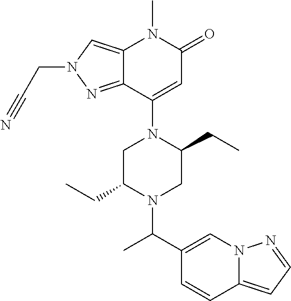 [SMILES: CC[C@H]1CN(C(C)c2ccc3ccnn3c2)[C@H](CC)CN1c1cc(=O)n(C)c2cn(CC#N)nc12]

### Step A: 1-(pyrazolo[1,5-a]pyridin-6-yl)ethan-1-one

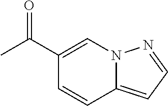

To a solution of 6-bromopyrazolo[1,5-a]pyridine (600 mg, 3 mol) in toluene (15 mL) was added Pd(PPh₃)₂Cl₂ (210 mg, 0.3 mol) and tributyl(1-ethoxyvinyl)stannane (1.6 g, 4.5 mmol). The reaction mixture was protected by N₂ atmosphere and stirred at 90° C. for 16 hours. The mixture was added 4N HCl in dioxane (0.5 ml) and stirred at RT for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (155 mg, 32.2%). MS: M/e 161 (M+1)”.

### Step E: 1-(pyrazolo[1.5-a]pyridin-6-yl)ethan-1-ol

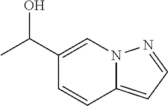

To a solution of 1-(pyrazolo[1,5-a]pyridin-6-yl)ethan-1-one (155 mg, 0.95 mol) in MeOH was added NaBH₄ (38 mg, 1 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (100 mg, 64.5%). MS: M/e 163 (M+1)⁺.

### Step F: 2-(7-((2S,5R)-2,5-diethyl-4-(1-(pyrazolo[1,5-a]pyridin-6-yl)ethyl)piperazin-1-yl)-4-methyl-5-oxo-4,5-dihydro-2H-pyrazolo[4,3-b]pyridin-2-yl)acetonitrile

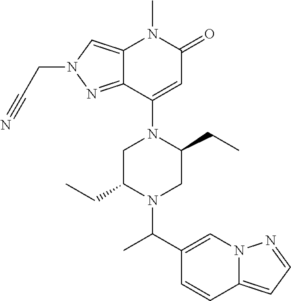 [SMILES: CC[C@H]1CN(C(C)c2ccc3ccnn3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(pyrazolo[1,5-a]pyridin-6-yl)ethan-1-ol (60 mg, 0.37 mmol) and (cyanomethyl)trimethylphosphonium iodide (241 mg, 1 mmol) in CH₃CN (4 mL) was added DIPEA (487 mg, 3 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method A) to give the Compound A77a (2 mg) and Compound A77b (3 mg).

Compound A77a (the earlier peak): ¹H NMR (500 MHz, CD₃OD) δ 8.47 (s, 1H), 7.92 (d, J=1.9 Hz, 2H), 7.66 (d, J=9.2 Hz, 1H), 7.35 (d, .=9.2 Hz, 1H), 6.58 (d, J=2.1 Hz, 1H), 5.57 (s, 1H), 5.46 (s, 2H), 3.84 (q, J=6.4 Hz, 1H), 3.43 (s, 3H), 3.34 (s, 1H), 3.32 (s, 2H), 3.02 (d, 0.1=11.6 Hz, 1H), 2.91 (dd, J=12.0, 3.7 Hz, 1H), 2,52 (s, 1H), 2.10 (m, 1H), 1.83 (m, 1H), 1.59 (d, J=7.1 Hz, 2H), 1.42 (d, J=6.5 Hz, 3H), 0.95 (t, J=7.4 Hz, 3H), 0.74 (t, J=7.4 Hz, 3H) ppm. MS: M/e 473 (M+1)⁺.

Compound A77b (the later peak): ¹H NMR (400 MHz, CD₃OD) δ 8.49 (s, 1H), 7.92 (m, 2H), 7.63 (d, J=9.1 Hz, 1H), 7.37 (d, J=9.2 Hz, 1H), 6.57 (s, 1H), 5.56 (s, 1H), 5.47 (s, 2H), 3.69 (q, J=6.5 Hz, 1H), 3.52 (d, J=13.8 Hz, 1H), 3.43 (s, 3H), 3.32 (s, 2H), 3.16 (d, J=8.4 Hz, 1H), 2.72 (dd, J=12.6, 3.7 Hz, 1H), 2,49 (d, J=12.2 Hz, 1H), 1.94 (m, 1H), 1.69 (m, 2H), 1.57 (m, 1H), 1.40 (d, J=6.6 Hz, 3H), 1.03 (t, J=7.3 Hz, 3H), 0.64 (t, J=7.4 Hz, 3H) ppm. MS: M/e 473 (M+1)⁺.

### Compound A78: 2-(7-((2S,5R)-2,5-diethyl-4-(1-(3-methyl-3H-imidazo[4,5-b]pyridin-6-yl)ethyl)piperazin-1-yl)-4-methyl-5-oxo-4,5-dihydro-2H-pyrazolo[4,3-b]pyridin-2-yl)acetonitrile

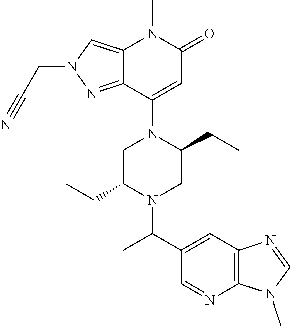 [SMILES: CC[C@H]1CN(C(C)c2cnc3c(c2)ncn3C)[C@H](CC)CN1c1cc(=O)n(C)c2cn(CC#N)nc12]

### Step A: 6-bromo-3-methyl-3H-imidazo[4.5-b]pyridine

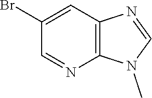

To a solution of 6-bromo-3H-imidazo[4,5-b]pyridine (0.396 g, 2 mol) in DMSO (15 mL) was added NaHMDS (2M, 1.5 ml, 3 mol) and CH₃I (858 mg, 6 mmol). The reaction mixture was protected by N₂ atmosphere and stirred at RT for 2 hrs. The mixture was added H₂O (20 ml) and extracted with EA (20 mL×3), then concentrated by using a rotary evaporator to give a residue. The resulting residue was purified by flash column chromatography to give the titled compound (330 mg, 78.2%). MS: M/e 212 (M+1)⁺.

### Step B: 1-(3-methyl-3H-imidazo[4.5-b]pyridin-6-yl)ethan-1-one

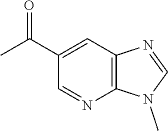

To a solution of 6-bromo-3-methyl-3H-imidazo[4,5-b]pyridine (330 mg, 1.57 mol) in toluene (10 mL) was added Pd(PPh₃)₂Cl₂ (70 mg, 0.1 mol) and tributyl(1-ethoxyvinyl)stannane (860 mg, 2.3 mmol). The reaction mixture was protected by N₂ atmosphere and stirred at 90° C. for 16 hrs. The mixture was added 4N HCl in dioxane (0.5 ml) and stirred at RT for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (220 mg, 80.3%). MS: We 176 (M+1)⁺.

### Step C: 1-(3-methyl-3H-imidazo[4.5-b]pyridin-6-yl)ethan-1-ol

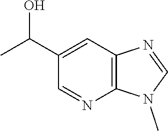

To a solution of 1-(3-methyl-3H-imidazo[4,5-b]pyridin-6-yl)ethan-1-one (220 mg, 1,25 mol) in MeOH was added NaBH₄ (47 mg, 1,25 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (30 mg, 13.6%). MS: M/e 178 (M+1)⁺.

### Step D: 2-(7-((2S,5R)-2,5-diethyl-4-(1-(3-methyl-3H-imidazo[4.5-b]pyridin-6-yl)ethyl)piperazin-1-yl)-4-methyl-5-oxo-4,5-dihydro-2H-pyrazolo[4,3-b]pyridin-2-yl)acetonitrile

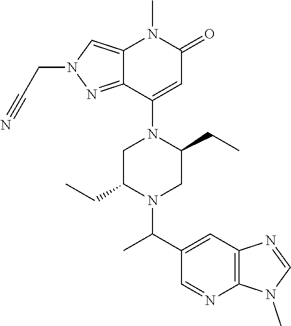 [SMILES: CC[C@H]1CN(C(C)c2cnc3c(c2)ncn3C)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(3-methyl-3H-imidazo[4,5-b]pyridin-6-yl)ethan-1-ol (30 mg, 0.17 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method A) to give the titled compound (4.6 mg, 9%). ¹H NMR (400 MHz, CD₃OD) δ 8.45 (dd, J=12,4, 1.5 Hz, 1H), 8.35 (d, J=7.8 Hz, 1H), 8.11 (dd, J=19.4, 1.6 Hz, 1H), 7.92 (d, J=3.9 Hz, 1H), 5.56 (s, 1H), 5.46 (d, J=6.1 Hz, 2H), 4.00 (d, J=6.5 Hz, 0.5H), 3.94 (d, J=4.1 Hz, 3H), 3.85 (d, J=6.3 Hz, 0.5H), 3.54 (d, J=12.7 Hz, 0.5H), 3.43 (s, 3H), 3.28 (m, 1.5H), 3.22 (d, J=10.6 Hz, 0.5H), 3.07 (d, J=10.4 Hz, 0.5H), 2.93 (dd, J=12.0, 3.8 Hz, 0.5H), 2.71 (d, J=8.7 Hz, 0.5H), 2.35 (d, J=13.2 Hz, 1H), 2.15-2.02 (m, 1H), 1.87 (m, 1H), 1.69 (m, 1H), 1.59 (m, 2H), 1.44 (m, 3H), 1.05 (t, J=7.3 Hz, 1.5H), 0.97 (t, J=7.4 Hz, 1.5H), 0.69 (t, J=7.4 Hz, 1.5H), 0.55 (t, J=7.4 Hz, 1.5H) ppm. MS: M/e 488 (M+1)⁺.

### Compound A79: 2-(7-((2S,5R)-2,5-diethyl-4-(1-(3-methylimidazo[1,2-a]pyridin-7-yl)ethyl)piperazin-1-yl)-4-methyl-5-oxo-4,5-dihydro-2H-pyrazolo[4,3-b]pyridin-2-yl)acetonitrile

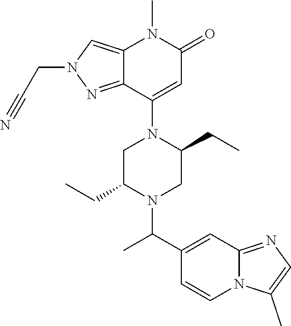 [SMILES: CC[C@H]1CN(C(C)c2ccn3c(C)cnc3c2)[C@H](CC)CN1c1cc(=O)n(C)c2cn(CC#N)nc12]

### Step A: tert-butyl (4-bromopyridin-2-yl)carbamate

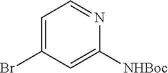 [SMILES: CC(C)(C)OC(=O)Nc1cc(Br)ccn1]

A solution of 4-bromopyridin-2-amine (1.73 g, 10 mol) in DCM (25 mL) was added di-tert-butyl decarbonate (2,56 g, 12 mmol), DMAP (122 mg, 1 mmol) and Et₃N (2.02 g, 20 mmol). The reaction mixture was stirred at RT for 3 hrs. The mixture was concentrated in vacuo. The resulting residue was added H₂O (20 ml) and extracted with EA (20 mL×3), then concentrated by using a rotary evaporator to give a residue. The resulting residue was purified by flash column chromatography to give the titled compound (1 g, 36.7%). MS: M/e 273 (M+1)⁺.

### Step B: tert-butyl (4-bromopyridin-2-yl)(prop-2-yn-1-yl)carbamate

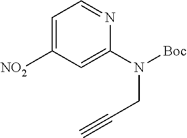 [SMILES: C#CCN(C(=O)OC(C)(C)C)c1cc([N+](=O)[O-])ccn1]

To a solution of tert-butyl (4-bromopyridin-2-yl)carbamate (1 g, 3.6 mol) in DMF (10 mL) was added NaH (60% in oil, 288 mg, 7.6 mol) and bromoethyne (654 mg, 5.5 mmol). The reaction mixture was stirred at RT overnight. The mixture was slowly added H₂O (20 ml) at 0° C. and extracted with EA (20 mL×3). The organic phase was dried with Na₂SO₄ and concentrated by using a rotary evaporator to give a residue. The resulting residue was purified by flash column chromatography to give the titled compound (0.85 g, 76.5%). MS: M/e 311 (M+1)⁺.

C: 7-bromo-3-methylimidazo[1,2-a]pyridine

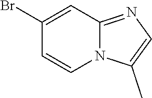

To a solution of tert-butyl (4-bromopyridin-2-yl)(prop-2-yn-1-yl)carbamate (424 mg, 2 mol) in THF (10 mL) was added t-BuOK (268 mg, 2,4 mmol). The reaction mixture was stirred at RT for 40 mins. The mixture was added H₂O (20 ml) and extracted with EA (20 mL×3), then concentrated by using a rotary evaporator to give a residue. The resulting residue was purified by flash column chromatography to give the titled compound (150 mg, 35.7%). MS: M/e 211 (M+1)⁺.

### Step D: 1-(3-methylimidazo[1,2-a]pyridin-7-yl)ethan-1-one

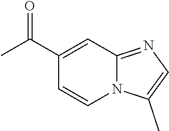

To a solution of 7-bromo-3-methylimidazo[1,2-a]pyridine (150 mg, 0.7 mol) in toluene (10 mL) was added Pd(PPh₃)₂Cl₂ (70 mg, 0.1 mol) and tributyl(1-ethoxyvinyl)stannane (365 mg, 1 mmol). The reaction mixture was protected by N₂ atmosphere and stirred at 90° C. for 16 hrs. The mixture was added 4N HCl in dioxane (0.5 ml) and stirred at RT for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (30 mg, 24.7%). MS: We 175 (M+1)⁺.

### Step E: 1-(3-methylimidazo[1,2-a]pyridin-7-yl)ethan-1-ol

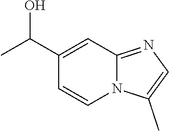

To a solution of 1-(3-methylimidazo[1,2-a]pyridin-7-yl)ethan-1-one (30 mg, 0.17 mol) in MeOH was added NaBH₄ (19 mg, 0.9 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (20 mg, 66.7%). MS: M/e 177 (M+1)⁺.

### Step F: 2-(7-((2S,5R)-2,5-diethyl-4-(1-(3-methylimidazo[1,2-a]pyridin-7-yl)ethyl)piperazin-1-yl)-4-methyl-5-oxo-4,5-dihydro-2H-pyrazolo[4,3-b]pyridin-2-yl)acetonitrile

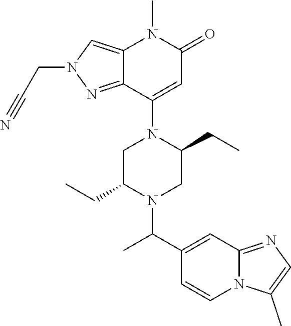 [SMILES: CC[C@H]1CN(C(C)c2ccn3c(C)cnc3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(3-methylimidazo[1,2-a]pyridin-7-yl)ethan-1-ol (20 mg, 0.11 mmol) and (cyanomethyl)trimethylphosphonium iodide (120 mg, 0.5 mmol) in CH₃CN (10 mL) was added DIPEA (129 mg, 1 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method A) to give the Compound A79a (0.35 mg, as a formic acid salt) and Compound A79b (0.3 mg).

Compound A79a (the earlier peak): ¹H NMR (500 MHz, CD₃OD) δ 8.48 (s, 1H), 8.21 (d, J=7.1 Hz, 1H), 7.92 (s, 1H), 7.48 (s, 1H), 7.34 (s, 1H), 7.16 (d, J=7.8 Hz, 1H), 5.57 (s, 1H), 5.45 (s, 2H), 3.88 (d, J=6.3 Hz, 1H), 3.44 (d, J=5.4 Hz, 4H), 3.16 (s, 1H), 3.03 (d, J=12.1 Hz, 1H), 2.91 (d, J=8.8 Hz, 1H), 2,52 (s, 3H), 2,48 (m, 1H), 2.13 (dd, J=14.2, 6.9 Hz, 1H), 2.03 (d, J=4.1 Hz, 1H), 1.86 (dd, J=14.1, 6.8 Hz, 1H), 1.59 (d, J=7.4 Hz, 2H), 1.40 (d, J=6.4 Hz, 3H), 0.96 (t, J=7.4 Hz, 3H), 0.73 (t, J=7.3 Hz, 3H) ppm. MS: M/e 487 (M+1)⁺.

Compound A79b (the later peak): ¹H NMR (400 MHz, CD₃OD) δ 8.28 (s, 1H), 7.92 (s, 1H), 7.76 (s, 1H), 7.42 (s, 1H), 7.26 (s, 1H), 5.56 (s, 1H), 5.47 (s, 2H), 3.74 (d, J=6.9 Hz, 1H), 3.53 (d, J=14.6 Hz, 1H), 3.43 (s, 4H), 3.37 (s, 1H), 3.16 (s, 1H), 2.73 (d, J=9.6 Hz, 1H), 2,53 (s, 3H), 2,40 (d, J=11.9 Hz, 1H), 1.93 (s, 1H), 1.74 (s, 1H), 1.60 (s, 2H), 1.02 (t, J=7.2 Hz, 3H), 0.90 (t, J=6.8 Hz, 3H), 0.64 (d, J=6.8 Hz, 3H) ppm. MS: M/e 487 (M+1)⁺.

### Compound A80: 2-(7-((2S,5R)-2,5-diethyl-4-(1-(imidazo[1,2-a]pyridin-7-yl)ethyl)piperazin-1-yl)-4-methyl-5-oxo-4,5-dihydro-2H-pyrazolo[4,3-b]pyridin-2-yl)acetonitrile

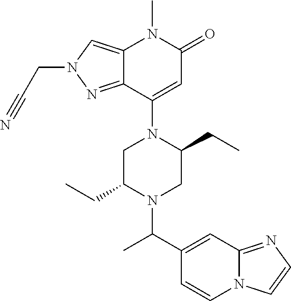 [SMILES: CC[C@H]1CN(C(C)c2ccn3ccnc3c2)[C@H](CC)CN1c1cc(=O)n(C)c2cn(CC#N)nc12]

### Step A: 1-(imidazo[1,2-a]pyridin-7-yl)ethan-1-ol

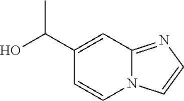

To a solution of imidazo[1,2-a]pyridine-7-carbaldehyde (120 mg, 0.82 mmol) in THF (6 ml) under N₂, was added MeMgBr (3M, 0.315 ml, 0.94 mmol) at 0° C. and stirred for 20 min at 0° C. The solution was quenched with water (10 ml) and then extracted with EA (10 ml×2). The organic layer was dried over Na₂SO₄ and concentrated to dryness. The resulting residue was purified by flash column chromatography with 0-10% MeOH in DCM to give the titled compound (100 mg, 75%). MS: M/e 163 (M+1)⁺.

### Step B: 2-(7-((2S,5R)-2,5-diethyl-4-(1-(imidazo[1,2-a]pyridin-7-yl)ethyl)piperazin-1-yl)-4-methyl-5-oxo-4,5-dihydro-2H-pyrazolo[4,3-b]pyridin-2-yl)acetonitrile

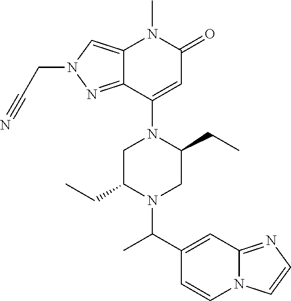 [SMILES: CC[C@H]1CN(C(C)c2ccn3ccnc3c2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol), 1-(imidazo[1,2-a]pyridin-7-yl)ethan-1-ol (74 mg, 0.46 mmol), (cyanomethyl)trimethylphosphonium iodide (111 mg, 0.46 mmol) and DIPEA (197 mg, 1.53 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A80, which was further separated into Compound A80a (2 mg) and Compound A80b (3 mg) by Prep-HPLC (Method A).

Compound A80a (the earlier peak): ¹H NMR (400 MHz, DMSO-d₆) δ 8.50 (d, J=6.8 Hz, 1H), 8.40 (s, 0.39H), 7.98 (s, 1H), 7.89 (s, 1H), 7.50 (d, J=21.8 Hz, 2H), 6.95 (d, J=7.3 Hz, 1H), 5.61 (s, 2H), 5.40 (s, 1H), 3.80 (q, J=6.2 Hz, 1H), 3.37 (s, 2H), 3.27 (s, 3H), 3.19-3.12 (m, 1H), 2.91 (d, J=11.9 Hz, 1H), 2.80 (d, J=11.9 Hz, 1H), 2.38 (d, J=8.9 Hz, 1H), 2.08-1.97 (m, 1H), 1.71-1.58 (m, 1H), 1.56-1.42 (m, 2H), 1.31 (d, J=6.3 Hz, 3H), 0.86 (t, J=7.1 Hz, 3H), 0.65 (t, J=7.1 Hz, 3H) ppm. MS: M/e 473 (M+1)⁺.

Compound A80b (the later peak): ¹H NMR (400 MHz, DMSO-d⁶) 8.50 (d, J=7.0 Hz, 1H), 8.32 (s, 0.80H), 7.99 (s, 1H), 7.89 (s, 1H), 7.50 (d, J=19.7 Hz, 2H), 6.96 (d, J=6.5 Hz, 1H), 5.62 (s, 2H), 5.40 (s, 1H), 3.65 (q, J=6.3 Hz, 1H), 3.48 (s, 1H), 3.39 (d, J=12.8 Hz, 2H), 3.27 (s, 3H), 3.04 (d, J=9.4 Hz, 1H), 2,59 (d, J=8.9 Hz, 1H), 2.32 (d, J=11.7 Hz, 1H), 1.91-1.79 (m, 1H), 1.65-1.38 (m, 3H), 1,27 (d, J=6.2 Hz, 3H), 0.95 (t, J=7.1 Hz, 3H), 0.58 (t, J=7.3 Hz, 3H) ppm. MS: M/e 473 (M+1)⁺.

### Compound A81: 2-(7-((2S,5R)-4-(1-([1,2,4]triazolo[1,5-a]pyridin-6-yl)ethyl)-2,5-diethylpiperazin-1-y )₄-methyl-5-oxo-4,5-dihydro-2H-pyrazolo[4,3-b]pyridin-2-yl)acetonitrile

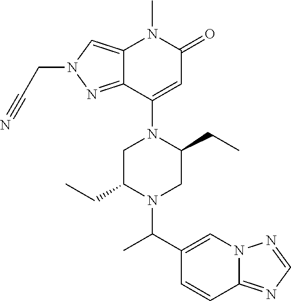 [SMILES: CC[C@H]1CN(C(C)c2ccc3ncnn3c2)[C@H](CC)CN1c1cc(=O)n(C)c2cn(CC#N)nc12]

### Step A: 1-([1,2,4]triazolo[1,5-a]pyridin-6-yl)ethan-1-one

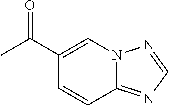

A mixture of 6-bromo-[1,2,4]triazolo[1,5-a]pyridine (1 g, 5.07 mmol), tributyl(1-ethoxyvinyl)stannane (2.20 g, 6.09 mmol) and Pd(PPh₃)₂Cl₂ (356 mg, 0.51 mmol) in toluene (10 mL) was stirred at 100° C. under N₂ overnight. The solution was added HCl (0.3 mL, 4 M in 1,4-dioxane) and the mixture was stirred at room temperature for 0.5 hour. The reaction mixture was diluted with EA and concentrated to dryness. The resulting residue was purified by flash column chromatography to give the titled compound (620 mg, 76%). MS: M/e 162 (M+1)⁺.

### Step B: 1-([1,2,4]triazolo[1,5-a]pyridin-6-yl)ethan-1-ol

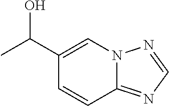

To a solution of 1-([1,2,4]triazolo[1,5-a]pyridin-6-yl)ethan-1-one (500 mg, 3.11 mmol) in MeOH (15 mL) was added NaBH₄ (177 mg, 4.66 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (460 mg, 91%). MS: M/e 164 (M+1)⁺.

### Step C: 2-(7-((2S,5R)-4-(1-([1,2,4]triazolo[1,5-a]pyridin-6-yl)ethyl)-2,5-diethylpiperazin-1-yl)-4-methyl-5-oxo-4,5-dihydro-2H-pyrazolo[4,3-b]pyridin-2-yl)acetonitrile

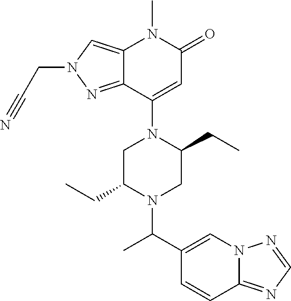 [SMILES: CC[C@H]1CN(C(C)c2ccc3ncnn3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-([1,2,4]triazolo[1,5-a]pyridin-6-yl)ethan-1-ol (25 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled Compound A81a (10 mg) and Compound A81b (12 mg).

Compound A81a (the earlier peak) ¹H NMR (400 MHz, CD₃OD) δ 8.78 (s, 1H), 8.40 (s, 1H), 7.92 (s, 1H), 7.86 (d, J=9.2 Hz, 1H), 7.79 (d, J=9.1 Hz, 1H), 5.57 (s, 1H), 5.46 (s, 2H), 3.98 (d, J=6.6 Hz, 1H), 3.43 (s, 3H), 3.34 (s, 2H), 3.17-3.00 (m, 2H), 2.97-2,43 (m, 2H), 2.15-1.69 (m, 2H), 1.60 (d, J=8.0 Hz, 2H), 1.45 (d, J=6.5 Hz, 3H), 0.95 (t, J=7.3 Hz, 3H), 0.76 (t, J=7.3 Hz, 3H). MS: M/e 474 (M+1)⁺.

Compound A81b (the later peak): ¹H NMR (400 MHz, CD₃OD) δ 8.78 (s, 1H), 8.39 (s, 1H), 7.93 (s, 1H), 7.88 (d, J=9.9 Hz, 1H), 7.77 (d, J=9.2 Hz, 1H), 5.56 (s, 11H), 5.47 (s, 2H), 3.85-3.79 (m, 1H), 3.54 (d, J=12.7 Hz, 1H), 3.43 (s, 3H), 3.29-3.14 (m, 3H), 2.75 (d, J=9.1 Hz, 2H), 2,45-1.80 (m, 1H), 1.79-1.65 (m, 2H), 1.61-1.52 (m, 1H), 1.42 (d, J=6.5 Hz, 3H), 1.04 (t, J=7.4 Hz, 3H), 0.63 (t, J=7.4 Hz, 3H). MS: M/e 474 (M+1)⁺.

### Compound A82: 2-(7-((2S,5R)-2,5-diethyl-4-(1-(imidazo[1,2-a]pyridin-6-yl)ethyl)piperazin-1-yl)-4-methyl-5-oxo-4,5-dihydro-2H-pyrazolo[4,3-b]pyridin-2-yl)acetonitrile

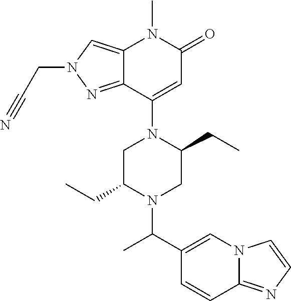 [SMILES: CC[C@H]1CN(C(C)c2ccc3nccn3c2)[C@H](CC)CN1c1cc(=O)n(C)c2cn(CC#N)nc12]

### Step A: 1-(imidazole[1,2-a]pyridin-6-yl)ethan-1-one

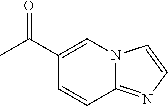

A mixture of 6-bromoimidazo[1,2-a]pyridine (1 g, 5.10 mmol), tributyl(1-ethoxyvinyl)stannane (2.21 g, 6.12 mmol) and Pd(PPh₃)₂Cl₂ (358 mg, 0.51 mmol) in toluene (10 mL) was stirred at 100° C. under N₂ overnight. The solution was added HCl (0.3 mL, 4 M in 1,4-dioxane) and the mixture was stirred at room temperature for 0.5 hour. The reaction mixture was diluted with EA and concentrated to dryness. The resulting residue was purified by flash column chromatography to give the titled compound (660 mg, 81%). MS: M/e 161 (M+1)⁺.

### Step B: 1-(imidazo[1,2-a]pyridin-6-yl)ethan-1-ol

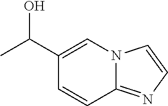

To a solution of 1-(imidazo[1,2-a]pyridin-6-yl)ethan-1-one (500 mg, 3.12 mmol) in MeOH (15 mL) was added NaBH₄ (178 mg, 4.69 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (400 mg, 79%). MS: M/e 163 (M+1)⁺.

### Step C: 2-(7-((2S,5R)-2,5-diethyl-4-(1-(imidazo[1,2-a]pyridin-6-yl)ethyl)piperazin-1-yl)-4-methyl-5-oxo-4,5-dihydro-2H-pyrazolo[4.-b]pyridin-2-yl)acetonitrile

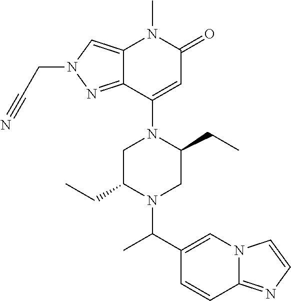 [SMILES: CC[C@H]1CN(C(C)c2ccc3nccn3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(imidazo[1,2-a]pyridin-6-yl)ethan-1-ol (25 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled Compound A82a (14 mg) and Compound A82b (14 mg, 39%).

Compound A82a (the earlier peak) ¹H NMR (400 MHz, CD₃OD) δ 8.52 (s, 1H), 7.92 (s, 2H), 7.75 (s, 1H), 7.66 (s, 1H), 7.62 (s, 1H), 5.57 (s, 1H), 5.46 (s, 2H), 3.88 (s, 1H), 3.43 (s, 3H), 3.13 (s, 1H), 3.02 (d, J=11.8 Hz, 1H), 2.94 (s, 1H), 2,49 (s, 1H), 2.09 (s, 1H), 1.83 (s, 1H), 1.59 (s, 2H), 1.43 (d, J=5.4 Hz, 3H), 1,29 (s, 2H), 0.95 (s, 3H), 0.75 (s, 3H). MS: M/e 473 (M+1)⁺.

Compound A82b (the later peak): ¹H NMR (400 MHz, CD₃OD) δ 8.54 (s, 1H), 7.93 (s, 2H), 7.77 (s, 1H), 7.71 (s, 2H), 5.56 (s, 1H), 5.47 (s, 2H), 3.74 (s, 1H), 3.53 (d, J=12.3 Hz, 1H), 3.43 (s, 3H), 3.28-3.11 (m, 3H), 2.77-2.69 (m, 1H), 2,43 (d, J=11.2 Hz, 1H), 1.96-1.87 (m, 1H), 1.75-1.65 (m, 2H), 1.60-1.53 (m, 1H), 1.41 (d, J=4.3 Hz, 3H), 1.03 (s, 3H), 0.63 (s, 3H). MS: M/e473 (M+1)⁺.

### Compound A83: 2-(7-((2S,5R)-2,5-diethyl-4-(1-(imidazo[1,5-a]pyridin-6-yl)ethyl)piperazin-1-yl)-4-methyl-5-oxo-4,5-dihydro-2H-pyrazolo[4,3-b]pyridin-2-yl)acetonitrile

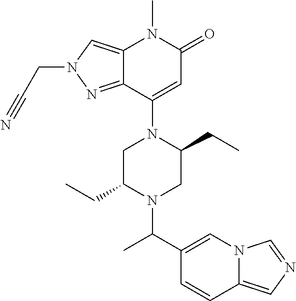 [SMILES: CC[C@H]1CN(C(C)c2ccc3cncn3c2)[C@H](CC)CN1c1cc(=O)n(C)c2cn(CC#N)nc12]

### Step A: 1-(imidazo[1.5-a]pyridin-6-yl)ethan-1-one

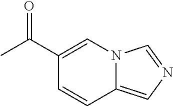

A mixture of 6-bromoimidazo[1,5-a]pyridine (1 g, 5.10 mmol), tributyl(1-ethoxyvinyl)stannane (2.21 g, 6.12 mmol) and Pd(PPh₃)₂Cl₂ (358 mg, 0.51 mmol) in toluene (10 mL) was stirred at 100° C. under N₂ overnight. The solution was added HCl (0.3 mL, 4 M in 1,4-dioxane) and the mixture was stirred at room temperature for 0.5 hour. The reaction mixture was diluted with EA and concentrated to dryness. The resulting residue was purified by flash column chromatography to give the titled compound (580 mg, 71%). MS: M/e 161 (M+1)⁺.

### Step B: 14imidazo[1,5-a]pyridin-6-yl)ethan-1-ol

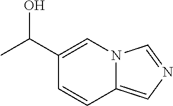

To a solution of 1-(imidazo[1,5-a]pyridin-6-yl)ethan-1-one (500 mg, 3.12 mmol) in MeOH (15 mL) was added NaBH₄ (178 mg, 4.69 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (360 mg, 71%). MS: M/e 163 (M+1)⁺.

### Step C: 2-(7-((2S,5R)-2,5-diethyl-4-(1-(imidazo[1,5-a]pyridin-6-yl)ethyl)piperazin-1-yl)-4-methyl-5-oxo-4,5-dihydro-2H-pyrazolo[4,3-b]pyridin-2-yl)acetonitrile

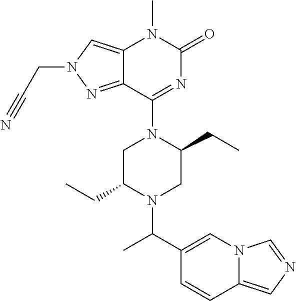 [SMILES: CC[C@H]1CN(C(C)c2ccc3cncn3c2)[C@H](CC)CN1c1nc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(imidazo[1,5-a]pyridin-6-yl)ethan-1-ol (25 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled Compound A83a (18 mg) and Compound A83b (14 mg).

Compound A83a (the earlier peak): ¹H NMR (400 MHz, CD₃OD) δ 8.27 (s, 1H), 8.19 (s, 1H), 7.92 (s, 1H), 7.53 (d, J=9.1 Hz, 1H), 7.33 (s, 1H), 6.97 (d, J=9.5 Hz, 1H), 5.57 (s, 1H), 5.46 (s, 2H), 3.79-3.44 (m, 3H), 3.43 (s, 3H), 3.24-3.10 (m, 1H), 3.00 (d, J=11.9 Hz, 1H), 2.88 (d, J=9.3 Hz, 1H), 2,56 (s, 1H), 2.08 (s, 1H), 1.82 (s, 1H), 1.58 (s, 2H), 1.39 (d, J=6.5 Hz, 3H), 0.95 (t, J=7.4 Hz, 3H), 0.76 (t, J=7.2 Hz, 3H). MS: M/e 473 (M+1)⁺.

Compound A83b (the later peak): ¹H NMR (400 MHz, CD₃OD) δ 8.25 (s, 1H), 8.18 (s, 1H), 7.93 (s, 1H), 7.51 (d, J=9.5 Hz, 1H), 7.32 (s, 1H), 6.99 (d, J=9.5 Hz, 1H), 5.56 (s, 1H), 5.47 (s, 2H), 3.58 (d, J=6.3 Hz, 1H), 3.50 (d, J=14.6 Hz, 2H), 3.43 (s, 3H), 3.26-3.10 (m, 2H), 2.74-2.68 (m, J=8.5 Hz, 1H), 2,52 (d, J=11.7 Hz, 1H), 2.00-1.92 (m, 1H), 1.72-1.63 (m, 2H), 1.59-1.52 (m, 1H), 1.38 (d, J=6.5 Hz, 3H), 1.03 (t, J=7.3 Hz, 3H), 0.67 (t, J=7.4 Hz, 3H). MS: M/e 473 (M+1)⁺.

### Compound A84: 2-(7-((2S,5R)-4-(1-(benzo[d]oxazol-6-yl)ethyl)-2,5-diethylpiperazin-1-yl)-4-methyl-5-oxo-4,5-dihydro-2H-pyrazolo[4,3-b]pyridin-2-yl)acetonitrile

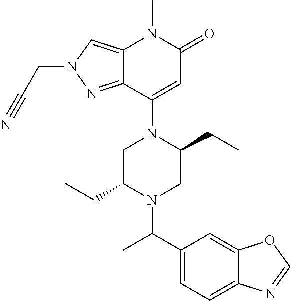 [SMILES: CC[C@H]1CN(C(C)c2ccc3ncoc3c2)[C@H](CC)CN1c1cc(=O)n(C)c2cn(CC#N)nc12]

### Step A: 1-(benzo[d]oxazol-6-yl)ethan-1-one

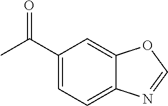

To a solution of 6-bromobenzo[d]oxazole (1 g, 5 mol) in toluene (15 mL) was added Pd(PPh₃)₂Cl₂ (140 mg, 0.2 mol) and tributyl(1-ethoxyvinyl)stannane (2.7 g, 7.5 mmol). The reaction mixture was protected by N₂ atmosphere and stirred at 90° C. for 16 hrs. The mixture was added 4N HCl in dioxane (0.5 ml) and stirred at RT for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (500 mg, 62.1%). MS: M/e 162 (M+1)⁺.

### Step B: 1-(benzo[d]oxazol-6-yl)ethan-1-ol

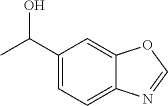

To a solution of 1-(benzo[d]oxazol-6-yl)ethan-1-one (500 mg, 3.1 mol) in MeOH was added NaBH₄ (118 mg, 3 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (300 mg, 60%). MS: M/e 164 (M+1)⁺.

### Step C: 2-(7-((2S,5R-4-(1-(benzo[d]oxazol-6-yl)ethyl)-2,5-diethylpiperazin-1-yl)-4-methyl-5-oxo-4,5-dihydro-2H-pyrazolo[4,3-b]pyridin-2-yl)acetonitrile

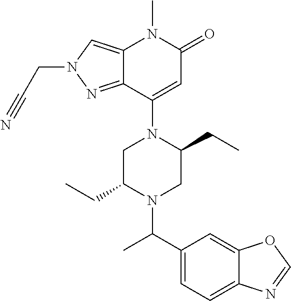 [SMILES: CC[C@H]1CN(C(C)c2ccc3ncoc3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(benzo[d]oxazol-6-yl)ethan-1-ol (50 mg, 0.3 mmol) and (cyanomethyl)trimethylphosphonium iodide (120 mg, 0.5 mmol) in CH₃CN (10 mL) was added DIPEA (129 mg, 1 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method B) to give the titled Compound A84a (3 mg) and Compound A84b (3 mg).

Compound A84a (the earlier peak): ¹H NMR (500 MHz, CD₃OD) δ 8.45 (s, 1H), 7.92 (s, 1H), 7.72 (d, J=8.6 Hz, 2H), 7.48 (d, J=8.2 Hz, 1H), 5.56 (s, 1H), 5.45 (s, 2H), 3.92 (d, 0.1 =6.5 Hz, 1H), 3.43 (s, 3H), 3.28 (m, 3H), 3.05 (d, J=11.3 Hz, 1H), 2.90 (dd, J=11.9, 3.8 Hz, 1H), 2,41 (s, 1H), 2.15 (m, 1H), 1.85 (m, 1H), 1.55 (m, 2H), 1.40 (d, 0.1=6.4 Hz, 3H), 0.97 (t, J=7.4 Hz, 3H), 0.68 (t, J=7.3 Hz, 3H) ppm. MS: M/e 474 (M+1)⁺.

Compound A84b (the later peak): ¹H NMR (400 MHz, CD₃OD) δ 8.44 (s, 1H), 7.92 (s, 1H), 7.74 (s, 1H), 7.69 (d, J=8.2 Hz, 1H), 7.50 (d, J=8.2 Hz, 1H), 5.55 (s, 1H), 5.47 (s, 2H), 3.77 (d, J=6.5 Hz, 1H), 3.53 (d, J=12,5 Hz, 1H), 3.43 (s, 3H), 3.32 (s, 1H), 3.19 (d, J=10.0 Hz, 1H), 2.68 (m, 2H), 2.36 (d, J=12.1 Hz, 1H), 1.98 (m, 1H), 1.64 (m, 3H), 1.37 (d, J=6.5 Hz, 3H), 1.04 (t, J=7.3 Hz, 3H), 0.58 (t, J=7.4 Hz, 3H) ppm. MS: M/e 474 (M+1)⁺.

### Compound A85: 2-(7-((2S,5R)-4-(1-(benzo[dioxazol-6-yl)ethyl)-5-ethyl-2-methylpiperazin-1-yl)-4-methyl-5-oxo-4,5-dihydro-2H-pyrazolo[4,3-b]pyridin-2-yl)acetonitrile

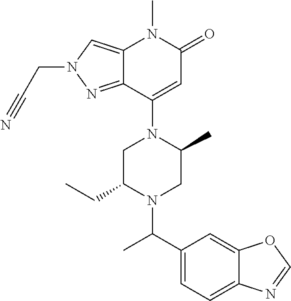 [SMILES: CC[C@@H]1CN(c2cc(=O)n(C)c3cn(CC#N)nc23)[C@@H](C)CN1C(C)c1ccc2ncoc2c1]

To a solution of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (192 mg, 0.6 mmol), 1-(benzo[d]oxazol-6-yl)ethan-1-ol (200 mg, 1,22 mmol) and (cyanomethyl)trimethylphosphonium iodide (433 mg, 1.8 mmol) in CH₃CN (10 mL) was added DIPEA (387 mg, 3 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method B) to give the titled Compound A85a (34 mg) and Compound A85b (44 mg).

Compound A85a (the earlier peak): ¹H NMR (400 MHz, CD₃OD) δ 8.44 (s, 1H), 7.90 (s, 1H), 7.70 (s, 2H), 7.47 (d, J=7.3 Hz, 1H), 5.56 (s, 1H), 5.43 (s, 2H), 3.91 (s, 1H), 3.42 (s, 3H), 3.30 (s, 2H), 3.24 (m, 1H), 2.97 (d, J=11.5 Hz, 1H), 2.86 (d, J=11.9 Hz, 1H), 2,42 (s, 1H), 1.56 (s, 2H), 1.38 (d, J=5.5 Hz, 6H), 0.71 (s, 3H) ppm. MS: M/e 460 (M+1)⁺.

Compound A85b (the later peak): ¹H NMR (400 MHz, CD₃OD) δ 8.42 (s, 1H), 7.91 (s, 1H), 7.73 (s, 1H), 7.66 (s, 1H), 7.50 (s, 1H), 5.56 (s, 1H), 5.45 (s, 2H), 3.77 (s, 1H), 3.55 (d, J=12.9 Hz, 1H), 3.44 (d, J=16.9 Hz, 3H), 3.24 (m, 2H), 3.19 (s, 1H), 2.75 (d, J=12,4 Hz, 1H), 2.23 (d, J=12.3 Hz, 1H), 1.70 (s, 1H), 1.57 (s, 1H), 1.36 (s, 3H), 1,20 (s, 3H), 1.05 (s, 3H) ppm. MS: We 460 (M+1)⁺.

### Compound A86 and Compound A87: 2-(7-((2S,5R)-2,5-diethyl-4-(1-(7-fluoro-2-methylbenzo[d]oxazol-6-yl)ethyl)piperazin-1-yl)-4-methyl-5-oxo-4,5-dihydro-2H-pyrazolo[4,3-b]pyridin-2-yl)acetonitrile and N-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-3-fluoro-2-hydroxyphenyl)acetamide

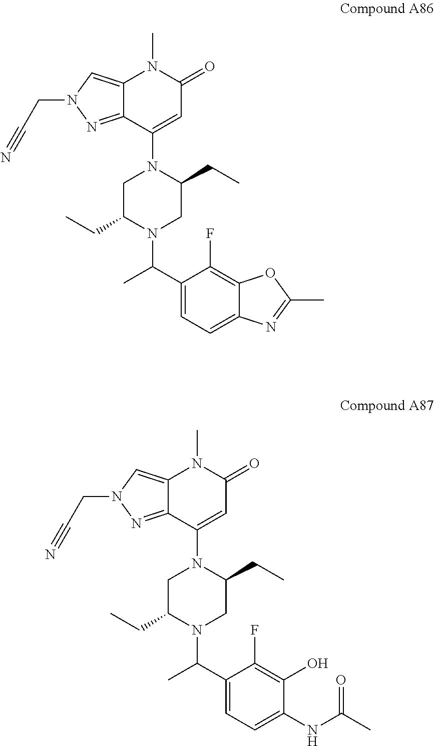 [SMILES: CC[C@H]1CN(C(C)c2ccc3nc(C)oc3c2F)[C@H](CC)CN1c1cc(=O)n(C)c2cn(CC#N)nc12 ; CC[C@H]1CN(C(C)c2ccc(NC(C)=O)c(O)c2F)[C@H](CC)CN1c1cc(=O)n(C)c2cn(CC#N)nc12]

### Step A: I-(7-fluoro-2-methylbenzo[d]oxazol-6-yl)ethan-1-one

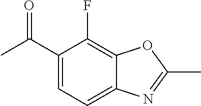

To a solution of 1-(4-amino-2-fluoro-3-hydroxyphenyl)ethan-1-one (500 g, 2.96 mmol) in 1,1,1-triethoxyethane (5 mL) was added 4-methylbenzenesulfonic acid (102 mg, 0.59 mmol). The resulting mixture was stirred at 140° C. overnight. The reaction mixture was diluted with DCM (200 mL). The organic layer was washed with water, dried over Na₂SO₄ and concentrated to give the titled compound (230 mg, 40%). MS: M/e 194 (M+1)⁺.

### Step B: 1-(7-fluoro-2-methylbenzooxazol-6-yl)ethan-1-ol

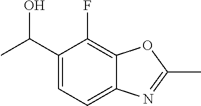

To a solution of 1-(7-fluoro-2-methylbenzo[d]oxazol-6-yl)ethan-1-one (230 mg, 1.19 mmol) in MeOH (15 mL) was added NaBH₄ (91 mg, 2.38 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (200 mg, 86%). MS: M/e 196 (M+1)⁺.

### Step C: 2-(7-((2S,5R)-2,5-diethyl-4-(1-(7-fluoro-2-methylbenzo[d]oxazol-6-yl)ethyl)piperazin-1-yl)-4-methyl-5-oxo-4,5-dihydro-2H-pyrazolo[4,3-b]pyridin-2-yl)acetonitrile

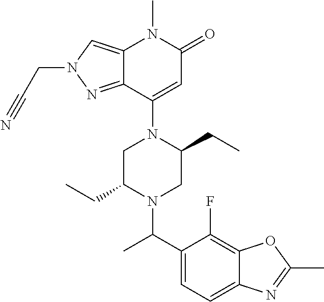 [SMILES: CC[C@H]1CN(C(C)c2ccc3nc(C)oc3c2F)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(7-fluoro-2-methylbenzo[d]oxazol-6-yl)ethan-1-ol (30 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduced pressure to afford crude product. The resulting residue was purified Prep-HPLC (Method A) to give the titled Compound A86a (18 mg), Compound A86b (14 mg) and Compound A87a (2 mg) and Compound A87b (2 mg).

Compound A86a (the earlier peak): ¹H NMR (400 MHz, CD₃OD) δ 7.92 (s, 1H), 7.52 (s, 1H), 7.45 (d, J=8.2 Hz, 1H), 5.55 (s, 1H), 5.46 (s, 2H), 4.32 (s, 1H), 3.43 (s, 3H), 3.29-3.23 (m, 2H), 3.00 (s, 2H), 2.67 (s, 3H), 2,43 (s, 1H), 2.05 (s, 1H), 1.82 (s, 1H), 1.56 (s, 2H), 1.44 (s, 3H), 1,29 (s, 1H), 0.96 (s, 3H), 0.73 (s, 3H). MS: M/e 506 (M+1)⁺.

Compound A86b (the later peak): ¹H NMR (400 MHz, CD₃OD) δ 7.93 (s, 1H), 7.60 (s, 1H), 7.43 (s, 1H), 5.55 (s, 1H), 5.47 (s, 2H), 4.16 (s, 1H), 3.43 (s, 3H), 3.28-3.12 (m, 2H), 2.80-2.68 (m, 1H), 2.66 (s, 3H), 2,43-2.30 (m, 1H), 2.03-1.49 (m, 5H), 1.39 (s, 3H), 1.35-1,23 (m, 1H), 1.09-1.01 (m, 3H), 0.69-0.53 (m, 3H). MS: M/e 506 (M+1)⁺.

Compound A87a (which was generated from the earlier peak by hydrolysis reaction): ¹H NMR (400 MHz, CD₃OD) δ 7.93 (s, 1H), 7.46 (s, 1H), 6.96 (s, 1H), 5.55 (s, 1H), 5.47 (s, 2H), 3.43 (s, 3H), 3.28-3.18 (m, 2H), 2.97 (s, 2H), 2.19 (s, 3H), 2.08-1.69 (m, 3H), 1.63-1.38 (m, 5H), 1,29 (s, 3H), 0.96 (s, 3H), 0.80 (s, 3H). MS: M/e 524 (M+1)⁺.

Compound A87b ((which was generated from the later peak by hydrolysis reaction): ¹H NMR (400 MHz, CD₃OD) δ 7.93 (s, 1H), 7.39 (s, 1H), 7.02 (s, 1H), 5.55 (s, 1H), 5.47 (s, 2H), 3.99 (s, 1H), 3.43 (s, 3H), 3.13 (s, 2H), 2.66 (s, 1H), 2,45 (s, 1H), 2.18 (s, 3H), 2.02-1.54 (m, 5H), 1.45-1.18 (m, 5H), 1.02 (s, 3H), 0.68 (s, 3H). MS: M/e 524 (M+1).

### Compound A88: 2-(7-((2S,5R)-4-(1-(benzo[c]isothiazol-5-yl)ethyl)-2,5-diethylpiperazin-1-yl)-4-methyl-5-oxo-4,5-dihydro-2H-pyrazolo[4,3-b]pyridin-2-yl)acetonitrile

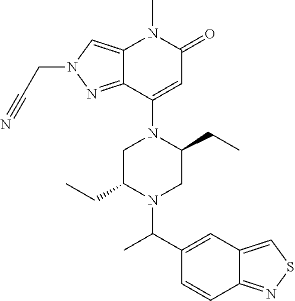 [SMILES: CC[C@H]1CN(C(C)c2ccc3nscc3c2)[C@H](CC)CN1c1cc(=O)n(C)c2cn(CC#N)nc12]

### Step A: 5-bromobenzo[c]isothiazole

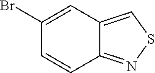

To a solution of methanesulfonamide (7.2 g, 75.3 mol) in toluene (10 mL) was added SOCl₂ (8 ml). The reaction mixture was protected by N₂ atmosphere and stirred at 120° C. for 18 h. The mixture was concentrated in vacuo to remove toluene. The resulting residue was used directly in next step.

To a solution of 4-bromo-2-methylaniline (2 g, 10.8 mol) in toluene (40 mL) was added SOCl₂ (1.4 g, 11.8 mmol) slowly at 0° C. The reaction mixture was protected by N₂ atmosphere and stirred at 120° C. for 18 hrs and cooled down to RT. The resulting residue from last step and Pyridine (1 ml) was added to this mixture. The resulting mixture was stirred at 120° C. for overnight. The mixture was added H₂O (40 ml) and extracted with EA (40 mL×3), then concentrated by using a rotary evaporator to give a residue. The resulting residue was purified by flash column chromatography to give the titled compound (800 mg, 35%). MS: M/e 214 (M+1)⁺.

### Step B: 1-(benzylisothiazolo-5-yl)ethan-1-one

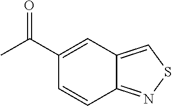

To a solution of 5-bromobenzo[c]isothiazole (428 mg, 2 mol) in toluene (10 mL) was added Pd(PPh₃)₂Cl₂ (70 mg, 0.1 mol) and tributyl(1-ethoxyvinyl)stannane (1.1 g, 3 mmol). The reaction mixture was protected by N₂ atmosphere and stirred at 90° C. for 16 hrs. The mixture was added 4N HCl in dioxane (0.5 ml) and stirred at RT for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (300 mg, 84.7%). MS: M/e 178 (M+1)”.

### Step C: 1-(benzo[c]isothiazol-5-yl)ethan-1-ol

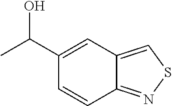

To a solution of 1-(benzo[c]isothiazol-5-yl)ethan-1-one (300 mg, 1.69 mol) in MeOH was added NaBH₄ (76 mg, 2 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (140 mg, 46.7%). MS: M/e 180 (M+1)⁺.

### Step D: 2-(7-((2S,5R)-4-(1-(benzo[c]isothiazol-5-yl)ethyl)-2,5-diethylpiperazine-1-yl)-4-methyl-5-oxo-4,5-dihydro-2H-pyrazolo[4,3-b]pyridin-2-yl)acetonitrile

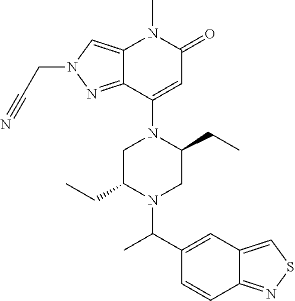 [SMILES: CC[C@H]1CN(C(C)c2ccc3nscc3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(benzo[c]isothiazol-5-yl)ethan-1-ol (70 mg, 0.4 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method B) to give the titled compound (12 mg, 24%). ¹H NMR (400 MHz, CD₃OD) δ 9.45 (dd, J=6.9, 0.9 Hz, 1H), 7.90 (d, J=2.9 Hz, 1H), 7.75 (m, 2H), 7.65 (td, J=9.3, 1.6 Hz, 1H), 5.53 (d, J=4.8 Hz, 1H), 5.44 (d, J=4.9 Hz, 2H), 3.87 (q, J=6.2 Hz, 0.5H), 3.73 (q, J=6.5 Hz, 0.5H), 3.49 (m, 1H), 3.41 (d, .=1.0 Hz, 3H), 3.30 (s, 2H), 3.11 (m, 1H), 2.89 (dd, J=12.1, 3.8 Hz, 0.5H), 2.67 (dt, J=13.3, 6.6 Hz, 0.5H), 2,42 (d, J=12.8 Hz, 1H), 2.04 (m, 1H), 1.69 (m, 3H), 1.39 (dd, J=9.6, 6.5 Hz, 3H), 1.02 (t, J=7.4 Hz, 1.5H), 0.94 (t, J=7.4 Hz, 1.5H), 0.66 (t, J=7.4 Hz, 1.5H), 0.57 (t, J=7.4 Hz, 1.5H) ppm. MS: M/e 490 (M+1)⁺.

### Compound A89: 2-(7-((2S,5R)-4-(1-(benzo[b]thiophen-6-yl)ethyl)-2,5-diethylpiperazin-1-yl)-4-methyl-5-oxo-4,5-dihydro-2H-pyrazolo[4,3-b]pyridin-2-yl)acetonitrile

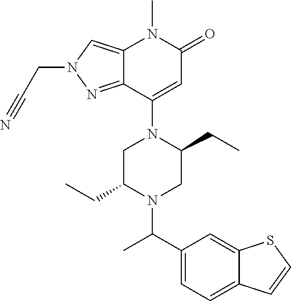 [SMILES: CC[C@H]1CN(C(C)c2ccc3ccsc3c2)[C@H](CC)CN1c1cc(=O)n(C)c2cn(CC#N)nc12]

### Step A: 1-4benzoylthiophene-6-yl)ethan-1-one

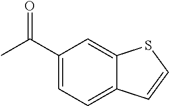

A mixture of 6-bromobenzo[b]thiophene (300 mg, 1.4 mmol), tributyl(1-ethoxyvinyl)stannane (764 mg, 2.1 mmol) and Pd(PPh₃)₂Cl₂ (99 mg, 0.14 mmol) in DMF (5 mL) was stirred at 100° C. under N₂ for 16 hours. The solution was diluted with ethyl acetate and washed with water. The organic layer was dried and added with HCl (3 mL, 3 M in ethyl acetate) in drops and the mixture was stirred at room temperature for 2 hours. The reaction was concentrated and adjusted to pH=8-9 with saturated NaHCO₃ solution, extracted with ethyl acetate. The combined organic layers were washed with brine, dried over Na₂SO₄ and the resulting residue was purified by flash column chromatography to give the titled compound (120 mg, 48%). MS: M/e 177 (M+1)⁺.-

### Step B: 1-(benzoylthiophene-6-yl)ethan-1-ol

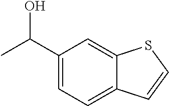

To a solution of 1-(benzo[b]thiophen-6-yl)ethan-1-one (120 mg, 0.68 mmol) in MeOH (3 mL) was added NaBH₄ (25 mg, 0.68 mmol) at room temperature and the resulting mixture was stirred at room temperature for 5 min. The reaction mixture was diluted with DCM and washed with water, dried over Na₂SO₄ and concentrated to give the titled compound (110 mg). MS: M/e 179 (M+1)⁺.

### Step C: 2-(7-((2S,5R,4-(1-(benzoylthiophene-6-yl)ethyl)-2,5-diethylpiperazin-1-vi)₄-methyl-5-oxo-4,5-dihydro-2H-pyrazolo[4,3-b]pyridin-2-yl)acetonitrile

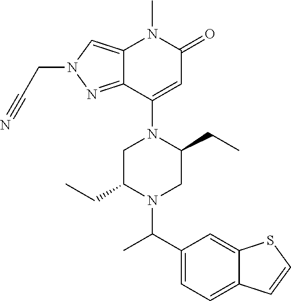 [SMILES: CC[C@H]1CN(C(C)c2ccc3ccsc3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(benzo[b]thiophen-6-yl)ethan-1-ol (50 mg, 0.28 mmol), (cyanomethyl)trimethylphosphonium iodide (111 mg, 0.45 mmol) and DIPEA(197 mg, 1.5 mmol).The resulting mixture was stirred at 105° C. overnight. The reaction solvent was removed under reduced pressure. The resulting residue was purified by flash column chromatography to give the titled compound(crude), which was further purified to give the titled compound a (7 mg) by Prep-HPLC (Method A).1H NMR (400 MHz, CD₃OD) δ 7.98-7.68 (m, 3H), 7.56-7.26 (m, 3H), 5.53 (s, 1H), 5.44 (s, 2H), 4.80-4.08 (m, 1H), 3.92-3.65 (m, 1H), 3.51 (d, J=13.0 Hz, 1H), 3.41 (s, 3H), 3.24-3.00 (m, 2H), 2.93-2.60 (m, 1H), 2,49-2.35 (m, 1H), 2.19-1.49 (m, 4H), 1.38 (dd, J=13.9, 5.8 Hz, 3H), 0.99 (d, J=27.5 Hz, 3H), 0.60 (d, J=32.9 Hz, 3H) ppm. MS: M/e 489 (M+1)⁺.

### Compound A90: 2-(7-((2S,5R)-4-(1-(benzo[d]thiazol-6-yl)ethyl)-2,5-diethylpiperazin-1-yl)-4-methyl-5-oxo-4,5-dihydro-2H-pyrazolo[4,3-b]pyridin-2-yl)acetonitrile

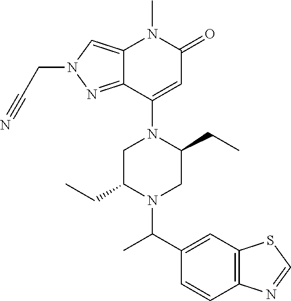 [SMILES: CC[C@H]1CN(C(C)c2ccc3ncsc3c2)[C@H](CC)CN1c1cc(=O)n(C)c2cn(CC#N)nc12]

### Step A: 1-(benzo[d]thiazol-6-yl)ethan-1-one

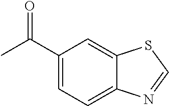

To a solution of 6-bromobenzo[d]thiazole (1 g, 5 mol) in toluene (15 mL) was added Pd(PPh₃)₂Cl₂ (350 mg, 0.5 mol) and tributyl(1-ethoxyvinyl)stannane (2.7 g, 7.5 mmol). The reaction mixture was protected by N₂ atmosphere and stirred at 90° C. for 16 hours. The mixture was added 4N HCl in 1,4-dioxane (0.5 ml) and stirred at RT for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (800 mg, 90.3%). MS: M/e 178 (M+1)⁺.

### Step B: 1-(benzo[d]thiazol-6-yl)ethan-1-ol

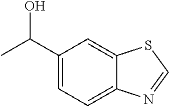

To a solution of 1-(benzo[d]thiazol-6-yl)ethan-1-one (177 mg, 1 mol) in MeOH was added NaBH₄ (38 mg, 1 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to dryness. The resulting residue was purified by flash column chromatography to give the titled compound (100 mg, 56.4%). MS: M/e 180 (M+1)⁺.

### Step C: 2-(7-((2S,5R)-4-(1-(benzo[d]thiazol-6-yl)ethyl)-2,5-diethylpiperazin-1-yl)-4-methyl-5-oxo-4,5-dihydro-2H-pyrazolo[4,3-b]pyridin-2-yl)acetonitrile

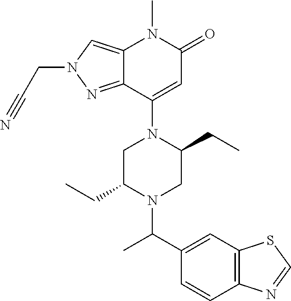 [SMILES: CC[C@H]1CN(C(C)c2ccc3ncsc3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (328 mg, 1 mmol), 1-(benzo[d]thiazol-6-yl)ethan-1-ol (260 mg, 1.45 mmol) and (cyanomethyl)trimethylphosphonium iodide (482 mg, 2 mmol) in CH₃CN (10 mL) was added DIPEA (516 mg, 4 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method A) to give the titled Compound A90 (180 mg), which was further separated into Compound A90a (47 mg, 52%) and Compound A90b (58 mg, 64%) by chiral-Prep-HPLC. The chiral separation conditions are shown below.

| Column | I-Cellulose-5 |
|---|---|
| Column Size | 20.0 mm × 250 mm, 5 um |
| Mobile Phase A | HEXANE 30% |
| Mobile Phase B | ETOH(0.2% 2M NH₃MEOH) 70% |
| Run Time | 17 min |
| Flow Rate | 18 mL/min |
| Wave Length | UV 200 nm and 310 nm |
| Temperature | RT |
| Prep-HPLC Equipment | Prep-HPLC-Gilson |

Compound A90: ¹H NMR (500 MHz, CD₃OD) δ 9.21 (s, 1H), 8.06 (m, 2H), 7.91 (s, 1H), 7.63 (dd, .=8.5, 1.4 Hz, 1H), 5.56 (s, 1H), 5.45 (s, 2H), 3.94 (q, J=6.4 Hz, 1H), 3.43 (s, 3H), 3.31 (s, 3H), 3.06 (d, J=10.9 Hz, 1H), 2.91 (dd, J=12.0, 3.8 Hz, 1H), 2,43 (d, J=8.7 Hz, 1H), 2.15 (m, 1H), 1.85 (m, 1H), 1.56 (m, 2H), 1.42 (d, J=6.5 Hz, 3H), 0.97 (t, J=7.4 Hz, 3H), 0.67 (t, J=7.4 Hz, 3H) ppm. MS: M/e 490 (M+1)⁺.

Compound A90b (the earlier peak): ¹H NMR (500 MHz, CD₃OD) δ 9.20 (s, 1H), 8.11 (s, 1H), 8.02 (d, J=8.5 Hz, 1H), 7.92 (s, 1H), 7.65 (dd, J=8.5, 1.5 Hz, 1H), 5.55 (s, 1H), 5.46 (d, J=5.8 Hz, 2H), 3.80 (q, J=6.6 Hz, 1H), 3.54 (d, J=12.9 Hz, 1H), 3.43 (s, 3H), 3.32 (s, 2H), 3.19 (m, 1H), 2.68 (dd, J=12.2, 3.6 Hz, 1H), 2.37 (d, J=12.1 Hz, 1H), 2.00 (dd, J=14.4, 7.0 Hz, 1H), 1.65 (m, 3H), 1.39 (d, J=6.6 Hz, 3H), 1.04 (t, J=7.3 Hz, 3H), 0.57 (t, J=7.4 Hz, 3H) ppm. MS: M/e 490 (M+1)⁺.

Compound A90b (the later peak): ¹H NMR (500 MHz, CD₃OD) δ 9.20 (d, J=5.8 Hz, 1H), 8.05 (m, 2H), 7.92 (d, J=3.6 Hz, 1H), 7.65 (d, J=8.7 Hz, 1H), 5.55 (d, J=4.2 Hz, 1H), 5.46 (d, J=5.5 Hz, 2H), 3.94 (d, J=6.5 Hz, 0.3H), 3.80 (d, J=6.7 Hz, 0.7H), 3.54 (d, J=13.9 Hz, 0.6H), 3.43 (s, 3.4H), 3.31 (s, 2H), 3.19 (m, 1H), 3.06 (d, J=12.2 Hz, 0.4H), 2.68 (d, J=8.5 Hz, 0.6H), 2.39 (t, J=15.2 Hz, 1H), 1.89 (m, 1H), 1.65 (m, 3H), 1.40 (dd, J=14.1, 6.5 Hz, 3H), 1.04 (t, J=7.4 Hz, 2H), 0.97 (s, 1H), 0.67 (t, J=7.3 Hz, 1H), 0.57 (t, J=7.4 Hz, 2H) ppm. MS: M/e 490 (M+1)⁺.

### Compound A91: 2-(7-((2S,5R)-2,5-diethyl-4-(1-(2-methylbenzo[d]thiazol-6-yl)ethyl)piperazin-1-yl)-4-methyl-5-oxo-4,5-dihydro-2H-pyrazolo[4,3-b]pyridin-2-yl)acetonitrile

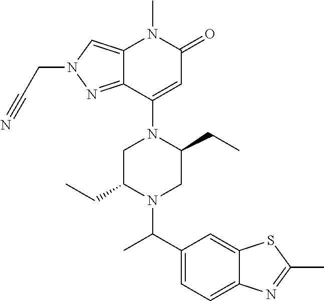 [SMILES: CC[C@H]1CN(C(C)c2ccc3nc(C)sc3c2)[C@H](CC)CN1c1cc(=O)n(C)c2cn(CC#N)nc12]

### Step A: 1-(2-methylbenzo[d]thiazol-6-yl)ethan-1-one

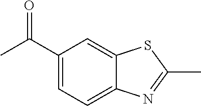

To a solution of 6-bromo-2-methylbenzo[d]thiazole (912 mg, 4 mol) in toluene (15 mL) was added Pd(PPh₃)₂Cl₂ (140 mg, 0.2 mol) and tributyl(1-ethoxyvinyl)stannane (2.2 g, 6 mmol). The reaction mixture was protected by N₂ atmosphere and stirred at 90° C. for 16 hrs. The mixture was added 4N HCl in dioxane (0.5 ml) and stirred at rt for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (450 mg, 58.9%). MS: M/e 192 (M+1).

### Step B: 1-(2-methylbenzo[d]thiazol-6-yl)ethan-1-ol

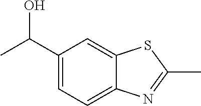

To a solution of 1-(2-methylbenzo[d]thiazol-6-yl)ethan-1-one (450 mg, 2,5 mol) in MeOH was added NaBH₄ (76 mg, 2 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (320 mg, 71%). MS: M/e 194 (M+1)⁺.

### Step C: 2-(7-((2S,5R)-2,5-diethyl-4-(142-methylbenzo[d]thiazol-6-yl)ethyl)piperazin-1-yl)-4methyl-5-oxo-4,5-dihydro-2H-pyrazolo[4,3-b]pyridin-2-yl)acetonitrile

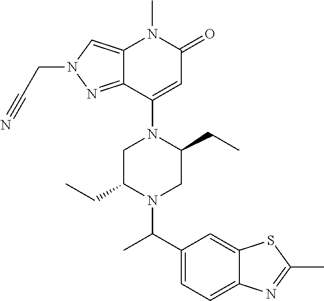 [SMILES: CC[C@H]1CN(C(C)c2ccc3nc(C)sc3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(2-methylbenzo[d]thiazol-6-yl)ethan-1-ol (53 mg, 0.3 mmol) and (cyanomethyl)trimethylphosphonium iodide (120 mg, 0.5 mmol) in CH₃CN (10 mL) was added DIPEA (129 mg, 1 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method A) to give the Compound A91a (3 mg) and Compound A91b (3 mg).

Compound A91a (the earlier peak): ¹H NMR (500 MHz, CD₃OD) δ 7.91 (s, 2H), 7.86 (d, J=8.4 Hz, 1H), 7.54 (d, J=8.3 Hz, 1H), 5.55 (s, 1H), 5.45 (s, 2H), 3.89 (d, 0.1=6.6 Hz, 1H), 3.43 (s, 3H), 3.32 (s, 3H), 3.05 (d, J=11.8 Hz, 1H), 2.90 (d, J=8.2 Hz, 1H), 2.83 (s, 3H), 2,41 (s, 1H), 2.13 (dd, J=14.5, 7.2 Hz, 1H), 1.84 (m, 1H), 1.55 (m, 2H), 1.40 (d, J=6.4 Hz, 3H), 0.97 (t, J=7.3 Hz, 3H), 0.67 (t, J=7.3 Hz, 3H) ppm. MS: M/e 504 (M+1)⁺.

Compound A91b (the later peak): ¹H NMR (400 MHz, CD₃OD) δ 7.94 (d, J=15.6 Hz, 2H), 7.83 (d, J=8.4 Hz, 1H), 7.56 (d, J=8.3 Hz, 1H), 5.54 (s, 1H), 5.46 (s, 2H), 3.75 (s, 1H), 3.52 (d, J=13.6 Hz, 1H), 3.43 (s, 3H), 3.32 (s, 2H), 3.19 (d, J=9.5 Hz, 1H), 2.82 (s, 3H), 2.66 (d, 0.1=12.1 Hz, 1H), 2.37 (d, 0.1=12.2 Hz, 1H), 2.02 (s, 1H), 1.63 (d, J=15.2 Hz, 3H), 1.37 (d, 0.1=5.8 Hz, 3H), 1.04 (s, 3H), 0.57 (s, 3H) ppm. MS: We 504 (M+1)⁺.

### Compound A92: 2-(7-((2S,5R)-4-(1-(benzo[d]thiazol-6-yl)ethyl)-5-ethyl-2-methylpiperazin-1-yl)-4-methyl-5-oxo-4,5-dihydro-2H-pyrazolo[4,3-b]pyridin-2-yl)acetonitrile

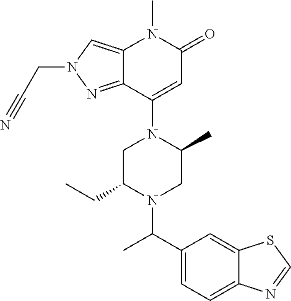 [SMILES: CC[C@@H]1CN(c2cc(=O)n(C)c3cn(CC#N)nc23)[C@@H](C)CN1C(C)c1ccc2ncsc2c1]

To a solution of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (314 mg, 1 mmol), 1-(benzo[d]thiazol-6-yl)ethan-1-ol (358 mg, 2 mmol) and (cyanomethyl)trimethylphosphonium iodide (723 mg, 3 mmol) in CH₃CN (10 mL) was added DIPEA (516 mg, 4 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method B) to give the Compound A92a (67 mg) and Compound A92b (74 mg).

Compound A92a (the earlier peak): ¹H NMR (500 MHz, CD₃OD) δ 9.20 (s, 1H), 8.05 (dd, J=11.5, 5.0 Hz, 2H), 7.90 (s, 1H), 7.62 (dd, J=8.5, 1.6 Hz, 1H), 5.56 (s, 1H), 5.43 (s, 2H), 3.93 (t, J=6.5 Hz, 1H), 3.41 (s, 3H), 3.30 (s, 3H), 2.98 (dd, J=11.8, 4.2 Hz, 1H), 2.87 (dd, J=11.9, 2,4 Hz, 1H), 2,42 (s, 1H), 1.56 (m, 2H), 1.40 (dd, J=6.5, 3.0 Hz, 6H), 0.70 (t, J=7.4 Hz, 3H) ppm. MS: M/e 476 (M+1)⁺.

Compound A92b (the later peak): ¹H NMR (400 MHz, CD₃OD) δ 9.18 (s, 1H), 8.09 (d, J=1.4 Hz, 1H), 8.00 (d, J=8.5 Hz, 1H), 7.91 (s, 1H), 7.66 (d, J=1.6 Hz, 1H), 5.55 (s, 1H), 5.45 (s, 2H), 3.79 (d, J=6.5 Hz, 1H), 3.55 (d, J=11.0 Hz, 1H), 3.42 (s, 3H), 3.30 (s, 2H), 3.18 (d, J=9.8 Hz, 1H), 2.76 (dd, J=12.1, 4.1 Hz, 1H), 2.23 (d, J=10.5 Hz, 1H), 1.66 (m, 2H), 1.39 (t, J=6.1 Hz, 3H), 1,20 (d, J=6.5 Hz, 3H), 1.05 (t, J=7.4 Hz, 3H) ppm. MS: Me 476 (M+1)⁺.

### Compound A93: 2-(7-((2S,5R)-4-(1-(benzo[d]thiazol-6-yl)ethyl)-2,5-dimethylpiperazin-1-yl)-4-methyl-5-oxo-4,5-dihydro-2H-pyrazolo[4,3-b]pyridin-2-yl)acetonitrile

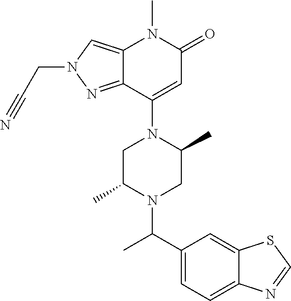 [SMILES: CC(c1ccc2ncsc2c1)N1C[C@H](C)N(c2cc(=O)n(C)c3cn(CC#N)nc23)C[C@H]1C]

### Step A: 7-((2S,5R)-(1-benzo[d]thiazol-6-yl)ethyl-2,5-dimethylpiperazin-1-yl)-4-methyl-2-(tetrahydro-2H-pyran-2-yl)-2,4-dihydro-5H-pyrazolo[4,3-b]pyridin-5-one

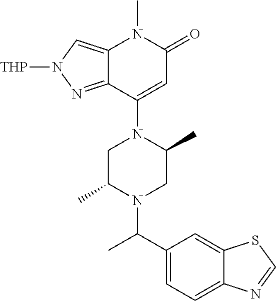

A mixture of 7-((2S,5R)-2,5-dimethylpiperazin-1-yl-4-methyl-2-(tetrahydro-2H-pyran-2-yl)-2,4-dihydro-5H-pyrazolo[4,3-b]pyridin-5-one (103 mg, 0.33 mmol), 1-(benzo[d]thiazol-6-yl)ethan-1-ol(80 mg, 0.45 mmol), (cyanomethyl) trimethylphosphonium iodide (218 mg, 0.9 mmol) and DIPEA (193 mg, 1.5 mmol) in MeCN (2 mL) was stirred at 100° C. overnight. The reaction mixture was diluted with EtOAc (20 mL), washed with water (10 mL×2), dried over Na₂SO₄ and concentrated to dryness. The resulting residue was purified by flash column chromatography (MeOH: DCM=0-10%/ in 25 minutes) to give the titled compound (60 mg, 36%). MS: M/e 507 (M+1).

### Step B: 7-((2S,5R)-4-(1-(benzodithiazol-6-yl)ethyl)-2,5-dimethylpiperazin-1-yl)-4-methyl-2,4-dihydro-5H-pyrazolo[4,3-b]pyridin-5-one

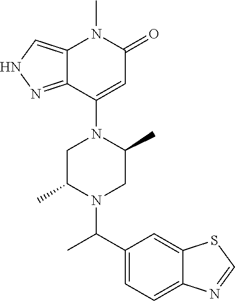 [SMILES: CC(c1ccc2ncsc2c1)N1C[C@H](C)N(c2cc(=O)n(C)c3c[nH]nc23)C[C@H]1C]

To a mixture of 7-((2S,5R)-4-(1-(benzo[d]thiazol-6-yl)ethyl)-2,5-dimethylpiperazin-1-yl)-4-methyl-2-(tetrahydro-2H-pyran-2-yl)-2,4-dihydro-5H-pyrazolo[4,3-b]pyridin-5-one (50 mg) in MeOH (2 mL) was added HCl(g) (4 M in dioxane, 2 ml). The reaction was stirred at room temperature overnight. The solvent was removed under reduced pressure. The resulting residue (42 mg, crude) was used directly for next step without further purification. MS: M/e 423 (M+1)⁺.

### Step C: 2-(7-((2S,5R′)-4-(1-benzo[d]thiazol-6-yl)ethyl)-2,5-dimethylpiperazin-1-yl)-4-methyl-5-oxo-4,5-dihydro-2H-pyrazolo[4,3-b]pyridin-2-yl)acetonitrile

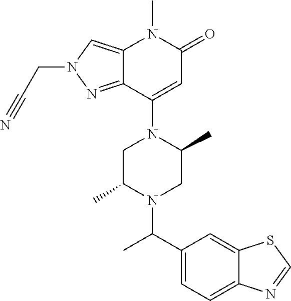 [SMILES: CC(c1ccc2ncsc2c1)N1C[C@H](C)N(c2cc(=O)n(C)c3cn(CC#N)nc23)C[C@H]1C]

To a mixture of 7-((2S,5R)-4-(1-(benzo[d]thiazol-6-yl)ethyl)-2,5-dimethylpiperazin-1-yl)-4-methyl-2,4-dihydro-5H-pyrazolo[4,3-b]pyridin-5-one (42 mg, crude) in DMF (2 mL) was added potassium carbonate (55 mg, 0.4 mmol) and 2-chloroacetonitrile (15 mg, 0.2 mmol ). The resulting mixture was stirred at room temperature for overnight. The reaction was diluted with water (20 ml), extracted with EtOAc(10 ml×3), washed with brine, dried over Na₂SO₄, filtered, and concentrated to dryness. The resulting residue was purified by flash column chromatography (MeOH: DCM=0-10% in 25 minutes) to give the titled compound (29 mg, 63%). ¹H NMR (400 MHz, DMSO-d₆) δ 9.31 (d, J=3.9 Hz, 1H), 8.15-7.91 (m, 3H), 7.62-7.47 (m, 1H), 5.58 (s, 2H), 5.36 (d, J=7.2 Hz, 1H), 5.01-4.25 (m, 1H), 3.80-3.57 (m, 1H), 3.57-3.41 (m, 2H), 3.24 (s, 3H), 2.94-2,59 (m, 2H), 2.05 (d, J=12.0 Hz, 1H), 1.36-1,21 (m, 4H), 1.05 (d, J=6.3 Hz, 3H), 0.90 (d, J=6.4 Hz, 2H) ppm. MS: M/e 462 (M+1)⁺.

### Compound A95: 7-((2S,5R)-4-(1-(benzo[d]thiazol-6-yl)ethyl)-2,5-diethylpiperazin-1-yl)-2-(cyanomethyl)-4-methyl-5-oxo-4,5-dihydro-2H-pyrazolo[4,3-b]pyridine-6-carbonitrile

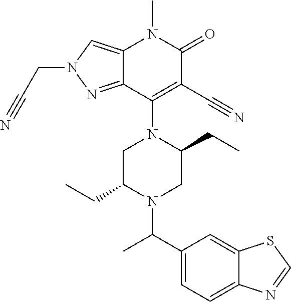 [SMILES: CC[C@H]1CN(C(C)c2ccc3ncsc3c2)[C@H](CC)CN1c1c(C#N)c(=O)n(C)c2cn(CC#N)nc12]

### Step A: 2-(7-((2S,5R)-4-(1-(benzo[d]thiazol-6-yl)ethyl-2,5-diethylpiperazin-1-yl-6-bromo-4-methyl-5-oxo-4,5-dihydro-2H-pyrazolo[4,3-b]pyridin-2-ylacetonitrile

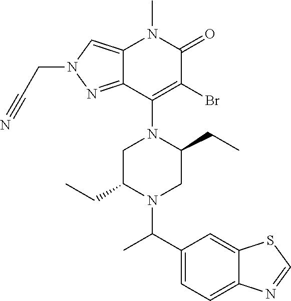 [SMILES: CC[C@H]1CN(C(C)c2ccc3ncsc3c2)[C@H](CC)CN1c1c(Br)c(=O)n(C)c2cn(CC#N)nc12]

To a mixture of 2-(7-((2S,5R)-4-(1-(benzo[d]thiazol-6-yl)ethyl)-2,5-diethylpiperazin-1-yl)-4-methyl-5-oxo-4,5-dihydro-2H-pyrazolo[4,3-b]pyridin-2-yl)acetonitrile (140 mg, 0.286 mmol) in CH₃CN (3 mL) was added a solution of NBS (51 mg, 0.286 mmol) in CH₃CN (1 mL). The mixture was stirred at room temperature for 16 hours. The reaction was diluted with water, extracted with EA (60 mL×2), washed with brine, dried over Na₂SO₄, filtered, and concentrated to dryness. The resulting residue was purified by combi flash (DCM:MeOH=20:1) to give the titled Compound A94 (70 mg, 43%). ¹H NMR (400 MHz, CD₃OD) 5 9.20 (s, 0.5 H), 9.19 (s, 0.5H), 8.13-7.99 (m, 2H), 7.96 (s, 1H), 7.70-7.60 (m, 1H), 5.50 (s, 1H), 5.49 (s, 1H), 4.55-4.40 (m, 0.5H), 4.26-4.14 (in, 0.5H), 4.06-3.92 (m, 1H), 3.86-3.65 (m, 1.5H), 3.53 (s, 1.5H), 3.52 (s, 1.5H), 3.48-3.40 (in, 0.5H), 3.18-3.04 (m, 1H), 2.96-2.86 (m, 0.5H), 2,42-1.89 (m, 2,5H), 1.85-1.50 (m, 3H), 1.41 (d, J=6.4 Hz, 1.5H), 1.37 (d, J=6.4 Hz, 1.5H), 0.86 (t, J=7.6 Hz, 1.5H), 0.78 (t, J=7.6 Hz, 1.5H), 0.60 (t, J=7.6 Hz, 1.5H), 0.45 (t, J=7.6 Hz, 1.5H) ppm. MS: M/e 568 (M+1)⁺.

### Step B: 7-((2S,5R)-4-(1-(benzo[d]thiazol-6-yl)ethyl)-2,5-diethylpiperazin-1-yl)-2-(cyanomethyl)-4-methyl-5-oxo-4,5-dihydro-2H-pyrazolo[4,3-b]pyridine-6-carbonitrile

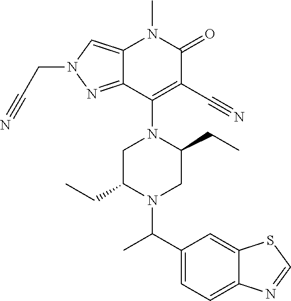 [SMILES: CC[C@H]1CN(C(C)c2ccc3ncsc3c2)[C@H](CC)CN1c1c(C#N)c(=O)n(C)c2cn(CC#N)nc12]

11 To a mixture of 2-(7-((2S,5R)-4-(1-(benzo[d]thiazol-6-yl)ethyl)-2,5-diethylpiperazin-1-yl)-6-broo-4-methyl-5-oxo-4,5-dihydro-2H-pyrazolo[4,3-b]pyridin-2-yl)acetonitrile (62 mg, 0.11 mmol) in DMF (2 mL) was added Zn(CN)₂ (39 mg (m, 0.33 mmol) and Pd(PPh₃)₄ (38 mg, 0.033 mmol). The mixture was stirred at 100° C. for 16 hours under N₂. The reaction was cooled to room temperature, diluted with water, extracted with EA(60 mL×2), washed with brine, dried over Na₂SO₄, filtered, and concentrated to dryness. The resulting residue was purified by Prep-HPLC (Method A) to give the titled compound(38 mg, 67%). ¹H NMR (400 MHz, CD₃OD) δ 9.21 (s, 1H), 8.13-8.01 (m, 2H), 8.00 (s, 1H), 7.68-7.62 (m, 1H), 5.51 (s, 2H), 4.05-3.70 (m, 2,5H), 3.42-3.43 (m, 3H), 3.31-3.28 (m, 2H), 3.27-2.85 (m, 2H), 2,52-1.95 (m, 2,5H), 1.70-1.50 (m, 2H), 1.44-1.36 (m, 3H), 0.95-0.84 (m, 3H), 0.75-0.50 (m, 3H) ppm. MS: M/e 515 (M+1)⁺.

### Compound A96: 2-(7-((2S,5R)-2,5-diethyl-4-(1-(2-ethylbenzo[d]thiazol-6-yl)ethyl)piperazin-1-yl)-4-methyl-5-oxo-4,5-dihydro-2H-pyrazolo[4,3-b]pyridin-2-yl)acetonitrile

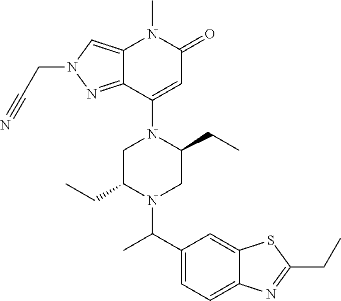 [SMILES: CCc1nc2ccc(C(C)N3C[C@H](CC)N(c4cc(=O)n(C)c5cn(CC#N)nc45)C[C@H]3CC)cc2s1]

Step A: 6-bromo-2-ethylbenzo[d]thiazole

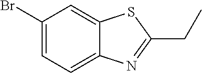

To a solution of LDA (2 M in THF, 1,2 ml, 2,4 mmol) in THF (6 ml) was added dropwise to a solution of 6-bromo-2-methylbenzo[d]thiazole (456 mg, 2 mmol) in THF (4 ml) which was cooled to −78° C., and the mixture was stirred at the same temperature for 30 min. Methyl iodide (1.4 g, 10 mmol) was added, and the mixture was further stirred for 4 hrs. To the reaction mixture was quenched with saturated ammonium chloride (40 ml), and the mixture was extracted with EtOAc (20 ml×2). The organic layer was dried over anhydrous sodium sulfate, and the solvent was evaporated under reduced pressure. The resulting residue was purified by flash column chromatography (EtOAc: PE=0-50% in 20 minutes) to give the titled compound (420 mg, 87%).

¹H NMR (400 MHz, CDCl₃) δ 7.99 (t, J=11.6 Hz, 1H), 7.80 (t, J=13.4 Hz, 1H), 7.55 (dd, J=8.7, 1.8 Hz, 1H), 3.14 (q, J=7.6 Hz, 2H), 1.47 (t, J=7.6 Hz, 3H) ppm. MS: M/e 242 (M+1)⁺.

### Step B: 1-(2-ethylbenzo[d]thiazol-6-yl)ethan-1-one

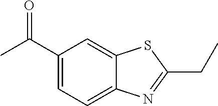

A mixture of 6-bromo-2-ethylbenzo[d]thiazole (420 mg, 1.74 mmol), tributyl(1-ethoxyvinyl)stannane (943 mg, 2.6 mmol) and Pd(PPh₃)₂Cl₂ (120 mg, 0.17 mmol) in toluene (5 mL) was stirred at 100° C. under N₂ for 16 hours. To the resulting solution was added HCl (0.5 mL, con.) in drops and the mixture was stirred at room temperature for 30 minutes. The mixture was treated with saturated NaHCO₃ aq. (20 ml), extracted with EtOAc(10 ml×3), washed with brine (20 mL), dried and concentrated to dryness. The resulting residue was purified by flash column chromatography (EtOAc: PE=0-50% in 20 minutes) to give the titled compound (230 mg, 64%). MS: M/e 206 (M+1)⁺.

### Step C: 1-(2-ethylbenzo[d]thiazol-6-yl)ethan-1-ol

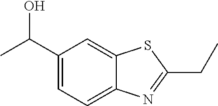

To a solution of 1-(2-ethylbenzo[d]thiazol-6-yl)ethan-1-one (230 mg, 1.12 mmol) in MeOH (5 mL) was added NaBH₄ (34 mg, 0.9 mmol) at room temperature and the mixture was stirred at room temperature for 30 minutes. The resulting mixture was treated with water(20 ml), extracted with EtOAc (10 mL×2). The combined organic layers were dried over Na₂SO₄ and concentrated to dryness. The resulting residue (200 mg, crude) was used in the next step directly. MS: M/e 208 (M+1)⁺.

### Step D: 2-(7-((2S,5R)-2,5-diethyl-4-(142-ethylbenzo[d]thiazol-6-yl)ethyl)piperazin-1-yl)-4-methyl-5-oxo-4,5-dihydro-2H-pyrazolo[4,3-b]pyridin-2-yl)acetonitrile

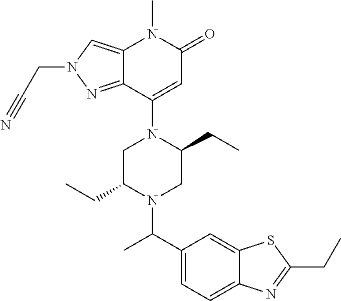 [SMILES: CCc1nc2ccc(C(C)N3C[C@H](CC)N(c4cc(=O)n(C)c5cn(CC#N)nc45)C[C@H]3CC)cc2s1]

A mixture of 1-(2-ethylbenzo[d]thiazol-6-yl)ethan-1-ol (41 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (97 mg, 0.4 mmol) and DIPEA (51 mg, 0.4 mmol) in MeCN (2 mL) was stirred at 100° C. overnight. The reaction mixture was diluted with EtOAc (20 mL), washed with water (10 mL×2), dried over Na₂SO₄ and concentrated to dryness. The resulting residue was purified by Prep-TLC (MeOH:DCM=1:15) to give the titled compound (30 mg, crude), which was further separated into Compound A96a (0.9 mg) and Compound A96b (1.6 mg) by Prep- HPLC (Method B).

Compound A96a (the earlier peak) ¹H NMR (400 MHz, DMSO-d₆) δ 7.97 (d, J=14.1 Hz, 2H), 7.87 (d, J=8.3 Hz, 1H), 7.46 (d, J=8.3 Hz, 1H), 5.58 (s, 2H), 5.37 (s, 1H), 3.82 (d, J=6.3 Hz, 1H), 3.36 (d, J=13.2 Hz, 1H), 3.25 (s, 3H), 3.09 (dd, J=15.0, 7.5 Hz, 3H), 2.92 (d, J=11.8 Hz, 1H), 2.83-2.64 (m, 1H), 2,51 (d, J=11.9 Hz, 1H), 2.29 (d, J=8.6 Hz, 1H), 2.13-1.84 (m, 1H), 1.79-1.54 (m, 1H), 1.45 (d, J=6.5 Hz, 2H), 1.39-1,23 (m, 6H), 0.85 (t, J=7.0 Hz, 3H), 0.56 (s, 3H) ppm. MS: M/e 518 (M+1)⁺.

Compound A96b (the later peak) ¹H NMR (400 MHz, DMSO-d₆) δ 7.97 (d, J=11.1 Hz, 2H), 7.85 (d, J=8.4 Hz, 1H), 7.48 (d, J=8.3 Hz, 1H), 5.59 (s, 2H), 5.36 (s, 1H), 3.69 (d, J=6.2 Hz, 1H), 3.36 (d, J=17.2 Hz, 2H), 3.25 (s, 3H), 3.09 (dd, J=15.0, 7.4 Hz, 3H), 2,52 (s, 1H), 2.22 (d, J=11.8 Hz, 1H), 1.92 (t, J=22.1 Hz, 1H), 1.53 (dd, J=27.0, 17.6 Hz, 3H), 1.35 (t, J=7.5 Hz, 4H), 1,25 (t, J=12.9 Hz, 3H), 0.91 (dd, J=28.7, 21.5 Hz, 3H), 0.46 (s, 3H) ppm. MS: M/e 518 (M+1)⁺.

### Compound A97: 2-(7-((2S,5R)-2,5-diethyl-4-(1-(thiazolo[4,5-c]pyridin-6-yl)ethyl)piperazin-1-yl)-4-methyl-5-oxo-4,5-dihydro-2H-pyrazolo[4,3-b]pyridin-2-yl)acetonitrile

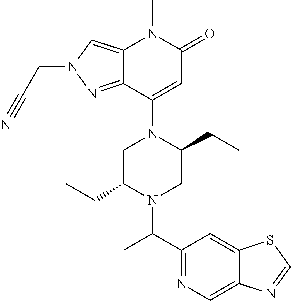 [SMILES: CC[C@H]1CN(C(C)c2cc3scnc3cn2)[C@H](CC)CN1c1cc(=O)n(C)c2cn(CC#N)nc12]

### Step A: 1-(thiazolo[4.5-c]pyridin-6-yl)ethan-1-one

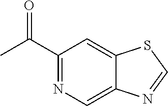

A mixture of 6-bromothiazolo[4,5-c]pyridine (250 mg, 1.17 mmol), tributyl(1-ethoxyvinyl)stannane (634 mg, 1.75 mmol) and Pd(PPh₃)₂Cl₂ (81 mg, 0.11 mmol) in DMF (3 mL) was stirred at 100° C. under N₂ for 16 hours. The solution was diluted with ethyl acetate and washed with water. The organic layer was dried and added with HCl (3 mL, 3 M in ethyl acetate) in drops and the mixture was stirred at room temperature for 2 hours. The reaction was concentrated and adjusted to pH=8-9 with saturated NaHCO₃ solution, extracted with ethyl acetate. The combined organic layers were washed with brine, dried over Na₂SO₄ and the resulting residue was purified by flash column chromatography to give the titled compound (110 mg, 52%). MS: M/e 179 (M+1)⁺

### Step B: 1-(thiazolo[4.5-c]pyridin-6-ylethan-1-ol

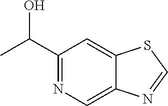

To a solution of 1-(thiazolo[4,5-c]pyridin-6-yl)ethan-1-one (110 mg, 0.61 mmol) in MeOH (3 mL) was added NaBH₄ (23 mg, 0.61 mmol) at room temperature and the resulting mixture was stirred at room temperature for 5 min. The reaction mixture was diluted with DCM and washed with water, dried over Na₂SO₄ and concentrated to give the titled compound (70 mg). MS: M/e 181 (M+1)⁺.

### Step C: 2474 (2S,5R)-2,5-diethyl-4-41-(thiazolo[4.5-c]pyridin-6-yl)ethyl)piperazin-1-yl)-4-methyl-5-oxo-4,5-dihydro-2H-pyrazolo[4,3-b]pyridin-2-yl)acetonitrile

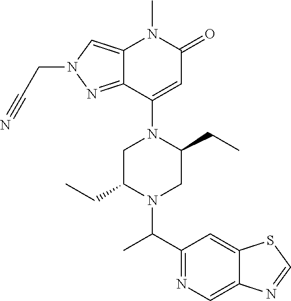 [SMILES: CC[C@H]1CN(C(C)c2cc3scnc3cn2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(thiazolo[4,5-c]pyridin-6-yl)ethan-1-ol (70 mg, 0.38 mmol), (cyanomethyl)trimethylphosphonium iodide (111 mg, 0.45 mmol) and DIPEA(197 mg, 1.5 mmol).The resulting mixture was stirred at 105° C. overnight. The reaction solvent was removed under reduce pressure. The resulting residue was purified by flash column chromatography to give the titled compound(crude), which was further purified to give the titled compound(1 mg) by Prep-HPLC (Method A). ¹H NMR (400 MHz, CD₃OD) δ 9.34 (d, J=1.7 Hz, 1H), 9.24 (d, J=4.5 Hz, 1H), 8.36 (d, J=20.3 Hz, 1H), 7.92 (d, J=3.2 Hz, 1H), 5.56 (s, 1H), 5.46 (d, J=4.1 Hz, 2H), 4.78-4.60 (m, 1H), 4.23-3.57 (m, 2H), 3.43 (s, 3H), 3.20 (dd, J=19.7, 10.2 Hz, 2H), 3.10-2.75 (m, 1H), 2,48-2.25 (m, 1H), 2.22-1.56 (m, 4H), 1.51-1.43 (m, 3H), 1.12-0.75 (m, 3H), 0.77-0.53 (m, 3H) ppm. MS: M/e 491 (M+1)⁺.

### Compound A98: 2-(7-((2S,5R)-2,5-diethyl-4-(1-(thiazolo[4,5-b]pyridin-6-yl)ethyl)piperazin-1-yl)-4-methyl-5-oxo-4,5-dihydro-2H-pyrazolo[4,3-b]pyridin-2-yl)acetonitrile

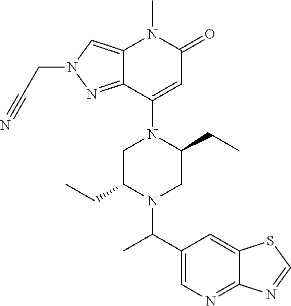 [SMILES: CC[C@H]1CN(C(C)c2cnc3ncsc3c2)[C@H](CC)CN1c1cc(=O)n(C)c2cn(CC#N)nc12]

### Step A: 1-(thiazolo[4.5-b]pyridin-6-yl)ethan-1-one

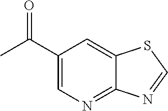

A mixture of 6-bromothiazolo[4,5-b]pyridine (250 mg, 1.17 mmol), tributyl(1-ethoxyvinyl)stannane (634 mg, 1.75 mmol) and Pd(PPh₃)₂Cl₂ (81 mg, 0.11 mmol) in DMF (3 mL) was stirred at 100° C. under N₂ for 16 hours. The solution was diluted with ethyl acetate and washed with water. The organic layer was dried and added with HCl (3 mL, 3 M in ethyl acetate) in drops and the mixture was stirred at room temperature for 2 hours. The reaction was concentrated and adjusted to pH=8-9 with saturated NaHCO₃ solution, extracted with ethyl acetate. The combined organic layers were washed with brine, dried over Na₂SO₄ and the resulting residue was purified by flash column chromatography to give the titled compound (80 mg, 38%). MS: M/e 179 (M+1)⁺

### Step B: 1-(thiazolo[4.5-b]pyridin-6-yl)ethan-1-ol

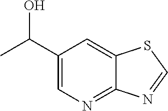

To a solution of 1-(thiazolo[4,5-b]pyridin-6-yl)ethan-1-one (80 mg, 0.44 mmol) in MeOH (3 mL) was added NaBH₄ (23 mg, 0.44 mmol) at room temperature and the resulting mixture was stirred at room temperature for 5 min. The reaction mixture was diluted with DCM and washed with water, dried over Na₂SO₄ and concentrated to give the titled compound (50 mg). MS: M/e 181 (M+1)⁺.

### Step C: 2-(7-((2S,5R)-2,5-diethyl-4-(1-(thiazolo[4.5-b]pyridin-6-yl)ethyl)piperazin-1-yl)-4-methyl-5-oxo-4,5-dihydro-2H-pyrazolo[4,3-b]pyridin-2-yl)acetonitrile

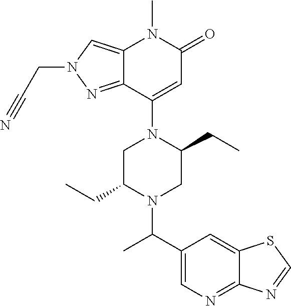 [SMILES: CC[C@H]1CN(C(C)c2cnc3ncsc3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(thiazolo[4,5-b]pyridin-6-yl)ethan-1-ol (50 mg, 0.3 mmol), (cyanomethyl)trimethylphosphonium iodide (111 mg, 0.45 mmol) and DIPEA(197 mg, 1.5 mmol).The resulting mixture was stirred at 105° C. overnight. The reaction solvent was removed under reduced pressure. The resulting residue was purified by flash column chromatography to give the titled compound(crude), which was further purified to give the titled Compound A98a (0.7 mg) and Compound A98b (0.8 mg) by Prep-HPLC (Method A).

Compound A98a (the earlier peak): ¹H NMR (400 MHz, CD₃OD) δ 9.52 (s, 1H), 8.72 (s, 1H), 8.62 (s, 1H), 7.90 (s, 1H), 5.55 (s, 1H), 5.44 (s, 2H), 4.81-4.32 (m, 2H), 4.06 (d, J=6.4 Hz, 1H), 3.41 (s, 3H), 3.32 (d, J=14.4 Hz, 1H), 2.99 (dd, J=43.1, 12,4 Hz, 2H), 2.39 (s, 1H), 1.97 (dd, J=102.6, 7.1 Hz, 2H), 1.69-1.53 (m, 2H), 1.45 (d, J=6.4 Hz, 3H), 0.94 (t, J=7.1 Hz, 3H), 0.71 (t, 0.1=7.0 Hz, 3H) ppm. MS: M/e 491 (M+1)⁺.

Compound A98b (the later peak): ¹H NMR (400 MHz, CD₃OD) δ 9.51 (s, 1H), 8.76 (s, 1H), 8.63 (s, 1H), 7.91 (s, 1H), 5.54 (s, 1H), 5.45 (s, 2H), 4.83-4.02 (m, 2H), 3.89 (d, J=6.2 Hz, 1H), 3.54 (d, J=12.6 Hz, 1H), 3.41 (s, 3H), 3.20 (d, J=9.9 Hz, 1H), 2.73 (d, J=11.0 Hz, 1H), 2.31 (d, J=12.2 Hz, 1H), 2.01-1.49 (m, 4H), 1.42 (d, J=6.2 Hz, 3H), 1.03 (t, J=7.0 Hz, 3H), 0.55 (t, J=7.0 Hz, 3H) ppm. MS: M/e 491 (M+1)⁺.

### Compound A99: 2-(7-((2S,5R)-2,5-diethyl-4-(1-(thiazolo[5,4-b]pyridin-5-yl)ethyl)piperazin-1-yl)-4-methyl-5-oxo-4,5-dihydro-2H-pyrazolo[4,3-b]pyridin-2-yl)acetonitrile

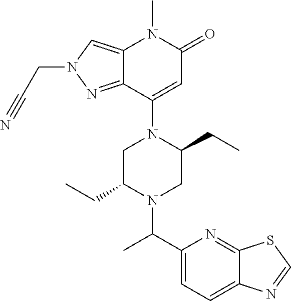 [SMILES: CC[C@H]1CN(C(C)c2ccc3ncsc3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (250 mg, 0.76 mmol) in CH₃CN (8 mL) and was added 1-(thiazolo[5,4-b]pyridin-5-yl)ethan-1-ol (274 mg, 1.52 mmol), (cyanomethyl)trimethylphosphonium iodide (556 mg, 2.28 mmol) and DIPEA(0.61 g, 4.7 mmol).The resulting mixture was stirred at 105° C. overnight. The reaction solvent was removed under reduced pressure. The resulting residue was purified by flash column chromatography to give the titled compound(crude), which was further purified to give the titled Compound A99a (101 mg) and Compound A99b (109 mg) by Prep-chiral HPLC. The chiral separation conditions are shown below.

| Column | IE |
|---|---|
| Column Size | 20.0 mm × 250 mm, 5 um |
| Mobile Phase A | HEXANE 40% |
| Mobile Phase B | ETOH(0.2% 2M NH3MEOH) 60% |
| Run Time | 12 min |
| Flow Rate | 18 mL/min |
| Wave Length | UV 210 nm and 310 nm |
| Temperature | RT |
| Prep-HPLC Equipment | Prep-HPLC-Gilson |

Compound A99a (the earlier peak): ¹H NMR (400 MHz, CD₃OD) δ 9.34 (s, 1H), 8.43 (d, J=8.5 Hz, 1H), 7.92 (s, 1H), 7.80 (d, J=8.5 Hz, 1H), 5.56 (s, 1H), 5.45 (s, 2H), 4.81-4.23 (m, 2H), 4.11 (q, J=6.5 Hz, 1H), 3.43 (s, 3H), 3.35 (d, J=10.5 Hz, 1H), 3.09-2.93 (m, 2H), 2.39 (d, J=8.6 Hz, 1H), 2.20-1.77 (m, 2H), 1.72-1.54 (m, 2H), 1.45 (d, J=6.6 Hz, 3H), 0.95 (t, J=7.5 Hz, 3H), 0.71 (t, J=7.4 Hz, 3H) ppm. MS: M/e 491 (M+1)⁺.

Compound A99b (the later peak): ¹H NMR (400 MHz, CD₃OD) δ 9.34 (s, 1H), 8.41 (d, J=8.5 Hz, 1H), 7.92 (s, 1H), 7.85 (d, J=8.5 Hz, 1H), 5.55 (s, 1H), 5.47 (s, 2H), 4.82-4.03 (m, 2H), 3.93 (q, J=6.5 Hz, 1H), 3.56 (d, J=12.7 Hz, 1H), 3.43 (s, 3H), 3.18 (d, J=9.6 Hz, 1H), 2.79 (dd, J=12.1, 3.4 Hz, 1H), 2.27 (d, J=12.2 Hz, 1H), 2.05-1.87 (m, 1H), 1.80-1.63 (m, 2H), 1.61-1.50 (m, 1H), 1.42 (d, J=6.6 Hz, 3H), 1.04 (t, J=7.3 Hz, 3H), 0.62 (t, J=7.4 Hz, 3H) ppm. MS: M/e 491 (M+1)⁺.

### Compound A100: 2-(7-((2S,5R)-5-ethyl-2-methyl-4-(1-(thiazolo[5,4-b]pyridin-5-yl)ethyl)piperazin-1-yl)-4-methyl-5-oxo-4,5-dihydro-2H-pyrazolo[4,3-b]pyridin-2-yl)acetonitrile

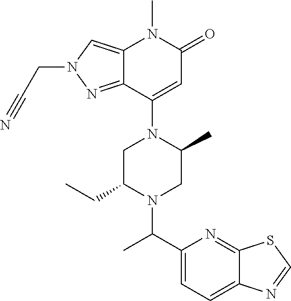 [SMILES: CC[C@@H]1CN(c2cc(=O)n(C)c3cn(CC#N)nc23)[C@@H](C)CN1C(C)c1ccc2ncsc2n1]

To a solution of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (150 mg, 0.47 mmol) in CHCN (8 mL) and was added 1-(thiazolo[5,4-b]pyridin-5-yl)ethan-1-ol (171 mg, 0.95 mmol), (cyanomethyl)trimethylphosphonium iodide (348 mg, 1.43 mmol) and DIPEA(0.61 g, 4.7 mmol).The resulting mixture was stirred at 105° C. overnight. The reaction solvent was removed under reduced pressure. The resulting residue was purified by flash column chromatography to give the titled compound(crude), which was further purified to give the titled Compound A100a (35 mg) and Compound A100b (35 mg) by Prep-chiral HPLC. The chiral separation conditions are shown below.

| Column | IE |
|---|---|
| Column Size | 20.0 mm × 250 mm, 5 um |
| Mobile Phase A | HEXANE 40% |
| Mobile Phase B | ETOH(0.2% 2M NH₃MEOH) 60% |
| Run Time | 13 min |
| Flow Rate | 18 mL/min |
| Wave Length | UV 210 nm and 310 nm |
| Temperature | RT |
| Prep-HPLC Equipment | Prep-HPLC-Gilson |

Compound A100a (the earlier peak): ¹H NMR (400 MHz, CD₃OD) 9.34 (s, 1H), 8.43 (d, J=8.5 Hz, 1H), 7.92 (s, 1H), 7.81 (d, J=8.5 Hz, 1H), 5.58 (s, 1H), 5.45 (s, 2H), 4.81-4.45 (m, 2H), 4.12 (q, J=6.4 Hz, 1H), 3.43 (s, 3H), 3.41-3.33 (m, 1H), 3.06 (dd, J=11.9, 4.0 Hz, 1H), 2.87 (d, J=9.5 Hz, 1H), 2,42 (s, 1H), 1.62 (dd, J=14.7, 7.3 Hz, 2H), 1.45 (d, J=5.6 Hz, 3H), 1.40 (d, J=6.4 Hz, 3H), 0.74 (d, J=7.4 Hz, 3H) ppm. MS: M/e 477 (M+1)⁺.

Compound A100b (the later peak): ¹H NMR (400 MHz, CD₃OD) δ 9.33 (s, 1H), 8.41 (d, J=8.5 Hz, 1H), 7.93 (s, 1H), 7.88 (d, J=8.6 Hz, 1H), 5.57 (s, 1H), 5.47 (s, 2H), 5.01 (s, 1H), 4.44 (s, 1H), 3.94 (q, J=6.8 Hz, 1H), 3.59 (d, J=12.9 Hz, 1H), 3.43 (s, 3H), 3.19 (d, J=10.4 Hz, 1H), 2.87 (dd, J=12.0, 4.1 Hz, 1H), 2.18 (d, J=12,4 Hz, 1H), 1.80-1.45 (m, 2H), 1.43 (d, J=6.7 Hz, 3H), 1,24 (d, J=6.5 Hz, 3H), 1.07 (t, J=7.4 Hz, 3H) ppm. MS: M/e 477 (M+1)⁺.

### Compound A101: 2-(7-((2S,5R)-2,5-diethyl-4-(1-(4-fluorobenzo[d]thiazol-6-yl)ethyl)piperazin-1-yl)-4-methyl-5-oxo-4,5-dihydro-2H-pyrazolo[4,3-b]pyridin-2-yl)acetonitrile

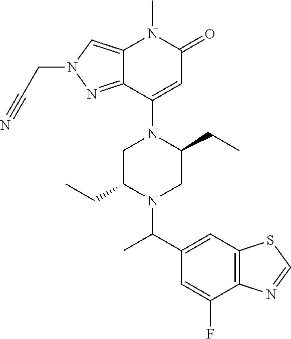 [SMILES: CC[C@H]1CN(C(C)c2cc(F)c3ncsc3c2)[C@H](CC)CN1c1cc(=O)n(C)c2cn(CC#N)nc12]

### Step A: 6-bromo-4-fluorobenzo[d]thiazol-2-amine

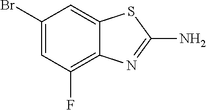 [SMILES: Nc1nc2c(F)cc(Br)cc2s1]

To a stirred solution of 4-bromo-2-fluoroaniline (1 g, 5.26 mmol) and KSCN (1.02 g, 10.52 mmol) in AcOH (10 ml) was added Br₂ (0.84 g, 5.25 mmol) at 0° C. The reaction was stirred at RT overnight. The solution was concentrated under reduced pressure. The resulting residue was diluted with EA (20 ml), washed with aq. NaHCO₃ (10 ml×2) and brine (10 ml), dried over Na₂SO₄ and concentrated to dryness. The resulting residue was purified by flash column chromatography with 0-40% EA in PE to give the titled compound (386 mg, 30%). MS: M/e 247 (M+1)⁺.

### Step B: 6-bromo-4-fluorobenzo[d]thiazole

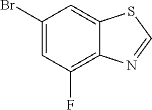 [SMILES: Fc1cc(Br)cc2scnc12]

A solution of 6-bromo-4-fluorobenzo[d]thiazol-2-amine (386 mg, 1.57 mmol) and tert-butyl nitrite (404 mg, 3.92 mmol) in THF (8 ml) was stirred at 70° C. for 3 hours. The solution was quenched with water (10 ml) and extracted with EA (10 ml×2). The organic layer was dried over Na₂SO₄ and concentrated to dryness. The resulting residue was purified by flash column chromatography with 0-15% EA in PE to give the titled compound (290 mg, 80%). MS: M/e 232,234 (M+1)⁺.

### Step C: 1-(4-fluorobenzo[d]thiazol-6-yl)ethan-1-one

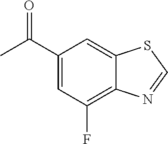

A solution of 6-bromo-4-fluorobenzo[d]thiazole (290 mg, 1,25 mmol), ethyl tributylstannanecarboxylate (677 mg, 1.88 mmol) and Pd(PPh₃)₂Cl₂ (44 mg, 0.063 mmol) in toluene(10 ml) was stirred at 100° C. overnight. The solution was cooled to RT. HCl/1,4-dioxane (4M, 2 ml) was added and stirred at RT for 10 min. The solution was diluted with EtOAc (15 ml), washed with aq. Na₂CO₃ (10 ml×2), dried over Na₂SO₄ and concentrated to dryness. The resulting residue was purified by flash column chromatography with 0-40% EA in PE to give the titled compound (190 mg, 78%). MS: M/e 196 (M+1)⁺.

### Step D: 1-(4-fluorobenzo[d]thiazol-6-yl)ethan-1-ol

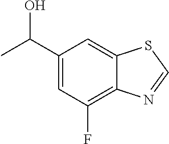

A solution of 1-(4-fluorobenzo[d]thiazol-6-yl)ethan-1-one (190 mg, 0.97 mmol) and NaBH₄ (37 mg, 0.97 mmol) in MeOH (3 ml) was stirred at RT for 15 min. The solution was diluted with EtOAc (10 ml), washed with brine (10 ml×2), dried over Na₂SO₄ and concentrated to dryness to give the titled compound (190 mg, 99%), which was used directly for the next step without further purification. MS: We 198 (M+1)⁺.

### Step E: 2-(7-((2S,5R)-2,5-diethyl-4-(1-(4-fluorobenzo[d]thiazol-6-yl)ethyl)piperazin-1-yl)-4-methyl-5-oxo-4,5-dihydro-2H-pyrazolo[4,3-b]pyridin-2-yl)acetonitrile

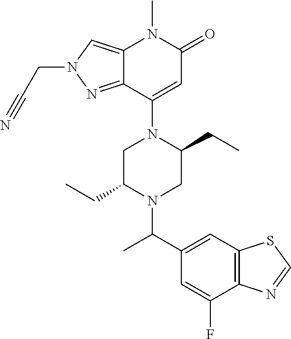 [SMILES: CC[C@H]1CN(C(C)c2cc(F)c3ncsc3c2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol), 1-(4-fluorobenzo[d]thiazol-6-yl)ethan-1-ol (60 mg, 0.30 mmol), (cyanomethyl)trimethylphosphonium iodide (111 mg, 0.46 mmol) and DIPEA (197 mg, 1.53 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A101, which was further separated into Compound A101a (14 mg) and Compound A101b (14 mg) by Prep-HPLC (Method A).

Compound A101a (the earlier peak): ¹H NMR (400 MHz, DMSO-d₆) δ 9.36 (s, 1H), 8.30 (s, 0.3H), 7.96 (d, J=13.1 Hz, 2H), 7.37 (d, J=11.4 Hz, 1H), 5.57 (s, 2H), 5.36 (s, 1H), 3.87 (s, 1H), 3.32 (s, 2H), 3.23 (s, 3H), 3.14 (d, J=12.1 Hz, 1H), 2.88 (d, J=12.6 Hz, 1H), 2.78 (d, J=12.6 Hz, 1H), 2.29 (s, 1H), 2.00 (s, 1H), 1.62 (s, 1H), 1.45 (s, 2H), 1.30 (s, 3H), 0.83 (s, 3H), 0.58 (s, 3H) ppm. MS: M/e 508 (M+1)⁺.

Compound A101b (the later peak): ¹H NMR (400 MHz, DMSO-d₆) δ 9.35 (s, 1H), 8.23 (s, 0.16H), 7.95 (s, 2H), 7.39 (d, J=11.6 Hz, 1H), 5.57 (s, 2H), 5.35 (s, 1H), 3.72 (s, 1H), 3.44-3.31 (m, 3H), 3.23 (s, 3H), 3.02 (d, J=7.9 Hz, 1H), 2,52 (d, J=10.9 Hz, 1H), 2.19 (d, J=11.9 Hz, 1H), 1.88 (s, 1H), 1.56 (s, 1H), 1.43 (s, 2H), 1,26 (s, 3H), 0.92 (s, 3H), 0.47 (s, 3H) ppm. MS: M/e 508 (M+1)⁺.

### Compound A102: 2-(7-((2S,5R)-2,5-diethyl-4-(1-(7-fluorobenzo[d]thiazol-6-yl)ethyl)piperazin-1-yl)-4-methyl-5-oxo-4,5-dihydro-2H-pyrazolo[4,3-b]pyridin-2-yl)acetonitrile

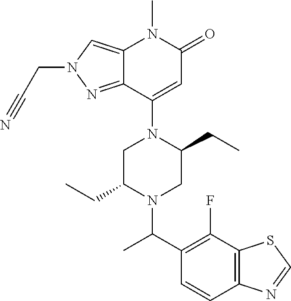 [SMILES: CC[C@H]1CN(C(C)c2ccc3ncsc3c2F)[C@H](CC)CN1c1cc(=O)n(C)c2cn(CC#N)nc12]

### Step A: N-((4-bromo-2.3-difluorophenyl)carbamothioyl)benzamide

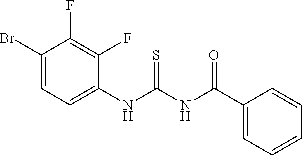 [SMILES: O=C(NC(=S)Nc1ccc(Br)c(F)c1F)c1ccccc1]

A solution of 4-bromo-2,3-difluoroaniline (2 g, 9.62 mmol) and benzoyl isothiocyanate (1.65 g, 10.12 mmol) in acetone (30 ml) was stirred at RT overnight. The reaction was filtered. The filter cake was dried to give the titled compound (2,53 g, 71%). MS: M/e 371 (M+1)⁺.

### Step B: 1-(4-bromo-23-difluorophenyl)thiourea

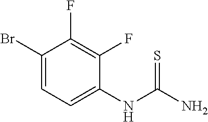 [SMILES: NC(=S)Nc1ccc(Br)c(F)c1F]

A solution of N-((4-bromo-2,3-difluorophenyl)carbamothioyl)benzamide (2,53 g, 6.84 mmol) and NaOH (816 mg, 20.4 mmol) in MeOH (25 ml) and water (5 ml) was stirred at 70° C. for 30 min. The reaction was concentrated under reduced pressure. The resulting residue was filtered. The filter cake was dried to give the titled compound (0.83 g, 46%). MS: We 267,269 (M+1)⁺.

### Step C: 6-bromo-7-fluorobenzoldithiazol-2-amine

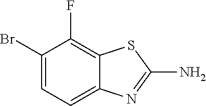 [SMILES: Nc1nc2ccc(Br)c(F)c2s1]

A solution of 1-(4-bromo-2,3-difluorophenyl)thiourea (830 mg, 3.12 mmol) and NaH (60%, 188 mg, 4.70 mmol) in NMP (10 ml) was stirred at 100° C. for 1 h. The reaction was quenched with water (20 ml) and extracted with EA (10 ml×2). The organic layer was dried over Na₂SO₄ and concentrated to dryness. The resulting residue was purified by flash column chromatography with 10-40% EA in PE to give the titled compound (760 mg, crude), which was used directly for the next step without further purification. MS: We 247,249 (M+1)⁺.

### Step D: 6-bromo-7-fluorobenzo[d]thiazole

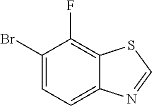 [SMILES: Fc1c(Br)ccc2ncsc12]

A solution of 6-bromo-7-fluorobenzo[d]thiazol-2-amine (760 mg, crude, 3.09 mmol) and tert-butyl nitrite (800 mg, 7.77 mmol) in THF (10 ml) was stirred at 70° C. for 2 h. The solution was quenched with water (10 ml) and extracted with EA (10 ml×2). The organic layer was dried over Na₂SO₄ and concentrated to dryness. The resulting residue was purified by flash column chromatography with 0-15% EA in PE to give the titled compound (390 mg). MS: M/e 232,234 (M+1)⁺.

### Step E: 1-(7-fluorobenzo[d]thiazol-6-yl)ethan-1-one

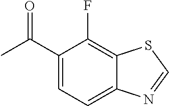

A solution of 6-bromo-7-fluorobenzo[d]thiazole (390 mg, 1.68 mmol), ethyl tributylstannanecarboxylate (910 mg, 2,52 mmol) and Pd(PPh₃)₂Cl₂ (59 mg, 0.084 mmol) in toluene(10 ml) was stirred at 100° C. overnight. The solution was cooled to RT. HCl/1,4-dioxane (4M, 2 ml) was added and stirred at RT for 10 min. The solution was diluted with EtOAc (15 ml), washed with aq. Na₂CO₃ (10 ml×2), dried over Na₂SO₄ and concentrated to dryness. The resulting residue was purified by flash column chromatography with 0-25% EA in PE to give the titled compound (264 mg, 81%). MS: We 196 (M+1)⁺.

### Step F: 1-(7-fluorobenzo[d]thiazol-6-yl)ethan-1-ol

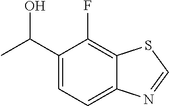

A solution of 1-(7-fluorobenzo[d]thiazol-6-yl)ethan-1-one (264 mg, 1.35 mmol) and NaBH₄ (51.4 mg, 1.35 mmol) in MeOH (4 ml) was stirred at RT for 15 min. The solution was diluted with EtOAc (10 ml), washed with brine (10 ml×2), dried over Na₂SO₄ and concentrated to dryness to give the titled compound (264 mg, 99%), which was used directly for the next step without further purification. MS: M/e 198 (M+1)⁺.

### Step G: 2-(7-((2S,5R)-2,5-diethyl-4-(1-(7-fluorobenzo[d]thiazol-6-yl)ethyl)piperazin-1-yl)-4-methyl-5-oxo-4,5-dihydro-2H-pyrazolo[4,3-b]pyridin-2-yl)acetonitrile

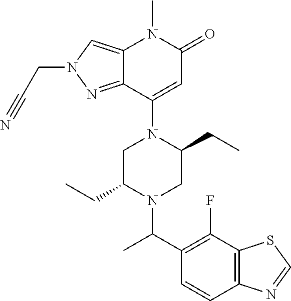 [SMILES: CC[C@H]1CN(C(C)c2ccc3ncsc3c2F)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (200 mg, 0.61 mmol), 1-(7-fluorobenzo[d]thiazol-6-yl)ethan-1-ol (200 mg, 1.02 mmol), (cyanomethyl)trimethylphosphonium iodide (444.5 mg, 1.83 mmol) and DIPEA (787 mg, 6.10 mmol) in CH₃CN (6 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A102, which was further separated into Compound A102a (64 mg) and Compound A102b (76 mg) by Prep-HPLC (Method A).

Compound A102a (the earlier peak): ¹H NMR (400 MHz, CD₃OD) δ 9.25 (s, 1H), 7.97-7.87 (m, 2H), 7.79-7.71 (m, 1H), 5.54 (s, 1H), 5.44 (s, 2H), 4.33 (s, 1H), 3.41 (s, 3H), 3.30 (s, 3H), 3.00 (d, J=11.2 Hz, 1H), 2.93 (d, J=11.2 Hz, 1H), 2.37 (s, 1H), 2.07 (s, 1H), 1.82 (s, 1H), 1.55 (s, 2H), 1.43 (d, J=5.9 Hz, 3H), 0.95 (t, J=7.9 Hz, 3H), 0.69 (t, J=7.9 Hz, 3H) ppm. MS: M/e 508 (M+1)⁺.

Compound A102b (the later peak): ¹H NMR (400 MHz, CD₃OD) δ 9.24 (s, 1H), 7.90 (d, J=8.6 Hz, 2H), 7.84 (t, J=7.5 Hz, 1H), 5.54 (s, 1H), 5.45 (s, 2H), 4.19 (q, J=6.0 Hz, 1H), 3.51 (d, J=13.2 Hz, 1H), 3.41 (s, 3H), 3.30 (s, 2H), 3.18 (d, J=9.5 Hz, 1H), 2.72 (d, J=11.3 Hz, 1H), 2.32 (d, J=11.9 Hz, 1H), 1.97 (s, 1H), 1.75-1.52 (m, 3H), 1.39 (d, J=5.7 Hz, 3H), 1.03 (t, J=6.9 Hz, 3H), 0.58 (t, J=6.7 Hz, 3H) ppm. MS: M/e 508 (M+1)⁺.

### Compound A103: 2-(7-((2S,5R)-2,5-diethyl-4-(1-(5-fluorobenzo[d]thiazol-6-yl)ethyl)piperazin-1-yl)-4-methyl-5-oxo-4,5-dihydro-2H-pyrazolo[4,3-b]pyridin-2-yl)acetonitrile

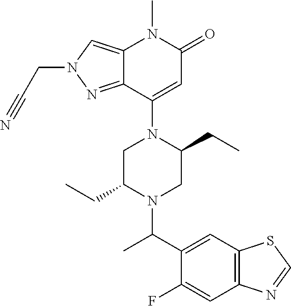 [SMILES: CC[C@H]1CN(C(C)c2cc3scnc3cc2F)[C@H](CC)CN1c1cc(=O)n(C)c2cn(CC#N)nc12]

### Step A: 6-bromo-5-fluorobenzo[d]thiazol-2-amine

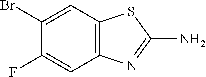 [SMILES: Nc1nc2cc(F)c(Br)cc2s1]

A mixture of 4-bromo-3-fluoroaniline (1 g, 5.3 mmol) and KSCN (760 mg, 7.9 mmol) in AcOH (3 mL) was added Br₂ (0.85 g, 5.3 mmol) at 0° C., then warmed at room temperature for 2 hours. The solution was concentrated and diluted with water. The solution was concentrated and adjusted to pH=8-9 with saturated NaHCO₃ solution, extracted with ethyl acetate. The combined organic layers were purified by flash column chromatography to give the titled compound (800 mg, 61%). MS: M/e 247 (M+1)⁺

### Step B: 6-bromo-5-fluorobenzo[d]thiazole

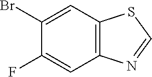 [SMILES: Fc1cc2ncsc2cc1Br]

A mixture of 6-bromo-5-fluorobenzo[d]thiazol-2-amine (800 mg, 3.2 mmol), tBuONO (474 mg, 14.8 mmol) in THF (10 mL) was stirred at 70° C. for 2 hours. The solution was concentrated to dryness. The resulting residue was purified by flash column chromatography to give the titled compound (600 mg, 80%). MS: We 232 (M+1)⁺.

### Step C: 1-(5-fluorobenzo[d]thiazol-6-yl)ethan-1-one

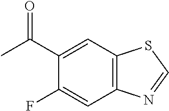

A mixture of 6-bromo-5-fluorobenzo[d]thiazole (300 mg, 1,25 mmol), tributyl(1-ethoxyvinyl)stannane (0.7 g, 1.9 mmol) and Pd(PPh₃)₂Cl₂ (90 mg, 0.12 mmol) in DMF (5 mL) was stirred at 100° C. under N₂ for 16 hours. The solution was diluted with ethyl acetate and washed with water. The organic layer was dried and added with HCl (3 mL, 3 M in ethyl acetate) in drops and the mixture was stirred at room temperature for 3 hours. The reaction was concentrated and adjusted to pH=8-9 with saturated NaHCO₃ solution, extracted with ethyl acetate. The combined organic layers were washed with brine, dried over Na₂SO₄ and the resulting residue was purified by flash column chromatography to give the titled compound (200 mg, 79%). MS: M/e 196 (M+1)⁺

### Step D: 1-(5-fluorobenzo[d]thiazol-6-yl)ethan-1-ol

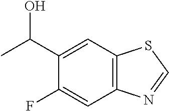

To a solution of 1-(5-fluorobenzo[d]thiazol-6-yl)ethan-1-one (200 mg, 1.02 mmol) in MeOH (3 mL) was added NaBH₄ (38 mg, 1.02 mmol) at room temperature and the resulting mixture was stirred at room temperature for 5 min. The reaction mixture was diluted with DCM and washed with water, dried over Na₂SO₄ and concentrated to give the titled compound (190 mg). MS: M/e 198 (M+1)⁺.

### Step E: 2-(7-((2S,5R)-2,5-diethyl-4-(1-(5-fluorobenzo[d]thiazol-6-yl)ethyl)piperazin-1-yl-4-methyl-5-oxo-4,5-dihydro-2H-pyrazolo[4,3-b]pyridin-2-yl)acetonitrile

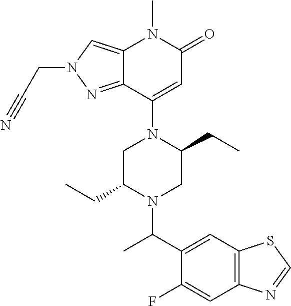 [SMILES: CC[C@H]1CN(C(C)c2cc3scnc3cc2F)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (40 mg, 0.12 mmol) in CH₃CN (3 mL) and was added 1-(5-fluorobenzo[d]thiazol-6-yl)ethan-1-ol (72 mg, 0.36 mmol), (cyanomethyl)trimethylphosphonium iodide (88 mg, 0.36 mmol) and DIPEA(157 mg, 1,2 mmol).The resulting mixture was stirred at 105° C. overnight. The reaction solvent was removed under reduced pressure. The resulting residue was purified by flash column chromatography to give the titled compound(crude), which was further purified to give the titled Compound A103a (8 mg) and Compound 103b (8 mg) by Prep-HPLC (Method A).

Compound A103a (the earlier peak): ¹H NMR (400 MHz, CD₃OD) δ 9.27 (s, 1H), 8.23 (d, J=6.6 Hz, 1H), 7.90 (s, 1H), 7.76 (d, J=10.8 Hz, 1H), 5.54 (s, 1H), 5.44 (s, 2H), 4.85 (s, 1H), 4.32 (q, J=6.5 Hz, 1H), 3.41 (s, 3H), 3.31 (d, 0.1=10.7 Hz, 2H), 3.06-2.87 (m, 2H), 2,46 (s, 1H), 2.14-1.99 (m, 1H), 1.91-1.74 (m, 1H), 1.55 (dd, J=14.4, 7.3 Hz, 2H), 1.43 (d, J=6.6 Hz, 3H), 0.93 (t, J=7.5 Hz, 3H), 0.73 (t, J=7.4 Hz, 3H) ppm. MS: M/e 508 (M+1)⁺.

Compound A103b (the later peak): ¹H NMR (400 MHz, CD₃OD) δ 9.25 (s, 1H), 8.33 (d, J=6.7 Hz, 1H), 7.91 (s, 1H), 7.74 (d, J=10.8 Hz, 1H), 5.54 (s, 1H), 5.45 (s, 2H), 4.85 (s, 1H), 4.14 (d, J=6.7 Hz, 1H), 3.53 (d, J=14.7 Hz, 1H), 3.42 (s, 3H), 3.27-3.09 (m, 2H), 2.72 (d, J=11.9 Hz, 1H), 2.38 (d, J=12.1 Hz, 1H), 2.13-1.53 (m, 4H), 1.38 (d, J=6.6 Hz, 3H), 1.03 (t, J=7.4 Hz, 3H), 0.58 (t, J=7.4 Hz, 3H) ppm. MS: M/e 508 (M+1)⁺.

### Compound A104: 2-(7-((2S,5R)-5-ethyl-4-(1-(7-fluoro-2-methylbenzo[d]thiazol-6-yl)ethyl)-2-methylpiperazin-1-yl)-4-methyl-5-oxo-4,5-dihydro-2H-pyrazolo[4,3-b]pyridin-2-yl)acetonitrile

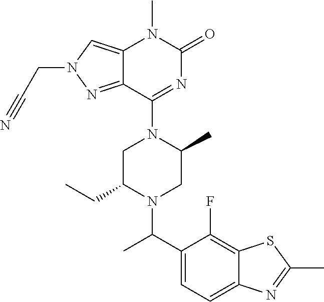 [SMILES: CC[C@@H]1CN(c2nc(=O)n(C)c3cn(CC#N)nc23)[C@@H](C)CN1C(C)c1ccc2nc(C)sc2c1F]

### Step A: N-(4-bromo-2.3-difluorophenyl)acetamide

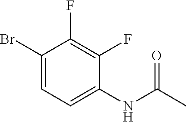

A solution of 4-bromo-2,3-difluoroaniline (2.7 g, 12.98 mmol) and acetic anhydride (2.7 ml) in EA (30 ml) was stirred at 50° C. for 1.5 hrs. The solution was washed with aq. Na₂CO₃ (20 ml×2), dried over Na₂SO₄ and concentrated to dryness to give the titled compound (3.2 g, 99%). MS: M/e 250 (M+1)⁺.

### Step B: N-(4-bromo-23-difluorophenyl)ethanethioamide

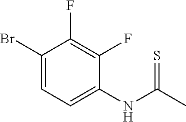

A solution of N-(4-bromo-2,3-difluorophenyl)acetamide (3 g, 12.05 mmol) and Lawesson's Reagent (2,43 g, 6.01 mmol) in toluene (30 ml) was stirred at 110° C. for 1.5 hrs. The solution was diluted with EtOAc (30 ml), washed with brine (10 ml×2), dried over Na₂SO₄ and concentrated to dryness. The resulting residue was purified by flash column chromatography with 0-15% EA in PE to give the titled compound (3 g, 94%). MS: M/e 266 (M+1)⁺.

### Step C: 6-bromo-7-fluoro-2-methylbenzo[d]thiazole

A solution of N-(4-bromo-2,3-difluorophenyl)ethanethioamide (3 g, 11.32 mmol) and Cs₂CO₃ (7.4 g, 22.70 mmol) in toluene(30 ml) was stirred at 110° C. for 1.5 hrs. The solution was diluted with EtOAc (30 ml), washed with brine (10 ml×2), dried over Na₂SO₄ and concentrated to dryness. The resulting residue was purified by flash column chromatography with 0-10% EA in PE to give the titled compound (2.1 g, 76%). MS: M/e 246 (M+1)⁺.

### Step D: 1-(7-fluoro-2-methylbenzo[d]thiazol-6-yl)ethan-1-one

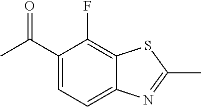

A solution of 6-bromo-7-fluoro-2-methylbenzo[d]thiazole (2.1 g, 8.57 mmol), ethyl tributylstannanecarboxylate (4.64 g, 12.85 mmol) and Pd(PPh₃)₂Cl₂ (300 mg, 0.43 mmol) in toluene(20 ml) was stirred at 100° C. overnight. The solution was cooled to RT. HCl/1,4-dioxane (4M, 2 ml) was added and stirred at RT for 10 min. The solution was diluted with EtOAc (20 ml), washed with aq. Na₂CO₃ (10 ml×2), dried over Na₂SO₄ and concentrated to dryness. The resulting residue was purified by flash column chromatography with 0-20% EA in PE to give the titled compound (1.7 g, 94%). MS: M/e 210 (M+1)⁺.

### Step E: 147-fluoro-2-methylbenzo[d]thiazol-6-yl)ethan-1-ol

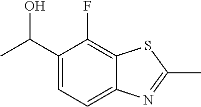

A solution of 1-(7-fluoro-2-methylbenzo[d]thiazol-6-yl)ethan-1-one (1.7 g, 8.13 mmol) and NaBH₄ (309 mg, 8.13 mmol) in MeOH (15 ml) was stirred at RT for 10 min. The solution was diluted with EtOAc (30 ml), washed with brine (10 ml×2), dried over Na₂SO₄ and concentrated to dryness to give the titled compound (1.7 g, ⁹⁹⁰/), which was used directly for the next step without further purification. MS: M/e 212 (M+1)⁺.

### Step F: 2-(7-((2S,5R)-5-ethyl-4-(1-(7-fluoro-2-methylbenzo[d]thiazol-6-yl)ethyl)-2-methylpiperazin-1-yl)-4-methyl-5-oxo-4,5-dihydro-2H-pyrazolo[4,3-b]pyridin-2-yl)acetonitrile

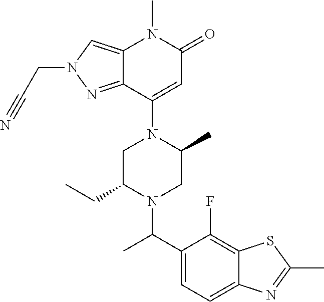 [SMILES: CC[C@@H]1CN(c2cc(=O)n(C)c3cn(CC#N)nc23)[C@@H](C)CN1C(C)c1ccc2nc(C)sc2c1F]

A solution of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (50 mg, 0.16 mmol), 1-(7-fluoro-2-methylbenzo[d]thiazol-6-yl)ethan-1-ol (67.2 mg, 0.32 mmol), (cyanomethyl)trimethylphosphonium iodide (116 mg, 0.48 mmol) and DIPEA (205 mg, 1.59 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography to give the titled Compound A104, which was further separated into Compound A104a (14 mg) and Compound A104b (10 mg) by Prep-HPLC (Method A).

Compound A104a (the earlier peak): ¹H NMR (400 MHz, CD₃CD) 6 7.92 (s, 1H), 7.76 (d, J=8.5 Hz, 1H), 7.70 (t, J=6.8 Hz, 1H), 5.57 (s, 1H), 5.45 (s, 2H), 4.33 (q, J=6.7 Hz, 1H), 3.43 (s, 3H), 3.32 (s, 2H), 3.29-3.23 (m, 1H), 3.02 (dd, J=11.99, 4.00 Hz, 1H), 2.89-2.80 (m, 4H), 2,41 (s, 1H), 1.65-1.51 (m, 2H), 1.43 (d, J=6.5 Hz, 3H), 1.39 (d, J=6.4 Hz, 3H), 0.76 (t, J=7.3 Hz, 3H) ppm. MS: M/e 508 (M+1)⁺.

Compound A104b (the later peak): ¹H NMR (400 MHz, CD₃CD) 6 7.93 (s, 1H), 7.82-7.75 (m, 1H), 7.72 (d, J=8.5 Hz, 1H), 5.57 (s, 1H), 5.47 (s, 2H), 4.18 (q, J=6.5 Hz, 1H), 3.54 (d, J=12.7 Hz, 1H), 3.43 (s, 3H), 3.31 (s, 2H), 3.18 (d, J=10.2 Hz, 1H), 2.89-2.77 (m, 4H), 2.22 (d, J=11.6 Hz, 1H), 1.80-1.66 (m, 1H), 1.64-1.51 (m, 1H), 1.40 (d, J=6.6 Hz, 3H), 1,23 (d, J=6.5 Hz, 3H), 1.07 (t, J=7.3 Hz, 3H) ppm. MS: M/e 508 (M+1)⁺.

### Compound A105: 2-(7-((2S,5R)-4-(1-(benzo[dloxazol-5-yl)ethyl)-2,5-diethylpiperazin-1-yl)-4-methyl-5-oxo-4,5-dihydro-2H-pyrazolo[4,3-b]pyridin-2-yl)acetonitrile

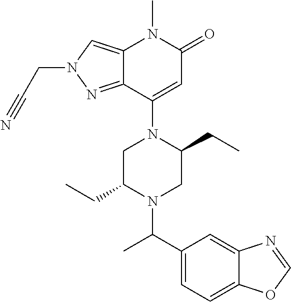 [SMILES: CC[C@H]1CN(C(C)c2ccc3ocnc3c2)[C@H](CC)CN1c1cc(=O)n(C)c2cn(CC#N)nc12]

### Step A: 1-(benzo[d]oxazol-5-yl)ethan-1-one

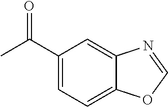

A mixture of 5-bromobenzo[d]oxazole (300 mg, 1.52 mmol), tributyl(1-ethoxyvinyl)stannane (827 mg, 2.28 mmol) and Pd(PPh₃)₂Cl₂ (106 mg, 0.15 mmol) in DMF (5 mL) was stirred at 100° C. under N₂ for 16 hours. The solution was diluted with ethyl acetate and washed with water. The organic layer was dried and purified by flash column chromatography to give the titled compound (130 mg, 53%). MS: M/e 162 (M+1)⁺

### Step B: 14benzo[d]oxazol-5-yl)ethan-1-ol

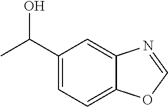

To a solution of 1-(benzo[d]oxazol-5-yl)ethan-1-one (70 mg, 0.43 mmol) in MeOH (3 mL) was added NaBH₄ (11 mg, 0.3 mmol) at room temperature and the resulting mixture was stirred at room temperature for 5 min. The reaction mixture was diluted with DCM and washed with water, dried over Na₂SO₄ and concentrated to give the titled compound (70 mg). MS: M/e 164 (M+1)⁺.

### Step C: 2-(7-((2S,5R)-4-(1-(benzo[dloxazol-5-yl)ethyl)-2,5-diethylpiperazin-1-yl)-4-methyl-5-oxo-4,5-dihydro-2H-pyrazolo[4,3-b]pyridin-2-yl)acetonitrile

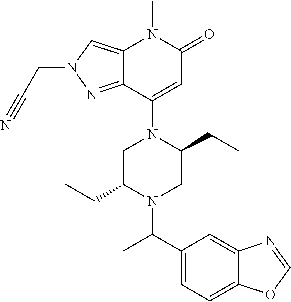 [SMILES: CC[C@H]1CN(C(C)c2ccc3ocnc3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(benzo[d]oxazol-5-yl)ethan-1-ol (70 mg, 0.42 mmol), (cyanomethyl)trimethylphosphonium iodide (111 mg, 0.45 mmol) and DIPEA(197 mg, 1.5 mmol).The resulting mixture was stirred at 105° C. overnight. The reaction solvent was removed under reduced pressure. The resulting residue was purified by flash column chromatography to give the titled compound(crude), which was further purified to give the titled compound (7 mg) by Prep-HPLC (MethodB). ¹H NMR (400 MHz, DMSO-d₆) δ 8.70 (d, J=6.6 Hz, 1H), 7.96 (d, J=1.6 Hz, 1H), 7.80-7.65 (m, 2H), 7.45 (t, J=8.3 Hz, 1H), 5.59 (s, 2H), 5.37 (s, 1H), 3.78 (dd, J=55.8, 6.3 Hz, 1H), 3.25 (s, 4H), 3.12-2.62 (m, 3H), 2.36-2.12 (m, 2H), 1.95 (d, J=85.9 Hz, 1H), 1.70-1.38 (m, 3H), 1,28 (dd, J=14.5, 6.4 Hz, 3H), 0.92 (dd, J=23.6, 16.1 Hz, 3H), 0.54 (dd, J=29.7, 22.2 Hz, 3H) ppm. MS: M/e 474 (M+1)⁺.

### Compound A106: 2-(7-((2S,5R)-4-(1-(benzo[c][1,2,5]thiadiazol-5-yl)ethyl)-2,5-diethylpiperazin-1-yl)-4-methyl-5-oxo-4,5-dihydro-2H-pyrazolo[4,3-b]pyridin-2-yl)acetonitrile

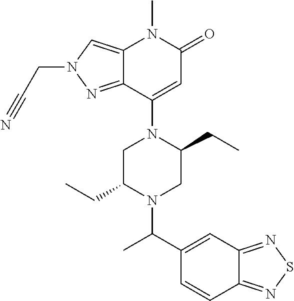 [SMILES: CC[C@H]1CN(C(C)c2ccc3nsnc3c2)[C@H](CC)CN1c1cc(=O)n(C)c2cn(CC#N)nc12]

### Step A: 1-(benzolcIll.2,5lthiadiazol-5-yl)ethan-1-one

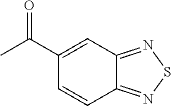

A mixture of 5-bromobenzo[c][1,2,5]thiadiazole (645 mg, 3 mmol), tributyl(1-ethoxyvinyl)stannane (1.6 g, 4.5 mmol) and Pd(PPh₃)₂Cl₂ (210 mg, 0.3 mmol) in toluene (5 mL) was stirred at 100° C. under N₂ for 16 hours. To the resulting solution was added HCl (0.5 mL, con.) in drops and the mixture was stirred at room temperature for 30 minutes. The mixture was treated with saturated NaHCO₃ aq. (20 ml), extracted with EtOAc(10 ml×3), washed with brine (20 mL), dried and concentrated to dryness. The resulting residue was purified by flash column chromatography (EtOAc: PE=0-50% in 30 minutes) to give the titled compound (450 mg, 84%). MS: M/e 179 (M+1)⁺.

### Step B: 1-(benzo[c][1,2,5]thiadiazol-5-yl)ethan-1-ol

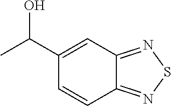

To a solution of 1-(benzo[c][1,2,5]thiadiazol-5-yl)ethan-1-one (178 mg, 1 mmol) in MeOH (3 mL) was added NaBH₄ (30 mg, 0.8 mmol) at room temperature and the mixture was stirred at room temperature for 30 minutes. The resulting mixture was treated with water (20 ml), extracted with DCM (10 mL×2). The combined organic layers were dried over Na₂SO₄ and concentrated to dryness. The resulting residue (150 mg, crude) was used in the next step directly. MS: M/e 181 (M+1)”.

### Step C: 2-(7-((2S,5R)-4-(1-(benzo[c][1,2,5]thiadiazol-5-yl)ethyl)-2,5-diethylpiperazin-1-yl-4-methyl-5-oxo-4,5-dihydro-2H-pyrazolo[4,3-b]pyridin-2-yl)acetonitrile

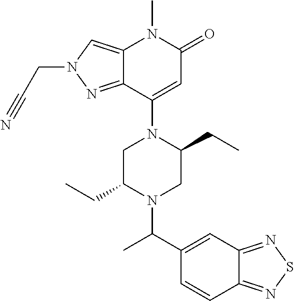 [SMILES: CC[C@H]1CN(C(C)c2ccc3nsnc3c2)[C@H](CC)CN1c1cc(=O)n(C)c2cn(CC#N)nc12]

A mixture of 1-(benzo[c][1,2,5]thiadiazol-5-yl)ethan-1-ol (72 mg, 0.4 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (65 mg, 0.2 mmol), (cyanomethyl)trimethylphosphonium iodide (193 mg, 0.8 mmol) and DIPEA (103 mg, 0.8 mmol) in MeCN (3 mL) was stirred at 100° C. overnight. The reaction mixture was diluted with EtOAc (20 mL), washed with water (10 mL×2), dried over Na₂SO₄ and concentrated to dryness. The resulting residue was purified by flash column chromatography (MeOH:DCM=0-10% in 30 minutes) to give the compound (−50 mg, crude) which was separated into Compound A106a (5 mg) and Compound A106b (5 mg) by Prep- HPLC (Method A).

Compound A106a (the earlier peak): ¹H NMR (500 MHz, DMSO-d₆) δ 8.09 (d, J=9.1 Hz, 1H), 7.99 (d, J=17.5 Hz, 2H), 7.83 (dd, J=9.1, 1.4 Hz, 1H), 5.60 (s, 2H), 5.40 (s, 1H), 3.99 (q, J=6.4 Hz, 1H), 3.27 (s, 5H), 3.16 (d, J=12.0 Hz, 1H), 2.96 (d, J=11.6 Hz, 1H), 2.82 (d, J=8.4 Hz, 1H), 2.35 (d, J=10.1 Hz, 1H), 2.05 (dt, J=15.4, 7.4 Hz, 1H), 1.71-1.45 (m, 3H), 1.37 (d, J=6.5 Hz, 3H), 0.87 (t, J=7.1 Hz, 3H), 0.62 (t, .=7.3 Hz, 3H) ppm. MS: M/e 491 (M+1)⁺.

Compound A106a (the later peak): ¹H NMR (500 MHz, DMSO-d₆) δ 8.07 (d, J=9.1 Hz, 1H), 8.00 (d, J=14.9 Hz, 2H), 7.84 (dd, J=9.1, 1.5 Hz, 1H), 5.61 (s, 2H), 5.40 (s, 1H), 3.84 (q, J=6.3 Hz, 1H), 3.41 (d, J=12.8 Hz, 1H), 3.27 (s, 5H), 3.10 (d, J=10.0 Hz, 1H), 2.63 (d, J=9.3 Hz, 1H), 2.27 (d, J=11.9 Hz, 1H), 1.86 (dt, J=15.8, 8.1 Hz, 1H), 1.67-1.46 (m, 3H), 1.34 (d, J=6.5 Hz, 3H), 0.97 (t, J=7.3 Hz, 3H), 0.50 (t, J=7.1 Hz, 3H) ppm. MS: M/e 491 (M+1)⁺.

### Compound A107: 2-(7-((2S,5R)-4-(1-(benzo[d]thiazol-5-yl)ethyl)-2,5-diethylpiperazin-1-yl)-4-methyl-5-oxo-4,5-dihydro-2H-pyrazolo[4,3-b]pyridin-2-yl)acetonitrile

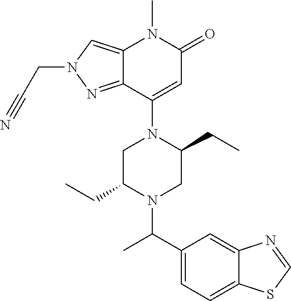 [SMILES: CC[C@H]1CN(C(C)c2ccc3scnc3c2)[C@H](CC)CN1c1cc(=O)n(C)c2cn(CC#N)nc12]

### Step A: 1-(benzo[d]thiazol-5-yl)ethan-1-one

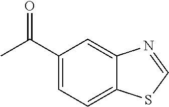

A mixture of 5-bromobenzo[d]thiazole (1.5 g, 7 mmol), tributyl(1-ethoxyvinyl)stannane (3.8 g, 10.5 mmol) and Pd(PPh₃)₂Cl₂ (493 mg, 0.7 mmol) in DMF (20 mL) was stirred at 100° C. under N₂ for 16 hours. The reaction mixture was diluted with HCl (5 mL, 3 M in ethyl acetate) in drops and the mixture was stirred at room temperature for 2 hours. The reaction was concentrated and adjusted to pH=8-9 with saturated NaHCO₃ solution, extracted with ethyl acetate. The combined organic layers were washed with brine, dried over Na₂SO₄ and the resulting residue was purified by flash column chromatography to give the titled compound (900 mg, 72%). MS: M/e 178 (M+1)⁺.

### Step B: 1-(benzo[d]thiazol-5-yl)ethan-1-ol

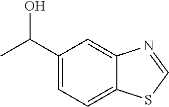

To a solution of 1-(benzo[d]thiazol-5-yl)ethan-1-one (900 mg, 5 mmol) in MeOH (10 mL) was added NaBH₄ (192 mg, 5 mmol) at room temperature and the resulting mixture was stirred at room temperature for 5 min. The reaction mixture was diluted with DCM and washed with water, dried over Na₂SO₄ and concentrated to give the titled compound (700 mg). MS: M/e 180 (M+1)⁺.

### Step C: 2-(7-((2S,5R)-4-(1-(benzo[d]thiazol-5-yl)ethyl)-2,5-diethylpiperazin-1-vib-4-methyl-5-oxo-4,5-dihydro-2H-pyrazolo[4,3-b]vridin-2-yl)acetonitrile

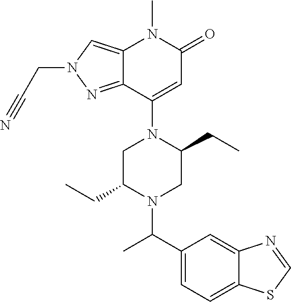 [SMILES: CC[C@H]1CN(C(C)c2ccc3scnc3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (300 mg, 0.91 mmol) in CH₃CN (8 mL) and was added 1-(benzo[d]thiazol-5-yl)ethan-1-ol (329 mg, 1.82 mmol), (cyanomethyl)trimethylphosphonium iodide (667 mg, 2.74 mmol) and DIPEA(1,2 g, 1.5 mmol).The resulting mixture was stirred at 105° C. overnight. The reaction solvent was removed under reduced pressure. The resulting residue was purified by flash column chromatography to give the titled compound(crude), which was further purified to give the titled Compound A107a (52 mg) and Con und A107b 66 m b Pre hira/HPLC. The chiral separation conditions are shown below.

| Column | Cellulose-C |
|---|---|
| Column Size | 20.0 mm × 250 mm, 5 um |
| Mobile Phase A | HEXANE 60% |
| Mobile Phase B | ETOH(0.2% 2M NH₃MEOH) 40% |
| Run Time | 12 min |
| Flow Rate | 18 mL/min |
| Wave Length | UV 220 nm and 310 nm |
| Temperature | RT |
| Prep-HPLC Equipment | Prep-HPLC-Gilson |

Compound A17a (the earlier peak):H NMR (400 MHz, CD₃OD) S9.25 (s, 1), 8.06 (d, J=8.5 Hz, 2H), 7.92 (s, 1H), 7.59 (d, J=8.3 Hz, 1H), 5.56 (s, 1H), 5.45 (s, 2H), 4.77-4.07 (m, 2H), 3.95 (q, J=6.2 Hz, 1H), 3.43 (s, 3H), 3.33 (s, 1H), 3.07 (d, J=12,4 Hz, 1H), 2.97-2.89 (m, 1H), 2,44 (d, J=8.7 Hz, 1H), 2.24-2.09 (m, 1H), 1.95-1.80 (m, 1H), 1.58 (d, J=7.4 Hz, 2H), 1.43 (d, J=6.4 Hz, 3H), 0.98 (t, J=7.3 Hz, 3H), 0.67 (t, J=7.4 Hz, 3H) ppm. MS: M/e 490 (M+1)”.

Compound A107b (the later peak): ¹H NMR (400 MHz, CD₃OD) δ 9.24 (s, 1H), 8.11 (s, 1H), 8.03 (d, J=8.3 Hz, 1H), 7.92 (s, 1H), 7.61 (d, J=8.4 Hz, 1H), 5.55 (s, 1H), 5.47 (s, 2H), 4.84-3.98 (m, 2H), 3.80 (q, J=6.4 Hz, 1H), 3.54 (d, J=13.5 Hz, 1H), 3.43 (s, 3H), 3.21 (d, J=9.0 Hz, 1H), 2.69 (d, J=8.6 Hz, 1H), 2.39 (d, J=12.2 Hz, 1H), 1.99 (d, J=6.7 Hz, 1H), 1.75-1.51 (m, 3H), 1.39 (d, J=6.5 Hz, 3H), 1.05 (t, J=7.3 Hz, 3H), 0.57 (t, J=7.4 Hz, 3H) ppm. MS: M/e 490 (M+1)⁺.

### Compound A108: 2-(7-((2S,5R)-2,5-diethyl-4-(1-(2-methylbenzo[d]thiazol-5-yl)ethyl)piperazin-1-yl)-4-methyl-5-oxo-4,5-dihydro-2H-pyrazolo[4,3-b]pyridin-2-yl)acetonitrile N N” r

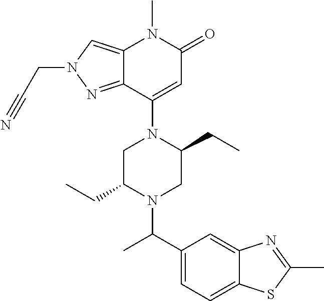 [SMILES: CC[C@H]1CN(C(C)c2ccc3sc(C)nc3c2)[C@H](CC)CN1c1cc(=O)n(C)c2cn(CC#N)nc12]

A mixture of 1-(2-methylbenzo[d]thiazol-5-yl)ethan-1-ol (29 mg, 0.15 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (48 mg, 0.2 mmol) and DIPEA (51 mg, 0.4 mmol) in MeCN (2 mL) was stirred at 100° C. overnight. The reaction mixture was diluted with EtOAc (10 mL), washed with brine (10 mL×2), dried over Na₂SO₄ and concentrated to dryness. The resulting residue was purified by Prep-HPLC (Method A) to give the titled compound (8 mg, 16%). ¹H NMR (500 MHz, DMSO-d₆) δ 8.00-7.93 (m, 2H), 7.87 (d, J=8.3 Hz, 1H), 7.50-7.36 (m, 1H), 5.61 (d, 0.1=3.2 Hz, 2H), 5.39 (s, 1H), 3.79 (dq, 1=19.2, 6.3 Hz, 1H), 3.40 (s, 1H), 3.27 (s, 4H), 3.17-3.05 (m, 1H), 2.97-2.76 (m, 4H), 2,56 (d, J=15.6 Hz, 1H), 2.30 (dd, J=28.5, 10.6 Hz, 1H), 2.10-1.82 (m, 1H), 1.71-1.41 (m, 3H), 1.31 (dd, J=19.5, 6.5 Hz, 3H), 1.02-0.83 (m, 3H), 0.63-0.45 (m, 3H) ppm. MS: M/e 504 (M+1)⁺.

### Compound A109: 2-(7-((2S,5R)-2,5-diethyl-4-(1-(2-ethylbenzo[d]thiazol-5-yl)ethyl)piperazin-1-yl)-4-methyl-5-oxo-4,5-dihydro-2H-pyrazolo[4,3-b]pyridin-2-y)acetonitrile

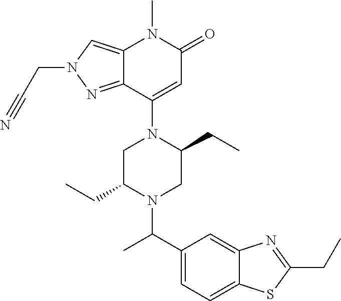 [SMILES: CCc1nc2cc(C(C)N3C[C@H](CC)N(c4cc(=O)n(C)c5cn(CC#N)nc45)C[C@H]3CC)ccc2s1]

### Step A: 1-(2-ethylbenzoldithiazol-5-yl)ethan-1-one

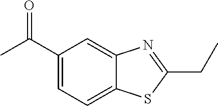

A mixture of 5-bromo-2-ethylbenzo[d]thiazole (241 mg, 1 mmol), tributyl(1-ethoxyvinyl)stannane (541 mg, 1.5 mmol) and Pd(PPh₃)₂Cl₂ (70 mg, 0.1 mmol) in toluene (5 mL) was stirred at 100° C. under N₂ for 16 hours. To the resulting solution was added HCl (0.5 mL, con.) in drops and the mixture was stirred at room temperature for 30 minutes. The mixture was treated with saturated NaHCO₃ aq. (30 ml), extracted with EtOAc(10 ml×3), washed with brine (20 mL), dried and concentrated to dryness. The resulting residue was purified by flash column chromatography (EtOAc: PE=0-50% in 30 minutes) to give the titled compound (120 mg, 58%). MS: M/e 206 (M+1)⁺.

### Step B: 1-(2-ethylbenzo[d]thiazol-5-yl)ethan-1-ol

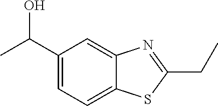

To a solution of 1-(2-ethylbenzo[d]thiazol-5-yl)ethan-1-one (120 mg, 0.58 mmol) in MeOH (3 mL) was added NaBH₄ (18 mg, 0.47 mmol) at room temperature and the mixture was stirred at room temperature for 30 minutes. The resulting mixture was treated with water (30 ml), extracted with DCM (10 mL×3). The combined organic layers were dried over Na₂SO₄ and concentrated to dryness. The resulting residue (100 mg, crude) was used in the next step. MS: M/e 208 (M+1).

### Step C: 2-(7-((2S,5R)-2,5-diethyl-4-(1-(2-ethylbenzo[d]thiazol-5-yl)ethyl)piperazin-1-yl)-4-methyl-5-oxo-4,5-dihydro-2H-pyrazolo[4,3-b]pyridin-2-yl)acetonitrile

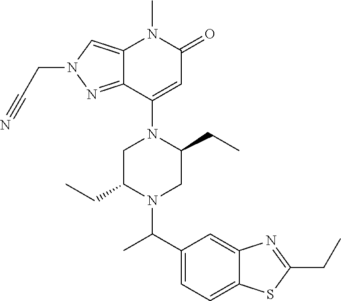 [SMILES: CCc1nc2cc(C(C)N3C[C@H](CC)N(c4cc(=O)n(C)c5cn(CC#N)nc45)C[C@H]3CC)ccc2s1]

A mixture of 1-(2-ethylbenzo[d]thiazol-5-yl)ethan-1-ol (41 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (97 mg, 0.4 mmol) and DIPEA (52 mg, 0.4 mmol) in MeCN (2 mL) was stirred at 100° C. overnight. The reaction mixture was diluted with EtOAc (20 mL), washed with water (10 mL×2), dried over Na₂SO₄ and concentrated to dryness. The resulting residue was purified by Prep-HPLC (Method A) to give the titled compound (8 mg, 15%). ¹H NMR (400 MHz, DMSO-d₆) δ 8.03-7.91 (m, 2H), 7.87 (s, 1H), 7.51-7.33 (m, 1H), 5.59 (s, 2H), 5.37 (s, 1H), 3.91-3.66 (m, 1H), 3.38 (s, 1H), 3.25 (s, 4H), 3.10 (dt, J=7.3, 4.6 Hz, 3H), 2.97-2.75 (m, 1H), 2,53 (d, J=10.9 Hz, 1H), 2.35-2.21 (m, 1H), 2.10-1.78 (m, 1H), 1.69-1.43 (m, 3H), 1.40-1.15 (m, 6H), 0.99-0.80 (m, 3H), 0.63-0.41 (m, 3H) ppm. MS: M/e 518 (M+1)⁺.

### Compound A110: 2-(7-((2S,5R)-2,5-diethyl-4-(1-(4-fluorobenzo[d]thiazol-5-yl)ethyl)piperazin-1-yl)-4-methyl-5-oxo-4,5-dihydro-2H-pyrazolo[4,3-b]pyridin-2-yl)acetonitrile

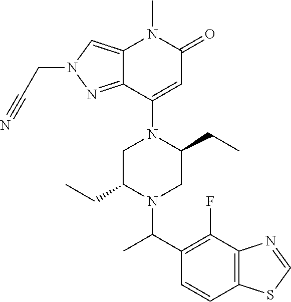 [SMILES: CC[C@H]1CN(C(C)c2ccc3scnc3c2F)[C@H](CC)CN1c1cc(=O)n(C)c2cn(CC#N)nc12]

### Step A: N-((3-bromo-2-fluorophenyl)carbamothioyl))benzamide

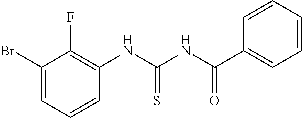 [SMILES: O=C(NC(=S)Nc1cccc(Br)c1F)c1ccccc1]

A solution of 3-bromo-2-fluoroaniline (5 g, 26.32 mmol) and benzoyl isothiocyanate (4.5 g, 27.61 mmol) in MeCN (50 ml) was stirred at rt for 30 min. The reaction was filtered. The filter cake was dried to give the titled compound (7.5 g, 81%). MS: M/e 353 (M+1)⁺.

### Step B: 1-(3-bromo-2-fluorophenyl)thiourea

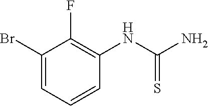 [SMILES: NC(=S)Nc1cccc(Br)c1F]

A solution of N-((3-bromo-2-fluorophenyl)carbamothioyl)benzamide (7.5 g, 21.31 mmol) and NaOH (2,56 g, 64 mmol) in MeOH (80 ml) and H₂O (20 ml) was stirred at 70° C. for 15 min. The reaction was concentrated to dryness under reduced pressure. The resulting solid was slurried in DCM:MeOH (10:1, 50 ml) and then filtered. The filtrate was evaporated to give the product (4 g, 75%). MS: M/e 249 (M+1)⁺.

### Step C: 5-bromo-4-fluorobenzo[d]thiazol-2-amine

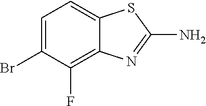 [SMILES: Nc1nc2c(F)c(Br)ccc2s1]

To a solution of 1-(3-bromo-2-fluorophenyl)thiourea (248 mg, 1 mol) in CHCl₃ (2 mL) was added Br₂ (192 mg, 1,2 mmol). The reaction mixture was stirred at 60° C. overnight. The mixture was slowly added H₂O (20 ml) and extracted with EA (20 mL×3). The organic phase was dried with Na₂SO₄ and concentrated by using a rotary evaporator to give a residue. The resulting residue was purified by flash column chromatography to give the titled compound (200 mg, 81.6%). MS: M/e 247 (M+1)⁺.

### Step D: 5-bromo-4-fluorobenzoldithiazole

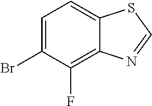 [SMILES: Fc1c(Br)ccc2scnc12]

To a solution of 5-bromo-4-fluorobenzo[d]thiazol-2-amine (110 mg, 0.45 mol) in THF (10 mL) was added Isopentyl nitrite (131 mg, 1.12 mmol). The reaction mixture was stirred at 70° C. overnight. The mixture was slowly added H₂O (20 ml) and extracted with EA (20 mL×3). The organic phase was dried with Na₂SO₄ and concentrated by using a rotary evaporator to give a residue. The resulting residue was purified by flash column chromatography to give the titled compound (60 mg, 58.3%). MS: M/e 232 (M+1)⁺.

### Step E: 1-(4-fluorobenzo[d]thiazol-5-yl)ethan-1-one

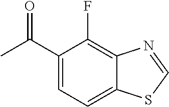

To a solution of 5-bromo-4-fluorobenzo[d]thiazole (150 mg, 0.65 mol) in toluene (10 mL) was added Pd(PPh₃)₂Cl₂ (70 mg, 0.1 mol) and tributyl(1-ethoxyvinyl)stannane (365 mg, 1 mmol). The reaction mixture was protected by N2 atmosphere and stirred at 90° C. for 16 hrs. The mixture was added 4N HCl in dionxane (0.5 ml) and stirred at RT for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (120 mg, 95.2%). MS: M/e 196 (M+1)⁺.

### Step F: 144-fluorobenzo[d]thiazol-5-yl)ethan-1-ol

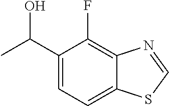

To a solution of 1-(4-fluorobenzo[d]thiazol-5-yl)ethan-1-one (120 mg, 0.65 mol) in MeOH was added NaBH₄ (23 mg, 0.6 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (100 mg, 83.3%). MS: M/e 198 (M+1)⁺.

### Step G: 2-(7-((2S,5R)-2,5-diethyl-4-(1-(4-fluorobenzoldithiazol-5-yl)ethyl)ioerazin-1-yl)-4-methyl-5-oxo-4,5-dihydro-2H-pyrazolo[4,3-b]pyridin-2-yl)acetonitrile

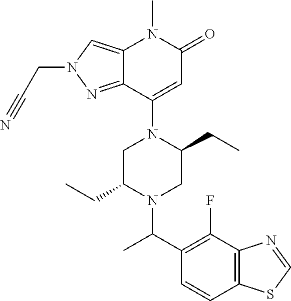 [SMILES: CC[C@H]1CN(C(C)c2ccc3scnc3c2F)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(4-fluorobenzo[d]thiazol-5-yl)ethan-1-ol (50 mg, 0.25 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method A) to give the titled Compound A110a (5 mg) and Compound A110b (4 mg).

Compound A110a (the earlier peak): ¹H NMR (500 MHz, CD₃OD) δ 9.27 (s, 1H), 7.90 (m, 2H), 7.71 (m, 1H), 5.55 (s, 1H), 5.46 (s, 2H), 4.40 (q, J=6.4 Hz, 1H), 3.43 (s, 3H), 3.32 (s, 2H), 3.27 (s, 1H), 3.04 (d, J=11.8 Hz, 1H), 2.95 (dd, J=12.0, 3.7 Hz, 1H), 2,40 (d, J=8.1 Hz, 1H), 2.11 (dt, J=15.0, 7.4 Hz, 1H), 1.83 (m, 1H), 1.57 (dd, J=16.1, 7.5 Hz, 2H), 1.44 (d, J=6.5 Hz, 3H), 0.97 (t, J=7.4 Hz, 3H), 0.71 (t, J=7.3 Hz, 3H) ppm. MS: M/e 508 (M+1)⁺.

Compound A110b (the later peak): ¹H NMR (400 MHz, CD₃OD) δ 9.26 (s, 1H), 7.93 (s, 1H), 7.87 (d, J=8.5 Hz, 1H), 7.79 (m, 1H), 5.55 (s, 1H), 5.47 (s, 2H), 4.26 (d, J=6.4 Hz, 1H), 3.51 (m, 1H), 3.43 (s, 3H), 3.33 (s, 1H), 3.23 (s, 1H), 3.20 (d, J=10.2 Hz, 1H), 2.74 (d, J=10.4 Hz, 1H), 2.36 (d, J=12.1 Hz, 1H), 1.97 (s, 1H), 1.66 (m, 3H), 1.41 (d, 0.1=6.5 Hz, 3H), 1.05 (t, J=7.2 Hz, 3H), 0.60 (t, J=7.2 Hz, 3H) ppm. MS: M/e 508 (M+1)⁺.

### Compound A111: 2-(but-2-yn-1-yl)-7-((2S,5R)-5-ethyl-2-methyl-4-(1-(3-methylquinoxalin-2-yl)ethyl)piperazin-1-yl)-4-methyl-2,4-dihydro-5H-pyrazolo[4,3-b]pyridin-5-one

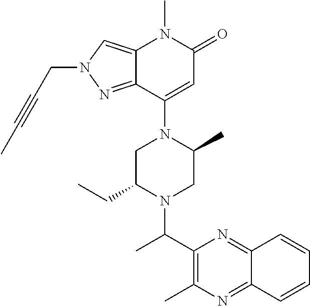 [SMILES: CC#CCn1cc2c(n1)c(N1C[C@@H](CC)N(C(C)c3nc4ccccc4nc3C)C[C@@H]1C)cc(=O)n2C]

### Step A: N-methoxy-N,3-dimethylquinoxaline-2-carboxamide

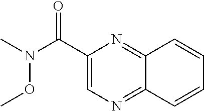 [SMILES: CON(C)C(=O)c1cnc2ccccc2n1]

To a solution of 3-methylquinoxaline-2-carboxylic acid (0.5 g, 2.6 mmol) in DMF (10 mL) were added HATU (5 g, 13.2 mmol), DIPEA (1.8 g, 14.4 mol) and N,N-dimethyl-hydroxylamine hydrochloride salt (1.3 g, 13.2 mol). The mixture was stirred at room temperature for overnight. The reaction was diluted with DCM and washed with water. The organic layer was separated, dried by Na₂SO₄, filtered, and concentrated to dryness. The resulting residue was purified by flash column chromatography to give the titled compound (400 mg, 65%). MS: M/e 232 (M+1)⁺.

### Step B: 1-(3-methylquinoxalin-2-yl)ethan-1-one

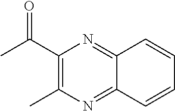

A mixture of N-methoxy-N,3-dimethylquinoxaline-2-carboxamide (400 mg, 1.73 mmol) in THF (6 mL) at 0° C. was added methyl magnesium bromide (1.15 mL, 3M, 3.4 mol). The reaction mixture was stirred at room temperature for 1 hour. The reaction was quenched by adding aqueous NH₄Cl. The resulting mixture was extracted with EA, and then concentrated to dryness. The resulting residue was purified by flash column chromatography to give the titled compound (270 mg, 84%). MS: We 187 (M+1)⁺.

### Step C: 1-(3-methylquinoxalin-2-yl)ethan-1-ol

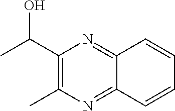

To a solution of 1-(3-methylquinoxalin-2-yl)ethan-1-one (270 mg, 1.4 mmol) in MeOH (5 mL) was added NaBH₄ (38 mg, 1 mmol) at room temperature and the resulting mixture was stirred at room temperature for 5 mins. The reaction mixture was diluted with DCM and washed with water, dried over Na₂SO₄ and concentrated to give the titled compound (250 mg). MS: M/e 189 (M+1)⁺.

### Step D: 2-(but-2-yn-1-yl)-7-((2S,5R)-5-ethyl-2-methyl-4-(1-(3-methylquinoxalin-2-yl)ethyl)piperazin-1-yl)-4-methyl-2,4-dihydro-5H-pyrazol[4,3-b]pyridin-5-one

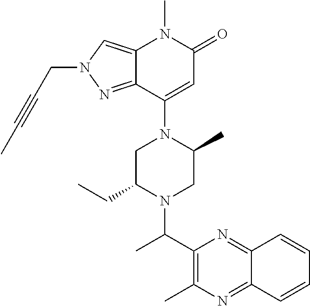 [SMILES: CC#CCn1cc2c(n1)c(N1C[C@@H](CC)N(C(C)c3nc4ccccc4nc3C)C[C@@H]1C)cc(=O)n2C]

To a solution of 2-(but-2-yn-1-yl)-7-((2S,5R)-5-ethyl-2-methylpiperazin-1-yl)-4-methyl-2,4-dihydro-5H-pyrazolo[4,3-b]pyridin-5-one (50 mg, 0.15 mmol) in CH₃CN (2 mL) and was added 1-(3-methylquinoxalin-2-yl)ethan-1-ol (60 mg, 0.3 mmol), (cyanomethyl)trimethylphosphonium iodide (110 mg, 0.45 mmol) and DIPEA(194 mg, 1.5 mmol).The resulting mixture was stirred at 105° C. overnight. The reaction solvent was removed under reduced pressure and purified by flash column chromatography (DCM: McOH) to give the titled compound, which was further separated into Compound A111a (8 mg) and Compound All1b (10 mg) by Prep-HPLC (Method A).

Compound A11a (the earlier peak): ¹H NMR (400 MHz, CD₃OD) δ 8.11-8.05 (m, 1H), 8.02-7.95 (m, 1H), 7.89 (s, 1H), 7.82-7.71 (m, 2H), 5.48 (s, 1H), 5.18 (s, 1H), 5.04 (q, J=2,4 Hz, 2H), 4.42 (d, 0.1=6.4 Hz, 1H), 4.26 (s, 1H), 3.60 (dt, J=10.0, 5.1 Hz, 1H), 3.43 (s, 3H), 3.11 (t, J=12.9 Hz, 1H), 2.92 (s, 3H), 2.86 (d, J=23.5 Hz, 1H), 2.37 (d, J=8.8 Hz, 1H), 1.86 (t, J=2,5 Hz, 3H), 1.77-1.61 (m, 2H), 1.57 (d, J=6.8 Hz, 3H), 1.16 (d, J=6.4 Hz, 3H), 1.05 (t, J=7.3 Hz, 3H) ppm. MS: M/e 498 (M+1)⁺.

Compound A111b (the later peak): ¹H NMR (400 MHz, CD₃OD) δ 8.09-8.01 (m, 1H), 7.99-7.94 (m, 1H), 7.87 (s, 1H), 7.81-7.71 (m, 2H), 5.53 (s, 1H), 5.01 (q, J=2,4 Hz, 2H), 4.88 (s, 1H), 4.59-4.43 (m, 2H), 3.47 (dd, J=13.4, 2,5 Hz, 1H), 3.43 (s, 3H), 3.12 (dd, J=12.1, 4.2 Hz, 1H), 2.94 (s, 3H), 2.84-2.66 (m, 2H), 1.84 (t, J=2,5 Hz, 3H), 1.71-1.63 (m, 1H), 1.62 (d, J=6.7 Hz, 3H), 1.42 (dd, J=13.4, 6.0 Hz, 1H), 1.04 (d, J=6.4 Hz, 3H), 0.75 (t, J=7.4 Hz, 3H) ppm. MS: We 498 (M+1)⁺.

Compound A112: 7-((2S,5R)-4-(1-(benzo[d]thiazol-6-yl)ethyl)-5-ethyl-2-methylpiperazin-1-yl)-2-(but-2-yn-1-yl)-4-methyl-2,4-dihydro-5H-pyrazolo[4,3-b]pyridin-5-one

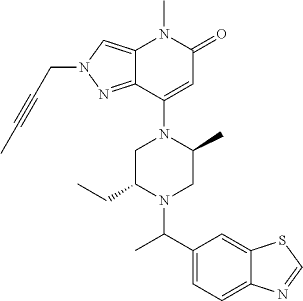 [SMILES: CC#CCn1cc2c(n1)c(N1C[C@@H](CC)N(C(C)c3ccc4ncsc4c3)C[C@@H]1C)cc(=O)n2C]

### Step A: 1-(benzo[d]thiazol-6-yl)ethan-1-ol

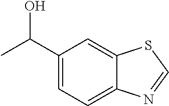

To a solution of 1-(benzo[d]thiazol-6-yl)ethan-1-one (500 mg, 2.8 mmol) in MeOH (3 mL) was added NaBH₄ (75 mg, 1.9 mmol) at room temperature and the resulting mixture was stirred at room temperature for 5 min. The reaction mixture was diluted with DCM and washed with water, dried over Na₂SO₄ and concentrated to dryness. The resulting residue was purified by flash column chromatography to give the titled compound (350 mg, 69%). MS: M/e 180 (M+1)⁺.

### Step B: 7-((2S,5R)₄-(1-(benzo[d]thiazol-6-yl)ethyl)-5-ethyl-2-methylpiperazin-1-yl)-2-(but-2-yn-1-yl)-4-methyl-2,4-dihydro-5H-pyrazolo[4,3-b]pyridin-5-one

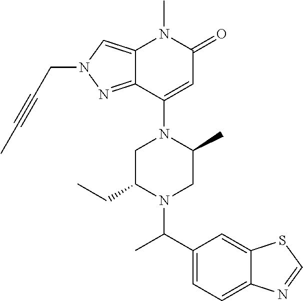 [SMILES: CC#CCn1cc2c(n1)c(N1C[C@@H](CC)N(C(C)c3ccc4ncsc4c3)C[C@@H]1C)cc(=O)n2C]

To a solution of 2-(but-2-yn-1-yl)-7-((2S,5R)-5-ethyl-2-methylpiperazin-1-yl)-4-methyl-2,4-dihydro-5H-pyrazolo[4,3-b]pyridin-5-one (50 mg, 0.15 mmol) in CH₃CN (2 mL) and was added 1-(benzo[d]thiazol-6-yl)ethan-1-ol (54 mg, 0.3 mmol), (cyanomethyl)trimethylphosphonium iodide (111 mg, 0.45 mmol) and DIPEA(197 mg, 1.5 mmol).The resulting mixture was stirred at 105° C. overnight. The reaction solvent was removed under reduced pressure. The resulting residue was purified by flash column chromatography to give the titled compound(crude), which was further purified to give the titled Compound 112a (5 mg) and Compound 112b (5 mg) by Prep- HPLC (Method A).

Compound 112a (the earlier peak): ¹H NMR (400 MHz, CD₃OD) δ 9.22 (s, 1H), 8.07 (dd, J=15.2, 4.8 Hz, 2H), 7.88 (s, 1H), 7.64 (dd, J=8.5, 1.5 Hz, 1H), 5.55 (s, 1H), 5.01 (dd, J=4.8, 2,4 Hz, 2H), 4.78-4.50 (m, 2H), 3.97 (d, J=6.7 Hz, 1H), 3.44 (s, 3H), 3.32 (dd, J=10.1, 2.2 Hz, 1H), 3.05-2.85 (m, 2H), 2,46 (s, 1H), 1.84 (t, J=2,5 Hz, 3H), 1.58 (p, J=7.1 Hz, 2H), 1.48-1.38 (m, 6H), 0.72 (t, J=7.4 Hz, 3H) ppm. MS: M/e 489 (M+1)⁺.

Compound 112b (the later peak): ¹H NMR (400 MHz, CD₃OD) δ 9.20 (s, 1H), 8.10 (s, 1H), 8.02 (d, J=8.5 Hz, 1H), 7.89 (s, 1H), 7.67 (dd, J=8.5, 1.5 Hz, 1H), 5.54 (s, 1H), 5.04 (q, J=2,4 Hz, 2H), 4.76-4.15 (m, 2H), 3.81 (q, J=6.3 Hz, 1H), 3.55 (d, J=11.1 Hz, 1H), 3.44 (s, 3H), 3.21-3.14 (m, 1H), 2.78 (dd, J=12.1, 4.1 Hz, 1H), 2.25 (d, J=11.2 Hz, 1H), 1.86 (t, J=2,5 Hz, 3H), 1.79-1.51 (m, 2H), 1.40 (d, J=6.6 Hz, 3H), 1,21 (d, J=6.6 Hz, 3H), 1.06 (t, J=7.4 Hz, 3H) ppm. MS: M/e 489 (M+1)⁺.

### Compound A113: 7-((2S,5R)-4-(1-(benzo[d]thiazol-6-yl)ethyl)-2,5-diethylpiperazin-1-yl)-2-(but-2-yn-1-yl)-4-methyl-2,4-dihydro-5H-pyrazolo[4,3-b]pyridin-5-one

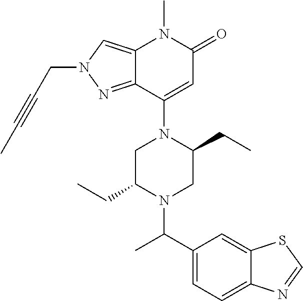 [SMILES: CC#CCn1cc2c(n1)c(N1C[C@@H](CC)N(C(C)c3ccc4ncsc4c3)C[C@@H]1CC)cc(=O)n2C]

To a solution of 2-(but-2-yn-1-yl)-7-((2S,5R)-2,5-diethylpiperazin-1-yl)-4-methyl-2,4-dihydro-5H-pyrazolo[4,3-b]pyridin-5-one (50 mg, 0.15 mmol) in CH₃CN (2 mL) and was added 1-(benzo[d]thiazol-6-yl)ethan-1-ol (53 mg, 0.3 mmol), (cyanomethyl)trimethylphosphonium iodide (111 mg, 0.45 mmol) and DIPEA(197 mg, 1.5 mmol).The resulting mixture was stirred at 105° C. overnight. The reaction solvent was removed under reduced pressure. The resulting residue was purified by flash column chromatography to give the titled compound(crude), which was further purified to give the titled Compound A113a (5 mg) and Compound A113b (6 mg) by Prep-HPLC (Method A).

Compound A113a (the earlier peak): ¹H NMR (400 MHz, CD₃OD) δ 9.21 (s, 1H), 8.10-7.99 (m, 2H), 7.88 (s, 1H), 7.63 (d, J=8.3 Hz, 1H), 5.52 (s, 1H), 5.02 (s, 2H), 4.83-4.24 (m, 1H), 3.93 (d, J=6.5 Hz, 1H), 3.43 (s, 3H), 3.31 (s, 1H), 3.05 (d, J=10.9 Hz, 1H), 2.90 (d, J=8.3 Hz, 1H), 2.65 (s, 1H), 2,40 (s, 1H), 2.13 (d, .=6.5 Hz, 1H), 1.90-1.75 (m, 4H), 1.56 (d, J=7.5 Hz, 2H), 1.41 (d, J=6.4 Hz, 3H), 0.97 (t, J=7.1 Hz, 3H), 0.66 (t, J=7.5 Hz, 3H) ppm. MS: M/e 503 (M+1)⁺.

Compound A113b (the later peak): ¹H NMR (400 MHz, CD₃OD) δ 9.20 (s, 1H), 8.11 (s, 1H), 8.02 (d, J=8.5 Hz, 1H), 7.89 (s, 1H), 7.65 (d, J=8.3 Hz, 1H), 5.51 (s, 1H), 5.03 (s, 2H), 4.82-4.41 (m, 1H), 3.79 (d, J=6.4 Hz, 1H), 3.52 (d, J=12,5 Hz, 1H), 3.44 (s, 3H), 3.27-3.10 (m, 2H), 2.75-2.61 (m, 1H), 2.36 (d, J=11.5 Hz, 1H), 2.15-1.92 (m, 1H), 1.85 (s, 3H), 1.75-1.51 (m, 3H), 1.39 (d, J=6.4 Hz, 3H), 1.04 (t, J=7.1 Hz, 3H), 0.57 (t, J=7.3 Hz, 3H) ppm. MS: M/e 503 (M+1)⁺.

### Compound A114: 2-(but-2-yn-1-yl)-7-((2S,5R)-2,5-diethyl-4-(1-(2-methylbenzo[d]thiazol-6-yl)ethyl)piperazin-1-yl)-4-methyl-2,4-dihydro-5H-pyrazolo[4,3-b]pyridin-5-one

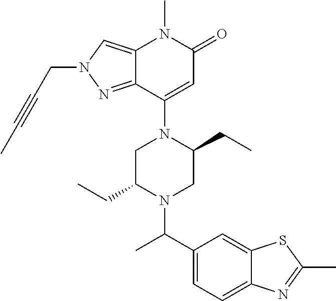 [SMILES: CC#CCn1cc2c(n1)c(N1C[C@@H](CC)N(C(C)c3ccc4nc(C)sc4c3)C[C@@H]1CC)cc(=O)n2C]

To a solution of 2-(but-2-yn-1-yl)-7-((2S,5R)-2,5-diethylpiperazin-1-yl)-4-methyl-2,4-dihydro-5H-pyrazolo[4,3-b]pyridin-5-one (163 mg, 0.5 mmol), 1-(2-methylbenzo[d]thiazol-6-yl)ethan-1-ol (200 mg, 1.1 mmol) and (cyanomethyl)trimethylphosphonium iodide (361 mg, 1.5 mmol) in CH₃CN (10 mL) was added DIPEA (387 mg, 3 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method B) to give the titled Compound A114a (35 mg) and Compound A114b (39 mg).

Compound A114a (the earlier peak): ¹H NMR (400 MHz, CD₃OD) δ 7.90 (s, 1H), 7.85 (d, J=13.7 Hz, 2H), 7.52 (d, J=8.1 Hz, 1H), 5.50 (s, 1H), 5.00 (s, 2H), 3.86 (s, 1H), 3.42 (s, 4H), 3.32 (s, 1H), 3.24 (m, 1H), 3.02 (d, J=11.7 Hz, 1H), 2.87 (d, J=12,4 Hz, 1H), 2.81 (s, 3H), 2.38 (s, 1H), 2.11 (s, 1H), 1.82 (s, 4H), 1.52 (s, 2H), 1.38 (d, J=5.2 Hz, 3H), 0.94 (s, 3H), 0.65 (s, 3H) ppm. MS: M/e 517 (M+1)⁺.

Compound A114b (the later peak): ¹H NMR (400 MHz, CD₃OD) δ 7.94 (s, 1H), 7.87 (s, 1H), 7.81 (d, J=8.3 Hz, 1H), 7.54 (d, J=8.3 Hz, 1H), 5.49 (s, 1H), 5.01 (s, 2H), 3.72 (d, J=5.3 Hz, 1H), 3.49 (d, J=14.9 Hz, 1H), 3.42 (s, 3H), 3.32 (s, 1H), 3.23 (m, 1H), 3.14 (m, 1H), 2.80 (s, 3H), 2.64 (s, 1H), 2.34 (d, 0.1=11.7 Hz, 1H), 1.97 (s, 1H), 1.83 (s, 3H), 1.61 (d, J=19.5 Hz, 3H), 1.35 (d, J=5.9 Hz, 3H), 1.01 (s, 3H), 0.55 (s, 3H) ppm. MS: M/e 517 (M+1)⁺.

Compound A115: 2-(but-2-yn-1-yl)-7-((2S,5R)-5-ethyl-2-methyl-4-(1-(2-methylbenzo[d]thiazol-6-yl)ethyl)piperazin-1-yl)-4-methyl-2,4-dihydro-5H-pyrazolo[4,3-b]pyridin-5-one

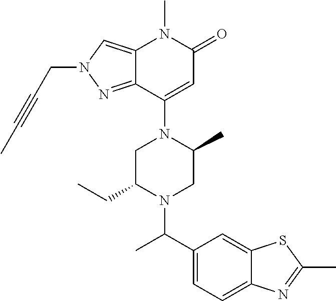 [SMILES: CC#CCn1cc2c(n1)c(N1C[C@@H](CC)N(C(C)c3ccc4nc(C)sc4c3)C[C@@H]1C)cc(=O)n2C]

To a solution of 2-(but-2-yn-1-yl)-7-((2S,5R)-5-ethyl-2-methylpiperazin-1-yl)-4-methyl-2,4-dihydro-5H-pyrazolo[4,3-b]pyridin-5-one (150 mg, 0.5 mmol), 1-(2-methylbenzo[d]thiazol-6-yl)ethan-1-ol (192 mg, 1 mmol) and (cyanomethyl)trimethylphosphonium iodide (361 mg, 1.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by Prep-HPLC (Method A) to give the titled Compound A115a (13 mg) and Compound A115b (16 mg).

Compound A115a (the earlier peak): ¹H NMR (400 MHz, CD₃OD) δ 7.93 (s, 1H), 7.87 (m, 2H), 7.54 (d, J=8.4 Hz, 1H), 5.54 (s, 1H), 5.01 (s, 2H), 4.64 (d, J=38.4 Hz, 1H), 3.89 (d, J=6.6 Hz, 1H), 3.45 (d, J=14.0 Hz, 4H), 3.34 (s, 1H), 2.96 (s, 1H), 2.85 (d, J=20.4 Hz, 4H), 2,43 (s, 1H), 1.84 (s, 3H), 1.56 (m, 2H), 1.40 (m, 6H), 0.71 (t, .=7.2 Hz, 3H) ppm. MS: M/e 503 (M+1)⁺.

Compound A115b (the later peak): ¹H NMR (400 MHz, CD₃OD) δ 7.95 (s, 1H), 7.89 (s, 1H), 7.82 (d, J=8.4 Hz, 1H), 7.57 (d, J=8.4 Hz, 1H), 5.53 (s, 1H), 5.04 (d, J=1.8 Hz, 2H), 4.42 (s, 1H), 3.75 (d, J=6.4 Hz, 1H), 3.53 (d, .=12.3 Hz, 1H), 3.44 (s, 3H), 3.34 (s, 1H), 3.17 (d, J=10.1 Hz, 1H), 2.82 (s, 3H), 2.75 (dd, J=12.0, 3.5 Hz, 1H), 2.23 (d, J=12.0 Hz, 1H), 1.85 (s, 3H), 1.69 (m, 1H), 1.57 (dd, J=13.3, 7.3 Hz, 1H), 1.37 (d, J=6.4 Hz, 3H), 1,20 (d, J=6.5 Hz, 3H), 1.05 (t, J=7.3 Hz, 3H) ppm. MS: M/e 503 (M+1)⁺.

### Compound A116: 2-(but-2-yn-1-yl)-7-((2S,5R)-5-ethyl-4-(1-(7-fluoro-2-methylbenzo[d]thiazol-6-yl)ethyl)-2-methylpiperazin-1-yl)-4-methyl-2,4-dihydro-5H-pyrazolo[4,3-b]pyridin-5-one

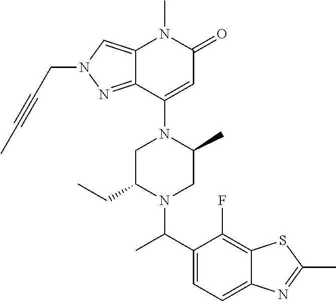 [SMILES: CC#CCn1cc2c(n1)c(N1C[C@@H](CC)N(C(C)c3ccc4nc(C)sc4c3F)C[C@@H]1C)cc(=O)n2C]

A solution of 2-(but-2-yn-1-yl)-7-((2S,5R)-5-ethyl-2-methylpiperazin-1-yl)-4-methyl-2,4-dihydro-5H-pyrazolo[4,3-b]pyridin-5-one (50 mg, 0.15 mmol), 1-(7-fluoro-2-methylbenzo[d]thiazol-6-yl)ethan-1-ol (64.5 mg, 0.31 mmol), (cyanomethyl)trimethylphosphonium iodide (111.5 mg, 0.46 mmol) and DIPEA (197.2 mg, 1.53 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography to give the titled compound, which was further separated into Compound A116a (16.36 mg) and Compound A116b (10.57 mg) by Prep-HPLC (Method A).

Compound A116a (the earlier peak): ¹H NMR (400 MHz, CD₃OD) δ 7.89 (s, 1H), 7.76 (d, J=8.5 Hz, 1H), 7.69 (t, J=7.5 Hz, 1H), 5.54 (s, 1H), 5.02 (s, 2H), 4.33 (q, J=6.5 Hz, 1H), 3.44 (s, 3H), 3.32 (s, 2H), 3.27 (m, 1H), 3.01 (dd, J=11.99, 4.00 Hz, 1H), 2.90-2.79 (m, 4H), 2,40 (s, 1H), 1.84 (s, 3H), 1.63-1.51 (m, 2H), 1.43 (d, J=6.5 Hz, 3H), 1.38 (d, J=6.4 Hz, 3H), 0.75 (t, 0.1=7.2 Hz, 3H) ppm. MS: M/e 521 (M+1)⁺.

Compound A116b (the later peak): ¹H NMR (400 MHz, CD₃OD) δ 7.90 (s, 1H), 7.82-7.75 (m, 1H), 7.72 (d, J=8.5 Hz, 1H), 5.54 (s, 1H), 5.04 (s, 2H), 4.17 (q, J=6.6 Hz, 1H), 3.52 (d, J=13.4 Hz, 1H), 3.44 (s, 3H), 3.32 (s, 2H), 3.17 (d, J=9.3 Hz, 1H), 2.89-2.77 (m, 4H), 2.21 (d, J=11.9 Hz, 1H), 1.86 (s, 3H), 1.80-1.66 (m, 1H), 1.63-1.51 (m, 1H), 1.40 (d, J=6.6 Hz, 3H), 1,22 (d, J=6.4 Hz, 3H), 1.06 (t, J=7.3 Hz, 3H) ppm. MS: M/e 521 (M+1)⁺.

### Compound A117: 2-(but-2-yn-1-yl)-7-((2S,5R)-2,5-diethyl-4-(1-(thiazolo[5,4-b]pyridin-5-yl)ethyl)piperazin-1-yl)-4-methyl-2,4-dihydro-5H-pyrazolo[4,3-b]pyridin-5-one

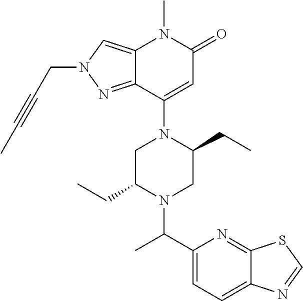 [SMILES: CC#CCn1cc2c(n1)c(N1C[C@@H](CC)N(C(C)c3ccc4ncsc4n3)C[C@@H]1CC)cc(=O)n2C]

To a solution of 2-(but-2-yn-1-yl)-7-((2S,5R)-2,5-diethylpiperazin-1-yl)-4-methyl-2,4-dihydro-5H-pyrazolo[4,3-b]pyridin-5-one (100 mg, 0.29 mmol) in CH₃CN (5 mL) and was added 1-(thiazolo[5,4-b]pyridin-5-yl)ethan-1-ol (158 mg, 0.88 mmol), (cyanomethyl)trimethylphosphonium iodide (213 mg, 0.88 mmol) and DIPEA(0.38 g, 3.0 mmol).The resulting mixture was stirred at 105° C. overnight. The reaction solvent was removed under reduced pressure. The resulting residue was purified by flash column chromatography to give the titled compound(crude), which was further purified to give the titled Compound A117a (37 mg) and Compound A117b (41 mg) by Prep-chiral HPLC. The chiral separation conditions are shown below.

| Column | IE |
|---|---|
| Column Size | 20.0 mm × 250 mm, 5 um |
| Mobile Phase A | HEXANE 30% |
| Mobile Phase B | ETOH(0.2% 2M NH₃MEOH) 70% |
| Run Time | 15 min |
| Flow Rate | 18 mL/min |
| Wave Length | UV 210 nm and 310 nm |
| Temperature | RT |
| Prep-HPLC Equipment | Prep-HPLC-Gilson |

Compound A117a (the earlier peak): ¹H NMR (400 MHz, CD₃OD) δ 9.34 (s, 1H), 8.43 (d, J=8.5 Hz, 1H), 7.88 (s, 1H), 7.80 (d, J=8.5 Hz, 1H), 5.52 (s, 1H), 5.02 (d, J=2.3 Hz, 2H), 4.75-4.23 (m, 2H), 4.11 (q, J=6.6 Hz, 1H), 3.43 (s, 3H), 3.34 (d, J=6.2 Hz, 1H), 3.06-2.94 (m, 2H), 2.38 (d, J=8.2 Hz, 1H), 2.08 (dt, J=23.2, 7.5 Hz, 1H), 1.92-1.76 (m, 4H), 1.72-1.51 (m, 2H), 1.45 (d, J=6.6 Hz, 3H), 0.94 (t, J=7.4 Hz, 3H), 0.70 (t, J=7.4 Hz, 3H) ppm. MS: M/e 504 (M+1)⁺.

Compound A117b (the later peak): ¹H NMR (400 MHz, CD₃OD) δ 9.34 (s, 1H), 8.41 (d, J=8.5 Hz, 1H), 7.89 (s, 1H), 7.85 (d, J=8.6 Hz, 1H), 5.52 (s, 1H), 5.03 (d, J=2.3 Hz, 2H), 4.76-4.02 (m, 2H), 3.92 (q, J=6.6 Hz, 1H), 3.54 (d, J=12,4 Hz, 1H), 3.42 (s, 3H), 3.17 (d, J=10.1 Hz, 1H), 2.78 (dd, J=12.3, 3.5 Hz, 1H), 2.26 (d, J=11.9 Hz, 1H), 2.05-1.93 (m, 1H), 1.85 (t, J=2.2 Hz, 3H), 1.79-1.48 (m, 3H), 1.41 (d, J=6.7 Hz, 3H), 1.03 (t, J=7.3 Hz, 3H), 0.62 (t, J=7.4 Hz, 3H) ppm. MS: M/e 504 (M+1)⁺.

### Compound A118: 2-(but-2-yn-1-yl)-7-((2S,5R)-5-ethyl-2-methyl-4-(1-(thiazolo[5,4-b]pyridin-5-yl)ethyl)piperazin-1-yl)-4-methyl-2,4-dihydro-5H-pyrazolo[4,3-b]pyridin-5-one

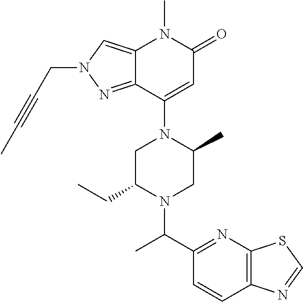 [SMILES: CC#CCn1cc2c(n1)c(N1C[C@@H](CC)N(C(C)c3ccc4ncsc4n3)C[C@@H]1C)cc(=O)n2C]

To a solution of 2-(but-2-yn-1-yl)-7-((2S,5R)-5-ethyl-2-methylpiperazin-1-yl)-4-methyl-2,4-dihydro-5H-pyrazolo[4,3-b]pyridin-5-one (50 mg, 0.15 mmol) in CH₃CN (5 mL) and was added 1-(thiazolo[5,4-b]pyridin-5-yl)ethan-1-ol (82 mg, 0.45 mmol), (cyanomethyl)trimethylphosphonium iodide (111 mg, 0.45 mmol) and DIPEA(0.2 g, 1.5 mmol).The resulting mixture was stirred at 105° C. overnight. The reaction solvent was removed under reduced pressure. The resulting residue was purified by flash column chromatography to give the titled compound(crude), which was further purified to give the titled compound (37 mg) by Prep-HPLC (Method A). ¹H NMR (400 MHz, CD₃OD) δ 9.34 (d, J=4.0 Hz, 1H), 8.48-8.37 (m, 1H), 7.94-7.77 (m, 2H), 5.54 (d, J=2.9 Hz, 1H), 5.03 (dd, J=10.3, 1.6 Hz, 2H), 4.81-4.28 (m, 1H), 4.08 (dt, J=56.4, 6.5 Hz, 1H), 3.57 (d, .=13.4 Hz, 1H), 3.44 (s, 3H), 3.35 (dd, J=17.4, 3.9 Hz, 1H), 3.24-3.02 (m, 1H), 2.94-2.82 (m, 1H), 2,52-2.14 (m, 1H), 1.85 (d, J=7.4 Hz, 3H), 1.81-1.53 (m, 2H), 1.46-1.19 (m, 6H), 1.11-0.71 (m, 3H) ppm. MS: M/e 490 (M+1)⁺.

### Compound A119: 7-((2S,5R)-4-(1-(benzo[d]thiazol-5-yl)ethyl)-5-ethyl-2-methylpiperazin-1-yl)-2-(but-2-yn-1-yl)-4-methyl-2,4-dihydro-5H-pyrazolo[4,3-b]pyridin-5-one

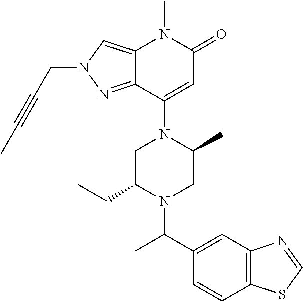 [SMILES: CC#CCn1cc2c(n1)c(N1C[C@@H](CC)N(C(C)c3ccc4scnc4c3)C[C@@H]1C)cc(=O)n2C]

To a solution of 2-(but-2-yn-1-yl)-7-((2S,5R)-5-ethyl-2-methylpiperazin-1-yl)-4-methyl-2,4-dihydro-5H-pyrazolo[4,3-b]pyridin-5-one (50 mg, 0.15 mmol) in CH₃CN (2 mL) and was added 1-(benzo[d]thiazol-5-yl)ethan-1-ol (27 mg, 0.15 mmol), (cyanomethyl)trimethylphosphonium iodide (110 mg, 0.45 mmol) and DIPEA(197 mg, 1.5 mmol).The resulting mixture was stirred at 105° C. overnight. The reaction solvent was removed under reduced pressure, The reaction was diluted with DCM and washed with water. The organic layer was separated, dried by Na₂SO₄, filtered, and concentrated to dryness. The resulting residue was purified by flash column chromatography (DCM:MeOH) to give the titled compound(crude), which was further purified to give the titled compound(2 mg) by Prep-HPLC (Method A). ¹H NMR (400 MHz, DMSO-d₆) δ 9.38 (d, J=9.7 Hz, 1H), 8.19-8.03 (m, 2H), 7.93 (d, J=4.9 Hz, 1H), 7.55 (t, J=9.1 Hz, 1H), 5.36 (d, J=3.2 Hz, 1H), 5.06 (dd, J=10.7, 1.9 Hz, 2H), 4.99-4.18 (m, 1H), 3.99-3.71 (m, 1H), 3.41 (d, J=11.3 Hz, 1H), 3.27 (s, 3H), 3.12 (dd, J=19.5, 9.7 Hz, 1H), 2.96-2,54 (m, 2H), 2,42-2.12 (m, 1H), 1.82 (dt, J=8.9, 2.3 Hz, 3H), 1.74-1.46 (m, 2H), 1.33 (dd, J=10.1, 6.5 Hz, 3H), 1.30-1.08 (m, 3H), 0.82 (dt, J=183.5, 7.3 Hz, 3H) ppm. MS: M/e 489 (M+1)⁺.

### Compound A120: 7-((2S,5R)-4-(1-(benzo[d]thiazol-5-yl)ethyl)-2,5-diethylpiperazin-1-yl)-2-(but-2-yn-1-yl)-4-methyl-2,4-dihydro-5H-pyrazolo[4,3-b]pyridin-5-one

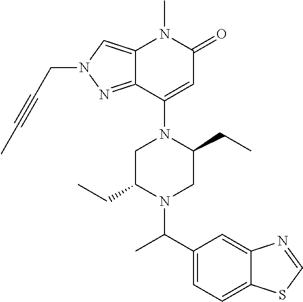 [SMILES: CC#CCn1cc2c(n1)c(N1C[C@@H](CC)N(C(C)c3ccc4scnc4c3)C[C@@H]1CC)cc(=O)n2C]

To a solution of 2-(but-2-yn-1-yl)-7-((2S,5R)-5-ethyl-2-methylpiperazin-1-yl)-4-methyl-2,4-dihydro-5H-pyrazolo[4,3-b]pyridin-5-one (250 mg, 0.73 mmol) in CH₃CN (8 mL) and was added 1-(benzo[d]thiazol-5-yl)ethan-1-ol (262 mg, 1.44 mmol), (cyanomethyl)trimethylphosphonium iodide (532 mg, 2.19 mmol) and DIPEA(0.94 g, 7.3 mmol).The resulting mixture was stirred at 105° C. overnight. The reaction solvent was removed under reduced pressure. The resulting residue was purified by flash column chromatography to give the titled compound(crude), which was further purified to give the titled Compound A120a (51 mg) and Compound A120b (54 mg) by Prep-chiral HPLC. The chiral separation conditions are shown below.

| Column | Cellulose-C |
|---|---|
| Column Size | 20.0 mm × 250 mm, 5 um |
| Mobile Phase A | HEXANE 80% |
| Mobile Phase B | ETOH(0.2% 2M NH₃MEOH) 20% |
| Run Time | 12 min |
| Flow Rate | 18 mL/min |
| Wave Length | UV 220 nm and 310 nm |
| Temperature | RT |
| Prep-HPLC Equipment | Prep-HPLC-Gilson |

Compound A120a (the earlier peak): ¹H NMR (400 MHz, CD₃OD) 59.23 (s, 1H), 8.04 (d, J=7.8 Hz, 2H), 7.86 (s, 1H), 7.57 (d, J=8.7 Hz, 1H), 5.51 (s, 1H), 5.00 (s, 2H), 4.83-4.11 (m, 2H), 3.93 (d, J=5.8 Hz, 1H), 3.42 (s, 3H), 3.25 (s, 1H), 3.04 (d, J=11.6 Hz, 1H), 2.89 (d, J=11.3 Hz, 1H), 2,48-2.35 (m, 1H), 2.24-2.05 (m, 1H), 1.89-1.83 (m, 1H), 1.82 (s, 3H), 1.63-1.44 (m, 2H), 1.40 (d, J=6.0 Hz, 3H), 0.95 (t, J=6.9 Hz, 3H), 0.65 (t, J=7.0 Hz, 3H) ppm. MS: M/e 503 (M+1)⁺.

Compound A120b (the later peak): ¹H NMR (400 MHz, CD₃OD) δ 9.22 (s, 1H), 8.09 (s, 1H), 8.01 (d, J=8.5 Hz, 1H), 7.87 (s, 1H), 7.59 (d, J=8.3 Hz, 1H), 5.49 (s, 1H), 5.02 (s, 2H), 4.83-3.98 (m, 2H), 3.78 (d, J=6.4 Hz, 1H), 3.51 (d, J=12.6 Hz, 1H), 3.42 (s, 3H), 3.18 (d, J=9.9 Hz, 1H), 2.67 (d, J=11.2 Hz, 1H), 2.37 (d, J=12,4 Hz, 1H), 1.96 (s, 1H), 1.83 (s, 3H), 1.75-1.51 (m, 3H), 1.37 (d, J=6.1 Hz, 3H), 1.02 (t, J=7.2 Hz, 3H), 0.55 (t, J=6.9 Hz, 3H) ppm. MS: M/e 503 (M+1)⁺.

### Compound A121: 7-((2S,5R)-4-(1-(6-cyclopropylpyridin-3-yl)ethyl)-2,5-diethylpiperazin-1-yl)-4-methyl-2-(oxetan-3-yl)-2,4-dihydro-5H-pyrazolo[4,3-b]pyridin-5-one

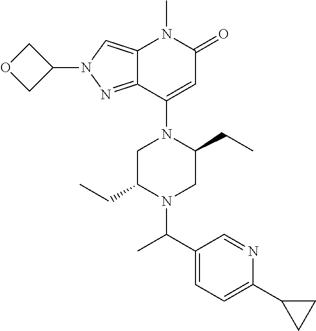 [SMILES: CC[C@H]1CN(C(C)c2ccc(C3CC3)nc2)[C@H](CC)CN1c1cc(=O)n(C)c2cn(C3COC3)nc12]

### Step A: 7-((2S,5R)-4-(I-(6-cyclonronvlpyridin-3-yl)ethyl)-2,5-diethylpiperazin-1-yl)-4-methyl-2-(tetrahydro-2H-pyran-2-yl)-2,4-dihydro-5H-pyrazolo[4,3-b]pyridin-5-one

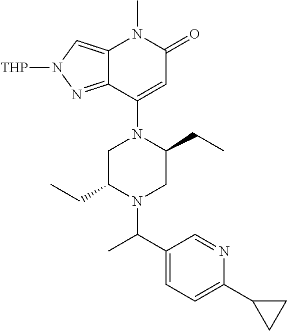

To a solution of 4-methyl-5-oxo-2-(tetrahydro-2H-pyran-2-yl)-4,5-dihydro-2H-pyrazolo[4,3-b]pyridin-7-yl trifluoromethanesulfonate (99 mg, 0.26 mmol) and (2R,5S)-1-(1-(6-cyclopropylpyridin-3-yl)ethyl)-2,5-diethylpiperazine (50 mg, 0.17 mmol) in CH₃CN (3 mL) was added DIPEA (129 mg, 1 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified by flash column chromatography to give the titled compound (60 mg, 44.7%). MS: M/e 519 (M+1)^{t}.

### Step B: 7-((2S,5R)₄-(1-(6-cyclopropylo,vridin-3-yl)ethyl-2,5-diethylpiperazin-1-yl)-4-methyl-2,4-dihydro-5H-pyrazolo[4,3-b]pyridin-5-one

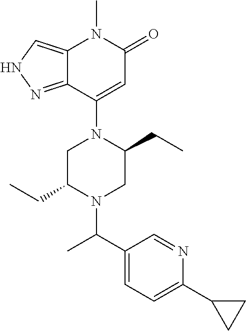 [SMILES: CC[C@H]1CN(C(C)c2ccc(C3CC3)nc2)[C@H](CC)CN1c1cc(=O)n(C)c2c[nH]nc12]

To a solution of 7-((2S,5R)-4-(1-(6-cyclopropylpyridin-3-yl)ethyl)-2,5-diethylpiperazin-1-yl)-4-methyl-2-(tetrahydro-2H-pyran-2-yl)-2,4-dihydro-5H-pyrazolo[4,3-b]pyridin-5-one (60 mg, 0.12 mol) in DCM (3 mL) was added TFA (3 ml). The reaction mixture was protected by N₂ atmosphere and stirred at RT for overnight. The mixture was concentrated in vacuo. The resulting residue was added saturated NaHCO₃ aqueous solution (10 ml) and extracted with EA (20 mL×3). The organic phase was dried with Na₂SO₄ and concentrated by using a rotary evaporator to give the titled compound (52 mg, 100%). MS: M/e 435 (M+1)⁺.

### Step C: 7-((2S,5R)-4-(1-(6-cyclouronvlpyridin-3-yl)ethyl)-25-diethylpiperazin-1-yl-4-methyl-2-(oxetan-3-yl)_{2,4}-dihydro-5H-pyrazolo[4,3-b]pyridin-5-one

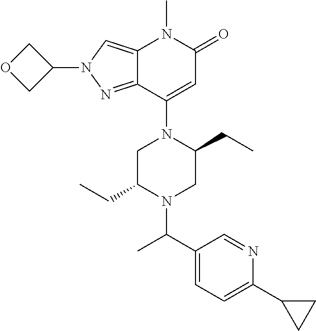 [SMILES: CC[C@H]1CN(C(C)c2ccc(C3CC3)nc2)[C@H](CC)CN1c1cc(=O)n(C)c2cn(C3COC3)nc12]

To a solution of 7-((2S,5R)-4-(1-(6-cyclopropylpyridin-3-yl)ethyl)-2,5-diethylpiperazin-1-yl)-4-methyl-2,4-dihydro-5H-pyrazolo[4,3-b]pyridin-5-one (10 mg, 0.02 mol) in DMF (10 mL) was added 3-iodooxetane (18.3 mg, 0.1 mol) and K₂CO₃ (13.9 mg, 0.1 mmol). The reaction mixture was protected by N₂ atmosphere and stirred at RT for 16 hrs. The mixture was added H₂O (10 ml) and extracted with EA (20 mL×3). The organic phase was dried with Na₂SO₄ and concentrated by using a rotary evaporator to give a residue. The resulting residue was purified by Prep-HPLC (Method A) to give the titled compound (1 mg, 11%). ¹H NMR (400 MHz, CD₃OD) δ 8.32 (d, J=14.8 Hz, 1H), 7.95 (s, 1H), 7.73 (s, 1H), 7.20 (t, J=9.4 Hz, 1H), 5.68 (s, 1H), 5.54 (s, 1H), 5.05 (d, J=21.1 Hz, 4H), 3.78-3.64 (m, 1H), 3.50 (d, J=16.8 Hz, 1H), 3.43 (s, 3H), 3.34 (s, 1H), 3.14 (d, J=11.7 Hz, 1H), 2.98 (m, 1H), 2.67 (d, J=10.6 Hz, 1H), 2.34 (d, J=13.6 Hz, 1H), 2.09 (s, 1H), 1.87 (d, J=53.4 Hz, 1H), 1.62 (d, J=50.6 Hz, 3H), 1.35 (dd, J=13.1, 6.5 Hz, 3H), 0.99 (dd, J=22,5, 15.5 Hz, 7H), 0.75 (d, J=7.0 Hz, 1.5H), 0.63 (s, 1.5H) ppm. MS: M/e 491 (M+1)⁺.

### Compound A122: 7-((2S,5R)-4-(1-(6-cyclopropylpyridin-3-yl)ethyl)-2,5-diethylpiperazin-1-yl)-4-methyl-2-(oxetan-3-ylmethyl)-2,4-dihydro-5H-pyrazolo[4,3-b]pyridin-5-one

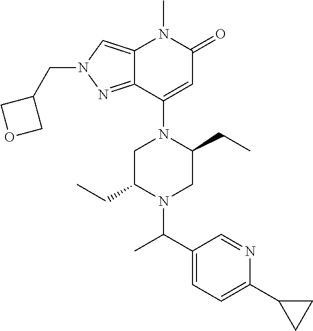 [SMILES: CC[C@H]1CN(C(C)c2ccc(C3CC3)nc2)[C@H](CC)CN1c1cc(=O)n(C)c2cn(CC3COC3)nc12]

To a solution of 7-((2S,5R)-4-(1-(6-cyclopropylpyridin-3-yl)ethyl)-2,5-diethylpiperazin-1-yl)-4-methyl-2,4-dihydro-5H-pyrazolo[4,3-b]pyridin-5-one (10 mg, 0.02 mol) in DMF (10 mL) was added 3-(bromomethyl)oxetane (7.5 mg, 0.05 mol) and K₂CO₃ (13.9 mg, 0.1 mmol). The reaction mixture was protected by N2 atmosphere and stirred at RT for 16 hrs. The mixture was added H₂O (10 ml) and extracted with EA (20 mL×3). The organic phase was dried with Na₂SO₄ and concentrated by using a rotary evaporator to give a residue. The resulting residue was purified by Prep-HPLC (Method A) to give the titled compound (1 mg). ¹H NMR (400 MHz, CD₃OD) δ 8.32 (d, J=13.7 Hz, 1H), 7.85 (d, J=1.7 Hz, 1H), 7.72 (d, J=5.7 Hz, 1H), 7.19 (m, 1H), 5.51 (s, 1H), 4.80 (t, J=7.0 Hz, 2H), 4.58 (dd, J=7.1, 2,5 Hz, 4H), 3.78 (d, .=6.4 Hz, 0.5H), 3.63 (d, J=6.6 Hz, 0.5H), 3.56 (s, 1H), 3.45 (d, J=22.3 Hz, 4H), 3.32 (s, 1H), 3.25 (s, 1H), 3.13 (s, 0.5H), 2.99 (d, J=11.9 Hz, 0.5H), 2.86 (d, J=8.6 Hz, 0.5H), 2.66 (d, J=9.5 Hz, 0.5H), 2.33 (m, 1H), 2.09 (d, J=4.4 Hz, 1H), 1.83 (dd, J=14.9, 7.8 Hz, 1H), 1.59 (dd, J=45.0, 6.7 Hz, 3H), 1.34 (dd, J=12.7, 6.5 Hz, 3H), 0.99 (m, 7H), 0.70 (t, J=7.3 Hz, 1.5H), 0.60 (t, J=7.2 Hz, 1.5H) ppm. MS: M/e 505 (M+1)⁺.

### Compound A123: 2-(7-((2S,5R)-4-(1-(benzo[d]thiazol-6-yl)ethyl)-2,5-diethylpiperazin-1-yl)-4-methyl-5-oxo-4,5-dihydro-2H-pyrazolo[4,3-d]pyrimidin-2-yl)acetonitrile

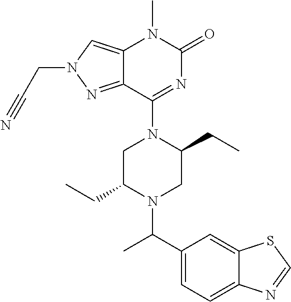 [SMILES: CC[C@H]1CN(C(C)c2ccc3ncsc3c2)[C@H](CC)CN1c1nc(=O)n(C)c2cn(CC#N)nc12]

### Step A: tert-butyl (2S,5R)-4-(1-(benzo[d]thiazol-6-yl)ethyl)-2,5-diethylpiperazine-1-carboxylate

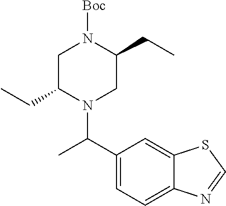 [SMILES: CC[C@H]1CN(C(C)c2ccc3ncsc3c2)[C@H](CC)CN1C(=O)OC(C)(C)C]

To a solution of tert-butyl (2S,5R)-2,5-diethylpiperazine-1-carboxylate (605 mg, 2,5 mmol), 1-(benzo[d]thiazol-6-yl)ethan-1-ol (895 mg, 5 mmol) and DIPEA (3.22 g, 25 mol) in CH₃CN (10 ml ) was added (cyanomethyl)trimethylphosphonium iodide (1.82 g, 7.5 mmol). The mixture was stirred at 103° C. in a sealed bottle for 16 hours. The reaction was cooled to room temperature, diluted by adding water, extracted with EA (80 mL×2), washed with brine, dried over Na₂SO₄, filtered and concentrated to dryness. The resulting residue was purified by flash column chromatography (EA/PE=1/1) to give the titled compound (0.93 mg, 92%, crude). MS: M/e 404 (M+1).

### Step B: 641-((2R,5S)-2,5-diethylpiperazin-1-yl)ethyl)benzo[d]thiazole

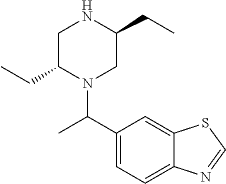 [SMILES: CC[C@H]1CN(C(C)c2ccc3ncsc3c2)[C@H](CC)CN1]

To a solution of tert-butyl (2S,5R)-4-(1-(benzo[d]thiazol-6-yl)ethyl)-2,5-diethylpiperazine-1-carboxylate (930 mg, 2.3 mmol) in DCM (10 mL) was added TMSOTf (645 mg, 2.9 mmol). The resulted mixture was stirred at room temperature for 2 hrs. The mixture was diluted with water (40 mL), extracted with DCM (40 mL). The aqueous layer was collected, basified with Na₂CO₃ solution to pH=9, extracted with DCM/MeOH (60 mL×3, 10/1), dried over Na₂SO₄, filtered, and concentrated to dryness. concentrated under reduced pressure, to give titled compound (345 mg, 49%). MS: M/e 304 (M+1)⁺.

### Step C: 7-((2S,5R)₄-(1-(benzoldithiazol-6-yl)ethyl-2,5-diethylpiperazin-1-yl)-4-methyl-2-(tetrahydro-2H-pyran-2-yl)-2,4-dihydro-5H-pyrazolo[4,3-d]pyrimidin-5-one

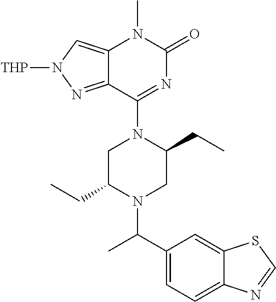

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5 mmol) in CH₃CN (3 mL) was added DIPEA (129 mg, 1 mmol), followed by POCl₃ (153 mg, 1 mmol) and catalyst amount of DMF. The reaction was stirred at 80° C. for 2 hrs. The reaction was cooled to room temperature, quenched with ice-water, extracted with EtOAc (60 mL×2), washed with brine, dried over Na₂SO₄, filtered, and concentrated to dryness.

The above residue was dissolved in CH₃CN (3 mL). 6-(1-((2R,5S)-2,5-diethylpiperazin-1-yl)ethyl)benzo[d]thiazole (150 mg, 0.5 mmol) and DIPEA (129 mg, 1 mmol) were added. The resulting mixture was heated at 85° C. for 16 hrs. The reaction was quenched with water, extracted with EtOAc (60 mL×2), washed with brine, dried over Na₂SO₄, filtered, and concentrated to dryness. The resulting residue was purified by flash column chromatography (DCM/MeOH=20/1) to give the titled compound (100 mg, 37%). MS: M/e 536 (M+1)⁺.

### Step D: 7-((2S,5R)-4-(1-(benzo[d]thiazol-6-yl)ethyl)-2,5-diethylpiperazin-1-yl) 4-methyl-2,4-dihydro-5H-pyrazolo[4,3-d]pyrimidin-5-one

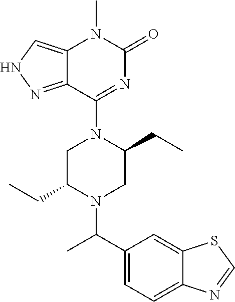 [SMILES: CC[C@H]1CN(C(C)c2ccc3ncsc3c2)[C@H](CC)CN1c1nc(=O)n(C)c2c[nH]nc12]

To a solution of 7-((2S,5R)-4-(1-(benzo[d]thiazol-6-yl)ethyl)-2,5-diethylpiperazin-1-yl)-4-methyl-2-(tetrahydro-2H-pyran-2-yl)-2,4-dihydro-5H-pyrazolo[4,3-d]pyrimidin-5-one (100 mg, 0.186 mmol) in DCM (3 mL) was added TFA (10 mL). The resulted mixture was stirred at room temperature for 16 hrs. The mixture was concentrated under reduced pressure to give titled compound (10 mg, crude). MS: M/e 452 (M+1)⁺.

### Step E: 2-(7-((2S,5R)-4-(1-(benzoldithiazol-6-yl)ethyl)-2,5-diethyluinerazin-1-yl)-4-methyl-5-oxo-4,5-dihydro-2H-pyrazolo[4,3-d]pyrimidin-2-yl)acetonitrile

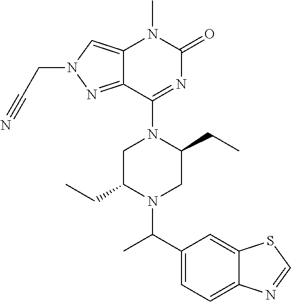 [SMILES: CC[C@H]1CN(C(C)c2ccc3ncsc3c2)[C@H](CC)CN1c1nc(=O)n(C)c2cn(CC#N)nc12]

To a mixture of 7-((2S,5R)-4-(1-(benzo[d]thiazol-6-yl)ethyl)-2,5-diethylpiperazin-1-yl)-4-methyl-2,4-dihydro-5H-pyrazolo[4,3-d]pyrimidin-5-one (100 mg, crude) and K₂CO₃ (258 mg, 1.87 mmol) in DMF (4 mL) was added 2-chloroacetonitrile (42 mg, 0.56 mmol). The reaction was stirred at room temperature for 16 hrs. The reaction was diluted with water, extracted with EtOAc (60 mL×2), washed with brine, dried over Na₂SO₄, filtered, and concentrated to dryness. The resulting residue was purified by Prep-HPLC (Method A) to give titled compound (45 mg, 48% for two steps). ¹H NMR (400 MHz, CD₃OD) δ 9.24-9.19 (m, 1H), 8.13-8.00 (m, 2H), 7.94-7.86 (m, 1H), 7.70-7.61 (m, 1H), 5.85-5.69 (m, 0.5H), 5.59-5.42 (m, 2,5H), 5.28-5.14 (m, 0.5H), 4.99-4.91 (m, 0.5H), 3.98-3.88 (m, 0.5H), 3.83-3.73 (m, 0.5H), 3.57-3.44 (m, 0.5H), 3.40 (s, 3H), 3.28-3.21 (m, 0.5H), 3.16-3.02 (m, 1H), 2.97-2.79 (m, 0.5H), 2.73-2,54 (m, 0.5H), 2,54-2.32 (m, 1H), 2.24-1.78 (m, 2H), 1.68-1,25 (m, 5H), 1.10-0.93 (m, 3H), 0.75-0.58 (m, 3H) ppm MS: M/e 491 (M+1)⁺.

### Compound A124: 2-(7-((2S,5R)-2,5-diethyl-4-(1-(2-methylbenzo[d]thiazol-6-yl)ethyl)piperazin-1-yl)-4-methyl-5-oxo-4,5-dihydro-2H-pyrazolo[4,3-d]pyrimidin-2

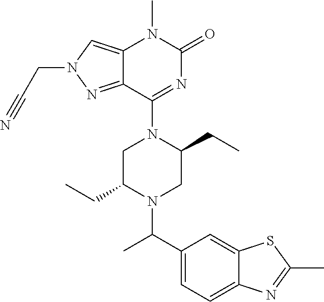 [SMILES: CC[C@H]1CN(C(C)c2ccc3nc(C)sc3c2)[C@H](CC)CN1c1nc(=O)n(C)c2cn(CC#N)nc12]

### Step A: tert-butyl (2S,5R)-2,5-diethyl-4-(1-(2-methylbenzo[d]thiazol-6-yl)ethyl)piperazine-1-carboxylate

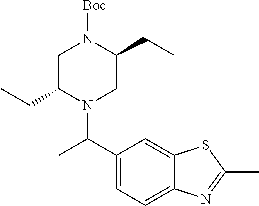 [SMILES: CC[C@H]1CN(C(C)c2ccc3nc(C)sc3c2)[C@H](CC)CN1C(=O)OC(C)(C)C]

To a solution of tert-butyl (2S,5R)-2,5-diethylpiperazine-1-carboxylate (242 mg, 1 mmol), 1-(2-methylbenzo[d]thiazol-6-yl)ethan-1-ol (200 mg, 1 mmol) and DIPEA (1,29 g, 10 mol) in CH₃CN (4 ml ) was added (cyanomethyl)trimethylphosphonium iodide (730 mg. 3 mmol). The mixture was stirred at 103° C. in a sealed bottle for 16 hours. The reaction was cooled to room temperature, diluted by adding water, extracted with EA (60 mL×2), washed with brine, dried over Na₂SO₄, filtered and concentrated to dryness. The resulting residue was purified by flash column chromatography (EA/PE=1/1) to give the titled compound (200 mg, 47%, crude). MS: M/e 418 (M+1)⁺.

### Step B: 6-(-1-((S-2S,5S)-2,5-diethylpiperazin-1-yl)ethyl)-2-methylbenzo[d]thiazole

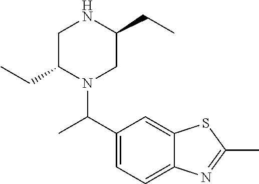 [SMILES: CC[C@H]1CN(C(C)c2ccc3nc(C)sc3c2)[C@H](CC)CN1]

To a solution of tert-butyl (2S,5R)-2,5-diethyl-4-(1-(2-methylbenzo[d]thiazol-6-yl)ethyl)piperazine-1-carboxylate (200 mg, 0.5 mmol) in DCM (5 mL) was added TMSOTf (133 mg, 0.6 mmol). The resulted mixture was stirred at room temperature for 4 h. The mixture was diluted with water (30 mL), extracted with DCM (40 mL). The aqueous layer was collected, basified with Na₂CO₃ solution to pH=9, extracted with DCM/MeOH (60 mL×3, 10/1), dried over Na₂SO₄, filtered, and concentrated to dryness. concentrated under reduced pressure, to give titled compound (30 mg). MS: M/e 318 (M+1)⁺.

### Step C: 7-((2S,5R)-2,5-diethyl-4-(1-(2-methylbenzo[d]thiazol-6-yl)ethyl)piperazin-1-yl)-4-methyl-2-(tetrahydro-2H-pyran-2-yl)-2,4-dihydro-5H-pyrazolo[4,3-d]pyrimidin-5-one

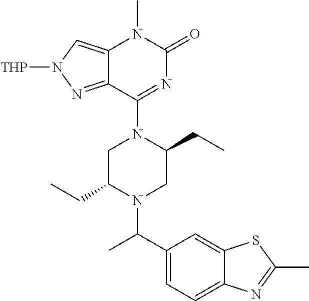

To a mixture of 4-methyl-2-(tetrahydro-2H-pyran-2-yl)-2,4-dihydro-5H-pyrazolo[4,3-d]pyrimidine-5,7 (6H)-dione (25 mg, 0.2 mmol) in CH₃CN (1 mL) was added DIPEA (50 mg, 0.4 mmol), followed by POCl₃ (54 mg, 0.4 mmol) and catalyst amount of DMF. The reaction was stirred at 85° C. for 2 hrs. The reaction was cooled to room temperature, quenched with ice-water, extracted with EtOAc (60 mL×2), washed with brine, dried over Na₂SO₄, filtered, and concentrated to dryness.

The above residue was dissolved in CH₃CN (1 mL). 6-(1-((2R,5S)-2,5-diethylpiperazin-1-yl)ethyl)-2-methylbenzo[d]thiazole (30 mg, 0.1 mmol) and DIPEA (50 mg, 0.4 mmol) were added. The resulting mixture was heated at 80° C. for 16 hrs. The reaction was quenched with water, extracted with EtOAc (60 mL×2), washed with brine, dried over Na₂SO₄, filtered, and concentrated to dryness. The resulting residue was purified by flash column chromatography (DCM/MeOH=20/1) to give titled compound (15 mg, 14%). MS: M/e 550 (M+1)⁺.

### Step D: 7-((2S,5R)-2,5-diethyl-4-(1-(2-methylbenzo[d]thiazol-6-yl)ethyl)piperazin-1-yl)-4-methyl-2,4-dihydro-5H-pyrazolo[4,3-d]pyrimidin-5-one

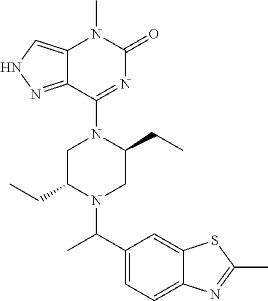 [SMILES: CC[C@H]1CN(C(C)c2ccc3nc(C)sc3c2)[C@H](CC)CN1c1nc(=O)n(C)c2c[nH]nc12]

To a solution of 7-((2S,5R)-2,5-diethyl-4-(1-(2-methylbenzo[d]thiazol-6-yl)ethyl)piperazin-1-yl)-4-methyl-2-(tetrahydro-2H-pyran-2-yl)-2,4-dihydro-5H-pyrazolo[4,3-d]pyrimidin-5-one (15 mg, 0.027 mmol) in DCM (1 mL) was added TFA (3 mL). The resulted mixture was stirred at room temperature for 16 hrs. The mixture was concentrated under reduced pressure to give titled compound (crude). MS: M/e 466 (M+1)⁺.

### Step E: 2-(7-((2S,5R)-2,5-diethyl-4-(1-(2-methylbenzo[d]thiazol-6-yl)ethyl)ioerazin-1-yl)-4-methyl-5-oxo-4,5-dihydro-2H-pyrazolo[4,3-d]pyrimidin-2-yl)acetonitrile _NYO N>

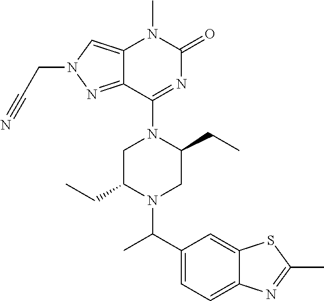 [SMILES: CC[C@H]1CN(C(C)c2ccc3nc(C)sc3c2)[C@H](CC)CN1c1nc(=O)n(C)c2cn(CC#N)nc12]

To a mixture of 7-((2S,5R)-2,5-diethyl-4-(1-(2-methylbenzo[d]thiazol-6-yl)ethyl)piperazin-1-yl)-4-methyl-2,4-dihydro-5H-pyrazolo[4,3-d]pyrimidin-5-one (16 mg, crude) and K₂CO₃ (38 mg, 0.27 mmol) in DMF (1 mL) was added 2-chloroacetonitrile (8 mg, 0.108 mmol). The reaction was stirred at room temperature for 16 hrs. The reaction was diluted with water, extracted with EtOAc (60 mL×2), washed with brine, dried over Na₂SO₄, filtered, and concentrated to dryness. The resulting residue was purified by Prep-HPLC (Method A) to give titled compound (4 mg, 29% for two steps). ¹H NMR (400 MHz, CD₃OD) δ 7.97-7.79 (m, 3H), 7.59-7.50 (m, 1H), 5.85-5.40 (m, 3H), 5.26-5.14 (m, 0.5H), 4.98-4.91 (m, 0.5H), 3.95-3.69 (m, 1H), 3.56-3.44 (m, 0.5H), 3.41 (s, 3H), 3.25-3.00 (m, 1.5H), 2.95-2.75 (m, 3.5H), 2.71-2,54 (m, 0.5H), 2,51-2.30 (m, 1H), 2.22-1.75 (m, 2H), 1.65-1,25 (m, 5H), 1.10-0.83 (m, 3H), 0.75-0.59 (m, 3H) ppm. MS: M/e 505 (M+1)⁺.

### Compound A125: 2-(7-((2S,5R)-2,5-diethyl-4-(1-(7-fluoro-2-methylbenzo[d]thiazol-6-yl)ethyl)piperazin-1-yl)-4-methyl-5-oxo-4,5-dihydro-2H-pyrazolo[4,3-d]pyrimidin-2-yl)acetonitrile

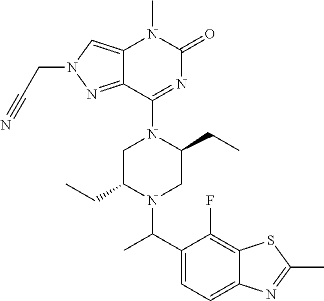 [SMILES: CC[C@H]1CN(C(C)c2ccc3nc(C)sc3c2F)[C@H](CC)CN1c1nc(=O)n(C)c2cn(CC#N)nc12]

### Step A: tert-butyl (2S,5R)-2,5-diethyl-4-(1-(7-fluoro-2-methylbenzo[d]thiazol-6-yl)ethyl)piperazine-1-carboxylate

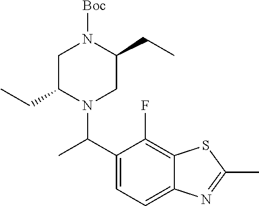 [SMILES: CC[C@H]1CN(C(C)c2ccc3nc(C)sc3c2F)[C@H](CC)CN1C(=O)OC(C)(C)C]

To a solution of tert-butyl (2S,5R)-2,5-diethylpiperazine-1-carboxylate (242 mg, 1 mmol), 1-(7-fluoro-2-methylbenzo[d]thiazol-6-yl)ethan-1-ol (400 mg, 2 mmol) and DIPEA (1,29 g, 10 mol) in CH₃CN (4 ml ) was added (cyanomethyl)trimethylphosphonium iodide (730 mg. 3 mmol). The mixture was stirred at 103° C. in a sealed bottle for 16 hours. The reaction was cooled to room temperature, diluted by adding water, extracted with EA (60 mL×2), washed with brine, dried over Na₂SO₄, filtered and concentrated to dryness. The resulting residue was purified by flash column chromatography (EA/PE=1/1) to give the titled compound (380 mg, crude). MS: M/e 436 (M+1)⁺.

### Step B: 6-(1-((2R,5S)-2,5-diethylpiperazin-1-yl)ethyl˜7-fluoro-2-methylbenzo[d]thiazole

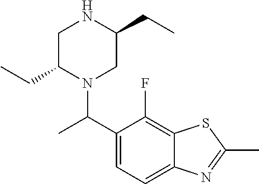 [SMILES: CC[C@H]1CN(C(C)c2ccc3nc(C)sc3c2F)[C@H](CC)CN1]

To a solution of tert-butyl (2S,5R)-2,5-diethyl-4-(1-(7-fluoro-2-methylbenzo[d]thiazol-6-yl)ethyl)piperazine-1-carboxylate (380 mg, 0.87 mmol) in DCM (5 mL) was added TMSOTf (193 mg, 0.87 mmol). The resulted mixture was stirred at room temperature for 4 hrs. The mixture was diluted with water/TFA (30 mL/1 mL), extracted with DCM (40 mL). The aqueous layer was collected, basified with Na₂CO₃ solution to pH=9, extracted with DCM/MeOH (60 mL×3, 10/1), dried over Na₂SO₄, filtered, and concentrated to dryness to give titled compound (150 mg, 44% for two steps). MS: M/e 336 (M+1)⁺.

### Step C: 7-((2S,5R)-2,5-diethyl-4-(1-(7-fluoro-2-methylbenzo[d]thiazol-6-yl)ethyl)piperazin-1-yl)-4-methyl-2-(tetrahdro-2H-pyran-2-yl)-2,4-dihydro-5H-pyrazolo[4,3-d]pyrimidin-5-one

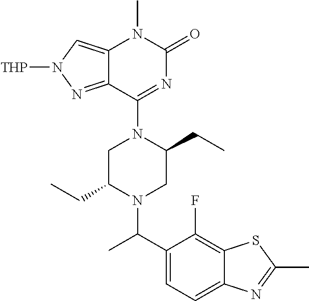

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5 mmol) in CH₃CN (3 mL) was added DIPEA (129 mg, 1 mmol), followed by POCl₃ (153 mg, 1 mmol) and catalyst amount of DMF. The reaction was stirred at 80° C. for 2 hrs. The reaction was cooled to room temperature, quenched with ice-water, extracted with EtOAc (60 mL×2), washed with brine, dried over Na₂SO₄, filtered, and concentrated to dryness.

The above residue was dissolved in CH₃CN (3 mL). 6-(1-((2R,5S)-2,5-diethylpiperazin-1-yl)ethyl)-7-fluoro-2-methylbenzo[d]thiazole (70 mg, 0.2 mmol) and DIPEA (129 mg, 1 mmol) were added. The resulting mixture was heated at 85° C. for 3 hrs. The reaction was quenched with water, extracted with EtOAc (60 mL×2), washed with brine, dried over Na₂SO₄, filtered, and concentrated to dryness. The resulting residue was purified by flash column chromatography (DCM/MeOH=20/1) to give titled compound (80 mg, 28%). MS: M/e 568 (M+1)⁺.

### Step D: 7-((2S,5R)-2,5-diethyl-4-(1-(7-fluoro-2-methylbenzo[d]thiazol-6-yl)ethyl)piperazin-1-yl)-4-methyl-2,4-dihydro-5H-pyrazolo[4,3-d]pyrimidin-5-one

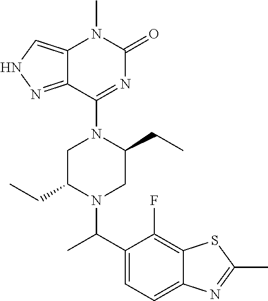 [SMILES: CC[C@H]1CN(C(C)c2ccc3nc(C)sc3c2F)[C@H](CC)CN1c1nc(=O)n(C)c2c[nH]nc12]

To a solution of 7-((2S,5R)-2,5-diethyl-4-(1-(7-fluoro-2-methylbenzo[d]thiazol-6-yl)ethyl)piperazin-1-yl)-4-methyl-2-(tetrahydro-2H-pyran-2-yl)-2,4-dihydro-5H-pyrazolo[4,3-d]pyrimidin-5-one (80 mg, 0.28 mmol) in DCM (2 mL) was added TFA (6 mL). The resulted mixture was stirred at room temperature for 16 hrs. The mixture was concentrated under reduced pressure to give titled compound (crude). MS: M/e 484 (M+1)⁺.

### Step E: 2-(7-((2S,5R)-2,5-diethyl-4-(1-(7-fluoro-2-methylbenzo[d]thiazol-6-yl)ethyl)ioerazin-1-yl)-4-methyl-5-oxo-4,5-dihydro-2H-pyrazolo[4,3-d]pyrimidin-2-yl)acetonitrile

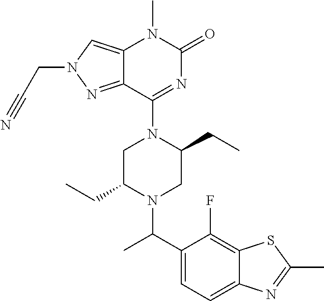 [SMILES: CC[C@H]1CN(C(C)c2ccc3nc(C)sc3c2F)[C@H](CC)CN1c1nc(=O)n(C)c2cn(CC#N)nc12]

To a mixture of 7-((2S,5R)-2,5-diethyl-4-(1-(7-fluoro-2-methylbenzo[d]thiazol-6-yl)ethyl)piperazin-1-yl)-4-methyl-2,4-dihydro-5H-pyrazolo[4,3-d]pyrimidin-5-one (80 mg, crude) and K₂CO₃ (176 mg, 1,28 mmol) in DMF (5 mL) was added 2-chloroacetonitrile (29 mg, 0.38 mmol). The reaction was stirred at room temperature for 16 hrs. The reaction was diluted with water, extracted with EtOAc (60 mL×2), washed with brine, dried over Na₂SO₄, filtered, and concentrated to dryness. The resulting residue was purified by Prep-HPLC (Method B) to give the titled Compound A125a (11 mg) and Compound A125b (15 mg).

Compound A125a (the earlier peak): ¹H NMR (400 MHz, CD₃OD) δ 7.94-7.88 (m, 1H), 7.76 (d, J=8.4 Hz, 1H), 7.73-7.64 (m, 1H), 5.77-5.64 (m, 0.5H), 5.57-5.49 (m, 0.5H), 5.24-5.12 (m, 0.5H), 5.00-4.92 (m, 0.5H), 4.91-4.83 (m, 2H), 4.34-4.22 (m, 1H), 3.54-3.44 (m, 1H), 3.41 (s, 3H), 3.15-2.87 (m, 2H), 2.87 (s, 3H), 2,50-2.32 (m, 1H), 2.20-1.90 (m, 2H), 1.69-1,24 (m, 5H), 1.02-0.90 (m, 3H), 0.73 (t, J=6.8 Hz, 1.5H), 0.67 (t, J=7.2 Hz, 1.5H) ppm. MS: M/e 523 (M+1)⁺.

Compound A125b (the later peak): ¹H NMR (400 MHz, CD₃OD) δ 7.94-7.88 (m, 1H), 7.82-7.68 (m, 2H), 5.86-5.76 (m, 0.5H), 5.54-5.42 (m, 0.5H), 5.28-5.18 (m, 0.5H), 5.02-4.91 (m, 0.5H), 4.91-4.83 (m, 2H), 4.22-4.08 (m, 1H), 3.80-3.70 (m, 0.5H), 3.41 (s, 3H), 3.28-3.16 (m, 1H), 2.85 (s, 3H), 2.76-2,57 (m, 1H), 2.39-2.28 (m, 1H), 2.05-1.74 (m, 2H), 1.64-1.45 (m, 2H), 1.39 (d, J=6.0 Hz, 3H), 1.11-0.92 (m, 3H), 0.72-0.60 (m, 3H) ppm. MS: M/e 523 (M+1)⁺.

### Compound A126: 2-(7-((2S,5R)-2,5-diethyl-4-(1-(thiazolo[5,4-b]pyridin-5-yl)ethyl)piperazin-1-yl)-4-methyl-5-oxo-4,5-dihydro-2H-pyrazolo[4,3-d]pyrimidin-2-yl)acetonitrile

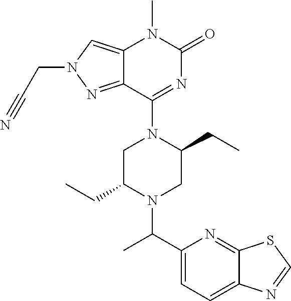 [SMILES: CC[C@H]1CN(C(C)c2ccc3ncsc3n2)[C@H](CC)CN1c1nc(=O)n(C)c2cn(CC#N)nc12]

### Step A: tert-butyl (2S,5R)-2,5-diethyl)-4-(14thiazolo[5.4-b]pyridin-5-yl)ethyl)piperazine-1-carboxylate

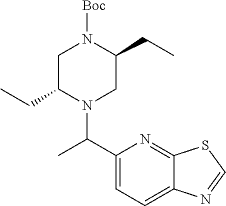 [SMILES: CC[C@H]1CN(C(C)c2ccc3ncsc3n2)[C@H](CC)CN1C(=O)OC(C)(C)C]

To a solution of tert-butyl (2S,5R)-2,5-diethylpiperazine-1-carboxylate (242 mg, 1 mmol), 1-(thiazolo[5,4-b]pyridin-5-yl)ethan-1-ol (360 mg, 2 mmol) and DIPEA (1,29 g, 10 mol) in CH₃CN (5 ml ) was added (cyanomethyl)trimethylphosphonium iodide (730 mg. 3 mmol). The mixture was stirred at 103° C. in a sealed bottle for 16 hours. The reaction was cooled to room temperature, diluted by adding water, extracted with EA (60 mL×2), washed with brine, dried over Na₂SO₄, filtered and concentrated to dryness. The resulting residue was purified by flash column chromatography (EA/PE=1/1) to give the titled compound (365 mg, crude). MS: M/e 405 (M+1)⁺.

### Step B: 5-(1-((2R,5S)-2,5-diethylpiperazin-1-yl)ethyl)thiazolo[5.4-b]pyridine

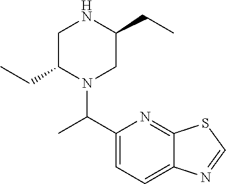 [SMILES: CC[C@H]1CN(C(C)c2ccc3ncsc3n2)[C@H](CC)CN1]

To a solution of tert-butyl (2S,5R)-2,5-diethyl-4-(1-(thiazolo[5,4-b]pyridin-5-yl)ethyl)piperazine-1-carboxylate (365 mg, 0.90 mmol) in DCM (5 mL) was added TMSOTf (200 mg, 0.9 mmol). The resulted mixture was stirred at room temperature for 4 hrs. The mixture was diluted with water/TFA (30 mL/1 mL), extracted with DCM (40 mL). The aqueous layer was collected, basified with Na₂CO₃ solution to pH=9, extracted with DCM/MeOH (60 mL×3, 10/1), dried over Na₂SO₄, filtered, and concentrated to dryness. concentrated under reduced pressure, to give titled compound (240 mg, 75% for two steps). MS: M/e 305 (M+1)⁺.

### Step C: 7-((2S,5R)-2,5-diethyl-4-(1-(thiazolo[5.4-b]pyridin-5-yl)ethyl)piperazin-1-yl-4-methyl-2-(tetrahydro-2H-pyran-2-yl)-2,4-dihydro-5H-pyrazolo[4,3-d]pyrimidin-5-one

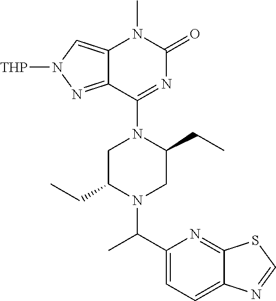

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5 mmol) in CH₃CN (3 mL) was added DIPEA (129 mg, 1 mmol), followed by POCl₃ (153 mg, 1 mmol) and catalyst amount of DMF. The reaction was stirred at 80° C. for 2 h. The reaction was cooled to room temperature, quenched with ice-water, extracted with EtOAc (60 mL×2), washed with brine, dried over Na₂SO₄, filtered, and concentrated to dryness.

The above residue was dissolved in CH₃CN (3 mL). 5-(1-((2R,5S)-2,5-diethylpiperazin-1-yl)ethyl)thiazolo[5,4-b]pyridine (121 mg, 0.5 mmol) and DIPEA (129 mg, 1 mmol) were added. The resulting mixture was heated at 85° C. for 16 hrs. The reaction was quenched with water, extracted with EtOAc (60 mL×2), washed with brine, dried over Na₂SO₄, filtered, and concentrated to dryness. The resulting residue was purified by flash column chromatography (DCM/MeOH=20/1) to give titled compound (70 mg, 26%). MS: M/e 537 (M+1)⁺.

### Step D: 7-((2S,5R)-2,5-diethyl-4-(1-(thiazolo[5.4-b]pyridin-5-yl)ethyl)lpiperazin-1-yl)-4-methyl-2,4-dihydro-5H-pyrazolo[4,3-d]pyrimidin-5-one

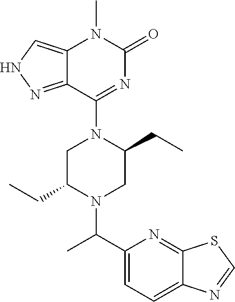 [SMILES: CC[C@H]1CN(C(C)c2ccc3ncsc3n2)[C@H](CC)CN1c1nc(=O)n(C)c2c[nH]nc12]

To a solution of 7-((2S,5R)-2,5-diethyl-4-(1-(thiazolo[5,4-b]pyridin-5-yl)ethyl)piperazin-1-yl)-4-methyl-2-(tetrahydro-2H-pyran-2-yl)-2,4-dihydro-5H-pyrazolo[4,3-d]pyrimidin-5-one (70 mg, 0.13 mmol) in DCM (2 mL) was added TFA (6 mL). The resulted mixture was stirred at room temperature for 16 hrs. The mixture was concentrated under reduced pressure to give titled compound (crude). MS: M/e 453 (M+1)⁺.

### Step E: 2-(7-((2S,5R)-2,5-diethyl-4-(1-(thiazolo[5.4-b]pyridin-5-yl)ethyl)piperazin-1-yl)-4-methyl-5-oxo-4,5-dihydro-2H-pyrazolo[4,3-d]pyrimidin-2-yl)acetonitrile

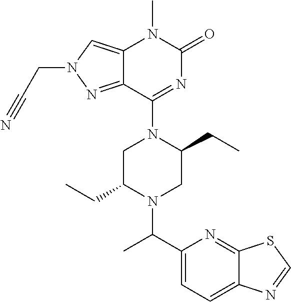 [SMILES: CC[C@H]1CN(C(C)c2ccc3ncsc3n2)[C@H](CC)CN1c1nc(=O)n(C)c2cn(CC#N)nc12]

To a mixture of 7-((2S,5R)-2,5-diethyl-4-(1-(thiazolo[5,4-b]pyridin-5-yl)ethyl)piperazin-1-yl)-4-methyl-2,4-dihydro-5H-pyrazolo[4,3-d]pyrimidin-5-one (70 mg, crude) and K₂CO₃ (180 mg, 1.3 mmol) in DMF (5 mL) was added 2-chloroacetonitrile (39 mg, 0.52 mmol). The reaction was stirred at room temperature for 16 hrs. The reaction was diluted with water, extracted with EtOAc (60 mL×2), washed with brine, dried over Na₂SO₄, filtered, and concentrated to dryness. The resulting residue was purified by Prep-HPLC (Method A) to give titled Compound A126a (13 mg) and Compound A126b (16 mg).

Compound A126a (the earlier peak): ¹H NMR (400 MHz, CD₃OD) δ 9.35 (s, 1H), 8.43 (d, J=8.8 Hz, 1H), 7.92 (d, J=9.6 Hz, 1H), 7.86 (d, J=8.4 Hz, 1H), 5.86-5.79 (m, 0.5H), 5.61-5.44 (m, 2,5H), 5.29-5.20 (m, 0.5H), 5.03-4.92 (m, 0.5H), 4.00-3.88 (m, 1H), 3.84-3.76 (m, 0.5H), 3.41 (s, 3H), 3.40-3.33 (m, 0.5H), 3.29-3.15 (m, 11H), 2.86-2.64 (m, 1H), 2.34-2.22 (m, 1H), 2.06-1.74 (m, 2H), 1.62-1.47 (m, 2H), 1.42 (d, J=6.4 Hz, 3H), 1.13-0.95 (m, 3H), 0.68 (t, J=7.2 Hz, 3H) ppm. MS: M/e 492 (M+1)⁺.

Compound A126b (the later peak): ¹H NMR (400 MHz, CD₃OD) δ 9.35 (s, 1H), 8.44 (d, J=8.4 Hz, 1H), 7.96-7.78 (m, 2H), 5.79-5.65 (m, 0.5H), 5.60-5.44 (m, 2,5H), 5.24-5.14 (m, 0.5H), 5.02-4.94 (m, 0.5H), 4.15-4.05 (m, 1H), 3.64-3.42 (m, 0.5H), 3.41 (s, 3H), 3.22-2.80 (m, 2,5H), 2,47-2.35 (m, 1H), 2.20-1.95 (m, 2H), 1.70-1.32 (m, 5H), 1.02-0.90 (m, 3H), 0.74 (t, J=7.6 Hz, 1.5H), 0.68 (t, J=7.6 Hz, 1.5H) ppm. MS: We 492 (M+1)⁺.

### Compound A127: 2-(7-((2S,5R)-4-(1-(6-cyclopropylpyridin-3-yl)ethyl)-2,5-diethylpiperazin-1-yl)-4-methyl-5-oxo-4,5-dihydro-2H-pyrazolo[4,3-d]pyrimidin-2-yl)acetonitrile

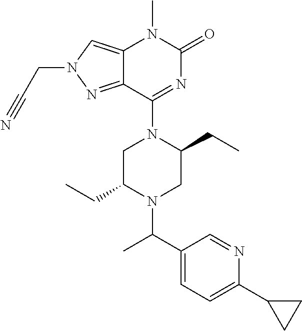 [SMILES: CC[C@H]1CN(C(C)c2ccc(C3CC3)nc2)[C@H](CC)CN1c1nc(=O)n(C)c2cn(CC#N)nc12]

### Step A: tert-butyl (2S,5R)₄-(1-(6-cycloaroovlpyridin-3-yl)ethyl)˜2,5-diethylpiperazine-1-carboxylate

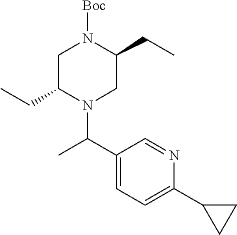 [SMILES: CC[C@H]1CN(C(C)c2ccc(C3CC3)nc2)[C@H](CC)CN1C(=O)OC(C)(C)C]

To a solution of tert-butyl (2S,5R)-2,5-diethylpiperazine-1-carboxylate (120 mg, 0.5 mmol), 1-(6-cyclopropylpyridin-3-yl)ethan-1-ol (163 mg, 1 mmol) and DIPEA (645 mg, 5 mol) in CH₃CN (2 ml ) was added (cyanomethyl)trimethylphosphonium iodide (365 mg, 3 mmol). The mixture was stirred at 103° C. in a sealed bottle for 16 hours. The reaction was cooled to room temperature, diluted by adding water, extracted with EA (60 mL×2), washed with brine, dried over Na₂SO₄, filtered and concentrated to dryness. The resulting residue was purified by flash column chromatography (EA/PE=1/1) to give the titled compound (60 mg, 31%). MS: M/e 388 (M+1)⁺.

### Step B: (2R,5S)-1-(1-(6-cyclopropylpyridin-3-yl)ethyl)-2,5-diethylpiperazine

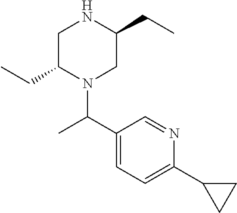 [SMILES: CC[C@H]1CN(C(C)c2ccc(C3CC3)nc2)[C@H](CC)CN1]

To a solution of tert-butyl (2S,5R)-4-(1-(6-cyclopropylpyridin-3-yl)ethyl)-2,5-diethylpiperazine-1-carboxylate (60 mg, 0.155 mmol) in DCM (2 mL) was added TFA (0.5 mL). The resulted mixture was stirred at room temperature for 16 hrs. The mixture was concentrated under reduced pressure to give titled compound (60 mg, crude). MS: M/e 288 (M+1)⁺.

### Step C: 7-((2S,5R)-4-(1-(6-cyclopropylpyridin-3-yl)ethyl)-2,5-diethylpiperazin-1-yl-4-methyl-2-(tetrahydro-2H-pyran-2-yl)-2,4-dihydro-5H-pyrazoloI43-d]pyrimidin-5-one

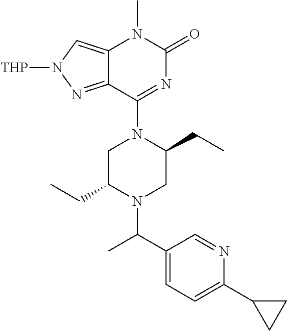

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5 mmol) in CH₃CN (3 mL) was added DIPEA (129 mg, 1 mmol), followed by POCl₃ (153 mg, 1 mmol) and catalyst amount of DMF. The reaction was stirred at 80° C. for 2 hrs. The reaction was cooled to room temperature, quenched with ice-water, extracted with EtOAc (60 mL×2), washed with brine, dried over Na₂SO₄, filtered, and concentrated to dryness.

The above residue was dissolved in CH₃CN (3 mL). (2R,5S)-1-(1-(6-cyclopropylpyridin-3-yl)ethyl)-2,5-diethylpiperazine (60 mg, crude) and DIPEA (129 mg, 1 mmol) were added. The resulting mixture was heated at 85° C. for 16 hrs. The reaction was quenched with water, extracted with EtOAc (60 mL×2), washed with brine, dried over Na₂SO₄, filtered, and concentrated to dryness. The resulting residue was purified by flash column chromatography (D/M=20/1) to give titled compound (85 mg, 32%). MS: M/e 520 (M+1)⁺.

### Step D: 7-((2S,5R)₄-(1-(6-ccloprovlpyridin-3-yl)ethyl-2,5-diethylpiperazin-1-yl)₄-methyl-2,4-dihydro-5H-pyrazolo[4,3-d]pyrimidin-5-one

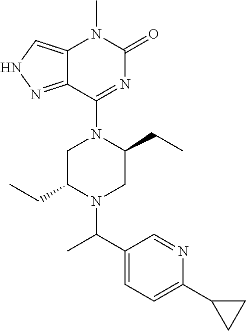 [SMILES: CC[C@H]1CN(C(C)c2ccc(C3CC3)nc2)[C@H](CC)CN1c1nc(=O)n(C)c2c[nH]nc12]

To a solution of 7-((2S,5R)-4-(1-(6-cyclopropylpyridin-3-yl)ethyl)-2,5-diethylpiperazin-1-yl)-4-methyl-2-(tetrahydro-2H-pyran-2-yl)-2,4-dihydro-5H-pyrazolo[4,3-d]pyrimidin-5-one (85 mg, 0.163 mmol) in DCM (2 mL) was added TFA (6 mL). The resulted mixture was stirred at room temperature for 16 hrs. The mixture was concentrated under reduced pressure to give titled compound (85 mg crude). MS: M/e 436 (M+1)⁺.

### Step E: 2-(7-((2S,5R)-4-(1-(6-cyclonroovlpyridin-3-yl)ethyl)-2,5-diethylpiperazin-1-yl-4-methyl-5-oxo-4,5-dihydro-2H-pyrazolo[4,3-d]pyrimidin-2-yl)acetonitrile

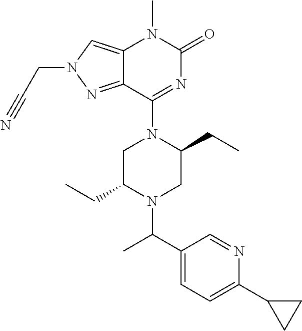 [SMILES: CC[C@H]1CN(C(C)c2ccc(C3CC3)nc2)[C@H](CC)CN1c1nc(=O)n(C)c2cn(CC#N)nc12]

To a mixture of 7-((2S,5R)-4-(1-(6-cyclopropylpyridin-3-yl)ethyl)-2,5-diethylpiperazin-1-yl)-4-methyl-2,4-dihydro-5H-pyrazolo[4,3-d]pyrimidin-5-one (85 mg, crude) and K₂CO₃ (225 mg, 1.63 mmol) in DMF (3 mL) was added 2-chloroacetonitrile (37 mg, 0.48 mmol). The reaction was stirred at room temperature for 16 hrs. The reaction was diluted with water, extracted with EtOAc (60 mL×2), washed with brine, dried over Na₂SO₄, filtered, and concentrated to dryness. The resulting residue was purified by Prep-HPLC (Method A) to give the titled Compound A127a (11 mg) and Compound A127b (15 mg).

Compound A127a (the earlier peak): ¹H NMR (400 MHz, CD₃OD) δ 8.32-8.28 (m, 1H), 7.91 (d, J=9.6 Hz, 1H), 7.75-7.69 (m, 1H), 7.21 (d, J=8.0 Hz, 1H), 5.76-5.66 (m, 0.5H), 5.58-5.44 (m, 2,5H), 5.22-5.12 (m, 0.5H), 5.03-4.92 (m, 0.5H), 3.82-3.70 (m, 1H), 3.53-3.46 (m, 0.5H), 3.40 (s, 3H), 3.18-2.75 (m, 2,5H), 2,45-2.35 (m, 1H), 2.15-1.95 (m, 3H), 1.56-1,27 (m, 5H), 1.10-0.88 (m, 7H), 0.78-0.63 (m, 3H) ppm. MS: M/e 475 (M+1)⁺.

Compound A127b (the later peak): ¹H NMR (400 MHz, CD₃OD) δ 8.34 (s, 1H), 7.91 (d, J=6.4 Hz, 1H), 7.78-7.70 (m, 1H), 7.19 (d, J=8.4 Hz, 1H), 5.86-5.76 (m, 0.5H), 5.58-5.44 (m, 2,5H), 5.31-5.20 (m, 0.5H), 5.02-4.94 (m, 0.5H), 3.76-3.58 (m, 1.5H), 3.41 (s, 3H), 3.28-3.12 (m, 1.5H), 2.74-2,53 (m, 1H), 2.37-2.26 (m, 1H), 2.16-2.02 (m, 1H), 1.99-1.70 (m, 2H), 1.59-1.40 (m, 2H), 1.32 (d, J=6.4 Hz, 3H), 1.09-0.89 (m, 7H), 0.68 (t, J=7.2 Hz, 3H) ppm. MS: M/e 475 (M+1)⁺.

CompoundA128: 2-(7-((2S,5R)-2,5-diethyl-4-(1-(5-fluoro-2-methylbenzo[d]thiazol-6-yl)ethyl)piperazin-1-yl)-4-methyl-5-oxo-4,5-dihydro-2H-pyrazolo[4,3-d]pyrimidin-2-yl)acetonitrile

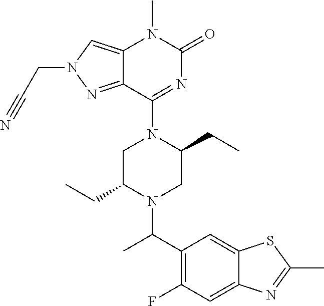 [SMILES: CC[C@H]1CN(C(C)c2cc3sc(C)nc3cc2F)[C@H](CC)CN1c1nc(=O)n(C)c2cn(CC#N)nc12]

### Step A: tert-butyl (2S,5R)-2,5-diethyl-4-fI4-5-fluoro-2-methylbenzo[d]thiazol-6-yl)ethyl)piperazine-1-carboxylate

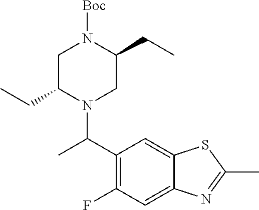 [SMILES: CC[C@H]1CN(C(C)c2cc3sc(C)nc3cc2F)[C@H](CC)CN1C(=O)OC(C)(C)C]

To a solution of tert-butyl (2S,5R)-2,5-diethylpiperazine-1-carboxylate (120 ing, 0.5 mminol), 1-(5-fluoro-2-methylbenzo[d]thiazol-6-yl)ethan-1-ol (211 mg, 1 mmol) and DIPEA (645 ing, 5 mol) in CH₃CN (2 ml ) was added (cyanomethyl)trimethylphosphonium iodide (365 ing, 3 minol). The mixture was stirred at 103° C. in a sealed bottle for 16 hours. The reaction was cooled to room temperature, diluted by adding water, extracted with EA (60 mL×2), washed with brine, dried over Na₂SO₄, filtered and concentrated to dryness. The resulting residue was purified by flash column chromatography (EA/PE=1/1) to give the titled compound (130 mg, crude). MS: M/e 436 (M+1)⁺.

### Step B: 6-1-((2R,5S)-2,5-diethylpiperazin-1-viethyl)-5-fluoro-2-methylbenzo[d]thiazole

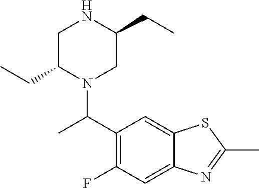 [SMILES: CC[C@H]1CN(C(C)c2cc3sc(C)nc3cc2F)[C@H](CC)CN1]

To a solution of tert-butyl (2S,5R)-2,5-diethyl-4-(1-(5-fluoro-2-methylbenzo[d]thiazol-6-yl)ethyl)piperazine-1-carboxylate (130 mg, 0.3 mmol) in DCM (5 mL) was added TMSOTf (100 mg, 0.45 mmol). The resulted mixture was stirred at room temperature for 16 hrs. The mixture was diluted with water/TFA (30 mL/1 mL), extracted with DCM (40 mL). The aqueous layer was collected, basified with Na₂CO₃ solution to pH=9, extracted with DCM/MeOH (60 mL×3, 10/1), dried over Na₂SO₄, filtered, and concentrated under reduced pressure to give titled compound (80 mg, 47% for two steps). MS: M/e 336 (M+1)⁺.

### Step C: 7-((2S,5R)-2,5-diethyl-4-(1-(5-fluoro-2-methylbenzo[d]thiazol-6-yl)ethyl)ioerazin-1-yl-4-methyl-2-(tetrahydro-2H-pyran-2-yl)-2,4-dihydro-5H-pyrazolo[4,3-d]pyrimidin-5-one

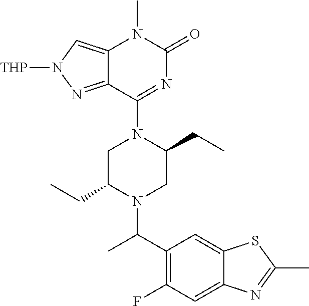

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5 mmol) in CH₃CN (3 mL) was added DIPEA (129 mg, 1 mmol), followed by POCl₃ (153 mg, 1 mmol) and catalyst amount of DMF. The reaction was stirred at 80° C. for 2 hrs. The reaction was cooled to room temperature, quenched with ice-water, extracted with EtOAc (60 mL×2), washed with brine, dried over Na₂SO₄, filtered, and concentrated to dryness.

The above residue was dissolved in CH₃CN (3 mL). 6-(1-((2R,5S)-2,5-diethylpiperazin-1-yl)ethyl)-5-fluoro-2-methylbenzo[d]thiazole (80 mg, 0.238 mmol) and DIPEA (129 mg, 1 mmol) were added. The resulting mixture was heated at 85° C. for 16 hrs. The reaction was quenched with water, extracted with EtOAc (60 mL×2), washed with brine, dried over Na₂SO₄, filtered, and concentrated to dryness. The resulting residue was purified by flash column chromatography (DCM/MeOH=20/1) to give titled compound (30 mg, 50%). MS: M/e 568 (M+1)⁺.

### Step D: 7-((2S,5R-2,5-diethyl-4-(1-(5-fluoro-2-methylbenzo[d]thiazol-6-yl)ethyl)piperazin-1-yl)-4-methyl-2,4-dihydro-5H-pyrazolo[4,3-d]pyrimidin-5-one

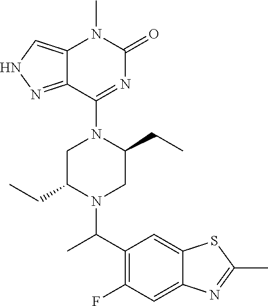 [SMILES: CC[C@H]1CN(C(C)c2cc3sc(C)nc3cc2F)[C@H](CC)CN1c1nc(=O)n(C)c2c[nH]nc12]

To a solution of 7-((2S,5R)-2,5-diethyl-4-(1-(5-fluoro-2-methylbenzo[d]thiazol-6-yl)ethyl)piperazin-1-yl)-4-methyl-2-(tetrahydro-2H-pyran-2-yl)-2,4-dihydro-5H-pyrazolo[4,3-d]pyrimidin-5-one (30 mg, 0.053 mmol) in DCM (1 mL) was added TFA (3 mL). The resulted mixture was stirred at room temperature for 16 hrs. The mixture was concentrated under reduced pressure to give titled compound (crude). MS: M/e 484 (M+1)⁺.

### Step E: 2-(7-((2S,5R)-2,5-diethyl-4-(1-(5-fluoro-2-methylbenzo[d]thiazol-6-yl)ethyl)piperazin-1-yl)-4-methyl-5-oxo-4,5-dihydro-2H-pyrazolo[4,3-d]pyrimidin-2-yl)acetonitrile

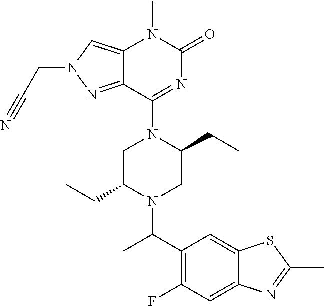 [SMILES: CC[C@H]1CN(C(C)c2cc3sc(C)nc3cc2F)[C@H](CC)CN1c1nc(=O)n(C)c2cn(CC#N)nc12]

To a mixture of 7-((2S,5R)-2,5-diethyl-4-(1-(5-fluoro-2-methylbenzo[d]thiazol-6-yl)ethyl)piperazin-1-yl)-4-methyl-2,4-dihydro-5H-pyrazolo[4,3-d]pyrimidin-5-one (30 mg, crude) and K₂CO₃ (73 mg, 0.53 mmol) in DMF (2 mL) was added 2-chloroacetonitrile (12 mg, 0.16 mmol). The reaction was stirred at room temperature for 16 hrs. The reaction was diluted with water, extracted with EtOAc (60 mL×2), washed with brine, dried over Na₂SO₄, filtered, and concentrated to dryness. The resulting residue was purified by Prep-HPLC (Method A) to give the titled Compound 128a (5 mg) and Compound 128b (7 mg).

Compound A128a (the earlier peak): ¹H NMR (400 MHz, CD₃OD) δ 8.14-8.06 (m, 1H), 7.92 (d, J=10.8 Hz, 1H), 7.59 (d, J=10.4 Hz, 1H), 5.76-5.66 (m, 0.5H), 5.58-5.44 (m, 2,5H), 5.22-5.12 (m, 0.5H), 5.03-4.92 (m, 0.5H), 4.35-4.18 (m, 1H), 3.58-3.46 (m, 0.5H), 3.40 (s, 3H), 3.40-3.33 (m, 0.5H), 3.16-2.90 (m, 2H), 2.84 (s, 3H), 2,58-2,44 (m, 1H), 2.20-1.86 (m, 2H), 1.65-1.32 (m, 5H), 0.99-0.88 (m, 3H), 0.82-0.68 (m, 3H) ppm. MS: M/e 523 (M+1)⁺.

Compound A128b (the later peak): ¹H NMR (400 MHz, CD₃OD) δ 8.19 (s, 1H), 7.92 (d, J=9.2 Hz, 1H), 7.57 (d, J=10.4 Hz, 1H), 5.86-5.76 (m, 0.5H), 5.58-5.44 (m, 2,5H), 5.31-5.20 (m, 0.5H), 5.02-4.94 (m, 0.5H), 4.16-4.05 (m, 1H), 3.82-3.70 (m, 1H), 3.41 (s, 3H), 3.28-3.12 (m, 1H), 2.83 (s, 3H), 2.78-2,59 (m, 1H), 2,46-2.34 (m, 1H), 2.02-1.75 (m, 2H), 1.61-1.45 (m, 2H), 1.38 (d, J=6.0 Hz, 3H), 1.12-0.96 (m, 3H), 0.67 (t, J=6.4 Hz, 3H) ppm. MS: M/e 523 (M+1)⁺.

### Compound A129: 2-(7-((2S,5R)-2,5-diethyl-4-(1-(2-methylthiazolo[5,4-b]pyridin-5-yl)ethyl)piperazin-1-yl)-4-methyl-5-oxo-4,5-dihydro-2H-pyrazolo[4,3-d]pyrimidin-2-yl)acetonitrile

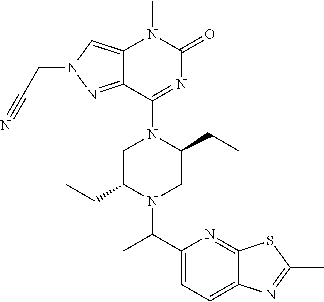 [SMILES: CC[C@H]1CN(C(C)c2ccc3nc(C)sc3n2)[C@H](CC)CN1c1nc(=O)n(C)c2cn(CC#N)nc12]

### Step A: 1-42-methylthiazolo[5.4-b]pyridin-5-yl)ethan-1-one

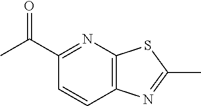

A mixture of 5-chloro-2-methylthiazolo[5,4-b]pyridine (250 mg, 1.35 mmol), tributyl(1-ethoxyvinyl)stannane (738 mg, 2.04 mmol) and Pd(PPh₃)₂Cl₂ (95 mg, 0.14 mmol) in DMF (3 mL) was stirred at 100° C. under N₂ for 16 hours. The solution was diluted with ethyl acetate and washed with water. The organic layer was purified by flash column chromatography. The product was diluted with ethyl acetate and added with HCl (3 mL, 3 M in dioxane) in drops and the mixture was stirred at room temperature for 2 hours. The reaction was concentrated and adjusted to pH=8-9 with saturated NaHCO₃ solution, extracted with ethyl acetate. The combined organic layers were washed with brine, dried over Na₂SO₄. The resulting residue was purified by flash column chromatography to give the titled compound (200 mg, 77%). MS: M/e 193 (M+1)+

### Step B: 1-(2-methylthiazolo[5.4-b]pyridin-5-yl)ethan-1-ol

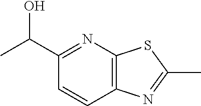

To a solution of 1-(2-methylthiazolo[5,4-b]pyridin-5-yl)ethan-1-one (200 mg, 1.04 mmol) in MeOH (3 mL) was added NaBH₄ (39 mg, 1.04 mmol) at room temperature and the resulting mixture was stirred at room temperature for 5 min. The reaction mixture was diluted with DCM and washed with water, dried over Na₂SO₄. The resulting residue was purified by flash column chromatography to give the titled compound (180 mg, 90%). MS: M/e 195 (M+1)⁺.

### Step C: tert-butyl (2S,5R)-2,5-diethyl-4-(1-(2-methylthiazolo[5.4-b]pyridin-5-yl)ethyl)piperazine-1-carboxylate

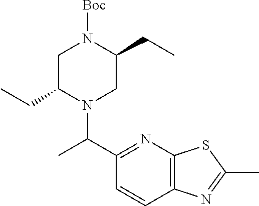 [SMILES: CC[C@H]1CN(C(C)c2ccc3nc(C)sc3n2)[C@H](CC)CN1C(=O)OC(C)(C)C]

To a solution of tert-butyl (2S,5R)-2,5-diethylpiperazine-1-carboxylate (242 mg, 1.0 mmol), 1-(2-methylthiazolo[5,4-b]pyridin-5-yl)ethan-1-ol (291 mg, 1.5 mmol) and (cyanomethyl)trimethylphosphonium iodide (730 mg, 3.0 mmol) in CH₃CN (2,5 mL) was added DIPEA (650 mg, 5.0 mmol). The mixture was stirred at 100° C. in a sealed tube for 16 hours. The mixture was cooled and diluted with EtOAc (20 mL), washed with brine (10 mL×3), dried over Na₂SO₄, filtered and concentrated under reduced pressure. The resulting residue was purified by flash column chromatography to give the titled compound (370 mg, yield: 88%). MS: M/e 419 (M+1)⁺.

### Step D: 5-(1-((2R,5S)-2,5-diethylpiperazin-1-yl)ethyl)-2-methylthiazolo[5.4-b]pyridine

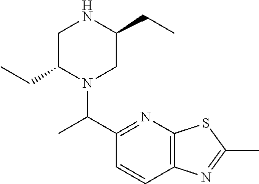 [SMILES: CC[C@H]1CN(C(C)c2ccc3nc(C)sc3n2)[C@H](CC)CN1]

To a stirred solution of tert-butyl (2S,5R)-2,5-diethyl-4-(1-(2-methylthiazolo[5,4-b]pyridin-5-yl)ethyl)piperazine-1-carboxylate (360 mg, 0.86 mmol) in MeOH (5 mL) was added HCl/Dioxane (4 M, 3 mL) at room temperature and the mixture was stirred for 3 hours. The mixture was concentrated to dryness to give the titled compound (385 mg, crude). MS: M/e 319 (M+1)⁺.

### Step E: 7-((2S,5R)-2,5-diethyl-4-(1-(2-methylthiazolo[5.4-b]pyridin-5-yl)ethyl)piperazin-1-yl-4-methyl-2-(tetrahydro-2H-pyran-2-yl)-2,4-dihydro-5H-pyrazolo[4,3-drimidin-5-one UN; Q

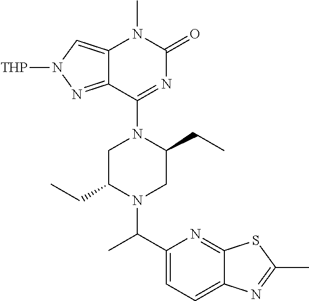

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5 mmol) in CH₃CN (3 mL) was added DIPEA (129 mg, 1 mmol), followed by POCl₃ (153 mg, 1 mmol) and catalyst amount of DMF. The reaction was stirred at 80° C. for 2 hrs. The reaction was cooled to room temperature, quenched with ice-water, extracted with EtOAc (60 mL×2), washed with brine, dried over Na₂SO₄, filtered, and concentrated to dryness.

The above residue was dissolved in CH₃CN (3 mL). 5-(1-((2R,5S)-2,5-diethylpiperazin-1-yl)ethyl)-2-methylthiazolo[5,4-b]pyridine (100 mg, 0.3 mmol) and DIPEA (129 mg, 1 mmol) were added. The resulting mixture was heated at 85° C. for 16 hrs. The reaction was quenched with water, extracted with EtOAc (60 mL×2), washed with brine, dried over Na₂SO₄, filtered, and concentrated to dryness. The resulting residue was purified by flash column chromatography (DCM/MeOH=20/1) to give titled compound (55 mg, 33%). MS: M/e 551 (M+1).

### Step F: 7-((2S,5R)-2,5-diethyl-4-(1-(2-methylthiazolo[5.4-b]pyridin-5-yl)ethyl)piperazin-1-yl)-4-methyl-2,4-dihydro-5H-pyrazolo[4,3-d]pyrimidin-5-one

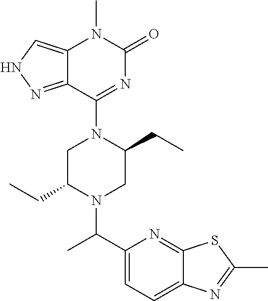 [SMILES: CC[C@H]1CN(C(C)c2ccc3nc(C)sc3n2)[C@H](CC)CN1c1nc(=O)n(C)c2c[nH]nc12]

To a solution of 7-((2S,5R)-2,5-diethyl-4-(1-(2-methylthiazolo[5,4-b]pyridin-5-yl)ethyl)piperazin-1-yl)-4-methyl-2-(tetrahydro-2H-pyran-2-yl)-2,4-dihydro-5H-pyrazolo[4,3-d]pyrimidin-5-one (55 mg, 0.1 mmol) in DCM (1 mL) was added TFA (3 mL). The resulted mixture was stirred at room temperature for 16 hrs. The mixture was concentrated under reduced pressure to give titled compound (crude). MS: M/e 467 (M+1)⁺.

### Step G: 2-(7-((2S,5R)-2,5-diethyl-4-(1-(2-methylthiazolo[5.4-b]pyridin-5-yl)ethyl)piperazin-1-yl)-4-methyl-5-oxo-4,5-dihydro-2H-pyrazolo[4,3-d]pyrimidin-2-yl)acetonitrile

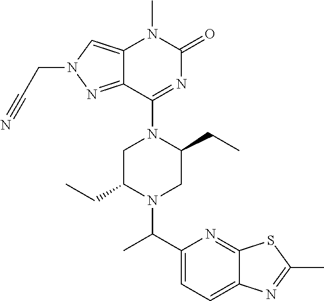 [SMILES: CC[C@H]1CN(C(C)c2ccc3nc(C)sc3n2)[C@H](CC)CN1c1nc(=O)n(C)c2cn(CC#N)nc12]

To a mixture of 7-((2S,5R)-2,5-diethyl-4-(1-(2-methylthiazolo[5,4-b]pyridin-5-yl)ethyl)piperazin-1-yl)-4-methyl-2,4-dihydro-5H-pyrazolo[4,3-d]pyrimidin-5-one (30 mg, crude) and K₂CO₃ (138 mg, 1 mmol) in DMF (2 mL) was added 2-chloroacetonitrile (15 mg, 0.2 mmol). The reaction was stirred at room temperature for 16 hrs. The reaction was diluted with water, extracted with EtOAc (80 mL), washed with brine, dried over Na₂SO₄, filtered, and concentrated to dryness. The resulting residue was purified by Prep-HPLC (Method A) to give the titled Compound 129a (8 mg) and Compound 129b (8 mg).

Compound A129a (the earlier peak): ¹H NMR (400 MHz, CD₃OD) δ 8.23 (d, J=7.6 Hz, 1H), 7.92 (d, J=9.2 Hz, 1H), 7.73 (dd, J=3.2 Hz, 8.4 Hz, 1H), 5.83-5.75 (m, 0.5H), 5.57-5.44 (m, 2,5H), 5.28-5.16 (m, 0.5H), 5.03-4.92 (m, 0.5H), 3.95-3.72 (m, 1.5H), 3.41 (s, 3H), 3.30-3.10 (m, 1.5H), 2.86 (s, 3H), 2.84-2.76 (m, 0.5H), 2.74-2.67 (m, 0.5H), 2.35-2.21 (m, 1H), 2.0.5-1.76 (m, 2H), 1.60-1.46 (m, 2H), 1.40 (d, J=6.8 Hz, 3H), 1.10-0.95 (m, 3H), 0.74-0.64 (m, 3H) ppm. MS: M/e 506 (M+1)⁺.

Compound A129b (the later peak): ¹H NMR (400 MHz, CD₃OD) δ 8.23 (d, J=8.0 Hz, 1H), 7.92 (d, J=12,4 Hz, 1H), 7.73 (dd, J=5.2 Hz, 8.8 Hz, 1H), 5.78-5.66 (m, 0.5H), 5.58-5.44 (m, 2,5H), 5.31-5.20 (m, 0.5H), 5.02-4.94 (m, 0.5H), 4.11-4.04 (m, 1H), 3.59-3.51 (m, 0.5H), 3.41 (s, 3H), 3.18-2.94 (m, 2H), 2.91-2.87 (m, 0.5H), 2.86 (s, 3H), 2,48-2.35 (m, 1H), 2.20-1.93 (m, 2H), 1.69-1.30 (m, 5H), 0.99-0.90 (m, 3H), 0.78-0.64 (m, 3H) ppm. MS: M/e 506 (M+1)⁺.

### Compound A130: 2-(7-((2S,5R)-5-ethyl-2-methyl-4-(1-(thieno[2,3-bipyridin-6-yl)ethyl)piperazin-1-yl)-4-methyl-5-oxo-4,5-dihydro-2H-pyrazolo[4,3-b]pyridin-2-yl)acetonitrile

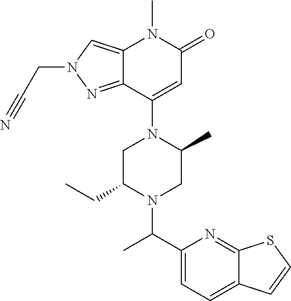 [SMILES: CC[C@@H]1CN(c2cc(=O)n(C)c3cn(CC#N)nc23)[C@@H](C)CN1C(C)c1ccc2ccsc2n1]

A solution of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (82 mg, 0.26 mmol), 1-(thieno[2,3-b]pyridin-6-yl)ethan-1-ol (50 mg, 0.26 mmol), (cyanomethyl)trimethylphosphonium iodide (190 mg, 0.78 mmol) and DIPEA (334 mg, 2,59 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography and then Prep-HPLC (Method A) to give the titled compound (42 mg). ¹H NMR (400 MHz, DMSO-d₆) δ 8.25 (dd, J=8.3, 3.2 Hz, 1H), 8.22 (s, 0.1H), 7.97 (d, J=4.5 Hz, 1H), 7.80 (dd, J=5.9, 4.3 Hz, 1H), 7.58 (dd, J=23.4, 8.3 Hz, 1H), 7.41 (dd, J=5.8, 4.9 Hz, 1H), 5.60 (d, J=7.8 Hz, 2H), 5.40 (s, 1H), 3.98 (q, J=6.7 Hz, 0.5H), 3.81 (q, J=6.7 Hz, 0.5H), 3.42 (d, J=13.1 Hz, 0.5H), 3.33 (s, 1H), 3.31-3.28 (m, 1H), 3.26 (d, J=1.1 Hz, 3H), 3.19 (d, J=10.3 Hz, 0.5H), 3.06 (d, J=10.6 Hz, 0.5H), 2.91 (d, J=3.6 Hz, 0.5H), 2.77-2.64 (m, 1H), 2.32 (d, J=10.8 Hz, 0.5H), 2.08 (d, J=12.0 Hz, 0.5H), 1.68-1.40 (m, 2H), 1.33 (dd, J=14.4, 6.6 Hz, 3H), 1,23 (d, J=6.4 Hz, 1.5H), 1.10 (d, J=6.4 Hz, 1.5H), 0.97 (t, J=7.3 Hz, 1.5H), 0.64 (t, J=7.3 Hz, 1.5H) ppm. MS: M/e 476 (M+1)⁺.

### Compound A131: 2-(7-((2S,5R)-5-ethyl-2-methyl-4-(1-(2-methylthieno[2,3-b]pyridin-6-yl)ethyl)piperazin-1-yl)-4-methyl-5-oxo-4,5-dihydro-2H-pyrazolo[4,3-b]pyridin-2-yl)acetonitrile

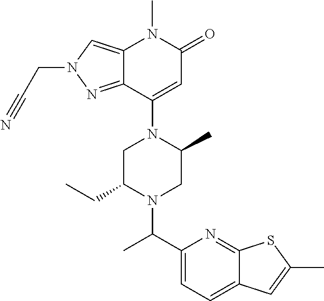 [SMILES: CC[C@@H]1CN(c2cc(=O)n(C)c3cn(CC#N)nc23)[C@@H](C)CN1C(C)c1ccc2cc(C)sc2n1]

A solution of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (82 mg, 0.26 mmol), 1-(2-methylthieno[2,3-b]pyridin-6-yl)ethan-1-ol (50 mg, 0.26 mmol), (cyanomethyl)trimethylphosphonium iodide (190 mg, 0.78 mmol) and DIPEA (334 mg, 2,59 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography and then Prep-HPLC (Method A) to give the titled compound (41 mg). ¹H NMR (400 MHz, DMSO-de) δ 8.15 (s, 0.1H), 8.07 (dd, J=8.3, 3.3 Hz, 1H), 7.97 (d, J=4.5 Hz, 1H), 7.50 (dd, .=24.6, 8.3 Hz, 1H), 7.08 (d, J=4.5 Hz, 1H), 5.59 (d, J=7.8 Hz, 2H), 5.39 (s, 1H), 3.93 (q, J=6.7 Hz, 0.5H), 3.77 (q, J=6.7 Hz, 0.5H), 3.41 (d, J=13.9 Hz, 0.5H), 3.33 (s, 1H), 3.30 (s, 1H), 3.26 (s, 3H), 3.17 (d, J=9.7 Hz, 0.5H), 3.04 (d, J=10.3 Hz, 0.5H), 2.90 (d, J=8.1 Hz, 0.5H), 2.77-2.64 (m, 1H), 2,56 (s, 3H), 2.31 (s, 0.5H), 2.08 (d, J=11.9 Hz, 0.5H), 1.65-1.38 (m, 2H), 1.32 (dd, J=15.2, 6.6 Hz, 3H), 1,22 (d, J=6.3 Hz, 1.5H), 1.09 (d, J=6.4 Hz, 1.5H), 0.96 (t, J=7.3 Hz, 1.5H), 0.64 (t, J=7.3 Hz, 1.5H) ppm. MS: M/e 490 (M+1)⁺.

### Compound A132: 2-(7-((2S,5R)-5-ethyl-4-(1-(2-fluorothieno[2,3-b]pyridin-6-yl)ethyl)-2-methylpiperazin-1-yl)-4-methyl-5-oxo-4,5-dihydro-2H-pyrazolo[4,3-b]pyridin-2-yl)acetonitrile

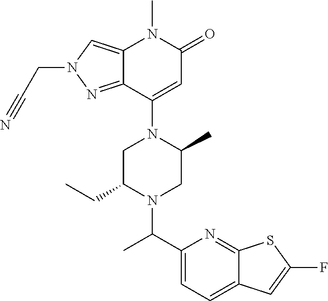 [SMILES: CC[C@@H]1CN(c2cc(=O)n(C)c3cn(CC#N)nc23)[C@@H](C)CN1C(C)c1ccc2cc(F)sc2n1]

A solution of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (80 mg, 0.25 mmol), 1-(2-fluorothieno[2,3-b]pyridin-6-yl)ethan-1-ol (50 mg, 0.25 mmol), (cyanomethyl)trimethylphosphonium iodide (185 mg, 0.76 mmol) and DIPEA (327 mg, 2,53 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography and then Prep-HPLC (Method A) to give the titled compound (37 mg). ¹H NMR (400 MHz, DMSO-d₆) δ 8.28 (s, 0.1H), 8.11 (dd, J=8.3, 3.0 Hz, 1H), 7.96 (d, J=4.1 Hz, 1H), 7.59 (dd, J=21.3, 8.3 Hz, 1H), 7.07 (t, J=4.1 Hz, 1H), 5.60 (d, J=7.2 Hz, 2H), 5.38 (s, 1H), 5.01-4.06 (m, 2H), 3.93 (q, J=6.5 Hz, 0.5H), 3.76 (q, J=6.4 Hz, 0.5H), 3.40 (d, J=11.4 Hz, 0.5H), 3.25 (s, 3H), 3.17 (d, J=10.1 Hz, 0.5H), 3.02 (d, J=10.5 Hz, 0.5H), 2.90 (dd, J=11.7, 3.9 Hz, 0.5H), 2.74-2.63 (m, 1H), 2.32 (s, 0.5H), 2.07 (d, J=10.2 Hz, 0.5H), 1.65-1.36 (m, 2H), 1.30 (dd, J=14.5, 6.6 Hz, 3H), 1,21 (d, J=6.4 Hz, 1.5H), 1.08 (d, J=6.4 Hz, 1.5H), 0.95 (t, J=7.3 Hz, 1.5H), 0.65 (t, .=7.3 Hz, 1.5H) ppm. MS: M/e 494 (M+1)⁺.

### Compound A133: 2-(7-((2S,5R)-2,5-diethyl-4-(1-(2-methylthiazolo[5,4-b]pyridin-5-yl)ethyl)piperazin-1-yl)-4-methyl-5-oxo-4,5-dihydro-2H-pyrazolo[4,3-b]pyridin-2-yl)acetonitrile

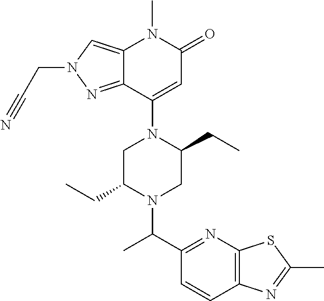 [SMILES: CC[C@H]1CN(C(C)c2ccc3nc(C)sc3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.10 mmol), 1-(2-methylthiazolo[5,4-b]pyridin-5-yl)ethan-1-ol (29 mg, 0.15 mmol) and (cyanomethyl)trimethylphosphonium iodide (73 mg, 0.30 mmol) in CH₃CN (0.5 mL) was added DIPEA (65 mg, 0.50 mmol). The mixture was stirred at 100° C. in a sealed tube for 16 hours. The mixture was cooled and diluted with EtOAc (10 mL), washed with brine (50 mL×3), dried overNa₂SO₄, filtered and concentrated to dryness. The resulting residue was purified by flash column chromatography and further separated into Compound A133a (12 mg) and Compound A133b (12 mg) by Prep-HPLC (Method A).

Compound A133a (the earlier peak): ¹H NMR (400 MHz, CD₃OD) δ 8.23 (d, J=8.8 Hz, 1H), 7.93 (s, 1H), 7.77 (d, J=8.4 Hz, 1H), 5.55 (s, 1H), 5.47 (s, 2H), 4.91-3.96 (m, 2H), 3.89 (q, J=6.0 Hz, 1H), 3.55 (d, J=12,4 Hz, 1H), 3.43 (s, 3H), 3.18 (d, J=10.0 Hz, 1H), 2.86 (s, 3H), 2.78 (dd, J=12,4, 3.6 Hz, 1H), 2.28 (d, J=12.0 Hz, 1H), 2.10-1.85 (m, 1H), 1.81-1.62 (m, 2H), 1.62-1.49 (m, 1H), 1.40 (d, J=6.8 Hz, 3H), 1.04 (t, J=7.2 Hz, 3H), 0.62 (t, J=7.2 Hz, 3H). MS: M/e 505 (M+1)⁺.

Compound A133b (the later peak): ¹H NMR (400 MHz, CD₃OD) δ 8.24 (d, J=8.4 Hz, 1H), 7.93 (s, 1H), 7.72 (d, J=8.4 Hz, 1H), 5.56 (s, 1H), 5.46 (s, 2H), 4.89-4.13 (m, 2H), 4.07 (q, J=6.8 Hz, 1H), 3.43 (s, 3H), 3.39-3.32 (m, 1H), 3.08-2.91 (m, 2H), 2.86 (s, 3H), 2,48-2.32 (m, 1H), 2.18-2.01 (m, 1H), 1.92-1.75 (m, 1H), 1.70-1.51 (m, 2H), 1.44 (d, J=6.4 Hz, 3H), 0.95 (t, J=7.2 Hz, 3H), 0.71 (t, J=7.2 Hz, 3H). MS: M/e 505 (M+1)⁺.

### Compound A134: 2-(7-((2S,5R)-2,5-diethyl-4-(1-(2-methylthiazolo[5,4-b]pyridin-5-yl)ethyl)piperazin-1-yl)-4-(methyl-d3)-5-oxo-4,5-dihydro-2H-pyrazolo[4,3-b]pyridin-2-yl)acetonitrile

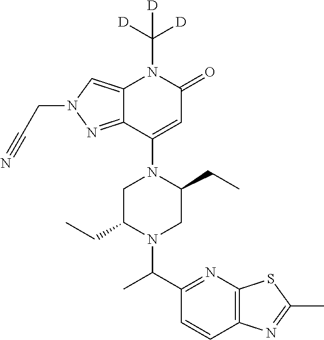 [SMILES: [2H]C([2H])([2H])n1c(=O)cc(N2C[C@@H](CC)N(C(C)c3ccc4nc(C)sc4n3)C[C@@H]2CC)c2nn(CC#N)cc21]

### Step A: 7-((2S,5R)-2,5-diethyl-4-(1-(2-methylthiazolo[5.4-b]pyridin-5-yl)ethyl)piperazin-1-yl)-4-(methyl-d3)-2-(tetrahydro-2H-pyran-2-yl)-2,4-dihydro-5H-pyrazolo[4,3-b]vridin-5-one

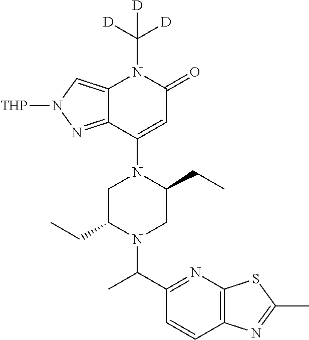

A mixture of 4-(methyl-d3)-5-oxo-2-(tetrahydro-2H-pyran-2-yl)-4,5-dihydro-2H-pyrazolo[4,3-b]pyridin-7-yl trifluoromethanesulfonate (50 mg, 0.13 mmol), 5-(1-((2R,5S)-2,5-diethylpiperazin-1-yl)ethyl)-2-methylthiazolo[5,4-b]pyridine (55 mg, 0.17 mmol) and DIPEA (100 mg, 0.77 mmol) in MeCN (1.0 mL) was stirred at 100° C. in a sealed tube for 16 hours. The mixture was diluted with EA (10 mL), washed with brine (5 mL×3), dried over Na₂SO₄, filtered and concentrated to dryness. The resulted residue was purified by Prep-TLC to give the titled compound (35 mg, 49%). MS: M/e 553 (M+1)⁺.

### Step B: 7-((2S,5R)-2,5-diethyl-4-(1-(2-methylthiazolo[5.4-b]pyridin-5-yl)ethyl)piperazin-1-yl)-4-(methyl-d3)-2,4-dihydro-5H-pyrazolo[4,3-b]pyridin-5-one

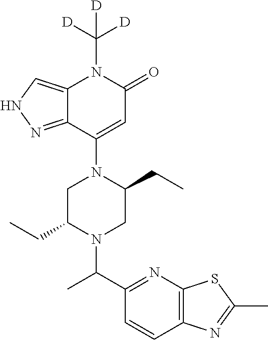 [SMILES: [2H]C([2H])([2H])n1c(=O)cc(N2C[C@@H](CC)N(C(C)c3ccc4nc(C)sc4n3)C[C@@H]2CC)c2n[nH]cc21]

To a solution of 7-((2S,5R)-2,5-diethyl-4-(1-(2-methylthiazolo[5,4-b]pyridin-5-yl)ethyl)piperazin-1-yl)-4-(methyl-d3)-2-(tetrahydro-2H-pyran-2-yl)-2,4-dihydro-5H-pyrazolo[4,3-b]pyridin-5-one (35 mg, 0.063 mmol) in MeOH (2 mL) was added HCl/1,4-dioxane (4 M, 2 mL) at rt and the mixture was stirred for 3 hours. The mixture was concentrated to dryness to give the titled compound (28 mg, crude). MS: M/e 469 (M+1)⁺.

### Step C: 2-(7-((2S,5R)-2,5-diethyl-4-(1-(2-methylthiazolo[5.4-b]pyridin-5-yl)ethyl)piperazin-1-yl)-4-(methyl-d3)-5-oxo-4,5-dihydro-2H-pyrazolo[4,3-blpyridin-2-yl)acetonitrile

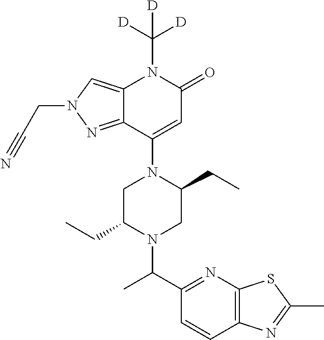 [SMILES: [2H]C([2H])([2H])n1c(=O)cc(N2C[C@@H](CC)N(C(C)c3ccc4nc(C)sc4n3)C[C@@H]2CC)c2nn(CC#N)cc21]

A mixture of 7-((2S,5R)-2,5-diethyl-4-(1-(2-methylthiazolo[5,4-b]pyridin-5-yl)ethyl)piperazin-1-yl)-4-(methyl-d3)-2,4-dihydro-5H-pyrazolo[4,3-b]pyridin-5-one (28 mg, 0.06 mmol) and K₂CO₃ (100 mg, 0.72 mmol) in DMF (1 mL) was added 2-chloroacetonitrile (30 mg, 0.40 mmol) at room temperature and the mixture was stirred for 16 hours. The mixture was diluted with EtOAc (10 mL) washed with brine (3 mL×3), dried over Na₂SO₄, and concentrated to dryness. The resulted residue was purified by Prep-TLC and Prep-HPLC (Method A) to give titled Compound A134a (4 mg) and Compound A134b (4 mg).

Compound A134a (the earlier peak): ¹H NMR (400 MHz, CD₃OD) δ 8.22 (d, J=8.4 Hz, 1H), 7.93 (s, 1H), 7.76 (d, J=8.4 Hz, 1H), 5.55 (s, 1H), 5.47 (s, 2H), 4.83-3.75 (m, 3H), 3.56 (d, J=13.2 Hz, 1H), 3.20 (d, J=10.4 Hz, 1H), 2.93-2.75 (m, 4H), 2.31 (d, J=11.8 Hz, 1H), 2.08-1.89 (m, 1H), 1.79-1.63 (m, 2H), 1.62-1.50 (m, 1H), 1.41 (d, J=6.8 Hz, 3H), 1.04 (t, J=7.2 Hz, 3H), 0.63 (t, J=7.2 Hz, 3H). MS: M/e 508 (M+1)⁺.

Compound A134b (the later peak): ¹H NMR (400 MHz, CD₃OD) δ 8.24 (d, J=8.4 Hz, 1H), 7.92 (s, 1H), 7.71 (d, J=8.4 Hz, 1H), 5.55 (s, 1H), 5.45 (s, 2H), 4.86-3.95 (m, 3H), 3.35 (d, J=13.6 Hz, 1H), 3.13-2.93 (m, 2H), 2.86 (s, 3H), 2,52-2.34 (m, 1H), 2.17-1.98 (m, 1H), 1.91-1.75 (m, 1H), 1.68-1.52 (m, 2H), 1.45 (d, J=6.4 Hz, 3H), 0.95 (t, 0.1=7.2 Hz, 3H), 0.71 (t, J=7.2 Hz, 3H). MS: M/e 508 (M+1)”.

### Compound A135: 2-(7-((2S,5R)-2,5-diethyl-4-(1-(4-(trifluoromethyl) phenyl)propyl)piperazin-1-yl)-4-methyl-5-oxo-4,5-dihydro-2H-pyrazolo[4,3-b]pyridin-2-yl)acetonitrile

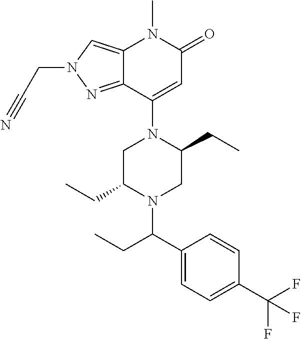 [SMILES: CCC(c1ccc(C(F)(F)F)cc1)N1C[C@H](CC)N(c2cc(=O)n(C)c3cn(CC#N)nc23)C[C@H]1CC]

A mixture of 1-(4-(trifluoromethyl)phenyl)propan-1-ol (122 mg, 0.6 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (100 mg, 0.3 mmol), (cyanomethyl) trimethylphosphonium iodide (290 mg, 1,2 mmol) and DIPEA (155 mg, 1,2 mmol) in MeCN (3 mL) was stirred at 100° C. overnight in a sealed tube. The reaction mixture was diluted with EtOAc (20 mL), washed with water (10 mL×2), dried over Na₂SO₄ and concentrated to dryness. The resulting residue was purified by Prep-HPLC (Method B) to give the titled Compound A135a (27 mg) and Compound A135b (24 mg).

Compound A135a (the earlier peak): ¹H NMR (400 MHz, CDCl₃-d) 6 7.59 (d, .=7.6 Hz, 2H), 7.42 (d, J=7.7 Hz, 2H), 7.37 (s, 1H), 5.58 (s, 1H), 5.17-5.07 (m, 2H), 4.40 (s, 1H), 3.55 (d, J=5.5 Hz, 1H), 3.41 (s, 3H), 3.19 (d, J=12.8 Hz, 1H), 2.96 (d, J=11.7 Hz, 1H), 2.84 (d, J=11.4 Hz, 1H), 2.33 (d, J=9.0 Hz, 1H), 2.06 (d, J=5.8 Hz, 1H), 1.93 (s, 1H), 1.75 (s, 1H), 1.57 (s, 3H), 1.43 (s, 1H), 0.93 (s, 3H), 0.76-0.44 (m, 6H) ppm. MS: M/e 515 (M+1)⁺.

Compound A135b (the later peak): ¹H NMR (400 MHz, CDCl₃-d) δ 7.57 (d, J=7.6 Hz, 2H), 7.45 (d, J=7.7 Hz, 2H), 7.37 (s, 1H), 5.59 (s, 1H), 5.19-5.06 (m, 2H), 3.41 (s, 6H), 3.09 (d, J=9.7 Hz, 1H), 2.62 (d, J=11.2 Hz, 1H), 2.23 (d, J=12.1 Hz, 1H), 1.87 (s, 2H), 1.70 (s, 1H), 1.57 (s, 3H), 1.48 (d, J=7.8 Hz, 1H), 0.97 (s, 3H), 0.64 (t, J=6.9 Hz, 3H), 0.55 (s, 3H) ppm. MS: M/e 515 (M+1)⁺.

### Compound A136: 7-((2S,5R)-2,5-diethyl-4-(1-(2-methylthiazolo[5.4-b1 pyridin-5-yl)ethyl) piperazin-1-yl)-4-methyl-2-(oxetan-3-yl)-2,4-dihydro-5H-pyrazolo4,3-b]pyridin-5-one

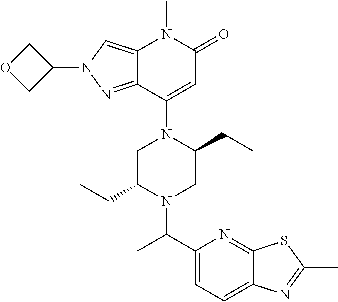 [SMILES: CC[C@H]1CN(C(C)c2ccc3nc(C)sc3n2)[C@H](CC)CN1c1cc(=O)n(C)c2cn(C3COC3)nc12]

To a solution of 7-((2S,5R)-2,5-diethyl-4-(1-(2-methylthiazolo[5,4-b]pyridin-5-yl)ethyl)piperazin-1-yl)-4-methyl-2,4-dihydro-5H-pyrazolo[4,3-b]pyridin-5-one (50 mg, 0.11 mmol) and K₂CO₃ (45 mg, 0.32 mmol) in DMF (10 mL) was added 3-iodooxetane (20 mg, 0.11 mmol). The reaction mixture was stirred at room temperature overnight. The reaction mixture was diluted with water and extracted with EA (80 mL×2). The combined organic layers were washed with brine, dried over Na₂SO₄, filtered and concentrated to dryness. The resulting residue was purified Prep-HPLC (Method A) to give the titeld Compound A136a (11 mg) and Compound A136b (9 mg).

Compound A136a (the earlier peak): ¹H NMR (400 MHz, CD₃OD) δ 8.23 (d, J=8.4 Hz, 1H), 7.95 (s, 1H), 7.77 (d, J=8.3 Hz, 1H), 5.74-5.61 (m, 1H), 5.55 (s, 1H), 5.13-4.99 (m, 4H), 3.94 (s, 1H), 3.58 (d, J=13.5 Hz, 1H), 3.44 (s, 3H), 3.29-3.12 (m, 3H), 2.86 (s, 3H), 2.85-2.75 (m, 1H), 2,42-2.26 (m, 1H), 2.06-1.96 (m, 1H), 1.80-1.65 (m, 2H), 1.62-1.52 (m, 1H), 1.43 (d, J=6.1 Hz, 3H), 1.07 (t, J=7.1 Hz, 3H), 0.64 (t, J=7.2 Hz, 3H). MS: M/e 522 (M+1)⁺.

Compound A136b (the later peak): ¹H NMR (400 MHz, CD₃OD) δ 8.24 (d, J=8.5 Hz, 1H), 7.94 (s, 1H), 7.72 (d, J=8.3 Hz, 1H), 5.70-5.61 (m, 1H), 5.55 (s, 1H), 5.10-5.00 (m, 4H), 4.14-4.05 (m, 1H), 3.43 (s, 3H), 3.38 (d, J=15.2 Hz, 3H), 3.08 (d, J=12.8 Hz, 1H), 2.99 (d, J=11.1 Hz, 1H), 2.86 (s, 3H), 2,44 (s, 1H), 2.20-2.09 (m, 1H), 1.88-1.78 (m, 1H), 1.67-1.57 (m, 2H), 1.46 (d, J=6.4 Hz, 3H), 0.98 (t, J=7.4 Hz, 3H), 0.73 (t, J=7.2 Hz, 3H). MS: M/e 522 (M+1)⁺.

### Compound A137: 2-(but-2-yn-1-yl-7-((2S,5R)-2,5-diethyl-4-(1-(2-methylthiazolo[5.4-b]pyridin-5-yl)ethyl)piperazin-1-yl-4-methyl-2,4-dihydro-5H-pyrazolo[4,3-b]pyridin-5-one

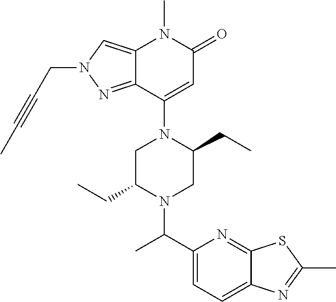 [SMILES: CC#CCn1cc2c(n1)c(N1C[C@@H](CC)N(C(C)c3ccc4nc(C)sc4n3)C[C@@H]1CC)cc(=O)n2C]

### Step A: 7-((2S,5R)-2,5-diethyl-4-(1-(2-methylthiazolo[5.4-b]pyridin-5-yl)ethyl) piperazin-1-yl) 4-methyl-2-(tetrahydro-2H-pyran-2-yl)-2,4-dihydro-5H-pyrazolo[4,3-b]pyridin-5-one

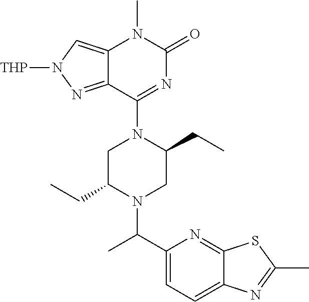

To a solution of 4-methyl-5-oxo-2-(tetrahydro-2H-pyran-2-yl)-4,5-dihydro-2H-pyrazolo[4,3-b]pyridin-7-yl trifluoromethanesulfonate (200 mg, 0.52 mmol) in CH₃CN (3 mL) and was added 5-(1-((2R,5S)-2,5-diethylpiperazin-1-yl) ethyl)-2-methylthiazolo[5,4-b]pyridine (167 mg, 0.52 mmol) and DIPEA (339 mg, 2.62 mmol). The resulting mixture was stirred at 105° C. overnight. The reaction mixture was diluted with water and extracted with EA (80 mL×2). The combined organic layers were washed with brine, dried over Na₂SO₄, filtered and concentrated to dryness. The resulting residue was purified by flash column chromatography to give the titled Compound (200 mg, 69%). MS: M/e 550 (M+1)⁺.

### Step B: 7-((2S,5R˜2,5-diethyl-4-(1-(2-methylthiazolo[5.4-b]pyridin-5-yl) ethyl)piperazin-1-yl)-4-methyl-2,4-dihydro-5H-pyrazolo[4,3-b]pyridin-5-one

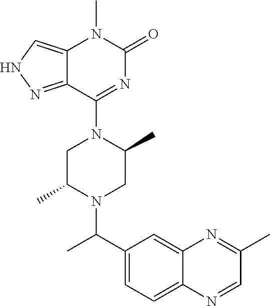 [SMILES: Cc1cnc2ccc(C(C)N3C[C@H](C)N(c4nc(=O)n(C)c5c[nH]nc45)C[C@H]3C)cc2n1]

To a stirred solution of 7-((2S,5R)-2,5-diethyl-4-(1-(2-methylthiazolo[5,4-b]pyridin-5-yl)ethyl)piperazin-1-yl)-4-methyl-2-(tetrahydro-2H-pyran-2-yl)-2,4-dihydro-5H-pyrazolo[4,3-b]pyridin-5-one (200 mg, 0.36 mmol) in MeOH(2 mL) was added HCl dioxane solution (2 mL, 8 mmol, 4M). The resulting mixture was stirred at room temperature for 2 hours. The reaction mixture was concentrated to dryness. The resulting residue (160 mg, crude) was used in the next step directly without further purification. MS: M/e 466 (M+1)⁺.

### Step C: 2-(but-2-vn-1-yl)-7-((2S,5R)-2,5-diethyl-4-(1-(2-methylthiazolo[5.4-b]pyridin-5-yl)ethyl)piperazin-1-yl)-4-methyl-2,4-dihydro-5H-pyrazolo[4,3-b]pyridin-5-one

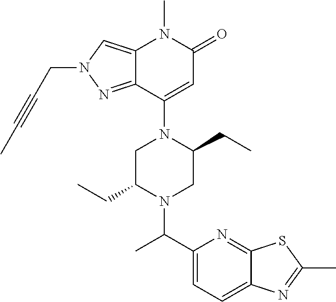 [SMILES: CC#CCn1cc2c(n1)c(N1C[C@@H](CC)N(C(C)c3ccc4nc(C)sc4n3)C[C@@H]1CC)cc(=O)n2C]

To a solution of 7-((2S,5R)-2,5-diethyl-4-(1-(2-methylthiazolo[5,4-b]pyridin-5-yl) ethyl)piperazin-1-yl)-4-methyl-2,4-dihydro-5H-pyrazolo[4,3-b]pyridin-5-one (50 mg, 0.11 mmol) and K₂CO₃ (45 mg, 0.32 mmol) in DMF (10 mL) was added 1-bromobut-2-yne (14 mg, 0.11 mmol). The reaction mixture was stirred at room temperature overnight. The reaction mixture was diluted with water and extracted with EA (80 mL×2). The combined organic layers were washed with brine, dried over Na₂SO₄, filtered and concentrated to dryness. The resulting residue was purified Prep-HPLC (Method A) to give the titled compound (20 mg, 36%). ¹H NMR (400 MHz, CD₃OD) δ 8.24 (d, J=9.1 Hz, 1H), 7.90 (d, J=4.3 Hz, 1H), 7.78-7.70 (m, 1H), 5.52 (s, 1H), 5.03 (s, 2H), 4.10 (s, 1H), 3.59-3.47 (m, 1H), 3.44 (s, 3H), 3.27-3.17 (m, 1H), 3.14-2.92 (m, 2H), 2.86 (s, 3H), 2,51-2.32 (m, 1H), 2.27-1.88 (m, 2H), 1.84 (s, 3H), 1.77-1.67 (m, 1H), 1.66-1.50 (m, 2H), 1.45 (d, J=6.5 Hz, 3H), 1.07-0.93 (m, 3H), 0.74-0.61 (m, 3H). MS: M/e 518 (M+1)⁺.

### Compound A138: 2-(but-2-yn-1-yl)-7-((2S,5R)-2,5-diethyl-4-(1-(7-fluoro-2-methylbenzo[d]thiazol-6-yl)ethyl)piperazin-1-yl)-4-methyl-2,4-dihydro-5H-pyrazolo[4,3-b]pyridin-5-one

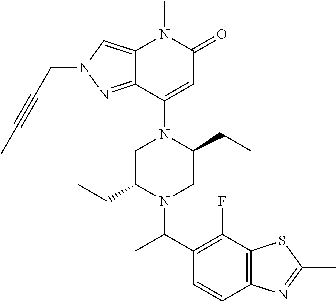 [SMILES: CC#CCn1cc2c(n1)c(N1C[C@@H](CC)N(C(C)c3ccc4nc(C)sc4c3F)C[C@@H]1CC)cc(=O)n2C]

To a solution of 2-(but-2-yn-1-yl)-7-((2S,5R)-2,5-diethylpiperazin-1-yl)-4-methyl-2,4-dihydro-5H-pyrazolo[4,3-b]pyridin-5-one (100 mg, 0.20 mmol) (100 mg, 0.29 mmol) in CH₃CN (5 mL) was added 1-(5-fluorobenzo[d]thiazol-6-yl)ethan-1-ol (122 mg, 0.58 mmol), (cyanomethyl)trimethylphosphonium iodide (213 mg, 0.88 mmol) and DIPEA(378 mg, 2.9 mmol).The resulting mixture was stirred at 105° C. overnight. The reaction solvent was removed under reduced pressure. The resulting residue was purified by flash column chromatography to give the titled compound(crude), which was further purified to give the titled Compound A138a (16 mg) and Compound A138b (17 mg) by Prep-HPLC (Method A).

Compound A138a (the earlier peak): ¹H NMR (400 MHz, CD₃OD) δ 7.88 (s, 1H), 7.75 (d, J=8.5 Hz, 1H), 7.72-7.63 (m, 1H), 5.51 (s, 1H), 5.02 (d, J=2.3 Hz, 2H), 4.86-4.41 (m, 1H), 4.30 (q, J=6.5 Hz, 1H), 3.43 (s, 3H), 3.25 (s, 2H), 3.04-2.90 (m, 2H), 2.85 (s, 3H), 2.37 (s, 1H), 2.16-1.99 (m, 1H), 1.92-1.73 (m, 4H), 1.63-1.48 (m, 2H), 1.42 (d, J=6.5 Hz, 3H), 0.96 (t, J=7.4 Hz, 3H), 0.70 (t, J=7.3 Hz, 3H). ppm. MS: M/e 535 (M+1)⁺.

Compound A138b (the later peak): ¹H NMR (400 MHz, CD₃OD) δ 7.89 (s, 1H), 7.81-7.66 (m, 2H), 5.51 (s, 1H), 5.03 (d, J=2.3 Hz, 2H), 4.86-4.34 (m, 2H), 4.16 (q, J=6.4 Hz, 1H), 3.49 (d, J=12.8 Hz, 1H), 3.43 (s, 3H), 3.16 (d, J=10.1 Hz, 1H), 2.84 (s, 3H), 2.70 (d, J=9.0 Hz, 1H), 2.33 (d, J=12.0 Hz, 1H), 1.96 (s, 1H), 1.84 (t, J=2.3 Hz, 3H), 1.76-1.49 (m, 3H), 1.38 (d, J=6.6 Hz, 3H), 1.03 (t, J=7.3 Hz, 3H), 0.59 (t, J=7.2 Hz, 3H). ppm. MS: M/e 535 (M+1)⁺.

### Compound A139: 2-(but-2-yn-1-yl)-7-((2S,5R)-2,5-diethyl-4-(1-(4-fluorobenzo[d]thiazol-5-yl)ethyl)piperazin-1-yl)-4-methyl-2,4-dihydro-5H-pyrazolo[4,3-b]pyridin-5-one

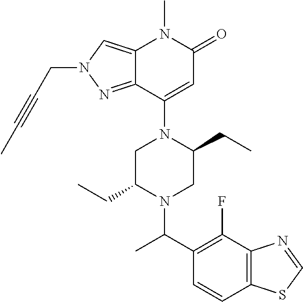 [SMILES: CC#CCn1cc2c(n1)c(N1C[C@@H](CC)N(C(C)c3ccc4scnc4c3F)C[C@@H]1CC)cc(=O)n2C]

To a solution of 2-(but-2-yn-1-yl)-7-((2S,5R)-2,5-diethylpiperazin-1-yl)-4-methyl-2,4-dihydro-5H-pyrazolo[4,3-b]pyridin-5-one (30 mg, 0.087 mmol), 1-(4-fluorobenzo[d]thiazol-5-yl)ethan-1-ol (40 mg, 0.2 mmol) and (cyanomethyl)trimethylphosphonium iodide (120 mg, 0.5 mmol) in CH₃CN (3 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified and separated into Compound A139a (1.9 mg) and Compound A139b (1.7 mg) by Prep-HPLC (Method A).

Compound A139a (the earlier peak): ¹H NMR (400 MHz, CD₃OD) δ 9.26 (s, 1H), 7.89 (d, J=8.1 Hz, 2H), 7.71 (d, J=5.9 Hz, 1H), 5.52 (s, 1H), 5.01 (s, 2H), 4.40 (d, J=6.6 Hz, 1H), 3.43 (s, 3H), 3.26 (s, 2H), 3.12 (s, 1H), 3.02 (d, J=11.1 Hz, 1H), 2.95 (m, 1H), 2.38 (m, 1H), 2.08 (m, 1H), 1.83 (m, 4H), 1.57 (m, 2H), 1.44 (d, J=6.5 Hz, 3H), 0.96 (t, J=7.4 Hz, 3H), 0.71 (t, 0.1=7.3 Hz, 3H) ppm. MS: M/e 521 (M+1)⁺.

Compound A139b (the later peak): ¹H NMR (400 MHz, CD₃OD) δ 9.25 (s, 1H), 7.87 (m, 2H), 7.79 (m, 1H), 5.51 (s, 1H), 5.03 (s, 2H), 4.25 (d, J=6.5 Hz, 1H), 3.49 (d, 0.1=15.5 Hz, 2H), 3.43 (s, 3H), 3.16 (m, 2H), 2.74 (d, J=9.7 Hz, 1H), 2.35 (d, J=12.3 Hz, 1H), 1.95 (s, 1H), 1.84 (s, 3H), 1.65 (m, 3H), 1.40 (d, J=6.5 Hz, 3H), 1.04 (t, J=7.1 Hz, 3H), 0.59 (t, J=7.0 Hz, 3H) ppm. MS: M/e 521 (M+1)⁺.

### Compound A140: 2-(cyanomethyl)-7-((2S,5R)-2,5-diethyl-4-(1-(2-methylthiazolo[5,4-b]pyridin-5-yl)ethyl)piperazin-1-yl)-4-methyl-5-oxo-4,5-dihydro-2H-pyrazolo[4,3-b]pyridine-6-carbonitrile

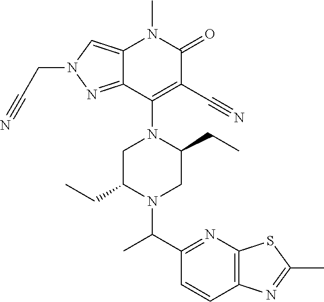 [SMILES: CC[C@H]1CN(C(C)c2ccc3nc(C)sc3n2)[C@H](CC)CN1c1c(C#N)c(=O)n(C)c2cn(CC#N)nc12]

### Step A: 2-(6-bromo-7-((2S,5R)-2,5-diethyl-4-(1-(2-methylthiazolo[5.4-b]pyridin -5-yl)ethyl)piperazin-1-yl)-4-methyl-5-oxo-4,5-dihydro-2H-pyrazolo[4,3-b]pyridin-2-yl)acetonitrile

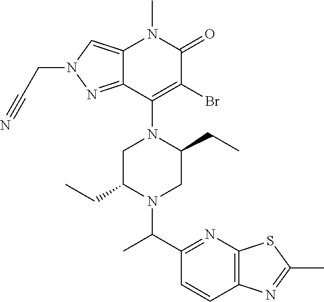 [SMILES: CC[C@H]1CN(C(C)c2ccc3nc(C)sc3n2)[C@H](CC)CN1c1c(Br)c(=O)n(C)c2cn(CC#N)nc12]

To a solution of 2-(7-((2S,5R)-2,5-diethyl-4-(1-(2-methylthiazolo[5,4-b]pyridin-5-yl)ethyl)piperazin-1-yl)-4-methyl-5-oxo-4,5-dihydro-2H-pyrazolo[4,3-b]pyridin-2-yl)acetonitrile (310 mg, 0.61 mmol) in CH₃CN (5 mL) and was added NBS (131 mg, 0.73 mmol).The resulting mixture was stirred for 10 mins at room temperature. The reaction solvent was removed under reduced pressure. The resulting residue was purified by flash column chromatography to give the titled compound (180 mg, 50%). MS: M/e 583 (M+1)⁺.

### Step B: 2-(cyanomethyl)-7-((2S,5R)-2,5-diethyl-4-(1-(2-methylthiazolo[5.4-b]pyridin-5-yl)ethyl)piperazin-1-yl)-4-methyl-5-oxo-4,5-dihydro-2H-pyrazolo[4,3-b]pyridine-6-carbonitrile

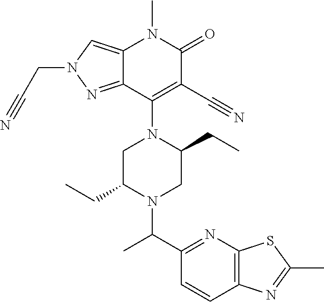 [SMILES: CC[C@H]1CN(C(C)c2ccc3nc(C)sc3n2)[C@H](CC)CN1c1c(C#N)c(=O)n(C)c2cn(CC#N)nc12]

To a solution of 2-(6-bromo-7-((2S,5R)-2,5-diethyl-4-(1-(2-methylthiazolo[5,4-b]pyridin-5-yl)ethyl) piperazin-1-yl)-4-methyl-5-oxo-4,5-dihydro-2H-pyrazolo[4,3-b]pyridin-2-yl)acetonitrile (180 mg, 0.3 mmol), Zn(CN)₂ (360 mg, 3 mmol) and Pd(PPh₃)₄ (173 mg, 0.15 mmol) in DMF (3 mL).The resulting mixture was stirred at 100° C. overnight. The reaction mixture was diluted with DCM and washed with water, dried over Na₂SO₄ and concentrated to dryness. The resulting residue was purified by flash column chromatography to give the titled compound(crude), which was further separated into Compound A140a (27 mg) and Compound A140b (21 mg) by Prep-HPLC (Method A).

Compound A140a (the earlier peak): ¹H NMR (400 MHz, CD₃OD) δ 8.23 (d, J=8.5 Hz, 1H), 8.01 (s, 1H), 7.77 (d, .=8.5 Hz, 1H), 5.51 (s, 2H), 5.40-5.03 (m, 1H), 4.71-4.09 (m, 1H), 4.08-3.93 (m, 1H), 3.89 (q, J=6.2 Hz, 1H), 3.43 (s, 3H), 3.30-3.16 (m, 2H), 3.14-2.95 (m, 1H), 2.85 (s, 3H), 2.39-2.11 (m, 2H), 1.65-1.49 (m, 2H), 1.40 (d, J=6.6 Hz, 3H), 0.87 (t, J=7.2 Hz, 3H), 0.78-0.61 (m, 3H) ppm. MS: M/e 530 (M+1)⁺.

Compound A140b (the later peak): ¹H NMR (400 MHz, CD₃OD) δ 8.25 (d, J=8.4 Hz, 1H), 8.01 (s, 1H), 7.74 (d, J=8.5 Hz, 1H), 5.53 (s, 2H), 5.48-4.97 (m, 1H), 4.81-4.52 (m, 1H), 4.06 (d, J=6.6 Hz, 1H), 3.87-3.72 (m, 1H), 3.43 (s, 3H), 3.24-2.96 (m, 2H), 2.85 (s, 3H), 2,44 (d, J=9.9 Hz, 1H), 2.39-1.53 (m, 4H), 1.43 (d, J=6.6 Hz, 3H), 0.89 (t, J=7.1 Hz, 3H), 0.56 (t, J=7.4 Hz, 3H) ppm. MS: M/e 530 (M+1)”.

### Compound A141: 2-(but-2-yn-1-yl)-7-((2S,5R)-2,5-diethyl-4-(1-(4-fluoro-2-methylbenzo[d]thiazol-5-yl)ethyl)piperazin-1-yl)-4-methyl-2,4-dihydro-5H-pyrazolo[4,3-b]pyridin-5-one

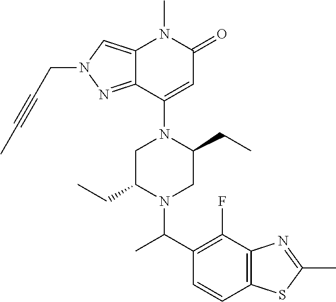 [SMILES: CC#CCn1cc2c(n1)c(N1C[C@@H](CC)N(C(C)c3ccc4sc(C)nc4c3F)C[C@@H]1CC)cc(=O)n2C]

A solution of 2-(but-2-yn-1-yl)-7-((2S,5R)-2,5-diethylpiperazin-1-yl)-4-methyl-2,4-dihydro-5H-pyrazolo[4,3-b]pyridin-5-one (34 mg, 0.1 mmol), 1-(4-fluoro-2-methylbenzo[d]thiazol-5-yl)ethan-1-ol (33 mg, 0.15 mmol), (cyanomethyl)trimethylphosphonium iodide (120 mg, 0.5 mmol) and DIPEA (129 mg, 1 mmol) in CH₃CN (3 ml) was stirred at 100° C. for overnight. The reaction was diluted with EtOAc (15 ml) and washed with brine (10 ml). The organic layer was concentrated under reduced pressure. The resulting residue was purified by flash column chromatography and separated into Compound A141a (2,5 mg, 0.93%) and Compound A141b (3.2 mg, 1,2%) by Prep-HPLC (Method A).

Compound A141a (the earlier peak): ¹H NMR (400 MHz, CD₃OD-d₆) δ 7.89 (s, 1H), 7.74 (d, J=8.4 Hz, 1H), 7.60 (dd, J=8.3, 6.0 Hz, 1H), 5.52 (s, 1H), 5.02 (d, J=2.1 Hz, 2H), 4.36 (q, J=6.5 Hz, 1H), 3.44 (s, 3H), 3.26-3.24 (m, 2H), 3.02-2.98 (m, 1H), 2.95-2.93 (m, 1H), 2.85 (s, 3H), 2,40-2.36 (m, 1H), 2.10-2.06 (m, 2H), 1.85-1.83 (m, 4H), 1.57-1.55 (m, 2H), 1.43 (d, J=6.5 Hz, 3H), 0.96 (t, J=7.3 Hz, 3H), 0.71 (t, J=7.3 Hz, 3H) ppm. MS: M/e 535 (M+1)⁺.

Compound A141b (the later peak): ¹H NMR (400 MHz, CD₃OD-d₆) δ 7.90 (s, 1H), 7.70 (m, 2H), 5.51 (s, 1H), 5.04 (d, J=2.2 Hz, 2H), 4.22 (d, J=6.5 Hz, 1H), 3.48-3.46 (m, 4H), 3.28-3.26 (m, 1H), 3.18-3.16 (m, 1H), 2.85 (s, 3H), 2.73-2.71 (m, 1H), 2.36-2.34 (m, 1H), 2.06-2.04 (m, 2H), 1.85 (s, 3H), 1.67-1.65 (m, 3H), 1.39 (d, J=6.5 Hz, 3H), 1.04 (t, J=7.3 Hz, 3H), 0.60 (t, J=7.0 Hz, 3H) ppm. MS: We 535 (M+1)⁺.

### Compound A142: 2-(7-((2S,5R)-2,5-diethyl-4-(1-(2-methylthiazolo[5,4-b]pyridin-5-yl)ethyl-2,2,2-d3)piperazin-1-yl)-4-(methyl-d3)-5-oxo-4,5-dihydro-2H-pyrazolo[4,3-b]pyridin-2-yl)acetonitrile

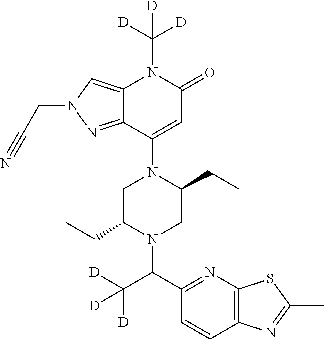 [SMILES: [2H]C([2H])([2H])C(c1ccc2nc(C)sc2n1)N1C[C@H](CC)N(c2cc(=O)n(C([2H])([2H])[2H])c3cn(CC#N)nc23)C[C@H]1CC]

### Step A: 1-42-methylthiazolo[5.4-b]pyridin-5-yl)ethan-2.2.2-d3-1-ol

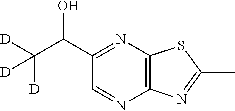 [SMILES: [2H]C([2H])([2H])C(O)c1cnc2nc(C)sc2n1]

To a solution of 2-methylthiazolo[5,4-b]pyridine-5-carbaldehyde (178 mg, 1 mmol) in THF (10 mL) was added a solution of CD3Mgl in THF (1,2 mL, 1 mol/L). The reaction mixture was stirred at room temperature for 2 hours. The mixture was diluted with saturated NH₄Cl (20 mL), extracted with EtOAc (60 mL×2), washed with brine, dried over Na₂SO₄, filtered, and concentrated to dryness. The residue was purified by flash column chromatography to give the titled compound (130 mg, 65%). MS: M/e 198 (M+1)⁺.

### Step B: tert-butyl (2S,5R)-2,5-diethyl-4-(1-(2-methylthiazolo[5.4-b]pyridin-5-yl)ethyl-2.2.2-d3)piperazine-1-carboxylate

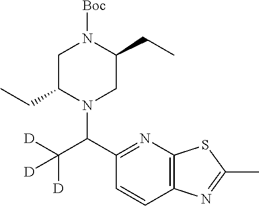 [SMILES: [2H]C([2H])([2H])C(c1ccc2nc(C)sc2n1)N1C[C@H](CC)N(C(=O)OC(C)(C)C)C[C@H]1CC]

To a solution of tert-butyl (2S,5R)-2,5-diethylpiperazine-1-carboxylate (160 mg, 0.66 mmol), 1-(2-methylthiazolo[5,4-b]pyridin-5-yl)ethan-2,2,2-d₃-1-ol (130 mg, 0.66 mmol) and DIPEA (426 mg, 3.3 mmol) in CH₃CN (2 ml ) was added (cyanomethyl)trimethylphosphonium iodide (320 mg, 1.32 mmol). The mixture was stirred at 103° C. in a sealed bottle for 16 hours. The reaction was cooled to room temperature, diluted by adding water, extracted with EtOAc (60 mL×2), washed with brine, dried over Na₂SO₄, filtered and concentrated to dryness. The residue was purified by flash column chromatography to give the titled compound (200 mg, 70%). MS: M/e 422 (M+1)⁺.

### Step C: 5-(1-((2R,5S)-2,5-diethylpiperazin-1-yl)ethyl-2.2.2-d)-2-methylthiazolo[5.4-b]pyridine

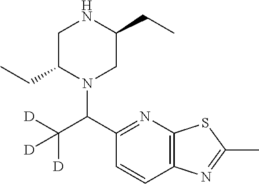 [SMILES: [2H]C([2H])([2H])C(c1ccc2nc(C)sc2n1)N1C[C@H](CC)NC[C@H]1CC]

To a solution of tert-butyl (2S,5R)-2,5-diethyl-4-(1-(2-methylthiazolo[5,4-b]pyridin-5-yl)ethyl-2,2,2-d₃)piperazine-1-carboxylate (200 mg, 0.475 mmol) in DCM (8 mL) was added TMSOTf (210 mg, 0.95 mmol). The resulted mixture was stirred at room temperature for 2 hours. The reaction mixture was quenched with aq. NaHCO₃, then extracted with CH₂Cl₂/IPA (3/1, 40 mL x 5). The combined organic layers were dried over Na₂SO₄, concentrated to give the titled compound (143 mg, 94%). MS: M/e 322 (M+1)⁺.

### Step D: 7-((2S,5R)-2,5-diethyl-4-(1-(2-methylthiazolo[5.4-b]pyridin-5-yl)ethyl-2.2.2-da)lpiperazin-1-yl)-4-(methyl-d₃)-2-(tetrahydro-2H-nyran-2-yl)-2,4-dihydro-5H-pyrazolo[4,3-b]pyridin-5-one

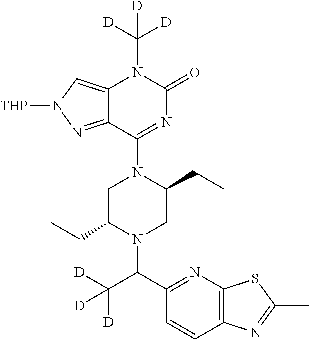

To a mixture of 4-(methyl-d3)-5-oxo-2-(tetrahydro-2H-pyran-2-yl)-4,5-dihydro-2H-pyrazolo[4,3-b]pyridin-7-yl trifluoromethanesulfonate (205 mg, 0.53 mmol) and 5-(1-((2R,5S)-2,5-diethylpiperazin-1-yl)ethyl-2,2,2-d3)-2-methylthiazolo[5,4-b]pyridine (143 mg, 0.445 mmol) in CH₃CN (5 mL) was added DIEA (115 mg, 0.89 mmol). The reaction was stirred at 100° C. for 16 hours. The reaction was cooled to room temperature, quenched with ice-water, extracted with EtOAc (80 mL×2), washed with brine, dried over Na₂SO₄, filtered, and concentrated. The residue was purified by flash column chromatography to give the titled compound (90 mg, 36%). MS: M/e 556 (M+1)⁺.

### Step E: 7-((2S,5R-2,5-diethyl-4-(1-(2-methylthiazolo[5.4-b]pyridin-5-yl)ethyl-2.2.2-d3)piperazin-1-yl)-4-(methyl-d3)-2,4-dihydro-5H-pyrazolo[4,3-b]pyridin-5-one

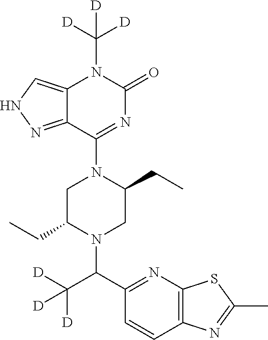 [SMILES: [2H]C([2H])([2H])C(c1ccc2nc(C)sc2n1)N1C[C@H](CC)N(c2nc(=O)n(C([2H])([2H])[2H])c3c[nH]nc23)C[C@H]1CC]

To a solution of 7-((2S,5R)-2,5-diethyl-4-(1-(2-methylthiazolo[5,4-b]pyridin-5-yl)ethyl-2,2,2-d₃)piperazin-1-yl)-4-(methyl-d3)-2-(tetrahydro-2H-pyran-2-yl)-2,4-dihydro-5H-pyrazolo[4,3-b]pyridin-5-one (90 mg, 0.16 mmol) in DCM (2 mL) was added TFA (6 mL). The resulted mixture was stirred at room temperature for 16 hours. The mixture was concentrated under reduced pressure to give titled compound (90 mg crude TFA salt). MS: M/e 472 (M+1)⁺.

### Step F: 2-(7-((2S,5R)-2,5-diethyl-4-(1-(2-methylthiazolo[5.4-b]pyridin-5-yl)ethyl-2.2.2-d3)apiperazin-1-yl)-4-(methyl-d3-5-oxo-4,5-dihydro-2H-pyrazolo[4,3-blyvridin-2-yl)acetonitrile

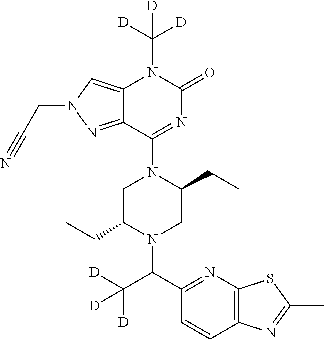 [SMILES: [2H]C([2H])([2H])C(c1ccc2nc(C)sc2n1)N1C[C@H](CC)N(c2nc(=O)n(C([2H])([2H])[2H])c3cn(CC#N)nc23)C[C@H]1CC]

To a mixture of 7-((2S,5R)-2,5-diethyl-4-(1-(2-methylthiazolo[5,4-b]pyridin-5-yl)ethyl-2,2,2-d₃)piperazin-1-yl)-4-(methyl-d₃)-2,4-dihydro-5H-pyrazolo[4,3-b]pyridin-5-one (90 mg, crude) and K₂CO₃ (223 mg, 1.62 mmol) in DMF (3 mL) was added 2-chloroacetonitrile (60 mg, 0.8 mmol). The reaction was stirred at room temperature for 16 hours. The reaction was diluted with water, extracted with EtOAc (60 mL×2), washed with brine, dried over Na₂SO₄, filtered, and concentrated. The residue was purified and separated into Compound A142a (14 mg, 17% for two steps) and Compound A142b (19 mg, 23% for two steps) by Prep-HPLC (Method A).

Compound A142a (the earlier peak): ¹HNMR (400 MHz, CD₃OD) δ 8.23 (d, J=8.8 Hz, 1H), 7.93 (s, 1H), 7.77 (d, J=8.8 Hz, 1H), 5.55 (s, 1H), 5.47 (s, 2H), 3.89 (s, 1H), 3.60-3.48 (m, 1H), 3.32-3.29 (m, 2H), 3.23-3.12 (m, 1H), 2.86 (s, 3H), 2.83-2.74 (m, 1H), 2.35-2.22 (m, 1H), 2.05-1.90 (m, 1H), 1.77-1.49 (m, 3H), 1.04 (t, J=7.2 Hz, 3H), 0.62 (t, J=7.2 Hz, 3H) ppm. MS: M/e 511 (M+1).

Compound A142b (the later peak): ¹HNMR (400 MHz, CD₃OD) 5 8.22 (d, J=8.4 Hz, 1H), 7.93 (s, 1H), 7.72 (d, J=8.0 Hz, 1H), 5.56 (s, 1H), 5.46 (s, 2H), 4.09 (s, 1H), 3.41-3.34 (m, 1H), 3.32-3.29 (m, 2H), 3.10-2.96 (m, 2H), 2.86 (s, 3H), 2,50-2.39 (m, 1H), 2.16-2.02 (m, 1H), 1.92-1.78 (m, 1H), 1.65-1.50 (m, 2H), 0.96 (t, J=7.2 Hz, 3H), 0.72 (t, J=7.6 Hz, 3H) ppm. MS: M/e 511 (M+1)⁺.

### Compound A143: 2-(7-((2S,5R)-2,5-diethyl-4-(1-(2-(methoxymethyl)thiazolo[5,4-b]pyridin-5-yl)ethyl)piperazin-1-yl)-4-methyl-5-oxo-4,5-dihydro-2H-pyrazolo[4,3-b]pyridin-2-yl)acetonitrile

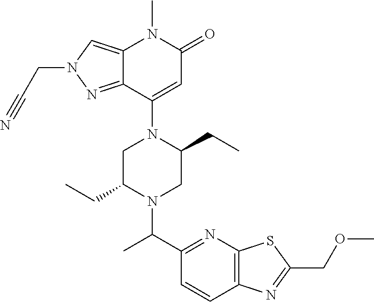 [SMILES: CC[C@H]1CN(C(C)c2ccc3nc(COC)sc3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (64 mg, 0.2 mmol) and 1-(2-(methoxymethyl)thiazolo[5,4-b]pyridin-5-yl)ethan-1-ol (45 mg, 0.2 mmol), (cyanomethyl) trimethyl phosphonium iodide (147 mg, 0.6 mmol) in CH₃CN (2 mL) was added DIPEA (129 mg, 1 mmol). The mixture was sealed in a bottle and heated at 100° C. for 16 hours. The mixture was cooled to room temperature, diluted with water, extracted with EtOAc (70 mL), washed with brine, dried over Na₂SO₄, filtered, and concentrated to dryness. The resulting residue was purified by Prep-TLC (EtOAc) and further separated into Compound A143a (21 mg, 19.6%) and Compound A143b (18 mg, 16.8%) by Prep-HPLC (Method A).

Compound A143a (the earlier peak): ¹H NMR (400 MHz, CD₃OD) δ 8.29 (d, J=8.4 Hz, 1H), 7.93 (s, 1H), 8.29 (d, .=8.8 Hz, 1H), 5.56 (s, 1H), 5.47 (s, 2H), 4.86 (s, 2H), 4.00-3.82 (m, 1H), 3.66-3.54 (m, 4H), 3.43 (s, 3H), 3.32-3.29 (m, 2H), 3.23-3.14 (m, 1H), 2.85-2.74 (m, 1H), 2.35-2.22 (m, 1H), 2.05-1.88 (m, 1H), 1.80-1.49 (m, 3H), 1.41 (d, J=6.8 Hz, 3H), 1.04 (t, J=7.6 Hz, 3H), 0.63 (t, J=7.6 Hz, 3H) ppm. MS: M/e 535 (M+1)⁺.

Compound A143b (the later peak): ¹H NMR (400 MHz, CD₃OD) δ 8.30 (d, J=8.8 Hz, 1H), 7.92 (s, 1H), 7.75 (d, J=8.4 Hz, 1H), 5.56 (s, 1H), 5.46 (s, 2H), 4.86 (s, 2H), 4.16-4.04 (m, 1H), 3.55 (s, 3H), 3.43 (s, 3H), 3.39-3.34 (m, 1H), 3.32-3.29 (m, 2H), 3.10-2.91 (m, 2H), 2,46-2.32 (m, 1H), 2.16-2.00 (m, 1H), 1.91-1.75 (m, 1H), 1.68-1.52 (m, 2H), 1.45 (d, J=6.4 Hz, 3H), 0.95 (t, J=7.6 Hz, 3H), 0.71 (t, J=7.6 Hz, 3H) ppm. MS: M/e 535 (M+1)⁺.

### Compound A144: 2-(7-((2S,5R)-4-(1-(2-(difluoromethyl)thieno[2,3-b]pyridin-6-yl)ethyl)-2,5-diethylpiperazin-1-yl)-4-methyl-5-oxo-4,5-dihydro-2H-pyrazolo[4,3-b]pyridin-2-yl)acetonitrile

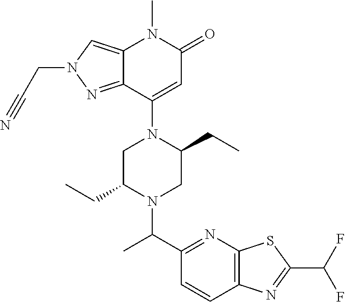 [SMILES: CC[C@H]1CN(C(C)c2ccc3nc(C(F)F)sc3n2)[C@H](CC)CN1c1cc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), 1-(2-(difluoromethyl)thieno[2,3-b]pyridin-6-yl)ethan-1-ol (34 mg, 0.15 mmol), (cyanomethyl)trimethylphosphonium iodide (120 mg, 0.5 mmol) and DIPEA (129 mg, 1 mmol) in MeCN (3 mL) was stirred at 100° C. for 16 hours. The resulted mixture was diluted with EtOAc (10 mL), washed with brine (5 mL×3), dried and concentrated. The resulting residue was purified by flash column chromatography to give the titled compound, which was separated into Compound A144a (9 mg, 3.5%) and Compound A144b (10 mg, 3.9%) by prep-HPLC (Method A).

Compound A144a (the first peak)). ¹H NMR (400 MHz, CD₃OD) δ 8.29 (d, J=8.4 Hz, 1H), 7.93 (s, 1H), 7.74 (d, J=8.4 Hz, 1H), 7.66 (s, 1H), 7.14 (t, J=55.1 Hz, 1H), 5.55 (s, 1H), 5.47 (s, 2H), 3.91-3.89 (m, 1H), 3.56-3.54 (m, 1H), 3.45 (s, 3H), 3.33-3.31 (m, 1H), 3.27-3.25 (m, 1H), 3.19-3.17 (m, 1H), 2.80-2.78 (m, 1H), 2.29-2.27 (m, 1H), 1.99-1.97 (m, 1H), 1.65-1.63 (m, 3H), 1.41 (d, J=6.7 Hz, 3H), 1.04 (t, J=7.3 Hz, 3H), 0.62 (t, J=7.4 Hz, 3H) ppm. MS: M/e 540 (M+1)⁺.

Compound A144b (the second peak)). ¹H NMR (400 MHz, CD₃OD) δ 8.30 (d, J=8.3 Hz, 1H), 7.92 (s, 1H), 7.69-7.67 (m, 2H), 7.14 (t, J=55.1 Hz, 1H), 5.56 (s, 1H), 5.46 (s, 2H), 4.07 (q, J=6.5 Hz, 1H), 3.43 (s, 3H), 3.37-3.35 (m, 2H), 3.27-3.25 (m, 1H), 3.00-2.98 (m 2H), 2,40-2.38 (m, 1H), 2.11-2.09 (m, 1H), 1.85-1.83 (m, 1H), 1.61-1.59 (m, 2H), 1.44 (d, J=6.6 Hz, 3H), 0.95 (t, 0.1=7.4 Hz, 3H), 0.71 (t, J=7.3 Hz, 3H) ppm. MS: M/e 540 (M+1)⁺.

### Compound A145: 2-(7-((2S,5R)-2,5-diethyl-4-(1-(2-(methoxymethyl)benzo[d]thiazol-6-yl)ethyl)piperazin-1-yl)-4-methyl-5-oxo-4,5-dihydro-2H-prazolo[4,3-b]pridin-2-yl)acetonitrile

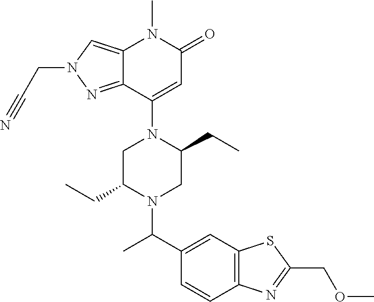 [SMILES: CC[C@H]1CN(C(C)c2ccc3nc(COC)sc3c2)[C@H](CC)CN1c1cc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (60 mg, 0.2 mmol), 1-(2-(methoxymethyl)benzo[d]thiazol-6-yl)ethan-1-ol (80 mg, 0.35 mmol), (cyanomethyl)trimethylphosphonium iodide (120 mg, 0.5 mmol) and DIPEA (129 mg, 1 mmol) in MeCN (3 mL) was stirred at 100° C. for 16 hours. The resulted mixture was diluted with EtOAc (10 mL), washed with brine (5 mL×3), dried and concentrated. The resulting residue was purified by flash column chromatography to give the titled compound, which was separated into Compound A145a (6 mg, 2.2%) and Compound A145b (7 mg, 2.6%) by Prep-HPLC (Method A).

Compound A145a (the first peak)). ¹H NMR (400 MHz, CD₃OD) δ 8.00 (s, 1H), 7.92 (d, J=7.3 Hz, 2H), 7.57 (d, J=7.6 Hz, 1H), 5.55 (s, 1H), 5.45 (s, 2H), 4.85 (s, 2H), 3.91 (d, J=6.5 Hz, 1H), 3.53 (s, 3H), 3.43 (s, 3H), 3.27-3.25 (m, 2H), 3.14-3.12 (m, 1H), 3.06-3.04 (m, 1H), 2.91-2.89 (m, 1H), 2,42-2,40 (m, 1H), 2.16-2.14 (m, 1H), 1.86-1.84 (m, 1H), 1.56-1.54 (m, 2H), 1.40 (d, J=6.4 Hz, 3H), 0.97 (t, J=7.4 Hz, 3H), 0.67 (t, .I=7.4 Hz, 3H) ppm. MS: We 534 (M+1)⁺.

Compound A145b (the second peak)). ¹H NMR (400 MHz, CD₃OD) δ 8.03 (s, 1H), 7.91-7.89 (m, 2H), 7.60 (d, .=8.5 Hz, 1H), 5.54 (s, 1H), 5.46 (s, 1H), 4.84 (s, 2H), 3.78-3.76 (m, 1H), 3.54-3.52 (m, 4H), 3.46-3.44 (m, 4H), 3.32-3.30 (m, 2H), 3.20-3.18 (m, 1H), 2.68-2.66 (m, 1H), 2.38-2.36 (m, 1H), 2.00-1.98 (m, 1H), 1.67-1.65 (m, 3H), 1.38 (d, J=6.5 Hz, 3H), 1.04 (s, 3H), 0.57 (s, 3H) ppm. MS: M/e 534 (M+1)⁺.

### Compound A146: 2-(7-((2S,5R)-4-(1-(2-cyclopropoxythiazolo[5,4-b] pyridin-5-yl)ethyl)-2,5-diethylpiperazin-1-yl)-4-methyl-5-oxo-4,5-dihydro-2H-pyrazolo[4,3-b]pyridin-2-yl)acetonitrile

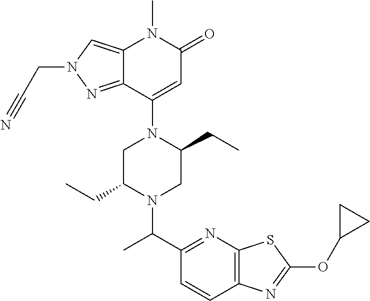 [SMILES: CC[C@H]1CN(C(C)c2ccc3nc(OC4CC4)sc3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(2-cyclopropoxythiazolo[5,4-b]pyridin-5-yl)ethan-1-ol (43 mg, 0.18 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduce pressure to afford crude product. The resulting residue was purified and separated into Compound A146a (20 mg) and Compound A146b (16 mg, 43%) by Prep-HPLC (Method A).

Compound A146a (the earlier peak): ¹H NMR (400 MHz, CD₃OD) δ 7.99 (d, J=8.4 Hz, 1H), 7.92 (s, 1H), 7.68 (d, J=8.4 Hz, 1H), 5.55 (s, 1H), 5.47 (s, 2H), 4.55-4.50 (m, 1H), 3.83 (d, J=6.4 Hz, 1H), 3.54 (d, J=11.7 Hz, 1H), 3.47 (s, 1H), 3.43 (s, 3H), 3.21-3.10 (m, 2H), 2.79-2.73 (m, 1H), 2.32-2.27 (m, J=11.3 Hz, 1H), 2.01-1.93 (m, 1H), 1.73-1.64 (m, 2H), 1.58-1.51 (m, 1H), 1.38 (d, J=6.6 Hz, 3H), 1.03 (t, J=7.3 Hz, 3H), 0.99-0.90 (m, J=12.8 Hz, 4H), 0.64 (t, J=7.3 Hz, 3H) ppm. MS: We 547 (M+1)⁺.

Compound A146b (the later peak): ¹H NMR (400 MHz, CD₃OD) δ 8.01 (d, J=8.3 Hz, 1H), 7.92 (s, 1H), 7.62 (d, J=8.4 Hz, 1H), 5.55 (s, 1H), 5.46 (s, 2H), 4.55-4.50 (m, 1H), 4.01 (d, J=6.3 Hz, 1H), 3.43 (s, 3H), 3.36-3.31 (m, 3H), 3.02-2.93 (m, 2H), 2,43-2.38 (m, 1H), 2.13-2.04 (m, 1H), 1.86-1.78 (m, 1H), 1.62-1.53 (m, 2H), 1.42 (d, J=6.6 Hz, 3H), 0.98-0.90 (m, 7H), 0.72 (t, J=7.3 Hz, 3H) ppm. MS: M/e 547 (M+1)⁺.

### Compound A147: 2-(7-((2S,5R)-2,5-diethyl-4-(1-(2-(2-fluoroethoxy) thiazolo[5,4-b]pyridin-5-yl)ethyl)piperazin-1-yl)-4-methyl-5-oxo-4,5-dihydro-2H-pyrazolo[4,3-b]pyridin-2-yl)acetonitrile

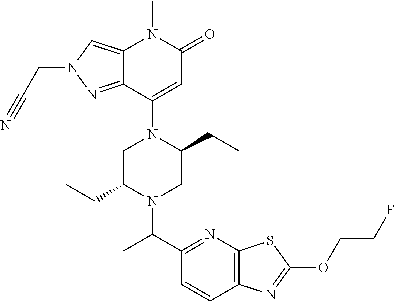 [SMILES: CC[C@H]1CN(C(C)c2ccc3nc(OCCF)sc3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.14 mmol) in CH₃CN (3 mL) and was added 1-(2-(2-fluoroethoxy)thiazolo[5,4-b]pyridin-5-yl)ethan-1-ol (44 mg, 0.18 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduce pressure to afford crude product. The resulting residue was purified and separated into Compound A147a (24 mg, 26%) and Compound A147b (18 mg, 24%) by Prep-HPLC (Method A).

Compound A147a (the earlier peak): ¹H NMR (400 MHz, CD₃OD) δ 7.98 (d, J=8.4 Hz, 1H), 7.92 (s, 1H), 7.60 (d, J=8.4 Hz, 1H), 5.55 (s, 1H), 5.46 (s, 2H), 4.77 (dd, J=20.9, 4.2 Hz, 2H), 4.69-4.46 (m, 1H), 4.00 (d, J=6.5 Hz, 1H), 3.43 (s, 3H), 3.37-3.31 (m, 4H), 3.03-2.92 (m, 2H), 2,43-2.37 (m, 1H), 2.13-2.04 (m, 1H), 1.86-1.78 (m, 1H), 1.61-1.51 (m, 2H), 1.42 (d, J=6.6 Hz, 3H), 0.95 (t, J=7.5 Hz, 3H), 0.72 (t, J=7.3 Hz, 3H) ppm. MS: M/e 553 (M+1)”.

Compound A147b (the later peak): ¹H NMR (400 MHz, CD₃OD) δ 7.97 (d, J=8.4 Hz, 1H), 7.92 (s, 1H), 7.66 (d, J=8.4 Hz, 1H), 5.55 (s, 1H), 5.47 (s, 2H), 4.85-4.80 (m, 1H), 4.80-4.70 (m, 2H), 3.82 (d, J=6.7 Hz, 1H), 3.53 (d, J=13.4 Hz, 1H), 3.43 (s, 3H), 3.35-3.31 (m, 3H), 3.15 (d, J=9.9 Hz, 1H), 2.76 (d, J=8.2 Hz, 1H), 2.30 (d, J=12.2 Hz, 1H), 2.02-1.92 (m, 1H), 1.73-1.63 (m, 2H), 1.58-1.51 (m, 1H), 1.38 (d, J=6.6 Hz, 3H), 1.03 (t, J=7.4 Hz, 3H), 0.63 (t, J=7.4 Hz, 3H) ppm. MS: M/e 553 (M+1)⁺.

### Compound A148: 2-(7-((2S,5R)-4-(1-(2-(difluoromethyl)thieno[2,3-b]pyridin-6-yl)ethyl)-2,5-diethylpiperazin-1-yl)-4-methyl-5-oxo-4,5-dihydro-2H-pyrazolo[4,3-d]pyrimidin-2-yl)acetonitrile

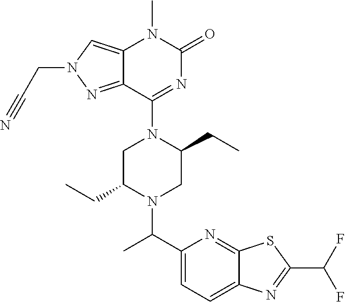 [SMILES: CC[C@H]1CN(C(C)c2ccc3nc(C(F)F)sc3n2)[C@H](CC)CN1c1nc(=O)n(C)c2cn(CC#N)nc12]

### Step A: methyl thienol2.3-b]pyridine-2-carboxylate

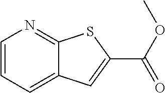

To a solution of 2-chloronicotinaldehyde (2.8 g, 20 mmol) in DMF (10 mL) was added methyl 2-mercaptoacetate (2.3 g, 22 mmol) and K₂CO₃ (4.2 g, 30 mmol) at room temperature and the mixture was stirred at 80° C. for 2 hours. The reaction mixture was poured into water (30 mL) and then extracted with EtOAc (30 mL×3). The organic layer was washed with brine (30 mL), dried and concentrated. The resulting residue was purified by flash column chromatography with 0-100% EA in PE to give the titled compound (3 g, 78.9%). MS: M/e 194 (M+1)⁺.

### Step B: thienol2.3-b]pyridin-2-vlmethanol

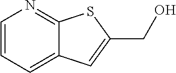 [SMILES: OCc1cc2cccnc2s1]

To a solution of methyl thieno[2,3-b]pyridine-2-carboxylate (2.6 g, 13.8 mmol) in THF (10 mL) at 0° C. was slowly added LiAIH4 (576 mg, 15.2 mmol). The reaction mixture was stirred at 0° C. for 0.5 hour. The reaction mixture was slowly added H₂O (0.6 mL), 15% NaOH aqueous solution (0.6 mL), and H₂O (1.8 mL). The mixture was filtered. The filtrate was concentrated in vacuo. The resulting residue was purified by flash column chromatography with 0-100% EA in PE to give the titled compound (2.2 g, 100%). MS: M/e 166 (M+1)⁺.

### Step C: thienol23-b]pyridine-2-carbaldehvde

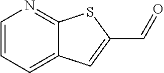 [SMILES: O=Cc1cc2cccnc2s1]

To a solution of thieno[2,3-b]pyridin-2-ylmethanol (1.8 g, 11 mmol) in DCM was added Des Martin reagent (6 g, 14.2 mmol) and the mixture was stirred at room temperature for overnight. The reaction mixture was added saturated NaHCO₃ aqueous solution (30 mL) and then extracted with EtOAc (30 mL×3). The organic layer was washed with brine (30 mL), dried and concentrated. The resulting residue was purified by flash column chromatography with 0-1⁰⁰⁰/ EA in PE to give the titled compound (1.45 g, 80.5%). MS: M/e 164 (M+1)⁺.

### Step D: 2-(difluoromethyl)thienol2.3-b]pyridine

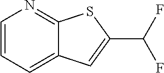 [SMILES: FC(F)c1cc2cccnc2s1]

To a solution of thieno[2,3-b]pyridine-2-carbaldehyde (1.45 g, 8.9 mmol) in DCM was added DAST (7.2 g, 44.5 mmol) and the mixture was stirred at room temperature for overnight. The reaction mixture was added saturated NaHCO₃ aqueous solution (30 mL) and then extracted with DCM (30 mL×3). The organic layer was washed with brine (30 mL), dried and concentrated. The resulting residue was purified by flash column chromatography with 0-100% EA in PE to give the titled compound (1 g, 60.9%). MS: M/e 186 (M+1)⁺.

### Step E: 2-(difluoromethyl)thienol23-bliyridine 7-oxide

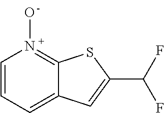

To a solution of 2-(difluoromethyl)thieno[2,3-b]pyridine (1 g, 5.3 mmol) in DCM was added m-CPBA (1.38 g, 8 mmol) and the mixture was stirred at room temperature for overnight. The reaction mixture was added saturated K₂O₃ aqueous solution (30 mL) and then extracted with DCM (30 mL×3). The organic layer was washed with brine (30 mL), dried and concentrated. The resulting residue was used directly in next step. MS: M/e 202 (M+1)⁺.

### Step F: 2-(difluoromethyl)thienol23-b]pyridine-6-carbonitrile

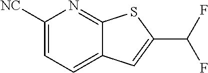 [SMILES: N#Cc1ccc2cc(C(F)F)sc2n1]

To the solution of 2-(difluoromethyl)thieno[2,3-b]pyridine 7-oxide from last step in DCM was added Et₃N (1.5 g, 15 mmol) and TMSCN (1.5 g, 15 mmol). The reaction mixture was stirred at 90° C. overnight. The reaction mixture was added H₂O (30 mL) and then extracted with EtOAc (30 mL×3). The organic layer was washed with brine (30 mL), dried and concentrated. The resulting residue was purified by flash column chromatography with 0-100% EA in PE to give the titled compound (700 mg, 63.6% for two steps). MS: M/e 211 (M+1)⁺.

### Step G: 2-(difluoromethyl)thienol23-b]pyridine-6-carbaldehvde

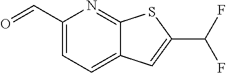 [SMILES: O=Cc1ccc2cc(C(F)F)sc2n1]

To a solution of 2-(difluoromethyl)thieno[2,3-b]pyridine-6-carbonitrile (416 mg, 1.98 mmol) in THF was added DABIL-H (1 M. 4 mmol, 4 mL) and the mixture was stirred at room temperature for 1 h. The reaction mixture was added 2N HCl solution (2 mL) and stirred at RT for 10 mins. The resulting solution was added saturated NaHCO₃ aqueous solution (30 mL) and then extracted with EtOAc (30 mL×3). The organic layer was washed with brine (30 mL), dried and concentrated. The resulting residue was purified by flash column chromatography with 0-100% EA in PE to give the titled compound (220 mg, 52.2%). MS: M/e 214 (M+1)⁺.

### Step H: 1-(2-(difluoromethyl)thienol23-b]pyridin-6-yl)ethan-1-ol

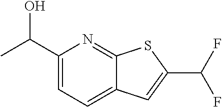

To a solution of 2-(difluoromethyl)thieno[2,3-b]pyridine-6-carbaldehyde (300 mg, 1.4 mmol) in THF was added CH₃MgBr (3 M. 1.69 mmol, 0.56 ml) at 0° C. and the mixture was stirred at 0° C. for 0.5 h. The reaction mixture was H₂O (30 mL) and then extracted with EtOAc (30 mL×3). The organic layer was washed with brine (30 mL), dried and concentrated. The resulting residue was purified by flash column chromatography with 0-100% EA in PE to give the titled compound (290 mg, 90%). MS: M/e 230 (M+1)”.

### Step I: tert-butyl (2S,5R)-4-(1-(2-(difluoromethyl)thienol2.3-b]pyridin-6-yl)ethyl)-2,5-diethylpiperazine-1-carboxylate

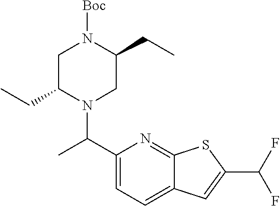 [SMILES: CC[C@H]1CN(C(C)c2ccc3cc(C(F)F)sc3n2)[C@H](CC)CN1C(=O)OC(C)(C)C]

A mixture of 1-(2-(difluoromethyl)thieno[2,3-b]pyridin-6-yl)ethan-1-ol (130 mg, 0.57 mmol), tert-butyl (2S,5R)-2,5-diethylpiperazine-1-carboxylate (137 mg, 0.57 mmol), (cyanomethyl)trimethylphosphonium iodide (361 mg, 1.5 mmol) and DIPEA (387 mg, 3 mmol) in MeCN (5 mL) was sealed and stirred at 100° C. for 16 hours. The resulted mixture was diluted with EtOAc (10 mL), washed with brine (5 mL×3), dried and concentrated. The resulting residue was purified by flash column chromatography to give the titled Compound (124 mg, 90%). MS: M/e 454 (M+1)⁺.

### Step J: 6-(1-((2R,5S)-2,5-diethylpiperazin-1-yl)ethyl)-2-(difluoromethyl)thienol2.3-b]pyridine

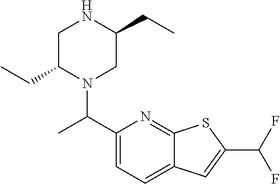 [SMILES: CC[C@H]1CN(C(C)c2ccc3cc(C(F)F)sc3n2)[C@H](CC)CN1]

To a solution of tert-butyl (2S,5R)-4-(1-(2-(difluoromethyl)thieno[2,3-b]pyridin-6-yl)ethyl)-2,5-diethylpiperazine-1-carboxylate (270 mg, 0.59 mmol) in DCM was added HCl/Dioxane (4N, 8 mmol, 2 ml) and the reaction mixture was stirred at rt for 15 mins. The reaction mixture was concentrated in vacuo. The residue was added saturated NaHCO₃ aqueous solution (10 ml) and then extracted with DCM (30 ml×3). The organic layer was washed with brine (30 ml), dried and concentrated in vacuo to give the titled compound (180 mg, 85.7%). MS: M/e 354 (M+1)⁺.

### Step K: 7-((2S,5R)₄-(1-(2-(difluoromethyl)thienol2.3-b]pyridin-6-yl)ethyl)-2,5-diethylpiperazin-1-yl)-4-methyl-2-(tetrahydro-2H-pyran-2-yl)-2,4-dihydro-5H-pyrazolo[4,3-d]pyrimidin-5-one

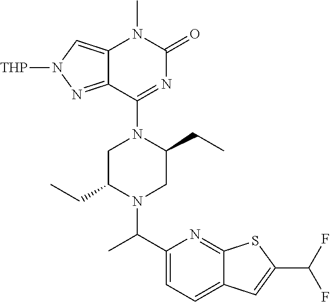

To a solution of 7-hydroxy-4-methyl-2-(tetrahydro-2H-pyran-2-yl)-2,4-dihydro-5H-pyrazolo[4,3-d]pyrimidin-5-one (64 mg, 0.256 mmol) in MeCN (3 ml) was added DIEA (66, 0.52 mmol) and POCl₃ (79 mg, 0.52 mmol). The reaction mixture was stirred at 80° C. for 1.5 hours. The mixture was added cold H₂O and extracted with EtOAc (30 ml). The organic layer was washed with brine (30 ml), dried and concentrated in vacuo. The residue was dissolved in MeCN (5 ml) and added 6-(1-((2R,5S)-2,5-diethylpiperazin-1-yl)ethyl)-2-(difluoromethyl)thieno[2,3-b]pyridine (180 mg, 0.51 mmol), DIPEA (66 mg, 0.52 mmol). The reaction mixture was stirred at 85° C. overnight. The mixture was added H₂O (10 ml) and extracted by EA (30 ml). The organic layer was washed with brine (30 ml), dried and concentrated in vacuo. The resulting residue was purified by flash column chromatography to give the titled compound (60 mg, 60%). MS: M/e 586 (M+1)⁺.

### Step L: 7-((2S,5R_{1.4}-(-(2-(difluoromethyl)thienol2.3-b]pyridin-6-yl)ethyl)-2,5-diethylpiperazin-1-yl-4-methyl-2,4-dihydro-5H-pyrazolo[4,3-d]pyrimidin-5-one

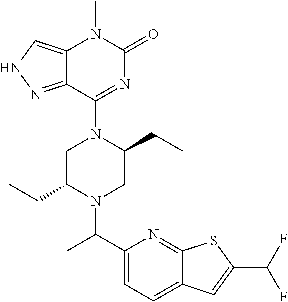 [SMILES: CC[C@H]1CN(C(C)c2ccc3cc(C(F)F)sc3n2)[C@H](CC)CN1c1nc(=O)n(C)c2c[nH]nc12]

To a solution of 7-((2S,5R)-4-(1-(2-(difluoromethyl)thieno[2,3-b]pyridin-6-yl)ethyl)-2,5-diethylpiperazin-1-yl)-4-methyl-2-(tetrahydro-2H-pyran-2-yl)-2,4-dihydro-5H-pyrazolo[4,3-d]pyrimidin-5-one (80 mg, 0.136 mmol) in DCM was added TFA (6 ml) and the reaction mixture was stirred at rt for overnight. The reaction mixture was concentrated in vacuo. The residue was added saturated NaHCO₃ aqueous solution (10 ml) and then extracted with DCM (30 ml×3). The organic layer was washed with brine (30 ml), dried and concentrated in vacuo to give the titled compound (65 mg, 95%). MS: M/e 502 (M+1)⁺.

### Step M: 2-(7-((2S,5R)-4-(1-(2-(difluoromethyl)thienol23-b]pyridin-6-yl)ethyl-2,5-diethylpiperazin-1-yl-4-methyl-5-oxo-4,5-dihydro-2H-pyrazolo[4,3-d]pyrimidin-2-yl)acetonitrile

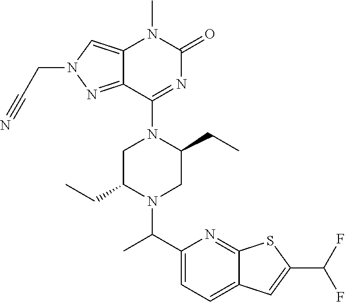 [SMILES: CC[C@H]1CN(C(C)c2ccc3cc(C(F)F)sc3n2)[C@H](CC)CN1c1nc(=O)n(C)c2cn(CC#N)nc12]

To a solution of 7-((2S,5R)-4-(1-(2-(difluoromethyl)thieno[2,3-b]pyridin-6-yl)ethyl)-2,5-diethylpiperazin-1-yl)-4-methyl-2,4-dihydro-5H-pyrazolo[4,3-d]pyrimidin-5-one (12 mg, 0.024 mmol) in DMF (5 ml) was added K₂CO₃ (13.9 mg, 0.1 mmol) and 2-chloroacetonitrile (5 mg, 0.05 mmol). The reaction mixture was stirred at rt for overnight. The reaction mixture was added H₂O (10 ml) and extracted with DCM (30 ml×3). The organic layer was washed with brine (30 ml), dried and concentrated in vacuo. The resulting residue was purified by flash column chromatography to give the titled compound, which was further separated into Compound A148a (4 mg, 31%) and Compound A148b (5 mg, 39%) by prep-HPLC (Method A).

Compound A148a (the earlier peak): ¹H NMR (400 MHz, CD₃OD) δ 8.29 (d, J=8.3 Hz, 1H), 7.91 (d, J=9.3 Hz, 1H), 7.74 (dd, J=8.3, 4.9 Hz, 1H), 7.66 (s, 1H), 7.14 (t, J=55.1 Hz, 1H), 5.81-5.75 (m, 0.5H), 5.54 (s, 1H), 5.50-5.47 (m, 1.5H), 5.26-5.14 (m, 0.5H), 5.00-4.98 (m, 0.5H), 3.90-3.86 (m, 1H), 3.39 (s, 3H), 3.35-3.15 (m, 2H), 2.78-2.68 (m, 1H), 2.29-2.27 (m, 1H), 2.10-1.90 (m, 2H), 1.54-1.49 (m, 2H), 1.43-1.39 (m, 3H), 1.09-0.98 (m, 3H), 0.68 (t, J=7.4 Hz, 3H) ppm. MS: M/e 541 (M+1)⁺.

Compound A148b (the later peak): ¹H NMR (400 MHz, CD₃OD) δ 8.30 (d, J=8.3 Hz, 1H), 7.89 (s, 1H), 7.69 (dd, J=14.7, 6.7 Hz, 2H), 7.14 (t, J=55.3 Hz, 1H), 5.79-5.70 (m, 0.5H), 5.62-5.54 (m, 1.5H), 5.46 (s, 11H), 5.22-5.15 (m, 0.5H), 5.06-4.98 (m, 0.5H), 4.06 (s, 1H), 3.58-3.54 (m, 0.5H), 3.44 (s, 3H), 3.15-3.12 (m, 0.5H), 3.06-3.03 (m, 1H), 2.96-2.98 (m, 1H), 2,43-2.39 (m, 1H), 2.12-2.05 (m, 1H), 1.67-1.60 (m, 2H), 1.49-1.43 (m, 4H), 0.95 (dd, J=13.2, 7.1 Hz, 3H), 0.71 (dt, J=25.0, 7.3 Hz, 3H) ppm. MS: M/e 541 (M+1)⁺.

### Compound A149: 2-(7-((2S,5R)-2,5-diethyl-4-(1-(2-methylimidazo 11.2-b]pyridazin-6-yl)ethyl)piperazin-1-yl)₄ methyl-5-oxo-4,5-dihydro-2H-pyrazolo[4,3-b]pyridin-2-yl)acetonitrile

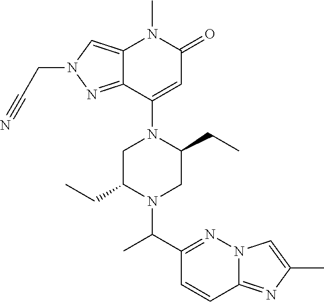 [SMILES: CC[C@H]1CN(C(C)c2ccc3nc(C)cn3n2)[C@H](CC)CN1c1cc(=O)n(C)c2cn(CC#N)nc12]

### Step A: 1-(2-methylimidazo[1,2-blpvridazin-6-vllethan-1-one

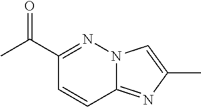

To a solution of 6-bromo-2-methylimidazo[1,2-b]pyridazine (212 mg, 1 mmol) in DMF (10 mL) was added tributyl(1-ethoxyvinyl)stannane (470 mg, 1.3 mmol) and Pd(PPh₂)₂Cl₂ (70 mg, 0.1 mmol). The reaction mixture was protected by N₂ atmosphere and stirred at 100° C. overnight. The mixture was cooled down to RT, 4M HCl in EA (2 mL) was added and the resulting mixture was stirred for one hour. The reaction was quenched with saturated NaHCO₃ solution to pH 2%=8, diluted with H₂O, extracted with EA (60 mL×2), washed with brine, dried over Na₂SO₄, filtered, and concentrated to dryness. The residue was purified by flash column chromatography to give the titled compound (145 mg, a little triphenylphosphine oxide contained, 82%).MS: M/e 176 (M+1)⁺.

### Step B: 1-(2-methylimidazo[1,2-blpvridazin-6-yl)ethan-1-ol

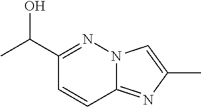

To a solution of 1-(2-methylimidazo[1,2-b]pyridazin-6-yl)ethan-1-one (140 mg, 0.8 mmol) in MeOH (5 mL) was added NaBH₄ (30 mg, 0.8 mmol). The resulting mixture was stirred at ice-bath for 30 mins. The reaction mixture was quenched with H₂O (20 mL) and extracted with EA (80 mL), washed with brine, dried over Na₂SO₄, filtered, and concentrated to dryness to give the titled compound (130 mg, 91%). MS: M/e 178 (M+1)⁺.

### Step C: 2-(7-((2S,5R-2,5-diethyl-4-(1-(2-methylimidazo [1,2-b]pyridazin-6-yl)ethyl)piperazin-1-yl)-4-methyl-5-oxo-4,5-dihydro-2H-pyrazolo[4,3-b]pyridin-2-yl)acetonitrile

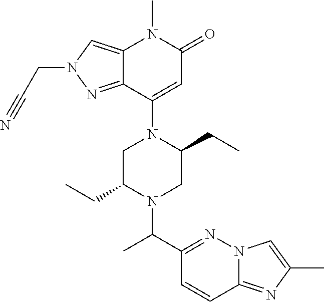 [SMILES: CC[C@H]1CN(C(C)c2ccc3nc(C)cn3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (66 mg, 0.2 mmol) and 1-(2-methylimidazo[1,2-b]pyridazin-6-yl)ethan-1-ol (50 mg, 0.3 mmol), (cyanomethyl) trimethyl phosphonium iodide (147 mg, 0.6 mmol) in CH₃CN (2 mL) was added DIPEA (129 mg, 1 mmol). The mixture was sealed in a bottle and heated at 100° C. for 16 hours. The mixture was cooled to room temperature, diluted with water, extracted with EtOAc (60 mL×2), washed with brine, dried over Na₂SO₄, filtered, and concentrated to dryness. The resulting residue was purified by prep-TLC (EtOAc) and further separated into Compound A149a (4 mg, 4%) and Compound A149b (6 mg, 6%) by Prep-HPLC (Method B).

Compound A149a (the earlier peak): ¹H NMR (400 MHz, CD₃OD) δ 7.92 (s, 1H), 7.86 (d, J=8.8 Hz, 1H), 7.82 (s, 1H), 7.45 (d, J=9.2 Hz, 1H), 5.55 (s, 1H), 5.47 (s, 2H), 3.90-3.75 (m, 1H), 3.57-3.46 (m, 1H), 3.42 (s, 3H), 3.32-3.29 (m, 2H), 3.17-3.06 (m, 1H), 2.84-2.72 (m, 1H), 2,44 (s, 3H), 2.35-2.26 (m, 1H), 1.96-1.80 (m, 1H), 1.76-1.60 (m, 2H), 1.57-1.45 (m, 1H), 1.41 (d, J=5.6 Hz, 3H), 1.02 (t, J=7.2 Hz, 3H), 0.64 (t, J=7.2 Hz, 3H) ppm. MS: M/e 488 (M+1)⁺.

Compound A149b (the later peak): ¹H NMR (400 MHz, CD₃OD) δ 7.92 (s, 1H), 7.87 (d, J=9.2 Hz, 1H), 7.83 (s, 1H), 7.40 (d, J=8.8 Hz, 1H), 5.56 (s, 1H), 5.45 (s, 2H), 4.06-3.95 (m, 1H), 3.42 (s, 3H), 3.38-3.33 (m, 1H), 3.32-3.29 (m, 2H), 2.99-2.92 (m, 2H), 2,50-2.38 (m, 4H), 2.15-1.95 (m, 1H), 1.86-1.70 (m, 1H), 1.67-1.52 (m, 2H), 1.43 (d, J=6.0 Hz, 3H), 0.90 (t, J=7.2 Hz, 3H), 0.76 (t, J=7.2 Hz, 3H) ppm. MS: M/e 488 (M+1)⁺.

### Compound A150: 2-(7-((2S,5R)-2,5-diethyl-4-(1-(2-methylimidazo[1,2-b]pyridazin-6-yl)ethyl)piperazin-1-yl)-4-methyl-5-oxo-4,5-dihydro-2H-pyrazolo[4,3-d]pyrimidin-2-yl)acetonitrile

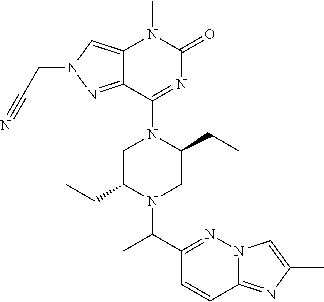 [SMILES: CC[C@H]1CN(C(C)c2ccc3nc(C)cn3n2)[C@H](CC)CN1c1nc(=O)n(C)c2cn(CC#N)nc12]

### Step A: tert-butyl (2S,5R)-2,5-diethyl-4-f4 (2-methylimidazo[1,2-blpvridazin-6-yl)ethyl)piperazine-1-carboxylate

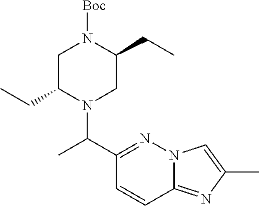 [SMILES: CC[C@H]1CN(C(C)c2ccc3nc(C)cn3n2)[C@H](CC)CN1C(=O)OC(C)(C)C]

To a solution of tert-butyl (2S,5R)-2,5-diethylpiperazine-1-carboxylate (242 mg, 1 mmol), 1-(2-methylimidazo[1,2-b]pyridazin-6-yl)ethan-1-ol (264 mg, 1.5 mmol) and DIPEA (645 mg, 5 mol) in CH₃CN (6 ml ) was added (cyanomethyl)trimethyl phosphonium iodide (730 mg, 3 mmol). The mixture was stirred at 103° C. in a sealed bottle for 16 hours. The reaction was cooled to room temperature, diluted by adding water, extracted with EtOAc (80 mL×2), washed with brine, dried over Na₂SO₄, filtered and concentrated to dryness. The residue was purified by flash column chromatography to give the titled compound (320 mg, 80%). MS: M/e 402 (M+1)⁺.

### Step B: 6-(1-((2R,5S)-2,5-diethylpiperazin-1-yl)ethyl)-2-methylimidazo[1,2-blDyridazine

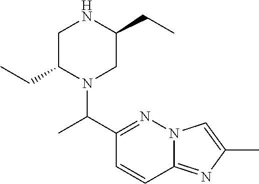 [SMILES: CC[C@H]1CN(C(C)c2ccc3nc(C)cn3n2)[C@H](CC)CN1]

To a solution of tert-butyl (2S,5R)-2,5-diethyl-4-(1-(2-methylimidazo[1,2-b]pyridazin-6-yl)ethyl)piperazine-1-carboxylate (320 mg, 0.8 mmol) in DCM (10 mL) was added TMSOTf (355 mg, 1.6 mmol). The resulted mixture was stirred at room temperature for 16 hours. The reaction mixture was quenched with aq. NaHCO₃, then extracted with CH₂Cl₂/IPA (3/1, 50 mL x 3). The combined organic layers were dried over Na₂SO₄, concentrated to give the titled compound (224 mg, 93%). MS: M/e 302 (M+1)⁺.

### Step C: 7-((2S,5R)-2,5-diethyl-4-(1-(2-methylimidazo[1,2-blpvridazin-6-yl)ethyl)piperazin-1-yl-4-methyl-2-(tetrahydro-2H-pyran-2-yl)-2,4-dihydro-5H-pyrazolo[4,3-d]pyrimidin-5-one

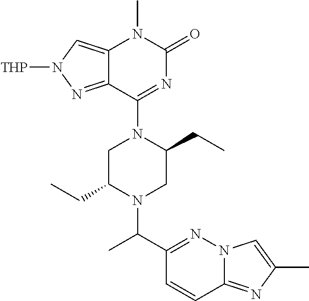

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5) in CH₃CN (3 mL) was added DIPEA (129 mg, 1 mmol), followed by POCl₃ (153 mg, 1 mmol) and catalyst amount of DMF. The reaction was stirred at 80° C. for 2 hours. The reaction was cooled to room temperature, quenched with ice-water, extracted with EtOAc (70 mL), washed with brine, dried over Na₂SO₄, filtered, and concentrated. The above residue was dissolved in CH₃CN (5 mL). 6-(1-((2R,5S)-2,5-diethylpiperazin-1-yl)ethyl)-2-methylimidazo[1,2-b]pyridazine (100 mg, 0.3 mmol,) and DIPEA (129 mg, 1 mmol) were added. The resulting mixture was heated at 85° C. for 16 hours. The reaction was quenched with water, extracted with EtOAc (80 mL), washed with brine, dried over Na₂SO₄, filtered, and concentrated. The residue was purified by flash column chromatography to give the titled compound (40 mg, 15% for two steps). MS: M/e 534 (M+1)⁺.

### Step D: 7-((2S,5R)-25-diethyl-4-(1-(2-methylimidazo[1,2-blovridazin-6-yl)ethyl)piperazin-1-yl)-4-methyl-2,4-dihydro-5H-pyrazolo[4,3-d]pyrimidin-5-one

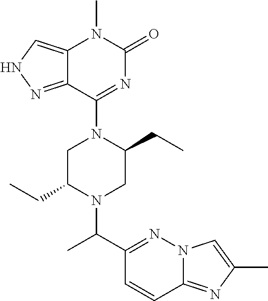 [SMILES: CC[C@H]1CN(C(C)c2ccc3nc(C)cn3n2)[C@H](CC)CN1c1nc(=O)n(C)c2c[nH]nc12]

To a solution of 7-((2S,5R)-2,5-diethyl-4-(1-(2-methylimidazo[1,2-b]pyridazin-6-yl)ethyl)piperazin-1-yl)-4-methyl-2-(tetrahydro-2H-pyran-2-yl)-2,4-dihydro-5H-pyrazolo[4,3-d]pyrimidin-5-one (40 mg, 0.075 mmol) in DCM (1 mL) was added TFA (3 mL). The resulted mixture was stirred at room temperature for 16 h. The mixture was concentrated under reduced pressure to give the titled compound (crude). MS: M/e 450 (M+1)⁺.

### Step E: 2-(7-((2S,5R)-2,5-diethyl-4-(1-(2-methylimidazo[1,2-b]pyridazin-6-yl)ethyl)piperazin-1-yl)-4-methyl-5-oxo-4,5-dihydro-2H-pyrazolo[4,3-d]pyrimidin-2-yl)acetonitrile

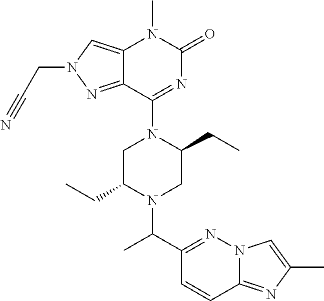 [SMILES: CC[C@H]1CN(C(C)c2ccc3nc(C)cn3n2)[C@H](CC)CN1c1nc(=O)n(C)c2cn(CC#N)nc12]

To a mixture of 7-((2S,5R)-2,5-diethyl-4-(1-(2-methylimidazo[1,2-b]pyridazin-6-yl)ethyl)piperazin-1-yl)-4-methyl-2,4-dihydro-5H-pyrazolo[4,3-d]pyrimidin-5-one (30 mg, crude) and K₂CO₃ (103 mg, 0.75 mmol) in DMF (2 mL) was added 2-chloroacetonitrile (30 mg, 0.375 mmol). The reaction was stirred at room temperature for 16 hours. The reaction was diluted with water, extracted with EtOAc (80 mL), washed with brine, dried over Na₂SO₄, filtered, and concentrated. The resulting residue was purified by Prep-TLC(EtOAc) and further separated into Compound A150a (4 mg, 10% for two steps) and Compound A150b (6 mg, 16% for two steps) by Prep-HPLC (Method A).

Compound A150a (the earlier peak): ¹HNMR (400 MHz, CD₃OD) δ 7.91 (d, J=6.8 Hz, 1H), 7.87 (d, J=9.6 Hz, 1H), 7.82 (s, 1H), 7.46 (d, J=9.2 Hz, 1H), 5.83-5.75 (m, 0.5H), 5.57-5.43 (m, 2,5H), 5.27-5.16 (m, 0.5H), 5.04-4.92 (m, 0.5H), 4.63-4.57 (m, 0.5H), 3.89-3.70 (m, 1.5H), 3.41 (s, 3H), 3.26-3.10 (m, 1H), 2.87-2.64 (m, 1H), 2,45 (s, 3H), 2.36-2.22 (m, 1H), 2.05-1.76 (m, 2H), 1.60-1.46 (m, 2H), 1.41 (d, J=6.4 Hz, 3H), 1.10-0.90 (m, 3H), 0.74-0.64 (m, 3H) ppm. MS: M/e 489 (M+1)⁺.

Compound A150b (the later peak): ¹HNMR (400 MHz, CD₃OD) 5 7.96-7.80 (m, 3H), 7.42 (d, J=8.8 Hz, 1H), 5.75-5.64 (m, 0.5H), 5.58-5.43 (m, 2,5H), 5.24-5.12 (m, 0.5H), 5.04-4.96 (m, 0.5H), 4.06-3.92 (m, 1H), 3.60-3.51 (m, 0.5H), 3.40 (s, 3H), 3.18-3.10 (m, 0.5H), 3.01-2.80 (m, 2H), 2,55-2,40 (m, 4H), 2.15-1.85 (m, 2H), 1.70-1.35 (m, 5H), 0.99-0.85 (m, 3H), 0.84-0.64 (m, 3H) ppm. MS: M/e 489 (M+1)⁺.

### Compound A151: 2-(7-((2S,5R)-2,5-diethyl-4-(1-(3-methylpyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile

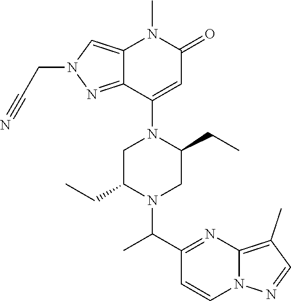 [SMILES: CC[C@H]1CN(C(C)c2ccn3ncc(C)c3n2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (92.7 mg, 0.28 mmol), 1-(3-methylpyrazolo[1,5-a]pyrimidin-5-yl)ethan-1-ol (100 mg, 0.56 mmol), (cyanomethyl)trimethylphosphonium iodide (206 mg, 0.85 mmol) and DIPEA (364.4 mg, 2.82 mmol) in CH₃CN (2 mL) was stirred at 100° C. overnight. The reaction was diluted with EtOAc (10 mL) and washed with brine (10 mL). The organic layer was concentrated under reduced pressure. The resulting residue was purified by flash column chromatography with 0-10% MeOH in DCM and further separated into Compound A151a (20.23 mg, 14.7%) and Compound A151b (25.4 mg, 18.4%) by Prep-HPLC (Method A).

Compound A151a (the earlier peak): 1H NMR (399 MHz, cd3od) δ 8.76 (d, J=7.3 Hz, 1H), 7.96 (s, 1H), 7.94 (s, 1H), 7.22 (d, J=7.3 Hz, 1H), 5.57 (s, 1H), 5.48 (s, 2H), 5.22-4.90 (m, 1H), 4.74-4.01 (m, 1H), 3.80 (q, J=6.7 Hz, 1H), 3.55 (d, J=12.0 Hz, 1H), 3.44 (s, 3H), 3.15 (d, J=10.7 Hz, 1H), 2.81 (dd, J=10.7, 3.7 Hz, 1H), 2.35 (s, 3H), 2.32 (d, J=13.1 Hz, 1H), 2.03-1.89 (m, 1H), 1.82-1.64 (m, 2H), 1.59-1.48 (m, 1H), 1.40 (d, J=6.7 Hz, 3H), 1.04 (t, J=7.4 Hz, 3H), 0.70 (t, J=7.4 Hz, 3H) ppm. MS: M/e 488 (M+1)⁺.

Compound A151b (the later peak): 1H NMR (399 MHz, cd3od) 8 8.75 (d, J=7.3 Hz, 1H), 7.96 (s, 1H), 7.93 (s, 1H), 7.17 (d, J=7.3 Hz, 1H), 5.57 (s, 1H), 5.47 (s, 2H), 5.15-4.90 (m, 1H), 4.85-4.15 (m, 1H), 4.00 (q, J=6.7 Hz, 1H), 3.44 (s, 3H), 3.38 (d, J=10.8 Hz, 1H), 3.05-2.92 (m, 2H), 2,46 (d, J=8.8 Hz, 1H), 2.35 (s, 3H), 2.16-2.03 (m, 1H), 1.90-1.78 (m, 1H), 1.70-1.56 (m, 2H), 1.43 (d, J=6.7 Hz, 3H), 0.94 (t, J=7.4 Hz, 3H), 0.77 (t, J=7.4 Hz, 3H) ppm. MS: M/e 488 (M+1)⁺.

### Compound A152: 2-(7-((2S,5R)-2,5-diethyl-4-(1-(2-methylpyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile

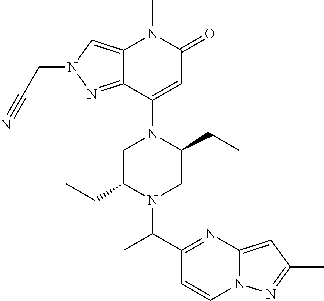 [SMILES: CC[C@H]1CN(C(C)c2ccn3nc(C)cc3n2)[C@H](CC)CN1c1cc(=O)n(C)c2cn(CC#N)nc12]

### Step A: 1-(2-methylpyrazolo[1,5-a]pyrimidin-5-yl)ethan-1-ol

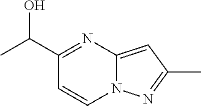

To a solution of 1-(2-methylpyrazolo[1,5-a]pyrimidin-5-yl)ethan-1-one (0.18 g, 0.73 mmol) in MeOH (5 mL) was added NaBH₄ (23 mg, 0.56 mmol) at RT and the resulting mixture was stirred at RT for 5 min. The reaction mixture was diluted with DCM and washed with water, dried over Na₂SO₄, concentrated to give the titled compound (170 mg, 85%). MS: M/e 178 (M+1)⁺.

### Step B: 2-(7-((2S,5R)-2,5-diethyl-4-(1-(2-methylpyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile

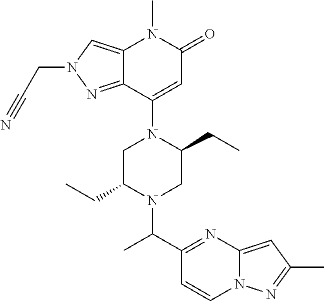 [SMILES: CC[C@H]1CN(C(C)c2ccn3nc(C)cc3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (66 mg, 0.2 mmol) and 1-(2-methylpyrazolo[1,5-a]pyrimidin-5-yl)ethan-1-ol (70 mg, 0.4 mmol), (cyanomethyl) trimethyl phosphonium iodide (147 mg, 0.6 mmol) in CH₃CN (2 mL) was added DIPEA (129 mg, 1 mmol). The mixture was sealed in a bottle and heated at 100° C. for 16 hours. The mixture was cooled to room temperature, diluted with water, extracted with EtOAc (70 mL×2), washed with brine, dried over Na₂SO₄, filtered, and concentrated to dryness. The resulting residue was purified by prep-TLC (EtOAc) and further separated into Compound A152a (9 mg, 9.2%) and Compound A152b (11 mg, 11.3%) by Prep-HPLC (Method A).

Compound A152a (the earlier peak): ¹H NMR (400 MHz, CD₃OD) δ 8.74 (d, J=7.2 Hz, 1H), 7.93 (s, 1H), 7.20 (d, J=7.2 Hz, 1H), 6.41 (s, 1H), 5.57 (s, 1H), 5.47 (s, 2H), 5.13-5.00 (m, 1H), 4.63-4.59 (m, 1H), 3.81-3.66 (m, 1H), 3.59-3.49 (m, 1H), 3.42 (s, 3H), 3.18-3.07 (m, 1H), 2.85-2.75 (m, 1H), 2,47 (s, 3H), 2.35-2.26 (m, 1H), 2.02-1.85 (m, 1H), 1.82-1.60 (m, 2H), 1.58-1.46 (m, 1H), 1.38 (d, J=6.4 Hz, 3H), 1.03 (t, J=7.2 Hz, 3H), 0.69 (t, J=7.2 Hz, 3H) ppm. MS: M/e 488 (M+1)⁺.

Compound A152b (the later peak): ¹H NMR (400 MHz, CD₃OD) δ 8.73 (d, J=7.2 Hz, 1H), 7.93 (s, 1H), 7.15 (d, J=7.2 Hz, 1H), 6.42 (s, 1H), 5.57 (s, 1H), 5.46 (s, 2H), 5.13-5.00 (m, 1H), 4.63-4.59 (m, 1H), 3.97-3.86 (m, 1H), 3.43 (s, 3H), 3.41-3.33 (m, 1H), 3.04-2.89 (m, 2H), 2,50-2.38 (m, 4H), 2.17-2.00 (m, 1H), 1.89-1.76 (m, 1H), 1.66-1.52 (m, 2H), 1.40 (d, J=6.8 Hz, 3H), 0.94 (t, J=7.2 Hz, 3H), 0.76 (t, J=7.2 Hz, 3H) ppm. MS: M/e 488 (M+1)⁺.

### Compound A153: 2-(7-((2S,5R)-2,5-diethyl-4-(1-(2-ethylimidazo [1,2-b]pyridazin-6-yl)ethyl)piperazin-1-yl)-4-methyl-5-oxo-4,5-dihydro-2H-pyrazolo[4,3-b]pyridin-2-yl)acetonitrile

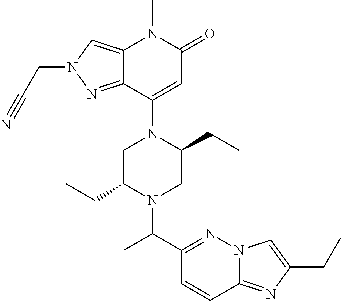 [SMILES: CCc1cn2nc(C(C)N3C[C@H](CC)N(c4cc(=O)n(C)c5cn(CC#N)nc45)C[C@H]3CC)ccc2n1]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (98 mg, 0.3 mmol) and 1-(2-ethylimidazo[1,2-b]pyridazin-6-yl)ethan-1-ol (79 mg, 0.45 mmol), (cyanomethyl) trimethyl phosphonium iodide (220 mg, 0.9 mmol) in CH₃CN (3 mL) was added DIPEA (193 mg, 1.5 mmol). The mixture was sealed in a bottle and heated at 100° C. for 16 hours. The mixture was cooled to room temperature, diluted with water, extracted with EtOAc (70 mL×3), washed with brine, dried over Na₂SO₄, filtered, and concentrated to dryness. The resulting residue was purified by prep-TLC (EtOAc) and further separated into Compound A153a (11 mg, 7.3%) and Compound A153b (15 mg, 10%) by Prep-HPLC (Method A).

Compound A153a (the earlier peak): ¹H NMR (400 MHz, CD₃OD) δ 7.94 (s, 1H), 7.87 (d, J=9.2 Hz, 1H), 7.84 (s, 1H), 7.46 (d, J=9.6 Hz, 1H), 5.55 (s, 1H), 5.47 (s, 2H), 5.14-5.00 (m, 1H), 4.65-4.59 (m, 1H), 3.87-3.79 (m, 1H), 3.58-3.48 (m, 1H), 3.43 (s, 3H), 3.18-3.10 (m, 1H), 2.87-2.74 (m, 3H), 2.36-2.25 (m, 1H), 1.95-1.84 (m, 1H), 1.77-1.62 (m, 2H), 1.60-1.46 (m, 1H), 1.41 (d, J=6.8 Hz, 3H), 1.35 (t, J=7.2 Hz, 3H), 1.03 (t, J=7.2 Hz, 3H), 0.65 (t, J=7.6 Hz, 3H) ppm. MS: M/e 502 (M+1)⁺.

Compound A153b (the later peak): ¹H NMR (400 MHz, CD₃OD) δ 7.93 (s, 1H), 7.89 (d, J=9.6 Hz, 1H), 7.86 (s, 1H), 7.41 (d, J=9.2 Hz, 1H), 5.56 (s, 1H), 5.46 (s, 2H), 5.14-5.00 (m, 1H), 4.65-4.59 (m, 1H), 4.05-3.97 (m, 1H), 3.43 (s, 3H), 3.39-3.33 (m, 1H), 3.01-2.93 (m, 2H), 2.87-2.76 (m, 2H), 2,48-2.38 (m, 1H), 2.10-1.96 (m, 1H), 1.86-1.74 (m, 1H), 1.66-1.56 (m, 2H), 1.44 (d, 0.1=7.2 Hz, 3H), 1.35 (t, J=7.6 Hz, 3H), 0.91 (t, J=7.6 Hz, 3H), 0.77 (t, J=7.6 Hz, 3H) ppm. MS: M/e 502 (M+1)⁺.

### Compound A154: 2-(7-((2S,5R)-2,5-diethyl-4-(1-(pyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile

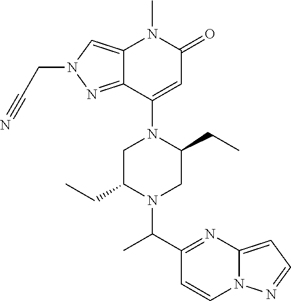 [SMILES: CC[C@H]1CN(C(C)c2ccn3nccc3n2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (100 mg, 0.30 mmol), 1-(pyrazolo[1,5-a]pyrimidin-5-yl)ethan-1-ol (99.4 mg, 0.61 mmol), (cyanomethyl)trimethylphosphonium iodide (222 mg, 0.91 mmol) and DIPEA (393 mg, 3.05 mmol) in CH₃CN (2 mL) was stirred at 100° C. overnight. The reaction was diluted with EtOAc (10 mL) and washed with brine (10 mL). The organic layer was concentrated under reduced pressure. The resulting residue was purified by flash column chromatography with 0-5% MeOH in DCM and further separated into Compound A154a (30.5 mg, 21%) and Compound A154b (37.5 mg, 26%) by Prep-HPLC (Method A).

Compound A154a (the earlier peak): ¹H NMR (400 MHz, CD₃OD) δ 8.88 (d, J=7.3 Hz, 1H), 8.13 (d, 0.1=2.3 Hz, 1H), 7.94 (s, 1H), 7.30 (d, .=7.3 Hz, 1H), 6.61 (d, J=2.2 Hz, 1H), 5.57 (s, 1H), 5.48 (s, 2H), 4.984.91 (m, 2H), 3.78 (q, J=6.7 Hz, 1H), 3.55 (d, J=13.2 Hz, 1H), 3.44 (s, 3H), 3.15 (d, J=10.6 Hz, 1H), 2.82 (d, J=8.9 Hz, 1H), 2.32 (d, J=12.1 Hz, 1H), 2.02-1.88 (m, 1H), 1.83-1.64 (m, 2H), 1.59-1.47 (m, 1H), 1.40 (d, J=6.7 Hz, 3H), 1.03 (t, J=7.3 Hz, 3H), 0.70 (t, J=7.4 Hz, 3H) ppm. MS: M/e 474 (M+1)⁺.

Compound A154b (the later peak): ¹H NMR (400 MHz, CD₃OD) δ 8.87 (d, J=7.3 Hz, 1H), 8.13 (d, J=2.3 Hz, 1H), 7.93 (s, 1H), 7.25 (d, J=7.3 Hz, 1H), 6.63 (d, J=2.3 Hz, 1H), 5.57 (s, 1H), 5.47 (s, 2H), 4.96-4.91 (m, 2H), 3.98 (q, J=6.6 Hz, 1H), 3.43 (s, 3H), 3.38 (d, J=10.7 Hz, 1H), 3.05-2.92 (m, 2H), 2,50-2,43 (m, 1H), 2.17-2.04 (m, 1H), 1.90-1.78 (m, 1H), 1.68-1.57 (m, 2H), 1.42 (d, J=6.6 Hz, 3H), 0.94 (t, J=7.4 Hz, 3H), 0.77 (t, J=7.4 Hz, 3H) ppm. MS: M/e 474 (M+1)⁺.

### Compound A155: 2-(7-((2S,5R)-2,5-dimethyl-4-(1-(2-methylthieno[3,2-b]pyridin-5-yl)ethyl)piperazin-1-yl)-4-methyl-5-oxo-4,5-dihydro-2H-pyrazolo[4,3-b]pyridin-2-yl)acetonitrile

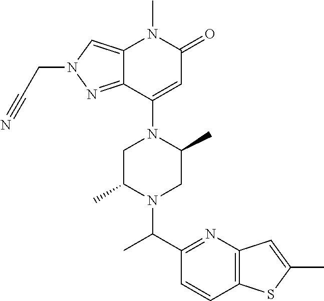 [SMILES: Cc1cc2nc(C(C)N3C[C@H](C)N(c4cc(=O)n(C)c5cn(CC#N)nc45)C[C@H]3C)ccc2s1]

### Step A: 2-methylthienol3.2-b]pyridine

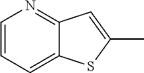

A mixture of 2-chlorothieno[3,2-b]pyridine (507 mg, 3 mmol), trimethylboroxine (3.5M in THF,1.3 mL, 4.5 mmol), potassium carbonate (621 mg, 4.5 mmol), and tetrakis(triphenylphosphine)palladium (346 mg, 0.3 mmol) in DMF (3 mL) was stirred at 130° C. for 1 hour at microwave conditions, then cooled to RT. The reaction was quenched with water(20 mL),extracted with EA (10 mL×2), dried over Na₂SO₄ and concentrated to dryness. The resulting residue was purified by flash column chromatography with 0-50/a EA in PE to give the titled compound (190 mg, 42%). MS: We 150 (M+1)⁺.

### Step B: 2-methylthienol3.2-b]pyridine 4-oxide

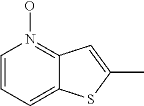

A solution of 2-methylthieno[3,2-b]pyridine (190 mg, 1,27 mmol) and m-CPBA (440 mg, 2,55 mmol) in DCM (5 mL) was stirred at RT for 2 hours. The reaction was washed with brine (10 mL×2), dried over Na₂SO₄ and concentrated to dryness. The resulting residue was purified by flash column chromatography with 0-10% MeOH in DCM to give the titled compound (140 mg, 67%). MS: M/e 166 (M+1)⁺.

### Step C: 2-methylthienol3.2-b]pyridine-5-carbonitrile

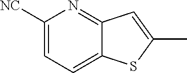

A solution of 2-methylthieno[3,2-b]pyridine 4-oxide (140 mg, 0.85 mmol), TMSCN (251 mg, 2,54 mmol), dimethylcarbamic chloride (275 mg, 2,54 mmol) and TEA (256 mg, 2,54 mmol) in MeCN (3 mL) was stirred at 75° C. overnight. The solution was diluted with EA (15 mL), washed with brine (10 mL×2), dried over Na₂SO₄ and concentrated to dryness. The resulting residue was purified by flash column chromatography with 0-50% EA in PE to give the titled compound (90 mg, 61%). MS: M/e 175 (M+1)⁺.

### Step D: 1-(2-methylthienol3.2-b]pyridin-5-yl)ethan-1-one

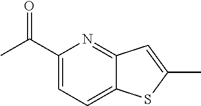

A solution of 2-methylthieno[3,2-b]pyridine-5-carbonitrile (90 mg, 0.52 mmol), MeMgBr (3M, 0.35 mL, 1.03 mmol) in THF (4 mL) under N₂ was stirred at RT overnight. Quench the reaction mixture by addition of saturated ammonium chloride solution (20 mL), extracted with EA (10 mL), washed with brine (10 mL×2), dried over Na₂SO₄ and concentrated to dryness. The resulting residue was purified by flash column chromatography with 0-10% EA in PE to give the titled compound (50 mg, 50%). MS: M/e 192 (M+1)⁺.

### Step E: 1-(2-methylthienol3.2-b]pyridin-5-yl)ethan-1-ol

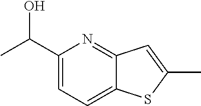

A solution of 1-(2-methylthieno[3,2-b]pyridin-5-yl)ethan-1-one (30 mg, 0.16 mmol) and NaBH₄ (4 mg, 0.11 mmol) in MeOH (2 mL) was stirred at RT for 30 min. The solution was diluted with EA (10 mL), washed with brine (10 mL×2), dried over Na₂SO₄ and concentrated to dryness to give the titled compound (30 mg, crude), which was used directly for the next step without further purification. MS: We 194 (M+1)”.

### Step F: 2-(7-((2S,5R)-2,5-dimethyl-4-(1-(2-methylthienol3.2-b]pyridin-5-yl)ethyl)piperazin-1-yl)-4-methyl-5-oxo-4,5-dihydro-2H-pyrazolo[4,3-b]pyridin-2-yl)acetonitrile

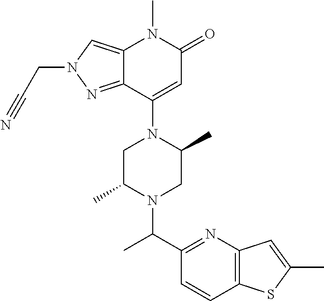 [SMILES: Cc1cc2nc(C(C)N3C[C@H](C)N(c4cc(=O)n(C)c5cn(CC#N)nc45)C[C@H]3C)ccc2s1]

A solution of 2-(7-((2S,5R)-2,5-dimethylpiperazin-1-yl)-4-methyl-5-oxo-4,5-dihydro-2H-pyrazolo[4,3-b]pyridin-2-yl)acetonitrile (117 mg, 0.39 mmol), 1-(2-methylthieno[3,2-b]pyridin-5-yl)ethan-1-ol (150 mg, 0.78 mmol), (cyanomethyl) trimethylphosphonium iodide (284 mg, 1.17 mmol) and DIPEA (503 mg, 3.90 mmol) in CH₃CN (2 mL) was stirred at 100° C. overnight. The reaction was diluted with EtOAc (10 mL) and washed with brine (10 mL). The organic layer was concentrated under reduced pressure. The resulting residue was purified by flash column chromatography with 0-10% MeOH in DCM and futher separated Compound A155a (32 mg, 18%) and Compound A155b (30 mg, 16%) by Prep-HPLC (Method B).

Compound A155a (the earlier peak): ¹H NMR (400 MHz, CD₃OD) δ 8.22 (d, J=8.4 Hz, 1H), 7.90 (s, 1H), 7.53 (d, J=8.4 Hz, 1H), 7.17 (s, 1H), 5.54 (s, 1H), 5.44 (s, 2H), 4.96-4.88 (m, 1H), 4.364.16 (m, 1H), 3.88 (q, J=6.6 Hz, 1H), 3.45 (dd, J=11.5, 3.7 Hz, 1H), 3.41 (s, 3H), 3.03 (dd, J=11.5, 3.7 Hz, 1H), 2.92-2.81 (m, 2H), 2.64 (s, 3H), 1.40 (d, J=6.5 Hz, 3H), 1.36 (d, J=6.6 Hz, 3H), 1.02 (d, J=6.5 Hz, 3H) ppm. MS: M/e 476 (M+1)⁺.

Compound A155b (the later peak): ¹H NMR (400 MHz, CD₃OD) δ 8.20 (d, J=8.4 Hz, 1H), 7.90 (s, 1H), 7.55 (d, J=8.5 Hz, 1H), 7.14 (s, 1H), 5.54 (s, 1H), 5.44 (s, 2H), 4.774.41 (m, 2H), 3.70 (q, J=6.7 Hz, 1H), 3.63 (d, J=12.7 Hz, 2H), 3.40 (s, 3H), 2.86 (dd, J=11.9, 3.6 Hz, 1H), 2.63 (s, 3H), 2.09 (d, J=11.9 Hz, 1H), 1.40 (d, J=6.6 Hz, 3H), 1,21 (d, J=6.6 Hz, 3H), 1.15 (d, J=6.5 Hz, 3H) ppm. MS: M/e 476 (M+1)⁺.

### Compound A156: 2-(7-((2S,5R)-4-(1-(2,3-dimethylpyrazolo[1,5-a]pyrimidin-5-yl)ethyl)-2,5-diethylpiperazin-1-yl)-4-methyl-5-oxo-4,5-dihydro-2H-pyrazolo[4,3-b]pyridin-2-yl)acetonitrile

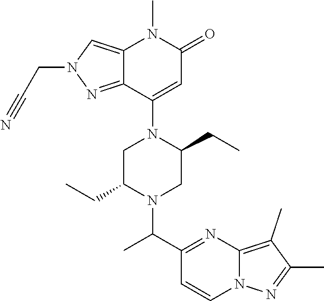 [SMILES: CC[C@H]1CN(C(C)c2ccn3nc(C)c(C)c3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (4 mL) and was added 1-(2,3-dimethylpyrazolo[1,5-a]pyrimidin-5-yl)ethan-1-ol (87 mg, 0.45 mmol), (cyanomethyl)trimethylphosphonium iodide (147 mg, 0.32 mmol) and DIPEA(197 mg, 1.5 mmol).The resulting mixture was stirred at 105° C. overnight. The reaction solvent was removed under reduce pressure. The resulting residue was purified by flash column chromatography to give the titled compound(crude), which was further separated into Compound A156a (1 mg, 9%) and Compound A156b (1.4 mg, 9%) by Prep-HPLC (Method A).

Compound A156a (the earlier peak): ¹H NMR (400 MHz, DMSO-d,) δ 8.85 (d, J=7.2 Hz, 1H), 7.98 (s, 1H), 6.98 (d, J=7.2 Hz, 1H), 5.61 (s, 2H), 5.39 (s, 1H), 5.20-4.00 (m, 2H), 3.67 (d, J=6.5 Hz, 1H), 3.26 (s, 3H), 3.03 (d, J=9.7 Hz, 1H), 2.66 (s, 2H), 2.33 (s, 3H), 2.19 (s, 1H), 2.17 (s, 3H), 1.91-0.39 (m, 4H), 1,29 (d, J=6.5 Hz, 3H), 0.93 (t, J=7.1 Hz, 3H), 0.58 (t, J=7.0 Hz, 3H) ppm. MS: M/e 502 (M+1)⁺.

Compound A156b (the later peak): ¹H NMR (400 MHz, DMSO-d₆) δ 8.82 (d, J=7.2 Hz, 1H), 7.97 (s, 1H), 6.95 (d, J=7.2 Hz, 1H), 5.61 (s, 2H), 5.39 (s, 1H), 4.41 (s, 2H), 3.86 (q, J=6.4 Hz, 1H), 3.26 (s, 3H), 3.19 (d, J=11.3 Hz, 1H), 2.85 (d, J=11.9 Hz, 2H), 2.38 (d, J=8.6 Hz, 1H), 2.34 (s, 3H), 2.17 (s, 3H), 1.99-1.46 (m, 4H), 1.33 (d, J=6.6 Hz, 3H), 0.81 (t, J=7.3 Hz, 3H), 0.67 (t, J=7.3 Hz, 3H) ppm. MS: We 502 (M+1)⁺.

Compound A157: 2-(7-((2S,5R)-2,5-diethyl-4-(1-(2-methylpyrazolo[1,5-a]pyrimidin-5-yl)ethyl)piperazin-1-yl)-4-methyl-5-oxo-4,5-dihydro-2H-pyrazolo[4,3-d]pyrimidin-2-yl)acetonitrile

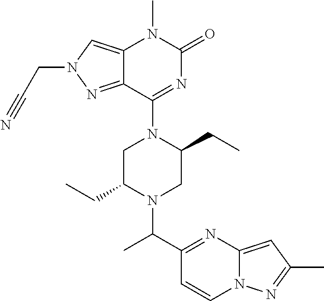 [SMILES: CC[C@H]1CN(C(C)c2ccn3nc(C)cc3n2)[C@H](CC)CN1c1nc(=O)n(C)c2cn(CC#N)nc12]

### Step A: tert-butyl (2S,5R)-2,5-diethyl-4-(1-(2-methylpyrazolo[1,5-a]pyrimidin-5-yl)ethyl)piperazine-1-carboxylate

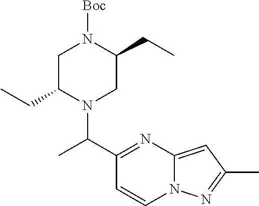 [SMILES: CC[C@H]1CN(C(C)c2ccn3nc(C)cc3n2)[C@H](CC)CN1C(=O)OC(C)(C)C]

To a solution of tert-butyl (2S,5R)-2,5-diethylpiperazine-1-carboxylate (242 mg, 1 mmol), 1-(2-methylpyrazolo[1,5-a]pyrimidin-5-yl)ethan-1-ol (352 mg, 2 mmol) and DIPEA (645 mg, 5 mmol) in CH₃CN (6 mL) was added (cyanomethyl)trimethylphosphonium iodide (730 mg, 3 mmol). The mixture was stirred at 100° C. in a sealed bottle for 16 hours. The reaction was cooled to room temperature, diluted by adding water, extracted with EtOAc (80 mL×2), washed with brine, dried over Na₂SO₄, filtered and concentrated to dryness. The residue was purified by flash column chromatography to give the titled compound (357 mg, 89%). MS: M/e 402 (M+1)⁺.

### Step B: 5-(1-((2R,5S)-2,5-diethylpiperazin-1-yl)ethyl)-2-methylpyrazolo[1,5-a]pyrimidine

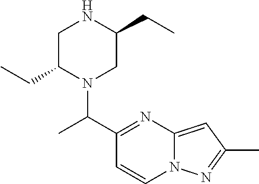 [SMILES: CC[C@H]1CN(C(C)c2ccn3nc(C)cc3n2)[C@H](CC)CN1]

To a solution of tert-butyl (2S,5R)-2,5-diethyl-4-(1-(2-methylpyrazolo[1,5-a]pyrimidin-5-yl)ethyl)piperazine-1-carboxylate (358 mg, 0.89 mmol) in DCM (10 mL) was added TMSOTf (395 mg, 1.78 mmol). The resulted mixture was stirred at room temperature for 16 hours. The reaction mixture was quenched with aq. NaHCO₃, then extracted with CH₂Cl₂/IPA (3/1, 60 mL x 4). The combined organic layers were dried over Na₂SO₄, concentrated to give the target compound (230 mg, 86%). MS: M/e 302 (M+1)⁺.

### Step C: 7-((2S,5R)-2,5-diethyl-4-(1-(2-methylpyrazolo[1,5-a]pyrimidin-5-yl)ethyl) piperazin-1-yl)-4-methyl-2-(tetrahydro-2H-pyran-2-yl)-2,4-dihydro-5H-pyrazolo[4,3-d]pyrimidin-5-one

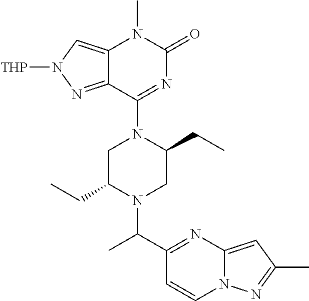

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5 mmol) in CH₃CN (3 mL) was added DIEA (129 mg, 1 mmol), followed by POCl₃ (153 mg, 1 mmol) and catalyst amount of DMF. The reaction was stirred at 80° C. for 1 h. The reaction was cooled to room temperature, quenched with ice-water, extracted with EtOAc (60 mL×2), washed with brine, dried over Na₂SO₄, filtered, and concentrated.

The above residue was dissolved in CH₃CN (5 mL). 5-(1-((2R,5S)-2,5-diethylpiperazin-1-yl)ethyl)-2-methylpyrazolo[1,5-a]pyrimidine (100 mg, 0.3 mmol) and DIEA (129 mg, 1 mmol) were added. The resulting mixture was heated at 85° C. for 16 hours. The reaction was quenched with water, extracted with EtOAc (60 mL×2), washed with brine, dried over Na₂SO₄, filtered, and concentrated. The residue was purified by flash column chromatography to give target compound (30 mg, 11% for two steps). MS: M/e 534 (M+1)⁺.

### Step D: 7-((2S,5R)-2,5-diethyl-4-(1-(2-methylpyrazolo[1,5-a]pyrimidin-5-yl)ethyl) piperazin-1-yl)-4-methyl-2,4-dihydro-5H-pyrazolo[4,3-d]pyrimidin-5-one

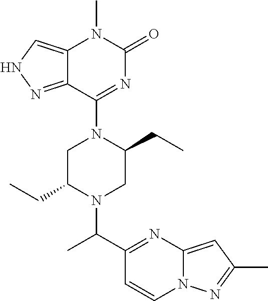 [SMILES: CC[C@H]1CN(C(C)c2ccn3nc(C)cc3n2)[C@H](CC)CN1c1nc(=O)n(C)c2c[nH]nc12]

To a solution of 7-((2S,5R)-2,5-diethyl-4-(1-(2-methylpyrazolo[1,5-a]pyrimidin-5-yl)ethyl)piperazin-1-yl)-4-methyl-2-(tetrahydro-2H-pyran-2-yl)-2,4-dihydro-5H-pyrazolo[4,3-d]pyrimidin-5-one (30 mg, 0.056 mmol) in DCM (1 mL) was added TFA (3 mL). The resulted mixture was stirred at room temperature for 16 hours. The mixture was concentrated under reduced pressure to give target compound (crude). MS: M/e 450 (M+1)⁺.

### Step E: 2-(7-(2S,5R)-2,5-diethyl-4-(1-(2-methylpyrazolo[1,5-a]pyrimidin-5-yl) ethyl)piperazin-1-yl)-4-methyl-5-oxo-4,5-dihydro-2H-pyrazolo[4,3-d]pyrimidin-2-yl)acetonitrile

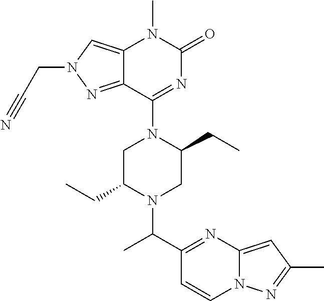 [SMILES: CC[C@H]1CN(C(C)c2ccn3nc(C)cc3n2)[C@H](CC)CN1c1nc(=O)n(C)c2cn(CC#N)nc12]

To a mixture of 7-((2S,5R)-2,5-diethyl-4-(1-(2-methylpyrazolo[1,5-a]pyrimidin-5-yl)ethyl)piperazin-1-yl)-4-methyl-2,4-dihydro-5H-pyrazolo[4,3-d]pyrimidin-5-one (30 mg, crude) and K₂CO₃ (103 mg, 0.75 mmol) in DMF (2 mL) was added 2-chloroacetonitrile (30 mg, 0.375 mmol). The reaction was stirred at room temperature for 16 hours. The reaction was diluted with water, extracted with EtOAc (50 mL×2), washed with brine, dried over Na₂SO₄, filtered, and concentrated. The resulting residue was purified and separated into Compound A157a (5 mg, 18.5% for two steps) and Compound A157b (7 mg, 25% for two steps) by Prep-HPLC (Method A).

Compound A157a (the earlier peak): ¹HNMR (400 MHz, CD₃OD) δ 8.75 (d, J=7.2 Hz, 1H), 7.92 (d, J=7.2 Hz, 1H), 7.24-7.18 (m, 1H), 6.41 (s, 1H), 5.84-5.76 (m, 0.5H), 5.57-5.45 (m, 2,5H), 5.28-5.21 (m, 0.5H), 5.04-4.98 (m, 0.5H), 4.64-4.59 (m, 0.5H), 3.82-3.62 (m, 1.5H), 3.41 (s, 3H), 3.26-3.10 (m, 1H), 2.87-2.68 (m, 1H), 2,48 (s, 3H), 2.34-2.26 (m, 1H), 2.10-1.75 (m, 2H), 1.58-1.45 (m, 2H), 1.38 (d, .=6.4 Hz, 3H), 1.10-0.95 (m, 3H), 0.74 (d, J=7.6 Hz, 3H) ppm. MS: M/e 489 (M+1)⁺.

Compound A157b (the later peak): ¹HNMR (400 MHz, CD₃OD) δ 8.74 (d, J=6.8 Hz, 1H), 7.92 (d, J=10.0 Hz, 1H), 7.20-7.15 (m, 1H), 6.42 (s, 1H), 5.79-5.70 (m, 0.5H), 5.63-5.45 (m, 2,5H), 5.26-5.16 (m, 0.5H), 5.06-4.02 (m, 0.5H), 3.98-3.86 (m, 1H), 3.63-3.54 (m, 0.5H), 3.40 (s, 3H), 3.20-3.12 (m, 05H), 3.05-2.80 (m, 2H), 2,55-2,43 (m, 4H), 2.17-1.93 (m, 2H), 1.66-1.35 (m, 5H), 0.99-0.89 (m, 3H), 0.83-0.70 (m, 3H) ppm. MS: M/e 489 (M+1)⁺.

### Compound A158: 2-(7-((2S,5R)-2,5-diethyl-4-(1-(2-fluoropyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile

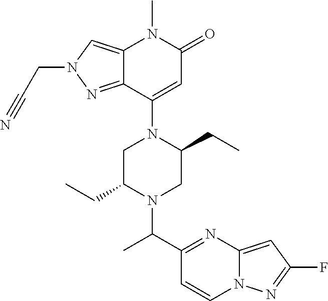 [SMILES: CC[C@H]1CN(C(C)c2ccn3nc(F)cc3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(2-fluoropyrazolo[1,5-a]pyrimidin-5-yl)ethan-1-ol (28 mg, 0.15 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduce pressure to afford crude product. The resulting residue was purified and separated into Compound A158a (20 mg, 53%) and Compound A158b (18 mg, 47%) by Prep-HPLC (Method A).

Compound A158a (the earlier peak): ¹H NMR (400 MHz, CD₃OD) δ 8.74 (d, J=7.3 Hz, 1H), 7.94 (s, 1H), 7.32 (d, J=7.2 Hz, 1H), 6.18 (d, J=5.0 Hz, 1H), 5.57 (s, 1H), 5.48 (s, 2H), 4.86-4.26 (m, 2H), 3.80-3.74 (m, 1H), 3.55 (d, J=13.0 Hz, 1H), 3.44 (s, 3H), 3.14 (d, .=9.1 Hz, 1H), 2.85-2.79 (m, 1H), 2.33 (d, J=12.1 Hz, 1H), 1.98-1.89 (m, 1H), 1.81-1.65 (m, 2H), 1.56-1.48 (m, 1H), 1.39 (d, J=6.7 Hz, 3H), 1.03 (t, J=7.4 Hz, 3H), 0.72 (t, J=7.5 Hz, 3H) ppm. MS: M/e 492 (M+1)⁺.Compound A158b (the later peak): ¹H NMR (400 MHz, CD₃OD) δ 8.73 (d, J=7.2 Hz, 1H), 7.93 (s, 1H), 7.27 (d, J=7.2 Hz, 1H), 6.19 (d, J=4.8 Hz, 1H), 5.58 (s, 1H), 5.47 (s, 2H), 4.85-4.48 (m, 2H), 3.96 (d, J=6.8 Hz, 1H), 3.44 (s, 3H), 3.39 (d, J=13.7 Hz, 1H), 2.97 (s, 2H), 2,48 (s, 1H), 2.13-2.04 (m, 1H), 1.88-1.80 (m, 1H), 1.66-1.58 (m, 2H), 1.41 (d, J=6.7 Hz, 3H), 0.94 (t, J=7.4 Hz, 3H), 0.79 (t, J=7.4 Hz, 3H) ppm. MS: M/e 492 (M+1)⁺.

Compound A159: 2-(7-((2S,5R)-2,5-diethyl-4-(1-(pyrazolo[1,5-aJ pyridin-5-yl)ethyl)piperazin-1-yl)-4-methyl-5-oxo-4,5-dihydro-2H-pyrazolo[4,3-d]pyrimidin-2-yl)acetonitrile

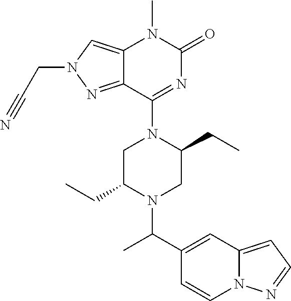 [SMILES: CC[C@H]1CN(C(C)c2ccn3nccc3c2)[C@H](CC)CN1c1nc(=O)n(C)c2cn(CC#N)nc12]

### Step A: tert-butyl (2S,5R)-2,5-diethyl-4-(1-(pyrazolo[1,5-a]pyridin-5-yl)ethyl) piperazine-1-carboxylate

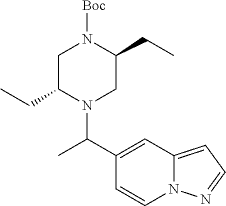 [SMILES: CC[C@H]1CN(C(C)c2ccn3nccc3c2)[C@H](CC)CN1C(=O)OC(C)(C)C]

To a solution of tert-butyl (2S,5R)-2,5-diethylpiperazine-1-carboxylate (242 mg, 1 mmol), 1-(pyrazolo[1,5-a]pyridin-5-yl)ethan-1-ol (243 mg, 1.5 mmol) and DIPEA (645 mg, 5 mmol) in CH₃CN (5 mL) was added (cyanomethyl)trimethylphosphonium iodide (730 mg, 3 mmol). The mixture was stirred at 103° C. in a sealed bottle for 16 hours. The reaction was cooled to room temperature, diluted with water, extracted with EtOAc (80 mL×2), washed with brine, dried over Na₂SO₄, filtered and concentrated to dryness. The residue was purified by flash column chromatography to give the titled compound (260 mg, crude). MS: M/e 387 (M+1)⁺.

### Step B: 5-(1-((2R,5S)-2,5-diethylpiperazin-1-yl)ethyl)pyrazolo[1,5-a]pyridine

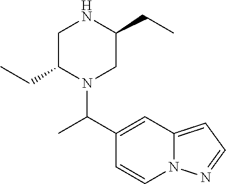 [SMILES: CC[C@H]1CN(C(C)c2ccn3nccc3c2)[C@H](CC)CN1]

To a solution of tert-butyl (2S,5R)-2,5-diethyl-4-(1-(pyrazolo[1,5-a]pyridin-5-yl)ethyl)piperazine-1-carboxylate (260 mg, 0.67 mmol) in DCM (5 mL) was added TFA (1 mL). The resulted mixture was stirred at room temperature for 16 hours. The reaction mixture was quenched with water, extracted with DCM (60 mL). The aqueous layer was collected, basified with aq. NaHCO₃, then extracted with CH₂Cl₂/IPA (3/1, 60 mL×4). The combined organic layers were dried over Na₂SO₄, concentrated to give the target compound (100 mg, 35% for two steps). MS: M/e 287 (M+1)⁺.

### Step C: 7-((2S,5R)-2,5-diethyl-4-(1-(pyrazolo[1,5-a]pyridin-5-yl)ethyl)piperazin-1-yl)-4-methyl-2-(tetrahydro-2H-pyran-2-yl)-2,4-dihydro-5H-pyrazolo[4,3-d]pyrimidin-5-one

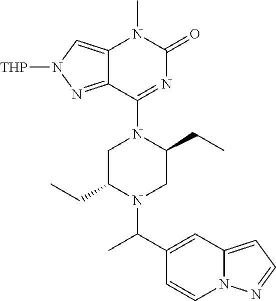

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5 mmol) in CH₃CN (3 mL) was added DIEA (129 mg, 1 mmol), followed by POCl₃ (153 mg, 1 mmol) and catalyst amount of DMF. The reaction was stirred at 80° C. for 1.5 hours. The reaction was cooled to room temperature, quenched with ice-water, extracted with EtOAc (60 mL×2), washed with brine, dried over Na₂SO₄, filtered, and concentrated. To a mixture of 5-(1-((2R,5S)-2,5-diethylpiperazin-1-yl)ethyl)pyrazolo[1,5-a]pyridine (100 mg, 0.34 mmol) in CH₃CN (5 mL) was added DIEA (129 mg, 1 mmol), follow by a solution of the above residue in CH₃CN (2 mL). The resulting mixture was heated at 90° C. for 3 hours. The reaction was quenched with water, extracted with EtOAc (70 mL×2), washed with brine, dried over Na₂SO₄, filtered, and concentrated. The residue was purified by flash column chromatography to give target compound (80 mg, 30% for two steps). MS: M/e 519 (M+1)⁺.

### Step D: 7-((2S,5R)-2,5-diethyl-4-(1-(pyrazolo[1.5-a]pyridin-5-yl)ethyl)piperazin-1-yl)-4-methyl-2,4-dihydro-5H-pyrazolo[4,3-d]pyrimidin-5-one

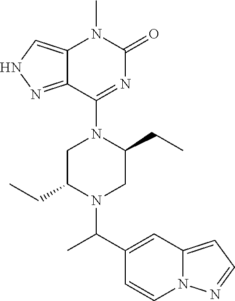 [SMILES: CC[C@H]1CN(C(C)c2ccn3nccc3c2)[C@H](CC)CN1c1nc(=O)n(C)c2c[nH]nc12]

To a solution of 7-((2S,5R)-2,5-diethyl-4-(1-(pyrazolo[1,5-a]pyridin-5-yl)ethyl)piperazin-1-yl)-4-methyl-2-(tetrahydro-2H-pyran-2-yl)-2,4-dihydro-5H-pyrazolo[4,3-d]pyrimidin-5-one (80 mg, 0.15 mmol) in DCM (1 mL) was added TFA (3 mL). The resulted mixture was stirred at room temperature for 16 hours. The mixture was concentrated under reduced pressure to give titled compound (80 mg, crude). MS: M/e 435 (M+1)⁺.

### Step E: 2-(7-((2S,5R)-2,5-diethyl-4-(1-(pyrazolo[1,5-a]pyridin-5-yl)ethyl) piperazin-1-yl)-4-methyl-5-oxo-4,5-dihydro-2H-pyrazolo[4,3-d]pyrimidin-2-yl)acetonitrile

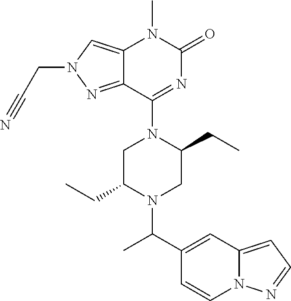 [SMILES: CC[C@H]1CN(C(C)c2ccn3nccc3c2)[C@H](CC)CN1c1nc(=O)n(C)c2cn(CC#N)nc12]

To a mixture of 7-((2S,5R)-2,5-diethyl-4-(1-(pyrazolo[1,5-a]pyridin-5-yl)ethyl)piperazin-1-yl)-4-methyl-2,4-dihydro-5H-pyrazolo[4,3-d]pyrimidin-5-one (80 mg, crude) and K₂CO₃ (210 mg, 1.5 mmol) in DMF (2 mL) was added 2-chloroacetonitrile (56 mg, 0.75 mmol). The reaction was stirred at room temperature for 16 hours. The reaction was diluted with water, extracted with EtOAc (70 mL), washed with brine, dried over Na₂SO₄, filtered, and concentrated. The resulting residue was purified and separated into Compound A159a (5 mg, 18.3% for two steps) and Compound A159b (7 mg, 10% for two steps) by Prep-HPLC (Method A).

Compound A159a (the earlier peak): ¹HNMR (400 MHz, CD₃OD) δ 8.51 (d, J=7.2 Hz, 1H), 7.95-7.88 (m, 2H), 7.57 (s, 1H), 7.04 (d, J=5.6 Hz, 1H), 6.55 (d, J=2.0 Hz, 1H), 5.78-5.68 (m, 0.5H), 5.61-5.45 (m, 2,5H), 5.26-5.16 (m, 0.5H), 5.04-4.96 (m, 0.5H), 3.87-3.75 (m, 1H), 3.61-3.52 (m, 0.5H), 3.41 (s, 1.5H), 3.40 (s, 1.5H), 3.18-3.10 (m, 0.5H), 3.08-3.00 (m, 1H), 3.94-2.86 (m, 0.5H), 2.84-2.76 (m, 0.5H), 2.61-2,46 (m, 1H), 2.26-1.95 (m, 2H), 1.65-1.49 (m, 1H), 1.44-1.32 (m, 4H), 1.00-0.90 (m, 3H), 0.77 (t, J=8.0 Hz, 1.5H), 0.70 (t, J=8.0 Hz, 1.5H) ppm. MS: M/e 474 (M+1)⁺.

Compound A159b (the later peak): ¹HNMR (400 MHz, CD₃OD) δ 8.48 (d, J=6.8 Hz, 1H), 7.96-7.88 (m, 2H), 7.60 (s, 1H), 7.05 (d, J=7.6 Hz, 1H), 6.53 (d, J=1,2 Hz, 1H), 5.85-5.76 (m, 0.5H), 5.56-5.44 (m, 2,5H), 5.28-5.19 (m, 0.5H), 5.06-4.96 (m, 0.5H), 3.80-3.72 (m, 0.5H), 3.70-3.60 (m, 1H), 3.41 (s, 1.5H), 3.40 (s, 1.5H), 3.37-3.32 (m, 0.5H), 3.26-3.10 (m, 1H), 2.76-2.60 (m, 1H), 2,48-2.38 (m, 1H), 2.05-1.75 (m, 2H), 1.60-1.44 (m, 2H), 1.36 (d, J=6.8 Hz, 3H), 1.10-0.96 (m, 3H), 0.71 (t, J=7.6 Hz, 3H) ppm. MS: M/e 474 (M+1)⁺.

### Compound A160: 2-(7-((2S,5R)-2,5-diethyl-4-(1-(3-fluoro-2-methylpyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile

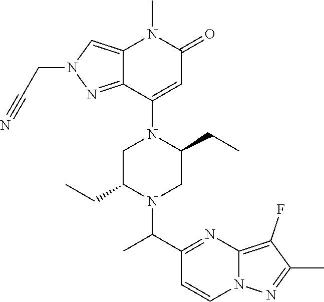 [SMILES: CC[C@H]1CN(C(C)c2ccn3nc(C)c(F)c3n2)[C@H](CC)CN1c1cc(=O)n(C)c2cn(CC#N)nc12]

### Step A: 5-chloro-2-methylpyrazolo[1,5-a]pyrimidine

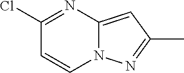

To a solution of 2-methylpyrazolo[1,5-a]pyrimidin-5 (4H)-one (0.5 g, 3.36 mmol) in CH₃CN (6 mL) were added POCl₃ (1 g, 6.7 mmol). The mixture was stirred at 100° C. for 3 hours. The reaction was concentrated and poured into saturated NaHCO₃ aq., extracted with DCM. The combined organic layers were washed with brine, dried over Na₂SO₄ and concentrated to give the titled compound (0.33 g, 56%). MS: M/e 168 (M+1)⁺.

### Step B: 1-(2-methylpyrazolo[1,5-a]pyrimidin-5-yl)ethan-1-one

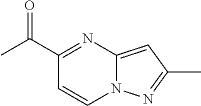

A mixture of 5-chloro-2-methylpyrazolo[1,5-a]pyrimidine (0.33 g, 1.9 mmol), tributyl(1-ethoxyvinyl)stannane (0.86 g, 2,4 mmol) and Pd(PPh₃)₂Cl₂ (139 mg, 0.19 mmol) in DMF (5 mL) was stirred at 100° C. under N₂ for 16 hours. The solution was diluted with ethyl acetate and washed with water. The organic layer was purified by flash column chromatography to give the 5-(1-ethoxyvinyl)-2-methylpyrazolo [1,5-a]pyrimidine, then diluted with DCM and added with HCl (5 mL, 4 M in dioxane) in drops and the mixture was stirred at RT for 2 hours. The reaction was concentrated and adjusted to pH=8-9 with saturated NaHCO₃ solution, extracted with ethyl acetate. The combined organic layers were washed with brine, dried over Na₂SO₄ and concentrated to dryness. The resulting residue was purified by flash column chromatography to give the titled compound (0.22 g, 76%). MS: M/e 176 (M+1)⁺

### Step C: 1-(3-fluoro-2-methylpyrazolo[1,5-a]pyrimidin-5-yl)ethan-1-one

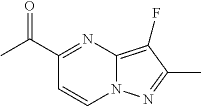

To a solution of 1-(2-methylpyrazolo[1,5-a]pyrimidin-5-yl)ethan-1-one (0.7 g, 4 mmol) in CH₃CN (25 mL) were added 1-Chloromethyl-4-fluoro-1,4-diazoniabicyclo[2.2.2]octane bis(tetrafluoroborate) (3.5 g, 10 mmol) was stirred at room temperature under N₂ for 30 mins. The solution was purified by flash column chromatography to give the titled compound (0.29 g, 37%). MS: M/e 194 (M+1)⁺

### Step D: 1-(3-fluoro-2-methylpyrazolo[1,5-a]pyrimidin-5-yl)ethan-1-ol

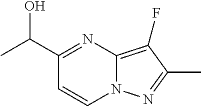

To a solution of 1-(3-fluoro-2-methylpyrazolo[1,5-a]pyrimidin-5-yl)ethan-1-one (0.29 g, 1.5 mmol) in MeOH (5 mL) was added NaBH₄ (34 mg, 0.9 mmol) at room temperature and the resulting mixture was stirred at room temperature for 5 mins. The reaction mixture was diluted with DCM and washed with water, dried over Na₂SO₄, concentrated to give the titled compound (270 mg). MS: M/e 196 (M+1)⁺.

### Step E: 2-(7-((2S,5R)-2,5-diethyl-4-(1-(3-fluoro-2-methylpyrazolo[1.5-al pyrimidin-5-yl)ethyl)piperazin-1-yl)-4-methyl-5-oxo-4,5-dihydro-2H-pyrazolo[4,3-b]pyridin-2-yl)acetonitrile

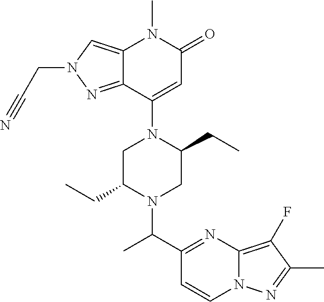 [SMILES: CC[C@H]1CN(C(C)c2ccn3nc(C)c(F)c3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (5 mL) and was added 1-(2-fluoro-3-methylpyrazolo[1,5-a]pyrimidin-5-yl)ethan-1-ol (87 mg, 0.45 mmol), (cyanomethyl)trimethylphosphonium iodide (146 mg, 0.6 mmol) and DIPEA(197 mg, 1.5 mmol).The resulting mixture was stirred at 105° C. overnight. The reaction solvent was removed under reduce pressure. The resulting residue was purified by flash column chromatography to give the titled compound(crude), which was further separated into Compound A160a (5 mg, 6%) and Compound A160b (7 mg, 9%) by Prep-HPLC (Method A).

Compound A160a (the earlier peak): ¹H NMR (400 MHz, DMSO-d⁶) δ 8.89 (d, J=7.3 Hz, 1H), 7.99 (s, 1H), 7.09 (d, J=7.3 Hz, 1H), 5.62 (s, 2H), 5.40 (s, 1H), 5.19-3.89 (m, 2H), 3.70 (d, J=6.8 Hz, 1H), 3.40 (d, J=13.2 Hz, 1H), 3.27 (s, 3H), 3.03 (d, J=9.6 Hz, 1H), 2.68 (d, J=8.4 Hz, 1H), 2,40 (s, 3H), 2.22 (d, 0.1=11.2 Hz, 1H), 1.87-1.37 (m, 4H), 1.31 (d, J=6.6 Hz, 3H), 0.93 (t, J=7.3 Hz, 3H), 0.60 (t, J=7.3 Hz, 3H) ppm. MS: M/e 506 (M+1)⁺.

Compound A160b (the later peak): ¹H NMR (400 MHz, DMSO-d₆) δ 8.86 (d, J=7.2 Hz, 1H), 7.98 (s, 1H), 7.07 (d, J=7.3 Hz, 1H), 5.61 (s, 2H), 5.40 (s, 1H), 5.16-3.99 (m, 2H), 3.89 (t, J=6.5 Hz, 1H), 3.27 (s, 3H), 3.22 (d, J=12.3 Hz, 1H), 2.84 (s, 2H), 2,43 (s, 1H), 2,41 (s, 3H), 1.98-1.47 (m, 4H), 1.34 (d, J=6.6 Hz, 3H), 0.81 (t, J=7.4 Hz, 3H), 0.71 (t, J=7.3 Hz, 3H) ppm. MS: M/e 506 (M+1)⁺.

### Compound A161: 2-(7-((2S,5R)-2,5-diethyl-4-(1-(3-fluoropyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile

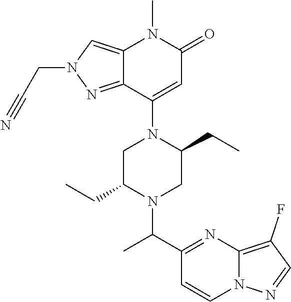 [SMILES: CC[C@H]1CN(C(C)c2ccn3ncc(F)c3n2)[C@H](CC)CN1c1cc(=O)n(C)c2cn(CC#N)nc12]

### Step A: 3-fluoropyrazolo[1.5-a]pyrimidin-5 (4H)-one

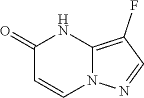 [SMILES: O=c1ccn2ncc(F)c2[nH]1]

A mixture of 4-fluoro-1H-pyrazol-5-amine (0.5 g, 5.1 mmol), ethyl (E)-3-ethoxyacrylate (1.4 g, 10 mmol) and Cs₂CO₃ (3.2 g, 10 mmol) in DMF (20 mL) was stirred at 110° C. under N₂ for 16 hours. The solution was collected by filtration, then added acetic acid (5 mL). The organic layer was concentrated to dryness. The resulting residue was purified by flash column chromatography to give the titled compound (0.7 g, 93%). MS: M/e 154 (M+1)⁺.

### Step B: 5-chloro-3-fluoropyrazolo[1.5-a]pyrimidine

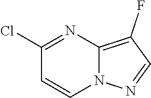 [SMILES: Fc1cnn2ccc(Cl)nc12]

To a solution of 3-fluoropyrazolo[1,5-a]pyrimidin-5 (4H)-one (0.7 g, 4.5 mmol) in CH₃CN (10 mL) were added POCl₃ (1.4 g, 9.1 mmol). The mixture was stirred at 90° C. for 3 hours. The reaction was concentrated and poured into saturated NaHCO₃ solution, extracted with DCM. The combined organic layers were washed with brine, dried over Na₂SO₄ and concentrated to dryness. The resulting residue was purified by flash column chromatography to give the titled compound (0.3 g, 38%). MS: M/e 172 (M+1)⁺.

### Step C: 1-(3-fluoropyrazolo[1.5-a]pyrimidin-5-ylethan-1-one

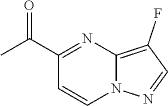

A mixture of 5-chloro-3-fluoropyrazolo[1,5-a]pyrimidine (0.3 g, 1.75 mmol), tributyl(1-ethoxyvinyl)stannane (0.76 g, 2.1 mmol) and Pd(PPh₃)₂Cl₂ (123 mg, 0.17 mmol) in DMF (5 mL) was stirred at 100° C. under N₂ for 16 hours. The solution was diluted with EA and washed with water. The organic layer was concentrated to dryness. The resulting residue was purified by flash column chromatography to give the titled compound (0.21 g, 66%). MS: M/e 180 (M+1)⁺

### Step D: 1-(3-fluoropyrazolo[1,5-a]pyrimidin-5-yl)ethan-1-ol

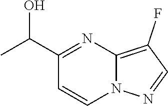

To a solution of 1-(3-fluoropyrazolo[1,5-a]pyrimidin-5-yl)ethan-1-one (0.21 g, 1.17 mmol) in MeOH (5 mL) was added NaBH₄ (27 mg, 0.7 mmol) at room temperature and the resulting mixture was stirred at room temperature for 5 mins. The reaction mixture was diluted with DCM and washed with water, dried over Na₂SO₄ and concentrated to give the titled compound (150 mg). MS: M/e 182 (M+1)⁺.

### Step E: 2-(7-((2S,5R)-2,5-diethyl-4-(1-(3-fluoropyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile

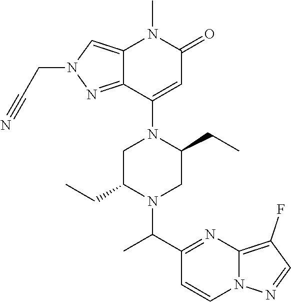 [SMILES: CC[C@H]1CN(C(C)c2ccn3ncc(F)c3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro -2H-pyrazolo[4,3-b]pyridin-2-yl)acetonitrile (50 mg, 0.15 mmol) in CH₃CN (5 mL) and was added 1-(3-fluoropyrazolo[1,5-a]pyrimidin-5-yl)ethan-1-ol (81 mg, 0.52 mmol), (cyanomethyl)trimethylphosphonium iodide (148 mg, 0.6 mmol) and DIPEA(196 mg, 1.5 mmol).The resulting mixture was stirred at 105° C. overnight. The reaction solvent was removed under reduce pressure. The resulting residue was purified by flash column chromatography to give the titled compound(crude), which was further separated into Compound A161a (11 mg, 14%) and Compound A161b (6 mg, 8%) by Prep-HPLC (Method A).

Compound A161a (the earlier peak): ¹H NMR (400 MHz, DMSO-d₆) δ 8.99 (d, J=7.3 Hz, 1H), 8.31 (d, J=3.3 Hz, 1H), 7.98 (s, 1H), 7.18 (d, J=7.4 Hz, 1H), 5.61 (s, 2H), 5.40 (s, 1H), 5.01-3.99 (m, 2H), 3.73 (d, J=6.6 Hz, 1H), 3.41 (d, J=13.3 Hz, 1H), 3.27 (s, 3H), 3.03 (d, J=9.6 Hz, 1H), 2.68 (d, J=13.1 Hz, 1H), 2.22 (d, J=12.0 Hz, 1H), 1.89-1.39 (m, 4H), 1.31 (d, J=6.5 Hz, 3H), 0.93 (t, J=7.3 Hz, 3H), 0.60 (t, J=7.2 Hz, 3H) ppm. MS: M/e 492 (M+1)⁺.

Compound A161b (the later peak): ¹H NMR (400 MHz, DMSO-d₆) δ 8.96 (d, J=7.3 Hz, 1H), 8.31 (d, J=3.3 Hz, 1H), 7.98 (s, 1H), 7.16 (d, J=7.4 Hz, 1H), 5.61 (s, 2H), 5.40 (s, 1H), 5.00-4.10 (m, 2H), 3.93 (q, J=6.4 Hz, 1H), 3.27 (s, 3H), 3.21 (s, 1H), 2.83 (d, J=12.7 Hz, 2H), 2,45 (s, 1H), 2.01-1.49 (m, 4H), 1.35 (d, J=6.6 Hz, 3H), 0.80 (t, J=7.3 Hz, 3H), 0.71 (t, J=7.2 Hz, 3H) ppm. MS: M/e 492 (M+1)⁺.

### Compound A162: 2-(7-((2S,5R)-2,5-diethyl-4-(1-(2-methylpyrazolo[1,5-a]pyridin-5-yl)ethyl)piperazin-1-yl)-4-methyl-5-oxo-4,5-dihydro-2H-pyrazolo [4,3-b]pyridin-2-yl)acetonitrile

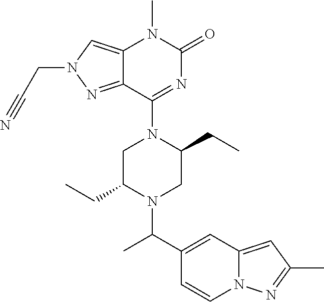 [SMILES: CC[C@H]1CN(C(C)c2ccn3nc(C)cc3c2)[C@H](CC)CN1c1nc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), 1-(2-methylpyrazolo[1,5-a]pyridin-5-yl)ethan-1-ol (35 mg, 0.2 mmol), (cyanomethyl)trimethylphosphonium iodide (85 mg, 0.35 mmol) and DIEA (65 mg, 0.5 mmol) in MeCN (1 mL) was stirred at 100° C. in a sealed tube for 16 hours. The mixture was diluted with EA (10 mL), washed with brine (5 mL×3), dried over Na₂SO₄, filtered and concentrated to dryness. The resulted residue was purified by column chromatography and further separated into Compound A162a (1.1 mg, 2.3%) and Compound A162b (1.6 mg, 3.4%) by Prep-HPLC (Method A).

Compound A162a (the earlier peak): ¹H NMR (400 MHz, CD₃OD) δ 8.37 (d, J=7.2 Hz, 1H), 7.92 (s, 1H), 7.43 (s, 1H), 6.93 (d, 0.1=7.2 Hz, 1H), 6.32 (s, 1H), 5.57 (s, 1H), 5.46 (s, 2H), 5.14-4.87 (m, 1H), 4.55-3.99 (m, 1H), 3.80 (q, J=6.4 Hz, 1H), 3.43 (s, 3H), 3.35 (s, 1H), 3.01 (d, J=11.6 Hz, 1H), 2.88 (dd, J=12.0, 3.6 Hz, 1H), 2,50 (s, 1H), 2,44 (s, 3H), 2.23-2.03 (m, 1H), 1.93-1.75 (m, 1H), 1.66-1.49 (m, 2H), 1.37 (d, J=6.4 Hz, 3H), 0.96 (t, J=7.2 Hz, 3H), 0.73 (t, J=7.2 Hz, 3H) ppm. MS: M/e 487 (M+1)⁺.

Compound A162b (the later peak): ¹H NMR (400 MHz, CD₃OD) δ 8.34 (d, J=7.2 Hz, 1H), 7.93 (s, 1H), 7.47 (s, 1H), 6.95 (d, J=7.2 Hz, 1H), 6.30 (s, 1H), 5.56 (s, 1H), 5.47 (s, 2H), 5.23-4.91 (m, 1H), 4.56-3.92 (m, 1H), 3.65 (q, J=6.4 Hz, 1H), 3.52 (d, J=11.6 Hz, 1H), 3.44 (s, 3H), 3.14 (d, J=10.0 Hz, 1H), 2.70 (dd, J=12.0, 3.6 Hz, 1H), 2,48-2.38 (m, 4H), 2.08-1.91 (m, 1H), 1.83-1.62 (m, 2H), 1.62-1.50 (m, 1H), 1.35 (d, J=6.8 Hz, 3H), 1.03 (t, J=7.2 Hz, 3H), 0.66 (t, J=7.2 Hz, 3H) ppm. MS: M/e 487 (M+1)⁺.

### Compound A163: 2-(7-((2S,5R)-2,5-diethyl-4-(1-(pyrazolo[1,5-a]pyrimidin-5-yl)ethyl)piperazin-1-yl)-4-methyl-5-oxo-4,5-dihydro-2H-pyrazolo[4,3-d]pyrimidin-2-yl)acetonitrile

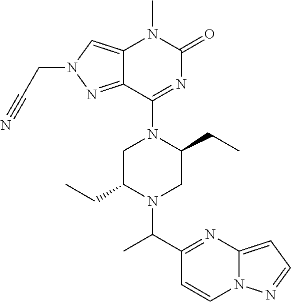 [SMILES: CC[C@H]1CN(C(C)c2ccn3nccc3n2)[C@H](CC)CN1c1nc(=O)n(C)c2cn(CC#N)nc12]

### Step A: tert-butyl (2S,5R)-2,5-diethyl-4-(1-(pyrazolo[1,5-a]pyrimidin-5-yl)ethyl) piperazine-1-carboxylate

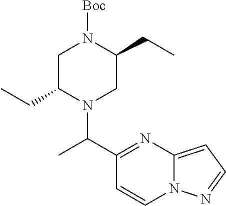 [SMILES: CC[C@H]1CN(C(C)c2ccn3nccc3n2)[C@H](CC)CN1C(=O)OC(C)(C)C]

To a solution of tert-butyl (2S,5R)-2,5-diethylpiperazine-1-carboxylate (242 mg, 1 mmol), 1-(pyrazolo[1,5-a]pyrimidin-5-yl)ethan-1-ol (326 mg, 2 mmol) and DIPEA (645 mg, 5 mmol) in CH₃CN (6 mL) was added (cyanomethyl)trimethylphosphonium iodide (730 mg, 3 mmol). The mixture was stirred at 103° C. in a sealed bottle for 16 hours. The reaction was cooled to room temperature, diluted with water, extracted with EtOAc (70 mL×2), washed with brine, dried over Na₂SO₄, filtered and concentrated to dryness. The residue was purified by flash column chromatography to give the titled compound (205 mg, 52%). MS: We 388 (M+1)⁺.

### Step B: 5-(1-((2R,5S)-2,5-diethylpiperazin-1-yl)ethyl)pyrazolo[1.5-a]pyrimidine

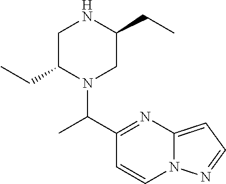 [SMILES: CC[C@H]1CN(C(C)c2ccn3nccc3n2)[C@H](CC)CN1]

To a solution of tert-butyl (2S,5R)-2,5-diethyl-4-(1-(pyrazolo[1,5-a]pyrimidin-5-yl)ethyl)piperazine-1-carboxylate (205 mg, 0.52 mmol) in DCM (5 mL) was added TFA (1 mL). The resulted mixture was stirred at room temperature for 16 hours. The reaction mixture was quenched with water, extracted with DCM (60 mL). The aqueous layer was collected, basified with aq. NaHCO₃, then extracted with CH₂Cl₂/IPA (3/1, 60 mL×4). The combined organic layers were dried over Na₂SO₄, concentrated to give the target compound (120 mg, 78%). MS: M/e 288 (M+1)⁺.

### Step C: 7-((2S,5R)-2,5-diethyl-4-(1-(pyrazolo[1,5-a]pyrimidin-5-yl)ethyl) piperazin-1-yl)-4-methyl-2-(tetrahydro-2H-pyran-2-yl)-2,4-dihydro-5H-pyrazolo[4,3-d]pyrimidin-5-one

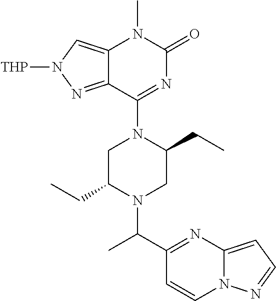

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5 mmol) in CH₃CN (3 mL) was added DIEA (129 mg, 1 mmol), followed by POCl₃ (153 mg, 1 mmol) and catalyst amount of DMF. The reaction was stirred at 80° C. for 1.5 hours. The reaction was cooled to room temperature, quenched with ice-water, extracted with EtOAc (60 mL×2), washed with brine, dried over Na₂SO₄, filtered, and concentrated. To a mixture of 5-(1-((2R,5S)-2,5-diethylpiperazin-1-yl)ethyl)pyrazolo[1,5-a]pyrimidine (120 mg, 0.4 mmol) in CH₃CN (5 mL) was added DIEA (129 mg, 1 mmol), follow by a solution of the above residue in CH₃CN (2 mL). The resulting mixture was heated at 90° C. for 4 hours. The reaction was quenched with water, extracted with EtOAc (60 mL×2), washed with brine, dried over Na₂SO₄, filtered, and concentrated. The residue was purified by flash column chromatography to give target compound (80 mg, 30% for two steps). MS: M/e 520 (M+1)⁺.

### Step D: 7-((2S,5R)-2,5-diethyl-4-(1-(pyrazolo[1,5-a]pyrimidin-5-yl)ethyl) piperazin-1-yl)-4-methyl-2,4-dihydro-5H-pyrazolo[4,3-d]pyrimidin-5-one

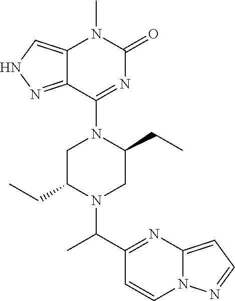 [SMILES: CC[C@H]1CN(C(C)c2ccn3nccc3n2)[C@H](CC)CN1c1nc(=O)n(C)c2c[nH]nc12]

To a solution of 7-((2S,5R)-2,5-diethyl-4-(1-(pyrazolo[1,5-a]pyrimidin-5-yl)ethyl)piperazin-1-yl)-4-methyl-2-(tetrahydro-2H-pyran-2-yl)-2,4-dihydro-5H-pyrazolo[4,3-d]pyrimidin-5-one (80 mg, 0.154 mmol) in DCM (1 mL) was added TFA (3 mL). The resulted mixture was stirred at room temperature for 16 hours. The mixture was concentrated under reduced pressure to give target compound (crude). MS: M/e 436 (M+1)⁺.

### Step E: 2-(7-((2S,5R)-2,5-diethyl-4-(1-(pyrazolo[1,5-a]pyrimidin-5-yl)ethyl) piperazin-1-yl) 4-methyl-5-oxo-4,5-dihydro-2H-pyrazolo[4,3-d]pyrimidin-2-yl)acetonitrile

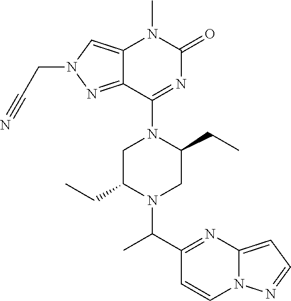 [SMILES: CC[C@H]1CN(C(C)c2ccn3nccc3n2)[C@H](CC)CN1c1nc(=O)n(C)c2cn(CC#N)nc12]

To a mixture of 7-((2S,5R)-2,5-diethyl-4-(1-(pyrazolo[1,5-a]pyrimidin-5-yl)ethyl)piperazin-1-yl)-4-methyl-2,4-dihydro-5H-pyrazolo[4,3-d]pyrimidin-5-one (80 mg, crude) and K₂CO₃ (212 mg, 1.54 mmol) in DMF (2 mL) was added 2-chloroacetonitrile (35 mg, 0.46 mmol). The reaction was stirred at room temperature for 16 hours. The reaction was diluted with water, extracted with EtOAc (50 mL×2), washed with brine, dried over Na₂SO₄, filtered, and concentrated. The resulting residue was purified and separated into Compound A163a (13 mg, 17.8% for two steps) and Compound A163b (10 mg, 13.6% for two steps) by Prep-HPLC (Method A).

Compound A163a (the earlier peak): ¹HNMR (400 MHz, CD₃OD) δ 8.88 (d, J=7.6 Hz, 1H), 8.13 (d, J=2.0 Hz, 1H), 7.92 (d, J=8.0 Hz, 1H), 7.34-7.27 (m, 1H), 6.61 (d, J=2.0 Hz, 1H), 5.86-5.75 (m, 0.5H), 5.60-5.45 (m, 2,5H), 5.29-5.20 (m, 0.5H), 5.08-4.98 (m, 0.5H), 3.83-3.72 (m, 1.5H), 3.41 (s, 3H), 3.39-3.34 (m, 0.5H), 3.26-3.12 (m, 1H), 2.87-2.71 (m, 1H), 2.35-2.27 (m, 1H), 2.11-1.74 (m, 2H), 1.59-1.46 (m, 2H), 1.40 (d, J=6.8 Hz, 3H), 1.10-0.96 (m, 3H), 0.75 (t, J=7.6 Hz, 3H) ppm. MS: M/e 475 (M+1)⁺.

Compound A163b (the later peak): ¹HNMR (400 MHz, CD₃OD) δ 8.87 (d, J=7.6 Hz, 1H), 8.13 (d, J=2.0 Hz, 1H), 7.91 (d, J=10.0 Hz, 1H), 7.32-7.23 (m,1H), 6.62 (d, J=2.0 Hz, 1H), 5.78-5.68 (m, 0.5H), 5.61-5.45 (m, 2,5H), 5.26-5.15 (m, 0.5H), 5.07-4.99 (m, 0.5H), 4.03-3.92 (m, 1H), 3.66-3.56 (m, 0.5H), 3.41 (s, 1.5H), 3.40 (s, 1.5H), 3.22-3.12 (m, 0.5H), 3.06-2.86 (m, 2H), 2,58-2,44 (m, 1H), 2.25-1.90 (m, 2H), 1.71-1.55 (m, 1H), 1.51-1.38 (m, 4H), 1.00-0.88 (m, 3H), 0.81 (t, J=7.6 Hz, 1.5H), 0.74 (t, J=7.6 Hz, 1.5H) ppm. MS: M/e 475 (M+1)⁺.

### Compound A164: 2-(7-((2S,5R)-2,5-diethyl-4-(1-(3-fluoropyrazolo[1,5-a]pyrimidin-5-yl)ethyl)piperazin-1-yl)-4-methyl-5-oxo-4,5-dihydro-2H-pyrazolo[4,3-d]pyrimidin-2-yl)acetonitrile

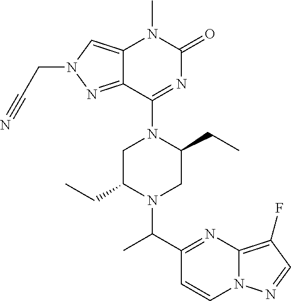 [SMILES: CC[C@H]1CN(C(C)c2ccn3ncc(F)c3n2)[C@H](CC)CN1c1nc(=O)n(C)c2cn(CC#N)nc12]

### Step A: tert-butyl (2S,5R)-2,5-diethyl-4-(1-(3-fluoropyrazolo[1,5-a]pyrimidin-5-yl)ethyl)piperazine-1-carboxylate

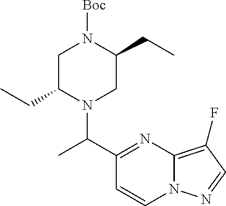 [SMILES: CC[C@H]1CN(C(C)c2ccn3ncc(F)c3n2)[C@H](CC)CN1C(=O)OC(C)(C)C]

To a solution of tert-butyl (2S,5R)-2,5-diethylpiperazine-1-carboxylate (242 mg, 1 mmol), 1-(3-fluoropyrazolo[1,5-a]pyrimidin-5-yl)ethan-1-ol (180 mg, 1 mmol) and DIPEA (645 mg, 5 mmol) in CH₃CN (6 mL) was added (cyanomethyl)trimethylphosphonium iodide (730 mg, 3 mmol). The mixture was stirred at 103° C. in a sealed bottle for 16 hours. The reaction was cooled to room temperature, diluted by adding water, extracted with EtOAc (70 mL×2), washed with brine, dried over Na₂SO₄, filtered and concentrated to dryness. The residue was purified by flash column chromatography to give the titled compound (300 mg, crude). MS: M/e 406 (M+1)⁺.

### Step B: 5-(1-((2R,5S)-2,5-diethylpiperazin-1-yl)ethyl)-3-fluoropyrazolo[1,5-a]pyrimidine

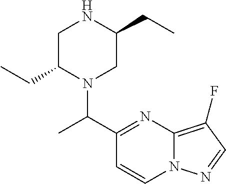 [SMILES: CC[C@H]1CN(C(C)c2ccn3ncc(F)c3n2)[C@H](CC)CN1]

To a solution of tert-butyl (2S,5R)-2,5-diethyl-4-(1-(3-fluoropyrazolo[1,5-a]pyrimidin-5-yl)ethyl)piperazine-1-carboxylate (300 mg, crude) in DCM (6 mL) was added TFA (1 mL). The resulted mixture was stirred at room temperature for 16 hours. The reaction mixture was quenched with water (50 mL), extracted with DCM (60 mL). The aqueous layer was collected, basified with aq. NaHCO₃, then extracted with CH₂Cl₂/MeOH (10/1, 40 mL×4). The combined organic layers were dried over Na₂SO₄, concentrated to give the target compound (120 mg, ³⁹⁰/ for two steps). MS: M/e 306 (M+1)⁺.

### Step C: 7-((2S,5R)-2,5-diethyl-4-(1-(3-fluoropyrazolo[1,5-a]pyrimidin-5-yl)ethyl) piperazin-1-yl)-4-methyl-2-(tetrahydro-2H-pyran-2-yl)-2,4-dihydro-5H-pyrazolo[4,3-d]pyrimidin-5-one

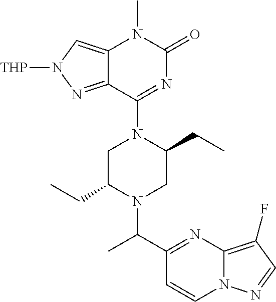

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5 mmol) in CH₃CN (3 mL) was added DIPEA (129 mg, 1 mmol), followed by POCl₃ (153 mg, 1 mmol) and catalyst amount of DMF. The reaction was stirred at 80° C. for 2 hours. The reaction was cooled to room temperature, quenched with ice-water, extracted with EtOAc (60 mL×2), washed with brine, dried over Na₂SO₄, filtered, and concentrated. To a mixture of 5-(1-((2R,5S)-2,5-diethylpiperazin-1-yl)ethyl)-3-fluoropyrazolo[1,5-a]pyrimidine (120 mg, 0.39 mmol) in CH₃CN (5 mL) was added DIEA (129 mg, 1 mmol), follow by a solution of the above residue in CH₃CN (2 mL). The resulting mixture was heated at 90° C. for 3 hours. The reaction was quenched with water, extracted with EtOAc (80 mL), washed with brine, dried over Na₂SO₄, filtered, and concentrated. The residue was purified by flash column chromatography to give target compound (72 mg, 26% for two steps). MS: M/e 538 (M+1)⁺.

### Step D: 7-((2S,5R)-2,5-diethyl-4-(1-(3-fluoropyrazolo[1.5-a]pyrimidin-5-yl)ethyl) piperazin-1-yl)-4-methyl-2,4-dihydro-5H-pyrazolo[4,3-d]pyrimidin-5-one

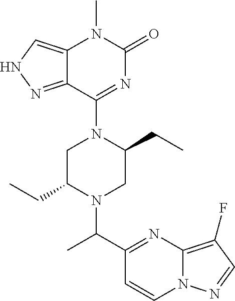 [SMILES: CC[C@H]1CN(C(C)c2ccn3ncc(F)c3n2)[C@H](CC)CN1c1nc(=O)n(C)c2c[nH]nc12]

To a solution of 7-((2S,5R)-2,5-diethyl-4-(1-(3-fluoropyrazolo[1,5-a]pyrimidin-5-yl)ethyl)piperazin-1-yl)-4-methyl-2-(tetrahydro-2H-pyran-2-yl)-2,4-dihydro-5H-pyrazolo[4,3-d]pyrimidin-5-one (72 mg, 0.13 mmol) in DCM (1 mL) was added TFA (3 mL). The resulted mixture was stirred at room temperature for 16 hours. The mixture was concentrated under reduced pressure to give target compound (crude). MS: M/e 454 (M+1)⁺.

### Step E: 2-(7-((2S,5R)-2,5-diethyl-4-(1-(3-fluoropyrazolo[1,5-a]pyrimidin-5-yl) ethyl)piperazin-1-yl)-4-methyl-5-oxo-4,5-dihydro-2H-pyrazolo[4,3-d]pyrimidin-2-yl)acetonitrile

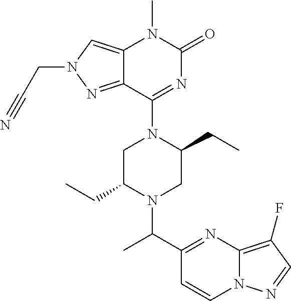 [SMILES: CC[C@H]1CN(C(C)c2ccn3ncc(F)c3n2)[C@H](CC)CN1c1nc(=O)n(C)c2cn(CC#N)nc12]

To a mixture of 7-((2S,5R)-2,5-diethyl-4-(1-(3-fluoropyrazolo[1,5-a]pyrimidin-5-yl)ethyl)piperazin-1-yl)-4-methyl-2,4-dihydro-5H-pyrazolo[4,3-d]pyrimidin-5-one (70 mg, crude) and K₂CO₃ (180 mg, 1.3 mmol) in DMF (2 mL) was added 2-chloroacetonitrile (48 mg, 0.65 mmol). The reaction was stirred at room temperature for 16 h. The reaction was diluted with water, extracted with EtOAc (70 mL), washed with brine, dried over Na₂SO₄, filtered, and concentrated. The resulting residue was purified by Prep-HPLC (Method A) to give the titled Compound A164a (14 mg, 21% for two steps) and Compound A164b (26 mg, 40% for two steps).

Compound A164a (the earlier peak): ¹HNMR (400 MHz, CD₃OD) δ 8.74 (d, J=7.2 Hz, 1H), 8.10 (d, J=3.2 Hz, 1H), 7.92 (d, J=6.8 Hz, 1H), 7.32-7.24 (m, 1H), 5.85-5.76 (m, 0.5H), 5.60-5.45 (m, 2,5H), 5.29-5.20 (m, 0.5H), 5.09-4.98 (m, 0.5H), 3.84-3.72 (m, 1.5H), 3.41 (s, 3H), 3.39-3.34 (m, 0.5H), 3.26-3.12 (m, 1H), 2.90-2.71 (m, 1H), 2.36-2.27 (m, 1H), 2.13-1.74 (m, 2H), 1.58-1.44 (m, 2H), 1.40 (d, J=6.8 Hz, 3H), 1.10-0.94 (m, 3H), 0.75 (t, J=7.6 Hz, 3H) ppm. MS: M/e 493 (M+1)⁺.

Compound A164b (the later peak): ¹HNMR (400 MHz, CD₃OD) δ 8.73 (d, J=7.6 Hz, 1H), 8.10 (d, J=2.8 Hz, 1H), 7.92 (d, J=9.6 Hz, 1H), 7.28-7.20 (m,1H), 5.80-5.68 (m, 0.5H), 5.63-5.45 (m, 2,5H), 5.24-5.16 (m, 0.5H), 5.07-4.98 (m, 0.5H), 4.05-3.92 (m, 1H), 3.65-3.54 (m, 0.5H), 3.41 (s, 1.5H), 3.40 (s, 1.5H), 3.22-3.14 (m, 0.5H), 3.05-2.83 (m, 2H), 2,59-2,44 (m, 1H), 2.25-1.92 (m, 2H), 1.69-1.56 (m, 1H), 1.51-1.38 (m, 4H), 0.98-0.88 (m, 3H), 0.83 (t, J=7.2 Hz, 1.5H), 0.77 (t, J=7.6 Hz, 1.5H) ppm. MS: M/e 493 (M+1)⁺.

### Compound A165: 2-(7-((2S,5R)-2,5-diethyl-4-(1-(3-fluoropyrazolo[1,5-a]pyridin-5-yl)ethyl)piperazin-1-yl)-4-methyl-5-oxo-4,5-dihydro-2H-pyrazolo[4,3-b]pyridin-2-yl)acetonitrile

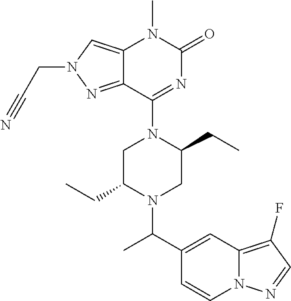 [SMILES: CC[C@H]1CN(C(C)c2ccn3ncc(F)c3c2)[C@H](CC)CN1c1nc(=O)n(C)c2cn(CC#N)nc12]

### Step A: 5-bromo-3-fluoropyrazolo[1.5-a]pyridine

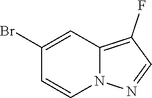 [SMILES: Fc1cnn2ccc(Br)cc12]

To a solution of 5-bromopyrazolo[1,5-a]pyridine (1.0 g, 5.0 mmol) in MeCN (20 mL) was added Select-F (1.8 g, 5.0 mmol) at room temperature and the mixture was stirred for 16 hours. The mixture was diluted with EtOAc (20 mL), washed with brine (10 mL×2), dried over Na₂SO₄, filtered and concentrated under reduced pressure. The resulting residue was purified by flash column chromatography to give the title compound (440 mg, 41%). ¹H NMR (400 MHz, CDCl₃) δ 8.13 (d, J=7.2 Hz, 1H), 7.80 (d, J=3.6 Hz, 1H), 7.68 (d, J=2.0 Hz, 1H), 6.77 (dd, J=7.6, 2.0 Hz, 1H) ppm. MS: M/e 215, 217 (M+1)⁺.

### Step B: 1-(3-fluoropyrazolo[1,5-a]pyridin-5-yl)ethan-1-one

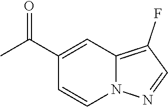

A mixture of 5-bromo-3-fluoropyrazolo[1,5-a]pyridine (420 mg, 1.94 mmol), tributyl(1-ethoxyvinyl)stannane (970 mg, 2.70 mmol) and Pd(PPh₃)₂Cl₂ (133 mg, 0.19 mmol) in toluene (5 mL) was stirred at 100° C. under N₂ for 16 hrs. The mixture was cooled and diluted with EA (10 mL), treated with HCl/Dioxane (4M, 3 mL), and stirred for 10 minutes. The resulted mixture was basified with aqueous solution of NaHCO₃ to pH-9 and washed with brine (10 mL×2), dried over Na₂SO₄, filtered and concentrated to dryness. The resulted residue was purified by flash column chromatography to give the title compound (300 mg, 87%). ¹H NMR (400 MHz, CDCl₃) δ 8.30 (d, .=7.2 Hz, 1H), 8.15 (s, 1H), 7.89 (d, J=3.6 Hz, 1H), 7.31 (dd, J=7.6, 1.6 Hz, 1H), 2.66 (s, 3H) ppm.

### Step C: 1-(3-fluoropyrazolo[1.5-a]pyridin-5-yl)ethan-1-ol

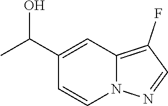

To a solution of 1-(3-fluoropyrazolo[1,5-a]pyridin-5-yl)ethan-1-one (300 mg, 1.68 mmol) in MeOH (3 mL) was added NaBH₄ (45 mg, 1,20 mmol) at room temperature and the mixture was stirred for 30 minutes. The mixture was concentrated and the residue was diluted with EtOAc (5 mL), washed with brine (2 mL×2), dried over Na₂SO₄, filtered and concentrated under reduced pressure to give the title compound (240 mg, 79%). ¹H NMR (400 MHz, DMSO-d₆) δ 8.49 (d, J=7.2 Hz, 1H), 8.04 (d, J=3.6 Hz, 1H), 7.51 (s, 1H), 6.85 (dd, J=7.2, 1.6 Hz, 1H), 5.41 (d, J=4.8 Hz, 1H), 4.83-4.69 (m, 1H), 1.34 (d, J=6.4 Hz, 3H) ppm. MS: M/e 181 (M+1)⁺.

### Step D: 2-(7-((2S,5R)-2,5-diethyl-4-(1-(3-fluoropyrazolo[1.5-a]pyridin-5-yl)ethyl)piperazin-1-yl)-4-methyl-5-oxo-4,5-dihydro-2H-pyrazolo[4,3-b]pyridin-2-yl)acetonitrile

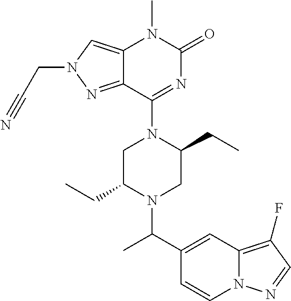 [SMILES: CC[C@H]1CN(C(C)c2ccn3ncc(F)c3c2)[C@H](CC)CN1c1nc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), 1-(3-fluoropyrazolo[1,5-a]pyridin-5-yl)ethan-1-ol (36 mg, 0.2 mmol), (cyanomethyl)trimethylphosphonium iodide (85 mg, 0.35 mmol) and DIPEA (65 mg, 0.5 mmol) in MeCN (1 mL) was stirred at 100° C. in a sealed tube for 16 hours. The mixture was diluted with EA (10 mL), washed with brine (5 mL×3), dried over Na₂SO₄, filtered and concentrated to dryness. The resulted residue was purified by column chromatography and further separated into Compound A165a (1.6 mg, 3.2%) and Compound A165b (2.0 mg, 4.0%) by Prep-HPLC (Method A).

Compound A165a (the earlier peak): ¹H NMR (400 MHz, CD₃OD) δ 8.38 (d, J=7.2 Hz, 1H), 7.93 (s, 1H), 7.88 (d, J=3.2 Hz, 1H), 7.51 (s, 1H), 7.02 (d, J=7.2 Hz, 1H), 5.58 (s, 1H), 5.47 (s, 2H), 5.33-5.01 (m, 1H), 4.57-3.97 (m, 1H), 3.87 (q, J=6.4 Hz, 1H), 3.44 (s, 3H), 3.36 (s, 1H), 3.02 (d, J=11.6 Hz, 1H), 2.90 (d, J=9.2 Hz, 1H), 2,50 (s, 1H), 2.24-2.06 (m, 1H), 1.92-1.76 (m, 1H), 1.70-1.51 (m, 2H), 1.39 (d, 0.1=6.0 Hz, 3H), 0.96 (t, J=7.2 Hz, 3H), 0.76 (t, J=7.2 Hz, 3H) ppm. MS: M/e 491 (M+1)”.

Compound A165b (the later peak): ¹H NMR (400 MHz, CD₃OD) δ 8.35 (d, J=7.2 Hz, 1H), 7.94 (s, 1H), 7.87 (d, J=3.4 Hz, 1H), 7.56 (s, 1H), 7.03 (d, J=7.2 Hz, 1H), 5.56 (s, 1H), 5.48 (s, 2H), 5.32-4.99 (m, 1H), 4.56-3.82 (m, 1H), 3.71 (q, 0.1=6.4 Hz, 1H), 3.53 (d, 0.1=13.2 Hz, 1H), 3.44 (s, 3H), 3.15 (d, J=10.8 Hz, 1H), 2.73 (d, J=8.8 Hz, 1H), 2,44 (d, J=12,4 Hz, 1H), 2.08-1.90 (m, 1H), 1.82-1.62 (m, 2H), 1.62-1.49 (m, 1H), 1.37 (d, J=6.4 Hz, 3H), 1.04 (t, J=7.2 Hz, 3H), 0.68 (t, J=7.2 Hz, 3H) ppm. MS: M/e 491 (M+1)⁺.

### Compound A166: 2-(7-((2S,5R)-5-ethyl-2-methyl-4-(1-(pyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile

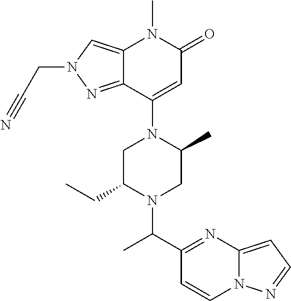 [SMILES: CC[C@@H]1CN(c2cc(=O)n(C)c3cn(CC#N)nc23)[C@@H](C)CN1C(C)c1ccn2nccc2n1]

To a solution of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (50 mg, 0.15 mmol) in CH₃CN (5 mL) and was added 1-(pyrazolo[1,5-a]pyrimidin-5-yl)ethan-1-ol (77 mg, 0.46 mmol), (cyanomethyl)trimethylphosphonium iodide (154 mg, 0.63 mmol) and DIPEA(205 mg, 1.5 mmol).The resulting mixture was stirred at 105° C. overnight. The reaction solvent was removed under reduce pressure. The resulting residue was purified by flash column chromatography to give the titled compound(crude), which was further separated into Compound A166a (10 mg, 13%) and Compound A166b (6 mg, 8%) by Prep-HPLC (Method B).

Compound A166a (the earlier peak): ¹H NMR (400 MHz, DMSO-d₆) δ 9.05 (d, J=7.2 Hz, 1H), 8.17 (d, .=2.1 Hz, 1H), 7.99 (s, 1H), 7.16 (d, J=7.3 Hz, 1H), 6.66 (d, J=1.4 Hz, 1H), 5.60 (s, 2H), 5.43 (s, 1H), 4.75-4.22 (m, 2H), 3.91 (q, J=6.4 Hz, 1H), 3.27 (s, 3H), 3.23 (s, 1H), 2.93 (d, J=8.1 Hz, 1H), 2.72 (d, J=11.1 Hz, 1H), 2,44 (d, J=8.3 Hz, 1H), 1.66-1.45 (m, 2H), 1.34 (d, J=6.6 Hz, 3H), 1,24 (d, J=6.3 Hz, 3H), 0.72 (t, J=7.3 Hz, 3H) ppm. MS: M/e 460 (M+1)⁺.

Compound A166a (the later peak): ¹H NMR (400 MHz, DMSO-d₆) δ 9.05 (d, J=7.3 Hz, 1H), 8.17 (d, J=2.3 Hz, 1H), 7.99 (s, 1H), 7.18 (d, J=7.3 Hz, 1H), 6.62 (d, J=1.6 Hz, 1H), 5.62 (s, 2H), 5.41 (s, 1H), 5.01-4.55 (m, 1H), 4.41-4.06 (m, 1H), 3.73 (q, J=6.5 Hz, 1H), 3.44 (d, J=10.9 Hz, 1H), 3.27 (s, 3H), 3.04 (d, J=10.3 Hz, 1H), 2.72 (dd, J=12.0, 3.8 Hz, 1H), 2.14 (d, J=10.0 Hz, 1H), 1.69-1.36 (m, 2H), 1.32 (d, J=6.6 Hz, 3H), 1.12 (d, J=6.4 Hz, 3H), 0.97 (t, J=7.3 Hz, 3H) ppm. MS: M/e 460 (M+1)”.

### Compound A167: 2-(7-((2S,5R)-4-(1-(3-chloroimidazo[1,2-b]pyridazin-6-yl)ethyl)-2,5diethylpiperazin-1-yl)-4-methyl-5-oxo-4,5-dihydro-2H-pyrazolo[4,3-b]pyridin-2-yl)acetonitrile

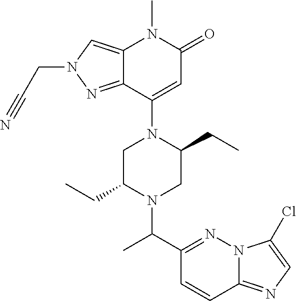 [SMILES: CC[C@H]1CN(C(C)c2ccc3ncc(Cl)n3n2)[C@H](CC)CN1c1cc(=O)n(C)c2cn(CC#N)nc12]

### Step A: 6-bromo-3-chloroimidazo[1,2-b]pyridazine

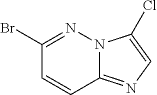 [SMILES: Clc1cnc2ccc(Br)nn12]

To a solution of 6-bromoimidazo[1,2-b]pyridazine (990 mg, 5 mmol) in acetonitrile (10 mL) and acetic acid(1 mL) was added NCS (665 mg, 5 mmol). The reaction mixture was stirred at 60° C. overnight. The mixture was added satd NaHCO₃ (100 mL) and extracted with EtOAc (20 mL×3). The organic phase was dried with Na₂SO₄ and concentrated by using a rotary evaporator to give a residue. The resulting residue was purified by flash column chromatography (EA:PE=0-50% ) to give the titled compound (1 g, crude). MS: M/e 232 (M+1)⁺.

### Step B: 1-(3-chloroimidazo[1,2-b]pyridazin-6-yl)ethan-1-one

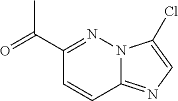

To a solution of 6-bromo-3-chloroimidazo[1,2-b]pyridazine (1 g, 4.3 mmol) in DMF (15 mL) was added Pd(PPh₃)₂Cl₂ (301 mg, 0.43 mmol) and tributyl(1-ethoxyvinyl) stannane (1.7 g, 4.7 mmol). The reaction mixture was protected by N₂ atmosphere and stirred at 100° C. overnight. The mixture was added HCl (2 mL, 1N) and stirred at RT for 30 mins. The mixture was added saturated NaHCO₃ aqueous solution (50 mL) and extracted with EtOAc (10 mL×3). The organic phase was dried with Na₂SO₄ and concentrated by using a rotary evaporator to give a residue. The resulting residue was purified by flash column chromatography (EA:PE=0-30%) to give the titled compound (350 mg, 41.7%). MS: M/e 196 (M+1)⁺.

### Step C: 1-(3-chloroimidazole,2-b]pyridazin-6-yl)ethan-1-ol

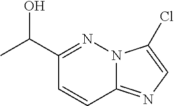

To a solution of 1-(3-chloroimidazo[1,2-b]pyridazin-6-yl)ethan-1-one (350 mg, 1.8 mmol) in MeOH was added NaBH₄ (54 mg, 1.4 mmol). The reaction mixture was stirred at RT for 30 mins. The mixture was added H₂O (10 mL) and extracted with EtOAc (5 mL×2). The organic phase was dried with Na₂SO₄ and concentrated by using a rotary evaporator to give a residue. The resulting residue (300 mg, crude) was used in the next step directly. MS: M/e 198 (M+1)⁺.

### Step D: 2-(7-((2S,5R)-4-(143-chloroimidazole,2-b]pyridazin -6-yl)ethyl)-2,5-diethylpiperazin-1-yl)-4-methyl-5-oxo-4,5-dihydro-2H-pyrazolo[4,3-b]pyridin-2-yl)acetonitrile

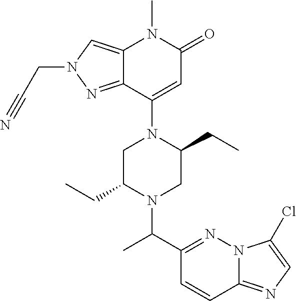 [SMILES: CC[C@H]1CN(C(C)c2ccc3ncc(Cl)n3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), 1-(3-chloroimidazo[1,2-b]pyridazin-6-yl)ethan-1-ol (23 mg, 0.12 mmol) and (cyanomethyl)trimethylphosphonium iodide (97 mg, 0.4 mmol) in CH₃CN (2 mL) was added DIPEA (51 mg, 0.4 mmol). The reaction mixture was sealed in a bottle and heated at 100° C. for 16 hours, and then cooled to room temperature, diluted with water(30 mL), extracted with EtOAc (10 mL×2). The combined organic layers were washed with brine, dried over Na₂SO₄, filtered, and concentrated to dryness. The resulting residue was purified and separated into Compound A167a (1,2 mg, 2.3%) and Compound A167b (1.6 mg, 3.1%) by Prep-HPLC (Method A).

Compound A167a (the earlier peak): ¹H NMR (400 MHz, CDCl₃) δ 8.37-7.68 (m, 2H), 7.46 (d, J=52.1 Hz, 2H), 5.64 (s, 1H), 5.19-5.03 (m, 3H), 4.42 (s, 2H), 3.98 (s, 1H), 3.43 (s, 3H), 3.08 (s, 1H), 2.84 (s, 1H), 2.16 (d, J=66.3 Hz, 2H), 1.82 (s, 3H), 1.42 (s, 3H), 1.18 (s, 1H), 0.98 (s, 2H), 0.62 (s, 3H) ppm. MS: M/e 508 (M+1)⁺.

Compound A167b (the later peak): ¹H NMR (400 MHz, CDCl₃) δ 7.93 (d, J=9.4 Hz, 1H), 7.68 (s, 1H), 7.45-7.27 (m, 2H), 5.61 (s, 1H), 5.18-5.05 (m, 2H), 4.58 (s, 2H), 4.12 (d, J=6.8 Hz, 1H), 3.42 (s, 3H), 3.24 (d, J=11.1 Hz, 1H), 2.94 (s, 2H), 2.34 (s, 1H), 2.23-1.95 (m, 2H), 1.75 (d, J=6.6 Hz, 1H), 1.65 (d, J=6.9 Hz, 1H), 1.45 (d, J=6.6 Hz, 3H), 0.90 (t, J=7.2 Hz, 3H), 0.72 (dd, J=33.7, 26.5 Hz, 3H) ppm. MS: M/e 508 (M+1)⁺.

### Compound A168: 2-(7-((2S,5R)-2,5-diethyl-4-(1-(3-fluoroimidazo[1,2-b]pyridazin-6-yl)ethyl)piperazin-1-yl)-4-methyl-5-oxo-4,5-dihydro-2H-pyrazolo[4,3-b]pyridin-2-yl)acetonitrile

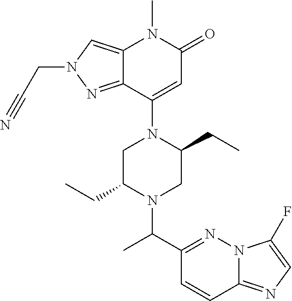 [SMILES: CC[C@H]1CN(C(C)c2ccc3ncc(F)n3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (32 mg, 0.1 mmol) and 1-(3-fluoroimidazo[1,2-b]pyridazin-6-yl)ethan-1-ol (18 mg, 0.1 mmol), (cyanomethyl) trimethyl phosphonium iodide (75 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EtOAc (70 mL×2), washed with brine, dried over Na₂SO₄, filtered, and concentrated to dryness. The resulting residue was purified by Prep-TLC (EtOAc) and further separated into Compound A168a (3 mg, 6%) and Compound A168b (4 mg, 8%) by Prep-HPLC (Method A).

Compound A168a (the earlier peak): ¹H NMR (400 MHz, CD₃OD) δ 7.97-7.91 (m, 2H), 7.53 (d, J=9.6 Hz, 1H), 7.42 (d, J=6.4 Hz, 1H), 5.57 (s, 1H), 5.48 (s, 2H), 5.02-4.92 (m, 1H), 4.82-4.72 (m, 1H), 3.96-3.86 (m, 1H), 3.62-3.50 (m, 1H), 3.44 (s, 3H), 3.20-3.10 (m, 1H), 2.90-2.79 (m, 1H), 2.37-2.29 (m, 1H), 1.95-1.50 (m, 4H), 1.44 (d, J=6.8 Hz, 3H), 1.04 (t, J=7.2 Hz, 3H), 0.67 (t, J=7.2 Hz, 3H) ppm. MS: M/e 492 (M+1)⁺.

Compound A168b (the later peak): ¹H NMR (400 MHz, CD₃OD) δ 7.97-7.91 (m, 2H), 7.47 (d, J=10.0 Hz, 1H), 7.41 (d, J=6.4 Hz, 1H), 5.57 (s, 1H), 5.46 (s, 2H), 5.00-4.92 (m, 1H), 4.82-4.72 (m, 1H), 4.14-4.06 (m, 1H), 3.44 (s, 3H), 3.39-3.35 (m, 1H), 3.01-2.94 (m, 2H), 2,50-2,44 (m, 1H), 2.08-1.99 (m, 1H), 1.87-1.76 (m, 1H), 1.69-1.59 (m, 2H), 1.47 (d, 0.1 =6.8 Hz, 3H), 0.91 (t, J=7.6 Hz, 3H), 0.80 (t, J=8.0 Hz, 3H) ppm. MS: M/e 492 (M+1)⁺.

### Compound A169: 2-(7-((2S,5R)-2,5-diethyl-4-(1-(3-fluoro-2-methylpyrazolo[1,5-a]pyridin-5-yl)ethyl)piperazin-1-yl)-4-methyl-5-oxo-4,5-dihydro-2H-pyrazolo[4,3-b]pyridin-2-yl)acetonitrile

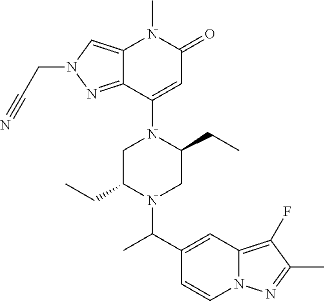 [SMILES: CC[C@H]1CN(C(C)c2ccn3nc(C)c(F)c3c2)[C@H](CC)CN1c1cc(=O)n(C)c2cn(CC#N)nc12]

### Step A: 1-(3-fluoro-2-methylpyrazolo[1,5-a]pyridin-5-yl)ethan-1-one

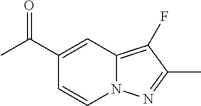

To a solution of 1-(2-methylpyrazolo[1,5-a]pyridin-5-yl)ethan-1-one (80 mg, 0.46 mmol) in MeCN (3 mL) was added Select-F (350 mg, 0.99 mmol) at 0° C. and the mixture was stirred at room temperature for 30 minutes. The mixture was diluted with EtOAc (10 mL), washed with brine (5 mL×2), dried over Na₂SO₄, filtered and concentrated under reduced pressure. The resulting residue was purified by flash column chromatography to give the title compound (35 mg, 40%). MS: M/e 193 (M+1)⁺.

### Step B: 1-(3-fluoro-2-methylpyrazolo[1,5-a]pyridin-5-yl)ethan-1-ol

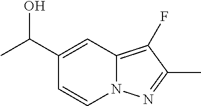

To a solution of 1-(3-fluoro-2-methylpyrazolo[1,5-a]pyridin-5-yl)ethan-1-one (35 mg, 0.18 mmol) in MeOH (2 mL) was added NaBH₄ (7 mg, 0.18 mmol) at room temperature and the mixture was stirred for 2 hours. The mixture was concentrated and the residue was diluted with EtOAc (10 mL), washed with brine (5 mL×2), dried over Na₂SO₄, filtered and concentrated under reduced pressure. The resulting residue was purified by flash column chromatography to give the title compound (24 mg, 68%). MS: M/e 195 (M+1)⁺.

### Step C: 2-(7-((2S,5R)-2.-diethyl-4-(1-(3-fluoro-2-methylpyrazolo[1,5-a]pyridin-5-yl)ethyl)piperazin-1-yl)-4 methyl-5-oxo-4,5-dihydro-2H-pyrazolo[4,3-b]pyridin-2-yl)acetonitrile

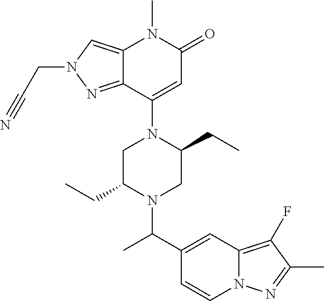 [SMILES: CC[C@H]1CN(C(C)c2ccn3nc(C)c(F)c3c2)[C@H](CC)CN1c1cc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), 1-(3-fluoro-2-methylpyrazolo[1,5-a]pyridin-5-yl)ethan-1-ol (24 mg, 0.12 mmol), (cyanomethyl)trimethylphosphonium iodide (85 mg, 0.35 mmol) and DIPEA (65 mg, 0.5 mmol) in MeCN (1 mL) was stirred at 100° C. in a sealed tube for 16 hours. The mixture was diluted with EA (10 mL), washed with brine (5 mL×3), dried over Na₂SO₄, filtered and concentrated to dryness. The resulted residue was purified by column chromatography and further separated by Prep-HPLC (Method A) to give Compound A169a (0.08 mg, 0.16%) and Compound A169b (0.10 mg, 0.20%).

Compound A169a (the earlier peak): ¹H NMR (400 MHz, CD₃OD) δ 8.52 (s, 3H) (HCOOH), 8.26 (d, J=7.2 Hz, 1H), 7.92 (s, 1H), 7.40 (s, 1H), 6.93 (d, J=6.8 Hz, 1H), 5.57 (s, 1H), 5.46 (s, 2H), 5.13-5.07 (m, 1H), 4.22-4.10 (m, 1H), 3.86-3.82 (m, 1H), 3.43 (s, 3H), 3.03-2.97 (m, 1H), 2.92-2.88 (m, 1H), 2,53-2,46 (m, 1H), 2,41 (s, 3H), 2.18-2.12 (m, 1H), 2.07-2.01 (m, 1H), 1.88-1.81 (m, 1H), 1.62-1.57 (m, 2H), 1.38 (d, J=6.4 Hz, 3H), 0.95 (t, J=7.2 Hz, 3H), 0.75 (t, J=7.2 Hz, 3H) ppm. MS: M/e 505 (M+1)⁺.

Compound A169b (the later peak): ¹H NMR (400 MHz, CD₃OD) δ 8.51 (s, 2H) (HCOOH), 8.24 (d, J=7.6 Hz, 1H), 7.93 (s, 1H), 7.45 (s, 1H), 6.94 (d, J=6.0 Hz, 1H), 5.56 (s, 1H), 5.47 (s, 2H), 5.16-5.04 (m, 1H), 4.40-4.25 (m, 1H), 3.56-3.50 (m, 1H), 3.44 (s, 3H), 3.15-3.10 (m, 1H), 2.75-2.69 (m, 1H), 2,48-2.36 (m, 4H), 2.05-2.01 (m, 1H), 1.75-1.65 (m, 2H), 1.62-1.52 (m, 2H), 1.36 (d, J=6.8 Hz, 3H), 1.03 (t, J=7.2 Hz, 3H), 0.68 (t, J=7.2 Hz, 3H) ppm. MS: M/e 505 (M+1)⁺.

### Compound A170: 2-(7-((2S,5R)-2,5-diethyl-4-(1-(3-fluoro-2-methylpyrazolo[1,5-a]pyrimidin-5-yl)ethyl)piperazin-1-yl)-4-methyl-5-oxo-4,5-dihydro-2H-pyrazolo[4,3-d]pyrimidin-2-yl)acetonitrile

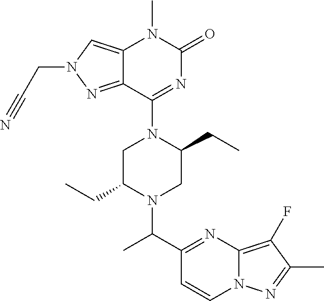 [SMILES: CC[C@H]1CN(C(C)c2ccn3nc(C)c(F)c3n2)[C@H](CC)CN1c1nc(=O)n(C)c2cn(CC#N)nc12]

### Step A: 4-fluoro-3-methyl-1H-pyrazol-5-amine

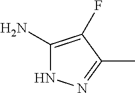

To a solution of 3-methyl-1H-pyrazol-5-amine (0.5 g, 5.1 mmol) in CH₃CN (20 mL) were added 1-Chloromethyl-4-fluoro-1,4-diazoniabicyclo[2.2.2]octane bis(tetrafluoroborate) (1.6 g, 4.6 mmol) was stirred at room temperature under N₂ for 16 hours. The solution was purified by flash column chromatography to give the titled compound (0.41 g). MS: M/e 116 (M+1)⁺

### Step B: 3-fluoro-2-methylpyrazolo[1,5-a]pyrimidin-5 (4H)-one

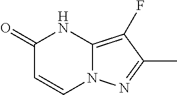

A mixture of 4-fluoro-3-methyl-1H-pyrazol-5-amine (0.41 g, 3.5 mmol), ethyl (E)-3-ethoxyacrylate (1 g, 7.1 mmol) and Cs₂CO₃ (2.3 g, 7.1 mmol) in DMF (15 mL) was stirred at 110° C. under N₂ for 16 hours. The solution was collected by filtration, then added acetic acid (5 mL). The organic layer was concentrated to dryness. The resulting residue was purified by flash column chromatography to give the titled compound (0.09 g, 15%). MS: M/e 168 (M+1)⁺

### Step C: 5-chloro-3-fluoro-2-methylpyrazolo[1,5-a]pyrimidine

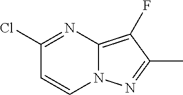

To a solution of 3-fluoro-2-methylpyrazolo[1,5-a]pyrimidin-5 (4H)-one (0.09 g, 0.54 mmol) in CH₃CN (3 mL) were added POCl₃ (0.16 g, 1 mmol). The mixture was stirred at 90° C. for 3 hours. The reaction was concentrated and poured into saturated NaHCO₃ solution, extracted with DCM. The combined organic layers were washed with brine, dried over Na₂SO₄ and concentrated to dryness. The resulting residue was purified by flash column chromatography to give the titled compound (0.07 g, 70%). MS: M/e 186 (M+1)⁺.

### Step D: 1-(3-fluoro-2-methylpyrazolo[1,5-a]pyrimidin-5-yl)ethan-1-one

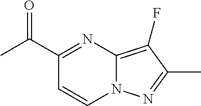

A mixture of 5-chloro-3-fluoro-2-methylpyrazolo[1,5-a]pyrimidine (0.07 g, 0.38 mmol), tributyl(1-ethoxyvinyl)stannane (0.16 g, 0.45 mmol) and Pd(PPh₃)₂Cl₂ (27 mg, 0.038 mmol) in DMF (2 mL) was stirred at 100° C. under N₂ for 16 hours. The solution was diluted with ethyl acetate and washed with water. The organic layer was concentrated to dryness. The resulting residue was purified by flash column chromatography to give the titled compound (0.06 g, 82%). MS: M/e 194 (M+1)⁺.-

### Step E: 1-(3-fluoro-2-methylpyrazolo[1,5-a]pyrimidin-5-yl)ethan-1-ol

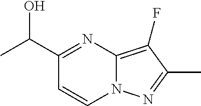

To a solution of 1-(3-fluoro-2-methylpyrazolo[1,5-a]pyrimidin-5-yl)ethan-1-one (0.06 g, 0.31 mmol) in MeOH (5 mL) was added NaBH₄ (7 mg, 0.18 mmol) at room temperature and the resulting mixture was stirred at room temperature for 5 mins. The reaction mixture was diluted with DCM and washed with water, dried over Na₂SO₄ and concentrated to give the titled compound (150 mg). MS: M/e 196 (M+1)⁺.

### Step F: tert-butyl (2S,5R-2,5-diethyl-4-(1-(3-fluoro-2-methylpyrazolo[1,5-a]pyrimidin-5-yl)ethyl)piperazine-1-carboxylate

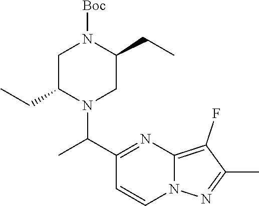 [SMILES: CC[C@H]1CN(C(C)c2ccn3nc(C)c(F)c3n2)[C@H](CC)CN1C(=O)OC(C)(C)C]

To a solution of tert-butyl (2S,5R)-2,5-diethylpiperazine-1-carboxylate (150 mg, 0.62 mmol), 1-(3-fluoro-2-methylpyrazolo[1,5-a]pyrimidin-5-yl)ethan-1-ol (120 mg, 0.62 mmol) and DIPEA (400 mg, 3.1 mmol) in CH₃CN (6 mL) was added (cyanomethyl)trimethylphosphonium iodide (450 mg, 1.86 mmol). The mixture was stirred at 103° C. in a sealed bottle for 18 hours. The reaction was cooled to room temperature, diluted by adding water, extracted with EtOAc (60 mL×2), washed with brine, dried over Na₂SO₄, filtered and concentrated to dryness. The residue was purified by flash column chromatography to give the titled compound (145 mg, 55%). MS: M/e 420 (M+1)⁺.

### Step G: 5-(1-((2R,5S)-2,5-diethylpiperazin-1-yl)ethyl)-3-fluoro-2-methylpyrazolo[1,5-a]pyrimidine

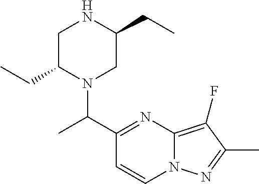 [SMILES: CC[C@H]1CN(C(C)c2ccn3nc(C)c(F)c3n2)[C@H](CC)CN1]

To a solution of tert-butyl (2S,5R)-2,5-diethyl-4-(1-(3-fluoro-2-methylpyrazolo[1,5-a]pyrimidin-5-yl)ethyl)piperazine-1-carboxylate (145 mg, 0.346 mmol) in DCM (10 mL) was added TFA (1 mL). The resulted mixture was stirred at room temperature for 16 hours. The reaction mixture was quenched with water (50 mL), extracted with DCM (60 mL). The aqueous layer was collected, basified with aq. NaHCO₃, then extracted with CH₂Cl₂/MeOH (10/1, 60 mL×3). The combined organic layers were dried over Na₂SO₄, concentrated to give the titled compound (80 mg, 72%). MS: M/e 320 (M+1)⁺.

### Step H: 7-((2S,5R)-2,5-diethyl-4-(1-(3-fluoro-2-methylpyrazolo[1,5-a]pyrimidin-5-yl)ethyl)piperazin-1-yl-4-methyl-2-(tetrahydro-2H-pyran-2-yl-2,4-dihydro-5H-pyrazolo[4,3-d]pyrimidin-5-one

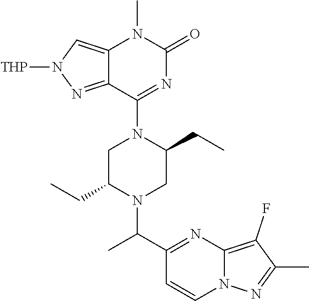

To a mixture of 4-methyl-2-(tetrahydro-2H-pyran-2-yl)-2,4-dihydro-5H-pyrazolo[4,3-d]pyrimidine-5,7 (6H)-dione (125 mg, 0.5 mmol) in CH₃CN (3 mL) was added DIPEA (129 mg, 1 mmol), followed by POCl₃ (153 mg, 1 mmol) and catalyst amount of DMF. The reaction was stirred at 80° C. for 1.5 hours. The reaction was cooled to room temperature, quenched with ice-water, extracted with EtOAc (60 mL×2), washed with brine, dried over Na₂SO₄, filtered, and concentrated.

To a mixture of 5-(1-((2R.,5S)-2,5-diethylpiperazin-1-yl)ethyl)-3-fluoro-2-methylpyrazolo[1,5-a]pyrimidine (80 mg, 0.25 mmol) in CH₃CN (5 mL) was added DIEA (129 mg, 1 mmol), follow by a solution of the above residue in CH₃CN (2 mL). The resulting mixture was heated at 90° C. for 3 hours. The reaction was quenched with water, extracted with EtOAc (80 mL), washed with brine, dried over Na₂SO₄, filtered, and concentrated. The residue was purified by flash column chromatography to give target compound (90 mg, 35% for two steps). MS: M/e 552 (M+1)⁺.

### Step I: 7-((2S,5R)-2,5-diethyl-4-(1-(3-fluoro-2-methylpyrazolo[1,5-a]pyrimidin-5-yl)ethyl)piperazin-1-yl-4-methyl-2,4-dihydro-5H-pyrazolo[4,3-d]pyrimidin-5-one

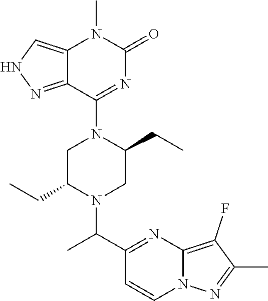 [SMILES: CC[C@H]1CN(C(C)c2ccn3nc(C)c(F)c3n2)[C@H](CC)CN1c1nc(=O)n(C)c2c[nH]nc12]

To a solution of 7-((2S,5R)-2,5-diethyl-4-(1-(3-fluoro-2-methylpyrazolo[1,5-a]pyrimidin-5-yl)ethyl)piperazin-1-yl)-4-methyl-2-(tetrahydro-2H-pyran-2-yl)-2,4-dihydro-5H-pyrazolo[4,3-d]pyrimidin-5-one (90 mg, 0.163 mmol) in DCM (2 mL) was added TFA (6 mL). The resulted mixture was stirred at room temperature for 16 hours. The mixture was concentrated under reduced pressure to give target compound (90 mg, crude). MS: M/e 468 (M+1)⁺.

### Step J: 2-(7-((2S,5R)-2,5-diethyl-4-(1-(3-fluoro-2-methylpyrazolo[1,5-a]pyrimidin -5-yl)ethyl)piperazin-1-yl)-4-methyl-5-oxo-4,5-dihydro-2H-pyrazolo[4,3-d]pyrimidin-2-yl)acetonitrile

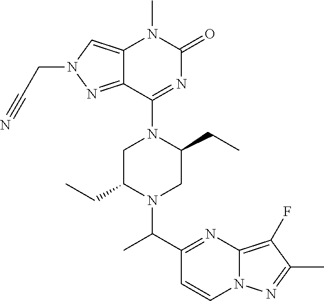 [SMILES: CC[C@H]1CN(C(C)c2ccn3nc(C)c(F)c3n2)[C@H](CC)CN1c1nc(=O)n(C)c2cn(CC#N)nc12]

To a mixture of 7-((2S,5R)-2,5-diethyl-4-(1-(3-fluoro-2-methylpyrazolo[1,5-a]pyrimidin-5-yl)ethyl)piperazin-1-yl)-4-methyl-2,4-dihydro-5H-pyrazolo[4,3-d]pyrimidin-5-one (90 mg, crude) and K₂CO₃ (225 mg, 1.63 mmol) in DMF (4 mL) was added 2-chloroacetonitrile (61 mg, 0.81 mmol). The reaction was stirred at room temperature for 16 hours. The reaction was diluted with water, extracted with EtOAc (70 mL×2), washed with brine, dried over Na₂SO₄, filtered, and concentrated. The resulting residue was purified and separated into Compound A170a (7 mg, 8.5% for two steps) and Compound A170b (15 mg, 18% for two steps) by Prep-HPLC (Method A).

Compound A170a (the earlier peak): ¹HNMR (400 MHz, CD₃OD) δ 8.64 (d, J=7.2 Hz, 1H), 7.91 (d, J=6.8 Hz, 1H), 7.24-7.15 (m, 1H), 5.85-5.74 (m, 0.5H), 5.60-5.45 (m, 2,5H), 5.29-5.19 (m, 0.5H), 5.08-4.98 (m, 0.5H), 3.82-3.68 (m, 1.5H), 3.41 (s, 3H), 3.39-3.34 (m, 0.5H), 3.26-3.10 (m, 1H), 2.91-2.71 (m, 1H), 2,45 (s, 3H), 2.36-2.27 (m, 1H), 2.12-1.71 (m, 2H), 1.60-1.44 (m, 2H), 1.38 (d, J=6.4 Hz, 3H), 1.10-0.94 (m, 3H), 0.75 (t, J=7.6 Hz, 3H) ppm. MS: M/e 507 (M+1)⁺.

Compound A170b (the later peak): ¹HNMR (400 MHz, CD₃OD) δ 8.63 (d, J=7.6 Hz, 1H), 7.91 (d, J=9.2 Hz, 1H), 7.20-7.08 (m,1 H), 5.80-5.68 (m, 0.5H), 5.63-5.45 (m, 2,5H), 5.26-5.14 (m, 0.5H), 5.08-4.98 (m, 0.5H), 4.01-3.89 (m, 1H), 3.66-3.54 (m, 0.5H), 3.41 (s, 3H), 3.22-3.14 (m, 0.5H), 3.05-2.80 (m, 2H), 2.60-2,45 (m, 4H), 2.15-1.90 (m, 2H), 1.69-1.55 (m, 1H), 1.51-1.37 (m, 4H), 0.98-0.88 (m, 3H), 0.83 (t, J=7.6 Hz, 1.5H), 0.77 (t, J=8.0 Hz, 1.5H) ppm. MS: M/e 507 (M+1)Y.

### Compound A171: 2-(7-((2S,5R)-5-ethyl-2-methyl-4-(1-(2-methylpyrazolo[1,5-a]pyrimidin-5-yl)propyl)piperazin-1-yl)-4-methyl-5-oxo-4,5-dihydro-2H-pyrazolo[4,3-b]pyridin-2-yl)acetonitrile

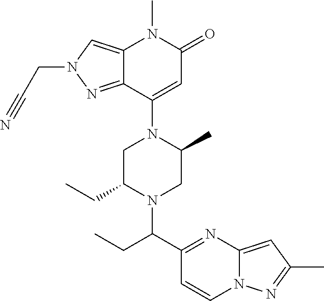 [SMILES: CCC(c1ccn2nc(C)cc2n1)N1C[C@H](C)N(c2cc(=O)n(C)c3cn(CC#N)nc23)C[C@H]1CC]

### Step A: 1-(2-methylpyrazolo[1.5-a]pyrimidin-5-yl)propan-1-one

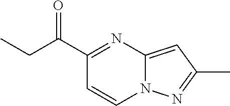

To a solution of 2-methylpyrazolo[1,5-a]pyrimidine-5-carbonitrile (0.7 g, 4.4 mmol) in THF (30 mL) were added CuBr (0.95 g, 6.6 mmol) was stirred at room temperature for 20 mins. Then added EtMgBr (5.1 mL, 3M in ethoxyethane, 15.5 mmol) and the resulting mixture was stirred at −10° C. for 10 mins The solution was quenched by saturated NH₄Cl solution, extracted with ethyl acetate. The combined organic layers were washed with brine, dried over Na₂SO₄ and the resulting residue was purified by purified by flash column chromatography to give the titled compound (0.27 g, 32%). MS: M/e 190 (M+1)⁺.

### Step B: 1-(2-methylpyrazolo[1,5-a]pyrimidin-5-yl)propan-1-ol

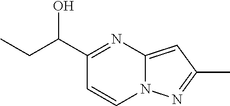

To a solution of 1-(2-methylpyrazolo[1,5-a]pyrimidin-5-yl)propan-1-one (0.27 g, 1.4 mmol) in MeOH (5 mL) was added NaBH₄ (32 mg, 0.85 mmol) at room temperature and the resulting mixture was stirred at room temperature for 5 mins. The reaction mixture was diluted with DCM and washed with water, dried over Na₂SO₄, concentrated to give the titled compound (250 mg). MS: M/e 192 (M+1)⁺.

### Step C: 2-(7-((2S,5R)-5-ethyl-2-methyl-4-(1-(2-methylpyrazolo[1,5-a]pyrimidin-5-yl)propyl)piperazin-1-yl)-4-methyl-5-oxo-4,5-dihydro-2H-pyrazolo[4,3-b]pyridin-2-yl)acetonitrile

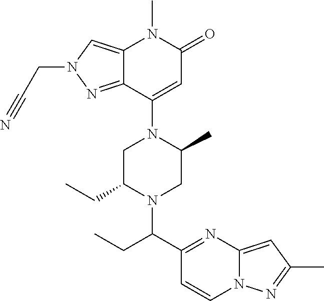 [SMILES: CCC(c1ccn2nc(C)cc2n1)N1C[C@H](C)N(c2cc(=O)n(C)c3cn(CC#N)nc23)C[C@H]1CC]

To a solution of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (50 mg, 0.15 mmol) in CH₃CN (5 mL) and was added 1-(2-methylpyrazolo[1,5-a]pyrimidin-5-yl)propan-1-ol (92 mg, 0.45 mmol), (cyanomethyl)trimethylphosphonium iodide (146 mg, 0.6 mmol) and DIPEA(197 mg, 1.5 mmol).The resulting mixture was stirred at 105° C. overnight. The reaction solvent was removed under reduce pressure. The resulting residue was purified by flash column chromatography to give the titled compound(crude), which was further separated into Compound A171a (4 mg, 5%) and Compound A171b (2 mg, 2%) by Prep-HPLC (Method B).

Compound A171a (the earlier peak): ¹H NMR (400 MHz, DMSO-d₆) δ 8.91 (d, J=7.2 Hz, 1H), 7.98 (s, 1H), 6.99 (d, J=7.2 Hz, 1H), 6.46 (s, 1H), 5.60 (s, 2H), 5.39 (s, 1H), 4.74-4.21 (m, 2H), 3.66 (dd, J=8.5, 5.2 Hz, 1H), 3.27 (s, 3H), 3.24 (d, J=13.6 Hz, 1H), 2.96 (dd, J=11.9, 4.2 Hz, 1H), 2.72 (dd, J=11.9, 3.8 Hz, 1H), 2,45 (s, 1H), 2,41 (s, 3H), 1.96-1.41 (m, 4H), 1,20 (d, J=6.3 Hz, 3H), 0.78-0.65 (m, 6H) ppm. MS: M/e 488 (M+1)⁺.

Compound A171b (the later peak): :H NMR (400 MHz, DMSO-d⁶) δ 8.91 (d, J=7.2 Hz, 1H), 7.99 (s, 1H), 7.03 (d, J=7.2 Hz, 1H), 6.42 (s, 1H), 5.61 (s, 2H), 5.40 (s, 1H), 5.04-4.01 (m, 2H), 3.50 (dd, J=9.6, 4.2 Hz, 1H), 3.40 (d, J=11.9 Hz, 1H), 3.27 (s, 3H), 3.04 (d, J=9.7 Hz, 1H), 2.76 (dd, J=12.0, 3.5 Hz, 1H), 2,40 (s, 3H), 2.11 (d, J=10.4 Hz, 1H), 1.97-1.35 (m, 4H), 1.11 (d, J=6.4 Hz, 3H), 0.96 (t, J=7.3 Hz, 3H), 0.68 (t, J=7.3 Hz, 3H) ppm. MS: M/e 488 (M+1)⁺.

Compound A172: 2-(7-((2S,5R)-5-ethyl-2-methyl-4-(1-(2-methylpyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile

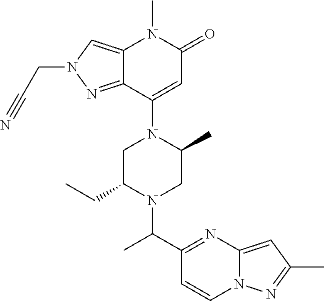 [SMILES: CC[C@@H]1CN(c2cc(=O)n(C)c3cn(CC#N)nc23)[C@@H](C)CN1C(C)c1ccn2nc(C)cc2n1]

To a solution of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (50 mg, 0.15 mmol) in CH₃CN (5 mL) and was added 1-(2-methylpyrazolo[1,5-a]pyrimidin-5-yl)ethan-1-ol (84 mg, 0.45 mmol), (cyanomethyl)trimethylphosphonium iodide (146 mg, 0.6 mmol) and DIPEA(197 mg, 1.5 mmol).The resulting mixture was stirred at 105° C. overnight. The reaction solvent was removed under reduce pressure. The resulting residue was purified by flash column chromatography to give the titled compound(crude), which was further separated into Compound A172a (11 mg, 14%) and Compound A172b (9 mg, 12%) by Prep-HPLC (Method B).

Compound A172a (the earlier peak): ¹H NMR (400 MHz, DMSO-s) δ 8.89 (d, J=7.2 Hz, 1H), 7.96 (s, 1H), 7.04 (d, J=7.1 Hz, 1H), 6.42 (s, 1H), 5.58 (s, 2H), 5.41 (s, 1H), 4.74-4.10 (m, 2H), 3.91-3.79 (m, 1H), 3.26 (s, 3H), 3.21 (s, 1H), 2.90 (d, J=8.1 Hz, 1H), 2.69 (d, J=11.2 Hz, 1H), 2,42 (s, 1H), 2.39 (s, 3H), 1.58-1.46 (m, 2H), 1.31 (d, J=6.5 Hz, 3H), 1,22 (d, J=6.3 Hz, 3H), 0.70 (t, J=7.2 Hz, 3H) ppm. MS: M/e 474 (M+1)⁺.

Compound A172b (the later peak): ¹H NMR (400 MHz, DMSO-d₆) δ 8.89 (d, J=7.2 Hz, 1H), 7.97 (s, 1H), 7.07 (d, J=7.2 Hz, 1H), 6.38 (s, 1H), 5.60 (s, 2H), 5.39 (s, 1H), 4.99-4.55 (m, 1H), 4.41-4.07 (m, 1H), 3.68 (d, J=6.5 Hz, 1H), 3.42 (d, J=10.5 Hz, 1H), 3.26 (s, 3H), 3.01 (d, J=10.0 Hz, 1H), 2.74-2.67 (m, 1H), 2.38 (s, 3H), 2.12 (d, J=10.3 Hz, 1H), 1.68-1.33 (m, 2H), 1,29 (d, J=6.6 Hz, 3H), 1.11 (d, J=6.3 Hz, 3H), 0.95 (t, J=7.2 Hz, 4H) ppm. MS: We 474 (M+1)⁺.

Compound A173: 2-(7-((2S,5R)-5-ethyl-4-(1-(3-fluoro-2-methylpyrazolo[1,5-a]pyrimidin-5-yl)ethyl)-2-methylpiperazin-1-yl)-4-methyl-5-oxo-4,5-dihydro-2H-pyrazolo[4,3-b]pyridin-2-yl)acetonitrile

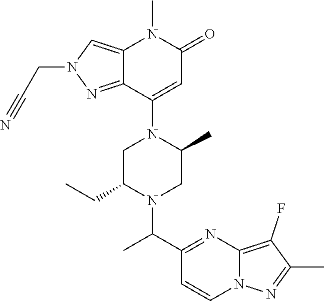 [SMILES: CC[C@@H]1CN(c2cc(=O)n(C)c3cn(CC#N)nc23)[C@@H](C)CN1C(C)c1ccn2nc(C)c(F)c2n1]

To a solution of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (50 mg, 0.15 mmol) in CH₃CN (5 mL) and was added 1-(3-fluoro-2-methylpyrazolo[1,5-a]pyrimidin-5-yl)ethan-1-ol (93 mg, 0.45 mmol), (cyanomethyl)trimethylphosphonium iodide (146 mg, 0.6 mmol) and DIPEA(197 mg, 1.5 mmol).The resulting mixture was stirred at 105° C. overnight. The reaction solvent was removed under reduce pressure. The resulting residue was purified by flash column chromatography to give the titled compound(crude), which was further separated into Compound A173a (2 mg, 2%) and Compound A173b (2 mg, 2%) by Prep-HPLC (Method B).

Compound A173a (the earlier peak): ¹H NMR (400 MHz, DMSO-d₆) δ 8.84 (d, J=7.2 Hz, 1H), 7.97 (s, 1H), 7.07 (d, J=7.3 Hz, 1H), 5.58 (s, 2H), 5.41 (s, 1H), 4.71-4.20 (m, 2H), 3.89 (d, J=6.6 Hz, 1H), 3.26 (s, 3H), 3.22 (s, 1H), 2.91 (d, J=8.3 Hz, 1H), 2.68 (d, J=11.7 Hz, 1H), 2,47-2,42 (m, 1H), 2.39 (s, 3H), 1.52 (d, J=7.4 Hz, 2H), 1.32 (d, J=6.5 Hz, 3H), 1,21 (d, J=6.1 Hz, 3H), 0.73 (t, J=7.1 Hz, 3H) ppm. MS: M/e 492 (M+1)⁺.

Compound A173b (the later peak): ¹H NMR (400 MHz, DMSO-d₆) δ 8.84 (d, J=7.0 Hz, 1H), 7.97 (s, 1H), 7.08 (d, J=7.2 Hz, 1H), 5.60 (s, 2H), 5.39 (s, 1H), 4.97-4.09 (m, 2H), 3.71 (d, J=6.4 Hz, 1H), 3.42 (d, J=12.2 Hz, 1H), 3.26 (s, 3H), 3.01 (d, J=9.6 Hz, 1H), 2.71 (d, J=8.7 Hz, 1H), 2.38 (s, 3H), 2.13 (d, 0.1=11.6 Hz, 1H), 1.71-1.35 (m, 2H), 1.30 (d, J=6.4 Hz, 3H), 1.10 (d, J=6.2 Hz, 3H), 0.95 (t, J=7.0 Hz, 3H) ppm. MS: M/e 492 (M+1)⁺.

### Compound A174: 2-(7-((2S,5R)-4-(1-(3-chloroimidazo[1,2-b]pyridazin-6-yl)ethyl)-2,5-diethylpiperazin-1-yl)-4-methyl-5-oxo-4,5-dihydro-2H-pyrazolo[4,3-d]pyrimidin-2-yl)acetonitrile

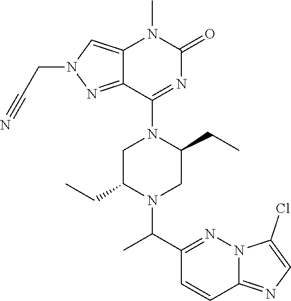 [SMILES: CC[C@H]1CN(C(C)c2ccc3ncc(Cl)n3n2)[C@H](CC)CN1c1nc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-d]pyrimidin-2-yl)acetonitrile (33 mg, 0.1 mmol), 1-(3-chloroimidazo[1,2-b]pyridazin-6-yl)ethan-1-ol (39 mg, 0.2 mmol) and (cyanomethyl)trimethylphosphonium iodide (97 mg, 0.4 mmol) in CH₃CN (2 mL) was added DIPEA (51 mg, 0.4 mmol). The reaction mixture was sealed in a bottle and heated at 100° C. for 16 hours, and then cooled to room temperature, diluted with water(30 mL), extracted with EtOAc (10 mL×2). The combined organic layers were washed with brine, dried over Na₂SO₄, filtered, and concentrated to dryness. The resulting residue was purified and separated into Compound A174a (1,22 mg, 2,4%) and Compound A174b (1.71 mg, 3.4%) by Prep-HPLC (Method A).

Compound A174a (the earlier peak): ¹H NMR (400 MHz, CDCl₃) δ 7.93 (s, 1H), 7.70 (s, 1H), 7.36 (d, J=8.8 Hz, 2H), 6.38-5.92 (m, 1H), 5.29-5.09 (m, 1H), 3.93 (s, 1H), 3.82 (s, 1H), 3.74 (s, 3H), 3.68-3.30 (m, 1H), 3.14 (d, J=20.9 Hz, 1H), 2.90-2.71 (m, 1H), 2.22 (d, J=11.8 Hz, 1H), 1.96 (s, 1H), 1.67 (s, 1H), 1.41 (s, 6H), 1.01 (d, 0.1=22,4 Hz, 3H), 0.73 (dd, J=18.2, 10.7 Hz, 3H) ppm. MS: M/e 509 (M+1)⁺.

Compound A174b (the later peak): ): ¹H NMR (400 MHz, CDCl₃) δ 7.96 (s, 1H), 7.70 (s, 1H), 7.35 (d, J=7.3 Hz, 2H), 6.24-5.97 (m, 1H), 5.40-5.01 (m, 1H), 4.09 (s, 1H), 3.82 (s, 1H), 3.74 (s, 3H), 3.49-3.05(m, 1.5H), 3.00-2.80 (m, 2H), 2,49-2.32 (m, 1.5H), 1.95 (dd, J=26.0, 19.0 Hz, 3H), 1.70-1.50 (m, 1H), 1.43 (d, J=5.3 Hz, 3H), 0.96-0.74 (m, 6H) ppm. MS: M/e 509 (M+1)⁺.

### Compound A175: 2-(7-((2S,5R)-4-(1-(2-chloropyrazolo[1,5-a]pyrimidin-5-yl)ethyl)-2,5-diethylpiperazin-1-yl)-4-methyl-5-oxo-4,5-dihydro-2H-pyrazolo[4,3-b]pyridin-2-yl)acetonitrile and Compound A176: 2-(7-((2S,5R)-4-(1-(2-chloro-3-fluoropyrazolo[1,5-a]pyrimidin-5-yl)ethyl)-2,5-diethylpiperazin-1-yl)-4-methyl-5-oxo-4,5-dihydro-2H-pyrazolo[4,3-b]pyridin-2-yl)acetonitrile

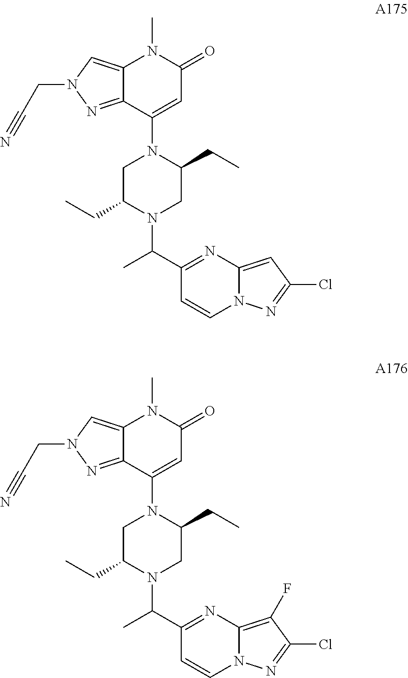 [SMILES: CC[C@H]1CN(C(C)c2ccn3nc(Cl)cc3n2)[C@H](CC)CN1c1cc(=O)n(C)c2cn(CC#N)nc12 ; CC[C@H]1CN(C(C)c2ccn3nc(Cl)c(F)c3n2)[C@H](CC)CN1c1cc(=O)n(C)c2cn(CC#N)nc12]

### Step A: 5-bromo-2-chloro-3-fluoropyrazolo[1,5-a]pyrimidine

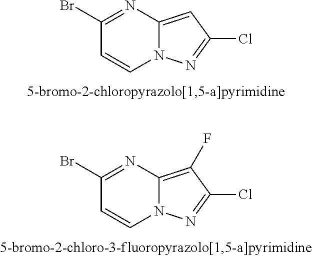 [SMILES: Clc1cc2nc(Br)ccn2n1 ; Fc1c(Cl)nn2ccc(Br)nc12]

To a solution of 5-bromo-2-chloropyrazolo[1,5-a]pyrimidine (800 mg, 3.46 mmol) in MeCN (20 mL) was added Select F (1.8 g, 5.2 mmol). The reaction mixture was stirred at RT for 16 hrs. The mixture was cooled down to RT and concentrated in vacuo. The resulting residue was purified by flash column chromatography with 0-100% EtOAc in PE to give a mixture of 5-bromo-2-chloropyrazolo[1,5-a]pyrimidine and 5-bromo-2-chloro-3-fluoropyrazolo[1,5-a]pyrimidine (200 mg). MS: M/e 250 (M+1)⁺.

### Step B: 1-(2-chloro-3-fluoropyrazolo[1,5-a]pyrimidin-5-yl)ethan-1-one

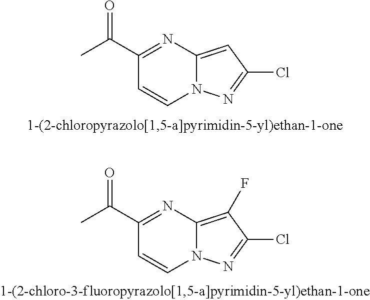

To a solution of 5-bromo-2-chloropyrazolo[1,5-a]pyrimidine and 5-bromo-2-chloro-3-fluoropyrazolo[1,5-a]pyrimidine from last step (200 mg, 0.8 mmol) in toluene was added Pd(PPh₃)₂Cl₂ (70 mg, 0.1 mmol) and tributyl(1-ethoxyvinyl)stannane (365 mg, 1 mmol). The reaction mixture was stirred at 100° C. under N₂ for 8 hours. The reaction mixture was added 4N HCl/Dioxane (1 mL) and stirred at RT for 15 mins. The mixture was added saturated NaHCO₃ aqueous solution (30 mL) and then extracted with EtOAc (30 mL×3). The organic layer was washed with brine (30 mL), dried and concentrated. The resulting residue was purified by flash column chromatography with 0-100% EA in PE to give a mixture of 1-(2-chloropyrazolo[1,5-a]pyrimidin-5-yl)ethan-1-one and 1-(2-chloro-3-fluoropyrazolo[1,5-a]pyrimidin-5-yl)ethan-1-one (140 mg). MS: M/e 214 (M+1)⁺.

### Step C: 1-(2-chloro-3-fluoropyrazolo[1,5-a]pyrimidin-5-yl)ethan-1-ol

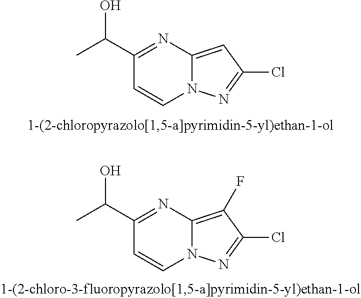

To a solution of 1-(2-chloropyrazolo[1,5-a]pyrimidin-5-yl)ethan-1-one and 1-(2-chloro-3-fluoropyrazolo[1,5-a]pyrimidin-5-yl)ethan-1-one (140 mg, 1.1 mmol) in MeOH was added NaBH₄ (38 mg, 1 mmol) and the reaction mixture was stirred at RT for 15 mins. The reaction mixture was added H₂O (30 mL) and then extracted with DCM (30 mL×3). The organic layer was washed with brine (30 mL), dried and concentrated. The resulting residue was purified by flash column chromatography with 0-100% EA in PE to give a mixture of 1-(2-chloropyrazolo[1,5-a]pyrimidin-5-yl)ethan-1-ol and 1-(2-chloro-3-fluoropyrazolo[1,5-a]pyrimidin-5-yl)ethan-1-ol (100 mg, 75%). MS: M/e:216 (M+1)⁺.

### Step D: 2-(7-((2S,5R′-4-(1-(2-chloropyrazolo[1,5-a]pyrimidin-5-yl)ethyl)-2,5-diethylpiperazin-1-yl)-4-methyl-5-oxo-4,5-dihydro-2H-pyrazolo[4,3-b]pyridin-2-yl)acetonitrile and 2-(7-((2S,5R)-4-(1-(2-chloro-3-fluoropyrazolo[1,5-al pyrimidin-5-yl)ethyl)-2,5-diethylpiperazin-1-yl-1-4-methyl-5-oxo-4,5-dihydro-2H-pyrazolo[4,3-b]pyridin-2-yl)acetonitrile

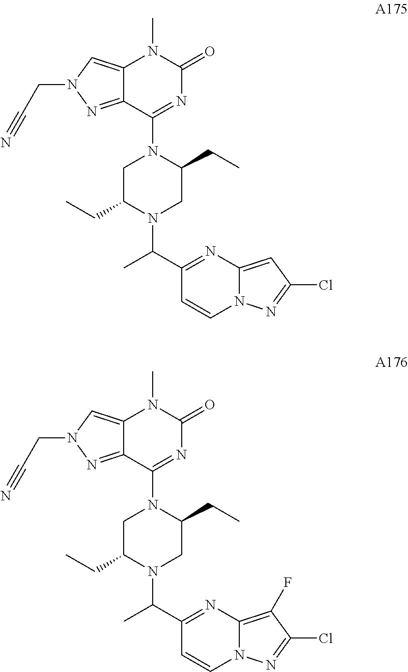 [SMILES: CC[C@H]1CN(C(C)c2ccn3nc(Cl)cc3n2)[C@H](CC)CN1c1nc(=O)n(C)c2cn(CC#N)nc12 ; CC[C@H]1CN(C(C)c2ccn3nc(Cl)c(F)c3n2)[C@H](CC)CN1c1nc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (30 mg, 0.1 mmol), the mixture of 1-(2-chloropyrazolo[1,5-a]pyrimidin-5-yl)ethan-1-ol and 1-(2-chloro-3-fluoropyrazolo[1,5-a]pyrimidin-5-yl)ethan-1-ol (30 mg, 0.12 mmol), (cyanomethyl)trimethylphosphonium iodide (120 mg, 0.5 mmol) and DIPEA (129 mg, 1 mmol) in MeCN (3 mL) was stirred at 100° C. for 16 hours. The resulted mixture was diluted with EtOAc (10 mL), washed with brine (5 mL×3), dried and concentrated. The resulting residue was purified by flash column chromatography to give the titled compound, which was further separated into Compound A175 (3 mg, 12%) and Compound A176 (2 mg, 8%) by Prep-HPLC (Method A).

Compound A175 (the earlier peak): ¹H NMR (400 MHz, CD₃OD) δ 8.80-8.78 (m, 1H), 7.92 (s, 1H), 7.30 (m, 1H), 6.58 (d, J=4.3 Hz, 1H), 5.57 (s, 1H), 5.47 (d, J=4.1 Hz, 2H), 3.88-3.86 (m, 1H), 3.54-3.52 (m, 1H), 3.44-3.42 (s, 3H), 3.40-3.38 (m, 1H), 3.13-3.11 (m, 1H), 2.98-2.96 (m, 1H), 2.83-2.81 (m, 1H), 2.38-2.36 (m, 1H), 2.08-2.06 (m, 1H), 1.82-1.80 (m, 1H), 1.62-1.60 (m, 2H), 1.41-1.39 (m, 3H), 1.03 (d, J=7.0 Hz, 1.5H), 0.93 (d, J=7.0 Hz, 1.5H), 0.77 (d, J=7.2 Hz, 1.5H), 0.72 (d, J=7.2 Hz, 1.5H) ppm. MS: M/e 508 (M+1)⁺.

Compound A176 (the later peak): ¹H NMR (400 MHz, CD₃OD) δ 8.69 (d, J=6.8 Hz, 1H), 7.93 (s, 1H), 7.28-7.26 (m, 1H), 5.57 (s, 1H), 5.47 (d, J=3.7 Hz, 2H), 3.94-3.92 (m, 1H), 3.50-3.48 (m, 4H), 3.38-3.36 (m, 1H), 3.13-3.11 (m, 1H), 2.98-2.96 (m, 1H), 2.83-2.81 (m, 1H), 2,50-2,48 (m, 1H), 2.06-2.04 (m, 1H), 1.84-1.82 (m, 2H), 1.65-1.63 (m, 1H), 1.42-1.40 (m, 3H), 1.02 (t, .=7.1 Hz, 1.5H), 0.91 (t, J=7.0 Hz, 1.5H), 0.80 (d, J=7.5 Hz, 1.5H), 0.71 (d, J=7.5 Hz, 1.5H) ppm. MS: M/e 526 (M+1)⁺.

### Compound A177: 2-(7-((2S,5R)-4-(1-(2-chloropyrazolo[1,5-a]pyrimidin-5-yl)ethyl)-5-ethyl-2-methylpiperazin-1-yl)-4-methyl-5-oxo-4,5-dihydro-2H-pyrazolo[4,3-b]pyridin-2-yl)acetonitrile and Compound A178: 2-(7-((2S,5R)-4-(1-(2-chloro-3-fluoropyrazolo[1,5-a] pyrimidin-5-yl)ethyl)-5-ethyl-2-methylpiperazin-1-yl)-4-methyl-5-oxo-4,5-dihydro-2H-pyrazolo[4,3-b]pyridin-2-yl)acetonitrile

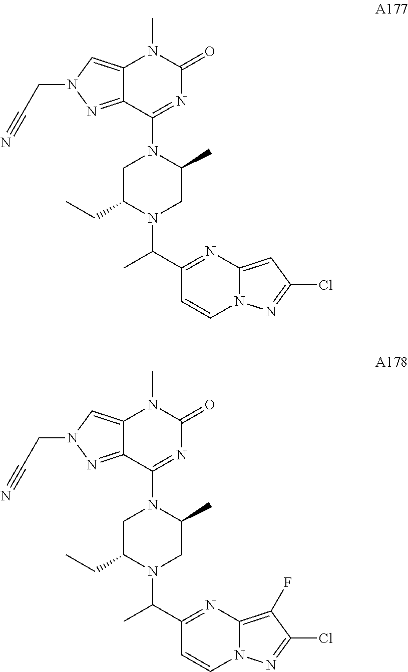 [SMILES: CC[C@@H]1CN(c2nc(=O)n(C)c3cn(CC#N)nc23)[C@@H](C)CN1C(C)c1ccn2nc(Cl)cc2n1 ; CC[C@@H]1CN(c2nc(=O)n(C)c3cn(CC#N)nc23)[C@@H](C)CN1C(C)c1ccn2nc(Cl)c(F)c2n1]

A mixture of 2-(7-((2S,5R)-5-ethyl-2-methylpiperazin-1-yl)-4-methyl-5-oxo-4,5-dihydro-2H-pyrazolo[4,3-b]pyridin-2-yl)acetonitrile (30 mg, 0.1 mmol), the mixture of 1-(2-chloropyrazolo[1,5-a]pyrimidin-5-yl)ethan-1-ol and 1-(2-chloro-3-fluoropyrazolo[1,5-a]pyrimidin-5-yl)ethan-1-ol(30 mg, 0.12 mmol), (cyanomethyl)trimethylphosphonium iodide (120 mg, 0.5 mmol) and DIPEA (129 mg, 1 mmol) in MeCN (3 mL) was stirred at 100° C. for 16 hours. The resulted mixture was diluted with EtOAc (10 mL), washed with brine (5 mL×3), dried and concentrated. The resulting residue was purified by flash column chromatography to give the titled compound, which was further separated into Compound A177a (0.95 mg, 3.8%), Compound A177b (1.1 mg, 4.3%), Compound A178a (1.1 mg, 4.5%) and Compound A178b (0.98 mg, 3.9%) by Prep-HPLC (Method A).

Compound A177a (the first peak): ¹H NMR (400 MHz, CD₃OD) δ 8.78 (d, J=7.3 Hz, 1H), 7.92 (s, 1H), 7.29 (d, J=7.2 Hz, 1H), 6.59 (s, 1H), 5.59 (s, 1H), 5.45 (s, 2H), 4.70 (s, 1H), 3.97 (d, J=6.6 Hz, 1H), 3.42-3.40 (m, 5H), 3.05-3.03 (m, 1H), 2.85-2.83 (m 1H), 2,51-2,49 (m, 1H), 1.63-1.61 (m, 2H), 1.40 (t, J=6.7 Hz, 6H), 0.82 (t, J=7.3 Hz, 3H) ppm. MS: M/e 494 (M+1)⁺.

Compound A177b (the second peak): ¹H NMR (400 MHz, CD₃OD) δ 8.77 (d, J=7.3 Hz, 1H), 7.93 (s, 1H), 7.34 (d, J=7.2 Hz, 1H), 6.57 (s, 1H), 5.58 (s, 1H), 5.47 (s, 2H), 5.02-5.00 (m, 1H), 4.45-4.43 (m, 1H), 3.80-3.78 (m, 1H), 3.58-3.56 (m, 1H), 3.45-3.43 (s, 3H), 3.13-3.11 (m, 1H), 2.87-2.85 (m, 1H), 2.25-2.23 (m, 1H), 1.72-1.70 (m, 1H), 1.55-1.53 (m, 1H), 1.41 (d, J=6.7 Hz, 3H), 1,26 (d, J=6.4 Hz, 3H), 1.06 (d, J=7.2 Hz, 3H) ppm. MS: M/e 494 (M+1)⁺.

Compound A178a (the third peak): ¹H NMR (400 MHz, CD₃OD) δ 8.69 (d, J=7.3 Hz, 1H), 7.92 (s, 1H), 7.28 (d, J=7.4 Hz, 1H), 5.60 (s, 1H), 5.45 (s, 2H), 4.71-4.69 (m, 2H), 4.00-3.98 (m, 1H), 3.45-3.43 (m, 4H), 3.06-3.04 (m, 1H), 2.84-2.82 (m, 1H), 2,53-2,51 (m, 1H), 1.65-1.63 (m, 2H), 1.40 (dd, J=13.1, 6.6 Hz, 6H), 0.83 (d, J=7.3 Hz, 3H) ppm. MS: M/e 512 (M+1)⁺.

Compound A178b (the fourth peak): ¹H NMR (400 MHz, CD₃OD) δ 8.68 (d, J=7.2 Hz, 1H), 7.93 (s, 1H), 7.33 (d, J=7.3 Hz, 1H), 5.58 (s, 1H), 5.47 (s, 2H), 5.03 (s, 1H), 4.44 (s, 1H), 3.83-3.81 (m, 1H), 3.59-3.57 (m, 1H), 3.46 (s, 3H), 3.13-3.11 (m, 1H), 2.87-2.85 (m, 1H), 2.27-2.25 (m, 1H), 1.72-1.70 (m, 1H), 1.56-1.54 (m, 1H), 1.41 (d, J=6.6 Hz, 3H), 1,26 (d, J=6.5 Hz, 3H), 1.06 (t, J=7.3 Hz, 3H) ppm. MS: M/e 512 (M+1)⁺.

### Compound A179: 2-(7-((2S,5R)-4-(1-(7-fluoro-3,3-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile

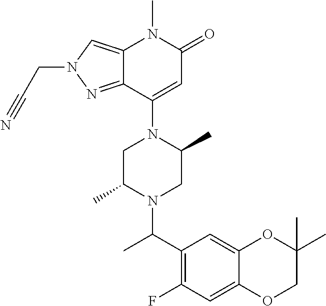 [SMILES: CC(c1cc2c(cc1F)OCC(C)(C)O2)N1C[C@H](C)N(c2cc(=O)n(C)c3cn(CC#N)nc23)C[C@H]1C]

### Step A: 2,4-difluoro-5-hydroxybenzonitrile

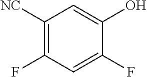 [SMILES: N#Cc1cc(O)c(F)cc1F]

To a solution of 5-bromo-2,4-difluorophenol (1.0 g, 4.785 mmol), t-BuXphos-Pd-G3 (190 mg, 0.239 mmol), t-BuXphos (204 mg, 0.48 mmol) and Zn(CN)₂ (1.12 g, 9.570 mmol) in THF (20 mL) and H₂O (10 mL) was degassed 3 times under N₂ atmosphere. Then the mixture was stirred at 70° C. for 12 hours. After filtration, the reaction mixture was diluted with water and extracted with EA (50 mL×2). The combined organic layers were washed with brine, dried over Na₂SO₄, filtered and concentrated to dryness. The resulting residue was purified by flash column chromatography (PE:EA=5:1) to give the titled compound (730 mg, 98%).

### Step B: 7-fluoro-3.3-dimethyl-2.3-dihydrobenzo[b][1,4]dioxine-6-carbonitrile

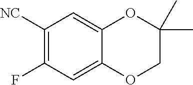

To a solution of 2,4-difluoro-5-hydroxybenzonitrile (700 mg, 4.516 mmol) and Cs₂CO₃ (5.15 g, 13.548 mmol) in DMAc (25 mL) was added 2,2-dimethyloxirane (4.885 g, 67.74 mmol). The reaction was stirred at 90° C. for 12 hours. After filtration, the reaction mixture was diluted with water, extracted with PE (60 mL×3), washed with brine, dried over Na₂SO₄, filtered, and concentrated to dryness. The resulting residue was purified by flash column chromatography (PE:EA=5:1) to give the titled compound (220 mg, 24%). ¹H NMR (400 MHz, CDCl₃) δ 7.09 (d, J=6.1 Hz, 1H), 6.66 (d, J=9.7 Hz, 1H), 3.88 (s, 2H), 1.36 (s, 6H).

### Step C: 7-fluoro-3.3-dimethyl-2.3-dihydrobenzo[b][1,4]dioxine-6carbaldehyde

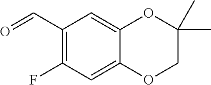

To a solution of 7-fluoro-3,3-dimethyl-2,3-dihydrobenzo[b][1,4]dioxine-6-carbaldehyde (200 mg, 0.966 mmol) in THF (15 mL) was degassed 3 times under N₂ atmosphere. Then added DIBAL-H (1.45 mL, 1M, 1.45 mmol) dropwise at 0° C. The mixture was stirred at 0° C. for 4 hours. The reaction solution was quenched by 1M HCl. After filtration, the reaction mixture was diluted with water, extracted with EA (50 mL×3), washed with brine, dried over Na₂SO₄, filtered, and concentrated to dryness. The resulting residue was purified by flash column chromatography (PE:EA=5:1) to give the titled compound (120 mg, 54%).

### Step D: 1-(7-fluoro-3.3-dimethyl-2.3-dihydrobenzo[b][1,4]dioxin-6-yl)ethan-1-ol

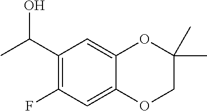 [SMILES: CC(O)c1cc2c(cc1F)OCC(C)(C)O2]

To a solution of 7-fluoro-3,3-dimethyl-2,3-dihydrobenzo[b][1,4]dioxine-6-carbaldehyde (110 mg, 0.523 mmol) in THF (10 mL) was degassed 3 times under N₂ atmosphere. Then added CH₃MgBr (0.35 mL, 3M, 1.048 mmol) dropwise at 0° C. The mixture was stirred at 0° C. for 4 hours. The reaction solution was quenched with saturated NH₄Cl, extracted with EA (50 mL×2), washed with brine, dried over Na₂SO₄, filtered, and concentrated to dryness. The resulting residue was purified by flash column chromatography (PE:EA=5:1) to give the titled compound (86 mg, 73%).

### Step E: 2-(7-((2S,5R)-4-(1-(7-fluoro-3.3-dimethyl-2.3-dihydrobenzo[b]1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile

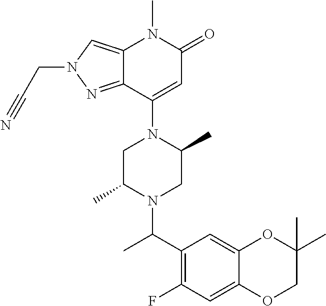 [SMILES: CC(c1cc2c(cc1F)OCC(C)(C)O2)N1C[C@H](C)N(c2cc(=O)n(C)c3cn(CC#N)nc23)C[C@H]1C]

A solution of 2-(7-((2S,5R)-2,5-dimethylpiperazin-1-yl)-4-methyl-5-oxo-4,5-dihydro-2H-pyrazolo[4,3-b]pyridin-2-yl)acetonitrile (120 mg, 0.40 mmol), 1-(7-fluoro-3,3-dimethyl-2,3-dihydrobenzo[b][1,4]dioxin-6-yl)ethan-1-ol (90 mg, 0.40 mmol), (cyanomethyl)trimethylphosphonium iodide (290 mg, 1.19 mmol) and DIPEA (514 mg, 3.98 mmol) in CH₃CN (3 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A179 (15 mg), which was further separated into Compound A179a (3 mg) and Compound A179b (4 mg) by Prep-HPLC (Method A).

Compound A179a (the earlier peak): ¹H NMR (DMSO-d₆) δ 8.35 (s, 0.28H), 7.99 (s, 1H), 7.01 (d, J=7.0 Hz, 1H), 6.65 (d, J=11.0 Hz, 1H), 5.62 (s, 2H), 5.41 (s, 1H), 4.55 (s, 1H), 4.31 (s, 1H), 3.91 (s, 2H), 3.71 (q, J=6.5 Hz, 1H), 3.44 (d, J=9.5 Hz, 2H), 3.28 (s, 3H), 2.66 (d, J=11.8 Hz, 1H), 2.15 (d, J=11.7 Hz, 1H), 1,29-1,23 (m, 9H), 1.10 (d, J=6.4 Hz, 3H), 1.04 (d, J=6.3 Hz, 3H) ppm. MS: M/e 509 (M+1)⁺.

Compound A179b (the later peak): ¹H NMR (400 MHz, DMSO-d₆) δ 8.31 (s, 0.35H), 7.99 (s, 1H), 6.97 (d, J=6.9 Hz, 1H), 6.68 (d, J=11.0 Hz, 1H), 5.63 (s, 2H), 5.40 (s, 1H), 4.84 (s, 1H), 3.93 (s, 2H), 3.85 (d, J=6.3 Hz, 1H), 3.28-3.20 (m, 5H), 2.91-2.82 (m, 2H), 2.71 (d, J=11.1 Hz, 1H), 1,29 (d, J=4.5 Hz, 6H), 1,23 (d, J=6.4 Hz, 6H), 0.91 (d, J=6.5 Hz, 3H) ppm. MS: M/e 509 (M+1)⁺.

### Compound A180: 2-(7-((2S,5R)-2,5-diethyl-4-(1-(imidazo[1,2-b]pyridazin-2-yl)ethyl)piperazin-1-yl)-4-methyl-5-oxo-4,5-dihydro-2H-pyrazolo[4,3-b]pyridin-2-yl)acetonitrile

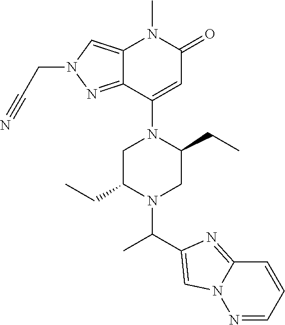 [SMILES: CC[C@H]1CN(C(C)c2cn3ncccc3n2)[C@H](CC)CN1c1cc(=O)n(C)c2cn(CC#N)nc12]

### Step A: 1-(imidazo[1,2-b]pyridazin-2-yl)ethan-1-ol

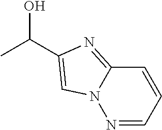

To a stirred solution of imidazo[1,2-b]pyridazine-2-carbaldehyde (200 mg, 1.36 mmol) in THF (10 mL) was added dropwise MeMgBr (1.0 M, 1.4 mL, 1.36 mmol) at 0° C. After stirred for 30 min, the reaction was quenched with aq·NH₄Cl, extracted with EtOAc (30 mL×3). The combined organic layers were washed with brine, dried over Na₂SO₄, concentrated and purified by flash column chromatography to give the titled compound (130 mg, 59%) as a colorless oil. MS: M/e 163 (M+1)⁺.

### Step B: 2-(7-((2S,5R)-2,5-diethyl-4-(1-(imidazo[1,2-b]pyridazin-2-yl)ethyl)-4-methyl-5-oxo-4,5-dihydro-2H-pyrazolo[4,3-b]pyridin-2-yl)acetonitrile

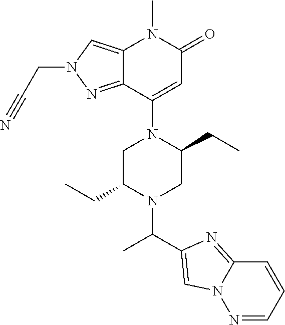 [SMILES: CC[C@H]1CN(C(C)c2cn3ncccc3n2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(imidazo[1,2-a]pyridin-2-yl)ethan-1-ol (27 mg, 0.17 mmol), (cyanomethyl)trimethylphosphonium iodide (111 mg, 0.46 mmol) and DIPEA (98 mg, 0.76 mmol). The resulting mixture was stirred at 105° C. overnight. The reaction solvent was removed under reduce pressure to afford crude product. The resulting residue was purified Prep-HPLC (Method A) to give the titled compound (26 mg, 36%). ¹H NMR (400 MHz, CD₃OD) δ 8.42 (s, 1H), 8.09 (d, J=20.8 Hz, 1H), 8.01-7.91 (m, 2H), 7.25-7.18 (m, 1H), 5.55 (d, J=4.9 Hz, 1H), 5.47 (d, J=3.4 Hz, 2H), 4.17-3.97 (m, 1H), 3.56-3.45 (m, 1H), 3.43 (s, 3H), 3.24-3.04 (m, 2H), 2.90-2,57 (m, 3H), 2.02-1.60 (m, 4H), 1.51 (dd, J=26.1, 6.8 Hz, 3H), 0.97 (dd, J=22.0, 7.6 Hz, 3H), 0.76 (dt, J=39.8, 7.2 Hz, 3H). MS: M/e 474 (M+1)+

### Compound A181: 2-(7-((2S,5R)-2,5-diethyl-4-(1-(thiazolo[5,4-b]pyridin-2-yl)ethyl)piperazin-1-yl)-4-methyl-5-oxo-4,5-dihydro-2H-pyrazolo[4,3-b]pyridin-2-yl)acetonitrile

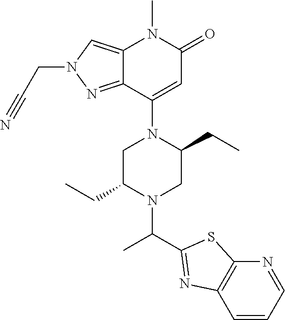 [SMILES: CC[C@H]1CN(C(C)c2nc3cccnc3s2)[C@H](CC)CN1c1cc(=O)n(C)c2cn(CC#N)nc12]

### Step A: thiazolo[5.4-b]pyridin-2-amine

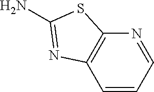

To a solution of 2-chloropyridin-3-amine (9.0 g, 70.3 mmol) in HCl (conc., 90 mL) was added potassium thiocyanate (10.2 g, 105.1 mmol) at room temperature and the resulted mixture was stirred at 100° C. for 4 hours. The reaction mixture was cooled to room temperature and stirred for 16 hours. The mixture was concentrated to dryness and the residue was basified with saturated solution of NaHCO₃ to pH-9. The resulting suspension was filtered and the filter cake was washed with water (100 mL), dried under high vacuum to obtain the titled compound (7.9 g).

¹H NMR (400 MHz, DMSO-d6) δ 8.09 (d, J=4.0 Hz, 1H), 7.79 (s, 2H), 7.61 (d, J=8.0 Hz, 1H), 7.30-7.18 (m, 1H). MS: M/e 152 (M+1)⁺.

### Step B: 2-bromo thiazolo[5.4-b]pyridine

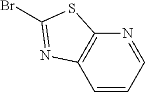

To a 0° C. solution of thiazolo[5,4-b]pyridin-2-amine (2.1 g, 13.9 mmol) in MeCN (40 mL) was added isopentyl nitrite (3.3 g, 27.8 mmol) and followed by CuBr₂ (6.2 g, 27.8 mmol) in portions. The resulted mixture was stirred at room temperature for 16 hours. The mixture was diluted with EtOAc (50 mL), filtered through a celite pad and the filtrate was washed with brine (50 mL×3), dried over Na₂SO₄, filtered and concentrated to dryness. The resulted residue was purified by column chromatography to give the titled compound (900 mg, yield: 30%). ¹H NMR (400 MHz, DMSO-d6) δ 8.66 (d, J=4.8 Hz, 1H), 8.44 (dd, J=8.0, 1,2 Hz, 1H), 7.63 (dd, J=8.0, 4.4 Hz, 1H).

### Step C: 1-(thiazolo[5.4-b]pyridin-2-yl)ethan-1-ol

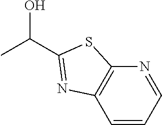

To a −70° C. solution of 2-bromothiazolo[5,4-b]pyridine (215 mg, 1.0 mmol) in THF (4 mL) was added n-BuLi (1.6 M, 0.75 mL, 1,2 mmol) in drops under N₂. After stirring for 10 minutes at −70° C., a solution of acetaldehyde (90 mg, 2.0 mmol) in THF (1 mL) was added. Then the mixture was allowed warm to room temperature and stirred for 30 minutes. Aqueous solution of NH₄Cl (20 mL) was added and the mixture was extracted with EtOAc (10 mL×3), the organics were combined and washed with brine (10 mL×2), dried over Na₂SO₄, filtered and concentrated to dryness. The resulted residue was purified by column chromatography to give the titled compound (62 mg, yield: 34%). ¹H NMR (400 MHz, DMSO-d6) δ 8.59 (dd, J=4.8, 1,2 Hz, 1H), 8.33 (dd, J=8.0, 1,2 Hz, 1H), 7.56 (dd, J=8.4, 4.8 Hz, 1H), 6.46 (d, J=5.2 Hz, 1H), 5.12-5.01 (m, 1H), 1.55 (d, J=6.4 Hz, 3H). MS: M/e 181 (M+1)⁺.

### Step D: 2-(7-((2S,5R)-2,5-diethyl-4-(1-(thiazolo[5.4-b]pyridin-2-yl)ethyl)piperazin-1-yl)-4-methyl-5-oxo-4,5-dihydro-2H-pyrazolo[4,3-b]pyridin-2-yl)acetonitrile

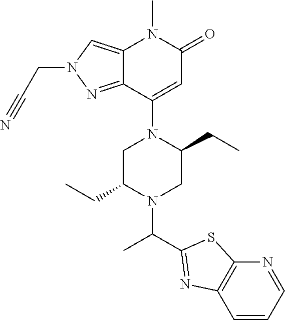 [SMILES: CC[C@H]1CN(C(C)c2nc3cccnc3s2)[C@H](CC)CN1c1cc(=O)n(C)c2cn(CC#N)nc12]

To a mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (44 mg, 0.134 mmol), 1-(thiazolo[5,4-b]pyridin-2-yl)ethan-1-ol (48 mg, 0.266 mmol) and (cyanomethyl)trimethylphosphonium iodide (97 mg, 0.400 mmol) in CH₃CN (1 mL) was added DIPEA (86 mg, 0.660 mmol). The mixture was stirred at 100° C. in a sealed tube for 16 hours. The mixture was cooled and diluted with EtOAc (20 mL), washed with brine (10 mL×3), dried over Na₂SO₄, filtered and concentrated under reduced pressure. The resulting residue was purified by flash column chromatography and Prep-HPLC (Method A) to give the titled compound (15 mg, 23%). ¹H NMR (400 MHz, CD₃OD) δ 8.60-8.50 (m, 1H), 8.31-8.21 (m, 1H), 7.95 (s, 1H), 7.58-7.50 (m, 1H), 5.70-5.53 (m, 1H), 5.53-5.41 (m, 2H), 5.35-4.89 (m, 1H), 4.53-4.02 (m, 2H), 3.68-3.39 (m, 4H), 3.21-3.05 (m, 1H), 2.99-2.86 (m, 1H), 2.86-2.62 (m, 1H), 2.22-1.46 (m, 7H), 1.09-0.75 (m, 6H). MS: M/e 491 (M+1)⁺.

### Compound A182: 2-(7-((2S,5R)-4-(1-(5-((4,4-difluoropiperidin-1-yl)methyl)pyridin-2-yl)ethyl)-2,5-diethylpiperazin-1-yl)-4-methyl-5-oxo-4,5-dihydro-2H-pyrazolo[4,3-b]pyridin-2-yl)acetonitrile

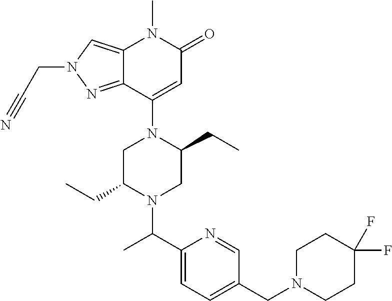 [SMILES: CC[C@H]1CN(C(C)c2ccc(CN3CCC(F)(F)CC3)cn2)[C@H](CC)CN1c1cc(=O)n(C)c2cn(CC#N)nc12]

### Step A: methyl 5-((4.4-difluoropiperidin-1-yl)methyl)picolinate

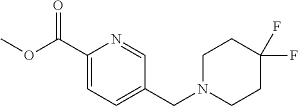 [SMILES: COC(=O)c1ccc(CN2CCC(F)(F)CC2)cn1]

A solution of methyl 5-formylpicolinate (825 mg, 5 mmol), 4,4-difluoropiperidine (666 mg, 5.5 mmol) and AcOH (0.5 mL) in CH₂Cl₂ (15 mL) was stirred for 2 hours, then NaBH(OAc)₃ (2.12 g, 10 mmol) was added. After then, the mixture was stirred overnight. The mixture was diluted with CH₂Cl₂ (20 mL), then washed with H₂O, brine, dried over Na₂SO₄ and concentrated to dryness. The resulting residue was purified by flash column chromatography to give the titled compound (1.1 g, 81.4%). MS: M/e 271 (M+1)⁺.

### Step B: 5-((4.4-difluoropiperidin-1-yl)methyl)picolinic acid

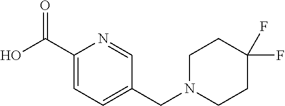 [SMILES: O=C(O)c1ccc(CN2CCC(F)(F)CC2)cn1]

To a stirred solution of methyl 5-((4,4-difluoropiperidin-1-yl)methyl)picolinate (1.1 g, 4 mmol) in MeOH (10 mL) was added aq. NaOH (2.0 M, 4 mL). After then, the mixture was stirred for 4 hours. The mixture was concentrated to give the aqueous layer, which was acidified to pH=3-4 with aq·HCl, then filtered. The cake was collected, dried to give the titled compound (678 mg, 66.2%). MS: M/e 257 (M+1)⁺.

### Step C: 5-((4.4-difluoropiperidine-1-yl)methyl)-N-methoxy-N-methylpicolinamide

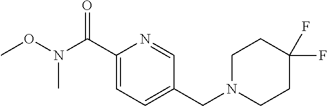 [SMILES: CON(C)C(=O)c1ccc(CN2CCC(F)(F)CC2)cn1]

A mixture of 5-((4,4-difluoropiperidin-1-yl)methyl)picolinic acid (678 mg, 2.65 mmol), N,O-dimethylhydroxylamine hydrochloride (310 mg, 3.18 mmol), HATU (1,2 g, 3.18 mmol) and DIEPA (684 mg, 5.3 mmol) in CH₂Cl₂ (10 mL) was a stirred overnight. The reaction mixture was washed with brine, dried over Na₂SO₄ and concentrated to dryness. The resulting residue was purified by flash column chromatography to give the titled compound (723 mg, 91,2%). MS: M/e 300 (M+1)⁺.

### Step D: 1-(5-((4.4-difluoropiperidin-1-yl)methyl)pyridin-2-yl)ethan-1-one

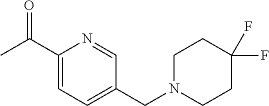 [SMILES: CC(=O)c1ccc(CN2CCC(F)(F)CC2)cn1]

To a stirred solution of 5-((4,4-difluoropiperidin-1-yl)methyl)-N-methoxy-N-methylpicolinamide (723 mg, 2,42 mmol) in THF (10 mL) was added dropwise MeMgBr (3.0 M, 0.97 mL, 2.9 mmol) at 0° C. After then, the mixture was stirred for half an hour. The reaction was quenched with aq·NH₄Cl, extracted with EtOAc (15 mL×3). The combined organic layers were washed with brine, dried over Na₂SO₄ and concentrated to dryness. The resulting residue was purified by flash column chromatography to give the titled compound (375 mg, 61%).

### Step E: 1-(5-((4.4-difluoropiperidin-1-yl)methyl)pyridin-2-yl)ethan-1-ol

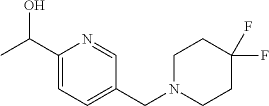 [SMILES: CC(O)c1ccc(CN2CCC(F)(F)CC2)cn1]

To a stirred solution of 1-(5-((4,4-difluoropiperidin-1-yl)methyl)pyridin-2-yl)ethan-1-one (375 mg, 1.48 mmol) in MeOH (5 mL) was added NaBH₄ (56 mg, 1.48 mmol). After then, the mixture was stirred for 10 min. The reaction mixture was poured into H₂O (10 mL) and extracted with EtOAc (10 mL×3). The combined organic layers were washed with brine, dried over Na₂SO₄ and concentrated to give the titled compound (350 mg, 92,4%).

### Step F: 2-(7-((2S,5R)-4-(1-(5-((4.4-difluoropiperidine-1-yl)methyl)pyridin-2-yl)ethyl)-2,5-diethylpiperazin-1-yl)-4-methyl-5-oxo-4,5-dihydro-2H-pyrazolo[4,3-b]pyridin-2-yl)acetonitrile

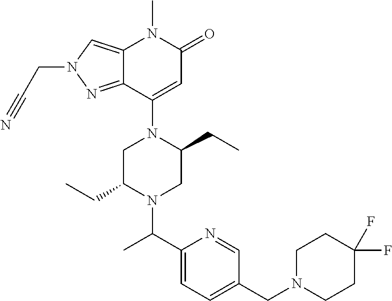 [SMILES: CC[C@H]1CN(C(C)c2ccc(CN3CCC(F)(F)CC3)cn2)[C@H](CC)CN1c1cc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), 1-(5-((4,4-difluoropiperidin-1-yl)methyl)pyridin-2-yl)ethan-1-ol (38.4 mg, 0.15 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄, concentrated to give the residue, which was further purified by Prep-HPLC (Method A) to give the titled compound (12 mg). ¹H NMR (400 MHz, CD₃OD) δ 8.46 (s, 1H), 8.23 (s, 1H), 7.93 (d, J=4.4 Hz, 1H), 7.86 (d, J=8.0 Hz, 1H), 7.66-7.57 (m, 1H), 5.57 (s, 1H), 5.47 (d, J=4.4 Hz, 2H), 4.06-3.88 (m, 1H), 3.65 (s, 2H), 3.61-3.45 (m, 1H), 3.43 (s, 3H), 3.40-3.33 (m, 1H), 3.27-3.20 (m, 1H), 3.08-2.83 (m, 2H), 2.65-2,54 (m, 4H), 2,47-2.33 (m, 1H), 2.07-1.53 (m, 8H), 1.40 (d, J=6.4 Hz, 3H), 1.00 (dt, J=37.2, 7.2 Hz, 3H), 0.69 (dt, J=24, 7.2 Hz, 3H) ppm. MS: M/e 567 (M+1)⁺.

### Compound A183: 2-(7-((2S,5R)-2,5-diethyl-4-(1-(4-(oxetan-3-ylmethyl)phenyl)ethyl)piperazin-1-yl)-4-methyl-5-oxo-4,5-dihydro-2H-pyrazolo[4,3-b]pyridin-2-yl)acetonitrile

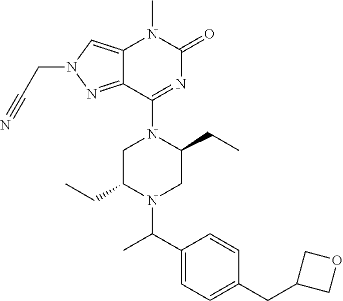 [SMILES: CC[C@H]1CN(C(C)c2ccc(CC3COC3)cc2)[C@H](CC)CN1c1nc(=O)n(C)c2cn(CC#N)nc12]

### Step A: 3-(4-bromobenzylidene)oxetane

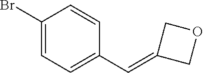 [SMILES: Brc1ccc(C=C2COC2)cc1]

To a solution of (4-bromobenzyl)triphenylphosphonium bromide (1.02 g, 2 mmol) in THF (10 mL) was added a solution of tBuOK in THF (2 mL, 1 mol/L, 2 mmol) drop wise at 0° C. Then a solution of oxetan-3-one (173 mg, 2,4 mmol) in THF (1 mL) was added. The reaction mixture was stirred at 25° C. under N₂ overnight. The mixture was diluted with MTBE (20 mL), filtered, and the filtrate was concentrated to dryness. The residue was purified by flash column chromatography to give the titled compound (220 mg, 50%). ¹H NMR (400 MHz, CDCl₃) δ 7.44 (d, J=8.0 Hz, 2H), 6.86 (d, J=8.4 Hz, 2H), 6.08-6.01 (m, 1H), 5.56-5.48 (m, 2H), 5.39-5.33 (m, 2H) ppm.

### Step B: 1-(4-(oxetan-3-ylidenemethyl)phenyl)ethan-1-one

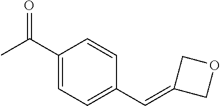

To a solution of 3-(4-bromobenzylidene)oxetane (220 mg, 1 mmol) in toluene (5 mL) was added tributyl(1-ethoxyvinyl)stannane (540 mg, 1.5 mmol) and Pd(PPh₂)₂Cl₂ (70 mg, 0.1 mmol). The reaction mixture was protected by N₂ atmosphere and stirred at 100° C. overnight. The mixture was cooled down to rt, diluted with H₂O, extracted with EA (60 mL×2), washed with brine, dried over Na₂SO₄, filtered, and concentrated to dryness. The residue was purified by flash column chromatography to give the titled compound (65 mg, 28%). ¹H NMR (400 MHz, CDCl₃) δ 7.92 (d, J=8.4 Hz, 2H), 7.08 (d, J=8.4 Hz, 2H), 6.20-6.14 (m, 1H), 5.62-5.56 (m, 2H), 5.44-5.38 (m, 2H), 2,59 (s, 3H) ppm.

### Step C: 1-(4-(oxetan-3-ylmethyl)phenyl)ethan-1-one

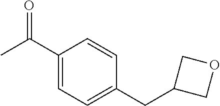

To a solution of 1-(4-(oxetan-3-ylidenemethyl)phenyl)ethan-1-one (60 mg, 0.32 mmol) in EA (5 mL) was added Pd/C (10 mg, 10%). The reaction was stirred at 25° C. under H₂ (balloon) overnight. The mixture was filtered through a pad of Celtic and the filtrate was concentrated to dryness to give the titled compound (55 mg, 91%). MS: M/e 191 (M+1)⁺.

### Step D: 1-(4-(oxetan-3-ylmethyl)phenyl)ethan-1-ol

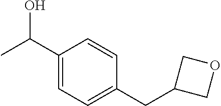

To a solution of 1-(4-(oxetan-3-ylmethyl)phenyl)ethan-1-one (55 mg, 0.289 mmol) in MeOH (3 mL) was added NaBH₄ (10 mg, 0.29 mmol) slowly at 0° C. The resulting mixture was stirred at room temperature for 1 hour. The reaction mixture was quenched with H₂O (10 mL) and extracted with EA (80 mL), washed with brine, dried over Na₂SO₄, filtered, and concentrated to dryness. The residue was purified by flash column chromatography to give the titled compound (40 mg, 71%). ¹H NMR (400 MHz, DMSO-dt) δ 7.23 (d, J=8.0 Hz, 2H), 7.10 (d, J=8.4 Hz, 2H), 5.06 (d, J=4.0 Hz, 1H), 4.70-4.56 (m, 3H), 4.32 (t, J=6.0 Hz, 2H), 3.28-3.16 (m, 1H), 2.91 (d, J=7.6 Hz, 2H), 1,28 (d, J=6.4 Hz, 3H) ppm.

### Step E: 2-(7-((2S,5R)-2,5-diethyl-4-(1-(4-(oxetan-3-ylmethyl)phenyl)ethyl)piperazin-1-yl)-4-methyl-5-oxo-4,5-dihydro-2H-pyrazolo[4,3-b]pyridin-2-yl)acetonitrile

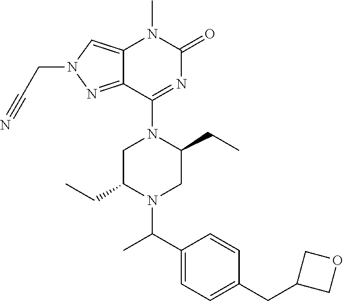 [SMILES: CC[C@H]1CN(C(C)c2ccc(CC3COC3)cc2)[C@H](CC)CN1c1nc(=O)n(C)c2cn(CC#N)nc12]

To a solution of 1-(4-(oxetan-3-ylmethyl)phenyl)ethan-1-ol (40 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (32 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-HPLC (Method A) to give the titled compound (3 mg, 6%). ¹H NMR (400 MHz, CD₃OD) δ 7.89 (s, 1H), 7.32-7.23 (m, 2H), 7.17-7.06 (m, 2H), 5.51 (s, 1H), 5.43 (s, 2H), 4.80-4.68 (m, 2H), 4.50-4.41 (m, 2H), 3.80-3.42 (m, 2H), 3.41 (s, 3H), 3.35-3.31 (m, 1H), 3.16-3.06 (m, 1H), 3.03-2.82 (m, 3H), 2.70-2.30 (m, 2H), 2.16-1.43 (m, 5H), 1.35-1,22 (m, 3H), 1.05-0.89 (m, 3H), 0.70-0.53 (m, 3H) ppm. MS: M/e 503 (M+1)⁺.

### Compound A184: 2-(7-((2S,5R)-4-(1-([1,2,4]triazolo[4,3-a]pyridin-7-yl)ethyl)-2,5-diethylpiperazin-1-yl)-4-methyl-5-oxo-4,5-dihydro-2H-pyrazolo[4,3-b]pyridin-2-yl)acetonitrile

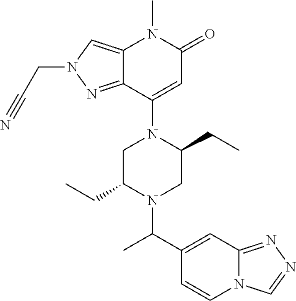 [SMILES: CC[C@H]1CN(C(C)c2ccn3cnnc3c2)[C@H](CC)CN1c1cc(=O)n(C)c2cn(CC#N)nc12]

### Step A: 1-([1,2,4]triazolo[4,3-a]pyridin-7-yl)ethan-1-one

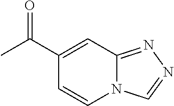

To a solution of 7-bromo-[1,2,4]triazolo[4,3-a]pyridine (2 g, 10 mmol) in toluene (20 mL) was added tributyl(1-ethoxyvinyl)stannane (4.74 g, 15 mmol) and Pd(PPh₂)₂Cl₂ (350 mg, 0.5 mmol). The reaction mixture was protected by N₂ atmosphere and stirred at 100° C. overnight. Then Pd(PPh₂)₂Cl₂ (350 mg, 0.5 mmol) and DMF (10 mL) were added. The reaction mixture was protected by N₂ atmosphere and stirred at 100° C. overnight. The mixture was cooled down to rt, diluted with H₂O, extracted with DCM (80 mL×3), washed with brine, dried over Na₂SO₄, filtered, and concentrated to dryness. The residue was dissolved in DCM (20 mL), 4 M HCl in dioxane (5 mL) was added and the reaction was stirred at 25° C. for 4 hours. The mixture was quenched with saturated NaHCO₃ solution, extracted with DCM (60 mL×3), washed with brine, dried over Na₂SO₄, filtered, and concentrated to dryness. The residue was purified by flash column chromatography to give the titled compound (320 mg, 20%). ¹H NMR (400 MHz, CDCl₃) δ 8.93 (s, 1H), 8.40 (s, 1H), 8.18 (d, J=7.6 Hz, 1H), 7.48 (d, J=7.6 Hz, 1H), 2.70 (s, 3H) ppm.

### Step B: 1-(11.2,4]triazolo[4,3-a]pyridin-7-yl)ethan-1-ol

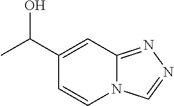

To a solution of 1-([1,2,4]triazolo[4,3-a]pyridin-7-yl)ethan-1-one (320 mg, 2 mmol) in MeOH/DCM (6 mL/1 mL) was added NaBH₄ (76 mg, 2 mmol) slowly at 0° C. The resulting mixture was stirred at room temperature for 1 hour. The reaction mixture was quenched with H₂O (30 mL) and extracted with DCM (80 mL). The water layer was collected, concentrated to dryness. The residue was purified by flash column chromatography to give the desired product (260 mg, 81%). ¹H NMR (400 MHz, DMSO-d₆) δ 9.19 (s, 1H), 8.50 (d, J=7.2 Hz, 1H), 7.60 (s, 1H), 6.97 (d, J=7.2 Hz, 1H), 5.49 (d, J=4.0 Hz, 1H), 4.81-4.70 (m, 1H), 1.36 (d, J=6.4 Hz, 3H) ppm.

### Step C: 2-(7-((2S,5R)-4-(1-([1,2,4]triazolo[4,3-a]pyridin-7-yl)ethyl)-2,5-diethylpiperazin-1-yl-4-methyl-5-oxo-4,5-dihydro-2H-pyrazolo[4,3-b]pyridin-2-yl)acetonitrile

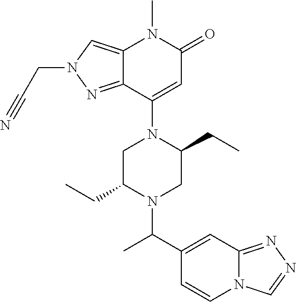 [SMILES: CC[C@H]1CN(C(C)c2ccn3cnnc3c2)[C@H](CC)CN1c1cc(=O)n(C)c2cn(CC#N)nc12]

To a solution of 1-([1,2,4]triazolo[4,3-a]pyridin-7-yl)ethan-1-ol (32 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness to give the titled compound (crude), which was further purified and separated into Compound A184a (9 mg) and Compound A184b (9 mg) by Prep-HPLC (Method A).

Compound A184a (the earlier peak): ¹H NMR (400 MHz, CD₃OD) δ 9.14 (s, 1H), 8.48 (d, J=6.8 Hz, 1H), 7.93 (s, 1H), 7.67 (s, 1H), 7.21 (d, J=7.2 Hz, 1H), 5.58 (s, 1H), 5.46 (s, 2H), 3.98-3.80 (m, 1H), 3.43 (s, 3H), 3.39-3.32 (m, 2H), 3.35-3.31 (m, 1H), 3.05-2.85 (m, 2H), 2,54-2,42 (m, 1H), 2.17-2.05 (m, 1H), 1.90-1.79 (m, 1H), 1.66-1.52 (m, 2H), 1.40 (d, J=6.8 Hz, 3H), 0.95 (t, J=7.6 Hz, 3H), 0.76 (t, J=7.6 Hz, 3H) ppm. MS: M/e 474 (M+1)⁺.

Compound A184b (the later peak): ¹H NMR (400 MHz, CD₃OD) 5 9.13 (s, 1H), 8.47 (d, J=6.8 Hz, 1H), 7.93 (s, 1H), 7.71 (s, 1H), 7.22 (d, J=7.2 Hz, 1H), 5.57 (s, 1H), 5.47 (s, 2H), 3.83-3.72 (m, 1H), 3.58-3.50 (m, 1H), 3.43 (s, 3H), 3.35-3.31 (m, 1H), 3.20-3.10 (m, 1H), 2.84-2.72 (m, 1H), 2,46-2.35 (m, 1H), 2.06-1.50 (m, 5H), 1.38 (d, 0.1=6.8 Hz, 3H), 1.03 (t, J=7.6 Hz, 3H), 0.68 (t, J=7.6 Hz, 3H) ppm. MS: M/e 474 (M+1)⁺.

### Compound A185: 2-(7-((2S,5R)₄-(1-(benzo[d][1,3]dioxol-5-yl)ethyl)-2,5-diethylpiperazin-1-yl)-4-methyl-5-oxo-4,5-dihydro-2H-pyrazolo[4,3-b]pyridin-2-yl)acetonitrile

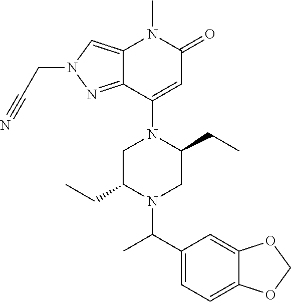 [SMILES: CC[C@H]1CN(C(C)c2ccc3c(c2)OCO3)[C@H](CC)CN1c1cc(=O)n(C)c2cn(CC#N)nc12]

### Step A: 1-(benzo[d][1.3]dioxol-5-yl)ethan-1-ol

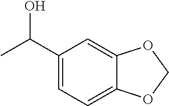

To a solution of 1-(benzo[d][1,3]dioxol-5-yl)ethan-1-one (60 mg, 0.36 mmol) in MeOH (3 mL) was added NaBH₄ (14 mg, 0.36 mmol) at room temperature and the resulting mixture was stirred at room temperature for 5 min. The reaction mixture was diluted with DCM and washed with water, dried over Na₂SO₄ and concentrated to give the titled compound (60 mg). MS: M/e 167 (M+1)⁺.

### Step B: 2-(74 (2S,5R)-4-(1-(benzo[d]1.3]dioxol-5-yl)ethyl)-2,5-diethylpiperazin-1-yl)-4-methyl-5-oxo-4,5-dihydro-2H-pyrazolo[4,3-b]pyridin-2-yl)acetonitrile

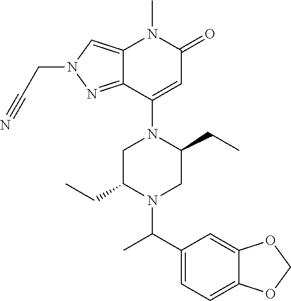 [SMILES: CC[C@H]1CN(C(C)c2ccc3c(c2)OCO3)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (2 mL) and was added 1-(benzo[d][1,3]dioxol-5-yl)ethan-1-ol (50 mg, 0.3 mmol), (cyanomethyl)trimethylphosphonium iodide (111 mg, 0.45 mmol) and DIPEA(197 mg, 1.5 mmol). The resulting mixture was stirred at 105° C. overnight. The reaction solvent was removed under reduce pressure. The resulting residue was purified by Prep-HPLC (Method A) to give the titled compound (1.5 mg)). ¹H NMR (400 MHz, CD₃OD) δ 7.92 (s, 1H), 6.91 (d, J=14.4 Hz, 1H), 6.85-6.71 (m, 2H), 5.92 (d, J=9.0 Hz, 2H), 5.55 (s, 1H), 5.47 (d, J=2,4 Hz, 2H), 4.69-4.10 (m, 2H), 3.67 (d, J=6.1 Hz, 1H), 3.57-3.46 (m, 1H), 3.43 (s, 3H), 3.18-2.38 (m, 3H), 2.20-1.46 (m, 4H), 1.30 (dd, J=13.0, 6.4 Hz, 3H), 0.98 (dt, J=20.4, 7.5 Hz, 3H), 0.69 (dt, J=20.4, 7.3 Hz, 3H) ppm. MS: M/e 477 (M+1)⁺.

### Compound A186: 2-(7-((2S,5R)-2,5-diethyl-4-(1-(phthalazin-6-yl)ethyl)piperazin-1-yl)-4-methyl-5-oxo-4,5-dihydro-2H-pyrazolo[4,3-b]pyridin-2-yl)acetonitrile

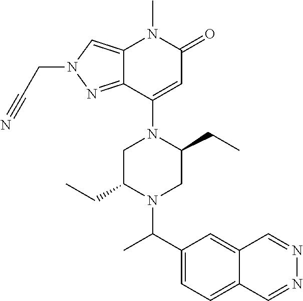 [SMILES: CC[C@H]1CN(C(C)c2ccc3cnncc3c2)[C@H](CC)CN1c1cc(=O)n(C)c2cn(CC#N)nc12]

### Step A: 1-(phthalazin-6-yl)ethan-1-one

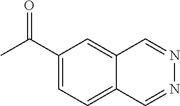

A mixture of 6-bromophthalazine (500 mg, 2,4 mmol), tributyl(1-ethoxyvinyl)stannane (957 mg, 2.6 mmol) and Pd(PPh₃)₂Cl₂ (170 mg, 0.24 mmol) in toluene (2 mL) was stirred at 100° C. under N₂ for 16 hours. The reaction mixture was concentrated and purified by flash column chromatography to give the titled compound (260 mg), The reaction mixture was diluted with ethyl acetate (5 mL) and HCl (2 mL, 3 M in ethyl acetate) in drops and the mixture was stirred at room temperature for 0.5 hour. The reaction was concentrated and adjusted to pH=8-9 with saturated NaHCO₃ solution, extracted with DCM (20 mL×2). The combined organic layers were washed with brine, dried over Na₂SO₄ and concentrated to give the titled compound (260 mg). MS: M/e 173 (M+1)⁺

### Step B: 1-(phthalazin-6-yl)ethan-1-ol

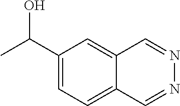

To a solution of 1-(phthalazin-6-yl)ethan-1-one (260 mg, 1.5 mmol) in MeOH (3 mL) was added NaBH₄ (34 mg, 0.9 mmol) at room temperature and the resulting mixture was stirred at room temperature for 5 min. The reaction mixture was diluted with DCM and washed with water, dried over Na₂SO₄ and purified by flash column chromatography to give the titled compound (45 mg, 25%). MS: M/e 175 (M+1)⁺.

### Step C: 2-(7-((2S,5R)-2,5-diethyl-4-(1-(phthalazin-6-yl)ethyl)piperazin-1-yl)-4-methyl-5-oxo-4,5-dihydro-2H-pyrazolo[4,3-b]pyridin-2-yl)acetonitrile

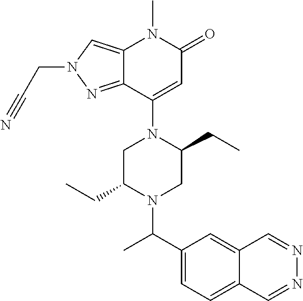 [SMILES: CC[C@H]1CN(C(C)c2ccc3cnncc3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (25 mg, 0.076 mmol) in CH₃CN (2 mL) and was added 1-(phthalazin-6-yl)ethan-1-ol (25 mg, 0.14 mmol), (cyanomethyl)trimethylphosphonium iodide (55 mg, 0.22 mmol) and DIPEA(98 mg, 1.5 mmol).The resulting mixture was stirred at 105° C. overnight. The reaction solvent was removed under reduce pressure. The resulting residue was purified by flash column chromatography to give the titled compound(crude), which was further purified to give the titled Compound A186a (0.3 mg) and Compound A186b (0.3 mg) by Prep-HPLC (Method A).

Compound A186a (the earlier peak): ¹H NMR (400 MHz, CD₃OD) δ 9.60 (d, J=9.0 Hz, 2H), 8.26-8.11 (m, 3H), 7.92 (s, 1H), 5.57 (s, 1H), 5.45 (s, 2H), 4.60 (s, 2H), 4.12 (d, J=6.4 Hz, 1H), 3.43 (s, 3H), 3.34 (d, J=17.4 Hz, 1H), 3.20-2.91 (m, 2H), 2.37 (s, 1H), 2.23-1.57 (m, 4H), 1.45 (d, J=6.4 Hz, 3H), 0.97 (t, J=7.4 Hz, 3H), 0.68 (t, J=7.3 Hz, 3H) ppm. MS: M/e 485 (M+1)⁺.

Compound A186b (the later peak): ¹H NMR (400 MHz, CD₃OD) 9.58 (s, 2H), 8.32-8.11 (m, 3H), 7.93 (s, 1H), 5.56 (s, 1H), 5.46 (s, 2H), 4.59 (s, 2H), 3.96 (d, J=6.3 Hz, 1H), 3.57 (d, J=14.0 Hz, 1H), 3.43 (s, 3H), 3.28-2.70 (m, 2H), 2.29 (d, J=12.1 Hz, 1H), 1.77 (t, J=66.9 Hz, 4H), 1.43 (d, J=6.6 Hz, 3H), 1.06 (t, J=7.3 Hz, 3H), 0.55 (t, .=7.4 Hz, 3H) ppm. MS: M/e 485 (M+1)⁺.

### Compound A187:-2-(7-((2S,5R)-4-(1-(6-aminopyridin-3-yl)ethyl)-2,5-diethylpiperazin-1-yl)-4-methyl-5-oxo-4,5-dihydro-2H-pyrazolo[4,3-b]pyridin-2-yl)acetonitrile

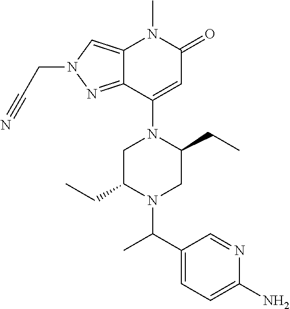 [SMILES: CC[C@H]1CN(C(C)c2ccc(N)nc2)[C@H](CC)CN1c1cc(=O)n(C)c2cn(CC#N)nc12]

### Step A: benzyl (5-(1-hydroxyethyl)pyridin-2-yl)carbamate

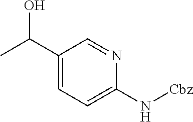 [SMILES: CC(O)c1ccc(NC(=O)OCc2ccccc2)nc1]

To a solution of benzyl (5-acetylpyridine-2-yl)carbamate (300 mg, 1.11 mmol) in MeOH (15 mL) was added NaBH₄ (42 mg, 1.11 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (270 mg, 89%). MS: M/e 273 (M+1)⁺.

### Step B: benzyl (5414 (2R,5S)₄-(2-(cyanomethyl)-4-methyl-5-oxo-4,5-dihydro-2H-pyrazolo[4,3-b]pyridin-7-yl)-2,5-diethylpiperazin-1-yl)ethyl)pyridin-2-yl)carbamate

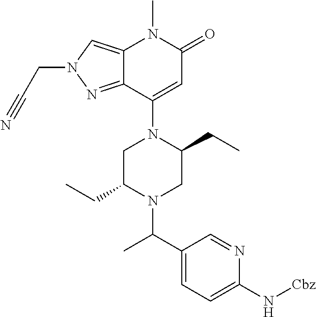 [SMILES: CC[C@H]1CN(C(C)c2ccc(NC(=O)OCc3ccccc3)nc2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (200 mg, 0.61 mmol) in CHCN (5 mL) and was added benzyl (5-(1-hydroxyethyl)pyridin-2-yl)carbamate (166 mg, 0.61 mmol), (cyanomethyl)trimethylphosphonium iodide (443 mg, 1.83 mmol) and DIPEA (393 mg, 3.05 mmol). The resulting mixture was stirred at 105° C. overnight. The reaction solvent was removed under reduce pressure to afford crude product. The resulting residue was purified to give the titled compound (120 mg, 34%). MS: M/e 583 (M+1)⁺.

### Step C: 2-(7-((2S,5R)₄-(1-(6-aminopyridin-3-yl)ethyl)-2,5-diethylpiperazin-1-yl)-4-methyl-5-oxo-4,5-dihydro-2H-pyrazolo[4,3-b]pyridin-2-yl)acetonitrile

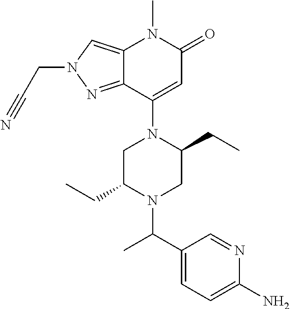 [SMILES: CC[C@H]1CN(C(C)c2ccc(N)nc2)[C@H](CC)CN1c1cc(=O)n(C)c2cn(CC#N)nc12]

To a solution of benzyl (5-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)pyridin-2-yl)carbamate (120 mg, 0.21 mmol) in THF (5 mL) was added Pd/C (30 mg, 10%). The reaction mixture was stirred under H₂ atmosphere (1 atm) at room for 3 hours. The reaction mixture was filtered and concentrated to dryness to give the titled compound(crude), which was further purified and separated into Compound A187a (0.7 mg) and Compound A187b (0.9 mg) by Prep-HPLC (Method A).

Compound A187a (the earlier peak): ¹H NMR (400 MHz, CD₃OD) δ 7.92 (s, 1H), 7.80 (s, 1H), 7.63 (d, J=8.6 Hz, 1H), 6.70 (d, J=8.7 Hz, 1H), 5.55 (s, 1H), 5.47 (s, 2H), 4.60 (s, 3H), 3.69 (d, J=7.0 Hz, 1H), 3.43 (s, 3H), 2.97 (d, J=10.5 Hz, 1H), 2.87 (d, J=8.3 Hz, 1H), 2,48 (s, 1H), 2.16-2.04 (m, 1H), 1.80 (d, J=7.1 Hz, 1H), 1.51 (d, .=8.0 Hz, 2H), 1.35 (d, J=6.5 Hz, 3H), 0.94 (t, J=7.3 Hz, 3H), 0.76 (t, J=7.3 Hz, 3H). MS: M/e 449 (M+1)⁺.

Compound A187b (the later peak): ¹H NMR (400 MHz, CD₃OD) δ 7.93 (s, 1H), 7.82 (s, 1H), 7.67 (d, J=8.8 Hz, 1H), 6.69 (d, J=8.6 Hz, 1H), 5.55 (s, 1H), 5.47 (s, 2H), 4.60 (s, 3H), 3.54 (d, J=6.3 Hz, 1H), 3.47 (d, J=12,4 Hz, 1H), 3.43 (s, 3H), 3.11 (d, J=10.1 Hz, 1H), 2.65 (d, J=8.5 Hz, 1H), 2,44 (d, J=12.3 Hz, 1H), 1.95-1.83 (m, 1H), 1.70-1.60 (m, 2H), 1.31 (d, J=6.6 Hz, 3H), 1.01 (t, J=7.3 Hz, 3H), 0.68 (t, J=7.4 Hz, 3H). MS: M/e 449 (M+1)⁺.

### Compound A188: 2-(7-((2S,5R)-2,5-diethyl-4-(1-(1-methylisoquinolin-6-yl)ethyl)piperazin-1-yl)-4-methyl-5-oxo-4,5-dihydro-2H-pyrazolo[4,3-b]pyridin-2-yl)acetonitrile

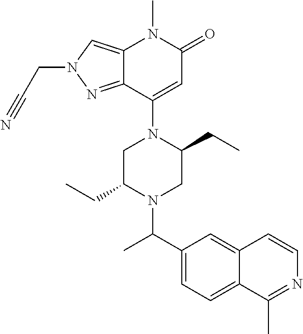 [SMILES: CC[C@H]1CN(C(C)c2ccc3c(C)nccc3c2)[C@H](CC)CN1c1cc(=O)n(C)c2cn(CC#N)nc12]

### Step A: 6-bromo-1-methylisoquinoline

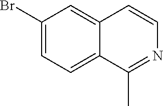

A mixture of 6-bromo-1-chloroisoquinoline (500 mg, 2.07 mmol) in THF (10 mL) at 0° C. was added Ferric acetylacetonate (73 mg, 0.207 mol) and methyl magnesium bromide (0.76 mL, 3M, 2.28 mol). The reaction mixture was stirred at room temperature for 10 mins. The reaction was quenched by adding aqueous NH₄Cl. The resulting mixture was extracted with ethyl acetate. Then the resulting organic layer was concentrated to dryness. The resulting residue was purified by flash column chromatography to give the titled compound (270 mg, 58%). MS: M/e 222 (M+1)⁺.

### Step B: 1-(1-methylisoquinolin-6-yl)ethan-1-one

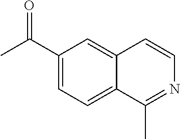

A mixture of 6-bromo-1-methylisoquinoline (270 mg, 1,2 mmol), tributyl(1-ethoxyvinyl)stannane (884 mg, 2,4 mmol) and Pd(PPh₃)₂Cl₂ (86 mg, 0.12 mmol) in toluene (2 mL) was stirred at 100° C. under N₂ for 16 hours. The reaction mixture was concentrated and purified by flash column chromatography to give the titled compound (250 mg), The reaction mixture was diluted with ethyl acetate (5 mL) and HCl (2 mL, 3 M in ethyl acetate) in drops and the mixture was stirred at room temperature for 0.5 hour. The reaction was concentrated and adjusted to pH=8-9 with saturated NaHCO₃ solution, extracted with DCM (20 mL×2). The combined organic layers were washed with brine, dried over Na₂SO₄ and concentrated to give the titled compound (200 mg). MS: M/e 186 (M+1)⁺

### Step C: 1-(1-methylisoquinolin-6-yl)ethan-1-ol

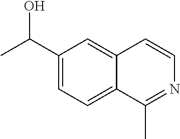

To a solution of 1-(1-methylisoquinolin-6-yl)ethan-1-one (200 mg, 1.08 mmol) in MeOH (3 mL) was added NaBH₄ (41 mg, 1.08 mmol) at room temperature and the resulting mixture was stirred at room temperature for 5 min. The reaction mixture was diluted with DCM and washed with water, dried over Na₂SO₄ and concentrated to give the titled compound (150 mg). MS: M/e 188 (M+1)⁺.

### Step D: 2-(7-((2S,5R)-2,5-diethyl-4-(1-(1-methylisoquinolin-6-yl)ethyl)piperazin-1-yl)-4-methyl-5-oxo-4,5-dihydro-2H-pyrazolo[4,3-b]pyridin-2-yl)acetonitrile

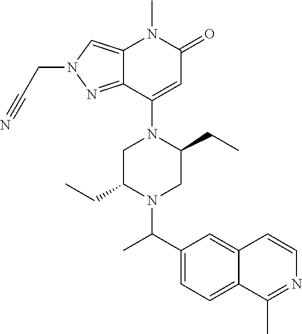 [SMILES: CC[C@H]1CN(C(C)c2ccc3c(C)nccc3c2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (2 mL) and was added 1-(1-methylisoquinolin-6-yl)ethan-1-ol (43 mg, 0.23 mmol), (cyanomethyl)trimethylphosphonium iodide (111 mg, 0.45 mmol) and DIPEA(197 mg, 1.5 mmol).The resulting mixture was stirred at 105° C. overnight. The reaction solvent was removed under reduce pressure. The resulting residue was purified by flash column chromatography to give the titled compound(crude), which was further purified and separated into Compound A188a (4 mg) and Compound A188b (4 mg) by Prep-HPLC (Method A).

Compound A188a (the earlier peak): ¹H NMR (400 MHz, CD₃OD) δ 8.26 (d, J=6.2 Hz, 2H), 7.92 (s, 1H), 7.86-7.81 (m, 2H), 7.67 (d, J=5.9 Hz, 1H), 5.56 (s, 1H), 5.45 (s, 2H), 4.75-4.08 (m, 2H), 4.00 (q, J=6.4 Hz, 1H), 3.43 (s, 3H), 3.33 (s, 1H), 3.08 (d, J=12.0 Hz, 1H), 2.95 (s, 3H), 2.91 (d, J=3.8 Hz, 1H), 2,41 (d, J=8.0 Hz, 1H), 2.17 (dt, J=15.1, 7.7 Hz, 1H), 1.92-1.80 (m, 1H), 1.68-1.51 (m, 2H), 1.44 (d, J=6.4 Hz, 3H), 0.98 (t, J=7.4 Hz, 3H), 0.65 (t, J=7.3 Hz, 3H) ppm. MS: M/e 498 (M+1)⁺.

Compound A188b (the later peak): ¹H NMR (400 MHz, CD₃OD) δ 8.24 (t, J=7.4 Hz, 2H), 7.91 (d, J=9.7 Hz, 2H), 7.84 (d, J=8.5 Hz, 1H), 7.64 (d, J=5.9 Hz, 1H), 5.55 (s, 1H), 5.46 (s, 2H), 4.76-3.99 (m, 2H), 3.90-3.78 (m, 1H), 3.56 (d, J=12.7 Hz, 1H), 3.43 (s, 3H), 3.22 (d, J=10.2 Hz, 1H), 2.94 (s, 3H), 2.72 (d, J=9.0 Hz, 1H), 2.35 (d, J=12.3 Hz, 1H), 1.99 (d, J=6.5 Hz, 1H), 1.79-1.56 (m, 3H), 1.41 (d, J=6.5 Hz, 3H), 1.05 (t, J=7.3 Hz, 3H), 0.57 (t, J=7.4 Hz, 3H) ppm. MS: M/e 498 (M+1)⁺.

### Compound A189: 2-(7-((2S,5R)-2,5-diethyl-4-(1-(4-((4-methoxypiperidin-1-yl)methyl)phenyl)ethyl)piperazin-1-yl)-4-methyl-5-oxo-4,5-dihydro-2H-pyrazolo[4,3-b]pyridin-2-yl)acetonitrile

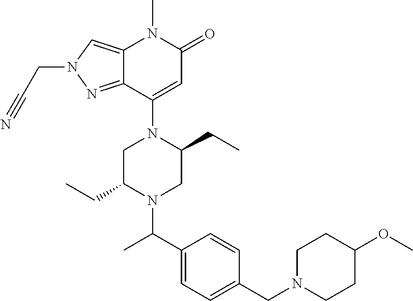 [SMILES: CC[C@H]1CN(C(C)c2ccc(CN3CCC(OC)CC3)cc2)[C@H](CC)CN1c1cc(=O)n(C)c2cn(CC#N)nc12]

### Step A: 1-4-bromobenzyl)-4-methoxypiperidine

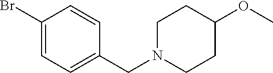 [SMILES: COC1CCN(Cc2ccc(Br)cc2)CC1]

A solution of 4-bromobenzaldehyde (500 mg, 2.70 mmol), 4-methoxypiperidine (373 mg, 3.24 mmol) and STAB (1.15 g, 5.42 mmol) in DCM (10 ml) was stirred at rt overnight. The solution was washed with aq. NaHCO₃ (10 ml×2), dried over Na₂SO₄ and concentrated to dryness. The resulting residue was purified by flash column chromatography with 0-10/0 MeOH in DCM to give the titled compound (767 mg, 100%). MS: M/e 284,286 (M+1)⁺.

### Step B: 1-(4-((4-methoxypiperidine-1-yl)methyl)phenyl)ethan-1-one

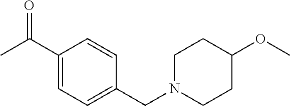 [SMILES: COC1CCN(Cc2ccc(C(C)=O)cc2)CC1]

A solution of 1-(4-bromobenzyl)-4-methoxypiperidine (350 mg, 1,23 mmol), ethyl tributylstannanecarboxylate (667 mg, 1.85 mmol) and Pd(PPh₃)₂Cl₂ (43 mg, 0.061 mmol) in toluene(6 ml) was stirred at 100° C. overnight. The solution was cooled to RT. HCl/1,4-dioxane (4M, 2 ml) was added and stirred at RT for 10 min. The solution was diluted with EtOAc (15 ml), washed with aq. Na₂CO₃ (10 ml×2), dried over Na₂SO₄ and concentrated to dryness. The resulting residue was purified by flash column chromatography with 0-10% MeOH in DCM to give the titled compound (170 mg, 56%). MS: We 248 (M+1)⁺.

### Step C: 1-(4-((4-methoxypiperidine-1-yl)methyl)phenyl)ethan-1-ol

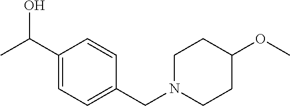 [SMILES: COC1CCN(Cc2ccc(C(C)O)cc2)CC1]

A solution of 1-(4-((4-methoxypiperidin-1-yl)methyl)phenyl)ethan-1-one (170 mg, 0.69 mmol) and NaBH₄ (26 mg, 0.68 mmol) in MeOH (2 ml) was stirred at RT for 15 min. The solution was diluted with EtOAc(10 ml), washed with brine (10 ml×2), dried over Na₂SO₄ and concentrated to dryness to give the titled compound (170 mg, 99%). MS: We 250 (M+1)⁺.

### Step D: 2-(7-((2S,5R)-2,5-diethyl-4-(1-(4-((4-methoxypiperidin-1-yl)methyl)phenyl)ethyl)piperazin-1-yl)-4-methyl-5-oxo-4,5-dihydro-2H-pyrazolo[4,3-b]pyridin-2-yl)acetonitrile

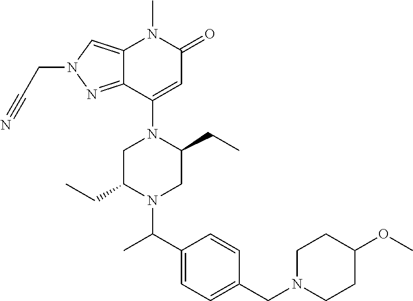 [SMILES: CC[C@H]1CN(C(C)c2ccc(CN3CCC(OC)CC3)cc2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (75 mg, 0.23 mmol), 1-(4-((4-methoxypiperidin-1-yl)methyl)phenyl)ethan-1-ol (170 mg, 0.68 mmol), (cyanomethyl)trimethylphosphonium iodide (167 mg, 0.69 mmol) and DIPEA (295 mg, 2.29 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled Compound A189 (90 mg), which was further separated into Compound A189a (3.5 mg) and Compound A189b (4.9 mg) by Prep-HPLC (Method A).

Compound A189a (the earlier peak): ¹H NMR (400 MHz, DMSO-d6): δ 8.16 (s, 1.8H), 7.97 (s, 1H), 7.27 (q, J=7.8 Hz, 4H), 5.61 (s, 2H), 5.39 (s, 1H), 3.67 (q, J=6.3 Hz, 2H), 3.46 (s, 3H), 3.27 (s, 3H), 3.21 (s, 3H), 3.17 (s, 1H), 3.10 (d, J=12.2 Hz, 1 H), 2.90 (d, J=12.1 Hz, 1H), 2.74 (d, J=10.5 Hz, 1H), 2.65 (d, J=10.8 Hz, 2H), 2.31 (s, 1H), 2.11 (t, J=9.9 Hz, 2H), 2.06-1.95 (m, 1H), 1.81 (d, J=11.2 Hz, 2H), 1.67-1.56 (m, 1H), 1.48-1.37 (m, 4H), 1,27 (d, J=6.2 Hz, 3H), 0.86 (t, J=7.4 Hz, 3H), 0.58 (t, J=7.2 Hz, 3H) ppm. MS: M/e 560 (M+1)⁺.

Compound A189b (the later peak): ¹H NMR (400 MHz, DMSO-d6): δ 8.25 (s, 1.65H), 7.98 (s, 1H), 7.30 (d, J=7.8 Hz, 2H), 7.22 (d, J=7.8 Hz, 2H), 5.61 (s, 2H), 5.37 (s, 1H), 3.53 (q, J=6.3 Hz, 1H), 3.42 (s, 2H), 3.33 (d, J=12.0 Hz, 1H), 3.27 (s, 3H), 3.20 (s, 3H), 3.17-3.11 (m, 1H), 3.04 (d, J=10.1 Hz, 1H), 2.60 (s, 2H), 2,50-2,45 (m, 3H), 2.22 (d, J=12.2 Hz, 1H), 2.05 (t, J=9.6 Hz, 2H), 1.92-1.75 (m, 3H), 1.63-1.50 (m, 1H), 1.49-1.33 (m, 4H), 1,24 (d, J=6.2 Hz, 3H), 0.94 (t, J=7.1 Hz, 3H), 0.48 (t, J=7.0 Hz, 3H) ppm. MS: M/e 560 (M+1)⁺.

### Compound A190: 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)phenyl)acetonitrile

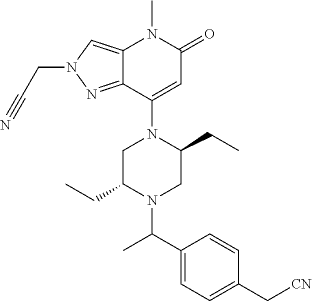 [SMILES: CC[C@H]1CN(C(C)c2ccc(CC#N)cc2)[C@H](CC)CN1c1cc(=O)n(C)c2cn(CC#N)nc12]

### Step A: 2-(4-acetylphenyl acetonitrile

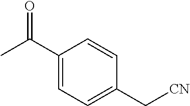

A mixture of 2-(4-bromophenyl)acetonitrile (392 mg, 2 mmol), tributyl(1-ethoxyvinyl)stannane (1.08 g, 3 mmol) and Pd(PPh₃)₂Cl₂ (140 mg, 0.2 mmol) in toluene (10 mL) was stirred at 100° C. under N₂ overnight. To the resulting solution was added HCl (0.5 mL, con.) in drops and the mixture was stirred at room temperature for 1 hour. The mixture was diluted with EtOAc (20 mL), treated with saturated NaHCO₃ aq. to pH ˜8, washed with brine (20 mL×3), dried and concentrated to dryness. The resulting residue was purified by flash column chromatography(EtOAc:PE=0-50% in 30 minutes) to give the titled compound (170 mg, 53%). MS: M/e 160 (M+1)⁺.

### Step B: 2-(4-(1-hydroxyethyl)phenyl)acetonitrile

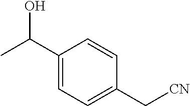

To a solution of 2-(4-acetylphenyl)acetonitrile (160 mg, 1 mmol) in MeOH (4 mL) was added NaBH₄ (30 mg, 0.8 mmol) at room temperature and the mixture was stirred at room temperature for 1 hour. The resulting residue was treated with water (20 mL), extracted with DCM (10 mL×3). The combined organic layers were washed with brine (20 mL), dried over Na₂SO₄ and concentrated to dryness. The resulting residue (140 mg, crude) was used in the next step. MS: M/e 162 (M+1)⁺.

### Step C: 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)phenyl))acetonitrile

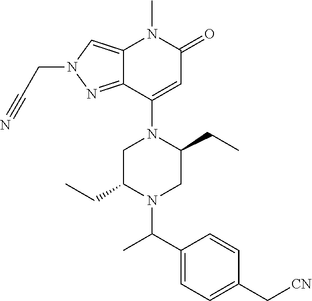 [SMILES: CC[C@H]1CN(C(C)c2ccc(CC#N)cc2)[C@H](CC)CN1c1cc(=O)n(C)c2cn(CC#N)nc12]

A mixture of 2-(4-(1-hydroxyethyl)phenyl)acetonitrile (24 mg, 0.15 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (48 mg, 0.2 mmol) and DIPEA (51 mg, 0.4 mmol) in MeCN (2 mL) was stirred at 100° C. overnight. The reaction mixture was diluted with EA (20 mL), washed with brine (10 mL×3), dried over Na₂SO₄ and concentrated to dryness. The resulting residue was purified by Prep-TLC (DCM:MeOH=13:1) to give the titled compound (30 mg, 63%). ¹H NMR (400 MHz, CD₃OD) δ 7.92 (d, J=2,5 Hz, 1H), 7.49-7.38 (m, 2H), 7.33 (t, J=8.7 Hz, 2H), 5.55 (s, 1H), 5.46 (d, J=3.7 Hz, 2H), 3.89 (d, J=5.2 Hz, 2H), 3.80-3.57 (m, 1H), 3.54-3.37 (m, 4H), 3.25 (s, 2H), 3.17-3.00 (m, 1H), 2.90-2,59 (m, 1H), 2,47-2.31 (m, 1H), 2.17-1.90 (m, 1H), 1.88-1.48 (m, 3H), 1.32 (dd, J=13.4, 6.6 Hz, 3H), 0.99 (dt, J=24.7, 7.5 Hz, 3H), 0.66 (dt, J=25.6, 7.3 Hz, 3H) ppm. MS: M/e 472 (M+1)⁺.

### Compound A191: 4-(1-((2R,5S)-4-(2-(cyanomethyl)-4-methyl-5-oxo-4,5-dihydro-2H-pyrazolo[4,3-b]pyridin-7-y)-2,5-diethylpiperazin-1-yl)ethyl)benzonitrile

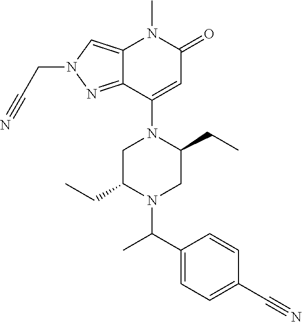 [SMILES: CC[C@H]1CN(C(C)c2ccc(C#N)cc2)[C@H](CC)CN1c1cc(=O)n(C)c2cn(CC#N)nc12]

### Step A: 4-(1-hydroxyethyl)-4benzonitrile

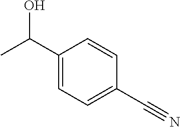

To a solution of 4-acetylbenzonitrile (145 mg, 1 mmol) in MeOH (3 mL) was added NaBH₄ (30 mg, 0.8 mmol) at room temperature and the mixture was stirred at room temperature for 1 hour. The resulting residue was treated with water (20 mL), extracted with DCM (10 mL×3). The combined organic layers were washed with brine (10 mL), dried over Na₂SO₄ and concentrated to dryness. The resulting residue (120 mg, crude) was used in the next step. MS: M/e 148 (M+1)”.

### Step B: 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)benzonitrile

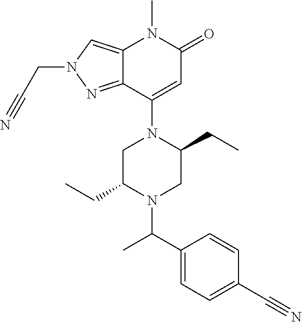 [SMILES: CC[C@H]1CN(C(C)c2ccc(C#N)cc2)[C@H](CC)CN1c1cc(=O)n(C)c2cn(CC#N)nc12]

A mixture of 4-(1-hydroxyethyl)benzonitrile (22 mg, 0.15 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol), (cyanomethyl)trimethylphosphonium iodide (48 mg, 0.2 mmol) and DIPEA (51 mg, 0.4 mmol) in MeCN (2 mL) was stirred at 100° C. overnight. The reaction mixture was diluted with EA (20 mL), washed with brine (10 mL×3), dried over Na₂SO₄ and concentrated to dryness. The resulting residue was purified by Prep-TLC (MeOH:DCM=0-10% in 30 minutes) to give the titled compound (19 mg, 41%). ¹H NMR (400 MHz, CD₃OD) δ 7.92 (d, J=2.2 Hz, 1H), 7.70 (t, J=8.2 Hz, 2H), 7.60 (dd, J=10.9, 8.4 Hz, 2H), 5.56 (s, 1H), 5.46 (d, J=3.3 Hz, 2H), 3.91-3.66 (m, 1H), 3.57-3.35 (m, 4H), 3.15 (d, J=12.1 Hz, 2H), 3.05-2.83 (m, 1H), 2.68 (d, J=9.1 Hz, 0.5H), 2.39-2.24 (m, 1H), 2.20-2.04 (m, 0.5H), 2.01-1.77 (m, 1H), 1.74-1.47 (m, 3H), 1.32 (dt, J=15.6, 7.7 Hz, 3H), 0.99 (dt, J=26.5, 7.3 Hz, 3H), 0.67 (dt, J=30.5, 7.4 Hz, 3H) ppm. MS: M/e 458 (M+1)⁺.

### Compound A192: 2-(7-((2S,5R)-4-(1-(4-((1,1-dioxidothiomorpholino)methyl)phenyl)ethyl)-2,5-diethylpiperazin-1-yl)-4-methyl-5-oxo-4,5-dihydro-2H-pyrazolo[4,3-b]pyridin-2-yl)acetonitrile

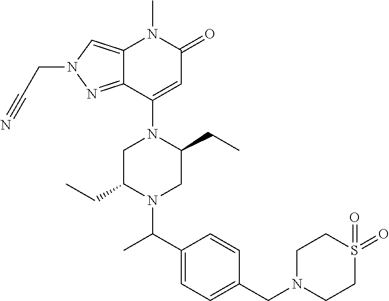 [SMILES: CC[C@H]1CN(C(C)c2ccc(CN3CCS(=O)(=O)CC3)cc2)[C@H](CC)CN1c1cc(=O)n(C)c2cn(CC#N)nc12]

### Step A: 1-(4-((1,1-dioxidothiomorpholino)methyl)phenyl)ethan-1-one

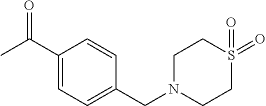 [SMILES: CC(=O)c1ccc(CN2CCS(=O)(=O)CC2)cc1]

To a solution of 1-(4-(bromomethyl)phenyl)ethan-1-one (212 mg, 1 mmol) and thiomorpholine 1,1-dioxide (135 mg, 1 mmol) in CH₃CN (4 mL) was added K₂CO₃ (276 mg, 2 mmol). The reaction was stirred at 25° C. overnight. The mixture was diluted with H₂O, extracted with EA (80 mL), washed with brine, dried over Na₂SO₄, filtered, and concentrated to dryness. The residue was purified by flash column chromatography to give the titled compound (230 mg, 85%). MS: M/e 268 (M+1)⁺.

### Step B: 4-(4-(1-hydroxyethyl)benzyl)thiomorpholine 1.1-dioxide

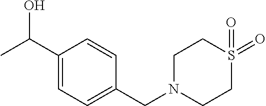 [SMILES: CC(O)c1ccc(CN2CCS(=O)(=O)CC2)cc1]

To a solution of 1-(4-((1,1-dioxidothiomorpholino)methyl)phenyl)ethan-1-one (230 mg, 0.85 mmol) in MeOH/DCM (5 mL/3 mL) was added NaBH₄ (32 mg, 0.85 mmol) slowly at 0° C. The resulting mixture was stirred at room temperature for 1 hour. The reaction mixture was quenched with H₂O (10 mL) and extracted with DCM (60 mL), washed with brine, dried over Na₂SO₄, filtered, and concentrated to dryness. The residue was purified by flash column chromatography to give the desired product (135 mg, 58%). ¹H NMR (400 MHz, DMSO-d₆) δ 7.34-7.22 (m, 4H), 5.11 (d, J=4.0 Hz, 1H), 4.74-4.64 (m, 1H), 3.63 (s, 2H), 3.14-3.06 (m, 4H), 2.92-2.78 (m, 4H), 1.31 (d, J=6.4 Hz, 3H) ppm.

### Step C: 2-(7-((2S,5R)-4-(1-(4-((1.1-dioxidothiomorpholino)methyl)phenyl)ethyl)-2,5-diethylpiperazin-1-yl)-4-methyl-5-oxo-4,5-dihydro-2H-pyrazolo[4,3-b]pyridin-2-yl)acetonitrile

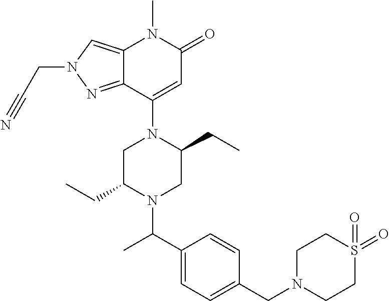 [SMILES: CC[C@H]1CN(C(C)c2ccc(CN3CCS(=O)(=O)CC3)cc2)[C@H](CC)CN1c1cc(=O)n(C)c2cn(CC#N)nc12]

To a solution of 4-(4-(1-hydroxyethyl)benzyl)thiomorpholine 1,1-dioxide (54 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.6 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-HPLC (Method A) to give the titled compound (10 mg, 17%). ¹H NMR (400 MHz, CD₃OD) δ 7.92 (s, 1H), 7.42-7.28 (m, 4H), 5.54 (s, 1H), 5.46 (s, 2H), 3.85-3.60 (m, 3H), 3.53-3.45 (m, 1H), 3.43 (s, 3H), 3.35-3.31 (m, 1H), 3.21-2.85 (m, 10.5H), 2.68-2,58 (m, 0.5H), 2,51-2.35 (m, 1H), 2.17-1.45 (m, 4H), 1.40-1,28 (m, 3H), 1.06-0.90 (m, 3H), 0.73-0.55 (m, 3H) ppm. MS: M/e 580 (M+1)⁺.

### Compound A193:4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-N-methylbenzamide

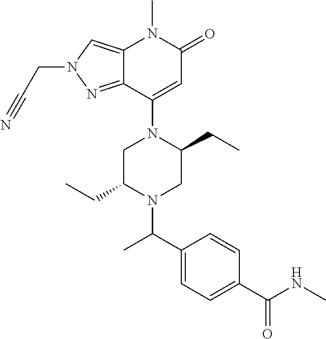 [SMILES: CC[C@H]1CN(C(C)c2ccc(C(=O)NC)cc2)[C@H](CC)CN1c1cc(=O)n(C)c2cn(CC#N)nc12]

### Step A: 4-acetyl-N-methylbenzamide

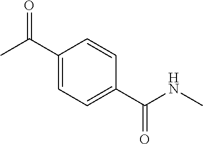

To a solution of 4-acetylbenzoic acid (164 mg, 1 mmol), methanamine hydrochloride (100 mg, 1.5 mmol) and HATU (570 mg, 1.5 mmol) in DCM (10 mL) was added DIEA (387 mg, 3 mmol). The reaction mixture was stirred at 25° C. overnight. The mixture was diluted with water, extracted with DCM (40 mL×2), washed with brine, dried over Na₂SO₄, filtered, and concentrated to dryness (200 mg, crude). MS: We 178 (M+1)⁺.

### Step B: 4-(1-hydroxyethyl)-N-methylbenzamide

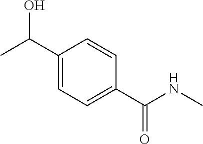

To a solution of 4-acetyl-N-methylbenzamide (200 mg, crude) in MeOH (5 mL) was added NaBH₄ (38 mg, 1 mmol) slowly at 0° C. The resulting mixture was stirred at room temperature for 1 hour. The reaction mixture was quenched with H₂O (0.5 mL) and concentrated to dryness. The residue was purified by flash column chromatography to give the desired product (115 mg, 64% for two steps). ¹H NMR (400 MHz, DMSO-d₄) δ 8.35 (d, J=4.0 Hz, 1H), 7.77 (d, J=8.4 Hz, 2H), 7.40 (d, J=8.0 Hz, 2H), 5.24 (d, J=4.4 Hz, 1H), 4.80-4.70 (m, 1H), 2.77 (d, J=3.6 Hz, 3H), 1.31 (d, J=6.0 Hz, 3H) ppm.

### Step C: 4-(1-((2R,5S)-4-(2-(cyanomethyl-4-methyl-5-oxo-4,5-dihydro-2H-pyrazolo[4,3-b]pyridin-7-yl)-2,5-diethylpiperazin-1-yl)ethyl-N-methylbenzamide

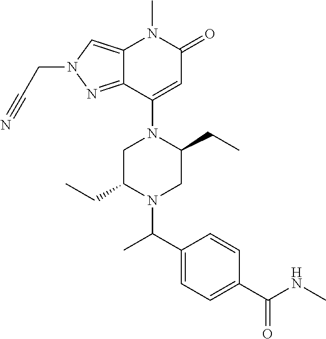 [SMILES: CC[C@H]1CN(C(C)c2ccc(C(=O)NC)cc2)[C@H](CC)CN1c1cc(=O)n(C)c2cn(CC#N)nc12]

To a solution of 4-(1-hydroxyethyl)-N-methylbenzamide (36 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄ and filtered. The resulting filtrate was concentrated to dryness to give the titled compound (crude), which was further purified and separated into Compound A193a (11 mg) and Compound A193b (13 mg) by Prep-HPLC (Method A).

Compound A193a (the earlier isomer): ¹H NMR (400 MHz, CD₃OD) δ 7.92 (s, 1H), 7.79 (d, J=8.4 Hz, 2H), 7.48 (d, J=8.4 Hz, 2H), 5.55 (s, 1H), 5.47 (s, 2H), 3.88-3.76 (m, 1H), 3.43 (s, 3H), 3.35-3.31 (m, 2H), 3.28-3.24 (m, 1H), 3.06-2.98 (m, 1H), 2.92 (s, 3H), 2.91-2.84 (m, 1H), 2,45-2.35 (m, 1H), 2.17-2.05 (m, 1H), 1.89-1.75 (m, 1H), 1.59-1.46 (m, 2H), 1.35 (d, J=6.8 Hz, 3H), 0.96 (t, J=7.2 Hz, 3H), 0.69 (t, J=7.2 Hz, 3H) ppm. MS: M/e 490 (M+1)⁺.

Compound A193b (the later isomer): ¹H NMR (400 MHz, CD₃OD) δ 7.92 (s, 1H), 7.78 (d, J=8.4 Hz, 2H), 7.50 (d, J=8.4 Hz, 2H), 5.55 (s, 1H), 5.47 (s, 2H), 3.72-3.62 (m, 1H), 3.55-3.46 (m, 1H), 3.43 (s, 3H), 3.35-3.31 (m, 2H), 3.20-3.08 (m, 1H), 2.92 (s, 3H), 2.71-2.60 (m, 1H), 2.38-2.28 (m, 1H), 2.00-1.85 (m, 1H), 1.75-1.50 (m, 3H), 1.31 (d, J=6.0 Hz, 3H), 1.02 (t, J=7.2 Hz, 3H), 0.62 (t, 0.1=7.6 Hz, 3H) ppm. MS: M/e 490 (M+1)⁺.

### Compound A194: 2-(7-((2S,5R)-2,5-diethyl-4-(1-(4-(4-methoxypiperidine-1-carbonyl)phenyl)ethyl)piperazin-1-yl-4-methyl-5-oxo-4,5-dihydro-2H-pyrazolo[4,3-b]pyridin-2-yl)acetonitrile

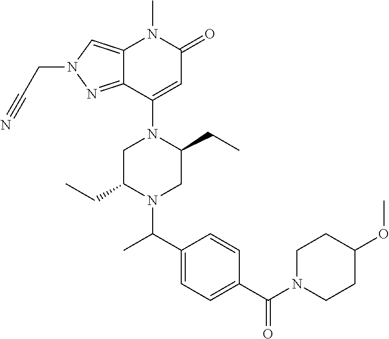 [SMILES: CC[C@H]1CN(C(C)c2ccc(C(=O)N3CCC(OC)CC3)cc2)[C@H](CC)CN1c1cc(=O)n(C)c2cn(CC#N)nc12]

### Step A: 1-(4-(4-methoxypiperidine-1-carbonyl)phenyl)ethan-1-one

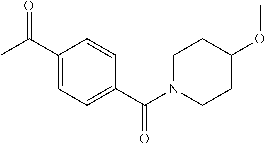 [SMILES: COC1CCN(C(=O)c2ccc(C(C)=O)cc2)CC1]

To a solution of 4-acetylbenzoic acid (164 mg, 1 mmol), 4-methoxypiperidine (140 mg, 1,2 mmol) and HATU (570 mg, 1.5 mmol) in DCM (10 mL) was added DIEA (258 mg, 2 mmol). The reaction mixture was stirred at 25° C. overnight. The mixture was diluted with water, extracted with DCM (60 mL), washed with brine, dried over Na₂SO₄, filtered, and concentrated to dryness (260 mg, crude). MS: M/e 262 (M+1)⁺.

### Step B: (4-(1-hydroxyethyl)phenyl)(4-methoxypiperidin-1-yl)methanone

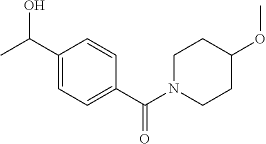 [SMILES: COC1CCN(C(=O)c2ccc(C(C)O)cc2)CC1]

To a solution of 1-(4-(4-methoxypiperidine-1-carbonyl)phenyl)ethan-1-one (260 mg, crude) in MeOH (5 mL) was added NaBH₄ (76 mg, 2 mmol) slowly at 0° C. The resulting mixture was stirred at room temperature for 1 hour. The reaction mixture was quenched with H₂O (0.5 mL) and concentrated to dryness. The residue was purified by flash column chromatography to give the desired product (150 mg, 57% for two steps). MS: M/e 264 (M+1)⁺.

### Step C: 2-(7-((2S,5R)-2,5-diethyl-4-(1-(4-(4-methoxypiperidine-1-carbonyl)phenyl)ethyl)piperazin-1-yl)-4-methyl-5-oxo-4,5-dihydro-2H-pyrazolo[4,3-b]pyridin-2-yl)acetonitrile

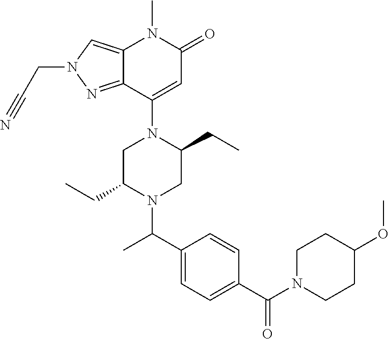 [SMILES: CC[C@H]1CN(C(C)c2ccc(C(=O)N3CCC(OC)CC3)cc2)[C@H](CC)CN1c1cc(=O)n(C)c2cn(CC#N)nc12]

To a solution of (4-(1-hydroxyethyl)phenyl)(4-methoxypiperidin-1-yl)methanone (52 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄ and filtered. The resulting filtrate was concentrated to dryness to give the titled compound(crude), which was further purified and separated into Compound A194a (6 mg) and Compound A194b (12 mg) by Prep-HPLC (Method A).

Compound A194a (the earlier isomer): ¹H NMR (400 MHz, CD₃OD) δ 7.92 (s, 1H), 7.49 (d, J=8.0 Hz, 2H), 7.40 (d, J=8.0 Hz, 2H), 5.55 (s, 1H), 5.46 (s, 2H), 4.10-3.95 (m, 1H), 3.85-3.76 (m, 1H), 3.70-3.45 (m, 4H), 3.43 (s, 3H), 3.37 (s, 3H), 3.35-3.31 (m, 2H), 3.28-3.24 (m, 1H), 3.06-2.98 (m, 1H), 2.94-2.82 (m, 1H), 2,48-2.35 (m, 1H), 2.21-1.49 (m, 8H), 1.35 (d, J=6.0 Hz, 3H), 0.96 (t, J=7.2 Hz, 3H), 0.70 (t, J=7.2 Hz, 3H) ppm. MS: M/e 574 (M+1)⁺.

Compound A194b (the later isomer): ¹H NMR (400 MHz, CD₃OD) δ 7.92 (s, 1H), 7.51 (d, J=8.0 Hz, 2H), 7.37 (d, J=8.4 Hz, 2H), 5.55 (s, 1H), 5.47 (s, 2H), 4.10-3.90 (m, 1H), 3.71-3.45 (m, 5H), 3.43 (s, 3H), 3.36 (s, 3H), 3.35-3.31 (m, 2H), 3.24-3.10 (m, 2H), 2.71-2.60 (m, 1H), 2.38-2.30 (m, 1H), 2.12-1.41 (m, 8H), 1.33 (d, J=6.8 Hz, 3H), 1.03 (t, J=7.6 Hz, 3H), 0.61 (t, J=7.6 Hz, 3H) ppm. MS: M/e 574 (M+1)⁺.

### Compound A195: 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-N-(2-methoxyethyl)-N-methylbenzamide

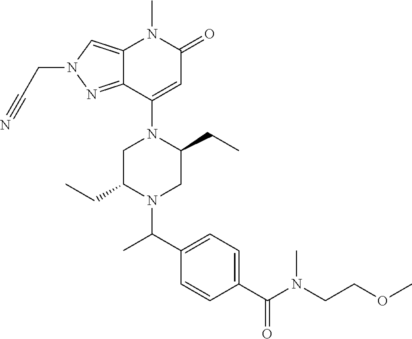 [SMILES: CC[C@H]1CN(C(C)c2ccc(C(=O)N(C)CCOC)cc2)[C@H](CC)CN1c1cc(=O)n(C)c2cn(CC#N)nc12]

### Step A: 4-acetyl-N-(2-methoxyethyl)-N-methylbenzamide

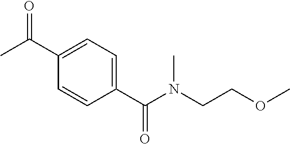 [SMILES: COCCN(C)C(=O)c1ccc(C(C)=O)cc1]

To a solution of 4-acetylbenzoic acid (164 mg, 1 mmol), 2-methoxy-N-methylethan-1-amine (107 mg, 1,2 mmol) and HATU (456 mg, 1,2 mmol) in DCM (10 mL) was added DIEA (258 mg, 2 mmol). The reaction mixture was stirred at 25° C. overnight. The mixture was diluted with water, extracted with DCM (80 mL), washed with brine, dried over Na₂SO₄, filtered, and concentrated to dryness (400 mg, crude). MS: M/e 236 (M+1)”.

### Step B: 4-(1-hydroxyethyl)N-(2-methoxyethyl)-N-methylbenzamide

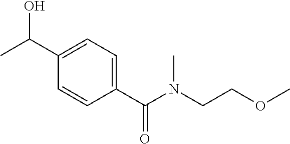 [SMILES: COCCN(C)C(=O)c1ccc(C(C)O)cc1]

To a solution of 4-acetyl-N-(2-methoxyethyl)-N-methylbenzamide (400 mg, crude) in MeOH (5 mL) was added NaBH₄ (76 mg, 2 mmol) slowly at 0° C. The resulting mixture was stirred at room temperature for 1 hour. The reaction mixture was quenched with H₂O (0.5 mL) and concentrated to dryness. The residue was purified by flash column chromatography to give the desired product (180 mg, 75% for two steps). ¹H NMR (400 MHz, DMSO-d₆) δ 7.38 (d, J=8.0 Hz, 2H), 7.31 (d, J=7.6 Hz, 2H), 5.22 (d, J=3.6 Hz, 1H), 4.78-4.69 (m, 1H), 3.65-3.35 (m, 4H), 3.31-3.14 (m, 3H), 2.95 (s, 3H), 1.32 (d, J=6.4 Hz, 3H) ppm.

### Step C: 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl-N-(2-methoxyethyl)-N-methylbenzamide

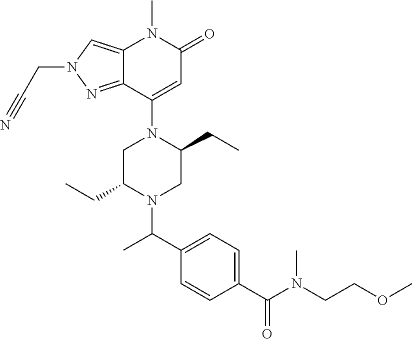 [SMILES: CC[C@H]1CN(C(C)c2ccc(C(=O)N(C)CCOC)cc2)[C@H](CC)CN1c1cc(=O)n(C)c2cn(CC#N)nc12]

To a solution of 4-(1-hydroxyethyl)-N-(2-methoxyethyl)-N-methylbenzamide (46 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄ and filtered. The resulting filtrate was concentrated to dryness to give the titled compound(crude), which was further purified and separated into Compound A195a (7 mg) and Compound A195b (12 mg) by Prep-HPLC (Method A).

Compound A195a (the earlier isomer): ¹H NMR (400 MHz, CD₃OD) δ 7.92 (s, 1H), 7.52-7.44 (m, 2H), 7.41 (d, J=8.0 Hz, 2H), 5.56 (s, 1H), 5.46 (s, 2H), 3.90-3.60 (m, 3H), 3.54-3.38 (m, 6H), 3.35-3.31 (m, 2H), 3.29-3.24 (m, 2H), 3.15-2.98 (m, 4H), 2.94-2.84 (m, 1H), 2,50-2.35 (m, 1H), 2.17-2.05 (m, 1H), 1.89-1.75 (m, 1H), 1.62-1.46 (m, 2H), 1.37 (d, J=6.4 Hz, 3H), 0.96 (t, J=7.2 Hz, 3H), 0.69 (t, J=7.2 Hz, 3H) ppm. MS: We 548 (M+1)⁺.

Compound A195b (the later isomer): ¹H NMR (400 MHz, CD₃OD) δ 7.92 (s, 1H), 7.56-7.44 (m, 2H), 7.38 (d, J=8.4 Hz, 2H), 5.55 (s, 1H), 5.47 (s, 2H), 3.76-3.52 (m, 3H), 3.53-3.36 (m, 8H), 3.35-3.31 (m, 2H), 3.28-2.98 (m, 5H), 2.71-2,58 (m, 1H), 2,41-2.30 (m, 1H), 2.05-1.90 (m, 1H), 1.75-1.45 (m, 3H), 1.32 (d, J=6.8 Hz, 3H), 1.03 (t, J=7.2 Hz, 3H), 0.62 (t, J=7.6 Hz, 3H) ppm. MS: M/e 548 (M+1)⁺.

### Compound A196: 2-(7-((2S,5R)-4-(1-(2,3-dihydrofuro[2,3-c]pyridin-5-yl)ethyl)-2,5-diethylpiperazin-1-yl)-4-methyl-5-oxo-4,5-dihydro-2H-pyrazolo[4,3-b]pyridin-2-yl)acetonitrile

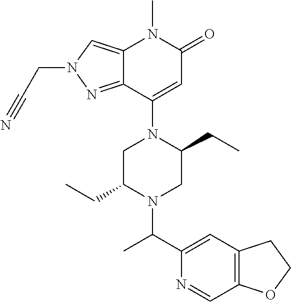 [SMILES: CC[C@H]1CN(C(C)c2cc3c(cn2)OCC3)[C@H](CC)CN1c1cc(=O)n(C)c2cn(CC#N)nc12]

### Step A: 1-(23-dihydrofuro[2.3-c]pyridin-5-yl)ethan-1-one

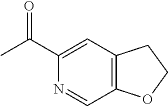

A mixture of 5-chloro-2,3-dihydrofuro[2,3-c]pyridine (1.0 g, 6.4 mmol), tributyl(1-ethoxyvinyl)stannane (3.6 g, 10.0 mmol) and Pd(PPh₀)Cl₂ (225 mg, 0.32 mmol) in toluene (10 mL) was stirred at 100° C. under N₂ for 16 hrs. The mixture was cooled and concentrated to dryness. The resulted residue was purified by column chromatography to give the titled compound (120 mg, 11%). ¹H NMR (400 MHz, CDCl₃) δ 8.16 (s, 1H), 7.99 (s, 1H), 4.71 (t, J=8.8 Hz, 2H), 3.29 (t, J=8.8 Hz, 2H), 2.67 (s, 3H). MS: M/e 164 (M+1)⁺.

### Step B: 1-(2.3-dihydrofuro[2,3-c]pyridin-5-yl)ethan-1-ol

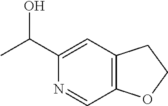

To a solution of 1-(2,3-dihydrofuro[2,3-c]pyridin-5-yl)ethan-1-one (120 mg, 0.73 mmol) in MeOH (3 mL) was added NaBH₄ (28 mg, 0.73 mmol) at room temperature and the mixture was stirred for 2 hours. The mixture was concentrated and the residue was diluted with EtOAc (10 mL). The solution was washed with brine (5 mL×2), dried over Na₂SO₄ and concentrated to dryness. The resulted residue was purified by column chromatography to give the title compound (118 g, 97%). ¹H NMR (400 MHz, DMSO-d6) δ 7.95 (s, 1H), 7.40 (s, 1H), 5.20 (d, J=4.8 Hz, 1H), 4.73-4.61 (m, 1H), 4.55 (t, J=8.8 Hz, 2H), 3.22 (t, J=8.8 Hz, 2H), 1.31 (d, J=6.4 Hz, 3H).

### Step C: 2-(7-((2S,5R)-4-(1-2,3-dihydrofuro[2,3-c]pyridin-5-yl)ethyl)-2,5-diethylpiperazin-1-yl-4-methyl-5-oxo-4,5-dihydro-2H-pyrazolo[4,3-b]pyridin-2-yl)acetonitrile

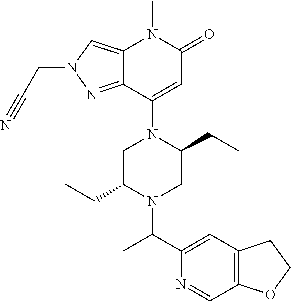 [SMILES: CC[C@H]1CN(C(C)c2cc3c(cn2)OCC3)[C@H](CC)CN1c1cc(=O)n(C)c2cn(CC#N)nc12]

To a mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol), 1-(2,3-dihydrofuro[2,3-c]pyridin-5-yl)ethan-1-ol (63 mg, 0.38 mmol) and (cyanomethyl)trimethylphosphonium iodide (127 mg, 0.53 mmol) in CH₃CN (1 mL) was added DIPEA (129 mg, 1.0 mmol). The mixture was stirred at 100° C. in a sealed tube for 16 hours. The mixture was cooled and diluted with EtOAc (20 mL), washed with brine (10 mL×3), dried over Na₂SO₄, filtered and concentrated under reduced pressure. The resulting residue was purified by flash column chromatography and Prep-HPLC (Method A) to give the titled compound (15 mg, 21%). ¹H NMR (400 MHz, CD₃OD) δ 7.95-7.86 (m, 2H), 7.58-7.47 (m, 1H), 5.56 (s, 1H), 5.50-5.43 (m, 2H), 4.67-4.58 (m, 2H), 3.93-3.69 (m, 1H), 3.57-3.46 (m, 1H), 3.43 (s, 3H), 3.37-3.31 (m, 2H), 3.30-3.19 (m, 2H), 3.18-2.97 (m, 1H), 2.93-2.62 (m, 1H), 2,45-2.20 (m, 1H), 2.13-1.49 (m, 4H), 1.36-1,29 (m, 3H), 1.07-0.92 (m, 3H), 0.77-0.63 (m, 3H). MS: M/e 476 (M+1)⁺.

### Compound A197: 2-(7-((2S,5R)-4-(1-(2,2-difluorobenzo[d][1,3]dioxol-5-yl)ethyl)-2,5-diethylpiperazin-1-yl)-4-methyl-5-oxo-4,5-dihydro-2H-pyrazolo[4,3-b]pyridin-2-yl)acetonitrile

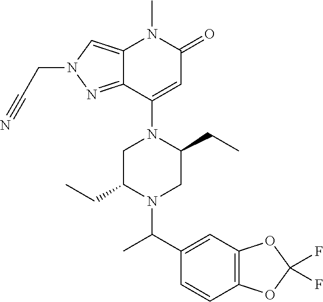 [SMILES: CC[C@H]1CN(C(C)c2ccc3c(c2)OC(F)(F)O3)[C@H](CC)CN1c1cc(=O)n(C)c2cn(CC#N)nc12]

To a mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol), 1-(2,2-difluorobenzo[d][1,3]dioxol-5-yl)ethan-1-ol (63 mg, 0.31 mmol) and (cyanomethyl)trimethylphosphonium iodide (127 mg, 0.53 mmol) in CH₃CN (1 mL) was added DIPEA (129 mg, 1.0 mmol). The mixture was stirred at 100° C. in a sealed tube for 16 hours. The mixture was cooled and diluted with EtOAc (10 mL), washed with brine (5 mL×3), dried over Na₂SO₄, filtered and concentrated under reduced pressure. The resulting residue was purified by flash column chromatography to give the titled compound (32 mg, 41%). ¹H NMR (400 MHz, CD₃OD) δ 7.95-7.87 (m, 1H), 7.35-7.24 (m, 1H), 7.22-7.07 (m, 2H), 5.60-5.52 (m, 1H), 5.49-5.42 (m, 2H), 3.88-3.57 (m, 1H), 3.56-3.38 (m, 4H), 3.30-3.23 (m, 2H), 3.19-2.95 (m, 1H), 2.91-2,59 (m, 1H), 2,51-2.30 (m, 1H), 2.16-1.49 (m, 4H), 1.38-1,27 (m, 3H), 1.07-0.90 (m, 3H), 0.78-0.57 (m, 3H). MS: M/e 513 (M+1)⁺.

### Compound A198: 2-(7-((2S,5R)-2,5-diethyl-4-(1-(4-((methylsulfonyl)methyl)phenyl)ethyl)piperazin-1-yl)-4-methyl-5-oxo-4,5-dihydro-2H-pyrazolo[4,3-b]pyridin-2-yl)acetonitrile

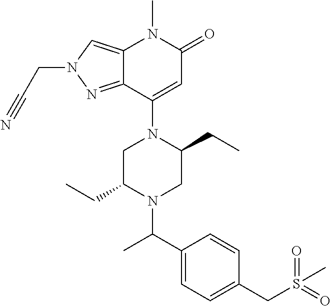 [SMILES: CC[C@H]1CN(C(C)c2ccc(CS(C)(=O)=O)cc2)[C@H](CC)CN1c1cc(=O)n(C)c2cn(CC#N)nc12]

### Step A: 1-(4-(bromomethyl)phenyl)ethan-1-one

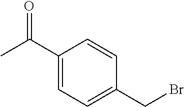

To a solution of 1-(p-tolyl)ethan-1-one (1.34 g, 10 mmol) and NBS (1.96 g, 11 mmol) in CH₃CN (20 mL) was added AIBN (50 mg). The reaction mixture was stirred at 95° C. under N₂ overnight. The mixture was cooled to room temperature, diluted with MTBE (20 mL), filtered, and the filtrate was concentrated to dryness. The resulting residue was purified by flash column chromatography to give the titled compound (1.7 g, 79%).

### Step B: 1-(4-(methylsulfonyl)methyl)thenyl)ethan-1-one

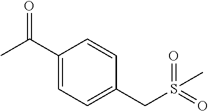

A solution of 1-(4-(bromomethyl)phenyl)ethan-1-one (212 mg, 1 mmol) and sodium methanesulfinate (204 mg, 2 mmol) in EtOH (3 mL) was stirred at 100° C. in a sealed bottle overnight. The mixture was cooled to room temperature and concentrated to dryness. The residue was purified by flash column chromatography to give the titled compound (180 mg, 83%). MS: M/e 213 (M+1)⁺.

### Step C: 1-(4-((methylsulfonyl)methyl)phenyl)ethan-1-ol

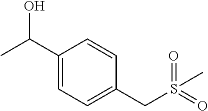

To a solution of 1-(4-((methylsulfonyl)methyl)phenyl)ethan-1-one (180 mg, 0.85 mmol) in MeOH (5 mL) was added NaBH₄ (32 mg. 0.85 mmol) slowly at 0° C. The resulting mixture was stirred at room temperature for 1 hour. The reaction mixture was quenched with H₂O (10 mL) and extracted with DCM (80 mL), washed with brine, dried over Na₂SO₄, filtered, and concentrated to dryness. The resulting residue was purified by flash column chromatography to give the titled product (140 mg, 75%). ¹H NMR (400 MHz, DMSO-d₆) δ 7.40-7.30 (m, 4H), 5.18 (d, J=4.0 Hz, 1H), 4.77-4.68 (m, 1H), 4.44 (s, 2H), 2.88 (s, 3H), 1.31 (d, J=6.4 Hz, 3H) ppm.

### Step D: 2-(7-((2S,5R)-2,5-diethyl-4-(1-(4-((methylsulfonyl)methyl)phenyl)ethyl)piperazin-1-yl)-4-methyl-5-oxo-4,5-dihydro-2H-pyrazolo[4,3-b]pyridin-2-yl)acetonitrile

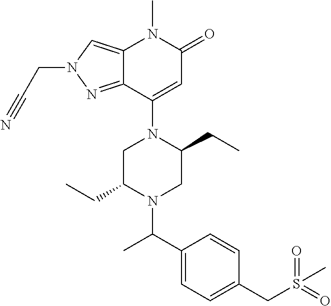 [SMILES: CC[C@H]1CN(C(C)c2ccc(CS(C)(=O)=O)cc2)[C@H](CC)CN1c1cc(=O)n(C)c2cn(CC#N)nc12]

To a solution of 1-(4-((methylsulfonyl)methyl)phenyl)ethan-1-ol (42 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile yl)acetonitrile (33 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered and concentrated to dryness. The resulting residue was purified by Prep-HPLC (Method A) to give the titled compound (9 mg, 16%). ¹H NMR (400 MHz, CD₃OD) δ 7.93-7.89 (m, 1H), 7.50-7.39 (m, 4H), 5.55 (s, 1H), 5.46 (s, 2H), 4.44-4.38 (m, 2H), 3.82-3.49 (m, 2H), 3.43 (s, 3H), 3.35-3.31 (m, 1H), 3.27-2.98 (m, 2H), 2.92-2.80 (m, 3.5H), 2.70-2,58 (m, 0.5H), 2,48-2.30 (m, 1H), 2.19-1.48 (m, 4H), 1.39-1,28 (m, 3H), 1.06-0.89 (m, 3H), 0.75-0.55 (m, 3H) ppm. MS: M/e 525 (M+1)⁺.

### Compound A199: 2-(7-((2S,5R)-4-(1-(6-(1,1-difluoroethyl)pyridin-3-yl)ethyl)-2,5-diethylpiperazin-1-yl)-4-methyl-5-oxo-4,5-dihydro-2H-pyrazolo[4,3-b]pyridin-2-yl)acetonitrile

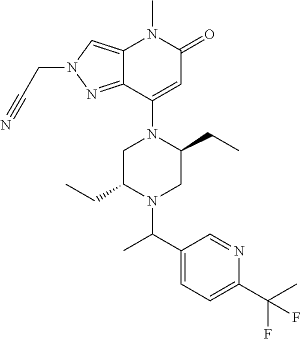 [SMILES: CC[C@H]1CN(C(C)c2ccc(C(C)(F)F)nc2)[C@H](CC)CN1c1cc(=O)n(C)c2cn(CC#N)nc12]

### Step A: 6-(1,1-difluoroethyl)nicotinonitrile

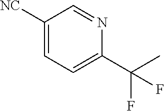

To a stirred solution of 6-acetylnicotinonitrile (390 mg, 2.67 mmol) in CH₂Cl₂ (15 mL) was added a solution of DAST (1.72 g, 10.68 mmol) at 0° C. After then, the reaction mixture was stirred for 3 days. More DAST (0.86 g, 5.34 mmol) was added and stirred for another day. The reaction was quenched with aq. NaHCO₃, extracted with CH₂Cl₂ (10 mL×3). The combined organic layers were washed with brine, dried over Na₂SO₄ and concentrated. The resulting residue was purified by flash column chromatography to give the titled compound (300 mg, 66.7%). ¹H NMR (400 MHz, CD₃OD) δ 8.98 (s, 1H), 8.33 (dd, J=8.2, 1.6 Hz, 1H), 7.87 (d, .=8.2 Hz, 1H), 2.00 (t, J=18.8 Hz, 3H) ppm.

### Step B: 1-4641.1-difluoroethyl)pyridin-3-yl)ethan-1-one

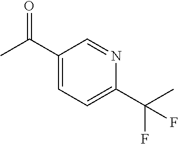

To a stirred solution of 6-(1,1-difluoroethyl)nicotinonitrile (100 mg, 0.6 mmol) in THF (5 mL) was added MeMgBr (0.4 mL, 3.0 M, 1,2 mmol) dropwise at 0° C. And MeMgBr (0.4 mL) was added several times until the reaction was almost complete. The reaction was quenched with aq·NH₄Cl, extracted with EtOAc (10 mL×3). The combined organic layers were washed with brine, dried over Na₂SO₄ and concentrated. The resulting residue was purified by Pre-TLC (petroleum ether/EtOAc=5:1) to give the titled compound (40 mg, 36%). MS: M/e 186 (M+1)⁺.

### Step C: 1-(6-(1.1-difluoroethyl)pyridin-3-yl)ethan-1-ol

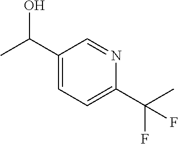

To a stirred solution of 1-(6-(1,1-difluoroethyl)pyridin-3-yl)ethan-1-one (40 mg, 0.22 mmol) in MeOH (5 mL) was added NaBH₄ (8.2 mg, 0.22 mmol). After stirred for 10 min, the reaction mixture was concentrated, then treated with H₂O, extracted with EtOAc (10 mL×2). The combined organic layers were washed with brine, dried over Na₂SO₄ and concentrated to give the titled compound (28 mg, 68%). MS: M/e 188 (M+1)⁺.

### Step D: 2-(7-((2S,5R)-4-(1-(6-(1.1-difluoroethyl)pyridin-3-yl)ethyl)-2,5-diethylpiperazin-1-yl-4-methyl-5-oxo-4,5-dihydro-2H-pyrazolo[4,3-b]pyridin-2-yl)acetonitrile

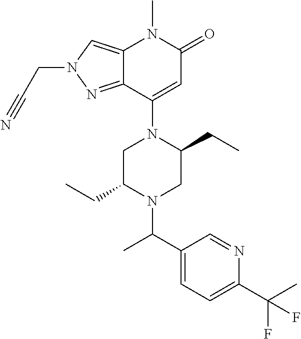 [SMILES: CC[C@H]1CN(C(C)c2ccc(C(C)(F)F)nc2)[C@H](CC)CN1c1cc(=O)n(C)c2cn(CC#N)nc12]

A mixture of 2-(7-((2S,5R)-2,5-diethylpiperazin-1-yl)-4-methyl-5-oxo-4,5-dihydro-2H-pyrazolo[4,3-b]pyridin-2-yl)acetonitrile (32.8 mg, 0.1 mmol), 1-(6-(1,1-difluoroethyl)pyridin-3-yl)ethan-1-ol (28 mg, 0.15 mmol), (cyanomethyl)trimethylphosphonium iodide (72.9 mg, 0.3 mmol) and DIPEA (129 mg, 1 mmol) in CH₃CN (4 mL) was stirred overnight at 100° C. in a sealed tube. The reaction mixture was diluted with EtOAc (15 mL), washed with H₂O, brine, dried over Na₂SO₄, concentrated to dryness. The resulting residue was purified by Prep-HPLC (Method B) to give the titled compound (13 mg). ¹H NMR (400 MHz, CD₃OD) δ 8.62 (d, J=12,4 Hz, 1H), 8.03-7.96 (m, 1H), 7.93 (s, 1H), 7.73-7.66 (m, 1H), 5.56 (d, J=2.8 Hz, 1H), 5.47 (d, J=3.2 Hz, 2H), 3.97-3.73 (m, 1H), 3.55-3.48 (m, 0.5H), 3.43 (s, 3H), 3.34-3.28 (m, 2,5 H), 3.21-2.67 (m, 2H), 2,42-2.25 (m, 1H), 2.05-1.92 (m, 3H), 2.16-1.51 (m, 4H), 1.43-1.33 (m, 3H), 1.07-0.92 (m, 3H), 0.78-0.58 (m, 3H) ppm. MS: M/e 498 (M+1)⁺.

### Compound A200

2-(but-2-yn-1-yl)-7-((2S,5R)-4-(1-(2,3-dihydrofuro[2,3-b]pyridin-6-yl)ethyl)-5-ethyl-2-methylpiperazin-1-yl)-4-methyl-2,4-dihydro-5H-pyrazolo[4,3-b]pyridin-5-one

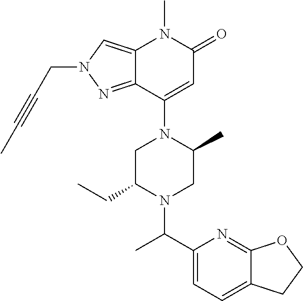 [SMILES: CC#CCn1cc2c(n1)c(N1C[C@@H](CC)N(C(C)c3ccc4c(n3)OCC4)C[C@@H]1C)cc(=O)n2C]

To a solution of 2-(but-2-yn-1-yl)-7-((2S,5R)-5-ethyl-2-methylpiperazin-1-yl)-4-methyl-2,4-dihydro-5H-pyrazolo[4,3-b]pyridin-5-one (20 mg, 0.06 mmol) in CH₃CN (1 mL) and was added 1-(2,3-dihydrofuro[2,3-b]pyridin-6-yl)ethan-1-ol (20 mg, 0.12 mmol), (cyanomethyl)trimethylphosphonium iodide (60 mg, 0.18 mmol) and DIPEA(78 mg, 0.6 mmol).The resulting mixture was stirred at 105° C. overnight. The reaction solvent was removed under reduce pressure and purified by flash column chromatography (DCM:MeOH) and Prep-HPLC (Method A) to give the titled compound(10 mg). ¹H NMR (400 MHz, CD₃OD) δ 7.91 (d, J=5.1 Hz, 1H), 7.67-7.58 (m, 1H), 7.04 (dd, J=19.2, 7.4 Hz, 1H), 5.55 (d, J=4.2 Hz, 1H), 5.04 (dd, J=5.9, 2,5 Hz, 2H), 4.74-4.30 (m, 3H), 3.99-3.71 (m, 1H), 3.58 (d, J=13.5 Hz, 1H), 3.45 (s, 3H), 3.41-3.34 (m, 1H), 3.28-2.80 (m, 4H), 2.72-2.36 (m, 1H), 1.85 (d, J=2.6 Hz, 3H), 1.81-1.48 (m, 2H), 1.43-1,23 (m, 6H),1.10-0.75 (m, 3H) ppm. MS: M/e 475 (M+1)⁺.

### Compound A201: 2-(7-((2S,5R)-4-(1-(5-cyclopropoxypyridin-2-yl)ethyl)-2,5-diethylpiperazin-1-yl)-4-methyl-5-oxo-4,5-dihydro-2H-pyrazolo[4,3-b]pyridin-2-yl)acetonitrile

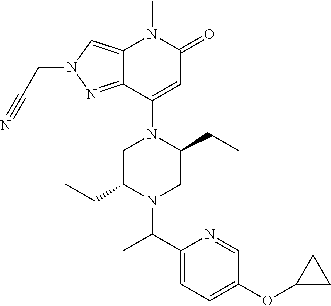 [SMILES: CC[C@H]1CN(C(C)c2ccc(OC3CC3)cn2)[C@H](CC)CN1c1cc(=O)n(C)c2cn(CC#N)nc12]

### Step A: 5-cyclopropoxypicolinonitrile

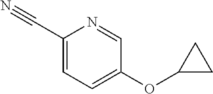 [SMILES: N#Cc1ccc(OC2CC2)cn1]

A mixture of 5-fluoropicolinonitrile (500 mg, 4.1 mmol) in DMF (10 mL) was added cyclopropanol (476 mg, 8.2 mol) and Cs₂CO₃ (2.6 g, 8.2 mol). The reaction mixture was stirred at room temperature for 3 hours. The resulting mixture was diluted with ethyl acetate and washed with water. Then the resulting organic layer was concentrated and purified by flash column chromatography to give the titled compound (400 mg, 61%). MS: M/e 161 (M+1)⁺.

### Step B: 1-(5-cyclopropoxypyridin-2-yl)ethan-1-one

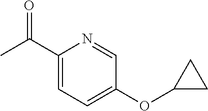

To a solution of 5-cyclopropoxypicolinonitrile (400 mg, 2,5 mmol) in THF (5 mL) was added methyl magnesium bromide (0.76 mL, 3M, 2.28 mol) at 0° C. and the resulting mixture was stirred at room temperature for 2 hours. The reaction was quenched by adding aqueous NH₄Cl. The resulting mixture was extracted with ethyl acetate. Then the resulting organic layer was concentrated to give the titled compound (350 mg). MS: M/e 178 (M+1)⁺.-

### Step C: 1-(5-cyclopropoxypyridin-2-yl)ethan-1-ol

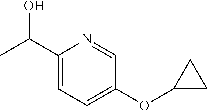

To a solution of 1-(5-cyclopropoxypyridin-2-yl)ethan-1-one (350 mg, 1.9 mmol) in MeOH (3 mL) was added NaBH₄ (50 mg, 1.3 mmol) at room temperature and the resulting mixture was stirred at room temperature for 5 min. The reaction mixture was diluted with DCM and washed with water, dried over Na₂SO₄ and concentrated and purified by flash column chromatography to give the titled compound (160 mg, two steps, 36%). MS: M/e 180 (M+1)⁺.

### Step D: 2-(7-((2S,5R)-4-(1-(5-cyclopropoxypyridin-2-yl)ethyl)-2,5-diethylpiperazin-1-yl-4-methyl-5-oxo-4,5-dihydro-2H-pyrazolo[4,3-b]pyridin-2-yl)acetonitrile

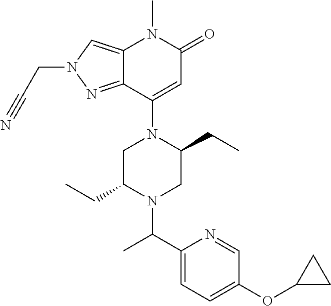 [SMILES: CC[C@H]1CN(C(C)c2ccc(OC3CC3)cn2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (2 mL) and was added 1-(5-cyclopropoxypyridin-2-yl)ethan-1-ol (55 mg, 0.3 mmol), (cyanomethyl)trimethylphosphonium iodide (111 mg, 0.45 mmol) and DIPEA(197 mg, 1.5 mmol).The resulting mixture was stirred at 105° C. overnight. The reaction solvent was removed under reduce pressure. The resulting residue was purified by flash column chromatography and Prep-HPLC (Method A) to give the titled compound (18 mg). ¹H NMR (400 MHz, CD₃OD) δ 8.26 (d, J=2,4 Hz, 1H), 7.93 (d, J=4.1 Hz, 1H), 7.63-7.47 (m, 2H), 5.57 (d, J=4.0 Hz, 1H), 5.47 (d, J=3.9 Hz, 2H), 4.78-4.10 (m, 2H), 4.08-3.80 (m, 2H), 3.56 (d, J=13.3 Hz, 1H), 3.44 (s, 3H), 3.29-2.81 (m, 2H), 2.61-2.31 (m, 1H), 2.17-1.50 (m, 4H), 1.39 (dd, J=8.4, 5.0 Hz, 3H), 1.00 (dt, J=34.5, 7.3 Hz, 3H), 0.91-0.64 (m, 7H) ppm. MS: M/e 490 (M+1)”.

### Compound A202:2-(7-((2S,5R)-2,5-diethyl-4-(1-(4-(3-methoxypyrrolidine-1-carbonyl)phenyl)ethyl)piperazin-1-yl)-4-methyl-5-oxo-4,5-dihydro-2H-pyrazolo[4,3-b]pyridin-2-yl)acetonitrile

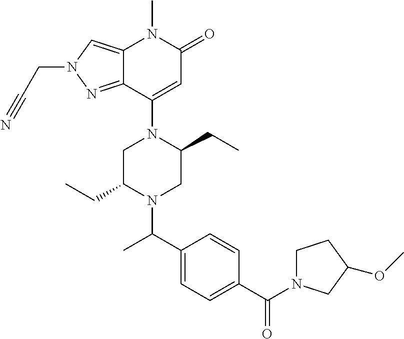 [SMILES: CC[C@H]1CN(C(C)c2ccc(C(=O)N3CCC(OC)C3)cc2)[C@H](CC)CN1c1cc(=O)n(C)c2cn(CC#N)nc12]

### Step A: 1-(4-(3-methylnortropidine-1-carbonyl)phenylethan-1-one

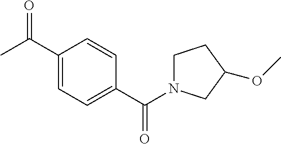 [SMILES: COC1CCN(C(=O)c2ccc(C(C)=O)cc2)C1]

To a solution of 4-acetylbenzoic acid (164 mg, 1 mmol), 3-methoxypyrrolidine (120 mg, 1,2 mmol) and HATU (456 mg, 1,2 mmol) in DCM (10 mL) was added DIEA (190 mg, 1.5 mmol). The reaction mixture was stirred at 25° C. overnight. The mixture was diluted with water, extracted with DCM (70 mL), washed with brine, dried over Na₂SO₄, filtered, and concentrated to dryness (300 mg, crude). MS: We 248 (M+1)⁺.

### Step B: (4-(1-hydroxyethyl)phenyl)(3-methoxypyrrolidin-1-yl)methanone

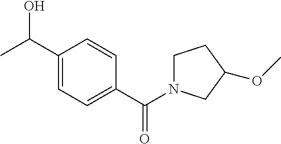 [SMILES: COC1CCN(C(=O)c2ccc(C(C)O)cc2)C1]

To a solution of 1-(4-(3-methoxypyrrolidine-1-carbonyl)phenyl)ethan-1-one (300 mg, crude) in MeOH (5 mL) was added NaBH₄ (76 mg, 2 mmol) slowly at 0° C. The resulting mixture was stirred at room temperature for 1 hour. The reaction mixture was quenched with H₂O (0.5 mL) and concentrated to dryness. The residue was purified by flash column chromatography to give the desired product (160 mg, 64% for two steps). ¹H NMR (400 MHz, DMSO-d₆) δ 7.46 (d, J=8.0 Hz, 2H), 7.39 (d, J=8.0 Hz, 2H), 5.24 (d, J=4.4 Hz, 1H), 4.80-4.70 (m, 1H), 4.02-3.89 (m, 1H), 3.65-3.38 (m, 4H), 3.26 (s, 1.5H), 3.15 (s, 1.5H), 2.02-1.85 (m, 2H), 1.32 (d, J=6.8 Hz, 3H) ppm.

### Step C: 2-(7-((2S,5R)-2,5-diethyl-4-(1-(4-(3-methoxypyrrolidine-1-carbonyl)phenylethyl)piperazin-1-yl)-4-methyl-5-oxo-4,5-dihydro-2H-pyrazolo[4,3-b]pyridin-2-yl)acetonitrile

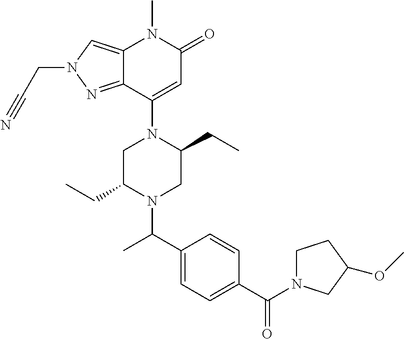 [SMILES: CC[C@H]1CN(C(C)c2ccc(C(=O)N3CCC(OC)C3)cc2)[C@H](CC)CN1c1cc(=O)n(C)c2cn(CC#N)nc12]

To a solution of (4-(1-hydroxyethyl)phenyl)(3-methoxypyrrolidin-1-yl)methanone (50 mg, 0.2 mmol), 2-(7-((2S,5R)-2,5-diethylpiperazin-1-yl)-4-methyl-5-oxo-4,5-dihydro-2H-pyrazolo[4,3-b]pyridin-2-yl)acetonitrile (33 mg, 0.1 mmol) and (cyanomethyl) trimethyl phosphonium iodide (73 mg, 0.3 mmol) in CH₃CN (1 mL) was added DIPEA (65 mg, 0.5 mmol). The mixture was sealed in a bottle and heated at 100° C. for 16 hours. The mixture was cooled to room temperature, diluted with water, extracted with EA (60 mL), washed with brine, dried over Na₂SO₄, filtered, and concentrated to dryness. The resulting residue was purified by Prep-HPLC (Method A) to give the titled compound(15 mg, 26%). ¹H NMR (400 MHz, CD₃OD) δ 7.92 (s, 1H), 7.56-7.46 (m, 4H), 5.56 (s, 1H), 5.47 (s, 2H), 4.20-3.80 (m, 2H), 3.76-3.45 (m, 5H), 3.43 (s, 3H), 3.39-3.35 (m, 1H), 3.35-3.31 (m, 2H), 3.28-3.23 (m, 2H), 3.22-3.05 (m, 1H), 2.98-2.84 (m, 0.5H), 2.74-2.62 (m, 0.5H), 2,55-2.34 (m, 1H), 2.21-1.49 (m, 6H), 1.43-1.31 (m, 3H), 1.06-0.95 (m, 3H), 0.75-0.59 (m, 3H) ppm. MS: M/e 560 (M+1)⁺.

### Compound A203: 2-(7-((2S,5R)-2,5-diethyl-4-(1-(4-((3-methoxypyrrolidin-1-yl)methyl)phenyl)ethyl)piperazin-1-yl)-4-methyl-5-oxo-4,5-dihydro-2H-pyrazolo[4,3-b]pyridin-2-yl)acetonitrile

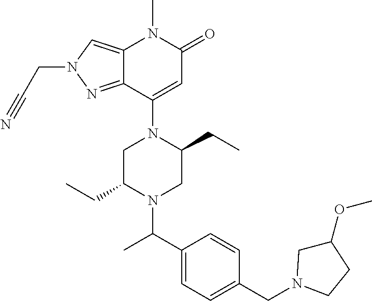 [SMILES: CC[C@H]1CN(C(C)c2ccc(CN3CCC(OC)C3)cc2)[C@H](CC)CN1c1cc(=O)n(C)c2cn(CC#N)nc12]

### Step A: 1-(4-bromobenzyl)-3-methoxypyrrolidine

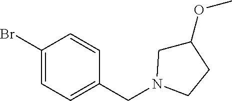

A solution of 4-bromobenzaldehyde (500 mg, 2.70 mmol), 3-methoxypyrrolidine (300 mg, 2.97 mmol) and STAB (1.15 g, 5.42 mmol) in DCM (10 ml) was stirred at RT overnight. The solution was washed with aq. NaHCO₃ (10 ml×2), dried over Na₂SO₄ and concentrated to dryness. The resulting residue was purified by flash column chromatography with 0-10% MeOH in DCM to give the titled compound (720 mg, 98%). MS: M/e 270,272 (M+1)⁺.

### Step B: 1-(4-((3-methoxypyrrolidin-1-yl)methyl)phenyl))ethan-1-one

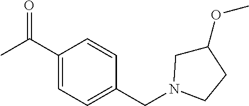 [SMILES: COC1CCN(Cc2ccc(C(C)=O)cc2)C1]

A solution of 1-(4-bromobenzyl)-3-methoxypyrrolidine (360 mg, 1.33 mmol), ethyl tributylstannanecarboxylate (722 mg, 2 mmol) and Pd(PPh₃)₂Cl₂ (47 mg, 0.067 mmol) in toluene(6 ml) was stirred at 100° C. overnight. The solution was cooled to RT. HCl/1,4-dioxane (4M, 2 ml) was added and stirred at RT for 10 min. The solution was diluted with EtOAc (15 ml), washed with aq. Na₂CO₃ (10 ml×2), dried over Na₂SO₄ and concentrated to dryness. The resulting residue was purified by flash column chromatography with 0-10% MeOH in DCM to give the titled compound (120 mg, 38%). MS: M/e 234 (M+1)⁺.

### Step C: 1-(4-((3-methoxyovrrolidin-1-yl)methyl)phenyl)ethan-1-ol

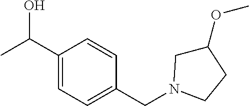 [SMILES: COC1CCN(Cc2ccc(C(C)O)cc2)C1]

A solution of 1-(4-((3-methoxypyrrolidin-1-yl)methyl)phenyl)ethan-1-one (120 mg, 0.52 mmol) and NaBH₄ (20 mg, 0.52 mmol) in MeOH (2 ml) was stirred at RT for 40 min. The solution was diluted with EtOAc(10 ml), washed with brine (10 ml×2), dried over Na₂SO₄ and concentrated to dryness to give the titled compound (120 mg, 99%). MS: M/e 236 (M+1)⁺.

### Step D: 2-(7-((2S,5R)-2,5-diethyl-4-(1-(4-((3-methoxypyrrolidin-1-yl)methyl)phenyl)ethyl)piperazin-1-yl)-4-methyl-5-oxo-4,5-dihydro-2H-pyrazolo[4,3-b]pyridin-2-yl)acetonitrile

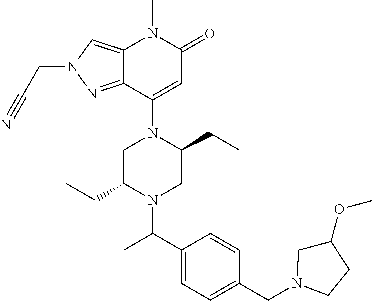 [SMILES: CC[C@H]1CN(C(C)c2ccc(CN3CCC(OC)C3)cc2)[C@H](CC)CN1c1cc(=O)n(C)c2cn(CC#N)nc12]

A solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (67 mg, 0.20 mmol), 1-(4-((3-methoxypyrrolidin-1-yl)methyl)phenyl)ethan-1-ol (120 mg, 0.51 mmol), (cyanomethyl)trimethylphosphonium iodide (149 mg, 0.61 mmol) and DIPEA (264 mg, 2.05 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography to give the titled compound (crude), which was further separated into Compound A203a (2,4 mg) and Compound A203b (3.3 mg) by Prep-HPLC (Method A).

Compound A203a (the earlier peak): ¹H NMR (500 MHz, DMSO-d6): δ 8.22 (s, 1.60H), 7.97 (s, 1H), 7.27 (q, J=8.1 Hz, 4H), 5.60 (s, 2H), 5.38 (s, 1H), 3.89-3.86 (m, 1H), 3.67 (q, J=6.3 Hz, 2H), 3.54 (q, J=12.9 Hz, 3H), 3.27 (s, 3H), 3.14 (s, 3H), 3.13-3.08 (m, 1H), 2.90 (d, J=11.6 Hz, 1H), 2.74 (dd, J=11.7, 3.3 Hz, 1H), 2.68-2.63 (m, 1H), 2,53 (s, 1H), 2,45-2.38 (m, 2H), 2.31 (s, 1H), 2.04-1.94 (m, 2H), 1.68-1.58 (m, 2H), 1.46-1.39 (m, 2H), 1,27 (d, J=6.4 Hz, 3H), 0.86 (t, J=7.0 Hz, 3H), 0.58 (t, J=7.3 Hz, 3H) ppm. MS: M/e 546 (M+1)⁺.

Compound A203b (the later peak): ¹H NMR (500 MHz, DMSO-d6): δ 8.18 (s, 1.5H), 7.98 (s, 1H), 7.31 (d, J=8.0 Hz, 2H), 7.24 (d, J=7.9 Hz, 2H), 5.61 (s, 2H), 5.37 (s, 1H), 3.89-3.84 (m, 1H), 3.57-3.52 (m, 3H), 3.33 (d, J=12.0 Hz, 1H), 3.27 (s, 3H), 3.14 (d, J=0.8 Hz, 3H), 3.04 (d, J=9.7 Hz, 1H), 2.65 (td, J=9.8, 6.3 Hz, 1H), 2,58-2,51 (m, 3H), 2,49-2.36 (m, 3H), 2.23 (d, J=11.9 Hz, 1H), 2.02-1.94 (m, 1H), 1.91-1.82 (m, 1H), 1.68-1.61 (m, 1H), 1.59-1.52 (m, 1H), 1.49-1.37 (m, 2H), 1,24 (d, J=6.5 Hz, 3H), 0.95 (t, J=7.2 Hz, 3H), 0.50 (t, J=7.0 Hz, 3H) ppm. MS: M/e 546 (M+1)*.

### Compound A204: 2-(7-((2S,5R)-2,5-diethyl-4-(1-(3-methylpyrazolo[1,5-a]pyridin-6-yl)ethyl)piperazin-1-yl)-4-methyl-5-oxo-4,5-dihydro-2H-pyrazolo[4,3-b]pyridin-2-yl)acetonitrile

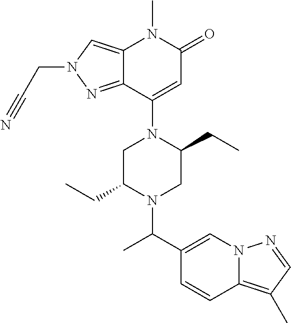 [SMILES: CC[C@H]1CN(C(C)c2ccc3c(C)cnn3c2)[C@H](CC)CN1c1cc(=O)n(C)c2cn(CC#N)nc12]

### Step A: methyl 6-bromopyrazolo[1,5-a]pyridine-3-carboxylate

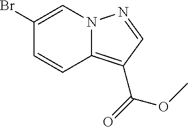

A solution of 6-bromopyrazolo[1,5-a]pyridine-3-carboxylic acid (2.6 g, 10.8 mol) in SOCl₂ (10 mL) was stirred at 70° C. for 2 h. The mixture was cooled down to rt and concentrated in vacuo to remove SOCl₂. The residue was added MeOH (10 ml) and stirred at rt for 15 mins. The mixture was concentrated in vacuo. The residue was added saturated NaHCO₃ solution (20 ml) and extracted with EA (20 mL×3), then concentrated by using a rotary evaporator to give a residue. The resulting residue was purified by flash column chromatography to give the titled compound (2.6 g, 95%). MS: M/e 255 (M+1)⁺.

### Step B: 6-bromopyrazolo[1,5-a]pyridin-3-yl)methanol

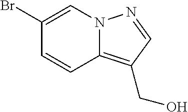 [SMILES: OCc1cnn2cc(Br)ccc12]

To a solution of methyl 6-bromopyrazolo[1,5-a]pyridine-3-carboxylate (2.6 g, 10.2 mol) in THF (20 mL) at 0° C. was slowly added LiAlH₄ (388 mg, 10.1 mol). The reaction mixture was stirred at 0° C. for 1 h. The mixture was slowly added H₂O (20 ml) at 0° C. and extracted with EA (20 mL×3). The organic phase was dried with Na₂SO₄ and concentrated by using a rotary evaporator to give a residue. The resulting residue was purified by flash column chromatography to give the titled compound (1 g, 43.4%). MS: M/e 227 (M+1)⁺.

### Step C: 6-bromo-3-methylpyrazolo[15-a]pyridine

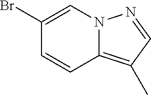

To a solution of (6-bromopyrazolo[1,5-a]pyridin-3-yl)methanol (200 mg, 0.88 mol) in TFA (2 mL) was added Et₃SiH (2 ml). The reaction mixture was stirred at 70° C. for 16 h. The mixture was concentrated in vacuo. The residue was added saturated NaHCO₃ solution (20 ml) and extracted with EA (20 mL×3), then concentrated by using a rotary evaporator to give a residue. The resulting residue was purified by flash column chromatography to give the titled compound (160 mg, 86.9%). MS: M/e 211 (M+1)⁺.

### Step D: 1-(3-methylpyrazolo[1,5-a]pyridin-6-yl)ethan-1-one

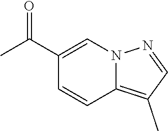

To a solution of 6-bromo-3-methylpyrazolo[1,5-a]pyridine (140 mg, 0.67 mol) in toluene (10 mL) was added Pd(PPh₃)₂Cl₂ (70 mg, 0.1 mol) and tributyl(1-ethoxyvinyl)stannane (365 mg, 1 mmol). The reaction mixture was protected by N₂ atmosphere and stirred at 90° C. for 16 h. The mixture was added 4N HCl in dioxane (0.5 ml) and stirred at rt for 15 mins. The mixture was added saturated NaHCO₃ aqueous solution (10 ml) and extracted with EA (20 mL×3). The organic phase was dried with Na₂SO₄ and concentrated by using a rotary evaporator to give a residue. The resulting residue was purified by flash column chromatography to give the titled compound (53 mg, 45.6%). MS: We 175 (M+1)⁺.

### Step E: 1-(3-methylpyrazolo[1,5-a]pyridin-6-yl)ethan-1-ol

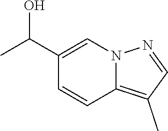

To a solution of 1-(3-methylpyrazolo[1,5-a]pyridin-6-yl)ethan-1-one (53 mg, 0.3 mol) in MeOH was added NaBH₄ (38 mg, 1 mmol). The reaction mixture was stirred at it for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (40 mg, 75.5%). MS: We 177 (M+1)⁺.

### Step F: 2-(7-((2S,5R)-2,5-diethyl-4-(1-(3-methylpyrazolo[1,5-a]pyridin-6-yl)ethyl)piperazin-1-yl)-4-methyl-5-oxo-4,5-dihydro-2H-pyrazolo[4,3-b]pyridin-2-yl)acetonitrile

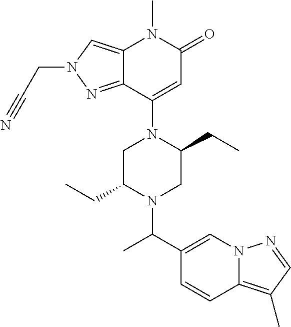 [SMILES: CC[C@H]1CN(C(C)c2ccc3c(C)cnn3c2)[C@H](CC)CN1c1cc(=O)n(C)c2cn(CC#N)nc12]

To a solution of 2-(6-((2S,5R)-2,5-dimethylpiperazin-1-yl)-9-ethyl-3-methyl-2-oxo-3,9-dihydro-2H-purin-8-yl)acetonitrile (30 mg, 0.1 mmol), 1-(3-methylpyrazolo[1,5-a]pyridin-6-yl)ethan-1-ol (40 mg, 0.22 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EA (20 mL×3). The combined organic layers were washed with brine, dried over Na₂SO₄, filtered, and concentrated to dryness. The resulting residue was purified and separated into Compound A204a (0.3 mg) and Compound A204b (1.1 mg) by Prep-HPLC (Method A).

Compound A204a (the earlier peak): ¹H NMR (500 MHz, CD₃OD) δ 8.38 (s, 1H), 7.92 (s, 1H), 7.74 (s, 1H), 7.58 (d, J=9.2 Hz, 1H), 7.30 (m, 1H), 5.56 (s, 1H), 5.46 (s, 2H), 4.58 (s, 1H), 3.81 (q, J=6.4 Hz, 1H), 3.43 (s, 3H), 3.32 (s, 1H), 3.02 (d, J=12.0 Hz, 1H), 2.90 (dd, J=12.0, 3.8 Hz, 1H), 2,51 (s, 1H), 2.32 (s, 3H), 2.12 (m, 2H), 1.83 (m, 1H), 1.58 (m, 2H), 1.42 (d, J=6.5 Hz, 3H), 0.96 (t, J=7.4 Hz, 3H), 0.74 (t, J=7.4 Hz, 3H) ppm. MS: M/e 487 (M+1)⁺.

Compound A204b (the later peak): ¹H NMR (400 MHz, CD₃OD) δ 8.40 (s, 1H), 7.92 (s, 1H), 7.73 (s, 1H), 7.55 (d, J=9.2 Hz, 1H), 7.32 (d, J=9.2 Hz, 1H), 5.56 (s, 1H), 5.47 (s, 2H), 4.58 (s, 1H), 3.67 (q, J=6.5 Hz, 1H), 3.52 (d, J=12.0 Hz, 1H), 3.43 (s, 3H), 3.15 (d, J=10.8 Hz, 1H), 2.71 (dd, J=12,4, 3.8 Hz, 1H), 2,49 (d, J=12.0 Hz, 1H), 2.31 (s, 3H), 1.96 (m, 2H), 1.69 (ddd, J=17.7, 11.6, 6.3 Hz, 2H), 1.58 (dd, J=14.3, 6.7 Hz, 1H), 1.39 (d, J=6.6 Hz, 3H), 1.03 (t, J=7.4 Hz, 3H), 0.64 (t, J=7.4 Hz, 3H) ppm. MS: M/e 507 (M+1)⁺.

### Compound A205: 2-(7-((2S,5R)-2,5-diethyl-4-(1-(4-(methoxymethyl)phenyl)ethyl)piperazin-1-yl)-4-methyl-5-oxo-4,5-dihydro-2H-pyrazolo[4,3-b]pyridin-2-yl)acetonitrile

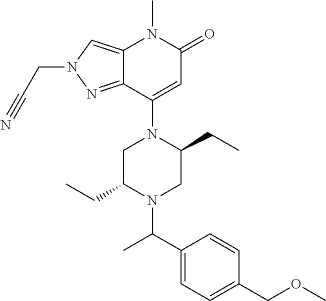 [SMILES: CC[C@H]1CN(C(C)c2ccc(COC)cc2)[C@H](CC)CN1c1cc(=O)n(C)c2cn(CC#N)nc12]

### Step A: methyl 4-(1-((tert-butyldimethylsilyl)oxy)ethyl)benzoate

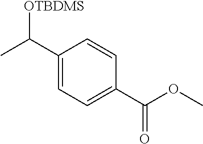 [SMILES: COC(=O)c1ccc(C(C)O[Si](C)(C)C(C)(C)C)cc1]

To a stirred solution of methyl 4-(1-hydroxyethyl)benzoate (5 g, 27.78 mmol) and imidazole (2.27 g, 33.33 mmol) in DCM (200 mL) was added TBDMSCl (4.17 g, 27.78 mmol). After then, the mixture was stirred for 3 h. The mixture was diluted with CH₂Cl₂ (20 mL), then washed with H₂O, brine, dried over Na₂SO₄, concentrated and purified by flash column chromatography to give the titled compound (8 g, 98%) as colorless oil. MS: M/e 295 (M+1)⁺.

### Step B: (4-(1-((tert-butyldimethylsilyl)oxy)ethyl)phenyl)methanol

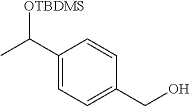 [SMILES: CC(O[Si](C)(C)C(C)(C)C)c1ccc(CO)cc1]

To a stirred solution of methyl 4-(1-((tert-butyldimethylsilyl)oxy)ethyl)benzoate (8 g, 27.21 mmol) in THF (200 mL) was added aluminum(III) lithium hydride (1.0 M, 32.65 mmol, 32.65 mL) at 0° C. After then, the mixture was stirred at r.t for 5 hours. The mixture was quenched with a.q NaOH, filtered and extracted with CH₂Cl₂. The combined organic layers were washed with brine, dried over Na₂SO₄, concentrated to give the titled compound (6.7 g, 92%) as a white solid. MS: M/e 267 (M+1)⁺.

### Step C: tert-butyl(1-(4-(methoxymethyl)phenyl)ethoxy)dimethylsilane

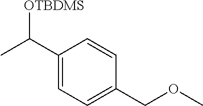 [SMILES: COCc1ccc(C(C)O[Si](C)(C)C(C)(C)C)cc1]

To a mixture of (4-(1-((tert-butyldimethylsilyl)oxy)ethyl)phenyl)methanol (200 mg, 0.75 mmol) in THF (20 mL) was added NaH (36 mg, 0.90 mmol) at 0° C. and stirred for 1 h, Then iodomethane (159 mg, 1.13 mmol) was added and stirred at r.t for 2 h. The reaction mixture was extracted with EA and washed with brine, dried over Na₂SO₄, concentrated and purified by flash column chromatography to give the titled compound (150 mg, 71%) as colorless oil. MS: M/e 281 (M+1)⁺.

### Step D: 1-(4-(methoxymethyl)phenyl)ethan-1-ol

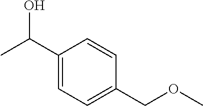

To a solution of tert-butyl(1-(4-(methoxymethyl)phenyl)ethoxy)dimethylsilane (150 mg, 0.54 mmol) in THF (5 mL) was added TBAF (1 mL, 1 mmol). The reaction mixture was stirred at room for 3 hours. The reaction mixture was diluted with water and extracted with EA (80 mL×2). The combined organic layers were washed with brine, dried over Na₂SO₄, filtered and concentrated to dryness. The resulting residue was purified by flash column chromatography to give the titled compound (80 mg, 90%).

### Step E: 2-(7-((2S,5R)-2,5-diethyl-4-(1-(4-(methoxymethyl)phenyl)ethyl)piperazin-1-yl)-4-methyl-5-oxo-4,5-dihydro-2H-pyrazolo[4,3-b]pyridin-2-yl)acetonitrile

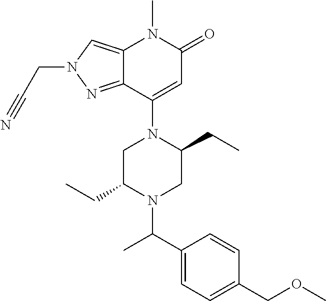 [SMILES: CC[C@H]1CN(C(C)c2ccc(COC)cc2)[C@H](CC)CN1c1cc(=O)n(C)c2cn(CC#N)nc12]

To a solution of 2-(7-((2S,5R)-2,5-diethylpiperazin-1-yl)-4-methyl-5-oxo-4,5-dihydro-2H-pyrazolo[4,3-b]pyridin-2-yl)acetonitrile (50 mg, 0.15 mmol) in CH₃CN (3 mL) and was added 1-(4-(methoxymethyl)phenyl)ethan-1-ol (30 mg, 0.18 mmol), (cyanomethyl)trimethylphosphonium iodide (112 mg, 0.46 mmol) and DIPEA (98 mg, 0.76 mmol). The resulting mixture was stirred at 105° C. overnight. The reaction solvent was removed under reduce pressure to afford crude product. The resulting residue was purified Prep-HPLC (Method A) to give the titled compound (22 mg, 30%).1H NMR (400 MHz, CD₃OD) δ 7.92 (d, J=2.8 Hz, 1H), 7.38 (dd, J=12.6, 8.1 Hz, 2H), 7.31 (dd, J=13.6, 8.1 Hz, 2H), 5.54 (d, J=2,4 Hz, 1H), 5.46 (d, J=4.6 Hz, 2H), 4.45 (d, J=5.9 Hz, 2H), 3.81-3.59 (m, 1H), 3.53-3.43 (m, 1H), 3.43 (s, 3H), 3.37 (d, J=10.3 Hz, 3H), 3.30-3.21 (m, 2H), 3.17-3.00 (m, J=66.9, 10.0 Hz, 1H), 2.91-2.61 (m, . =120.9, 12.9 Hz, 1H), 2,52-2.33 (m, 1H), 2.17-1.87 (m, 1H), 1.85-1.50 (m, 3H), 1.33 (dd, J=19.5, 6.5 Hz, 3H), 0.99 (dt, J=29.7, 7.4 Hz, 3H), 0.65 (dt, J=30.6, 7.3 Hz, 3H) ppm. MS: M/e 477 (M+1)⁺.

### Compound A206: 2-(but-2-yn-1-yl)-7-((2S,5R)-2,5-diethyl-4-(1-(quinoxalin-2-yl)ethyl)piperazin-1-yl)-4-methyl-2,4-dihydro-5H-pyrazolo[4,3-b]pyridin-5-one

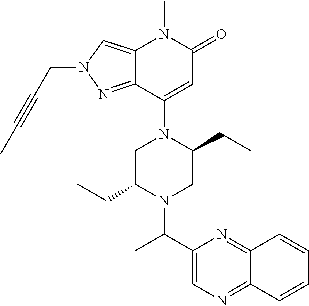 [SMILES: CC#CCn1cc2c(n1)c(N1C[C@@H](CC)N(C(C)c3cnc4ccccc4n3)C[C@@H]1CC)cc(=O)n2C]

### Step A: 1-(quinoxalin-2-yl)ethan-1-ol

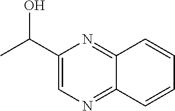

A mixture of quinoxaline-2-carbaldehyde (1 g, 6.3 mmol) in THF (20 mL) at 0° C. was added methyl magnesium bromide (3.1 mL, 3M, 9.4 mol). The reaction mixture was stirred at room temperature for 20 mins. The reaction was quenched by adding aqueous NH₄Cl. The resulting mixture was extracted with EA, and then concentrated by using a rotary evaporator, to give the titled compound (1 g). MS: M/e 175 (M+1)⁺.

### Step B: t tert-butyl (2S,5R)-2,5-diethyl-4-(1-(quinoxalin-2-yl)ethyl)piperazine-1-carboxylate

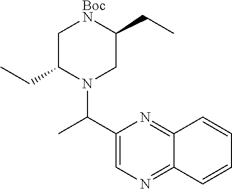 [SMILES: CC[C@H]1CN(C(C)c2cnc3ccccc3n2)[C@H](CC)CN1C(=O)OC(C)(C)C]

A sealed tube was charged with 1-(quinoxalin-2-yl)ethan-1-ol (0.8 g, 4.6 mmol), tert-butyl (2S,5R)-2,5-diethylpiperazine-1-carboxylate (1.6 g, 6.8 mmol), (cyanomethyl)trimethylphosphonium iodide(3.3 g, 13.8 mmol), DIPEA(5.9 g, 46 mmol ) and acetonitrile(15 ml ). The reaction mixture was stirred at 105° C. overnight and cooled to room temperature. The solvent was removed. Water was added and the aqueous was extracted with EA. The combined organic layers were washed with brine, dried over Na₂SO₄, and concentrated to dryness. The resulting residue was purified by flash column chromatography (EA/PE) to give the titled compound (0.4 g, 22%). MS: M/e 399 (M+1)⁺.

### Step C: 2-(1-((2R,5S)-2,5-diethylpiperazin-1-yl)ethyl)quinoxaline

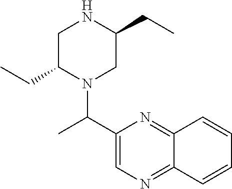 [SMILES: CC[C@H]1CN(C(C)c2cnc3ccccc3n2)[C@H](CC)CN1]

To a solution of tert-butyl (2S,5R)-2,5-diethyl-4-(1-(quinoxalin-2-yl)ethyl)piperazine-1-carboxylate(0.7 g, 1.8 mmol) in DCM (10 mL) was added TFA (3 ml). The reaction mixture was stirred at RT for 4 hours, concentrated to dryness. The resulting residue was dissolved into water. The resulting mixture was extracted with EA. The aqueous layer was adjusted pH to 9-10 with saturated Na₂CO₃ aq. and extracted with DCM (50 mL×2). The combined organic layers were washed with brine, dried over Na₂SO₄ and concentrated to dryness. The resulting residue (0.32 g) was used in the next step without further purification. MS: M/e 299 (M+1)⁺.

### Step D: 7-((2S,5R-2,5-diethyl-4-(1-(quinoxalin-2-yl)ethyl)piperazin-1-yl)-4-methyl-2-(tetrahydro-2H-pyran-2-yl)-2,4-dihydro-5H-pyrazolo[4,3-b]pyridin-5-one

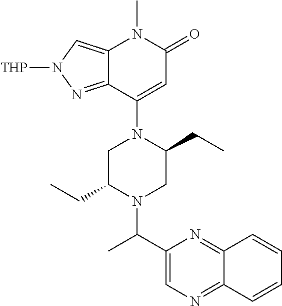

To a solution of 2-(1-((2R,5S)-2,5-diethylpiperazin-1-yl)ethyl)quinoxaline (320 mg, 1.07 mmol) in DMA (8 mL) was added 4-methyl-5-oxo-2-(tetrahydro-2H-pyran-2-yl)-4,5-dihydro-2H-pyrazolo[4,3-b]pyridin-7-yl trifluoromethanesulfonate (0.49 g, 1,28 mmol) and DIPEA(414 mg, 3.2 mmol). The reaction mixture was stirred at 100° C. overnight. The reaction mixture was quenched with water. The resulting mixture was extracted with DCM (50 mL×2). The combined organic layers were washed with brine, dried over Na₂SO₄, filtered and concentrated to dryness. The resulting residue was purified by flash column chromatography (MeOH/DCM) to give the titled compound (0.27 g, 47%). MS: M/e 530 (M+1)⁺.

### Step E: 7-((2S,5R 2,5-diethyl-4-(1-(quinoxalin-2-yl)ethyl)piperazin-1-yl)-4-methyl-2,4-dihydro-5H-pyrazolo[4,3-b]pyridin-5-one

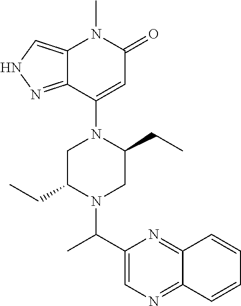 [SMILES: CC[C@H]1CN(C(C)c2cnc3ccccc3n2)[C@H](CC)CN1c1cc(=O)n(C)c2c[nH]nc12]

To a solution of 7-((2S,5R)-2,5-diethyl-4-(1-(quinoxalin-2-yl)ethyl)piperazin-1-yl)-4-methyl-2-(tetrahydro-2H-pyran-2-yl)-2,4-dihydro-5H-pyrazolo[4,3-b]pyridin-5-one (270 mg, 1.9 mmol) in DCM (8 mL) was added TFA (3 ml). The reaction mixture was stirred at RT for 3 hours, concentrated to dryness. The resulting residue was dissolved into water. The resulting mixture was extracted with EA. The aqueous layer was adjusted pH to 9-10 with saturated Na₂CO₃ aq. and extracted with DCM (50 mL×2). The combined organic layers were washed with brine, dried over Na₂SO₄, and concentrated to dryness. The resulting residue (0.16 g) was used in the next step without further purification. MS: M/e 446 (M+1)”.

### Step F: 2-(7-((2R,5R)-2,5-dimethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile

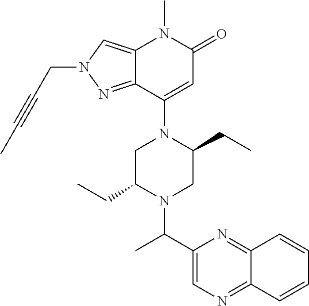 [SMILES: CC#CCn1cc2c(n1)c(N1C[C@@H](CC)N(C(C)c3cnc4ccccc4n3)C[C@@H]1CC)cc(=O)n2C]

To a solution of 7-((2S,5R)-2,5-diethyl-4-(1-(quinoxalin-2-yl)ethyl)piperazin-1-yl)-4-methyl-2,4-dihydro-5H-pyrazolo[4,3-b]pyridin-5-one (80 mg, 0.18 mmol) and K₂CO₃ (75 mg, 0.54 mmol) in DMF (3 mL) was added 1-bromobut-2-yne (119 mg, 0.89 mmol). The reaction mixture was stirred at room temperature for 2 days. The reaction mixture was diluted with DCM. The combined organic layers were washed with water, dried over Na₂SO₄, filtered and concentrated to dryness. The resulting residue was purified by flash column chromatography (DCM/MeOH) to give the titled compound(crude), which was further separated into Compound A206a (7 mg) and Compound A206b (11 mg) by Prep-HPLC (Method A).

Compound A206a (the earlier peak): ¹H NMR (400 MHz, CD₃OD) δ 9.16 (s, 1H), 8.08 (t, J=9.5 Hz, 2H), 7.90 (s, 1H), 7.89-7.77 (m, 2H), 5.52 (s, 1H), 5.04 (d, J=2.3 Hz, 2H), 4.77-4.10 (m, 2H), 4.04 (q, J=6.9 Hz, 1H), 3.58 (d, J=12.6 Hz, 1H), 3.44 (s, 3H), 3.23 (d, J=10.5 Hz, 1H), 2.86 (dd, J=12.2, 3.5 Hz, 1H), 2.25 (d, J=12.0 Hz, 1H), 1.98-1.87 (m, 1H), 1.85 (d, J=2.3 Hz, 3H), 1.80-1.56 (m, 3H), 1.52 (d, J=6.8 Hz, 3H), 1.05 (t, J=7.3 Hz, 3H), 0.57 (t, J=7.4 Hz, 3H) ppm. MS: M/e 498 (M+1)⁺.

Compound A206b (the later peak): ¹H NMR (400 MHz, CD₃OD) δ 9.10 (s, 1H), 8.08 (q, J=8.0 Hz, 2H), 7.89 (s, 1H), 7.84 (p, J=8.1 Hz, 2H), 5.54 (s, 1H), 5.02 (d, J=2.1 Hz, 2H), 4.77-4.31 (m, 2H), 4.25 (q, J=6.6 Hz, 1H), 3.44 (s, 3H), 3.37 (d, J=12.9 Hz, 1H), 3.06-2.96 (m, 2H), 2,47 (s, 1H), 2.01 (dq, J=15.8, 7.7 Hz, 1H), 1.90-1.75 (m, 4H), 1.65 (dd, J=14.4, 7.1 Hz, 2H), 1.55 (d, J=6.7 Hz, 3H), 0.86 (t, J=7.4 Hz, 3H), 0.75 (t, J=7.4 Hz, 3H) ppm. MS: M/e 498 (M+1)⁺.

### Assay

### Biochemical DGK IC₅₀ Assays

Enzymatic reactions of DGKζ, DGKα and DGKδ were performed using ADP-Glo assay with lipid micelle substrate. Full length DGK (in-house protein M1-V929 with SEQ ID No: 2) was expressed in baculovirus expression system. Full length DGKα (D21-10BG, SignalChem) and full length DGKδ (D23-10G, SignalChem) were purchased. Lipid micelle was prepared by dissolving DAG (Sigma, 317505-10MG) and PS (Sigma, P7769-100MG) with chloroform which was furtherly removed by rotary evaporation. The resulted product was resuspended in buffer containing 25 mM HEPES pH 7.0, 0.5 mM EDTA and 160 mM Octyl P-D glucopyranoside by vigorous mixing and ultrasonic (IID, Scientz).

Final concentration in reaction

| Enzyme | Enzyme (nM) | ATP (μM) | DAG (μM) |
|---|---|---|---|
| DGKα | 7.5 | 140 | 80 |
| DGKδ | 10 | 140 | 80 |
| DGKζ | 0.5 | 140 | 80 |

The inhibition activities testing for the compound disclosed herein were carried out at room temperature in assay buffer containing 50 mM HEPES, 10 mM MgCl₂, 0.01% BSA, 0.1 mM Na₃VO₄, 0.005% Tween-20 and 0.01 mM CaCl₂. Compounds in DMSO were dispensed into wells of a black 384 well plate (Corning 4514) using D300e digital dispenser (Tecan). The ranges of compounds final concentration were 1.55-10000 nM or 23.3-150000 nM. 3 μL 2× enzyme solution was added to wells. After incubation for 1 hour, 3 μL 2× substates solution containing 160 μM DAG and 280 μM ATP was added to the wells to initiate reaction. After 1 hour reaction, 5 μL ADP-Glo reagent (Promga V9101) was added and incubated for 40 minutes. 10 μL Kinase Detection reagent was added and incubated for 30 minutes. Luminescence was measured on a microplate reader (PHERAstar FSX, BMG labtech). The IC₅₀s are calculated based on inhibition of enzyme activity in the presence of increasing concentrations of compounds. Selected compounds had no inhibitory activity on DGKδ. IC₅₀s of the compounds disclosed herein for DGKζ and DGKα are shown in Table 1.

### Baculovirus Expression of Human DGKζ

Human His-TEV-DGK-zeta-pFastBac1 and human baculovirus samples was generated using the Bac-to-Bac baculovirus expression system (Invitrogen) according to the manufacturer's protocol. The DNA used for expression of DGK-zeta, have SEQ ID Nos: 1. Baculovirus amplification was achieved using infected SF9 cells at 1:2000 virus/cells ratios, and grown for 96 hours at 27° C. post-transfected.

The expression scaled up for each protein was carried out in the flask 3 L from CORNING. 4 L of 3×10⁶ cells/mL Sf9 cells (Expression System, Invitrogen) grown in SF900™ II SFM insect medium (Expression System) was infected with virus stock at 1:200 virus/cells ratio, and grown for 48 hours at 27° C. post-transfection. The infected cell culture was harvested by centrifugation at 6000 rpm for 15 min 4° C. in a SORVALL LYNX6000 centrifuge. The cell pellets were stored at −80° C.

### Purification of Human DGK-Zeta

Full length human DGKζ produced as described above, was purified from Sf9 baculovirus-infected insect cell paste. The cells were lysed using sonication method, and the lysates were clarified by centrifugation. The clarified lysates were purified to -90% homogeneity, using two successive column chromatography steps on an AKTA Purifier system. The two steps column chromatography included nickel affinity resin capture (i.e. Ni-NTA Agarose, Qiagen), followed by size exclusion chromatography (i.e. Hiload16/60 Superdex200 prep grade, GE Healthcare. The protein was delivered and stored at −80° C. The formulation buffers were identical for the protein: 25 mM Tris, 150 mmol/L NaCl, 2 mM DTT, pH8.0.

SEQ ID No: 1 is Nucleotide sequence encoding His-TEV-hDGKζ-(M1-V929):
ATGCATCACCATCACCATCACCATCACGAGAACCTGTACTTCCAGGGATCC
ATGGAACCCCGTGATGGTAGCCCCGAAGCTCGTAGCTCCGATTCCGAGTCCGCCAGCGC
TTCCTCCTCCGGTAGCGAACGTGACGCTGGTCCCGAGCCCGACAAAGCTCCCCGTCGTC
TGAATAAGCGCCGTTTTCCCGGTCTCCGTCTGTTCGGCCACCGCAAGGCCATCACTAAG
TCCGGTCTCCAGCATCTGGCTCCTCCTCCTCCTACCCCCGGTGCTCCTTGCTCCGAATCC
GAGCGCCAGATTCGCTCCACTGTGGATTGGTCCGAAAGCGCCACCTATGGTGAGCATAT
CTGGTTCGAGACCAACGTCTCCGGCGACTTCTGTTATGTCGGTGAGCAATACTGTGTGG
CTCGTATGCTGCAGAAGTCCGTGTCCCGCCGTAAATGCGCCGCTTGCAAAATCGTGGTC
CATACCCCTTGCATCGAGCAACTGGAGAAAATCAACTTCCGCTGCAAGCCCAGCTTTCG
TGAGTCCGGTTCCCGCAACGTGCGCGAACCTACTTTCGTGCGCCACCACTGGGTGCATC
GTCGTCGCCAAGACGGCAAATGCCGCCACTGCGGCAAAGGTTTTCAGCAGAAATTCAC
CTTCCACAGCAAGGAGATCGTCGCCATCAGCTGCAGCTGGTGCAAACAAGCTTACCATT
CCAAAGTGAGCTGCTTCATGCTCCAGCAGATCGAAGAGCCTTGCTCTCTGGGTGTGCAT
GCTGCTGTCGTGATTCCCCCTACTTGGATTCTGCGTGCTCGCCGTCCCCAGAACACTCTG
AAGGCCTCCAAAAAGAAGAAGCGCGCCAGCTTCAAGCGTAAGAGCTCCAAAAAGGGT
CCCGAAGAGGGCCGTTGGCGTCCCTTCATCATCCGCCCTACTCCTTCCCCTCTGATGAAG
CCTCTGCTGGTCTTCGTCAACCCTAAGAGCGGCGGCAACCAAGGTGCTAAAATCATCCA
GTCCTTCCTCTGGTATCTGAACCCTCGTCAAGTGTTCGACCTCAGCCAAGGCGGTCCTA
AGGAGGCTCTGGAGATGTACCGCAAGGTCCACAATCTGCGCATCCTCGCTTGTGGTGGC
GATGGCACCGTGGGCTGGATTCTGTCCACTCTGGACCAACTGCGTCTGAAACCTCCCCC
CCCCGTGGCTATTCTGCCTCTCGGTACCGGCAACGATCTGGCTCGTACTCTGAATTGGGG
TGGTGGCTACACCGATGAGCCCGTGTCCAAGATTCTGTCCCACGTCGAAGAAGGCAATG
TCGTCCAACTGGACCGTTGGGACCTCCACGCCGAACCCAACCCCGAGGCTGGCCCCGA
GGACCGTGACGAGGGCGCTACTGACCGTCTGCCCCTCGACGTCTTCAATAATTACTTCTC
TCTGGGCTTTGACGCTCACGTGACTCTGGAATTTCATGAAAGCCGCGAGGCCAACCCCG
AGAAGTTCAATTCCCGTTTCCGCAACAAGATGTTCTACGCTGGCACCGCCTTCAGCGAC
TTCCTCATGGGCTCCAGCAAGGACCTCGCTAAGCATATCCGCGTGGTGTGCGATGGCATG
GATCTGACCCCTAAGATCCAAGATCTGAAGCCCCAATGTGTCGTGTTTCTGAACATCCCC
CGCTACTGCGCTGGTACTATGCCTTGGGGCCATCCCGGTGAACACCATGACTTCGAACCT
CAGCGTCATGATGACGGCTATCTGGAGGTGATCGGTTTCACCATGACCTCCCTCGCTGCT
CTGCAAGTGGGTGGCCACGGCGAACGTCTGACTCAATGCCGCGAGGTGGTGCTGACCA
CCAGCAAAGCCATCCCCGTCCAAGTGGATGGTGAGCCTTGCAAGCTGGCCGCCTCCCGT
ATCCGTATCGCTCTCCGCAATCAAGCTACCATGGTCCAGAAGGCCAAACGCCGCAGCGC
TGCTCCTCTCCACAGCGACCAACAACCCGTCCCCGAACAGCTGCGCATCCAAGTGTCCC
GTGTCAGCATGCATGACTACGAGGCTCTGCACTACGACAAGGAACAGCTGAAGGAAGC
CAGCGTGCCTCTGGGTACTGTGGTCGTGCCCGGTGACAGCGATCTGGAGCTCTGCCGTG
CCCACATCGAGCGTCTGCAGCAAGAGCCCGACGGTGCTGGTGCCAAGAGCCCTACTTG
CCAAAAACTCTCCCCCAAGTGGTGTTTCCTCGACGCTACCACCGCCAGCCGCTTCTACC
GCATTGATCGCGCCCAAGAGCATCTGAACTATGTCACCGAGATCGCTCAAGACGAGATC
TACATCCTCGACCCCGAACTCCTCGGTGCTAGCGCCCGTCCCGACCTCCCCACTCCTACC
TCCCCTCTGCCCACTTCCCCTTGTTCCCCCACCCCTCGTAGCCTCCAAGGTGATGCTGCC
CCTCCTCAAGGTGAGGAGCTCATTGAGGCCGCTAAGCGTAACGATTTCTGCAAGCTCCA
AGAGCTGCATCGTGCTGGTGGCGACCTCATGCACCGCGATGAGCAGAGCCGCACTCTGC
TGCACCACGCTGTGTCCACTGGTAGCAAGGACGTGGTGCGCTATCTGCTGGACCACGCT
CCTCCCGAGATCCTCGACGCTGTGGAAGAAAACGGCGAGACTTGCCTCCACCAAGCTG
CTGCTCTGGGTCAACGTACCATCTGCCACTACATCGTCGAAGCTGGTGCTTCTCTGATGA
AGACCGACCAGCAAGGTGATACTCCCCGTCAGCGCGCCGAGAAAGCCCAAGACACCGA
ACTGGCTGCCTATCTGGAGAACCGTCAGCACTACCAGATGATTCAGCGTGAAGACCAAG
AGACCGCCGTGTAA
SEQ ID No: 2 is Amino acid sequence of His-TEV-hDGKζ-(M1-V929):
MEPRDGSPEARSSDSESASASSSGSERDAGPEPDKAPRRLNKRRFPGLRLFGH
RKAITKSGLQHLAPPPPTPGAPCSESERQIRSTVDWSESATYGEHIWFETNVSGDFCYVGEQ
YCVARMLQKSVSRRKCAACKIVVHTPCIEQLEKINFRCKPSFRESGSRNVREPTFVRHHWV
HRRRQDGKCRHCGKGFQQKFTFHSKEIVAISCSWCKQAYHSKVSCFMLQQIEEPCSLGVHA
AVVIPPTWILRARRPQNTLKASKKKKRASFKRKSSKKGPEEGRWRPFIIRPTPSPLMKPLLVF
VNPKSGGNQGAKIIQSFLWYLNPRQVFDLSQGGPKEALEMYRKVHNLRILACGGDGTVGW
ILSTLDQLRLKPPPPVAILPLGTGNDLARTLNWGGGYTDEPVSKILSHVEEGNVVQLDRWDL
HAEPNPEAGPEDRDEGATDRLPLDVFNNYFSLGFDAHVTLEFHESREANPEKFNSRFRNKM
FYAGTAFSDFLMGSSKDLAKHIRVVCDGMDLTPKIQDLKPQCVVFLNIPRYCAGTMPWGHP
GEHHDFEPQRHDDGYLEVIGFTMTSLAALQVGGHGERLTQCREVVLTTSKAIPVQVDGEPC
KLAASRIRIALRNQATMVQKAKRRSAAPLHSDQQPVPEQLRIQVSRVSMHDYEALHYDKE
QLKEASVPLGTVVVPGDSDLELCRAHIERLQQEPDGAGAKSPTCQKLSPKWCFLDATTASR
FYRIDRAQEHLNYVTEIAQDEIYILDPELLGASARPDLPTPTSPLPTSPCSPTPRSLQGDAAPP
QGEELIEAAKRNDFCKLQELHRAGGDLMHRDEQSRTLLHHAVSTGSKDVVRYLLDHAPPEI
LDAVEENGETCLHQAAALGQRTICHYIVEAGASLMKTDQQGDTPRQRAEKAQDTELAAYL
ENRQHYQMIQREDQETAV*

TABLE 1

| | DGKζ ADP-Glo | DGKα ADP-Glo |
|---|---|---|
| Compound | assay IC₅₀(nM) | assay IC₅₀(nM) |
| A1a | 1440 | 23 |
| A1b | >10000 | 734 |
| A2a | 1170 | 73 |
| A2b | 3860 | 896 |
| A3a | 227 | 13633 |
| A3b | 551 | 5900 |
| A4a | 569 | 2690 |
| A4b | 3170 | 4840 |
| A5 | 73 | 497 |
| A6 | 219 | 8530 |
| A7 | 152 | 129 |
| A8 | 229 | 1000 |
| A9 | 339 | 2110 |
| A10 | 104 | 162 |
| A11 | 500 | 79 |
| A12 | 92 | 440 |
| A13 | 323 | 838 |
| A14 | 63 | 8410 |
| A15 | 219 | 193 |
| A16 | 170 | 204 |
| A17 | 315 | 343 |
| A18 | 97 | 249 |
| A19a | 130 | 22 |
| A19b | 4900 | 289 |
| A20 | 312 | 378 |
| A21a | 1470 | 2890 |
| A21b | >10000 | >150000 |
| A22 | 465 | 833 |
| A23a | 3740 | 2350 |
| A23b | 534 | 138 |
| A24 | 427 | 100 |
| A25a | 246 | 3380 |
| A25b | 2200 | 6020 |
| A26a | 4090 | 1440 |
| A26b | >10000 | 24700 |
| A27 | 202 | 50 |
| A28 | 4830 | 10000 |
| A29 | 3930 | 2600 |
| A30 | 3690 | 666 |
| A31 | 213 | 615 |
| A32 | 244 | 183 |
| A33a | 45 | 49 |
| A33b | 770 | 344 |
| A34a | 75 | 37 |
| A34b | 2940 | 596 |
| A35a | 234 | 131 |
| A35b | 2420 | >150000 |
| A36a | 57 | 164 |
| A36b | 946 | 2110 |
| A37 | 202 | 541 |
| A38a | 126 | 810 |
| A38b | 1180 | 2347 |
| A39a | 464 | 33 |
| A39b | 2360 | 237 |
| A40 | 110 | 4080 |
| A41 | 151 | 347 |
| A42a | 315 | 1960 |
| A42b | 5060 | 6640 |
| A43a | 21 | 260 |
| A43b | 362 | 1020 |
| A44a | 110 | 225 |
| A44b | 1510 | 841 |
| A45a | 30 | 202 |
| A45b | 865 | 728 |
| A46a | 6.4 | 406 |
| A46b | 396 | 913 |
| A47a | 88 | 1190 |
| A47b | 1120 | 3520 |
| A48a | 59 | 240 |
| A48b | 108 | 788 |
| A49a | 64 | 10600 |
| A49b | 1800 | 8160 |
| A50 | 83 | 703 |
| A5la | 138 | 26 |
| A51b | 1800 | 337 |
| A52 | 99 | 118 |
| A53 | 331 | 4490 |
| A54a | 33 | 76 |
| A54b | 1890 | 1910 |
| A55 | 653 | 2343 |
| A56 | 271 | 2583 |
| A57 | 51 | 14333 |
| A58 | 94 | 487 |
| A59 | 536 | 137 |
| A60 | 643 | 38000 |
| A61 | 298 | 143 |
| A62 | 154 | 21 |
| A63 | 21 | 167 |
| A64 | 7.8 | 1040 |
| A65 | 66 | 295 |
| A66 | 96 | 2250 |
| A67 | 71 | 245 |
| A68a | 49 | 97 |
| A68b | 2550 | 1390 |
| A69 | 20 | 4733 |
| A70 | 31 | 12367 |
| A71 | 4.0 | 1830 |
| A72 | 33 | 27 |
| A73 | 126 | 91 |
| A74 | 475 | 5820 |
| A75a | 139 | 74 |
| A75b | 5050 | 1110 |
| A76 | 179 | 1600 |
| A77a | 54 | 178 |
| A77b | 859 | 744 |
| A78 | 1870 | 25000 |
| A79a | 589 | 3690 |
| A79b | 8320 | 9770 |
| A80a | 939 | 140 |
| A80b | 6900 | 3040 |
| A81a | 200 | 136 |
| A81b | 7130 | 2990 |
| A82a | 486 | 109 |
| A82b | 8520 | 3050 |
| A83a | 8790 | 648 |
| A83b | 1800 | 90 |
| A84a | 43 | 68 |
| A84b | 3180 | 1170 |
| A85a | 78 | 170 |
| A85b | 3500 | 2040 |
| A86a | 20 | 4710 |
| A86b | 2310 | 12500 |
| A87a | 2820 | 2010 |
| A87b | >10000 | 35200 |
| A88 | 10 | 51 |
| A89 | 34 | 28 |
| A90 | 20 | 75 |
| A90a | 11 | 30 |
| A90b | 204 | 675 |
| A91a | 14 | 263 |
| A91b | 1040 | 3340 |
| A92a | 9.1 | 108 |
| A92b | 265 | 1020 |
| A93 | 99 | 493 |
| A94 | 974 | 1850 |
| A95 | 64 | 52 |
| A96a | 46 | 180 |
| A96b | 1920 | 9180 |
| A97 | 256 | 502 |
| A98a | 108 | 5510 |
| A98b | 2870 | 9470 |
| A99a | 3520 | 1740 |
| A99b | 8.6 | 24 |
| A100a | 46 | 85 |
| A100b | 2610 | 2890 |
| A101a | 19 | 2610 |
| A101b | 1240 | 17700 |
| A102a | 1.6 | 41 |
| A102b | 84 | 1240 |
| A103a | 14 | 92 |
| A103b | 629 | 1350 |
| A104a | 6.5 | 3150 |
| A104b | 220 | 18400 |
| A105 | 157 | 58 |
| A106a | 9 | 65 |
| A106b | 600 | 932 |
| A107a | 26 | 108 |
| A107b | 2050 | 872 |
| A108 | 11 | 2650 |
| A109 | 18 | 2430 |
| A110a | 31 | 469 |
| A110b | 1720 | 10500 |
| A111a | >10000 | 2540 |
| A111b | 55 | 1253 |
| A112a | 78 | 65 |
| A112b | >10000 | 497 |
| A113a | 48 | 11 |
| A113b | 7570 | 696 |
| A114a | 83 | 32 |
| A114b | 3200 | 391 |
| A115a | 431 | 269 |
| A115b | 6490 | 1250 |
| A116a | 181 | 2330 |
| A116b | >10000 | 21000 |
| A117a | 33 | 5 |
| A117b | 1130 | 65 |
| A118 | 232 | 20 |
| A119 | 859 | 193 |
| A120a | 222 | 55 |
| A120b | 4110 | 225 |
| A121 | 1470 | 3080 |
| A122 | >10000 | 17100 |
| A123 | 11 | 111 |
| A124 | 39 | 433 |
| A125a | 34 | 1240 |
| A125b | 1190 | 25200 |
| A126a | 1320 | 1370 |
| A126b | 51 | 93 |
| A127a | 71 | 357 |
| A127b | 3280 | 3360 |
| A128a | 60 | 375 |
| A128b | 694 | 4480 |
| A129a | 626 | 2100 |
| A129b | 45 | 192 |
| A130 | 46 | 215 |
| A131 | 26 | 246 |
| A132 | 139 | 63 |
| A133a | 1520 | 3280 |
| A133b | 24 | 61 |
| A134a | 3920 | 3390 |
| A134b | 22 | 108 |
| A135a | 96 | 86 |
| A135b | 3750 | 483 |
| A136a | >10000 | 28500 |
| A136b | 123 | 283 |
| A137 | 118 | 13 |
| A138a | 143 | 311 |
| A138b | 3280 | 2040 |
| A139a | 116 | 64 |
| A139b | 3930 | 1080 |
| A140a | 3380 | 840 |
| A140b | 45 | 93 |
| A141a | 17.6 | 1390 |
| A141b | 641 | 6190 |
| A142a | 1350 | 1580 |
| A142b | 20 | 108 |
| A143a | 2150 | 3180 |
| A143b | 39 | 153 |
| A144a | 1300 | 270 |
| A144b | 135 | 18 |
| A145a | 47 | 545 |
| A145b | 939 | 3350 |
| A146a | 1750 | 2400 |
| A146b | 104 | 205 |
| A147a | 152 | 180 |
| A147b | 1510 | 1570 |
| A148a | 2310 | 1280 |
| A148b | 45 | 36 |
| A149a | 2720 | 1110 |
| A149b | 93 | 43 |
| A150a | >10000 | 7140 |
| A150b | 267 | 124 |
| A151a | 4610 | 7320 |
| A151b | 7.5 | 156 |
| A152a | 3090 | 1910 |
| A152b | 65.5 | 45 |
| A153a | >10000 | 2720 |
| A153b | 195 | 72 |
| A154a | >10000 | 505 |
| A154b | 55 | 12 |
| A155 | 74 | 378 |
| A155a | 20 | 1140 |
| A155b | 1970 | 139 |
| A156a | 5130 | >10000 |
| A156b | 23 | 310 |
| A157a | >10000 | >10000 |
| A157b | 166 | 183 |
| A158a | >10000 | 330 |
| A158b | 91 | 21 |
| A159a | 56 | 38 |
| A159b | 1120 | 40. |
| A160a | 4120 | 5970 |
| A160b | 15 | 47 |
| A161a | >10000 | >10000 |
| A161b | 18 | 32 |
| A162a | 51 | 104 |
| A162b | 3270 | 2430 |
| A163a | >10000 | 1900 |
| A163b | 426 | 71 |
| A164a | >10000 | 7300 |
| A164b | 88 | 134 |
| A165a | 12 | 96 |
| A165b | 1580 | 2440 |
| A166a | 227 | 93 |
| A166b | >10000 | 1630 |
| A167a | >10000 | >10000 |
| A167b | 23 | 219 |
| A168a | 3600 | 3410 |
| A168b | 69 | 54 |
| A169a | 211 | 3210 |
| A169b | 9279 | >10000 |
| A170a | >10000 | >10000 |
| A170b | 52.5 | 109 |
| A171a | 116 | 107 |
| A171b | 5430 | 2687 |
| A172a | 108 | 158 |
| A172b | >10000 | 7828 |
| A173a | 19 | 168 |
| A173b | 2952 | >10000 |
| A174a | 8853 | 6473 |
| A174b | 8.9 | 53 |
| A175 | 79 | 32 |
| A176 | 53 | 82 |
| A177a | 134 | 96 |
| A177b | 2850 | 568 |
| A178a | 43 | 157 |
| A178b | 150 | 3433 |
| A179a | 1410 | 15900 |
| A179b | 323 | 3190 |
| A180 | 1180 | 2450 |
| A181 | 178 | 470 |
| A182 | 281 | 54 |
| A183 | 517 | 48.2 |
| A184a | >10000 | 354 |
| A184b | >10000 | 15700 |
| A185 | 66.4 | 164 |
| A186a | 841 | 53 |
| A186b | >10000 | 1220 |
| A187a | >10000 | 1990 |
| A187b | >10000 | 11900 |
| A188a | 219 | 2140 |
| A188b | 2010 | 9080 |
| A189a | 2190 | 1080 |
| A189b | 8540 | 11800 |
| A190 | 651 | 45 |
| A191 | 285 | <23.3 |
| A192 | 579 | 729 |
| A193a | 2870 | 515 |
| A193b | >10000 | 6510 |
| A194a | 1450 | 291 |
| A194b | 6050 | 2530 |
| A195a | 1410 | 774 |
| A195b | >10000 | 4210 |
| A196 | 552 | 1020 |
| A197 | 252 | 711 |
| A198 | 5560 | 91 |
| A199 | 112 | 2170 |
| A200 | 991 | <23.3 |
| A201 | 123 | 43 |
| A202 | 3210 | 352 |
| A203a | 2690 | 1110 |
| A203b | 7610 | 11600 |
| A204a | 31 | 467 |
| A204b | 971 | 7300 |
| A205 | 142 | 215 |
| A206a | 8300 | 480 |
| A206b | 103 | 25 |

### Cell Culture and DGK4α/ζ Knockout Jurkat Cell Line Construction

Jurkat cells and human PBMC were maintained in RPMI 1640 medium (Gibco) supplemented with 10%/ fetal bovine serum (FBS, Thermo Scientific), 100 units/mL penicillin and 0.1 mg/mL streptomycin (Gibco) in a humidified 37° C. environment with 5% CO₂. HepG2-OS8 cells, which express the single chain variable fragment (scFv) of an anti-human CD3 mAb OKT3 fused to the C-terminal domain (113-220) of mouse CD8α which includes hinge, transmembrane 3% and cytoplasmic domains, were maintained in MEM medium (Gibco) supplemented with 10% fetal bovine serum (FBS, Thermo Scientific), 100 units/mL penicillin and 0.1 mg/mL streptomycin (Gibco) in a humidified 37° C. environment with 5% CO₂.

Jurkat cells were infected with the lentivirus expressing spCas9 and sgRNA targeting human DGKα or DGKζ. Cell clones that stably knockout with DGKα/ζ (were established and maintained in the RPMI 1640 complete medium. Knockout efficiency of eSPCas9-Lenticrispr DGKα or DGKζ sgRNA in single cell clone was determined using genomic sequencing and immunoblotting method. Selected compounds did not induce DGKα or DGKζ independent IL-2 production in DGKα/ζ KO jurkat cells.

### Non-Stimulated Phosphorylated ERK Detection Assay

Cellular non-stimulated phospho-ERK were measured using a AlphaLISA-based method (Beaudet, Lucille, et al. Nature Methods. 2008, 5.12: an8-an9). Jurkat cells were subcultured in T75 flasks. The next day, growth medium was replaced to serum free RPMI 1640 for 4 hours or overnight. The cells were then seeded into 96-well plates and treated with compounds. After 2 h compound treatment, lysis buffer (PerkinElmer) was added to each well. Plates were then incubated at room temperature with shaking for 30 minutes. A total of 10 μL of cell lysate from each well of a 96-well plate was transferred to a 384-well white assay plate. Phosphor-ERK was quantitated using the AlphaLISA kit (Cat #ALSU-PERK-A10K) as described by the manufacturer manual (PerkinElmer). AlphaLISA signals were measured using a PHERAstar FSX reader (BMG Labtech). Selected compounds did not elevate ERK phosphorylation in Jurkat cells without TCR activation.

### IL-2 Production Assay in Human PBMC

Frozen human PBMC were thawed in RPMI 1640 medium and incubated at 37° C. overnight. OS8 overexpressing HepG2 cells were seeded into 384-well plates overnight. The next day, PBMC were added into the 384-well plates and then treated with compounds. PBMC and HepG2-OS8 cells were co-cultured for 48 hours at 37° C. Culture supernatant was collected for subsequent measurement of IL-2 concentration by a TR-FRET-based method (Degorce, François, et al. Current chemical genomics. 2009, 3: 22) as described by the manufacturer manual (Cisbio). FRET signals were measured using a PHERAstar FSX reader (BMG Labtech). Selected compounds showed good potency in Human PBMC assay.

### Explorative Acute Toxicity Study in BALB/c Mice

The test article was dissolved in vehicle formulation (DMA: 30% solutol HS-15(w/v): saline=20:20:60) and injected through tail vein into BALB/c mice at the doses of 2 and/or 10 mg/kg. Continuous clinical observation within 2 hours post injection was performed. Selected compounds were well tolerant at the doses of 2 and/or 10 mg/kg.

It is to be understood that, if any prior art publication is referred to herein, such reference does not constitute an admission that the publication forms a part of the common general knowledge in the art in any country.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

The disclosures of all publications, patents, patent applications and published patent applications referred to herein by an identifying citation are hereby incorporated herein by reference in their entirety.

1. A compound of formula (I), 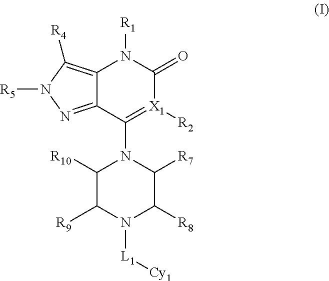
or a stereoisomer or a pharmaceutically acceptable salt thereof
wherein
X₁ is C or N,
R₁ is hydrogen, or alkyl optionally substituted with deuterium, halogen, hydroxy, alkoxy or cycloalkyl;
R₂ is hydrogen, halogen, alkyl or cyano, provided that R₂ is absent when X₁ is N;
R₄ is hydrogen, halogen, or alkyl, wherein the alkyl is optionally substituted with deuterium or halogen;
R₅ is hydrogen, alkyl, alkenyl, alkynyl, cycloalkyl, cyano, or heterocyclyl, wherein said alkyl or alkenyl is unsubstituted or substituted with halogen, cyano, heterocyclyl, alkoxy, hydroxy, cycloalkyl;
each of R₇, R₉, R₈, and R₁₀ is independently hydrogen, alkyl, alkoxy, wherein said alkyl is unsubstituted or substituted with halogen, provided that at least one of R₇ and R₉ is not hydrogen;
L₁ is a direct bond, or —C(R_{L1})(R_{L2}), wherein said each of R_{L1}, R_{L2} is independently hydrogen or C₁₋₄alkyl optionally substituted with deuterium, halogen, alkyl, alkylene, alkynyl, cyano;
Cy₁ is aryl, heterocyclyl, heteroaryl, or cycloalkyl, each of which is unsubstituted or substituted with one, two or three substituents R₃ₐ, wherein each R₃ₐ is independently selected from hydroxy, alkoxy, alkyl, halogen, aminoalkyl, R_{3b}—SO₂—, cycloalkyl, cyano, R_{3b}—C(O)—N(R_{3c})—, N(R_{3b}R_{3c})—C(O)—, N(R_{3b}R_{3c})—, R_{3b}—O—C(O)—, cycloalkyl, heterocyclyl or heterocyclyloxy, each alkyl moiety of which is unsubstituted or substituted with deuterium, halogen, alkoxy, hydroxy, cyano or heterocyclyl; wherein said cycloalkyl or heterocyclyl is unsubstituted or substituted with alkoxy, alkyl, halogen, or hydroxy, wherein R_{3b} and R_{3c} are each independently hydrogen or alkyl, or R_{3b} and R_{3b} together form a bridge containing at least one —CH₂— moiety; and
provided that the compound is not 7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 7-((2S,5R)-2,5-dimethyl-4-(1-(quinoxalin-6-yl)ethyl)piperazin-1-yl)-2,4-dimethyl-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-4-methyl-2-(2,2,2-trifluoroethyl)-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-2-(2-methoxyethyl)-4-methyl-2,4-dihydro-5H-pyrazolo[4,3-b]pyridin-5-one, 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)acetamide, methyl 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)acetate, 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)propanenitrile, 7-((2S,5R)-2,5-dimethyl-4-(1-(quinoxalin-6-yl)ethyl)piperazin-1-yl)-2-(2-hydroxyethyl)-4-methyl-2,4-dihydro-5H-pyrazolo[4,3-b]pyridin-5-one, 2-(cyclopropylmethyl)-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-2-(2-(dimethylamino)ethyl)-4-methyl-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-4-methyl-2-(oxiran-2-ylmethyl)-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-4-methyl-2-(methylsulfonyl)-2H-pyrazolo[4,3-b]pyridin-5 (4H)-one, 2-cyclobutyl-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 2-(cyclopropanecarbonyl)-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 1-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)cyclopropane-1-carbonitrile, 2-cyclopropyl-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-2-isopropyl-4-methyl-2,4-dihydro-5H-pyrazolo[4,3-b]pyridin-5-one, 2-(3-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)-1-(ethylsulfonyl)azetidin-3-yl)acetonitrile, 3-cyclopentyl-3-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-b]pyridin-2-yl)propanenitrile, 7-((2S,5R)-2,5-dimethyl-4-(1-(quinoxalin-6-yl)ethyl)piperazin-1-yl)-4-methyl-2-(prop-2-yn-1-yl)-2,4-dihydro-5H-pyrazolo[4,3-b]pyridin-5-one, 2-(but-2-yn-1-yl)-7-((2S,5R)-2,5-dimethyl-4-(1-(quinoxalin-6-yl)ethyl)piperazin-1-yl)-4-methyl-2,4-dihydro-5H-pyrazolo[4,3-b]pyridin-5-one, 2-(but-2-yn-1-yl)-7-((2S,5R)-2,5-diethyl-4-(1-(quinoxalin-6-yl)ethyl)piperazin-1-yl)-4-methyl-2,4-dihydro-5H-pyrazolo[4,3-b]pyridin-5-one, 7-((2S,5R)-2,5-dimethyl-4-(1-(quinoxalin-6-yl)ethyl)piperazin-1-yl)-4-methyl-2-(prop-1-en-2-yl)-2,4-dihydro-5H-pyrazolo[4,3-b]pyridin-5-one, 7-((2S,5R)-2,5-diethyl-4-(1-(quinoxalin-6-yl)ethyl)piperazin-1-yl)-4-methyl-2-(prop-1-en-2-yl)-2,4-dihydro-5H-pyrazolo[4,3-b]pyridin-5-one, 7-((2S,5R)-2,5-dimethyl-4-(4-(trifluoromethoxy)benzyl)piperazin-1-yl)-4-methyl-2-(prop-1-en-2-yl)-2,4-dihydro-5H-pyrazolo[4,3-b]pyridin-5-one, 7-((2S,5R)-2,5-diethyl-4-(4-(trifluoromethoxy)benzyl)piperazin-1-yl)-4-methyl-2-(prop-1-en-2-yl)-2,4-dihydro-5H-pyrazolo[4,3-b]pyridin-5-one, 2-(7-((2S,5R)-2,5-diethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(methoxymethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-3-(methoxymethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-(methoxymethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,6-difluoro-4-methoxyphenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(5-fluoropyridin-2-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-([1,2,4]triazolo[1,5-a]pyridin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,5-difluoro-4-methoxyphenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-cyclopropylphenyl)propyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-phenylethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-methoxyphenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(isoquinolin-7-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(quinolin-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-methylbenzo[d]oxazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-methylbenzo[d]oxazol-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(quinoxalin-6-ylmethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-methylbenzo[d]oxazol-4-yl)ethyl)piperazin-1-yl)-4-methyl-5-oxo-4,5-dihydro-2H-pyrazolo[4,3-b]pyridin-2-yl)acetonitrile, mixture of 2-(7-((2S,5R)-4-(1-(3,3-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile, and 2-(7-((2S,5R)-4-(1-(2,2-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-4-ethyl-5-oxo-4,5-dihydro-2H-pyrazolo[4,3-b]pyridin-2-yl)acetonitrile, 2-(7-((2S,5R)-2,5-diethyl-4-(1-(isoquinolin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-([1,2,4]triazolo[1,5-a]pyridin-7-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(naphthalen-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-4-(methyl-d3)-5-oxo-4,5-dihydro-2H-pyrazolo[4,3-b]pyridin-2-yl)acetonitrile, 2-(7-((2S,5R)-4-(1-(benzo[d]thiazol-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-((4-fluoro-2-methoxyphenyl)(4-fluorophenyl)methyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(quinoxalin-6-ylmethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinolin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinolin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(isoquinolin-3-yl)ethyl)piperazin-1-yl)-4-methyl-5-oxo-4,5-dihydro-2H-pyrazolo[4,3-b]pyridine-2-yl)acetonitrile, 2-(7-((2S,5R)-2,5-diethyl-4-(1-(1-methyl-1H-benzo[d]imidazol-2-yl)ethyl)piperazin-1-yl)-4-methyl-5-oxo-4,5-dihydro-2H-pyrazolo[4,3-b]pyridine-2-yl)acetonitrile, 2-(7-((2S,5R)-2,5-diethyl-4-(1-(quinolin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinolin-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(isoquinolin-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(1,8-naphthyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-methyl-1H-benzo[d]imidazol-6-yl)ethyl)piperazin-1-yl)-4-methyl-5-oxo-4,5-dihydro-2H-pyrazolo[4,3-b]pyridin-2-yl)acetonitrile, mixture of 2-(7-((2S,5R)-2,5-diethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-diethyl-4-(1-(2-methylquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,2-difluorobenzo[d][1,3]dioxol-4-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluorophenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyridine-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-fluoro-4-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(4-fluorobenzyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-methoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-cyclopropyl-2-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-fluoro-4-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-fluoropyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(2,4-difluorobenzyl)-2,5-diethylpiperazin-1-yl)-4-methyl-5-oxo-4,5-dihydro-2H-pyrazolo[4,3-b]pyridine-2-yl)acetonitrile, 2-(7-((2S,5R)-4-(1-(4-cyclopropyl-2-fluorophenyl)propyl)-2,5-diethylpiperazin-1-yl)-4-methyl-5-oxo-4,5-dihydro-2H-pyrazolo[4,3-b]pyridine-2-yl)acetonitrile, 2-(7-((2S,5R)-2,5-diethyl-4-(4-(trifluoromethoxy)benzyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methoxy-4-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(methoxymethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-(trifluoromethoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-isopropoxypyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-3-methoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,6-difluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-methylphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(2-methoxyethoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyridin-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(3,5-difluoropyridin-4-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyrazin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-phenylethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methoxypyridin-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methoxypyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-(methoxymethyl)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2,6-dimethoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-4-(2-hydroxyethyl)-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-4-(²-methoxyethyl)-5-oxo-4,5-dihydro-2H-pyrazolo[4,3-b]pyridin-2-yl)acetonitrile, 2-(7-((2S,5R)-5-ethyl-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2-ethyl-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5S)-5-(hydroxymethyl)-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5S)-5-(methoxymethyl)-2-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2R,5R)-2-(hydroxymethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(quinoxalin-6-yl)ethyl)piperazin-1-yl)-5-oxo-4,5-dihydro-2H-pyrazolo[4,3-b]pyridin-2-yl)acetonitrile, 2-(7-((2S,5R)-2,5-diethyl-4-(1-(4-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-methyl-2-oxo-1,2-dihydropyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methoxypyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-(methoxymethyl)phenyl)ethyl)piperazin-1-yl)-4-methyl-5-oxo-4,5-dihydro-2H-pyrazolo[4,3-b]pyridin-2-yl)acetonitrile, 3-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)benzamide, N-(3-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)phenyl)acetamide, 2-(7-((2S,5R)-2,5-diethyl-4-(1-(3-methyl-2-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-bromo-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(methylsulfonyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-fluoro-4-(trifluoromethyl)pyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-methyl-2-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-methyl-2-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-chloro-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methyl-6-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-(trifluoromethyl)pyridin-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-5-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile, 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)benzamide, 2-(7-((2S,5R)-2,5-diethyl-4-(1-(3-(hydroxymethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(trifluoromethoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-5-oxo-4-(2,2,2-trifluoroethyl)-4,5-dihydro-2H-pyrazolo[4,3-b]pyridin-2-yl)acetonitrile, 2-(4-methyl-5-oxo-7-(5-(1-(quinoxalin-6-yl)ethyl)-2,5-diazabicyclo[2.2.2]octan-2-yl)-4,5-dihydro-2H-pyrazolo[4,3-b]pyridin-2-yl)acetonitrile, 2-(7-((2S,5R)-4-(1-(2,3-dihydrobenzo[b][1,4]dioxin-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(p-tolyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-(difluoromethyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-((S)-1-(quinoxalin-6-yl)ethyl)piperazin-1-yl)-5-oxo-4,5-dihydro-2H-pyrazolo[4,3-b]pyridin-2-yl)acetonitrile, 2-(7-((2S,5R)-2,5-diethyl-4-(1-(pyrimidin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyrimidin-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,4-difluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(6-chloro-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile, 2-(cyanomethyl)-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridine-6-carbonitrile, 2-(7-((2S,5R)-2,5-diethyl-4-(1-(6-(trifluoromethyl)pyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-fluoro-3-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-4-(1-(4-fluoro-2-methoxyphenyl)ethyl)-2-methylpiperazin-1-yl)-4-methyl-5-oxo-4,5-dihydro-2H-pyrazolo[4,3-b]pyridin-2-yl)acetonitrile, 2-(7-((2S,5R)-4-(1-(3,4-dihydro-2H-benzo[b][1,4]oxazin-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(quinoxalin-6-yl)ethyl)piperazin-1-yl)-4-(2,2-difluoroethyl)-5-oxo-4,5-dihydro-2H-pyrazolo[4,3-b]pyridin-2-yl)acetonitrile, 2-(7-((2S,5R)-2,5-diethyl-4-(1-(5-isopropoxypyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-ethoxy-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-isopropoxyphenyl)ethyl)piperazin-1-yl)-4-methyl-5-oxo-4,5-dihydro-2H-pyrazolo[4,3-b]pyridin-2-yl)acetonitrile, 2-(7-((2R,5R)-2-(methoxymethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2-(2-hydroxyethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrilecetonitrile, 2-(7-((2S,5R)-2,5-diethyl-4-(1,2,3,4-tetrahydronaphthalen-1-yl)piperazin-1-yl)-4-methyl-5-oxo-4,5-dihydro-2H-pyrazolo[4,3-b]pyridin-2-yl)acetonitrile, 2-(7-((2S,5R)-4-(1-(3,5-difluoropyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(2,3-dihydro-1H-inden-1-yl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(chroman-4-yl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-((dimethylamino)methyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(hydroxymethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-methoxyphenyl)ethyl)piperazin-1-yl)-4-(2-hydroxyethyl)-5-oxo-4,5-dihydro-2H-pyrazolo[4,3-b]pyridin-2-yl)acetonitrile, 2-(7-((2S,5R)-2,5-dimethyl-4-(1-(quinoxalin-6-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(quinoxalin-2-yl)ethyl)piperazin-1-yl)-4-methyl-5-oxo-4,5-dihydro-2H-pyrazolo[4,3-b]pyridin-2-yl)acetonitrile, 2-(cyanomethyl)-7-((2S,5R)-5-ethyl-2-methyl-4-(1-(quinoxalin-6-yl)ethyl)piperazin-1-yl)-4-methyl-5-oxo-4,5-dihydro-2H-pyrazolo[4,3-b]pyridine-6-carbonitrile, 2-(7-((2S,5R)-2,5-diethyl-4-(1-(2-methoxy-4-(trifluoromethyl)phenyl)ethyl)piperazin-1-yl)-4-methyl-5-oxo-4,5-dihydro-2H-pyrazolo[4,3-b]pyridin-2-yl)acetonitrile, 2-((2S,5R)-1-(2-(cyanomethyl)-4-methyl-5-oxo-4,5-dihydro-2H-pyrazolo[4,3-b]pyridin-7-yl)-5-methyl-4-(1-(quinoxalin-6-yl)ethyl)piperazin-2-yl)acetonitrile, 2-(7-((2S,5R)-4-(1-(2,3-dihydro-[1,4]dioxino[2,3-b]pyridin-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-isopropoxyphenyl)ethyl)piperazin-1-yl)-4-methyl-5-oxo-4,5-dihydro-2H-pyrazolo[4,3-b]pyridin-2-yl)acetonitrile, 2-(7-((3R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(6-chloro-7-((3R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(methylamino)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2-(2-methoxyethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, N-(2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)phenyl)acetamide, 2-(7-((2S,5R)-2,5-diethyl-4-(1-(1-methyl-2,3-dihydro-1H-pyrido[2,3-b][1,4]oxazin-6-yl)ethyl)piperazin-1-yl)-4-methyl-5-oxo-4,5-dihydro-2H-pyrazolo[4,3-b]pyridin-2-yl)acetonitrile, 2-(4-(cyclopropylmethyl)-7-((2S,5R)-2,5-diethyl-4-(1-(quinoxalin-6-yl)ethyl)piperazin-1-yl)-5-oxo-4,5-dihydro-2H-pyrazolo[4,3-b]pyridin-2-yl)acetonitrile, 2-(7-((2S,5R)-2,5-dimethyl-4-(1-(quinoxalin-6-yl)ethyl)piperazin-1-yl)-6-fluoro-4-methyl-5-oxo-4,5-dihydro-2H-pyrazolo[4,3-b]pyridin-2-yl)acetonitrile, 2-(cyanomethyl)-7-((2S,5R)-3-ethyl-4-(1-(quinoxalin-6-yl)ethyl)piperazin-1-yl)-4-methyl-5-oxo-4,5-dihydro-2H-pyrazolo[4,3-b]pyridine-6-carbonitrile, 2-(7-((2S,5R)-2,5-diethyl-4-(1-(4-fluoro-2-(trifluoromethoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-(difluoromethoxy)-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(difluoromethyl)-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, methyl 4-(2-(cyanomethyl)-4-methyl-5-oxo-4,5-dihydro-2H-pyrazolo[4,3-b]pyridin-7-yl)-1-(1-(quinoxalin-6-yl)ethyl)piperazine-2-carboxylate, 2-(7-((2S,5R)-2,5-diethyl-4-(1-(1-ethyl-1H-imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-cyclopropyl-4-fluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-1H-indol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(quinoxalin-6-yl)ethyl)piperazin-1-yl)-5-oxo-4,5-dihydro-2H-pyrazolo[4,3-b]pyridin-2-yl)acetonitrile, 2-(4-methyl-7-((3S)-3-methyl-4-(1-(quinoxalin-6-yl)ethyl)piperazin-1-yl)-5-oxo-4,5-dihydro-2H-pyrazolo[4,3-b]pyridin-2-yl)acetonitrile, 2-(6-bromo-7-((2S,5R)-3-ethyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-(difluoromethoxy)pyridin-3-yl)ethyl)-2,5-diethylpiperazin-1-yl)-4-methyl-5-oxo-4,5-dihydro-2H-pyrazolo[4,3-b]pyridin-2-yl)acetonitrile, mixture of 2-(7-((2S,5R)-2,5-diethyl-4-(1-(1-ethyl-5-methyl-1H-imidazol-2-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-diethyl-4-(1-(1-ethyl-4-methyl-1H-imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-4,5,6,7-tetrahydro-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(quinoxalin-6-yl)ethyl)piperazin-1-yl)-5-oxo-4,5-dihydro-2H-pyrazolo[4,3-d]pyrimidin-2-yl)acetonitrile, 2-(7-(3-(hydroxymethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-ethyl-3H-imidazo[4,5-b]pyridin-2-yl)ethyl)piperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-2,5-diethyl-4-(1-(1-ethyl-1H-benzo[d]imidazol-2-yl)ethyl)piperazin-1-yl)-4-methyl-2,4-dihydro-5H-pyrazolo[4,3-b]pyridin-5-one, 2-(7-((2S,5R)-4-(1-(5-chloro-1-ethyl-1H-imidazol-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(1-hydroxyethyl)phenyl)ethyl)piperazin-1-yl)-4-methyl-5-oxo-4,5-dihydro-2H-pyrazolo[4,3-b]pyridin-2-yl)acetonitrile, 2,2′-(6-cyano-7-((2S,5R)-5-ethyl-2-methyl-4-(1-(quinoxalin-6-yl)ethyl)piperazin-1-yl)-5-oxo-2H-pyrazolo[4,3-b]pyridine-2,4 (5H)-diyl)diacetonitrile, 2-(7-((2S,5R)-2,5-diethyl-4-(1-(quinoxalin-6-yl)ethyl)piperazin-1-yl)-4-isopropyl-5-oxo-4,5-dihydro-2H-pyrazolo[4,3-b]pyridin-2-yl)acetonitrile, 2-(7-((2S,5R)-4-(1-(4-chloro-1-ethyl-1H-imidazol-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-propyl-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-isopropyl-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(quinoxalin-6-yl)ethyl)piperazin-1-yl)-6-fluoro-5-oxo-4,5-dihydro-2H-pyrazolo[4,3-b]pyridin-2-yl)acetonitrile, 2-(7-((2S,5R)-4-(1-(2-fluoro-4-(trifluoromethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-(trifluoromethyl)phenyl)ethyl)piperazin-1-yl)-4-methyl-5-oxo-4,5-dihydro-2H-pyrazolo[4,3-b]pyridin-2-yl)acetonitrile, 4-(2-(cyanomethyl)-4-methyl-5-oxo-4,5-dihydro-2H-pyrazolo[4,3-b]pyridin-7-yl)-1-(1-(quinoxalin-6-yl)ethyl)piperazine-2-carboxamide, 2-(7-((2S,5R)-4-(1-(4-cyclopropyl-2-methoxyphenyl)ethyl)-2,5-diethylpiperazin-1-yl)-4-methyl-5-oxo-4,5-dihydro-2H-pyrazolo[4,3-b]pyridin-2-yl)acetonitrile, 2-(cyanomethyl)-7-((2S,5R)-5-ethyl-2-methyl-4-(1-(quinoxalin-6-yl)ethyl)piperazin-1-yl)-5-oxo-4,5-dihydro-2H-pyrazolo[4,3-b]pyridine-6-carbonitrile, 2-(7-((2S,5R)-4-(1-(2,4-difluoro-6-methoxyphenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-5-ethyl-4-(1-(2-fluoro-4-(trifluoromethyl)phenyl)ethyl)-2-methylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-5-(trifluoromethyl)-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-6-(trifluoromethyl)-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-ethyl-4-fluorophenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(3-(trifluoromethyl)benzoyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-6-oxo-1,6-dihydropyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-2-methyl-1H-imidazol-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(quinoxalin-6-yl)ethyl)piperazin-1-yl)-3,4-dimethyl-5-oxo-4,5-dihydro-2H-pyrazolo[4,3-b]pyridin-2-yl)acetonitrile, 2-(7-((2S,5R)-2,5-diethyl-4-(1-(1-ethyl-3-(trifluoromethyl)-1H-pyrazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-3-(trifluoromethyl)-1H-pyrazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(3-chloro-1-ethyl-1H-pyrazol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-ethyl-1-methyl-1H-pyrazol-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-(2-hydroxyethyl)-1H-benzo[d]imidazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-1H-imidazo[4,5-b]pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-4-methyl-1H-pyrazol-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-(trifluoromethyl)pyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-4-(1-(4-fluoro-2-(trifluoromethyl)phenyl)ethyl)-2-methylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-2-methyl-1H-imidazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-d]pyrimidin-2-yl)acetonitrile, or 2-(7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-d]pyrimidin-2-yl)acetonitrile, 2-ethyl-7-((2S,5R)-5-ethyl-2-methyl-4-(1-(quinoxalin-6-yl)ethyl)piperazin-1-yl)-4-methyl-2,4-dihydro-5H-pyrazolo[4,3-b]pyridin-5-one, 2-(2,2-difluoroethyl)-7-((2S,5R)-5-ethyl-2-methyl-4-(1-(quinoxalin-6-yl)ethyl)piperazin-1-yl)-4-methyl-2,4-dihydro-5H-pyrazolo[4,3-b]pyridin-5-one, 2-(7-((2S,5R)-4-(1-cyclohexylethyl)-2,5-dimethylpiperazin-1-yl)-4-methyl-5-oxo-4,5-dihydro-2H-pyrazolo[4,3-b]pyridin-2-yl)acetonitrile, 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluorobenzonitrile, 2-(7-((2R,5R)-5-ethyl-2-(methoxymethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-chloro-4-(trifluoromethyl)pyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-4-methyl-1H-pyrazol-5-yl)ethyl)piperazin-1-yl)-4-methyl-5-oxo-4,5-dihydro-2H-pyrazolo[4,3-b]pyridin-2-yl)acetonitrile, 7-((2S,5R)-4-(1-(1-(2,2-difluoroethyl)-1H-pyrazol-5-yl)ethyl)-2,5-diethylpiperazin-1-yl)-4-methyl-2,4-dihydro-5H-pyrazolo[4,3-b]pyridin-5-one, 2-(7-((2R,5R)-5-ethyl-2-(hydroxymethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-ethyl-7-(trifluoromethyl)-1 H-benzo[d]imidazol-2-yl)ethyl)piperazin-1-yl)-4-methyl-5-oxo-4,5-dihydro-2H-pyrazolo[4,3-b]pyridin-2-yl)acetonitrile, 7-((2S,5R)-2,5-diethyl-4-(1-(2-fluoro-4-(trifluoromethyl)phenyl)ethyl)piperazin-1-yl)-4-methyl-2,4-dihydro-5H-pyrazolo[4,3-b]pyridin-5-one, 2-(7-((2S,5R)-2,5-diethyl-4-(1-(1-methyl-3-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-methyl-5-(trifluoromethyl)-1H-pyrazol-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5-(difluoromethyl)-1-methyl-1H-pyrazol-4-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-methyl-4-(trifluoromethyl)pyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(trifluoromethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(1-(2,2-difluoroethyl)-1 H-imidazol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-2,5-diethyl-4-(1-(4-fluoro-2-(trifluoromethyl)phenyl)ethyl)piperazin-1-yl)-4-methyl-2,4-dihydro-5H-pyrazolo[4,3-b]pyridin-5-one, 2-(7-((2S,5R)-2,5-diethyl-4-(1-(1-ethyl-2-(trifluoromethyl)-1H-imidazol-5-yl)ethyl)piperazin-1-yl)-4-methyl-5-oxo-4,5-dihydro-2H-pyrazolo[4,3-b]pyridin-2-yl)acetonitrile, 3-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-1-ethyl-1H-pyrazole-4-carbonitrile, 2-(7-((2S,5R)-2,5-diethyl-4-(4-fluoro-2-(trifluoromethyl)benzyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(tetrahydro-2H-pyran-4-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(quinoxalin-6-yl)ethyl)piperazin-1-yl)-4-methyl-5-oxo-3-(trifluoromethyl)-4,5-dihydro-2H-pyrazolo[4,3-b]pyridin-2-yl)acetonitrile or 2-(7-((2S,5R)-2,5-dimethyl-4-((R)-1-(quinoxalin-6-yl)ethyl)piperazin-1-yl)-4-methyl-5-oxo-3-(trifluoromethyl)-4,5-dihydro-2H-pyrazolo[4,3-b]pyridin-2-yl)acetonitrile, 2-(7-((2S,5R)-2,5-dimethyl-4-((S)-1-(quinoxalin-6-yl)ethyl)piperazin-1-yl)-4-methyl-5-oxo-3-(trifluoromethyl)-4,5-dihydro-1H-pyrazolo[4,3-b]pyridin-1-yl)acetonitrile or 2-(7-((2S,5R)-2,5-dimethyl-4-((R)-1-(quinoxalin-6-yl)ethyl)piperazin-1-yl)-4-methyl-5-oxo-3-(trifluoromethyl)-4,5-dihydro-1H-pyrazolo[4,3-b]pyridin-1-yl)acetonitrile, 2-(3-chloro-7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-d]pyrimidin-2-yl)acetonitrile, or 2-(3-chloro-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-4-(4-fluoro-2-(trifluoromethyl)benzyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(difluoromethoxy)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-omethoxy-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(trifluoromethoxy)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,3-dihydro-[1,4]dioxino[2,3-b]pyridin-7-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-chloro-2-(trifluoromethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(difluoromethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(1,1-difluoroethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(quinoxalin-6-yl)propyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(trifluoromethyl)phenyl)propyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(trifluoromethyl)phenyl)ethyl)-2,5-dimethylpiperazin-1-yl)-4-(methyl-d3)-5-oxo-4,5-dihydro-2H-pyrazolo[4,3-b]pyridin-2-yl)acetonitrile, 2-(7-((2S,5R)-2,5-dimethyl-4-((R)-1-(quinoxalin-6-yl)ethyl)piperazin-1-yl)-3-fluoro-4-methyl-5-oxo-4,5-dihydro-1H-pyrazolo[4,3-b]pyridin-1-yl)acetonitrile , or 2-(7-((2S,5R)-2,5-dimethyl-4-((S)-1-(quinoxalin-6-yl)ethyl)piperazin-1-yl)-3-fluoro-4-methyl-5-oxo-4,5-dihydro-1H-pyrazolo[4,3-b]pyridin-1-yl)acetonitrile, 2-(7-((2R,5R)-5-ethyl-2-((R)-1-hydroxyethyl)-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((R)-1-(quinoxalin-6-yl)ethyl)piperazin-1-yl)-5-oxo-4,5-dihydro-2H-pyrazolo[4,3-b]pyridin-2-yl)acetonitrile, or 2-(7-((2S,5R)-2,5-dimethyl-4-((S)-1-(quinoxalin-6-yl)ethyl)piperazin-1-yl)-5-oxo-4,5-dihydro-2H-pyrazolo[4,3-b]pyridin-2-yl)acetonitrile, 2-(7-((2R,5R)-2-(difluoromethyl)-5-methyl-4-(1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(2,2-difluoro-1-(quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(3-chloro-7-((2S,5R)-2,5-dimethyl-4-((R)-1-(quinoxalin-6-yl)ethyl)piperazin-1-yl)-4-methyl-5-oxo-4,5-dihydro-2H-pyrazolo[4,3-b]pyridin-2-yl)acetonitrile, or 2-(3-chloro-7-((2S,5R)-2,5-dimethyl-4-((S)-1-(quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(quinoxalin-6-yl)ethyl)piperazin-1-yl)-3-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(trifluoromethyl)phenyl)ethyl)piperazin-1-yl)-5-oxo-4,5-dihydro-2H-pyrazolo[4,3-b]pyridin-2-yl)acetonitrile, 2-(7-((2S,5R)-4-(1-(4-fluoro-2-(trifluoromethyl)phenyl)ethyl)-2,5-dimethylpiperazin-1-yl)-4-methyl-5-oxo-4,5-dihydro-2H-pyrazolo[4,3-d]pyrimidin-2-yl)acetonitrile, 2-(7-((2S,5R)-4-(1-(4-fluoro-2-(trifluoromethyl)phenyl)ethyl)-2,5-dimethylpiperazin-1-yl)-3,4-dimethyl-5-oxo-4,5-dihydro-2H-pyrazolo[4,3-d]pyrimidin-2-yl)acetonitrile, 2-(7-((2S,5R)-2,5-dimethyl-4-(1-(quinoxalin-6-yl-2,3-d2)ethyl)piperazin-1-yl)-4-methyl-5-oxo-4,5-dihydro-2H-pyrazolo[4,3-b]pyridin-2-yl)acetonitrile, mixture of 2-(7-((2S,5R)-2,5-dimethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile and 2-(7-((2S,5R)-2,5-dimethyl-4-(1-(2-methylquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(2-methylquinoxalin-6-yl)ethyl)piperazin-1-yl)-4-methyl-5-oxo-4,5-dihydro-2H-pyrazolo[4,3-b]412yridine-2-yl)acetonitrile, 2-(7-((2S,5R)-2,5-dimethyl-4-((R)-1-(2-methylquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(3-methylquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(3-methoxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(3-hydroxyoxetan-3-yl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,6-dichloro-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3,4-difluoro-2-methoxyphenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(quinoxalin-6-yl)ethyl)piperazin-1-yl)-6-methoxy-4-methyl-5-oxo-4,5-dihydro-2H-pyrazolo[4,3-b]pyridin-2-yl)acetonitrile, 7-((2S,5R)-2,5-diethyl-4-(1-(4-fluoro-2-(trifluoromethyl)phenyl)ethyl)piperazin-1-yl)-4-methyl-2,4-dihydro-5H-pyrazolo[4,3-b]pyridin-5-one, 2-(7-((2S,5R)-2,5-diethyl-4-(1-(4-fluoro-2-(trifluoromethyl)phenyl)ethyl)piperazin-1-yl)-4-(methyl-d3)-5-oxo-4,5-dihydro-2H-pyrazolo[4,3-b]pyridin-2-yl)acetonitrile, 2-(7-((2S,5R)-4-(1-(4-chloro-2-(trifluoromethyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-ethylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-hydroxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-chloroquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methoxyquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-hydroxyquinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-methoxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-hydroxyquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(dimethylphosphoryl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(1-methoxyethyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-(cyanomethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3,4-difluoro-2-(trifluoromethyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluorobenzamide, methyl 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluorobenzoate, 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluoro-N-methylbenzamide, 2-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-5-fluoro-N,N-dimethylbenzamide, 2-(7-((2S,5R)-2,5-diethyl-4-(1-(4-fluoro-2-(trifluoromethyl)phenyl)propyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,3-dimethylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((3R,4R)-3-ethoxy-4-(3-(trifluoromethyl)phenoxy)piperidin-1-yl)-4-methyl-5-oxo-4,5-dihydro-2H-pyrazolo[4,3-b]pyridin-2-yl)acetonitrile, 2-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)-N-(2-methoxyethyl)-2-(4-(trifluoromethyl)phenyl)acetamide, 2-(7-((2S,5R)-4-(2-(4-fluorophenyl)propan-2-yl)-2,5-dimethylpiperazin-1-yl)-4-methyl-5-oxo-4,5-dihydro-2H-pyrazolo[4,3-b]pyridin-2-yl)acetonitrile, 2-(7-(2,5-dimethyl-4-(methyl(3-methylquinoxalin-6-yl)amino)piperidin-1-yl)-4-methyl-5-oxo-4,5-dihydro-2H-pyrazolo[4,3-b]pyridin-2-yl)acetonitrile, 2-allyl-7-((2S,5R)-2,5-dimethyl-4-((S)-1-(quinoxalin-6-yl)ethyl)piperazin-1-yl)-4-methyl-2,4-dihydro-5H-pyrazolo[4,3-b]pyridin-5-one, or 2-allyl-7-((2S,5R)-2,5-dimethyl-4-((R)-1-(quinoxalin-6-yl)ethyl)piperazin-1-yl)-4-methyl-2,4-dihydro-5H-pyrazolo[4,3-b]pyridin-5-one, 2-(but-3-en-1-yl)-7-((2S,5R)-2,5-dimethyl-4-((S)-1-(quinoxalin-6-yl)ethyl)piperazin-1-yl)-4-methyl-2,4-dihydro-5H-pyrazolo[4,3-b]pyridin-5-one, or 2-(but-3-en-1-yl)-7-((2S,5R)-2,5-dimethyl-4-((R)-1-(quinoxalin-6-yl)ethyl)piperazin-1-yl)-4-methyl-2,4-dihydro-5H-pyrazolo[4,3-b]pyridin-5-one, 2-allyl-7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one, 2-(but-2-en-1-yl)-7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one, 2-(3,3-difluoroallyl)-7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one, 2-(7-((2S,5R)-2,5-dimethyl-4-(1-(3-methylisoquinolin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(isoquinolin-3-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-methylquinolin-7-yl)ethyl)piperazin-1-yl)-4-methyl-5-oxo-4,5-dihydro-2H-pyrazolo[4,3-b]pyridin-2-yl)acetonitrile, 7-(1-((2R,5S)-4-(2-(cyanomethyl)-4-methyl-5-oxo-4,5-dihydro-2H-pyrazolo[4,3-b]pyridin-7-yl)-2,5-dimethylpiperazin-1-yl)ethyl)-2-naphthonitrile, 2-(7-((2S,5R)-2,5-dimethyl-4-((S)-1-(3-methylquinoxalin-6-yl)ethyl)piperazin-1-yl)-5-oxo-4,5-dihydro-2H-pyrazolo[4,3-b]pyridin-2-yl)acetonitrile, 2-(7-((2S,5R)-4-(1-(3-(hydroxymethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-cyclopropylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-(difluoromethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3-(1,1-difluoroethyl)quinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(3-(methyl-d3)quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(3-(trifluoromethyl)quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-(trifluoromethyl)quinoxalin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-hydroxy-3-methylquinoxalin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-((S)-1-(3-methylquinoxalin-6-yl)ethyl)piperazin-1-yl)-6-fluoro-4-methyl-5-oxo-4,5-dihydro-2H-pyrazolo[4,3-b]pyridin-2-yl)acetonitrile, 2-(cyanomethyl)-7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-b]pyridine-6-carbonitrile, 2-(7-((2S,5R)-2,5-dimethyl-4-((S)-1-(3-methylquinoxalin-6-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-dimethyl-4-(1-(2-methylbenzo[d]thiazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-methyl-[1,2,4]triazolo[1,5-a]pyridin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(benzo[d]thiazol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(3H-spiro[benzo[b][1,4]dioxine-2,1′-cyclopropan]-7-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,3-dihydro-[1,4]dioxino[2,3-b]pyridin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(3,3-dimethyl-2,3-dihydro-[1,4]dioxino[2,3-b]pyridin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,2-dimethyl-2,3-dihydro-[1,4]dioxino[2,3-b]pyridin-7-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2-(1-methoxyethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-(1-(difluoromethoxy)ethyl)-4-fluorophenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2-(1-methoxycyclopropyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(3-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4,4-difluorochroman-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-5-ethyl-4-((4-fluorophenyl)(5-(trifluoromethyl)pyridin-2-yl)methyl)-2-methylpiperazin-1-yl)-4-methyl-5-oxo-4,5-dihydro-2H-pyrazolo[4,3-b]pyridin-2-yl)acetonitrile, 2-(7-((2S,5R)-4-(di-p-tolylmethyl)-2,5-dimethylpiperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-2,5-diethyl-4-(1-(2-fluoro-4-(trifluoromethyl)phenyl)ethyl)piperazin-1-yl)-4-methyl-2,4-dihydro-5H-pyrazolo[4,3-b]pyridin-5-one, 2-(but-2-yn-1-yl)-7-((2S,5R)-4-(1-(3-(difluoromethyl)quinoxalin-6-yl)ethyl)-2,5-dimethylpiperazin-1-yl)-4-methyl-2,4-dihydro-5H-pyrazolo[4,3-b]pyridin-5-one, 2-(but-2-yn-1-yl)-7-((2S,5R)-2,5-dimethyl-4-((S)-1-(3-methylquinoxalin-6-yl)ethyl)piperazin-1-yl)-4-methyl-2,4-dihydro-5H-pyrazolo[4,3-b]pyridin-5-one, 2-(7-((2S,5R)-4-(1-(2,3-dihydrofuro[2,3-b]pyridin-6-yl)ethyl)-2,5-diethylpiperazin-1-yl)-4-methyl-5-oxo-4,5-dihydro-2H-pyrazolo[4,3-b]pyridin-2-yl)acetonitrile, 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)phenyl)-2-methylpropanenitrile, 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)propyl)phenyl)-2-methylpropanenitrile, 2-(7-((2S,5R)-2,5-diethyl-4-(1-(2-fluoro-4-methylphenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-((2,2-dimethylmorpholino)methyl)phenyl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-((2,2-dimethylmorpholino)methyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-(((2S,6R)-2,6-dimethylmorpholino)methyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-((4,4-difluoropiperidin-1-yl)methyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-3-fluorophenyl)-2-methylpropanenitrile, 2-(7-((2S,5R)-2,5-diethyl-4-(1-(5-methylpyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5-(difluoromethyl)pyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-methylpyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-cyclopropylpyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(6-(difluoromethyl)pyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(3,3-dimethyl-2,3-dihydrofuro[3,2-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-fluoropyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoro-5-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5,6-dimethylpyrazin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-methylpyrazin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5,6,7,8-tetrahydroquinoxalin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-6-isopropoxypyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoro-5-methylpyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5-chloropyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-(trifluoromethoxy)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoro-5-(trifluoromethoxy)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5,6-dimethylpyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-methoxy-6-methylpyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-methylpyridazin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methylisoquinolin-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-methylisoquinolin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,6-naphthyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(4-cyclopropylbenzoyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(5-methylpicolinoyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(2,6-naphthyridine-3-carbonyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(isoquinoline-3-carbonyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(quinoline-7-carbonyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(quinoxaline-2-carbonyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-((tetrahydrofuran-3-yl)oxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-((tetrahydrofuran-3-yl)methoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-methoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(oxetan-3-yloxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(oxetan-3-ylmethoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-((tetrahydro-2H-pyran-4-yl)oxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-((4,4-difluoropiperidin-1-yl)methyl)pyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-(4,4-difluoropiperidine-1-carbonyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(morpholinomethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(3,4-dimethylphenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(4,5-dimethylpyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(6-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)pyridin-3-yl)-2-methylpropanenitrile, 2-(7-((2S,5R)-2,5-diethyl-4-(1-(3-fluoro-5-isopropoxypyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(imidazo[1,2-a]pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyrazolo[1,5-a]pyridin-2-yl)ethyl)piperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-4-(1-(2,2-dimethylbenzo[d][1,3]dioxol-5-yl)ethyl)-2,5-dimethylpiperazin-1-yl)-4-methyl-2,4-dihydro-5H-pyrazolo[4,3-b]pyridin-5-one, 2-(but-2-yn-1-yl)-7-((2S,5R)-4-(1-(2,2-difluorobenzo[d][1,3]dioxol-5-yl)ethyl)-2,5-dimethylpiperazin-1-yl)-4-methyl-2,4-dihydro-5H-pyrazolo[4,3-b]pyridin-5-one, 2-(but-2-yn-1-yl)-7-((2S,5R)-4-(1-(2,2-difluorobenzo[d][1,3]dioxol-5-yl)ethyl)-5-ethyl-2-methylpiperazin-1-yl)-4-methyl-2,4-dihydro-5H-pyrazolo[4,3-b]pyridin-5-one, 2-(but-2-yn-1-yl)-7-((2S,5R)-5-ethyl-4-(1-(isoquinolin-3-yl)ethyl)-2-methylpiperazin-1-yl)-4-methyl-2,4-dihydro-5H-pyrazolo[4,3-b]pyridin-5-one, 2-(but-2-yn-1-yl)-7-((2S,5R)-5-ethyl-2-methyl-4-(1-(quinoxalin-2-yl)ethyl)piperazin-1-yl)-4-methyl-2,4-dihydro-5H-pyrazolo[4,3-b]pyridin-5-one, or 2-(but-2-yn-1-yl)-7-((2S,5R)-5-ethyl-2-methyl-4-(1-(quinolin-7-yl)ethyl)piperazin-1-yl)-4-methyl-2,4-dihydro-5H-pyrazolo[4,3-b]pyridin-5-one.

2. The compound of claim 1, wherein R₁ is hydrogen, or C₁₋₄ alkyl optionally substituted with deuterium, halogen, hydroxy, alkoxy or cycloalkyl; preferably R₁ is hydrogen, or C₁₋₃ alkyl optionally substituted with deuterium or halogen.

3. The compound of any one of claims 1-2, wherein R₁ is hydrogen, methyl, ethyl, isopropyl, n-propyl, methyl-d3; preferably R₁ is hydrogen, methyl, ethyl, methyl-d3; more preferably R₁ is methyl.

4. The compound of any one of claims 1-3, wherein R₂ is hydrogen, halogen, C₁₋₄alkyl or cyano, provided that X₁ is C; preferably R₂ is hydrogen, F, Br, Cl, methyl, ethyl or —CN; more preferably R₂ is F, Br, methyl or —CN.

5. The compound of any one of claims 1-4, wherein R₄ is hydrogen, halogen or alkyl; preferably R₄ is hydrogen.

6. The compound of any one of claims 1-5, wherein R₅ is hydrogen, alkyl, alkenyl or alkynyl, wherein said alkyl is unsubstituted or substituted with cyano, cycloalkyl or cycloalkoxy; preferably R₅ is C₁₋₆alkyl, C₂₋₆alkenyl or C₂₋₄alkynyl, wherein said alkyl is substituted with cyano, C₃₋₆cycloalkyl or C₂₋₆cycloalkoxy.

7. The compound of any one of claims 1-6, wherein R₅ is hydrogen, —CH₂—CN, —CH(CH₃)CN, —CH₂—CH₂—CN, —CH₂—CH₂—CH₂—CN, —CH(CH₃)—CH₂—CN, —CH₂—CH(CH₃)—CN, —CH(CH₂CH₃)—CN, oxiran-2-ylmethyl, oxiran-2-yl, oxetane-3-ylmethyl, oxetane-2-methyl, oxetane-3-yl, oxetane-2-yl, prop-2-yn-1-yl, but-2-yn-1-yl, but-3-yn-1-yl, pent-2-yn-1-yl, pent-3-yn-1-yl, pent-4-yn-1-yl, prop-2-en-1-yl, but-2-en-1-yl, but-3-en-1-yl, pent-2-en-1-yl, pent-3-en-1-yl, pent-4-en-1-yl, methyl, ethyl, propyl, isopropyl, cyclopropyl, cyclobutyl, azetidine-2-yl, azetidine-3-yl, azetidine-3-ylmethyl, azetidine-1-yl, azetidine-1-ylmethyl, aziridine-1-yl, aziridine-1-ylmethyl, aziridine-2-yl, aziridine-2-ylmethyl, 1-cyanocyclopropyl, 2-cyanocyclopropyl or 2-cyanocyclobutyl; preferably, R₅ is hydrogen, —CH₂—CN, —CH(CH₃)CN, —CH₂—CH₂—CN, CH₂—CH₂—CH₂—CN, —CH(CH)—CH₂—CN, —CH₂—CH(CH₃)—CN, —CH(CH₂CH₃)—CN, oxetane-3-ylmethyl, oxetane-2-methyl, oxetane-3-yl, oxetane-2-yl, prop-2-yn-1-yl, but-2-yn-1-yl, but-3-yn-1-yl or pent-3-yn-1-yl; more preferably, R₅ is CN—CH₂—, —CH₂—CH₂—CN, but-2-yn-1-yl, but-2-yn-1-yl, or oxetane-3-ylmethyl, oxetane-3-yl.

8. The compound of any one of claims 1-7, wherein each of R₇ and R₉ is independently hydrogen, alkyl, wherein said alkyl is unsubstituted or substituted with halogen; preferably each of R₇ and R₉ is independently C₁₋₄alkyl; more preferably each of R₇ and R₉ is independently C₁₋₂alkyl.

9. The compound of any one of claims 1-8, wherein R₇ and R₉ are each independently hydrogen, methyl, ethyl, isopropyl, n-propyl provided that at least one of R₇ and R₉ is not hydrogen.

10. The compound of any one of claims 1-9, wherein R₇ is methyl, and R₉ is methyl; or R₇ is ethyl, and R₉ is ethyl; or R₇ is methyl, and R₉ is ethyl.

11. The compound of any one of claims 1-10, wherein Rₓ and R₁₀ are each hydrogen.

12. The compound of any one of claims 8-11, wherein the 2-position carbon on the piperazine ring is S-configuration, and the 5-position carbon on the piperazine ring is R-configuration.

13. The compound of any one of claims 1-12, wherein L₁ is a direct bond, —C(R_{L1})(R_{L2}), wherein said each of R_{L1}, R_{L2}, is independently hydrogen or C₁₋₄alkyl optionally substituted with deuterium, halogen; preferably L, is a direct bond, —CH₂—, —CH(CH₃)—, —CH(CD₃)-, —CH(CH₂CH₃)—, —CH(C₃H₇)—, —CH(CHF₂)—, or —C(CH₃)₂—; more preferably L₁ is —CH(CH₂CH₃)—, —CH(CH₃)—, or CH(CD₃)-.

14. The compound of any one of claims 1-13, wherein Cy₁ is aryl, heterocyclyl, heteroaryl, or cycloalkyl, each of which is unsubstituted or substituted with one, two or three substituents Ra, wherein each R₃ₐ is independently selected from hydroxy, alkoxy, alkyl, halogen, aminoalkyl, cycloalkyl, R_{3b}—C(O)—N(R_{3c})—, N(R_{3b}R_{3c})—C(O)—, cycloalkyl, heterocyclyl or heterocyclyloxy, each alkyl moiety of which is unsubstituted or substituted with halogen, alkoxy, hydroxy or heterocyclyl; wherein said cycloalkyl or heterocyclyl is unsubstituted or substituted with alkoxy, alkyl, halogen, or hydroxy, wherein R_{3b} and R_{3c} are each independently hydrogen or alkyl, or R_{3b} and R_{3c} together form a bridge containing at least one —CH₂— moiety; preferably Ria is selected from fluoro, bromo, chloro, methyl, ethyl, isopropyl, trifluoromethyl, hydroxyl, 1-methylpyrrolyl, 1-methylazetidinyl, 1-methylpiperidinyl, dimethylaminomethyl, 1,1-difluoroethyl, 3,3-difluoro-1-methylazetidinyl, dimethylaminyl, 1,1-dimethylmethyl, methoxy, cyclopropyl, cyclobutane, 2-fluoro-2-methylethyl, oxetan-3-ylmethyloxy, oxolane-3-ylmethyloxy, oxane-4-ylmethyloxy, oxetan-3-yloxy, oxolane-3-yloxy, oxane-3-yloxy, oxetan-3-methyl-3-yloxy, difluoromethoxy, 2-methoxyethoxy, (2-methoxyethoxy)methyl, 1,1-dioxidethiomorpholine-4-methyl, isopropoxy, dimethylcarbamoyl, (3-hydroxyazetidin-1-yl) methanone, formylamino, acetamido, propionamino, cyclopropoxy or amino.

15. The compound of any one of claims 1-14, wherein Cy₁ is phenyl.

16. The compound of claim 15 wherein Cy₁ is phenyl, which is substituted with one R₃ₐ as disclosed herein at position 4 and optionally substituted with R₃ₐ on the other positions.

17. The compound of any one of claims 1-16, wherein Cy₁ is a monocyclic 5- to 9-membered heterocyclyl or a bicyclic 7- to 10-membered heterocyclyl which is unsubstituted or substituted with one, two or three R₃ₐ.

18. The compound of claim 17, wherein monocyclic 5- to 9-membered heterocyclyl is 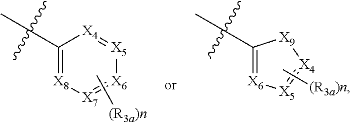
which is unsubstituted or substituted with one, two or three R₃ₐ, wherein each of X₄, X₅, X₆, X₇ and X₈ is independently selected from N or C, X₉ is C, N, S or O, provided that X₄, X₅, X₆, X₇ and X₈ are each independently unsubstituted when X₄, X₅, X₆, X₇ and X₈ are N.

19. The compound of claim 17, wherein said monocyclic 5- to 9-membered heterocyclyl is thiazole, isothiazole, triazole, pyridine, pyrazine, pyrimidine, each of which is unsubstituted or substituted with one, two or three R₃ₐ.

20. The compound of claim 17, wherein said monocyclic 5- to 9-membered heteroaryl is pyridin-2-yl, pyridin-3-yl, pyridin-4-yl, thiazole-2-yl, thiazole-4-yl, isothiazole-3-yl, isothiazole-4-yl, pyrazine-1-yl, pyrazine-2-yl, pyrimidin-2-yl, pyrimidin-4-yl, pyrimidin-5-yl, each of which is unsubstituted or substituted with one, two or three R₃ₐ.

21. The compound of claim 17, wherein said monocyclic 5- to 9-membered heteroaryl ((4,4-difluoropiperidin-1-yl)methyl)pyridin-2-yl, 4-(oxetan-3-ylmethyl)phenyl, 6-aminopyridin-3-yl, 4-((4-methoxypiperidin-1-yl)methyl)phenyl, 4-(cyanomethyl)-phenyl, 4-cyano-phenyl, 4-((1,1-dioxidothiomorpholino)methyl)phenyl, 4-(methylcarbamoyl)phenyl, (4-methoxypiperidine-1-carbonyl)phenyl, 4-((3-methoxypropyl)methyl)carbamoyl)phenyl, 4-((methylsulfonyl)methyl)phenyl, 6-(1,1-difluoroethyl)pyridin-3-yl, 5-cyclopropoxypyridin-2-yl, 4-(3-methoxypyrrolidine-1-carbonyl)phenyl, 4-(methoxymethyl)phenyl, The compound of claim 17, wherein said bicyclic 7- to 10-membered heteroaryl is 6,7-dihydro-5H-cyclopenta[b]pyridine, 6,7-dihydro-5H-cyclopenta[c]pyridine, 2,3-dihydro-1H-indene, 2,3-dihydrobenzofurane, benzo[d][1,3]dioxole, 2,3-dihydrobenzo[b][1,4]dioxine, 4,5,6,7-tetrahydrobenzo[d]thiazole, 4,5,6,7-tetrahydrobenzo[d]isothiazole, quinoxalin, benzo[d]oxazole, benzo[d]thiazole, thiazolo[5,4-b]pyridine, thiazolo[4,5-b]pyridine, benzo[b]thiophene, benzofurane, benzo[c][1,2,5]thiadiazole, 1H-imidazo[4,5-b]pyridine, [1,2,4]triazolo[1,5-a]pyridine, pyrazolo[1,5-a]pyridine, imidazo[1,2-a]pyridine, pyrazolo[1,5-a]pyridine, imidazo[1,5-a]pyridine, benzo[c]isothiazole, thiazolo[4,5-c]pyridine, thiazolo[4,5-b]pyridine thieno[2,3-b]pyridin, imidazo [1,2-b]pyridazine, pyrazolo[1,5-a]pyrimidine, thieno[3,2-b]pyridine, pyrazolo[1,5-a]pyridine, [1,2,4]triazolo[4,3-a]pyridine, phthalazine, isoquinoline, 2,3-dihydrofuro[2,3-c]pyridine, 2,3-dihydrofuro[2,3-b]pyridine, each of which is unsubstituted or substituted with one, two or three R₃ₐ.

22. The compound of claim 17, wherein said bicyclic 7- to 10-membered heteroaryl is 6,7-dihydro-5H-cyclopenta[b]pyridine-2-yl, 6,7-dihydro-5H-cyclopenta[b]pyridine-2-yl, 6,7-dihydro-5H-cyclopenta[c]pyridine-3-yl, 6,7-dihydro-5H-cyclopenta[c]pyridine-3-yl, 2,3-dihydro-1H-indene-5-yl, 2,3-dihydrobenzofuran-5-yl, 2,3-dihydrobenzofuran-6-yl, benzo[d][1,3]dioxole-5-yl, 2,3-dihydrobenzo[b][1,4]dioxine-6-yl, 4,5,6,7-tetrahydrobenzo[d]thiazole-2-yl, 4,5,6,7-tetrahydrobenzo[d]isothiazole-3-yl, quinolin-2-yl, quinolin-6-yl, benzo[d]oxazole-5-yl, benzo[d]oxazole-6-yl, benzo[d]thiazole-5-yl, benzo[d]thiazole-6-yl, thiazolo[5,4-b]pyridine-5-yl, thiazolo[5,4-b]pyridine-6-yl, thiazolo[4,5-b]pyridine-5-yl, thiazolo[4,5-b]pyridine-6-yl, benzo[b]thiophene-6-yl, benzofuran-6-yl, benzo[c][1,2,5]thiadiazole-5-yl, 1H-imidazo[4,5-b]pyridine-5-yl, 1H-imidazo[4,5-b]pyridine-6-yl, [1,2,4]triazolo[1,5-a]pyridine-5-yl, [1,2,4]triazolo[1,5-a]pyridine-6-yl, pyrazolo[1,5-a]pyridine, 5-yl, pyrazolo[1,5-a]pyridine-6-yl, imidazo[1,2-a]pyridine-5-yl, imidazo[1,2-a]pyridine-6-yl, pyrazolo[1,5-a]pyridine-5-yl, imidazo[1,5-a]pyridine-5-yl, imidazo[1,5-a]pyridine-6-yl, benzo[c]isothiazole-5-yl, benzo[c]isothiazole-6-yl, thiazolo[4,5-c]pyridine-5-yl, thiazolo[4,5-c]pyridine-6-yl, thiazolo[4,5-b]pyridine-5-yl, thiazolo[4,5-b]pyridine-6-yl, thieno[2,3-b]pyridin-6-yl, thieno[2,3-b]pyridin-5-yl, thiazolo[5,4-b]phthalaz-2-yl, phthalazi-6-yl, phthalazi-5-yl, imidazo [1,2-b]pyridazin-6-yl, imidazo [1,2-b]pyridazine-5-yl, imidazo[1,2-b]pyridazine-2-yl pyrazolo[1,5-a]pyrimidin-5-yl, pyrazolo[1,5-a]pyrimidin-6-yl, thieno[3,2-b]pyridine-5-yl, thieno[3,2-b]pyridine-6-yl, pyrazolo[1,5-a]phthalaz-5-yl, pyrazolo[1,5-a]phthalaz-6-yl, [1,2,4]triazolo[4,3-a]phthalaz-7-yl, [1,2,4]triazolo[4,3-a]phthalaz-6-yl, phthalazin-6-yl, phthalazin-5-yl, isoquinolin-6-yl, isoquinolin-5-yl, 2,3-dihydrofuro[2,3-c]pyridin-5-yl, 2,3-dihydrofuro[2,3-c]pyridin-6-yl, 2,3-dihydrofuro[2,3-b]pyridine-6-yl, or 2,3-dihydrofuro[2,3-b]pyridine-5-yl, each of which is unsubstituted or substituted with one, two or three R₃ₐ.

23. The compound of any one of claims 1-14, wherein Cy¹ is 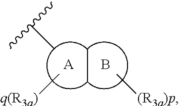
wherein each of ring A and ring B is, independently, a fused aryl, fused 5- to 6-membered heteroaryl, or fused 5- to 6-membered heterocyclyl;
and each of p and q is, independently, 0, 1, 2, 3, provided the sum of p and q is less than or equal to 3.

24. The compound of claim 23, wherein ring A is fused 6-membered heterocyclyl, fused 6-membered aryl or fused 6-membered cycloalkyl; preferably pyridinyl, pyrazinyl, pyrimidinyl, pyridazinyl, or phenyl; more preferably pyridinyl pyrimidinyl, or pyridazinyl.

25. The compound of claim 23, wherein ring B is fused 5-membered heterocyclyl, or fused 5-membered heteroaryl; preferably imidazol, thiazolyl, [1,2,4]triazolyl, tetrahydroimidazolyl, thiazolyl, pyrazolyl, thiazolyl, thienyl, oxazolyl, thiadiazolyl, thienyl, 1,2-dihydrothiazolyl, oxadiazolyl, [1,2,3]triazolyl, dioxolyl, or furanyl; more preferably thiazolyl, pyrazolyl, thiophenyl, or imidazol.

26. The compound of any one of claims 23-25, wherein

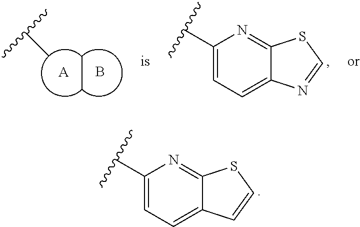

27. The compound of any one of claims 23-25, wherein is

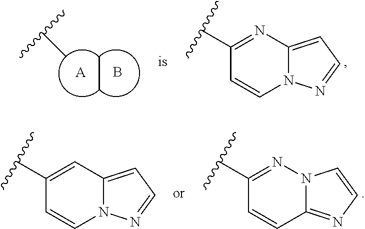

28. The compound of any one of claims 1-14, wherein Cy₁ is 4-(3-methoxyazetidine-1-carbonyl)phenyl, N,N-dimethylbenzamide-4-yl, 2,6-difluoro-N,N-dimethylbenzamide-4-yl, 2-fluoro-N,N-dimethylbenzamide-4-yl, 4-((1,1-dioxidothiomorpholino)methyl)-2,6-difluorophenyl, 2,4-dimethylphenyl, 4-ethylphenyl, 3-methyl-4-(trifluoromethyl)phenyl, 4-(1,1-difluoroethyl)phenyl, 4-(2-methoxyethoxy)phenyl, 2-fluoro-4-methoxyphenyl, 2-fluoro-4-((tetrahydrofuran-3-yl)oxy)phenyl, 4-methoxy-2-methylphenyl, 4-(((tetrahydro-2H-pyran-4-yl)oxy)methyl)phenyl, 4-((oxetan-3-yloxy)methyl)phenyl, 4-(((tetrahydrofuran-3-yl)oxy)methyl)phenyl, 4-((2-methoxyethoxy)methyl)phenyl, 4-(difluoromethoxy)phenyl, 3-fluoro-4-methoxyphenyl, 3-methyl-4-(oxetan-3-yloxy)phenyl, 2-fluoro-4-(oxetan-3-yloxy)phenyl, 3-fluoro-4-(oxetan-3-yloxy)phenyl, 4-((3-methyloxetan-3-yl)oxy)phenyl, 4-((dimethylamino)methyl)-3,5-difluorophenyl, 4-(pyrrolidin-1-ylmethyl)phenyl, 4-((3,3-difluoroazetidin-1-yl)methyl)phenyl, 4-((dimethylamino)methyl)phenyl, 4-((methylamino)methyl)phenyl, 4-(1-methyl-1H-1,2,3-triazol-4-yl)phenyl, 4-isopropylphenyl, 4-cyclopropoxyphenyl, 4-isopropoxyphenyl, 4-methoxyphenyl, 4,5-dimethylthiazol-2-yl, 4,5,6,7-tetrahydrobenzo[d]thiazol-2-yl, 6-cyclopropoxypyridin-3-yl, 6-cyclopropylpyridin-3-yl, 6-cyclopropyl-5-fluoropyridin-3-yl, 6-methoxypyridin-3-yl, 6-(methoxymethyl)pyridin-3-yl, 6-isopropylpyridin-3-yl, 6-ethylpyridin-3-yl, 6-cyclopropyl-2-fluoropyridin-3-yl, 6-(2-fluoropropan-2-yl)pyridin-3-yl, 6-cyclopropyl-4-fluoropyridin-3-yl, 2-cyclopropylpyrimidin-5-yl, 6-methyl-5-(trifluoromethyl)pyridin-2-yl, 5-isopropoxypyridin-2-yl, 5-cyclopropylpyridin-2-yl, 3,5-dimethylpyridin-2-yl, 5,6-dimethylpyridin-2-yl, 5-methoxy-6-methylpyridin-2-yl, 6-(methoxymethyl)-5-methylpyridin-2-yl, 5-cyclopropyl-3-methylpyridin-2-yl, 5-cyclopropyl-6-methylpyridin-2-yl, 5-methoxypyridin-2-yl, 5-methoxy-3-methylpyridin-2-yl, 1-(5-(methoxymethyl)pyridin-2-yl, 5-cyclopropyl-6-fluoropyridin-2-yl, 5-cyclopropyl-4-fluoropyridin-2-yl, 7-fluoro-2,2-dimethyl-2,3-dihydrobenzo[b][1,4]dioxin-6-yl, 2,3-dihydro-1H-inden-5-yl, 1,1-dimethyl-2,3-dihydro-1H-inden-5-yl, 2,3-dihydrobenzofuran-5-yl, benzo[d][1,3]dioxol-5-yl, 4-fluoro-2,2-dimethylbenzo[d][1,3]dioxol-5-yl, 2,2-dimethylbenzo[d][1,3]dioxol-5-yl, 6,7-dihydro-5H-cyclopenta[b]pyridin-2-yl, 2,3-dihydrofuro[3,2-b]pyridin-5-yl, 2,3-dihydrofuro[2,3-b]pyridin-5-yl, [1,3]dioxolo[4,5-b]pyridin-5-yl, 3-fluoropyrazolo[1,5-a]pyridin-6-yl, pyrazolo[1,5-a]pyridin-6-yl, 3-methyl-3H-imidazo[4,5-b]pyridin-6-yl, 3-methylimidazo[1,2-a]pyridin-7-yl, imidazo[1,2-a]pyridin-7-yl, [1,2,4]triazolo[1,5-a]pyridin-6-yl, imidazo[1,2-a]pyridin-6-yl, imidazo[1,5-a]pyridin-6-yl, benzo[d]oxazol-6-yl, 7-fluoro-2-methylbenzo[d]oxazol-6-yl, 4-acetamido-2-fluoro-3-hydroxyphenyl, benzo[c]isothiazol-5-yl, benzo[b]thiophen-6-yl, benzo[d]thiazol-6-yl, 2-methylbenzo[d]thiazol-6-yl, 2-ethylbenzo[d]thiazol-6-yl, thiazolo[5,4-b]pyridin-5-yl, thiazolo[4,5-b]pyridin-6-yl, 4-fluorobenzo[d]thiazol-6-yl, 7-fluorobenzo[d]thiazol-6-yl, 5-fluorobenzo[d]thiazol-6-yl, 7-fluoro-2-methylbenzo[d]thiazol-6-yl, benzo[d]oxazol-5-yl, benzo[c][1,2,5]thiadiazol-5-yl, benzo[d]thiazol-5-yl, 2-methylbenzo[d]thiazol-5-yl, 2-ethylbenzo[d]thiazol-5-yl, 4-fluorobenzo[d]thiazol-5-yl, 3-methylquinoxalin-2-yl, 5-fluoro-2-methylbenzo[d]thiazol-6-yl, 2-methylthiazolo[5,4-b]pyridin-5-yl, thieno[2,3-b]pyridin-6-yl, 2-methylthieno[2,3-b]pyridin-6-yl, 2-fluorothieno[2,3-b]pyridin-6-yl, 6-(1,1-difluoroethyl)pyridin-3-yl, 4-trifluoromethyl-phenyl, 4-fluoro-2-methylbenzo[d]thiazol-5-yl, 2-(methoxymethyl)thiazolo[5,4-b]pyridin-5-yl, 2-(difluoromethyl)thieno[2,3-b]pyridine-6-yl, 2-(methoxymethyl)benzo[d]thiazol-6-yl, 2-cyclopropoxythiazolo[5,4-b]pyridin-5-yl, 2-(2-fluoroethoxy)thiazolo[5,4-b]pyridin-5-yl, 2-(difluoromethyl)thieno[2,3-b]pyridine-6-yl, 2-methylimidazo [1,2-b]pyridazin-6-yl, 3-methylpyrazolo[1,5-a]pyrimidin-5-yl, 2-methylpyrazolo[1,5-a]pyrimidin-5-yl, 2-ethylimidazo [1,2-b]pyridazin-6-yl, pyrazolo[1,5-a]pyrimidin-5-yl, 2-methylthieno[3,2-b]pyridin-5-yl, 2,3-dimethylpyrazolo[1,5-a]pyrimidin-5-yl, 2-fluoropyrazolo[1,5-a]pyrimidin-5-yl, pyrazolo[1,5-a]pyridin-5-yl, 3-fluoro-2-methylpyrazolo[1,5-a]pyrimidin-5-yl, 3-fluoropyrazolo[1,5-a]pyrimidin-5-yl, 2-methylpyrazolo[1,5-a]pyridin-5-yl, pyrazolo[1,5-a]pyrimidin-5-yl, 3-fluoropyrazolo[1,5-a]pyridin-5-yl, 3-chloroimidazo[1,2-b]pyridazin -6-yl, 3-fluoroimidazo[1,2-b]pyridazin-6-yl, 3-fluoro-2-methylpyrazolo[1,5-a]pyridin-5-yl, 2-chloropyrazolo[1,5-a]pyrimidin-5-yl, 2-chloro-3-fluoropyrazolo[1,5-a]pyrimidin-5-yl, 7-fluoro-3,3-dimethyl-2,3-dihydrobenzo[b][1,4]dioxin-6-yl, imidazo[1,2-b]pyridazin-2-yl, thiazolo[5,4-b]pyridin-2-yl, [1,2,4]triazolo[4,3-a]pyridin-7-yl, phthalazin-6-yl, phthalazin-5-yl, 1-methylisoquinolin-6-yl, 2,3-dihydrofuro[2,3-c]pyridin-5-yl, 2,2-difluorobenzo[d][1,3]dioxol-5-yl, 2,3-dihydrofuro[2,3-b]pyridin-6-yl, 4-((3-methoxypyrrolidin-1-yl)methyl)phenyl, 3-methylpyrazolo[1,5-a]pyridine-6-yl, quinoxalin-2-yl.

29. A compound is 2-(7-((2S,5R)-2,5-diethyl-4-(1-(4-(3-methoxyazetidine-1-carbonyl)phenyl)ethyl)piperazin-1-yl)-4-methyl-5-oxo-4,5-dihydro-2H-pyrazolo[4,3-b]pyridin-2-yl)acetonitrile, 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-N,N-dimethylbenzamide, 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-2,6-difluoro-N,N-dimethylbenzamide, 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-2-fluoro-N,N-dimethylbenzamide, 2-(7-((2S,5R)-4-(1-(4-((1,1-dioxidothiomorpholino)methyl)-3,5-difluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,4-dimethylphenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-ethylphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methyl-4-(trifluoromethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-methoxy-3-methylphenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-(1,1-difluoroethyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(2-methoxyethoxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-fluoro-4-methoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-fluoro-4-((tetrahydrofuran-3-yl)oxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-methoxy-2-methylphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(((tetrahydro-2H-pyran-4-yl)oxy)methyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-((oxetan-3-yloxy)methyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(((tetrahydrofuran-3-yl)oxy)methyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-((2-methoxyethoxy)methyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-(difluoromethoxy)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoro-4-methoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methyl-4-(oxetan-3-yloxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-fluoro-4-(oxetan-3-yloxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoro-4-(oxetan-3-yloxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-((3-methyloxetan-3-yl)oxy)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-((dimethylamino)methyl)-3,5-difluorophenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(pyrrolidin-1-ylmethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-((3,3-difluoroazetidin-1-yl)methyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-((dimethylamino)methyl)phenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-((methylamino)methyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(1-methyl-1H-1,2,3-triazol-4-yl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-isopropylphenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4-cyclopropoxyphenyl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-isopropoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-methoxyphenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(4,5-dimethylthiazol-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4,5,6,7-tetrahydrobenzo[d]thiazol-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-cyclopropoxypyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(6-cyclopropylpyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-4-(1-(6-cyclopropylpyridin-3-yl)ethyl)-2,5-diethylpiperazin-1-yl)-4-methyl-2,4-dihydro-5H-pyrazolo[4,3-b]pyridin-5-one, 2-(7-((2S,5R)-4-(1-(6-cyclopropyl-5-fluoropyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-methoxypyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-(methoxymethyl)pyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-isopropylpyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-ethylpyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-cyclopropyl-2-fluoropyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-(2-fluoropropan-2-yl)pyridin-3-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-cyclopropyl-4-fluoropyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(6-cyclopropylpyridin-3-yl)ethyl)-2,5-diethylpiperazin-1-yl)-6-fluoro-4-methyl-5-oxo-4,5-dihydro-2H-pyrazolo[4,3-b]pyridin-2-yl)acetonitrile, 2-(7-((2S,5R)-4-(1-(2-cyclopropylpyrimidin-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-methyl-5-(trifluoromethyl)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-isopropoxypyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5-cyclopropylpyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(3,5-dimethylpyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(5,6-dimethylpyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-methoxy-6-methylpyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(6-(methoxymethyl)-5-methylpyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5-cyclopropyl-3-methylpyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(5-cyclopropyl-6-methylpyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-methoxypyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-methoxy-3-methylpyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-(methoxymethyl)pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5-cyclopropyl-6-fluoropyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(5-cyclopropyl-4-fluoropyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(7-fluoro-2,2-dimethyl-2,3-dihydrobenzo[b][1,4]dioxin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,3-dihydro-1H-inden-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(1,1-dimethyl-2,3-dihydro-1H-inden-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,3-dihydrobenzofuran-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(benzo[d][1,3]dioxol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(4-fluoro-2,2-dimethylbenzo[d][1,3]dioxol-5-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,2-dimethylbenzo[d][1,3]dioxol-5-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluoro-2,2-dimethylbenzo[d][1,3]dioxol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6,7-dihydro-5H-cyclopenta[b]pyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,3-dihydrofuro[3,2-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,3-dihydrofuro[2,3-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-([1,3]dioxolo[4,5-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoropyrazolo[1,5-a]pyridin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyrazolo[1,5-a]pyridin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methyl-3H-imidazo[4,5-b]pyridin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methylimidazo[1,2-a]pyridin-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(imidazo[1,2-a]pyridin-7-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-([1,2,4]triazolo[1,5-a]pyridin-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(imidazo[1,2-a]pyridin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(imidazo[1,5-a]pyridin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(benzo[d]oxazol-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(benzo[d]oxazol-6-yl)ethyl)-5-ethyl-2-methylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(7-fluoro-2-methylbenzo[d]oxazol-6-yl)ethyl)piperazin-1-yl)-4-methyl-5-oxo-4,5-dihydro-2H-pyrazolo[4,3-b]pyridin-2-yl)acetonitrile, N-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-3-fluoro-2-hydroxyphenyl)acetamide, 2-(7-((2S,5R)-4-(1-(benzo[c]isothiazol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(benzo[b]thiophen-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(benzo[d]thiazol-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methylbenzo[d]thiazol-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(benzo[d]thiazol-6-yl)ethyl)-5-ethyl-2-methylpiperazin-1-yl)-4-methyl-5-oxo-4,5-dihydro-2H-pyrazolo[4,3-b]pyridin-2-yl)acetonitrile, 2-(7-((2S,5R)-4-(1-(benzo[d]thiazol-6-yl)ethyl)-2,5-dimethylpiperazin-1-yl)-4-methyl-5-oxo-4,5-dihydro-2H-pyrazolo[4,3-b]pyridin-2-yl)acetonitrile, 7-((2S,5R)-4-(1-(benzo[d]thiazol-6-yl)ethyl)-2,5-diethylpiperazin-1-yl)-2-(cyanomethyl)-4-methyl-5-oxo-4,5-dihydro-2H-pyrazolo[4,3-b]pyridine-6-carbonitrile, 2-(7-((2S,5R)-4-(1-(benzo[d]thiazol-6-yl)ethyl)-2,5-diethylpiperazin-1-yl)-6-bromo-4-methyl-5-oxo-4,5-dihydro-2H-pyrazolo[4,3-b]pyridin-2-yl)acetonitrile, 2-(7-((2S,5R)-2,5-diethyl-4-(1-(2-ethylbenzo[d]thiazol-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(thiazolo[4,5-c]pyridin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(thiazolo[4,5-b]pyridin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(thiazolo[5,4-b]pyridin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(thiazolo[5,4-b]pyridin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluorobenzo[d]thiazol-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(7-fluorobenzo[d]thiazol-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(5-fluorobenzo[d]thiazol-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-4-(1-(7-fluoro-2-methylbenzo[d]thiazol-6-yl)ethyl)-2-methylpiperazin-1-yl)-4-methyl-5-oxo-4,5-dihydro-2H-pyrazolo[4,3-b]pyridin-2-yl)acetonitrile, 2-(7-((2S,5R)-4-(1-(benzo[d]oxazol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(benzo[c][1,2,5]thiadiazol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(benzo[d]thiazol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methylbenzo[d]thiazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-ethylbenzo[d]thiazol-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-fluorobenzo[d]thiazol-5-yl)ethyl)piperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-5-ethyl-2-methyl-4-(1-(3-methylquinoxalin-2-yl)ethyl)piperazin-1-yl)-4-methyl-2,4-dihydro-5H-pyrazolo[4,3-b]pyridin-5-one, 7-((2S,5R)-4-(1-(benzo[d]thiazol-6-yl)ethyl)-5-ethyl-2-methylpiperazin-1-yl)-2-(but-2-yn-1-yl)-4-methyl-2,4-dihydro-5H-pyrazolo[4,3-b]pyridin-5-one, 7-((2S,5R)-4-(1-(benzo[d]thiazol-6-yl)ethyl)-2,5-diethylpiperazin-1-yl)-2-(but-2-yn-1-yl)-4-methyl-2,4-dihydro-5H-pyrazolo[4,3-b]pyridin-5-one, 2-(but-2-yn-1-yl)-7-((2S,5R)-2,5-diethyl-4-(1-(2-methylbenzo[d]thiazol-6-yl)ethyl)piperazin-1-yl)-4-methyl-2,4-dihydro-5H-pyrazolo[4,3-b]pyridin-5-one, 2-(but-2-yn-1-yl)-7-((2S,5R)-5-ethyl-2-methyl-4-(1-(2-methylbenzo[d]thiazol-6-yl)ethyl)piperazin-1-yl)-4-methyl-2,4-dihydro-5H-pyrazolo[4,3-b]pyridin-5-one, 2-(but-2-yn-1-yl)-7-((2S,5R)-5-ethyl-4-(1-(7-fluoro-2-methylbenzo[d]thiazol-6-yl)ethyl)-2-methylpiperazin-1-yl)-4-methyl-2,4-dihydro-5H-pyrazolo[4,3-b]pyridin-5-one, 2-(but-2-yn-1-yl)-7-((2S,5R)-2,5-diethyl-4-(1-(thiazolo[5,4-b]pyridin-5-yl)ethyl)piperazin-1-yl)-4-methyl-2,4-dihydro-5H-pyrazolo[4,3-b]pyridin-5-one, 2-(but-2-yn-1-yl)-7-((2S,5R)-5-ethyl-2-methyl-4-(1-(thiazolo[5,4-b]pyridin-5-yl)ethyl)piperazin-1-yl)-4-methyl-2,4-dihydro-5H-pyrazolo[4,3-b]pyridin-5-one, 7-((2S,5R)-4-(1-(benzo[d]thiazol-5-yl)ethyl)-5-ethyl-2-methylpiperazin-1-yl)-2-(but-2-yn-1-yl)-4-methyl-2,4-dihydro-5H-pyrazolo[4,3-b]pyridin-5-one, 7-((2S,5R)-4-(1-(benzo[d]thiazol-5-yl)ethyl)-2,5-diethylpiperazin-1-yl)-2-(but-2-yn-1-yl)-4-methyl-2,4-dihydro-5H-pyrazolo[4,3-b]pyridin-5-one, 7-((2S,5R)-4-(1-(6-cyclopropylpyridin-3-yl)ethyl)-2,5-diethylpiperazin-1-yl)-4-methyl-2-(oxetan-3-yl)-2,4-dihydro-5H-pyrazolo[4,3-b]pyridin-5-one, 7-((2S,5R)-4-(1-(6-cyclopropylpyridin-3-yl)ethyl)-2,5-diethylpiperazin-1-yl)-4-methyl-2-(oxetan-3-ylmethyl)-2,4-dihydro-5H-pyrazolo[4,3-b]pyridin-5-one, 2-(7-((2S,5R)-4-(1-(benzo[d]thiazol-6-yl)ethyl)-2,5-diethylpiperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(2-methylbenzo[d]thiazol-6-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(7-fluoro-2-methylbenzo[d]thiazol-6-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(thiazolo[5,4-b]pyridin-5-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-4-(1-(6-cyclopropylpyridin-3-yl)ethyl)-2,5-diethylpiperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(5-fluoro-2-methylbenzo[d]thiazol-6-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(2-methylthiazolo[5,4-b]pyridin-5-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-5-ethyl-2-methyl-4-(1-(thieno[2,3-b]pyridin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(2-methylthieno[2,3-b]pyridin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-4-(1-(2-fluorothieno[2,3-b]pyridin-6-yl)ethyl)-2-methylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methylthiazolo[5,4-b]pyridin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methylthiazolo[5,4-b]pyridin-5-yl)ethyl)piperazin-1-yl)-4-(methyl-d3)-5-oxo-4,5-dihydro-2H-pyrazolo[4,3-b]pyridin-2-yl)acetonitrile, 2-(7-((2S,5R)-2,5-diethyl-4-(1-(4-(trifluoromethyl) phenyl)propyl)piperazin-1-yl)-4-methyl-5-oxo-4,5-dihydro-2H-pyrazolo[4,3-b]pyridin-2-yl)acetonitrile, 7-((2S,5R)-2,5-diethyl-4-(1-(2-methylthiazolo[5,4-b]pyridin-5-yl)ethyl) piperazin-1-yl)-4-methyl-2-(oxetan-3-yl)-2,4-dihydro-5H-pyrazolo[4,3-b]pyridin-5-one, 2-(but-2-yn-1-yl)-7-((2S,5R)-2,5-diethyl-4-(1-(2-methylthiazolo[5,4-b]pyridin-5-yl)ethyl)piperazin-1-yl)-4-methyl-2,4-dihydro-5H-pyrazolo[4,3-b]pyridin-5-one, 2-(but-2-yn-1-yl)-7-((2S,5R)-2,5-diethyl-4-(1-(7-fluoro-2-methylbenzo[d]thiazol-6-yl)ethyl)piperazin-1-yl)-4-methyl-2,4-dihydro-5H-pyrazolo[4,3-b]pyridin-5-one, 2-(but-2-yn-1-yl)-7-((2S,5R)-2,5-diethyl-4-(1-(4-fluorobenzo[d]thiazol-5-yl)ethyl)piperazin-1-yl)-4-methyl-2,4-dihydro-5H-pyrazolo[4,3-b]pyridin-5-one, or 2-(cyanomethyl)-7-((2S,5R)-2,5-diethyl-4-(1-(2-methylthiazolo[5,4-b]pyridin-5-yl)ethyl)piperazin-1-yl)-4-methyl-5-oxo-4,5-dihydro-2H-pyrazolo[4,3-b]pyridine-6-carbonitrile, 2-(but-2-yn-1-yl)-7-((2S,5R)-2,5-diethyl-4-(1-(4-fluoro-2-methylbenzo[d]thiazol-5-yl)ethyl)piperazin-1-yl)-4-methyl-2,4-dihydro-5H-pyrazolo[4,3-b]pyridin-5-one, 2-(7-((2S,5R)-2,5-diethyl-4-(1-(2-methylthiazolo[5,4-b]pyridin-5-yl)ethyl-2,2,2-d3)piperazin-1-yl)-4-(methyl-d3)-5-oxo-4,5-dihydro-2H-pyrazolo[4,3-b]pyridin-2-yl)acetonitrile, 2-(7-((2S,5R)-2,5-diethyl-4-(1-(2-(methoxymethyl)thiazolo[5,4-b]pyridin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-(difluoromethyl)thieno[2,3-b]pyridin-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-(methoxymethyl)benzo[d]thiazol-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-cyclopropoxythiazolo[5,4-b]pyridin-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-(2-fluoroethoxy) thiazolo[5,4-b]pyridin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2-(difluoromethyl)thieno[2,3-b]pyridin-6-yl)ethyl)-2,5-diethylpiperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(2-methylimidazo [1,2-b]pyridazin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methylimidazo[1,2-b]pyridazin-6-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(3-methylpyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methylpyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-ethylimidazo [1,2-b]pyridazin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-dimethyl-4-(1-(2-methylthieno[3,2-b]pyridin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(2,3-dimethylpyrazolo[1,5-a]pyrimidin-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methylpyrazolo[1,5-a]pyrimidin-5-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(2-fluoropyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(pyrazolo[1,5-a]pyridin-5-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(3-fluoro-2-methylpyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoropyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(2-methylpyrazolo[1,5-a]pyridin-5-yl)ethyl)piperazin-1-yl)-4-methyl-5-oxo-4,5-dihydro-2H-pyrazolo [4,3-b]pyridin-2-yl)acetonitrile, 2-(7-((2S,5R)-2,5-diethyl-4-(1-(pyrazolo[1,5-a]pyrimidin-5-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(3-fluoropyrazolo[1,5-a]pyrimidin-5-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-2,5-diethyl-4-(1-(3-fluoropyrazolo[1,5-a]pyridin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(pyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(3-chloroimidazo[1,2-b]pyridazin-6-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoroimidazo[1,2-b]pyridazin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoro-2-methylpyrazolo[1,5-a]pyridin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-fluoro-2-methylpyrazolo[1,5-a]pyrimidin-5-yl)ethyl)piperazin-1-yl)-4-methyl-5-oxo-4,5-dihydro-2H-pyrazolo[4,3-d]pyrimidin-2-yl)acetonitrile, 2-(7-((2S,5R)-5-ethyl-2-methyl-4-(1-(2-methylpyrazolo[1,5-a]pyrimidin-5-yl)propyl)piperazin-1-yl)-4-methyl-5-oxo-4,5-dihydro-2H-pyrazolo[4,3-b]pyridin-2-yl)acetonitrile, 2-(7-((2S,5R)-5-ethyl-2-methyl-4-(1-(2-methylpyrazolo[1,5-a]pyrimidin-5-yl)ethyl)piperazin-1-yl)-4-methyl-5-oxo-4,5-dihydro-2H-pyrazolo[4,3-b]pyridin-2-yl)acetonitrile, 2-(7-((2S,5R)-5-ethyl-4-(1-(3-fluoro-2-methylpyrazolo[1,5-a]pyrimidin-5-yl)ethyl)-2-methylpiperazin-1-yl)-4-methyl-5-oxo-4,5-dihydro-2H-pyrazolo[4,3-b]pyridin-2-yl)acetonitrile, 2-(7-((2S,5R)-4-(1-(3-chloroimidazo[1,2-b]pyridazin-6-yl)ethyl)-2,5-diethylpiperazin-1-yl)-4-methyl-5-oxo-4,5-dihydro-2H-pyrazolo[4,3-d]pyrimidin-2-yl)acetonitrile, 2-(7-((2S,5R)-4-(1-(2-chloropyrazolo[1,5-a]pyrimidin-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-chloro-3-fluoropyrazolo[1,5-a]pyrimidin-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(2-chloropyrazolo[1,5-a]pyrimidin-5-yl)ethyl)-5-ethyl-2-methylpiperazin-1-yl)-4-methyl-5-oxo-4,5-dihydro-2H-pyrazolo[4,3-b]432yridine-2-yl)acetonitrile, 2-(7-((2S,5R)-4-(1-(2-chloro-3-fluoropyrazolo[1,5-a]pyrimidin-5-yl)ethyl)-5-ethyl-2-methylpiperazin-1-yl)-4-methyl-5-oxo-4,5-dihydro-2H-pyrazolo[4,3-b]pyridin-2-yl)acetonitrile, 2-(7-((2S,5R)-4-(1-(7-fluoro-3,3-dimethyl-2,3-dihydrobenzo[b][1,4]dioxin-6-yl)ethyl)-2,5-dimethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(imidazo[1,2-b]pyridazin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(thiazolo[5,4-b]pyridin-2-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(5-((4,4-difluoropiperidin-1-yl)methyl)pyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(oxetan-3-ylmethyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-([1,2,4]triazolo[4,3-a]pyridin-7-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(benzo[d][1,3]dioxol-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(phthalazin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-aminopyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(1-methylisoquinolin-6-yl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-((4-methoxypiperidin-1-yl)methyl)phenyl)ethyl)piperazin-1-yl)-4-methyl-5-oxo-4,5-dihydro-2H-pyrazolo[4,3-b]pyridin-2-yl)acetonitrile, 2-(4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)phenyl)acetonitrile, 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)benzonitrile, 2-(7-((2S,5R)-4-(1-(4-((1,1-dioxidothiomorpholino)methyl)phenyl)ethyl)-2,5-diethylpiperazin-1-yl)-4-methyl-5-oxo-4,5-dihydro-2H-pyrazolo[4,3-b]pyridin-2-yl)acetonitrile, 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-N-methylbenzamide, 2-(7-((2S,5R)-2,5-diethyl-4-(1-(4-(4-methoxypiperidine-1-carbonyl)phenyl)ethyl)piperazin-1-yl)-4-methyl-5-oxo-4,5-dihydro-2H-pyrazolo[4,3-b]433yridine-2-yl)acetonitrile, 4-(1-((2R,5S)-4-(2-(cyanomethyl)-4-methyl-5-oxo-4,5-dihydro-2H-pyrazolo[4,3-b]pyridin-7-yl)-2,5-diethylpiperazin-1-yl)ethyl)-N-(2-methoxyethyl)-N-methylbenzamide, 2-(7-((2S,5R)-4-(1-(2,3-dihydrofuro[2,3-c]pyridin-5-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-4-(1-(2,2-difluorobenzo[d][1,3]dioxol-5-yl)ethyl)-2,5-diethylpiperazin-1-yl)-4-methyl-5-oxo-4,5-dihydro-2H-pyrazolo[4,3-b]433yridine-2-yl)acetonitrile, 2-(7-((2S,5R)-2,5-diethyl-4-(1-(4-((methylsulfonyl)methyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-4-(1-(6-(1,1-difluoroethyl)pyridin-3-yl)ethyl)-2,5-diethylpiperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-4-(1-(2,3-dihydrofuro[2,3-b]pyridin-6-yl)ethyl)-5-ethyl-2-methylpiperazin-1-yl)-4-methyl-2,4-dihydro-5H-pyrazolo[4,3-b]pyridin-5-one, 2-(7-((2S,5R)-4-(1-(5-cyclopropoxypyridin-2-yl)ethyl)-2,5-diethylpiperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(4-(3-methoxypyrrolidine-1-carbonyl)phenyl)ethyl)piperazin-1-yl)-4-methyl-5-oxo-4,5-dihydro-2H-pyrazolo[4,3-b]pyridine-2-yl)acetonitrile, 2-(7-((2S,5R)-2,5-diethyl-4-(1-(4-((3-methoxypyrrolidin-1-yl)methyl)phenyl)ethyl)piperazin-1-yl)-4-methyl-5-oxo-4,5-dihydro-2H-pyrazolo[4,3-b]pyridin-2-yl)acetonitrile, 2-(7-((2S,5R)-2,5-diethyl-4-(1-(3-methylpyrazolo[1,5-a]pyridine-6-yl)ethyl)piperazin-1-yl)-4-methyl-5-oxo-4,5-dihydro-2H-pyrazolo[4,3-b]pyridine-2-yl)acetonitrile, 2-(7-((2S,5R)-2,5-diethyl-4-(1-(4-(methoxymethyl)phenyl)ethyl)piperazin-1-yl)-4-methyl-5-oxo-4,5-dihydro-2H-pyrazolo[4,3-b]pyridin-2-yl)acetonitrile, 2-(but-2-yn-1-yl)-7-((2S,5R)-2,5-diethyl-4-(1-(quinoxalin-2-yl)ethyl)piperazin-1-yl)-4-methyl-2,4-dihydro-5H-pyrazolo[4,3-b]pyridin-5-one .

30. A compound, or a stereoisomer, or a pharmaceutically acceptable salt thereof, wherein said compounds is any one of the exemplified compound.

31. A pharmaceutical composition comprising one or more compounds of any one of claims 1-30, or a stereoisomer or a pharmaceutically acceptable salt thereof, and a pharmaceutically acceptable excipient.

32. A method of treating cancer, comprising administering to a patient in need of such treatment a therapeutically effective amount of a compound of any one of claims 1-30 or a pharmaceutical composition of claim 30.


SEQUENCE LISTING (2 sequences)

SEQ ID NO: 1 - DNA, 2841 bp, synthetic construct
  source 1..2841: mol_type = other DNA
atgcatcaccatcaccatcaccatcacgagaacctgtacttccagggatccatggaaccccgtgatggtagccccgaagctcgtagctccgattccgagtccgccagcgcttcctcctccggtagcgaacgtgacgctggtcccgagcccgacaaagctccccgtcgtctgaataagcgccgttttcccggtctccgtctgttcggccaccgcaaggccatcactaagtccggtctccagcatctggctcctcctcctcctacccccggtgctccttgctccgaatccgagcgccagattcgctccactgtggattggtccgaaagcgccacctatggtgagcatatctggttcgagaccaacgtctccggcgacttctgttatgtcggtgagcaatactgtgtggctcgtatgctgcagaagtccgtgtcccgccgtaaatgcgccgcttgcaaaatcgtggtccataccccttgcatcgagcaactggagaaaatcaacttccgctgcaagcccagctttcgtgagtccggttcccgcaacgtgcgcgaacctactttcgtgcgccaccactgggtgcatcgtcgtcgccaagacggcaaatgccgccactgcggcaaaggttttcagcagaaattcaccttccacagcaaggagatcgtcgccatcagctgcagctggtgcaaacaagcttaccattccaaagtgagctgcttcatgctccagcagatcgaagagccttgctctctgggtgtgcatgctgctgtcgtgattccccctacttggattctgcgtgctcgccgtccccagaacactctgaaggcctccaaaaagaagaagcgcgccagcttcaagcgtaagagctccaaaaagggtcccgaagagggccgttggcgtcccttcatcatccgccctactccttcccctctgatgaagcctctgctggtcttcgtcaaccctaagagcggcggcaaccaaggtgctaaaatcatccagtccttcctctggtatctgaaccctcgtcaagtgttcgacctcagccaaggcggtcctaaggaggctctggagatgtaccgcaaggtccacaatctgcgcatcctcgcttgtggtggcgatggcaccgtgggctggattctgtccactctggaccaactgcgtctgaaacctcccccccccgtggctattctgcctctcggtaccggcaacgatctggctcgtactctgaattggggtggtggctacaccgatgagcccgtgtccaagattctgtcccacgtcgaagaaggcaatgtcgtccaactggaccgttgggacctccacgccgaacccaaccccgaggctggccccgaggaccgtgacgagggcgctactgaccgtctgcccctcgacgtcttcaataattacttctctctgggctttgacgctcacgtgactctggaatttcatgaaagccgcgaggccaaccccgagaagttcaattcccgtttccgcaacaagatgttctacgctggcaccgccttcagcgacttcctcatgggctccagcaaggacctcgctaagcatatccgcgtggtgtgcgatggcatggatctgacccctaagatccaagatctgaagccccaatgtgtcgtgtttctgaacatcccccgctactgcgctggtactatgccttggggccatcccggtgaacaccatgacttcgaacctcagcgtcatgatgacggctatctggaggtgatcggtttcaccatgacctccctcgctgctctgcaagtgggtggccacggcgaacgtctgactcaatgccgcgaggtggtgctgaccaccagcaaagccatccccgtccaagtggatggtgagccttgcaagctggccgcctcccgtatccgtatcgctctccgcaatcaagctaccatggtccagaaggccaaacgccgcagcgctgctcctctccacagcgaccaacaacccgtccccgaacagctgcgcatccaagtgtcccgtgtcagcatgcatgactacgaggctctgcactacgacaaggaacagctgaaggaagccagcgtgcctctgggtactgtggtcgtgcccggtgacagcgatctggagctctgccgtgcccacatcgagcgtctgcagcaagagcccgacggtgctggtgccaagagccctacttgccaaaaactctcccccaagtggtgtttcctcgacgctaccaccgccagccgcttctaccgcattgatcgcgcccaagagcatctgaactatgtcaccgagatcgctcaagacgagatctacatcctcgaccccgaactcctcggtgctagcgcccgtcccgacctccccactcctacctcccctctgcccacttccccttgttcccccacccctcgtagcctccaaggtgatgctgcccctcctcaaggtgaggagctcattgaggccgctaagcgtaacgatttctgcaagctccaagagctgcatcgtgctggtggcgacctcatgcaccgcgatgagcagagccgcactctgctgcaccacgctgtgtccactggtagcaaggacgtggtgcgctatctgctggaccacgctcctcccgagatcctcgacgctgtggaagaaaacggcgagacttgcctccaccaagctgctgctctgggtcaacgtaccatctgccactacatcgtcgaagctggtgcttctctgatgaagaccgaccagcaaggtgatactccccgtcagcgcgccgagaaagcccaagacaccgaactggctgcctatctggagaaccgtcagcactaccagatgattcagcgtgaagaccaagagaccgccgtgtaa

SEQ ID NO: 2 - protein, 929 aa, synthetic construct
MEPRDGSPEARSSDSESASASSSGSERDAGPEPDKAPRRLNKRRFPGLRLFGHRKAITKSGLQHLAPPPPTPGAPCSESERQIRSTVDWSESATYGEHIWFETNVSGDFCYVGEQYCVARMLQKSVSRRKCAACKIVVHTPCIEQLEKINFRCKPSFRESGSRNVREPTFVRHHWVHRRRQDGKCRHCGKGFQQKFTFHSKEIVAISCSWCKQAYHSKVSCFMLQQIEEPCSLGVHAAVVIPPTWILRARRPQNTLKASKKKKRASFKRKSSKKGPEEGRWRPFIIRPTPSPLMKPLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRWDLHAEPNPEAGPEDRDEGATDRLPLDVFNNYFSLGFDAHVTLEFHESREANPEKFNSRFRNKMFYAGTAFSDFLMGSSKDLAKHIRVVCDGMDLTPKIQDLKPQCVVFLNIPRYCAGTMPWGHPGEHHDFEPQRHDDGYLEVIGFTMTSLAALQVGGHGERLTQCREVVLTTSKAIPVQVDGEPCKLAASRIRIALRNQATMVQKAKRRSAAPLHSDQQPVPEQLRIQVSRVSMHDYEALHYDKEQLKEASVPLGTVVVPGDSDLELCRAHIERLQQEPDGAGAKSPTCQKLSPKWCFLDATTASRFYRIDRAQEHLNYVTEIAQDEIYILDPELLGASARPDLPTPTSPLPTSPCSPTPRSLQGDAAPPQGEELIEAAKRNDFCKLQELHRAGGDLMHRDEQSRTLLHHAVSTGSKDVVRYLLDHAPPEILDAVEENGETCLHQAAALGQRTICHYIVEAGASLMKTDQQGDTPRQRAEKAQDTELAAYLENRQHYQMIQREDQETAV